SEQUENCE LISTING (37 sequences)

SEQ ID NO: 1 - protein, 117 aa, synthetic construct
  REGION 1..117: note = CD33-A VH
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSS

SEQ ID NO: 2 - protein, 111 aa, synthetic construct
  REGION 1..111: note = CD33-A VL
DIVLTQSPDSLAVSLGERATINCRASQSVSTSTYSYMHWYQQKPGQPPKLLIKYASNLESGVPDRFSGSGSGTDFTLTISSLQAEDVAVYYCQHSWEIPLTFGQGTKLEIK

SEQ ID NO: 3 - protein, 117 aa, synthetic construct
  REGION 1..117: note = CD33-B VH
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 4 - protein, 107 aa, synthetic construct
  REGION 1..107: note = CD33-B VL
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 5 - protein, 116 aa, synthetic construct
  REGION 1..116: note = CD33-C VH
EVQLQQSGPEVVKPGASVKISCKASGYTFTDYNMHWVKQAHGQSLEWIGYIYPYNGGTGYNQKFKSKATLTVDNSASTAYMEVRSLTSEDTAVYYCARGRPAMDYWGQGTLVTVSS

SEQ ID NO: 6 - protein, 111 aa, synthetic construct
  REGION 1..111: note = CD33-C VL
EIVLTQSPATLSVSLGQRATISCRASESVDNYGISFMNWFQQKPGQPPKLLIYAASNQGSGVPARFSGSGSGTDFTLTIHPMEEDDTAMYFCQQSKEVPWTFGGGTKLEIK

SEQ ID NO: 7 - protein, 119 aa, synthetic construct
  REGION 1..119: note = PSMA-A VH
QVQLVESGGGLVKPGGSLRLSCAASGFTFSDFYMYWIRQAPGKGLEWVATISDGGGYTSYPDSVKGRFTISRDNAKNSLYLQMNSLRAEDTAVYYCARGLWLRDALDYWGQGTTVTVSS

SEQ ID NO: 8 - protein, 108 aa, synthetic construct
  REGION 1..108: note = PSMA-A VL
EIVLTQSPATLSLSPGERATLSCSASSSISSNYLHWYQQKPGQAPRLLIYRTSNLASGIPARFSGSGSGTDYTLTISRLEPEDFAVYYCQQGSYIPFTFGQGTKLEIK

SEQ ID NO: 9 - protein, 124 aa, synthetic construct
  REGION 1..124: note = PSMA-B VH
QVQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCARVWDYYYDSSGDAFDIWGQGTMVTVSS

SEQ ID NO: 10 - protein, 107 aa, synthetic construct
  REGION 1..107: note = PSMA-B VL
VIWMTQSPSSVSASVGDRVTITCRASQGISSWLAWYQQKPGKAPKLLIYAASNLQSGVPSRFSGSGSGTDFTLTISSLQPEDFATYYCQQANSFPLTFGGGTKVDIK

SEQ ID NO: 11 - protein, 123 aa, synthetic construct
  REGION 1..123: note = PSMA-C VH
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSS

SEQ ID NO: 12 - protein, 107 aa, synthetic construct
  REGION 1..107: note = PSMA-C VH
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIK

SEQ ID NO: 13 - protein, 119 aa, synthetic construct [sequence omitted]

SEQ ID NO: 14 - protein, 107 aa, synthetic construct [sequence omitted]

SEQ ID NO: 15 - protein, 120 aa, synthetic construct [sequence omitted]

SEQ ID NO: 16 - protein, 107 aa, synthetic construct [sequence omitted]

SEQ ID NO: 17 - protein, 120 aa, synthetic construct
  REGION 1..120: note = HER2-C VH
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 18 - protein, 107 aa, synthetic construct
  REGION 1..107: note = HER2-C VL
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 19 - protein, 329 aa, synthetic construct
  REGION 1..329: note = IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 329 aa, synthetic construct
  REGION 1..329: note = IgG1 Heavy Chain Constant Region S239C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 329 aa, synthetic construct
  REGION 1..329: note = IgG1 Heavy Chain Constant Region K334C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 118 aa, synthetic construct
  REGION 1..118: note = CD33 huMy9-6 VH
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 23 - protein, 112 aa, synthetic construct
  REGION 1..112: note = CD33 huMy9-6 VL
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK

SEQ ID NO: 24 - protein, 445 aa, synthetic construct
  REGION 1..445: note = CD33 huMy9-6-IgG4-S228P heavy chain
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 219 aa, synthetic construct
  REGION 1..219: note = CD33 huMy9-6-IgG4-S228P light chain
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 327 aa, synthetic construct
  REGION 1..327: note = IgG4 Heavy Chain Constant Region S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 116 aa, synthetic construct [sequence omitted]

SEQ ID NO: 28 - protein, 111 aa, synthetic construct [sequence omitted]

SEQ ID NO: 29 - protein, 115 aa, synthetic construct
  REGION 1..115: note = PSMA-D VH
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 30 - protein, 107 aa, synthetic construct
  REGION 1..107: note = PSMA-D VL
DIVMTQSHKEMSTSVGDRVSIICKASQDVGTAVDWYQQKPGQSPKLLIYWASTRHTGVPDRFTGSGSGTDFTLAITNVQSEDLADYFCQQYNSYPLTFGAGTKLEIK

SEQ ID NO: 31 - protein, 121 aa, synthetic construct [sequence omitted]

SEQ ID NO: 32 - protein, 106 aa, synthetic construct [sequence omitted]

SEQ ID NO: 33 - protein, 117 aa, synthetic construct [sequence omitted]

SEQ ID NO: 34 - protein, 111 aa, synthetic construct [sequence omitted]

SEQ ID NO: 35 - protein, 451 aa, synthetic construct
  REGION 1..451: note = BCMA-A heavy chain
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTIPPVLDSDGSFFLYSKLTVDKSRWQQGNVFSCSVMHEALHNHYTQKSLSLSPGK

SEQ ID NO: 36 - protein, 214 aa, synthetic construct
  REGION 1..214: note = BCMA-A light chain
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 329 aa, synthetic construct
  REGION 1..329: note = IgG1 N297A Constant regions (CH1-Hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0144230 A1
(43) Pub. Date: May 8, 2025

(54) LINKERS FOR USE IN ANTIBODY DRUG CONJUGATES

(71) Applicant: Orum Therapeutics, Inc., Yuseong-gu (KR)

(72) Inventors: Nathan FISHKIN, Cambridge, MA (US); Chen BAI, Arlington, MA (US); Khuloud TAKROURI, Wayland, MA (US); Peter U. PARK, Cambridge, MA (US)

(21) Appl. No.: 18/690,506

(22) PCT Filed: Sep. 7, 2022
(86) PCT No.: PCT/IB2022/058428

§ 371 Date: Mar. 8, 2024

Related U.S. Application Data
(60) Provisional application No. 63/241,914, filed on Sep. 8, 2021.
(60) Provisional application No. 63/293,591, filed on Dec. 23, 2021.
(60) Provisional application No. 63/351,639, filed on Jun. 13, 2022.
(60) Provisional application No. 63/374,282, filed on Sep. 1, 2022.

(51) Int. Cl. A61K 47/68; A61K 47/54; A61K 47/61; A61P 35/00

(52) U.S. Cl. CPC A61K 47/6851; A61K 47/545; A61K 47/61; A61K 47/6849; A61K 47/6889; A61P 35/00

(57) ABSTRACT

The present disclosure provides traceless linkers, which can link an inducer of protein-protein interaction to a cell binding agent. Also provided are compositions comprising the linked compounds. The compounds and compositions are useful for treating diseases such as cancer in subjects in need thereof.

### CROSS-REFERENCE TO RELATED APPLICATIONS

This application claims the priority benefit of U.S. Provisional Application No. 63/241,914, filed Sep. 8, 2021; U.S. Provisional Application No. 63/293,591, filed Dec. 23, 2021; U.S. Provisional Application No. 63/351,639, filed Jun. 13, 2022; and U.S. Provisional Application No. 63/374,282, filed Sep. 1, 2022, which are incorporated herein by reference in their entireties.

### REFERENCE TO SEQUENCE LISTING SUBMITTED ELECTRONICALLY

The content of the electronically submitted sequence listing (Name 4547_020PC03_Seglisting_ST26; Size: 52,902 bytes; and Date of Creation: Sep. 6, 2022) filed with the application is incorporated herein by reference in its entirety.

### FIELD

The present disclosure provides traceless linkers, which can link an inducer of protein-protein interaction to a cell binding agent. Also provided are compositions comprising the linked compounds. The compounds and compositions are useful for treating diseases in subjects in need thereof.

### BACKGROUND

Protein-protein interactions (PPIs) represent a large class of therapeutic targets both inside and outside the cell. PPIs are central to all biological processes and are often dysregulated in disease. Despite the importance of PPIs in human biology, this target class has been extremely challenging to convert to therapeutics. There is therefore a continuing need for new compounds that can effectively induce PPIs and treat diseases such as cancer.

Targeted protein degraders are currently being studied as a class of molecules that can be used to target and degrade hard to drug proteins. The most well-known targeted protein degraders utilizes proteolysis targeting chimera (PROTAC)-based protein degradation and is an emerging field that holds significant promise for targeting ‘undruggable’ proteins that do not exhibit enzymatic activity and are not amenable to classical inhibition. PROTACs are heterobifunctional small molecules that simultaneously bind a target protein and an E3 ligase, thereby leading to ubiquitination and subsequent degradation of the target. Molecular glues are another type of targeted protein degraders which differ from PROTACs in that they bind to an E3 ligase and alter the shape of the ligase's surface and enable the ligase to bind to the target protein, thereby leading to ubiquitination and degradation of the target. While targeted protein degraders present an exciting opportunity to modulate proteins in a manner independent of enzymatic or signaling activity, preparing compounds that discriminate between cells of different types can be challenging.

Antibody drug conjugates are an innovative therapeutic application that combines the unique high specificity of monoclonal antibodies with the potent activity of small molecule drugs that are often unsuitable for systemic administration. The two agents are tethered through a linker which is typically cleaved at the target site.

The use of traceless linkers in antibody drug conjugates allows for the release of the therapeutic payload with no evidence of linker attachment. Ideally, the linker must possess sufficient stability for the conjugated molecule to localize to its destination without premature cleavage. The linker must also possess the ability to be rapidly cleaved, releasing the payload once internalized into the target cell. Known examples of traceless linkers either require UV light for activation or use a chemical trigger that either has a stability half-life of minutes to hours or gives a mixture of less active byproducts upon activation.

Despite ongoing work, there is still a continuing need to selectively target diseased tissues and cells as well as proteins previously considered ‘undruggable’ with compounds that can induce desirable protein-protein interactions, including those that induce protein degradation.

### SUMMARY

In certain aspects, the present disclosure provides a conjugate of formula (XX):

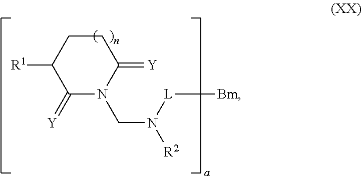

or a pharmaceutically acceptable salt thereof, wherein:
a is from 1 to 10;
n is 0 or 1;
R¹ is a compound that induces a protein-protein interaction;
R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;
each Y is independently S or O;
L is a cleavable linker; and
Bm is a binding moiety that is capable of specifically binding to a protein. In some aspects, the protein is on a cell surface.

In some aspects, the present disclosure provides a conjugate of formula (XXXII):

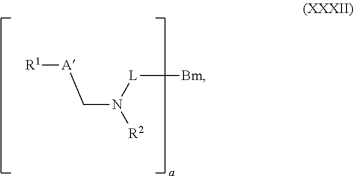

or a pharmaceutically acceptable salt thereof, wherein:
a is from 1 to 10
A′ is

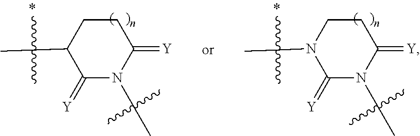

wherein
   n is 0 or 1;
   each Y is independently S or O;
   [symbol] indicates the point of attachment to R¹; and
   [symbol] indicates the point of attachment to the methylene group;
R¹, together with A′, is a compound that induces a protein-protein interaction;
R² is selected from hydrogen, a group that provides stability to R¹-A′, a group that provides solubility to R¹-A′, and a group that provides stability and solubility to R¹-A′;
L is a cleavable linker; and
Bm is a binding moiety that is capable of specifically binding to a protein. In some aspects, the protein is on a cell surface.

In certain aspects, the present disclosure provides a conjugate of formula (XX) or formula (XXXII), or a pharmaceutically acceptable salt thereof, wherein the binding moiety is an antibody, antibody fragment, or an antigen-binding fragment.

In some aspects, the present disclosure provides a conjugate of formula (XX) or formula (XXXII), or a pharmaceutically acceptable salt thereof, wherein a is from 2 to 8.

In some aspects, the present disclosure provides a conjugate of formula (XX) or formula (XXXII), or a pharmaceutically acceptable salt thereof, wherein the linker is cleavable by a protease.

In some aspects, the present disclosure provides a conjugate of formula (XX) or formula (XXXII), or a pharmaceutically acceptable salt thereof, wherein L is selected from the group consisting of

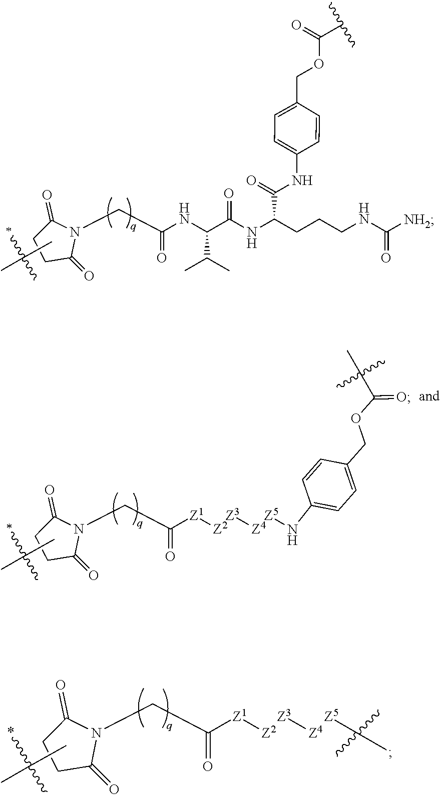

wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues;
[symbol] is the point of attachment to the parent molecular moiety; and
[symbol] is the point of attachment to the binding moiety.

In some aspects, Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

In some aspects:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine;
Z⁴ is selected from the group consisting of L-citrulline, D-citrulline, L-asparagine, D-asparagine, L-lysine, D-lysine, L-phenylalamine, D-phenylalanine, and glycine; and
Z⁵ is absent or glycine.

In some aspects, the present disclosure provides a conjugate of formula (XX) or formula (XXXII), or a pharmaceutically acceptable salt thereof, wherein L is

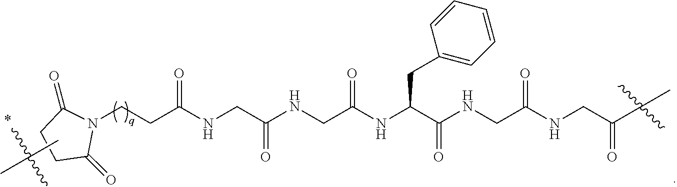

In some aspects, q is 4.

In some aspects, the present disclosure provides a conjugate of formula (XX) or formula (XXXII), or a pharmaceutically acceptable salt thereof, wherein L is a bioreducible linker.

In some aspects, L is selected from the group consisting of

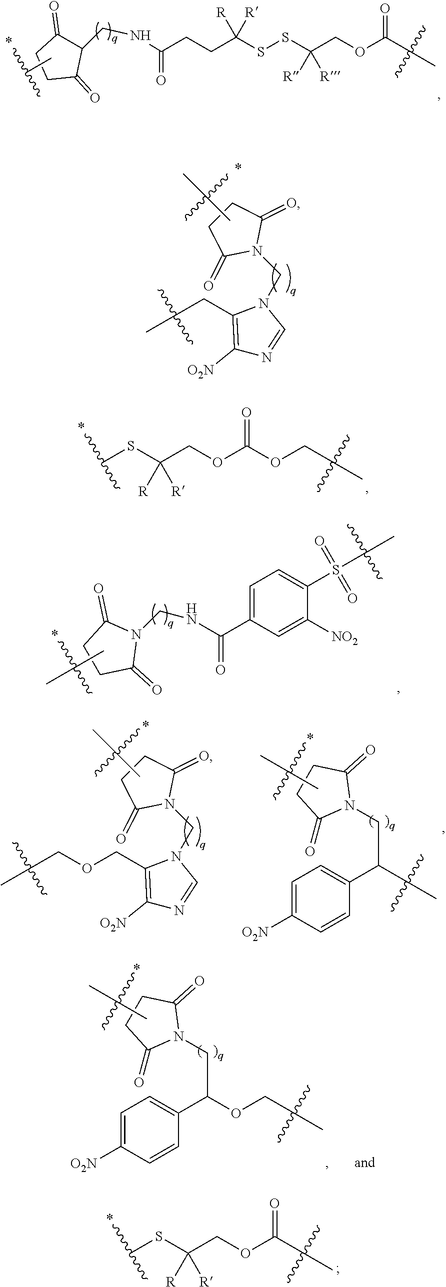

wherein:
   q is from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
   [symbol] is the point of attachment to the parent molecular moiety; and
   [symbol] is the point of attachment to the binding moiety.

In some aspects, L is

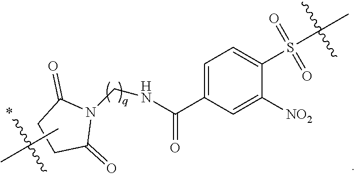

In some aspects, q is 2.

In some aspects, the present disclosure provides a conjugate of formula (XX) or formula (XXXII), or a pharmaceutically acceptable salt thereof, wherein L is a click-to-release linker.

In some aspects, L is

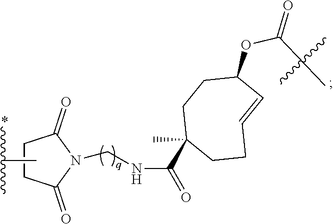

wherein:
   q is from 2 to 10;
   [symbol] is the point of attachment to the parent molecular moiety; and
   [symbol] is the point of attachment to the binding moiety.

In some aspects, the present disclosure provides a conjugate of formula (XX) or formula (XXXII), or a pharmaceutically acceptable salt thereof, wherein L is a beta-glucuronidase cleavable linker.

In some aspects, L is

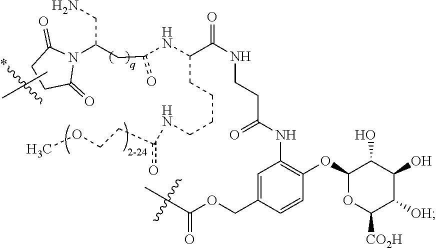

wherein:
   q is from 2 to 10;
   - - - is absent or a bond;
   [symbol] is the point of attachment to the parent molecular moiety; and
   [symbol] is the point of attachment to the binding moiety.

In certain aspects, L is attached to a cysteine, lysine, tyrosine, or glutamine in the Bm. In certain aspects, the cysteine or lysine is an engineered cysteine or lysine. In some aspects, the cysteine or lysine is endogenous to the Bm.

In some aspects, the present disclosure provides a conjugate of formula (XX) or formula (XXXII), or a pharmaceutically acceptable salt thereof, wherein Bm is an antibody or antigen binding portion thereof. In certain aspects, L is attached to an engineered cysteine at heavy chain position S239 and/or K334 of the antibody or antigen binding portion thereof according to EU numbering. In some aspects, L is attached to the glutamine at heavy chain position 295 of the antibody or antigen binding portion thereof according to EU numbering.

In some aspects, the present disclosure provides a conjugate of formula (XX) or formula (XXXII), or a pharmaceutically acceptable salt thereof, wherein the protein that the Bm binds to is a surface antigen, optionally wherein binding of the Bm to the surface antigen results in internalization of the conjugate or pharmaceutically acceptable salt thereof into a cell.

In some aspects, the surface antigen comprises 5T4, ACE, ADRB3, AKAP-4, ALK, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, c13, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-1 1Ra, IL-1 receptor, IL-12 receptor, IL-23 receptor, IL-13 receptor, IL-22 receptor, IL-4 receptor, IL-5 receptor, IL-6 receptor, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-1a, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PD-1, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRP, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some aspects, the surface antigen comprises HER2, CD20, CD38, CD33, BCMA, CD138, EGFR, FGFR4, GD2, PDGFR, or combinations thereof. In some aspects, the surface antigen comprises CD79b. In some aspects, the surface antigen comprises PSMA.

In some aspects, the present disclosure provides a conjugate of formula (XX) or formula (XXXII), or a pharmaceutically acceptable salt thereof, wherein Bm binds to a nuclear hormone receptor. In some aspects, the nuclear hormone receptor is androgen receptor (AR) or estrogen receptor (ER).

In some aspects, the antibody is selected from the group consisting of rituximab, trastuzumab, gemtuzumab, pertuzumab, obinutuzumab, ofatumumab, daratumumab, STI-6129, lintuzumab, huMy9-6, balantamab, indatuximab, cetuximab, dinutuximab, anti-CD38 A2 antibody, huAT15/3 antibody, alemtuzumab, ibritumomab, tositumomab, bevacizumab, panitumumab, tremelimumab, ticilimumab, catumaxomab, oregovomab, and veltuzumab.

In some aspects, the antibody is rituximab, trastuzumab, pertuzumab, huMy9-6, lintuzumab, or gemtuzumab. In some aspects, the antibody is polatuzumab, J591, or belantamab. In some aspects, the antibody is CD33-D.

In some aspects, the present disclosure provides a conjugate of formula (XX) or formula (XXXII) or a pharmaceutically acceptable salt thereof, wherein R² is a group that provides stability to the conjugate. In some aspects, R² is selected from C₂-C₆alkenyl, C₁-C₆alkyl; C₂-C₆alkynyl, benzyl, C₃-C₆cycloalkyl, and C₃-C₆cycloalkyl(C₁-C₃alkyl).

In some aspects, the present disclosure provides a conjugate of formula (XX) or formula (XXXII), or a pharmaceutically acceptable salt thereof, wherein R² is hydrogen or C₁-C₆alkyl. In some aspects, R² is methyl.

In some aspects, the present disclosure provides a conjugate of formula (XX) or formula (XXXII), or a pharmaceutically acceptable salt thereof, wherein R² is methyl.

In some aspects, the present disclosure provides a conjugate of formula (XX) or formula (XXXII), or a pharmaceutically acceptable salt thereof, wherein R² is a group that provides solubility to the conjugate. In some aspects, R² is selected from:

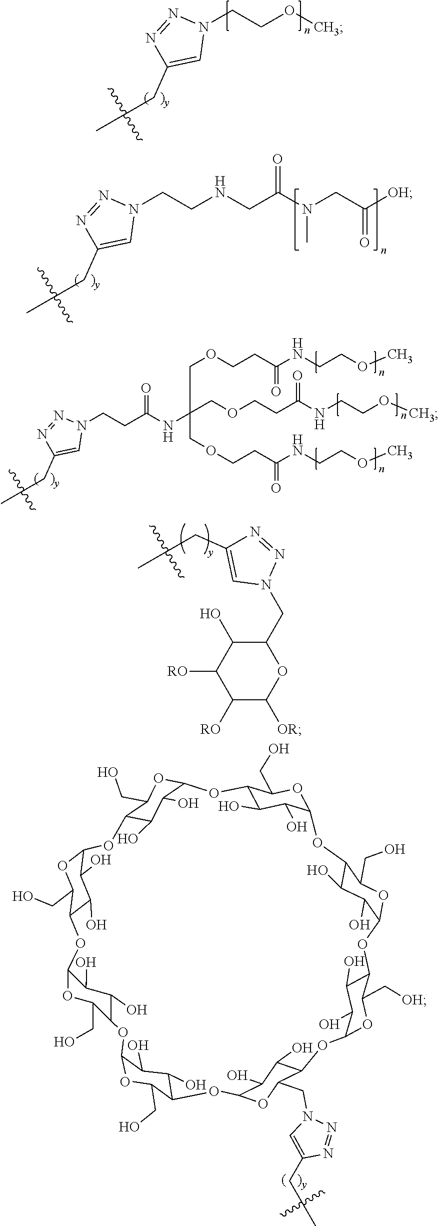

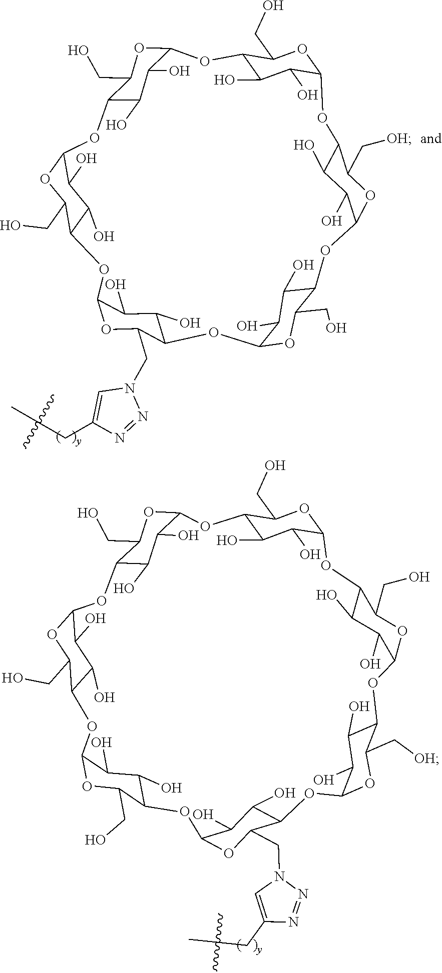

wherein:
each n is independently 1, 2, 3, 4, or 5;
each y is independently 1 or 2; and
each R is independently hydrogen, C₆H₁₁O₅, C₁₂H₂₁O₁₀, C₁₈H₃₁O₁₅, or C₂₄H₄₁O₂₀.

In some aspects, the present disclosure provides a conjugate of formula (XX) or formula (XXXII), or a pharmaceutically acceptable salt thereof, wherein each Y is O.

In some aspects, the present disclosure provides a conjugate of formula (XX), or a pharmaceutically acceptable salt thereof, wherein R¹ is a PPI modulator. In some aspects, the present disclosure provides a conjugate of formula (XXXII), or a pharmaceutically acceptable salt thereof, wherein R¹-A′ is a PPI modulator.

In some aspects, the present disclosure provides a conjugate of formula (XX), or a pharmaceutically acceptable salt thereof, wherein R¹ is a targeted protein degrader. In some aspects, the present disclosure provides a conjugate of formula (XXXII), or a pharmaceutically acceptable salt thereof, wherein R¹-A′ is a targeted protein degrader. In some aspects, the targeted protein degrader is a substituted isoindoline. In some aspects, the targeted protein degrader is a 5′-substituted isoindoline.

In some aspects, the present disclosure provides a conjugate of formula (XX) or formula (XXXII), or a pharmaceutically acceptable salt thereof, wherein R¹ has the formula:

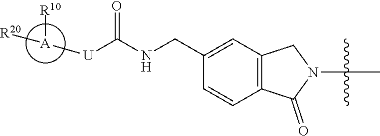

wherein:
[symbol] denotes the point of attachment to the parent molecular moiety;
   A is phenyl or a C₄-C₁₀cycloalkyl ring;
   R¹⁰ is independently selected from hydrogen and halo;
   U is selected from NH and CF₂; and
   R²⁰ is selected from —CH₃, —C(O)R³, —N(R⁴)₂, —(CH₂)ₙOH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
   R³ is hydrogen or C₁-C₆alkyl;
   each R⁴ is independently hydrogen or C₁-C₆alkyl;
   Q′ is O, S, or NR⁴;
   n is 1-6; and
   m is 2-5.

In some aspects:
A is phenyl;
U is NH;
R¹⁰ is halo; and
R²⁰ is methyl.

In some aspects:
A is phenyl;
U is NH;
R¹⁰ is halo; and
R²⁰ is —(CH₂)₂O(CH₂)₂NHCH₃.

In some aspects, the present disclosure provides a conjugate of formula (XX), or a pharmaceutically acceptable salt thereof, wherein R¹ is a proteolysis targeting chimera (PROTAC). In some aspects, the present disclosure provides a conjugate of formula (XXXII), or a pharmaceutically acceptable salt thereof, wherein R¹-A′ is a proteolysis targeting chimera (PROTAC).

In some aspects, the present disclosure provides a conjugate of formula (XX) or formula (XXXII), or a pharmaceutically acceptable salt thereof, wherein R¹ has the formula:

POI-L¹⁰⁰-CBN;

wherein:
POI is a compound that binds to a protein of interest;
L¹⁰⁰ is a PROTAC linker; and
CBN is a cereblon binding moiety.

In some aspects, the protein of interest is a nuclear hormone receptor, a translation termination factor, a transcription factor, a cyclin-dependent kinase, a tyrosine kinase, a serine/threonine kinase, or an E3 ligase. In some aspects, the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

In some aspects, L¹⁰⁰ comprises one or more functional groups selected from glycol, alkyl, alkynyl, triazolyl, piperazinyl, piperidinyl, and combinations thereof.

In some aspects, CBN is selected from

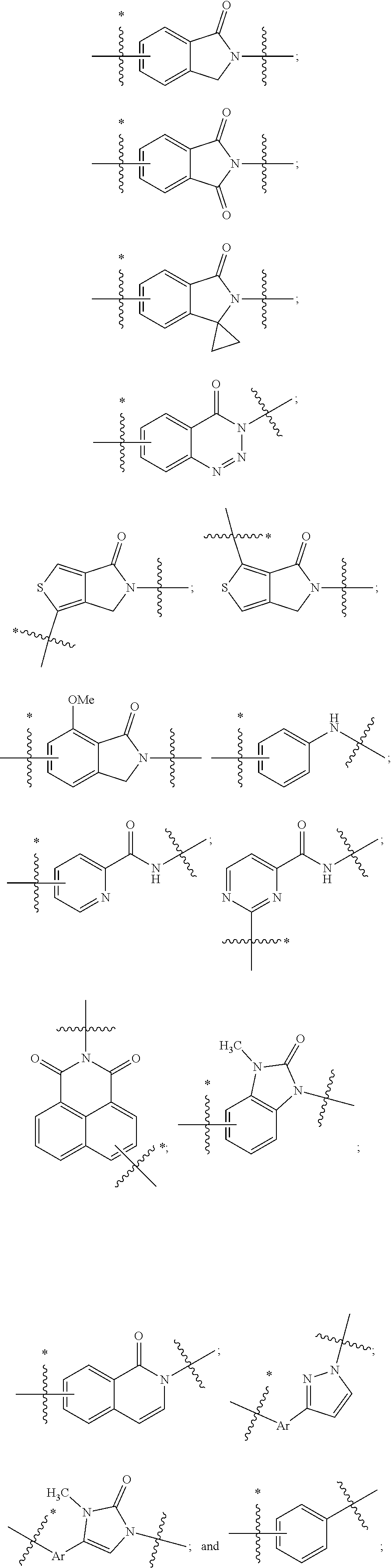

wherein
[symbol] indicates the point of attachment to A′; and
[symbol] indicates the point of attachment to L¹⁰⁰.

In some aspects, the present disclosure provides a conjugate of formula (XX) or formula (XXXII), or a pharmaceutically acceptable salt thereof, wherein R¹ is selected from

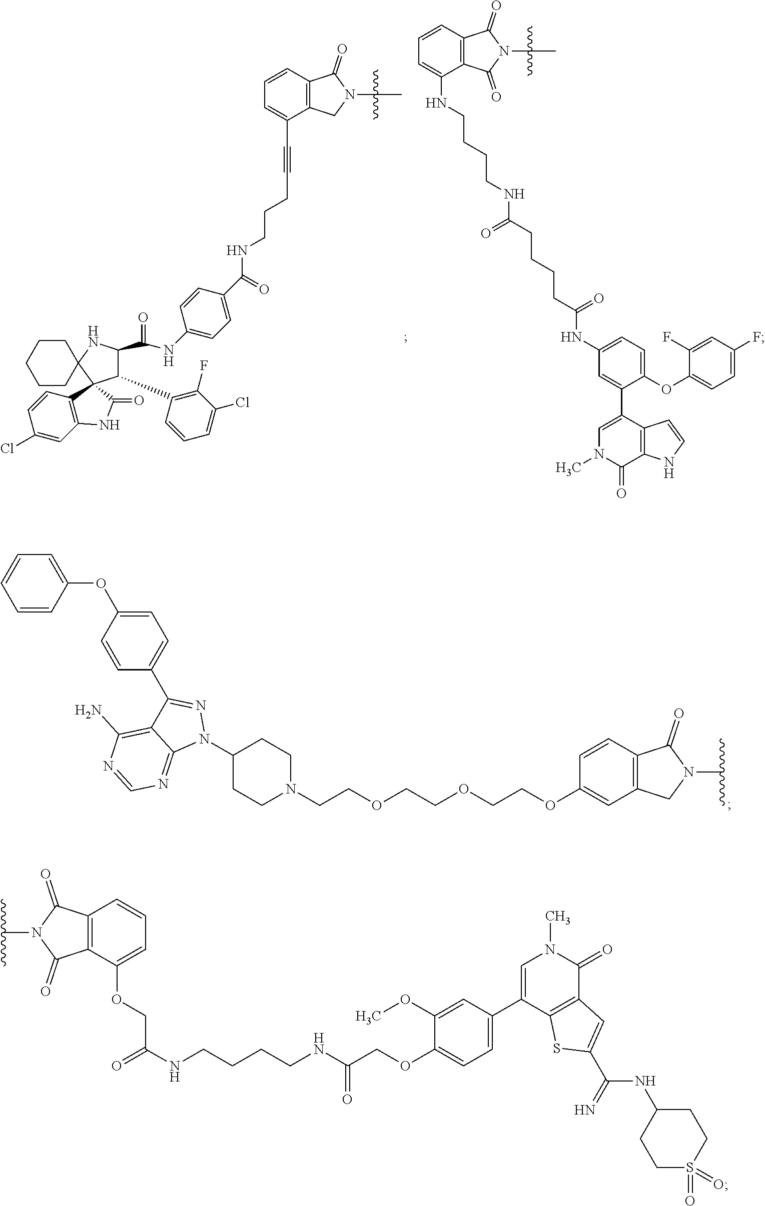

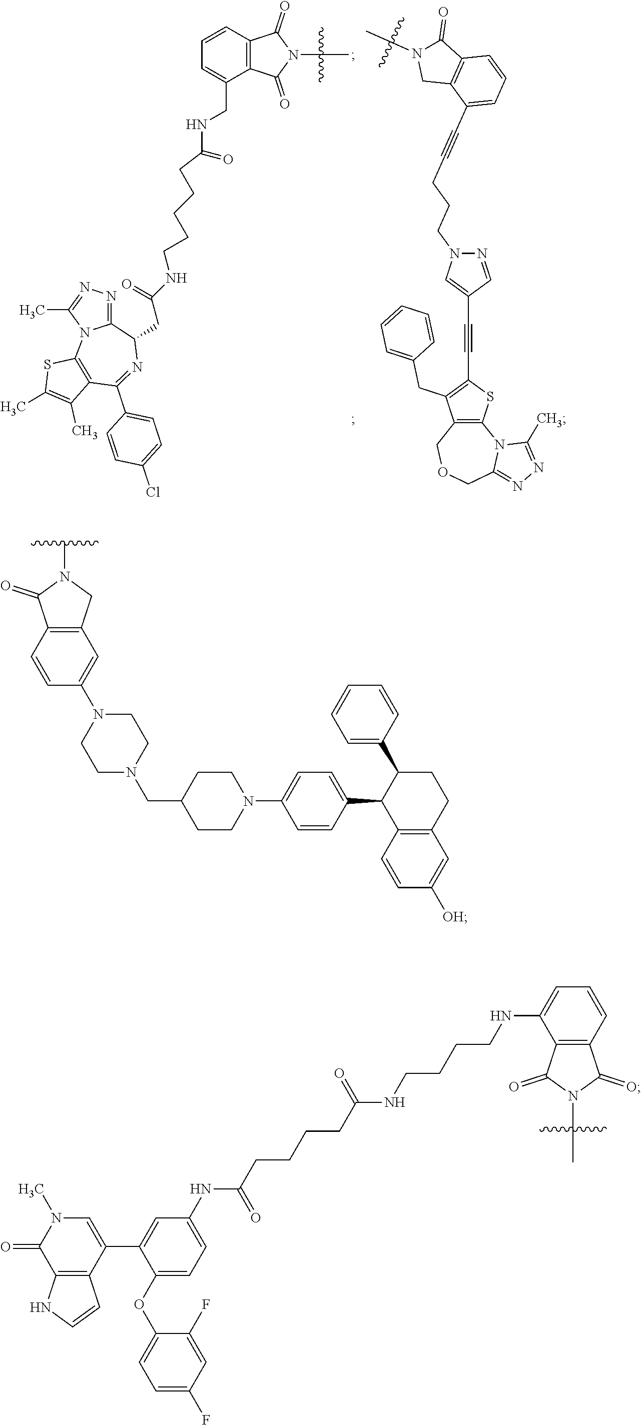

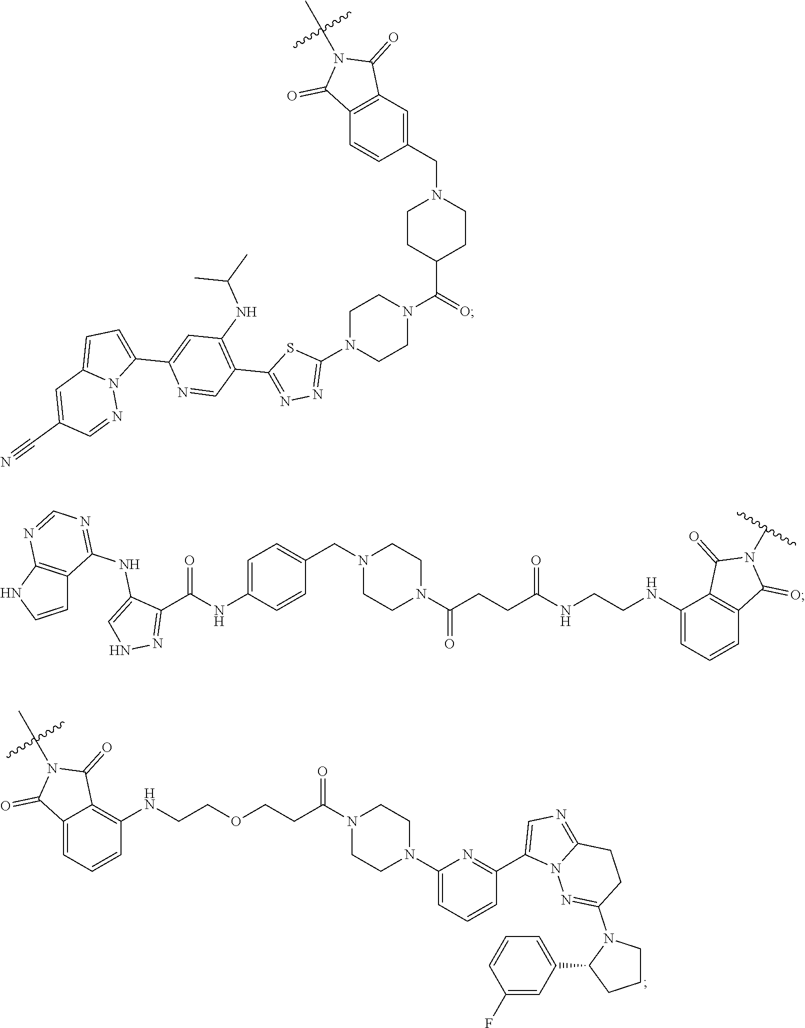

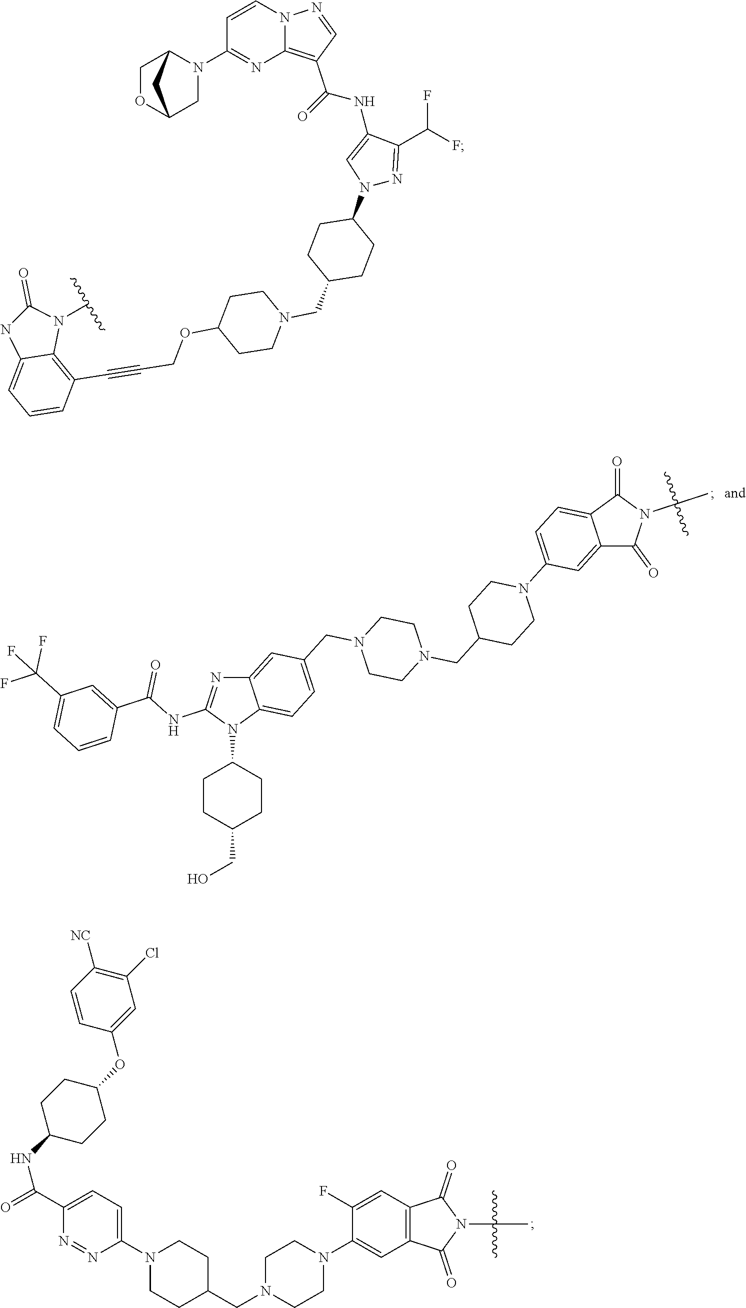

wherein [symbol] denotes the point of attachment to A′.

In some aspects, the present disclosure provides a conjugate of formula (XXI):

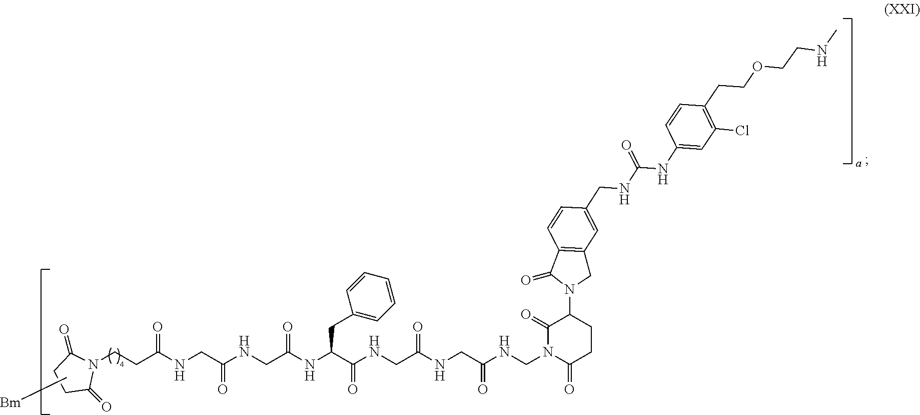

or a pharmaceutically acceptable salt thereof;
wherein:
   a is from 1 to 10; and

Bm is a binding moiety that is capable of specifically binding to a protein. In some aspects, the protein is on a cell surface.

In some aspects, the present disclosure provides a conjugate of formula (XXI), or a pharmaceutically acceptable salt thereof, wherein a is from 2 to 8.

In some aspects, the present disclosure provides a conjugate of formula (XXI), or a pharmaceutically acceptable salt thereof, wherein Bm is an antibody or antigen binding portion thereof.

In some aspects, the present disclosure provides a conjugate of formula (XXI), or a pharmaceutically acceptable salt thereof, wherein the protein that the binding moiety specifically binds to is a surface antigen.

In some aspects, the present disclosure provides a conjugate of formula (XXI), or a pharmaceutically acceptable salt thereof, wherein the surface antigen comprises 5T4, ACE, ADRB3, AKAP-4, ALK, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, c113, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-11Ra, IL-1 receptor, IL-12 receptor, IL-23 receptor, IL-13 receptor, IL-22 receptor, IL-4 receptor, IL-5 receptor, IL-6 receptor, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-1a, LAIR1, LAMP-1, LCK, Legumain, LewisY, LFA-1(CD11a), L-selectin(CD62L), LTLRA2, LIV-1, LMP2, LRRC15, LY6E, LY6K, LY75, MAD-CT-1, MAD-CT-2, MAGE A1, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PD-1, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCR, TEM1/CD248, TEM7R, tenascin C, TF, TGF-1, TGF-β2, TNF-α, TGS5, Tie 2, TIM-1, Tn Ag, TRAC, TRAIL-R1, TRATL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some aspects, the surface antigen comprises HER2, CD20, CD38, CD33, BCMA, CD138, EGFR, FGFR, GD2, PDGFR, or combinations thereof. In some aspects, the surface antigen comprises CD79b. In some aspects, the surface antigen comprises PSMA.

In some aspects, the antibody comprises rituximab, trastuzumab, gemtuzumab, pertuzumab, obinutuzumab, ofatumumab, daratumumab, STI-6129, lintuzumab, huMy9-6, belantamab, indatuximab, cetuximab, dinutuximab, anti-CD38 A2 antibody, huAT15/3 antibody, alemtuzumab, ibritumomab, tositumomab, bevacizumab, panitumumab, tremelimumab, ticilimumab, catumaxomab, oregovomab, or veltuzumab. In some aspects, the antibody comprises lorvotuzumab. In some aspects, the antibody comprises sacituzumab.

In some aspects, the antibody is rituximab, trastuzumab, pertuzumab, huMy9-6, lintuzumab, or gemtuzumab. In some aspects, the antibody is polatuzumab, J591, or belantamab. In some aspects, the antibody is CD33-D.

In some aspects, the present disclosure provides a conjugate of formula (XX) or formula (XXXII), or a pharmaceutically acceptable salt thereof, wherein (a) the protein that the Bm binds to is CD33 and R¹ binds to mouse double minute 2 homolog (MDM2), (b) the protein that the Bm binds to is prostate specific membrane antigen (PSMA) and R¹ binds to androgen receptor (AR), (c) the protein that the Bm binds to is CD33 and R¹ binds to bromodomain-containing protein 4 (BRD4), (d) the protein that the Bm binds to is HER2 and R¹ binds to G1 to S Phase Transition 1 (GSPT1) or (e) the protein that the Bm binds to is CD33 and R¹ binds to GSPT1. In some aspects, the present disclosure provides a conjugate of formula (XX) or formula (XXXII), or a pharmaceutically acceptable salt thereof, wherein the protein that the Bm binds to is CD79b and R¹ binds to IRAK4. In some aspects, the present disclosure provides a conjugate of formula (XX) or formula (XXXII), or a pharmaceutically acceptable salt thereof, wherein the protein that the Bm binds to is HER2 and R¹ binds to BRD4. In some aspects, the present disclosure provides a conjugate of formula (XX) or formula (XXXII), or a pharmaceutically acceptable salt thereof, wherein the protein that the Bm binds to is BCMA and R¹ binds to BRD4. In some aspects, the present disclosure provides a conjugate of formula (XX) or formula (XXXII), or a pharmaceutically acceptable salt thereof, wherein the protein that the Bm binds to is HER2 and R¹ binds to ER.

In some aspects, the present disclosure provides a pharmaceutical composition comprising a conjugate described herein, or a pharmaceutically acceptable salt thereof, and one or more pharmaceutically acceptable carriers.

In some aspects, the present disclosure provides a method of treating cancer in a subject in need thereof, the method comprising administering to the subject a pharmaceutically acceptable amount of a conjugate or composition described herein, or a pharmaceutically acceptable salt thereof. In some aspects, the cancer is a solid tumor. In some aspects, the cancer is a hematologic tumor. In some aspects, the cancer is breast cancer, gastric cancer, lymphoma, acute myeloid leukemia, multiple myeloma, head and neck cancer, squamous cell carcinoma, and/or hepatocellular carcinoma. In some aspects, the cancer is a prostate cancer, breast cancer, gastric cancer, non-small cell lung cancer, bile duct cancer, colon cancer, ovarian cancer, lung cancer, or neuregulin-1 (NRG1)-positive cancer. In some aspects, the cancer is a non-Hodgkin lymphoma (NHL). In some aspects, the cancer is a B-cell non-Hodgkin lymphoma. In some aspects, the cancer is diffuse large B-cell lymphoma (DLBCL). In some aspects, the method further comprises administering to the subject a pharmaceutically acceptable amount of an additional agent prior to, after, or simultaneously with the conjugate or composition described herein, or a pharmaceutically acceptable salt thereof. In some aspects, the additional agent is a cytotoxic agent or an immune response modifier. In some aspects, the immune response modifier is a checkpoint inhibitor. In some aspects, the checkpoint inhibitor comprises a PD-1 inhibitor, a PD-L1 inhibitor, a CTLA-4 inhibitor, a TIM3 inhibitor, and/or a LAG-3 inhibitor.

In some aspects of the method, (a) the protein that the Bm binds to is CD33, R¹ binds to MDM2 or G1 to S Phase Transition 1 (GSPT1), and the cancer is acute myeloid leukemia, (b) the protein that the Bm binds to is prostate specific membrane antigen (PSMA), R¹ binds to androgen receptor (AR), and the cancer is prostate cancer, (c) the protein that the Bm binds to is CD33, R¹ binds to bromodomain-containing protein 4 (BRD4), and the cancer is acute myeloid leukemia or (d) the protein that the Bm binds to is HER2, R¹ binds to G1 to S Phase Transition 1 (GSPT1), and the cancer is breast cancer, gastric cancer, non-small cell lung cancer, bile duct cancer, colon cancer, ovarian cancer, or neuregulin-1 (NRG1)-positive cancer. In some aspects of the method, the protein that the Bm binds to is CD79b, R¹ binds to IRAK4, and the cancer is a non-Hodgkin lymphoma (NHL), e.g., a B-cell non-Hodgkin lymphoma or a diffuse large B-cell lymphoma (DLBCL). In some aspects, the protein that the Bm binds to is HER2, R¹ binds to BRD4, and the cancer is breast cancer, gastric cancer, non-small cell lung cancer, bile duct cancer, colon cancer, ovarian cancer, or neuregulin-1 (NRG1)-positive cancer. In some aspects, the protein that the Bm binds to BCMA, R¹ binds to BRD4, and the cancer is a multiple myeloma.

In some aspects of the method, the method further comprises administering to the subject a pharmaceutically acceptable amount of an additional agent prior to, after, or simultaneously with the conjugate or composition described herein. In some aspects, the additional agent is a cytotoxic agent or an immune response modifier. In some aspects, the immune response modifier is a checkpoint inhibitor

In some aspects, the present disclosure provides a compound of formula (XXII):

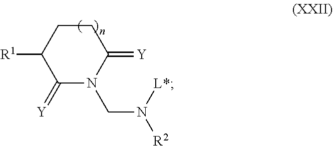

or a pharmaceutically acceptable salt thereof, wherein:
   n is 0 or 1;
   R¹ is a compound that induces a protein-protein interaction;
   R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;
each Y is independently S or O; and
L* is a cleavable linker precursor that conjugates to the binding moiety.

In some aspects, the present disclosure provides a compound of formula (XXXI):

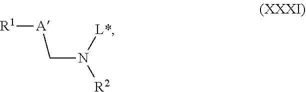

or a pharmaceutically acceptable salt thereof, wherein:
A′ is

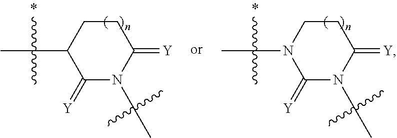

wherein
   n is 0 or 1;
   each Y is independently S or O;
   [symbol] indicates the point of attachment to A′; and
   [symbol] indicates the point of attachment to the methylene group;
   R¹, together with A′, is a compound that induces a protein-protein interaction;
   R² is selected from hydrogen, a group that provides stability to R¹-A′, a group that provides solubility to R¹-A′, and a group that provides stability and solubility to R¹-A′; and
   L is a cleavable linker precursor that conjugates to the binding moiety.

In some aspects, the present disclosure provides a compound of formula (XXII) or formula (XXXI), or a pharmaceutically acceptable salt thereof, wherein L* is a protease cleavable linker precursor.

In some aspects, the present disclosure provides a compound of formula (XXII) or formula (XXXI), or a pharmaceutically acceptable salt thereof, wherein L* is selected from the group consisting of

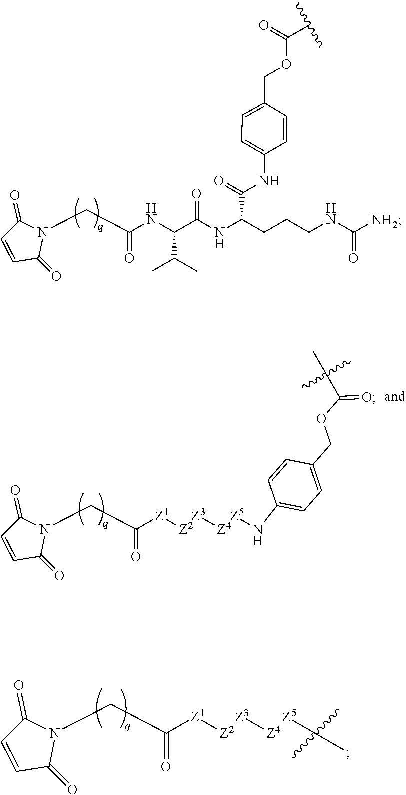

wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues; and
[symbol] is the point of attachment to the parent molecular moiety.

In some aspects, Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

In some aspects:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine;
Z⁴ is selected from the group consisting of L-citrulline, D-citrulline, L-asparagine, D-asparagine, L-lysine, D-lysine, L-phenylalamine, D-phenylalanine, and glycine; and
Z⁵ is absent or glycine.

In some aspects, the present disclosure provides a compound of formula (XXII) or formula (XXXI), or a pharmaceutically acceptable salt thereof, wherein L* is

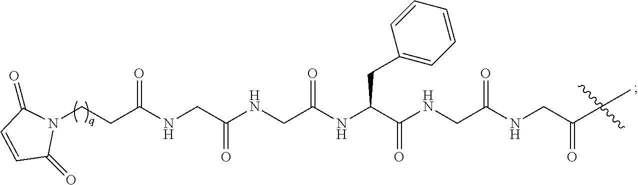

wherein [symbol] is the point of attachment to the parent molecular moiety.

In some aspects, q is 4.

In some aspects, the present disclosure provides a compound of formula (XXII) or formula (XXXI), or a pharmaceutically acceptable salt thereof, L* is a bioreducible linker precursor.

In some aspects, the present provides a compound of formula (XXII) or formula (XXXI), or a pharmaceutically acceptable salt thereof, L* is selected from the group consisting of

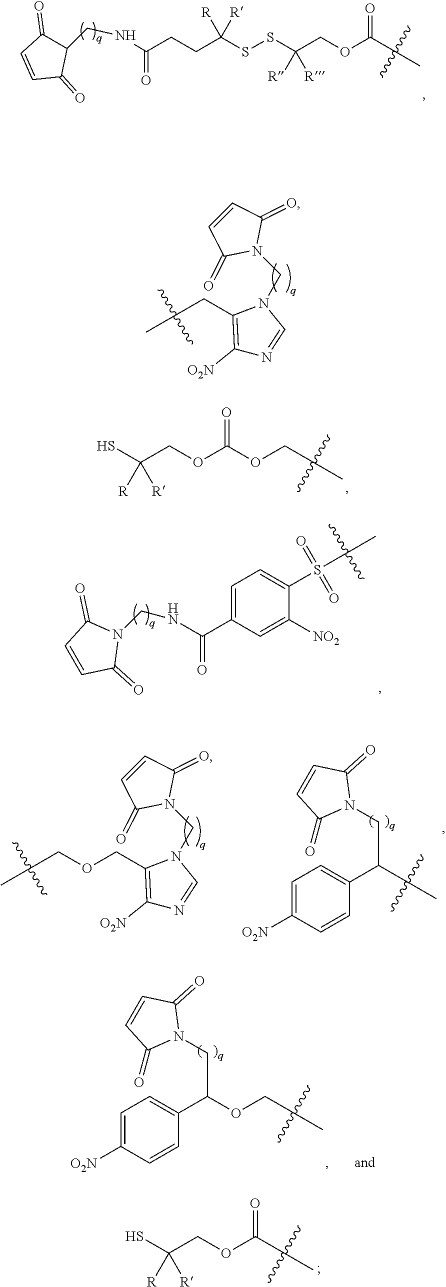

wherein:
q is from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
[symbol] is the point of attachment to the parent molecular moiety.

In some aspects, the present disclosure provides a compound of formula (XXII) or formula (XXXI), or a pharmaceutically acceptable salt thereof, L* is

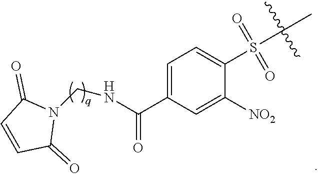

In some aspects, q is 2.

In some aspects, the present disclosure provides a compound of formula (XXII) or formula (XXXI), or a pharmaceutically acceptable salt thereof, wherein L* is a click-to-release linker precursor.

In some aspects, the present disclosure provides a compound of formula (XXII) or formula (XXXI), or a pharmaceutically acceptable salt thereof, wherein L* is

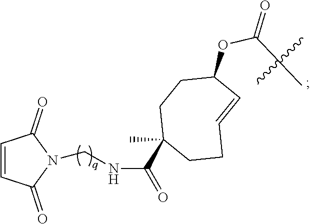

wherein:
   q is from 2 to 10; and
   [symbol] is the point of attachment to the parent molecular moiety.

In some aspects, the present disclosure provides a compound of formula (XXII) or formula (XXXI), or a pharmaceutically acceptable salt thereof, L* is a beta-glucuronidase cleavable linker precursor.

In some aspects, the present disclosure provides a compound of formula (XXII) or formula (XXXI), or a pharmaceutically acceptable salt thereof, wherein L* is

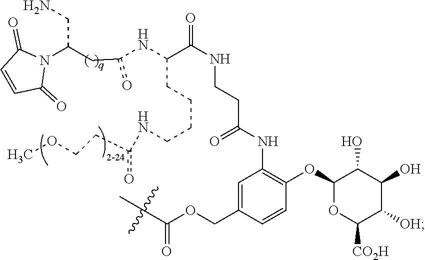

wherein:
   q is from 2 to 10;
   - - - is absent or a bond; and
   [symbol] is the point of attachment to the parent molecular moiety.

In some aspects, the present disclosure provides a compound of formula (XXXI), or a pharmaceutically acceptable salt thereof, wherein R² is a group that provides stability to R¹-A′. In some aspects, R² is selected from C₂-C₆alkenyl, C₁-C₆alkyl; C₂-C₆alkynyl, benzyl, C₃-C₆cycloalkyl, and C₃-C₆cycloalkyl(C₁-C₃alkyl).

In some aspects, the present disclosure provides a compound of formula (XXII) or formula (XXXI), or a pharmaceutically acceptable salt thereof, R² is hydrogen or C₁-C₆alkyl. In some aspects, R² is methyl.

In some aspects, the present disclosure provides a compound of formula (XXXI), or a pharmaceutically acceptable salt thereof, wherein R² is a group that provides solubility to R¹-A′.

In some aspects, R² is selected from:

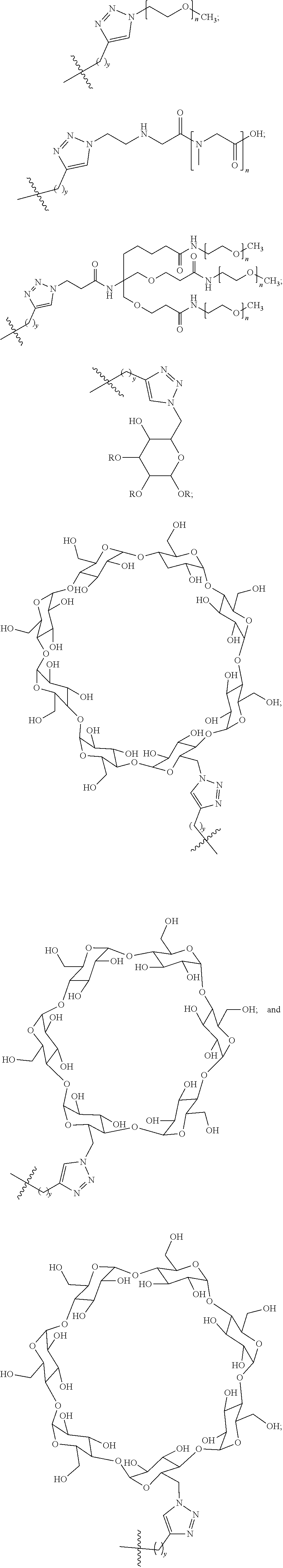

wherein:
each n is independently 1, 2, 3, 4, or 5;
each y is independently 1 or 2;
each R is independently hydrogen, C₆H₁₁O₅, C₁₂H₂₁O₁₀, C₁₈H₃₁O₁₅, or C₂₄H₄₁O₂₀; and

In some aspects, the present disclosure provides a compound of formula (XXII) or formula (XXXI), or a pharmaceutically acceptable salt thereof, wherein each Y is O.

In some aspects, the present disclosure provides a compound of formula (XXXI), or a pharmaceutically acceptable salt thereof, R¹-A′ is a PPI modulator.

In some aspects, the present disclosure provides a compound of formula (XXII), or a pharmaceutically acceptable salt thereof, R¹ is a targeted protein degrader. In some aspects, the present disclosure provides a compound of formula (XXXI), or a pharmaceutically acceptable salt thereof, R¹-A′ is a targeted protein degrader. In some aspects, the targeted protein degrader is a substituted isoindoline. In some aspects, the targeted protein degrader is a 5′-substituted isoindoline.

In some aspects, the present disclosure provides a compound of formula (XXII) or (XXXI), or a pharmaceutically acceptable salt thereof, wherein R¹ is a compound of formula (XXX):

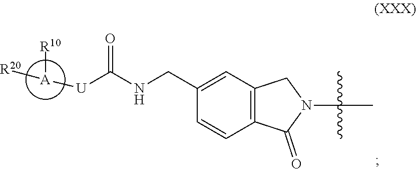

wherein:
[symbol] denotes the point of attachment to the parent molecular moiety;
   A is phenyl or a C₄-C₁₀cycloalkyl ring;
   R¹⁰ is independently selected from hydrogen and halo;
   U is selected from NH and CF₂; and
   R²⁰ is selected from —CH₃, —C(O)R³, —N(R⁴)₂, —(CH₂)ₙOH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
   R³ is hydrogen or C₁-C₆alkyl;
   each R⁴ is independently hydrogen or C₁-C₆alkyl;
   Q′ is O, S, or NR⁴;
   n is 1-6; and
   m is 2-5.

In some aspects,
A is phenyl;
U is NH;
R¹⁰ is halo; and
R²⁰ is methyl.

In some aspects,
A is phenyl;
U is NH;
R¹⁰ is halo; and
R²⁰ is —(CH₂)₂O(CH₂)₂NHCH₃.

In some aspects, the present disclosure provides a compound of formula (XXII), or a pharmaceutically acceptable salt thereof, R¹ is a proteolysis targeting chimera (PROTAC).

In some aspects, the present disclosure provides a compound of formula (XXII), or a pharmaceutically acceptable salt thereof, R¹-A′ is a proteolysis targeting chimera (PROTAC).

In some aspects, R¹ has the formula:

POI-L¹⁰⁰-CBN;
wherein:
   POI is a compound that binds to a protein of interest;
   L¹⁰⁰ is a PROTAC linker; and
   CBN is a cereblon binding moiety.

In some aspects, the protein of interest is a nuclear hormone receptor, a translation termination factor, a transcription factor, a cyclin-dependent kinase, a tyrosine kinase, a serine/threonine kinase, or an E3 ligase. In some aspects, the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

In some aspects, L¹⁰⁰ comprises one or more functional groups selected from glycol, alkyl, alkynyl, triazolyl, piperazinyl, piperidinyl, and combinations thereof.

In some aspects, CBN is selected from

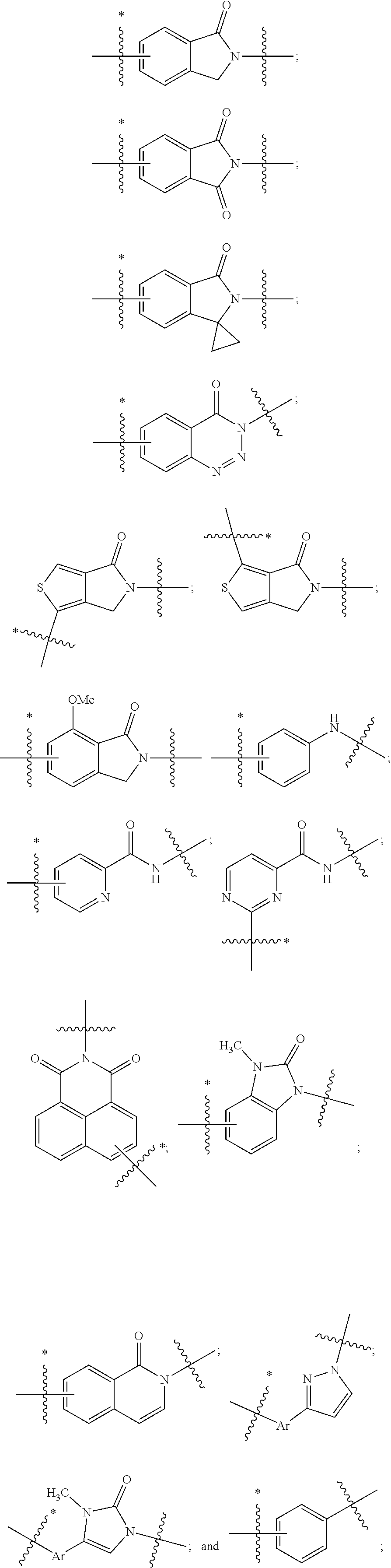

wherein
[symbol] indicates the point of attachment to A′; and
[symbol] indicates the point of attachment to L¹⁰⁰.

In some aspects, the present disclosure provides a compound of formula (XXII) or formula (XXXI), or a pharmaceutically acceptable salt thereof, R¹ is selected from

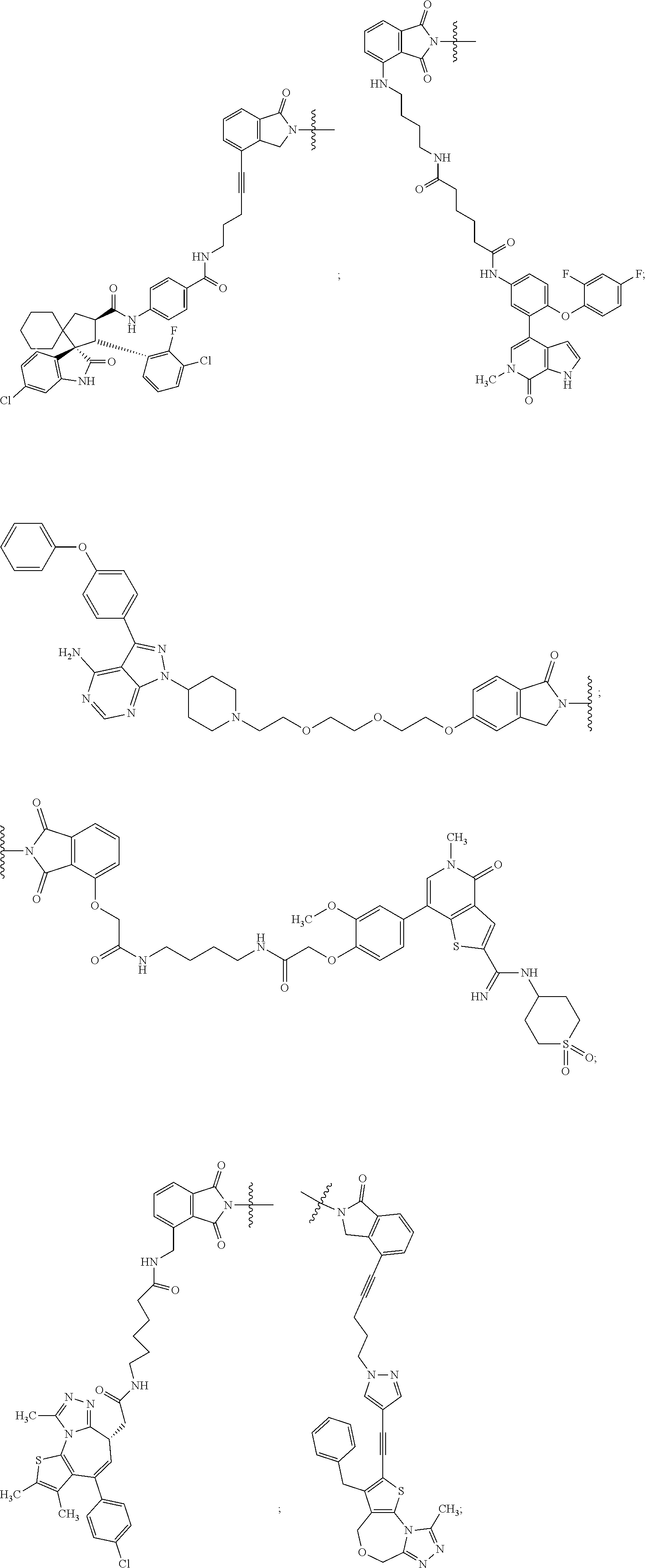

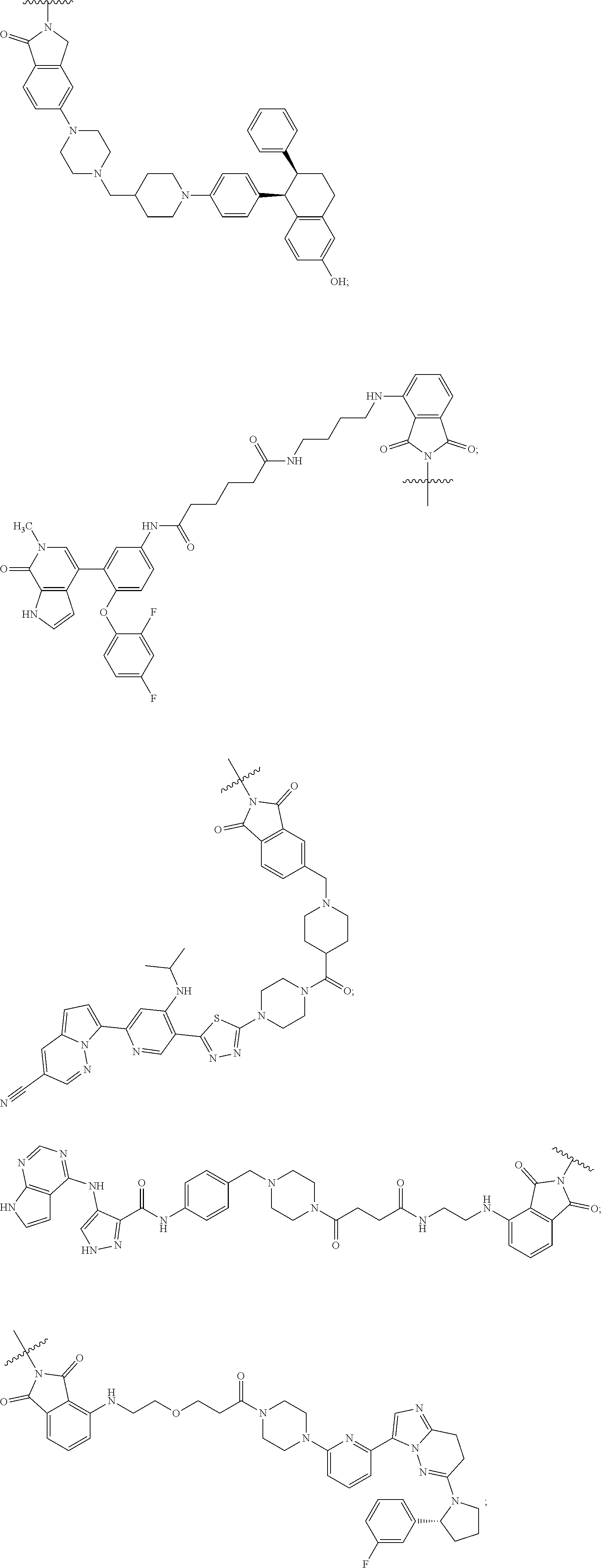

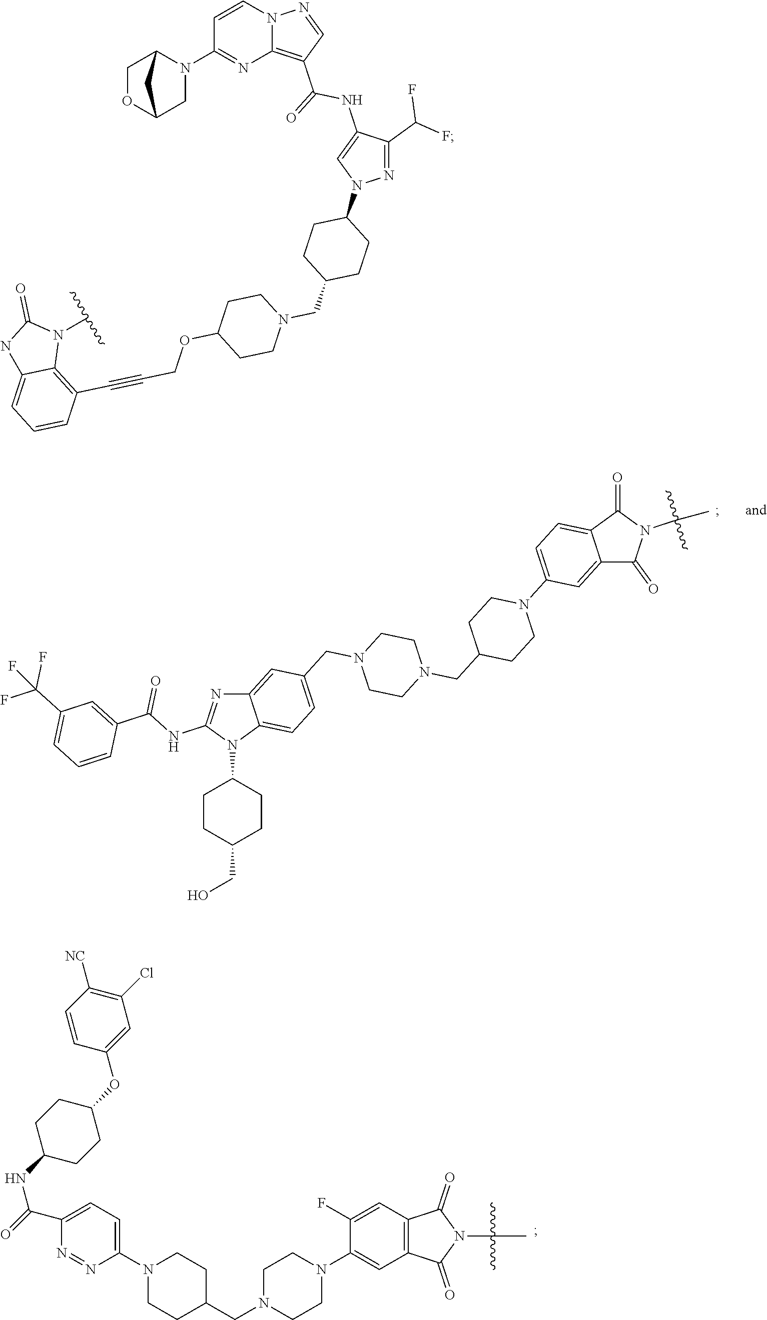

wherein [symbol] denotes the point of attachment to A′.

In certain aspects, the present disclosure provides a method for preparing a conjugate of formula (XXXII):

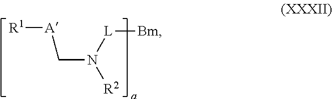

or a pharmaceutically acceptable salt thereof, wherein:
   a is from 1 to 10:
      A′ is

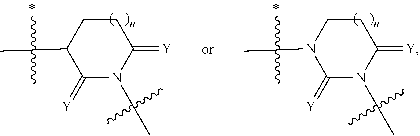

wherein
n is 0 or 1;
each Y is independently S or O;
[symbol] indicates the point of attachment to R¹; and
[symbol] indicates the point of attachment to the methylene group;
R¹, together with A′, is a compound that induces a protein-protein interaction;
R² is selected from hydrogen, a group that provides stability to R¹-A′, a group that provides solubility to R¹-A, and a group that provides stability and solubility to R¹-A′;
L is a cleavable linker; and
Bm is a binding moiety that is capable of specifically binding to a protein; the method comprising:
reacting a compound of (XXXI)

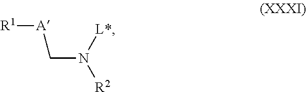

or a pharmaceutically acceptable salt thereof, wherein:
   A′, R¹, and R² are as defined above and
   L* is a cleavable linker precursor;
with a binding moiety that is capable of specifically binding to a protein.

In some aspects, L* to a cysteine, lysine, tyrosine, or glutamine in the Bm. In some aspects, the cysteine or lysine is an engineered cysteine or lysine. In some aspects, the cysteine or lysine is endogenous to the Bm.

In some aspects, the binding moiety is an antibody or an antigen binding portion thereof. In some aspects, L* is attached to an engineered cysteine at heavy chain position S239 and/or K334 of the antibody or antigen binding portion thereof according to EU numbering. In some aspects, L* is attached to the glutamine at heavy chain position 295 of the antibody or antigen binding portion thereof according to EU numbering. In some aspects, the attaching is via site-specific conjugation.

In some aspects of the method, (a) the protein that the Bm binds to is CD33 and R¹ binds to mouse double minute 2 homolog (MDM2), (b) the protein that the Bm binds to is prostate specific membrane antigen (PSMA) and R¹ binds to androgen receptor (AR), (c) the protein that the Bm binds to is CD33 and R¹ binds to bromodomain-containing protein 4 (BRD4), (d) the protein that the Bm binds to is HER2 and R¹ binds to G1 to S Phase Transition 1 (GSPT1), or (e) the protein that the Bm binds to is CD33 and R¹ binds to GSPT1. In some aspects of the method, the protein that the Bm binds to is CD79b and R¹ binds to IRAK4. In some aspects of the method, the protein that the Bm binds to is HER2 and R¹ binds to BRD4. In some aspects of the method, the protein that the Bm binds to is BCMA and R¹ binds to BRD4. In some aspects of the method, the protein that the Bm binds to is HER2 and R¹ binds to ER.

In some aspects of the method, R¹-A′ is a targeted protein degrader. In some aspects, R¹ has the formula:

POI-L¹⁰⁰-CBN;
wherein:
   POI is a compound that binds to a protein of interest;
   L¹⁰⁰ is a PROTAC linker; and
   CBN is a cereblon binding moiety.

In some aspects, the protein of interest is a nuclear hormone receptor, a translation termination factor, a transcription factor, a cyclin-dependent kinase, a tyrosine kinase, a serine/threonine kinase, or an E3 ligase. In some aspects, the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

In some aspects, L¹⁰⁰ comprises one or more functional groups selected from glycol, alkyl, alkynyl, triazolyl, piperazinyl, piperidinyl, and combinations thereof.

In some aspects, CBN is selected from:

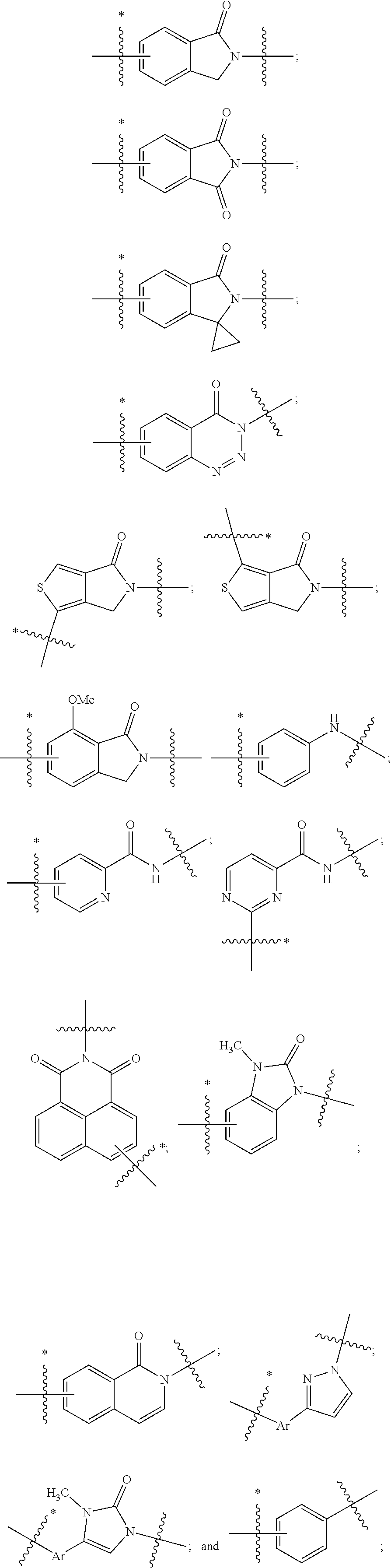

wherein
[symbol] indicates the point of attachment to A′; and
[symbol] indicates the point of attachment to L¹⁰⁰.

In some aspects, R¹ is selected from:

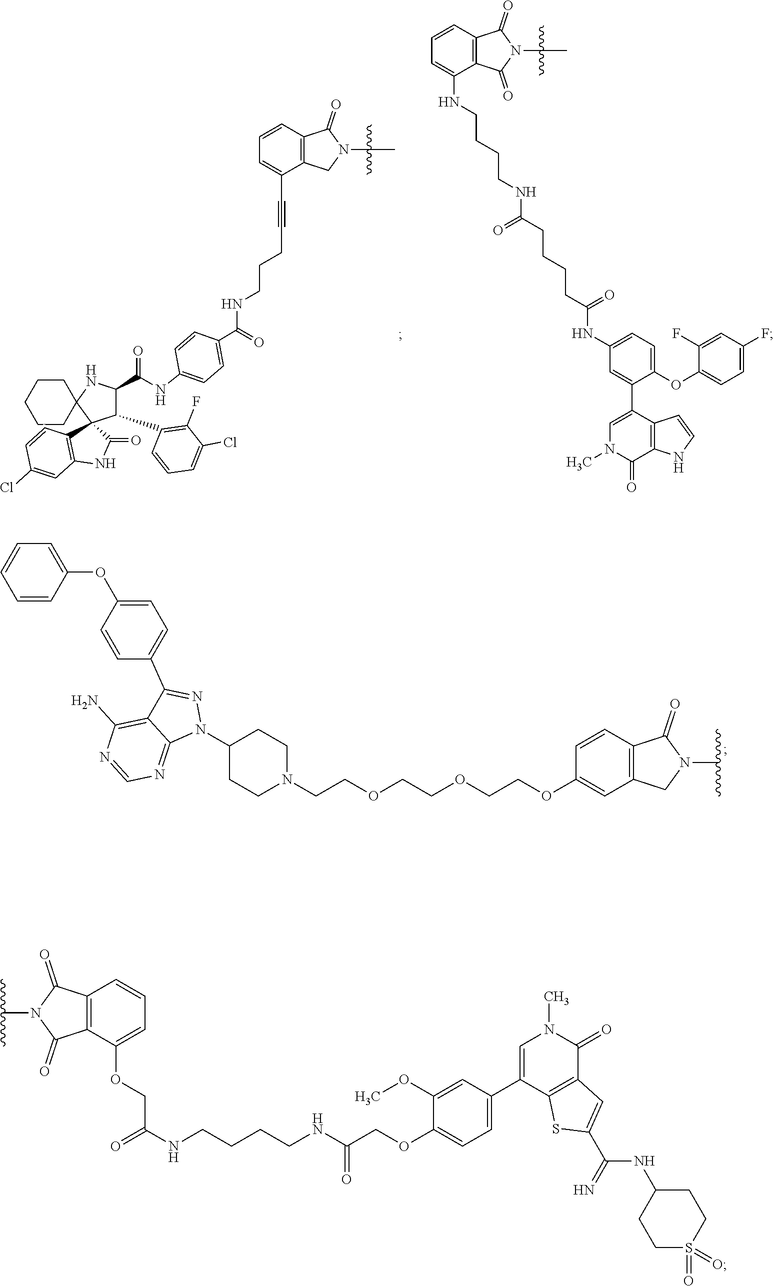

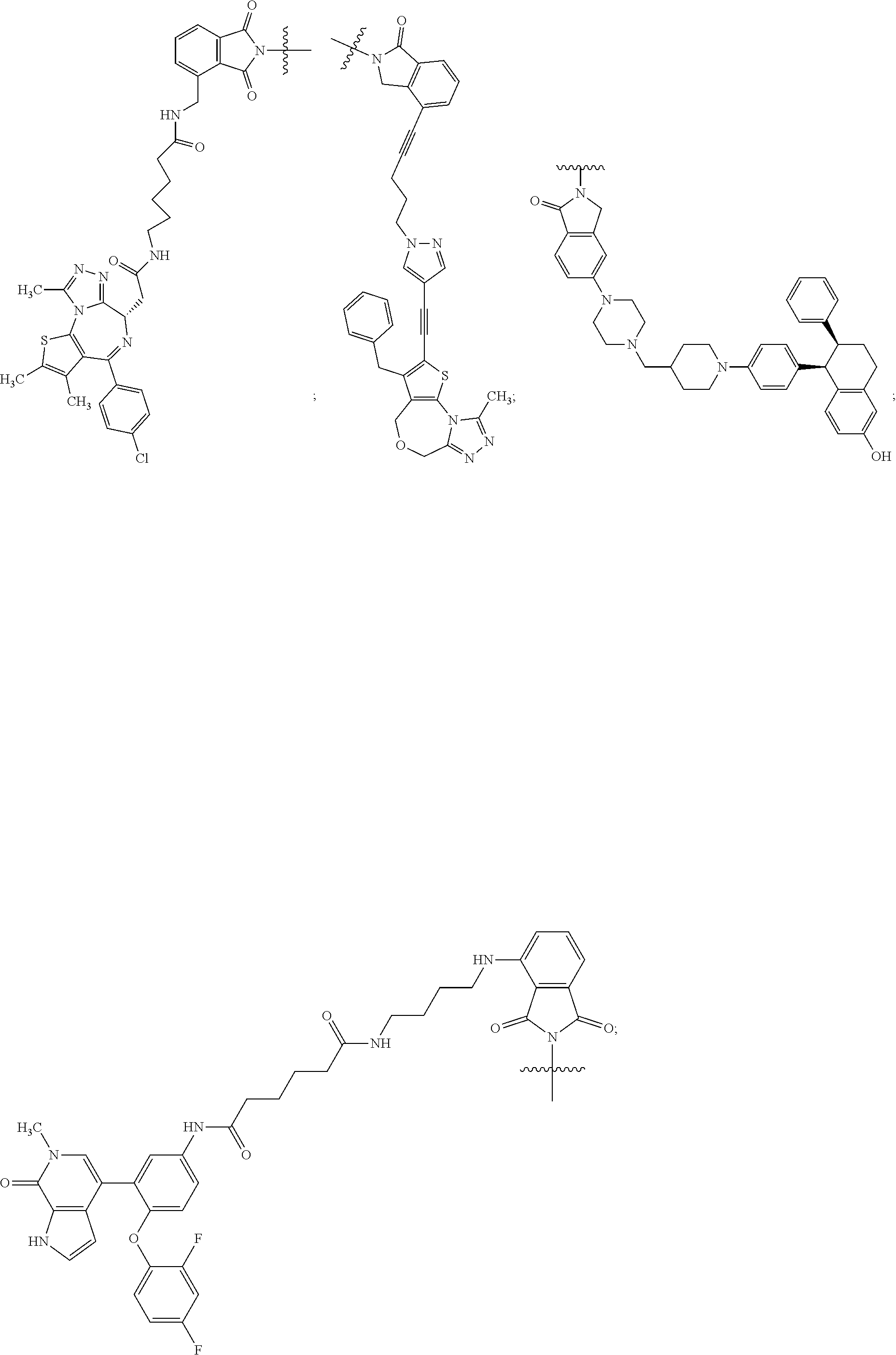

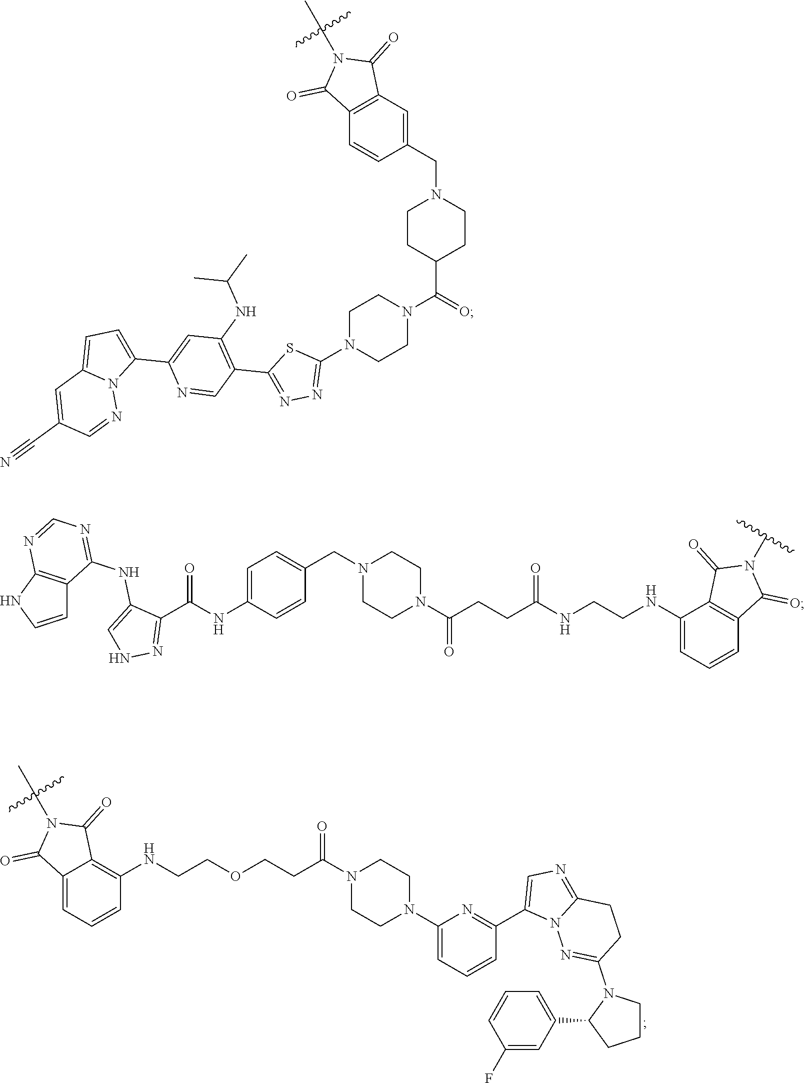

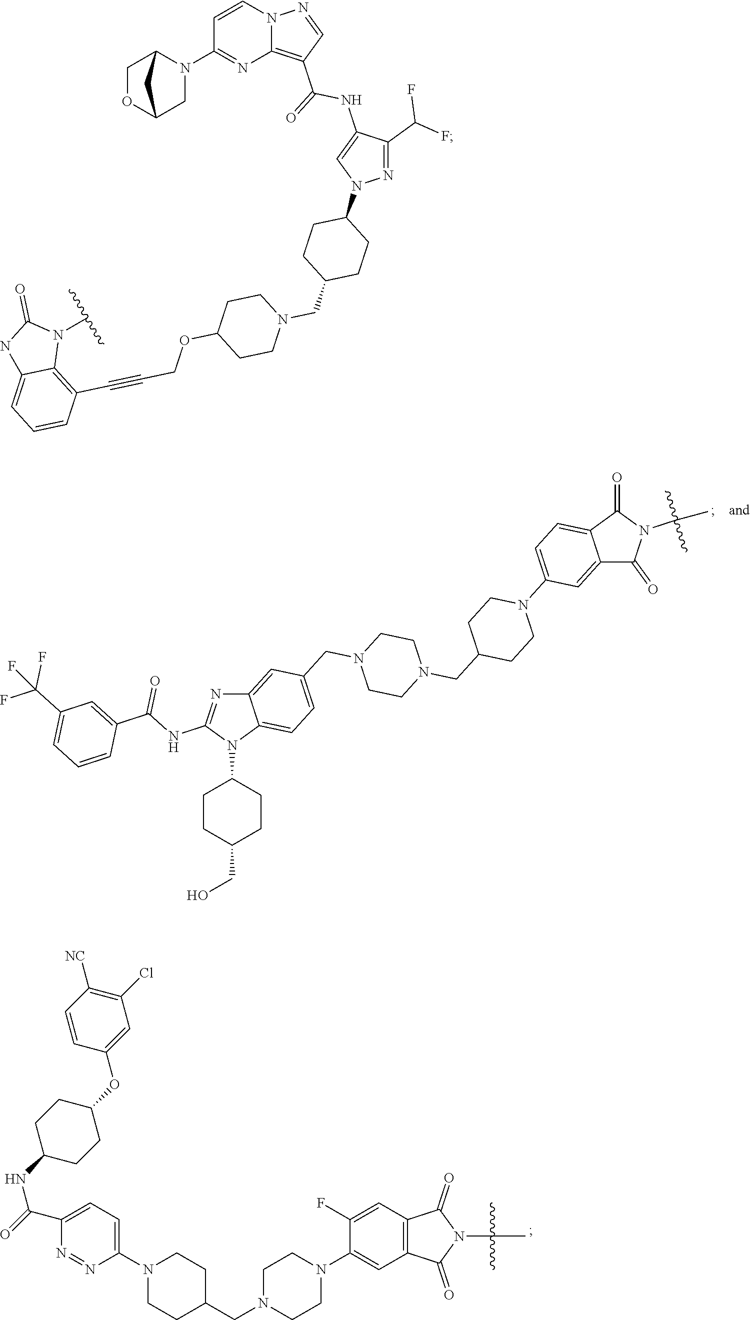

wherein [symbol] denotes the point of attachment to A′.

In some aspects, the present disclosure provides a conjugate made by the methods described herein.

In some aspects, the present disclosure provides a method of delivering a conjugate that induces a protein-protein interaction to a cell, the method comprising contacting the cell with a conjugate or composition described herein, or a pharmaceutically acceptable salt thereof.

In some aspects, the present disclosure provides a method of delivering a conjugate of formula (XXXII):

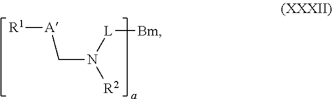

or a pharmaceutically acceptable salt thereof to a cell, wherein:
a is from 1 to 10;
   A′ is

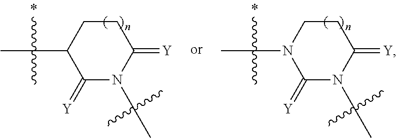

wherein
n is 0 or 1;
each Y is independently S or O;
[symbol] indicates the point of attachment to R1; and
[symbol] indicates the point of attachment to the methylene group;
R1, together with A′, is a compound that induces a protein-protein interaction;
R2 is selected from hydrogen, a group that provides stability to R1-A′, a group that provides solubility to R1-A′, and a group that provides stability and solubility to R1-A′; L is a cleavable linker; and
Bm is a binding moiety that is capable of specifically binding to a protein;
the method comprising contacting the cell with a conjugate of formula (XXXII), or a pharmaceutically acceptable salt thereof.

### BRIEF DESCRIPTION OF THE FIGURES

FIG. 1 depicts LCMS spectra of a pertuzumab-Compound (I) conjugate (DAR=8) at 4° C. at pH 5.5 and after incubation for 24 h at 37° C., pH 7.5.

FIGS. 2A and 2B depict LCMS spectra of a pertuzumab-Compound (VIII) conjugate (DAR=8) after incubation for 24 h at 37° C., pH 7.4.

FIG. 3 depicts an LCMS spectra of a pertuzumab-Compound (X) conjugate (DAR=3.66) after incubation for 24 h at 37° C., pH 7.5.

FIG. 4 depicts an LCMS spectra of a pertuzumab-Compound (XI) conjugate (DAR=3.74) after incubation for 24 h at 37° C., pH 7.5.

FIG. 5 depicts the RP-LC-UV profile of pertuzumab-Compound (XI) conjugate (DAR=3.74) before and after treatment with cysteine protease papain and of neoDegrader P1 and also depicts the LCMS of the conjugate before and after treatment with cysteine protease papain.

FIG. 6 depicts in vitro activity of representative conjugates against BT-474 breast cancer cell line. The X axis shows log antibody concentration (M). The Y axis shows % viability of the BT-474 cells when treated with pertuzumab-Compound (X) conjugate (triangle, solid line) and rituximab-Compound (X) conjugate (circle, dotted line).

FIG. 7 depicts in vitro activity of representative conjugates against NCI-N87 cancer cell line. The X axis shows log antibody concentration (M). The Y axis shows % viability of the NCI-87 cells when treated with pertuzumab-Compound (X) conjugate (triangle, solid line) and rituximab-Compound (X) conjugate (circle, dotted line).

FIG. 8 depicts in vitro activity of representative conjugates against BT-474 breast cancer cell line. The X axis shows log antibody concentration (M). The Y axis shows % viability of the BT-474 cells when treated with pertuzumab-Compound (XI) conjugate (triangle) and rituximab-Compound (XI) conjugate (circle).

FIG. 9 depicts in vitro activity of representative conjugates against NCI-H929 cancer cell line. The X axis shows log antibody concentration (M). The Y axis shows % viability of the NCI-H929 cells when treated with belantamab-Compound (XIV) conjugate (circle), synagis-Compound (XIV) conjugate (square), belantamab (upright triangle), and unconjugated Compound (XIII) (downward triangle).

FIG. 10 depicts in vitro activity of a representative conjugate against Jurkat HiBiT labeled cells (HER2−) in the absence and presence of SK-BR-3 cells (HER2+). The X axis shows the log payload concentration (M). The Y axis shows % viability of the Jurkat HiBiT cells in the presence (circle, solid line) and absence (square, dotted line) of SK-BR-3 cells when treated with pertuzumab-Compound (X) conjugate.

FIG. 11 depicts the in vitro activity of representative conjugates against BT-474 breast cancer cell line. The X axis shows log antibody concentration (M). The Y axis shows % viability of the BT-474 cells when treated with pertuzumab-Compound (XVIII) conjugate (circle), pertuzumab-Compound (XI) conjugate (square), and rituximab-Compound (XVIII) conjugate (triangle).

FIG. 12 depicts the in vitro activity of representative conjugates against BT-474 breast cancer cell line. The X axis shows log antibody concentration (M). The Y axis shows % viability of the BT-474 cells when treated with pertuzumab-Compound (XLI) conjugate (circle), pertuzumab-Compound (Ie) conjugate (square), pertuzumab-Compound (XIX) conjugate (upward triangle), pertuzumab-Compound (Ii) conjugate (downward triangle), pertuzumab-Compound (Ia) conjugate (diamond), rituximab-Compound (XLI) conjugate (hexagon), and rituximab-Compound (XIX) conjugate (star).

FIG. 13 depicts the in vitro activity of representative conjugates against MV-411 AML cell line. The X axis shows log antibody concentration (M). The Y axis shows % viability of the BT-474 cells when treated with gemtuzumab-Compound (XL) conjugate (circle), gemtuzumab-Compound (XL) conjugate plus gemtuzumab (square), gemtuzumab-Compound (XL) conjugate plus trastuzumab (upward triangle), Compound 40-3 (downward triangle), gemtuzumab (diamond), and trastuzumab (star).

FIG. 14 depicts the degradation of BRD4 protein by Compound (XL) and Compound 40-3.

FIG. 15A depicts the change in HCC1569 (breast cancer) tumor volume over time when treated with 3 mg/kg or 10 mg/kg of pertuzumab-Compound (Ia) (square and upward triangle, respectively), and with 3 mg/kg or 10 mg/kg of pertuzumab Compound (XI) (downward triangle and diamond, respectively).

FIG. 15B depicts the change in body weight of mice with in HCC1569 (breast cancer) tumors over time when treated with 3 mg/kg or 10 mg/kg of pertuzumab-Compound (Ia) (square and upward triangle, respectively), and with 3 mg/kg or 10 mg/kg of pertuzumab Compound (XI) (downward triangle and diamond, respectively).

FIG. 16 depicts an SEC chromatogram of an anti-PSMA antibody with a Cys-mutation-Compound (XV) endogenous cysteine conjugate with DAR of 4.

FIG. 17 depicts an SEC chromatogram of a J591 antibody with a S239C mutation-Compound (XV) site-specific engineered cysteine conjugate with DAR of 1.85.

FIG. 18 depicts the HPLC chromatogram of Compound (XIX).

FIG. 19A depicts the HPLC chromatogram of the reaction mixture when Compound (XIX) is treated with cysteine. Compound (XIX) was completely consumed, and the sole identified product had a retention time of 2.41 minutes.

FIG. 19B depicts the MS data of the peak at retention time 2.4 minutes, which corresponds to Compound 18-6.

FIG. 20 depicts an SEC chromatogram of a HER2-A antibody (wild type sequence)-Compound (XLII) conjugate with a DAR of 3.3.

FIG. 21 depicts an SEC chromatogram of a HER2-A antibody (containing a cysteine mutant for site specific conjugation)-Compound (XLII) conjugate with a DAR of 2.0.

FIG. 22 depicts the change in MV-4-11 tumor volume over time when treated with 10 mg/kg of CD33-D antibody-Compound (XL) conjugate (square), 0.4 mg/kg of BRD4 heterobifunctional degrader small molecule (Compound 15 from Xiamg, W. et al., Biorganic Chemistry 2021, volume 115) (upward triangle), 10 mg/kg ARV-825 (downward triangle), and vehicle (circle).

FIG. 23 depicts individual tumor volumes over time for each group depicted in FIG. 22.

FIG. 24 depicts the change in body weight over time in the mice used in the study depicted in FIG. 22.

FIG. 25 depicts CD79b binding affinity of CD79b-A antibody-Compound (XLIII) conjugates having three different DARs (closed circle, upward triangle, and diamond), Synagis N297A (open circle), and CD79b-A antibody (square).

FIG. 26 depicts a Western blot showing the degradation of IRAK4 (as compared to a p-actin control) by CD79b-A antibody-Compound (XLIII) conjugate, unconjugated payload, and unconjugated CD79b-A antibody.

FIG. 27 depicts a Western blot showing the amount of IRAK4 (as compared to a 3-actin control) in the presence of CD79b-A antibody-Compound (XLIII) conjugate, unconjugated CD79b-A antibody, or both CD79b-A antibody-Compound (XLIII) conjugate and unconjugated CD79b-A antibody.

### DETAILED DESCRIPTION

The present disclosure is directed to a conjugate of formula (XX):

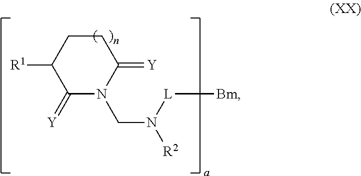

or a pharmaceutically acceptable salt thereof, wherein:
a is from 1 to 10;
n is 0 or 1;
R¹ is a compound that induces a protein-protein interaction;
R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;
each Y is independently S or 0;
L is a cleavable linker; and
Bm is a binding moiety that is capable of specifically binding to a protein. In some aspects, the protein is on a cell surface.

In some aspects, R² is methyl.

The present disclosure is further directed to a conjugate of formula (XXXII):

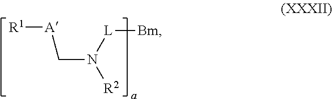

or a pharmaceutically acceptable salt thereof, wherein:
a is from 1 to 10;
A′ is

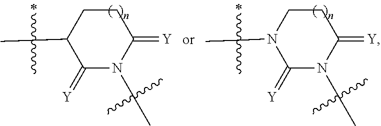

or Y wherein
   n is 0 or 1;
   each Y is independently S or O;
   [symbol] indicates the point of attachment to R¹; and
   [symbol] indicates the point of attachment to the methylene group;
R¹, together with A′, is a compound that induces a protein-protein interaction;
R² is selected from hydrogen, a group that provides stability to R¹-A′, a group that provides solubility to R¹-A′, and a group that provides stability and solubility to R¹-A′;
L is a cleavable linker; and
Bm is a binding moiety that is capable of specifically binding to a protein. In some aspects, the protein is on a cell surface.

The present disclosure also provides compositions comprising the conjugates, methods of using the conjugates, and the compounds above that are conjugated to the binding moiety.

Including a spacer capable of undergoing the retro-Mannich reaction described herein is a suitable general solution to linking and releasing a gluturamide or dihydrouracil containing degrader to an antibody or other cell binding agent. By using this technology, no additional chemical handle needs to be introduced to effect antibody based delivery to cancer cells.

### I. Definitions

In order that the present description can be more readily understood, certain terms are first defined. Additional definitions are set forth throughout the detailed description.

It is to be noted that the term “a″ or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The terms “targeted protein degrader,” and “neoDegrader,” as used herein, refer to a molecule that forms a ternary complex with an E3 ubiquitin ligase which is capable of targeting a protein for degradation. Examples include, but are not limited to, molecular glues and PROTACs. Examples of molecular glues include, but are not limited to CC-90009, lenalidomide, pomalidomide, DKY709, and Compound P1 described in WO2021/198965.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “single domain antibody,” also known as a nanobody, is an antibody fragment consisting of a single monomeric variable antibody domain with a molecular weight of from about 12 kDa to about 15 kDa. Single body antibodies can be based on heavy chain variable domains or light chains. Examples of single domain antibodies include, but are not limited to, V_{H}H fragments and V_{NAR} fragments.

“Antibody fragments” comprise a portion of an intact antibody, generally the antigen binding or variable region thereof. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

An “intact antibody” is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g., Old World Monkey, Ape etc.) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called .alpha., .delta., .epsilon., .gamma., and .mu., respectively. The subunit structures and three-dimensional configurations of different classes of immunoglobulins are well known.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the terms “linking” and “conjugating” are used interchangeably and each refer to the covalent or non-covalent attachment of two or more moieties comprising one or more compounds that induce protein-protein interaction and a binding moiety. In some aspects the linking or conjugating can comprise a linker.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody. An exemplary FcR is a native sequence human FcR. Moreover, a FcR may be one which binds an IgG antibody (a gamma receptor) and includes receptors of the Fc.gamma.RI, Fc.gamma.RII, and Fc.gamma. RIII subclasses, including allelic variants and alternatively spliced forms of these receptors. Fc.gamma.RII receptors include Fc.gamma.RIIA (an “activating receptor”) and Fc.gamma.RIIB (an “inhibiting receptor”), which have similar amino acid sequences that differ primarily in the cytoplasmic domains thereof. Activating receptor Fc.gamma.RIIA contains an immunoreceptor tyrosine-based activation motif (ITAM) in its cytoplasmic domain. Inhibiting receptor Fc.gamma.RIIB contains an immunoreceptor tyrosine-based inhibition motif (ITIM) in its cytoplasmic domain. Other FcRs, including those to be identified in the future, are encompassed by the term “FcR” herein. The term also includes the neonatal receptor, FcRn, which is responsible for the transfer of maternal IgGs to the fetus.

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta.-sheet configuration, connected by three hypervariable regions, which form loops connecting, and in some cases forming part of, the beta.-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (.kappa.) and lambda (.lamda.), based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework. These CDR-grafted antibodies generally do not retain their original affinity for antigen binding, and in fact, affinity is often severely impaired. Besides the CDRs, select non-human antibody framework residues must also be incorporated to maintain proper CDR conformation. The transfer of key mouse framework residues to the human acceptor in order to support the structural conformation of the grafted CDRs has been shown to restore antigen binding and affinity. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain aspects, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., a conjugate disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### II. Protein-Protein Interaction Inducers

The present disclosure provides conjugates of formula (XX):

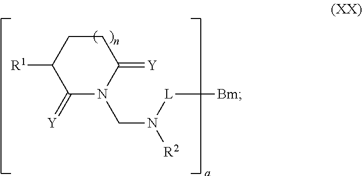

or a pharmaceutically acceptable salt thereof, wherein R¹ is a compound that induces a protein-protein interaction.

The present disclosure further provides conjugates of formula (XXXII):

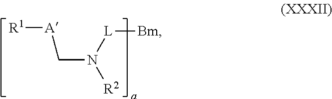

or a pharmaceutically acceptable salt thereof, wherein:
A′ is

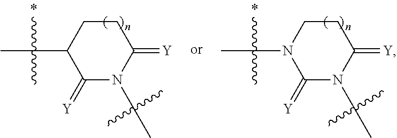

wherein
   n is 0 or 1;
   each Y is independently S or O;
   [symbol] indicates the point of attachment to R¹; and
   [symbol] indicates the point of attachment to the methylene group; and
R¹, together with A′, is a compound that induces a protein-protein interaction.

Compounds that induce protein-protein interactions include protein-protein interaction modulators such as those described in Biophysical Reviews 2019, 11: 559-581.

In certain aspects, the protein-protein interaction inducers comprise targeted protein degraders, which can disassemble and break down undesired proteins.

In some aspects, the targeted protein degraders comprise substituted isoindole compounds. In some aspects, the targeted protein degraders comprise 5′-substituted isoindole compounds. In certain aspects, R¹ is a compound of formula (XXX) shown below:

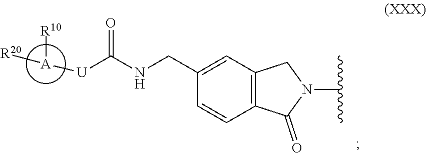

wherein:
[symbol] is the point of attachment to the parent molecular moiety;
A is phenyl or a C₄-C₁₀cycloalkyl ring;
U is selected from NH and CF₂;
R¹⁰ is independently selected from hydrogen and halo;
R²⁰ is selected from —CH₃, —C(O)R³, —N(R⁴)₂, —(CH₂)ₙOH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
   R³ is hydrogen or C₁-C₆alkyl;
   each R⁴ is independently hydrogen or C₁-C₆alkyl;
   Q′ is O, S, or NR⁴;
   n is 1-6; and
   m is 2-5.

In certain aspects, the present disclosure provides compounds of formula (XXX), or pharmaceutically acceptable salts thereof, wherein:
A is a phenyl ring or a C₄-C₁₀cycloalkyl ring;
U is NH;
R¹⁰ is selected from hydrogen and halo;
R²⁰ is selected from —(CH₂)ₙQ′(CH₂)ₘN(R⁴)₂, —(CH₂)ₙOH, —N(R⁴)₂, and —C(O)R³;
   wherein:
m is 2;
n is 2;
Q′ is —O—;
R³ is methyl; and
each R⁴ is independently selected from hydrogen and methyl.

As used herein, the term “C₁-C₆alkoxy,” as used herein, refers to a C₁-C₆alkyl group attached to the parent molecular moiety through an oxygen atom.

As used herein, the term “C₁-C₆alkoxyC₁-C₆alkyl” refers to a C₁-C₆alkoxy group attached to the parent molecular moiety through a C₁-C₆alkyl group.

As used herein, the term “C₁-C₆alkyl” refers to a group derived from a straight or branched chain saturated hydrocarbon containing from one to six carbon atoms.

As used herein, the term “C₄-C₁₀cycloalkyl” refers to a a saturated monocyclic, hydrocarbon ring system having four to ten carbon atoms and zero heteroatoms. Representative examples of cycloalkyl groups include, but are not limited to, cyclobutyl, cyclopentyl, and cyclohexyl. The cycloalkyl groups containing between seven and ten atoms may be monocyclic or fused, spirocyclic, or bridged bicyclic structures.

As used herein, the term “halo” refers to F, Cl, Br, or I.

In some aspects, the compound of formula (XXX) is a compound selected from the group consisting of:

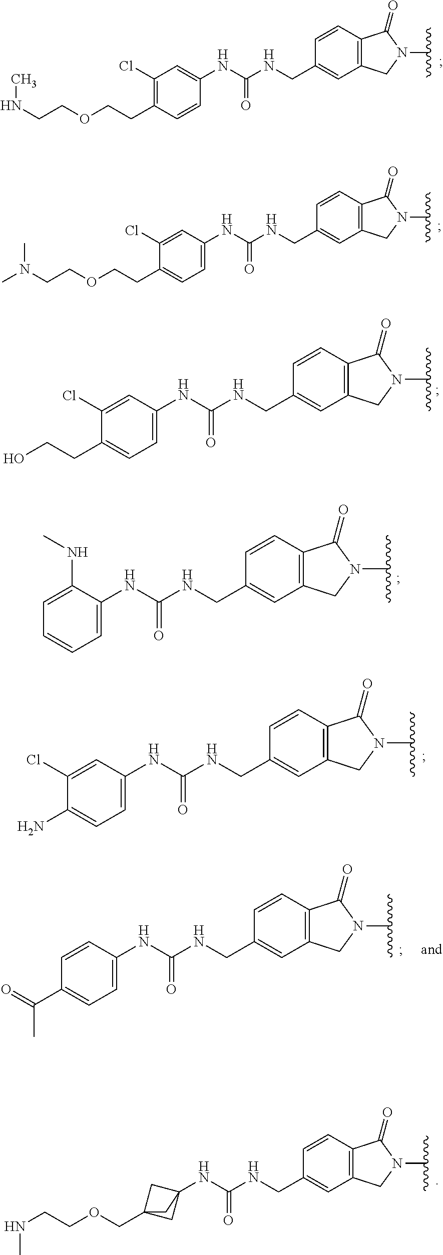

In some aspects, the targeted protein degraders comprise proteolysis-targeting chimera (PROTACs). Examples of PROTACs are known in the art (see, for example, Acta Pharmaceutica Sinica B, 2020; 10(2): 207-238.

In certain aspects, the PROTAC has the formula:

POI-L¹⁰⁰-CBN;

wherein:
POI is a compound that binds to a protein of interest;
L¹⁰⁰ is a PROTAC linker; and
CBN is a cereblon binding moiety.

Several different protein classes have been reported as PROTAC targets. In certain aspects, the protein of interest can be a nuclear hormone receptor, a translation termination factor, a transcription factor, a cyclin-dependent kinase, a tyrosine kinase, a serine/threonine kinase, or an E3 ligase.

Within the different protein classes there are several proteins of interest that can be targeted by the PROTACS described herein. In certain aspects, the protein of interest can be selected from CD33, GSPT1, BRD4, androgen receptor (AR), estrogen receptor (ER), IKZF1/3, CK1a, BCL-XL, IKZF2, IRAK4, BTK, STAT3, BTK and iMiD, BRD9, TRK, MDM2, CDK2/CDK9, CD97b, and EGFR. In some aspects, the protein of interest can be selected from BRD4, ER, and IRAK4. In some aspects, the protein of interest can be selected from BTK, BRD9, TRK, CDK2/CDK9, and STAT3.

In certain aspects, the PROTAC comprises a linker, L¹⁰⁰. PROTAC linkers have been well-studied in the art (see, for example, Troup RI, Fallan C, Baud MGJ. “Current strategies for the design of PROTAC linkers: a critical review.” Explor Target Antitumor Ther. 2020; 1:273-312. https://doi.org/10.37349/etat.2020.00018).

In certain aspects, L¹⁰⁰ can comprise an alkyl linker. In some aspects, the alkyl linker can comprise from 2 to 30 atoms. In some aspects, the alkyl linker can comprise from 5 to 25 atoms. In some aspects, the alkyl linker can comprise from 10 to 20 atoms. In some aspects, the alkyl linker can comprise 2, 3, 4, 5, 6, 7, 8. 9. 10, 11, 12, 13, 14, 15, 16, 17, 18, 19, 20, 21, 22, 23, 24, 25, 26, 27, 28, 29, or 30 atoms.

In certain aspects, L¹⁰⁰ can comprise a glycol linker. In some aspects, the glycol linker can comprise from 3 to 30 atoms. In some aspects, the glycol linker can comprise from 5 to 25 atoms. In some aspects, the alkyl linker can comprise from 10 to 20 atoms. In some aspects, the alkyl linker can comprise 3, 4, 5, 6, 7, 8. 9. 10, 11, 12, 13, 14, 15, 16, 17, 18, 19, 20, 21, 22, 23, 24, 25, 26, 27, 28, 29, or 30 atoms.

In certain aspects, L¹⁰⁰ can comprise a glycol and alkyl linker. In some aspects, the linker can comprise from 5 to 35 atoms. In some aspects, the alkyl linker can comprise from 10 to 30 atoms. In some aspects, the alkyl linker can comprise from 15 to 25 atoms. In some aspects, the alkyl linker can comprise 5, 6, 7, 8. 9. 10, 11, 12, 13, 14, 15, 16, 17, 18, 19, 20, 21, 22, 23, 24, 25, 26, 27, 28, 29, 30, 31, 32, 33, 34, or 35 atoms.

In certain aspects, L¹⁰⁰ can comprise one or more functional groups selected from polyethylene glycol (PEG), an alternative glycol groups such as a propylene glycol, alkyl, alkynyl, triazolyl, piperazinyl, piperidinyl, and mixtures thereof. It should be understood that the appropriate PROTAC linker can be selected using methods known to the skilled practitioner. In some aspects, the linker can comprise 2, 3, 4, 5, 6, 7, 8. 9. 10, 11, 12, 13, 14, 15, 16, 17, 18, 19, 20, 21, 22, 23, 24, 25, 26, 27, 28, 29, 30, 31, 32, 33, 34, or 35 atoms.

Typically, and in certain embodiments, the PROTAC can comprise a cereblon binding moiety (CNB). In some aspects, the cereblon binding moiety can be selected from:

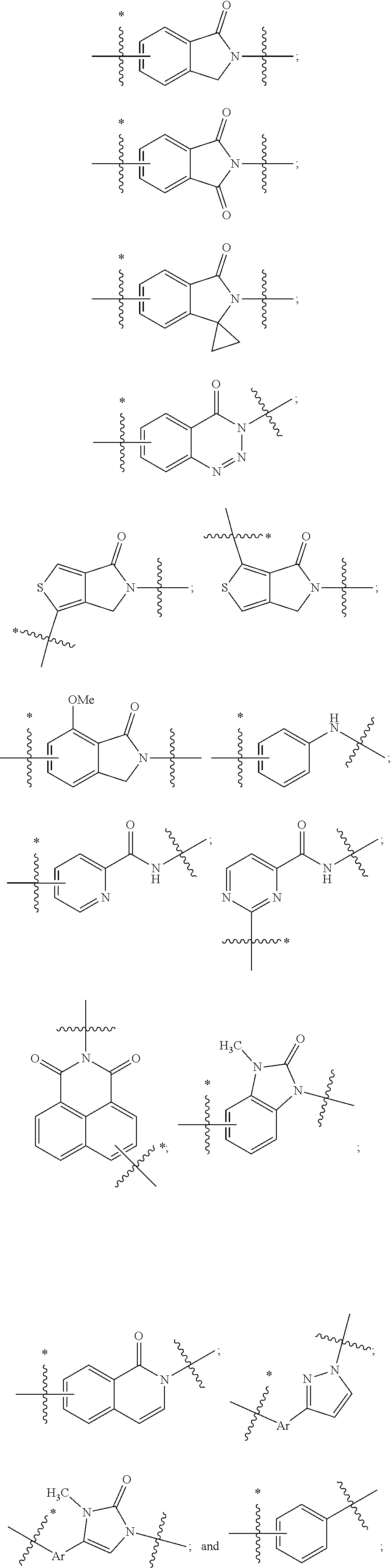

wherein
[symbol] indicates the point of attachment to A′; and
[symbol] indicates the point of attachment to L¹⁰⁰.

In some aspects, the PROTAC can be a compound having the formula:

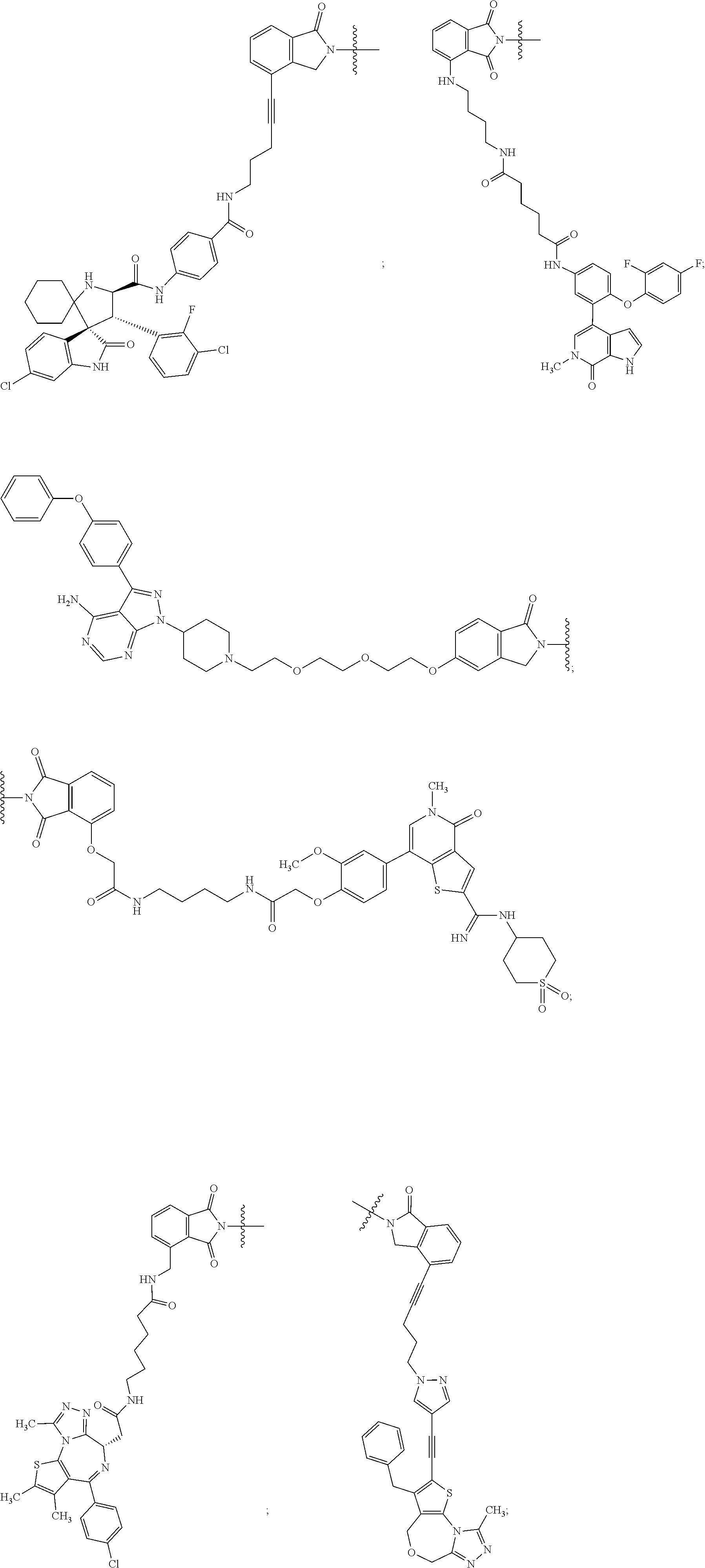

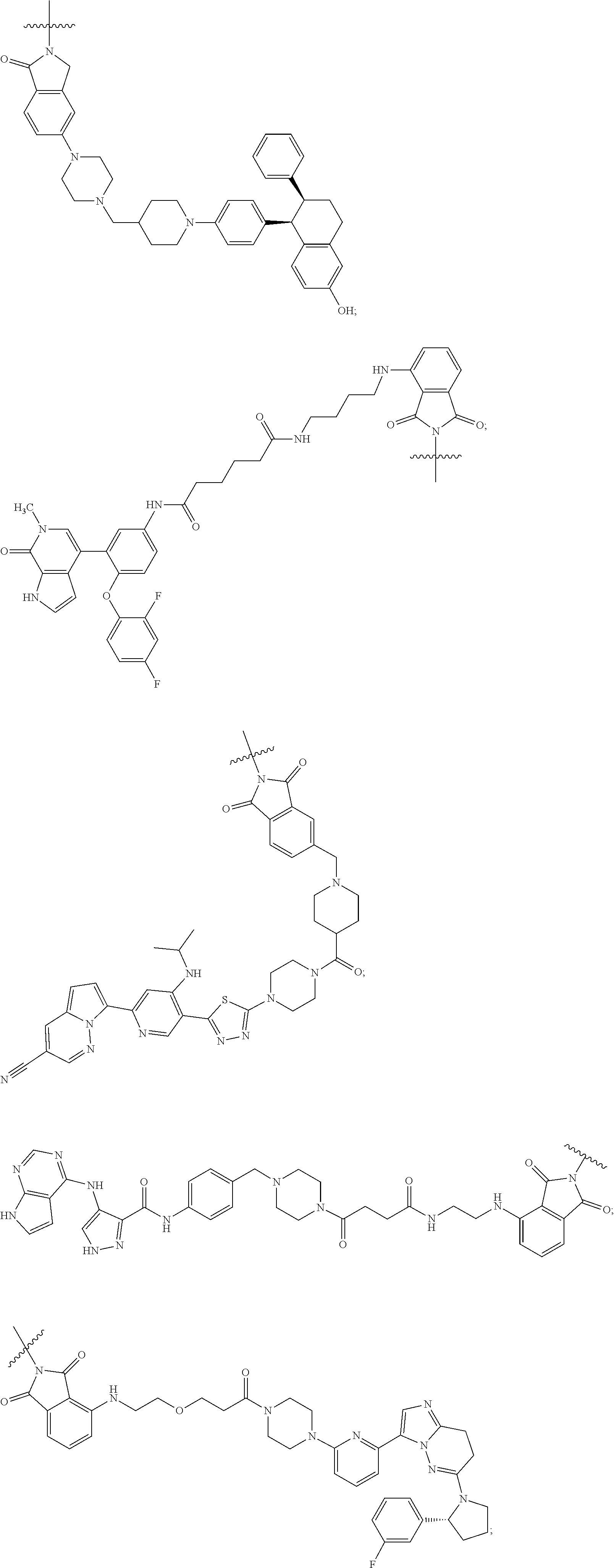

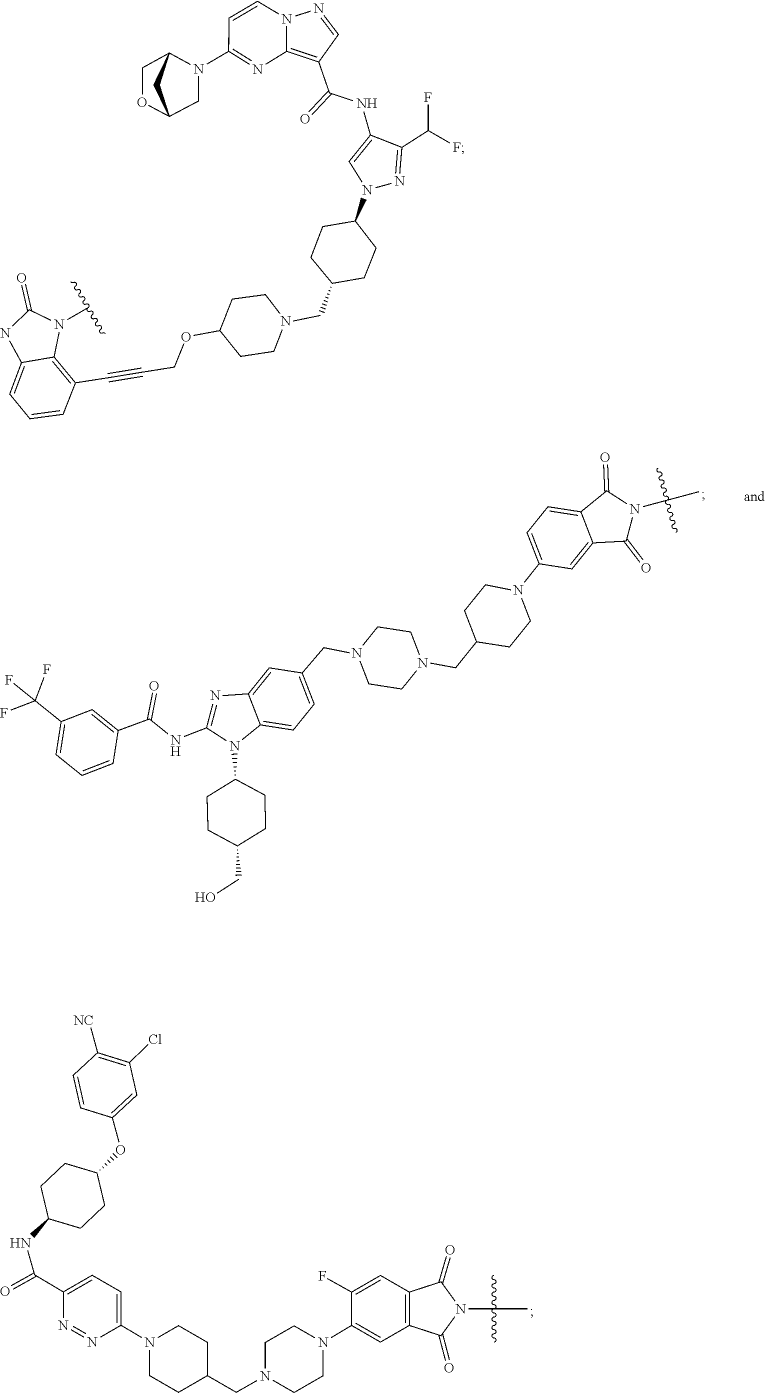

wherein [symbol] denotes the point of attachment to A′.

### III. Stability and Solubility Enhancers

The stability and/or solubility of the conjugates described herein can be improved through functionalization at R². In certain aspects, where an improvement of stability and/or solubility is not needed, R² can be hydrogen. In other aspects, where additional stability and/or solubility is desired. R² can be a group other than hydrogen.

In certain aspects, R² can be a group that imparts stability to the conjugate. In some aspects, R² is selected from C₂-C₆alkenyl, C₁-C₆alkyl; C₂-C₆alkynyl, benzyl, C₃-C₆cycloalkyl, and C₃-C₆cycloalkyl(C₁-C₃alkyl). In some aspects, R² is C₁-C₆alkyl. In some aspects, R² is methyl.

In certain aspects, R² can be a group that imparts solubility to the conjugate. In some aspects, R² can be selected from:

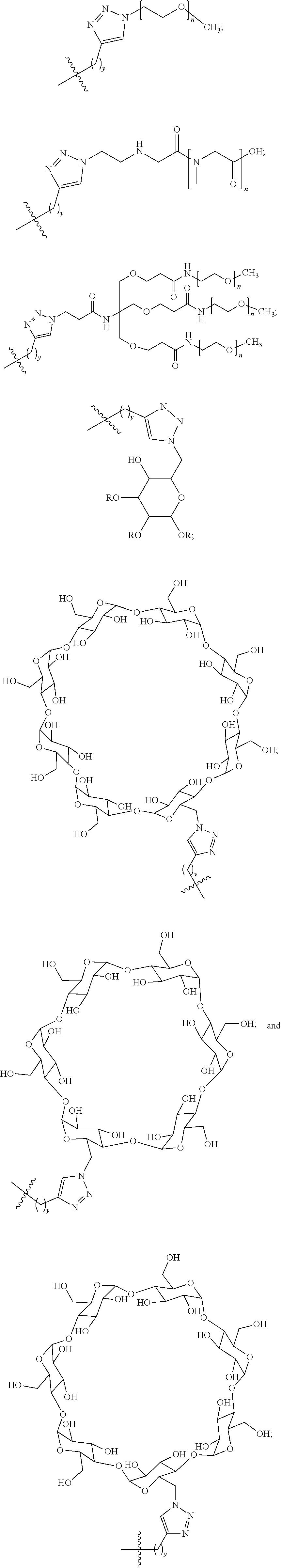

wherein:
each n is independently 1, 2, 3, 4, or 5;
each y is independently 1 or 2; and
each R is independently hydrogen, C₆H₁₁O₅, Cl₂H₂₁O₁₀, C₁₈H₃₁O₁₅, or C₂₄H₄₁O₂₀.

### IV. Conjugates

The present disclosure provides conjugates of one or more inducers of protein-protein interaction, a linker, and a binding moiety.

In some aspects, the present disclosure provides a compound of formula (I),

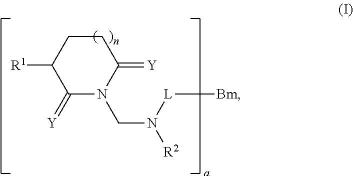

or a pharmaceutically acceptable salt thereof, wherein:
a is from 1 to 10;
n is 0 or 1;
R¹ is a compound that induces a protein-protein interaction;
R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;
each Y is independently S or 0;
L is a cleavable linker; and
Bm is a binding moiety that is capable of specifically binding to a protein. In some aspects, the protein is on a cell surface.

In some aspects, the present disclosure provides a conjugate of formula (XXXII):

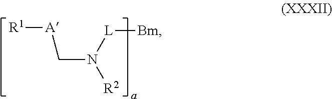

or a pharmaceutically acceptable salt thereof, wherein:
a is from 1 to 10;
A′ is

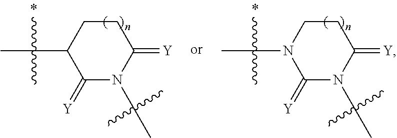

wherein
   n is 0 or 1;
   each Y is independently S or 0;
   [symbol] indicates the point of attachment to R¹; and
   [symbol] indicates the point of attachment to the methylene group;
R¹, together with A′, is a compound that induces a protein-protein interaction;
R² is selected from hydrogen, a group that provides stability to R¹-A′, a group that provides solubility to R¹-A′, and a group that provides stability and solubility to R¹-A′;
L is a cleavable linker; and
Bm is a binding moiety that is capable of specifically binding to a protein. In some aspects, the protein is on a cell surface.

In some aspects, the protein that the Bm binds to is HER2 and R¹ binds to G1 to S Phase Transition 1 (GSPT1). In some aspects, the protein that the Bm binds to is CD33 and R¹ binds to mouse double minute 2 homolog (MDM2). In some aspects, the protein that the Bm binds to is prostate specific membrane antigen (PSMA) and R¹ binds to androgen receptor (AR). In some aspects, the protein that the Bm binds to is CD33 and R¹ binds to bromodomain-containing protein 4 (BRD4). In some aspects, the protein that the Bm binds to is CD33 and R¹ binds to GSPT1. In some aspects, the protein that the Bm binds to is CD79b and R¹ binds to IRAK4. In some aspects, the protein that the Bm binds to is HER2 and R¹ binds to BRD4. In some aspects, the protein that the Bm binds to is BCMA and R¹ binds to BRD4. In some aspects, the protein that the Bm binds to is HER2 and R¹ binds to ER.

In some aspects, the conjugates described herein have in vitro anti-proliferative activity against a tumor cell line. In some aspects, the conjugates comprising one or more compounds that induce a protein-protein interaction and a binding moiety have in vitro anti-proliferative activity of at least about 50%, at least about 60%, at least about 70%, at least about 80%, at least about 90%, at least about 95%, or at least about 100% higher than the compound(s) or the binding moiety alone. In some aspects, the conjugates comprising one or more compounds that induce a protein-protein interaction and a binding moiety have an in vitro anti-proliferative activity at least about 2 fold, at least about 3 fold, at least about 4 fold, at least about 5 fold, at least about 6 fold, at least about 7 fold, at least about 8 fold, at least about 9 fold, at least about 10 fold higher than the compound(s) or the binding moiety alone.

In some aspects, the conjugates comprising one or more compounds that induce a protein-protein interaction and a binding moiety have an in vitro anti-proliferative activity against a BT-474 breast cancer cell line, e.g., higher anti-proliferative activity against a BT-474 breast cancer cell line, compared to the compound(s) alone or the binding moiety alone. In some aspects, the conjugates comprising one or more compounds that induce a protein-protein interaction and a binding moiety have an in vitro anti-proliferative activity against an SK-BR-3 breast cancer cell line, e.g., higher anti-proliferative activity against an SK-BR-3 breast cancer cell line, compared to the compound(s) alone or the binding moiety alone. In some aspects, the conjugates comprising one or more compounds that induce a protein-protein interaction and a binding moiety have an in vitro anti-proliferative activity against an NCI-N87 gastric cancer cell line, e.g., higher anti-proliferative activity against a NCI-N87 gastric cancer cell line, compared to the compound(s) alone or the binding moiety alone. In some aspects, the conjugates comprising one or more compounds that induce a protein-protein interaction and a binding moiety have an in vitro anti-proliferative activity against a Daudi lymphoma cell line, e.g., higher anti-proliferative activity against a Daudi lymphoma cell line, compared to the compound(s) alone or the binding moiety alone. In some aspects the conjugates comprising one or more compounds that induce a protein-protein interaction and a binding moiety have an in vitro anti-proliferative activity against the HL-60 acute myeloid leukemia cell line, e.g., higher anti-proliferative activity against a HL-60 acute myeloid leukemia cell line, compared to the compound(s) or the binding moiety alone. In some aspects, the conjugates comprising one or more compounds that induce a protein-protein interaction and a binding moiety have an in vitro anti-proliferative activity against a Ramos non-Hodgkins lymphoma cell line, e.g., higher anti-proliferative activity against a Ramos non-Hodgkins lymphoma cell line, compared to the compound(s) alone or the binding moiety alone. In some aspects the conjugates described herein are capable of maintaining their anti-proliferative activity in the presence of human serum. In some aspects, the conjugates described herein can be used in the treatment of cancers.

In some aspects, a conjugate provided herein can be used in the treatment of breast cancer, gastric cancer, non-small cell lung cancer, bile duct cancer, colon cancer, ovarian cancer, or neuregulin-1 (NRG1)-positive cancer, e.g., wherein the protein that the Bm binds to is HER2 and wherein R¹ binds to G1 to S Phase Transition 1 (GSPT1). In some aspects, a conjugate provided herein can be used in the treatment of acute myeloid leukemia, e.g., wherein the protein that the Bm binds to is CD33 and wherein R¹ binds to MDM2, GSPT1, or bromodomain-containing protein 4 (BRD4). In some aspects, a conjugate provided herein can be used in the treatment of prostate cancer, e.g., wherein the protein that the Bm binds to is prostate specific membrane antigen (PSMA) and wherein R¹ binds to androgen receptor (AR). In some aspects, a conjugate provided herein can be used in the treatment of a NHL, e.g., a B-cell NHL or a DLBCL, e.g., wherein the protein that the Bm binds to is CD79b and wherein R¹ binds to IRAK4.

### III.A. Linker

The compound(s) that induce a protein-protein interaction can be linked to the binding moiety through a glutarmide or dihydrouracil ring as described herein. As used herein, the term “linker” refers to any chemical moiety capable of connecting the binding moiety (Bm) to the nitrogen atom of the glutaramide or dihydrouracil ring within the compounds of formula (XX) or formula (XXX).

In certain aspects, the linker can contain a heterobifunctional group. In the present disclosure, the term “heterobifunctional group” refers to a chemical moiety that connects the linker of which it is a part to the binding moiety. Heterobifunctional groups are characterized as having different reactive groups at either end of the chemical moiety. Attachment to “Bm,” can be accomplished through chemical or enzymatic conjugation, or a combination of both. Chemical conjugation involves the controlled reaction of accessible amino acid residues on the surface of the binding moiety with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “Bm.” In enzymatic conjugation, an enzyme mediates the coupling of the linker with an accessible amino residue on the binding moiety. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Bm” to be utilized in subsequent chemical conjugation.

In some aspects, the heterobifunctional group is selected from:

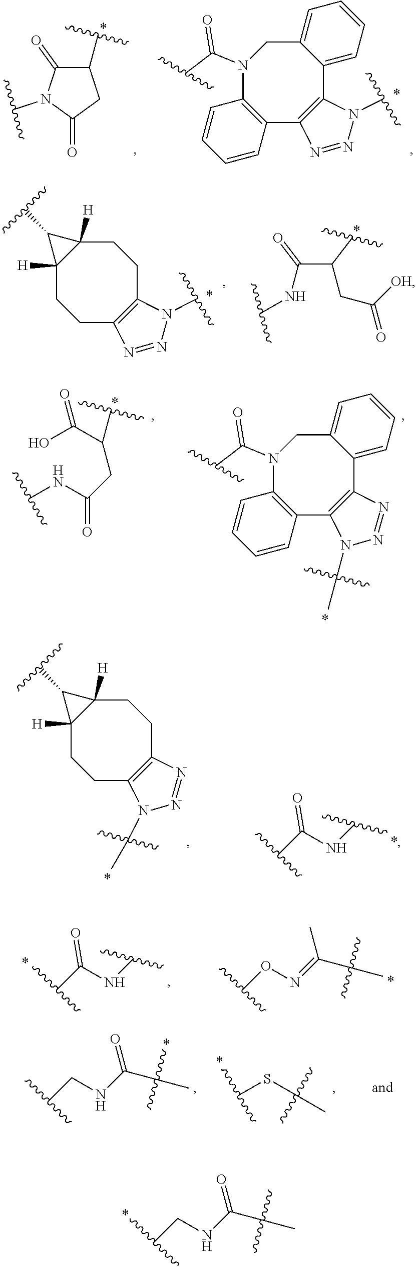

wherein
[symbol] is the point of attachment to the remaining portion of the linker; and
[symbol] the point of attachment to Bm.

In certain aspects the linker can be cleavable. In some aspects, the linker can be susceptible to acid-induced cleavage, photo-induced cleavage, bioreductive cleavage, enzymatic cleavage, or the like, at conditions under which the compound(s) that induce protein-protein interaction and/or the binding moiety can remain active.

In some aspects, the cleavable linker can be cleaved enzymatically. In some aspects, the cleavable linker can be cleaved by a protease, peptidase, esterase, β-glucuronidase, glycosidase, phosphodiesterase, phosphatase, pyrophosphatase, or lipase.

In some aspects, the cleavable linker can be cleaved by a protease. Examples of proteases include, but are not limited to, cathepsin B, VAGP tetrapeptide, and the like.

In certain aspects, the cleavable linker contains a peptide. In some aspects, the peptide is the site of cleavage of the linker, thereby facilitating release of the drug upon exposure to intracellular proteases, such as lysosomal enzymes. Peptides can be designed and optimized for enzymatic cleavage by a particular enzyme, for example, a tumor-associated protease, cathepsin B, C and D, or a plasmin protease. Examples of peptides having two amino acids include, but are not limited to, alanine-alanine (ala-ala), valine-alanine (val-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include, but are not limited to, glycine-valine-citrulline (gly-val-cit), aspartic acid-valine-citrulline (asp-val-cit), alanine-alanine-asparagine (ala-ala-asn), alanine-phenylalanine-lysine (ala-phe-lys), glycine-glycine-phenylalanine (gly-gly-phe), and glycine-glycine-glycine (gly-gly-gly). Examples of peptides having four amino acids include, but are not limited to, glycine-glycine-valine-citrulline (gly-gly-val-cit) and glycine-glycine-phenylalanine-glycine (gly-gly-phe-gly). Examples of peptides having five amino acids include, but are not limited to, glycine-glycine-valine-citrulline-glycine (gly-gly-val-cit-gly) and glycine-glycine-phenylalanine-glycine-glycine (gly-gly-phe-gly-gly). The amino acid combinations above can also be present in the reverse order (i.e., cit-val).

The peptides of the present disclosure can comprise L- or D-isomers of amino acid residues. The term “naturally-occurring amino acid” refers to Ala, Asp, Asx, Cit, Cys, Glu, Phe, Glx, Gly, His, Ile, Lys, Leu, Met, Asn, Pro, Gln, Arg, Ser, Thr, Val, Trp, and Tyr. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art.

In certain aspects, the linker (“L”) is a protease cleavable linker selected from

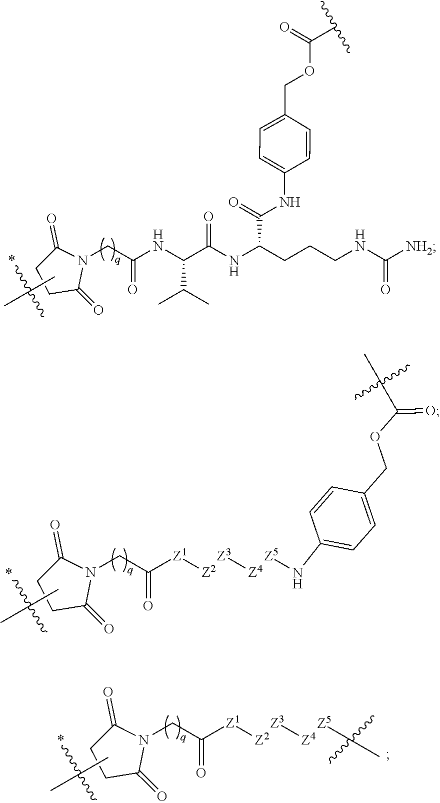

wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues;
[symbol] is the point of attachment to the parent molecular moiety; and
[symbol] is the point of attachment to the binding moiety.

In certain aspects, Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

In some aspects, Z¹ is absent or glycine; Z² is absent or selected from L-glutamine, D-glutamine, L-glutamic acid, D-glutamic acid, L-aspartic acid, D-aspartic acid, L-alanine, D-alanine, and glycine; Z³ is selected from L-valine, D-valine, L-alanine, D-alanine, L-phenylalanine, D-phenylalanine, and glycine; Z⁴ is selected from L-citrulline, D-citrulline, L-asparagine, D-asparagine, L-lysine, D-lysine, L-phenylalamine, D-phenylalanine, and glycine; and Z⁵ is absent or glycine.

In some aspects, L is

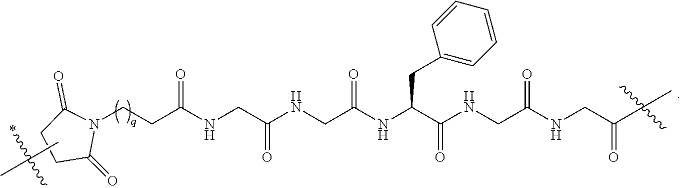

In some aspects, q is 4.

In certain aspects, L is a beta-glucuronidase cleavable linker.

In some aspects, L is a beta-glucuronidase cleavable linker which is:

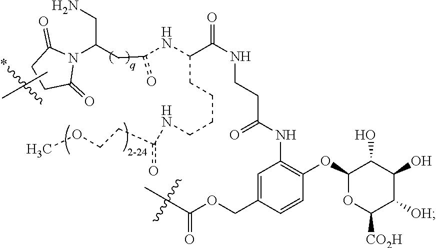

wherein:
q is from 2 to 10;
- - - is absent or a bond;
[symbol] is the point of attachment to the parent molecular moiety; and
[symbol] is the point of attachment to the binding moiety.

In some aspects, the linker is bioreducible. Bioreducible linkers take advantage of the difference in reduction potential in the intracellular compartment versus plasma. Reduced glutathione presented in tumor cells' cytoplasm is up to 1000-fold higher than that present in normal cells' cytoplasm, and the tumor cells also contain enzymes which can contribute to reduction in cellular compartments. The linkers keep conjugates intact during systemic circulation, and are selectively cleaved by the high intracellular concentration of glutathione, releasing the active drugs at the tumor sites from the non-toxic prodrugs.

In some aspects, L is a bioreducible linker selected from:

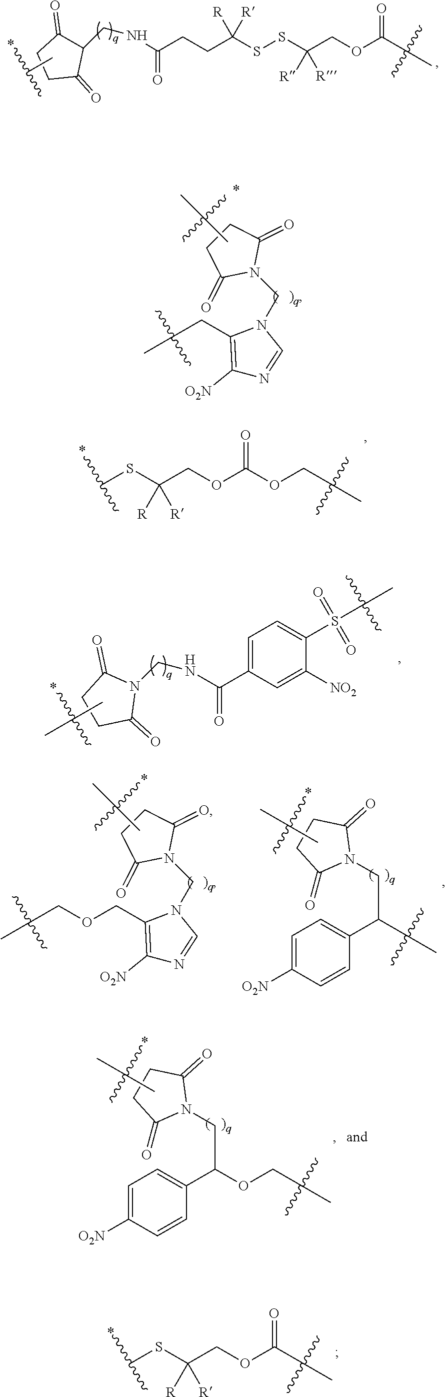

wherein:
q is from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring;
[symbol] A is the point of attachment to the parent molecular moiety; and
[symbol] is the point of attachment to the binding moiety.

In certain aspects, L is a bioreducible linker which is

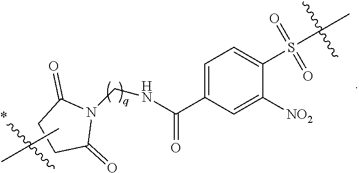

In some aspects, L is a click-to-release linker which is

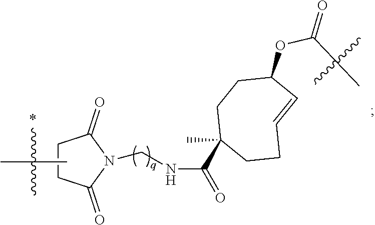

wherein:
q is from 2 to 10;
[symbol] is the point of attachment to the parent molecular moiety; and
[symbol] is the point of attachment to the binding moiety.

In some aspects, the point of attachment to the binding moiety is a cysteine, lysine, tyrosine, or glutamine in the binding moiety. In some aspects, the point of attachment to the binding moiety is a cysteine. In some aspects, the point of attachment to the binding moiety is a lysine. In some aspects, the point of attachment to the binding moiety is a tyrosine. In some aspects, the point of attachment to the binding moiety is a glutamine.

The cysteine or lysine can be an engineered (i.e., not endogenous to the binding moiety) cysteine or lysine, e.g., for site-specific conjugation. Site-specific conjugation refers to attachment through unique and defined sites on the binding moiety (e.g., antibody or antigen binding portion thereof). Site-specific conjugation is discussed, for example, in Zhou, Qun. “Site-Specific Antibody Conjugation for ADC and Beyond.” Biomedicines vol. 5.4 64. 9 Nov. 2017, doi:10.3390/biomedicines5040064, which is herein incorporated by reference in its entirety.

The cysteine or lysine, which is the point of attachment can be a cysteine or lysine that is endogenous to the binding moiety.

### III.B. BindingMoiety

The present disclosure provides one or more compounds that induces protein-protein interactions conjugated to binding moieties. The term “binding moiety,” as used herein, refers to any molecule that recognizes and binds to a cell surface marker or receptor. In certain aspects, the binding moiety binds to a protein, not limited to a polypeptide moiety. The binding moiety, in addition to targeting the compound(s) to a specific cell, tissue, or location, may also have certain therapeutic effect such as antiproliferative (cytostatic and/or cytotoxic) activity against a target cell or pathway. In certain aspects the binding moiety can comprise or can be engineered to comprise at least one chemically reactive group such as a carboxylic acid, amine, thiol, or chemically reactive amino acid moiety or side chain. In some aspects, the binding moiety can comprise a targeting moiety which binds or complexes with a cell surface molecule, such as a cell surface receptor or antigen, for a given target cell population. Following specific binding or complexing with the receptor, the cell is permissive for uptake of the targeting moiety or the conjugate, which is then internalized into the cell.

In some aspects, group “Bm” can be a peptide or a protein that binds to a cell surface receptor or antigen.

In certain aspects, group “Bm” can be an antibody, antibody fragment, or an antigen-binding fragment. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. The term “antibody” herein is used in the broadest sense and specifically covers monoclonal antibodies, single domain antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

Monoclonal antibodies that can be conjugated to the compound(s) are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture. These include, but are not limited to, the hybridoma technique, the human B cell hybridoma technique, and the EBV-hybridoma technique. Such antibodies may be of any immunoglobulin class including IgG, IgM, IgE, IgA, and IgD and any subclass thereof. The hybridoma producing the mAbs of use in this disclosure may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art.

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually performed using affinity chromatography steps, is rather cumbersome, and the product yields are low.

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, CH₂, and CH₃ regions. The first heavy-chain constant region (CH1) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in aspects when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. Using such techniques, bispecific antibodies can be prepared for conjugation to the compound inducing a protein-protein interaction in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof.

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary aspect the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other useful antibodies include fragments of antibodies such as, but not limited to, F(ab′)₂ fragments, which contain the variable region, the light chain constant region and the CH1 domain of the heavy chain can be produced by pepsin digestion of the antibody molecule, and Fab fragments, which can be generated by reducing the disulfide bridges of the F(ab′)₂ fragments. Other useful antibodies are heavy chain and light chain dimers of antibodies, or any minimal fragment thereof such as Fvs or single chain antibodies (SCAs), or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). Other human antibodies can be obtained commercially from, for example, Abgenix, Inc. (Freemont, Calif.) and Genpharm (San Jose, Calif).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

The antibody can be a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies in the conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially, for example, from Genentech (San Francisco, Calif.) or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody of the conjugates can be a monoclonal antibody, e.g. a murine monoclonal antibody, a chimeric antibody, or a humanized antibody. In some aspects, the antibody can be an antibody fragment, e.g. a Fab fragment.

Known antibodies for the treatment or prevention of cancer can be conjugated to the compound(s) described herein. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN® (rituximab; Genentech) which is a chimeric anti-CD20 monoclonal antibody for the treatment of patients with non-Hodgkin's lymphoma; OVAREX® (oregovomab; AltaRex Corporation, MA) which is a murine antibody for the treatment of ovarian cancer; Panorex (edrecolomab, Glaxo Wellcome, NC) which is a murine IgG2a antibody for the treatment of colorectal cancer; Cetuximab Erbitux (cetuximab, Imclone Systems Inc., NY) which is an anti-EGFR IgG chimeric antibody for the treatment of epidermal growth factor positive cancers, such as head and neck cancer; Vitaxin (etaracizumab, MedImmune, Inc., MD) which is a humanized antibody for the treatment of sarcoma; Campath I/H (alemtuzumab, Leukosite, MA) which is a humanized IgG1 antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (epratuzumab, Immunomedics, Inc., NJ) which is a humanized anti-CD22 IgG antibody for the treatment of non-Hodgkin's lymphoma; Smart ID10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA) which is a humanized anti-CD2 mAb for the treatment of Hodgkin's Disease or non-Hodgkin's lymphoma; Avastin (bevacizumab, Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin's lymphoma; and CEAcide (Immunomedics, NJ) which is a humanized anti-CEA antibody for the treatment of colorectal cancer.

Other antibodies useful in the conjugates include, but are not limited to, trastuzumab, gemtuzumab, pertuzumab, obinutuzumab, ofatumumab, daratumumab, STI-6129, lintuzumab, huMy9-6, belantamab, indatuximab, dinutuximab, anti-CD38 A2 antibody, buAT15/3 H3s antibody, ibritumomab, tositumomab, panitumumab, tremelimumab, ticilimumab, catumaxomab, and veltuzumab. In certain aspects, the antibody is selected from the group consisting of rituximab, trastuzumab, pertuzumab, huMy9-6, lintuzumab, and gemtuzumab. Other antibodies useful in the conjugates include, but are not limited to, polatuzumab, J591, lorvotuzumab and sacituzumab.

Other antibodies useful for the conjugates include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp100 (melanoma), MART1 (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, P A, et al Cancer Research (1997) 57, 100-105), mAbs against the CD40 antigen, such as S2C₆ mAb (Francisco, J. A., et al Cancer Res. (2000) 60:3225-3231), mAbs against the CD70 antigen, such as 1F6 mAb, and mAbs against the CD30 antigen, such as AC10. Many other internalizing antibodies that bind to tumor associated antigens can be used and have been reviewed.

Other antigens that the present conjugates can bind to include, but are not limited to, 5T4, ACE, ADRB3, AKAP-4, ALK, AOC3, APP, Axin1, AXL, B7H3, B7-H4, BCL2, BCMA, bcr-ab1, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, c113, c-MET, Cripto protein, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-11Ra, IL-1 receptor, IL-12 receptor, IL-23 receptor, IL-13 receptor, IL-22 receptor, IL-4 receptor, IL-5 receptor, IL-6, interferon receptor, integrins (including α4, αvβ, αvβ5, αvβ6, α1β4, α4β1, α4β7, α5β1, α6β4, αIIbβ3 intergins), Integrin alphaV, intestinal carboxyl esterase, KIT, LAGE-la, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PD-1, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRP, TEM1/CD248, TEM7R, tenascin C, TF, TGF-1, TGF-β2, TNF-α, TGS5, Tie 2, TIM-1, Tn Ag, TRAC, TRAIL-R¹, TRAIL-R2, TROP-2, TRP-2, TRPV1, TSHR, tumor antigen CTAA16.88, tyrosinase, UPK2, VEGF, VEGFR1, VEGFR2, vimentin, WT1, and/or XAGE1.

Antibodies that bind to antigens associated with antigen presenting cells such as CD40, OX40L, Endoglin, DEC-205, 4-1BBL, CD36, CD36, CD204, MARCO, DC-SIGN, CLEC9A, CLEC5A, Dectin 2, CLECIOA, CD206, CD64, CD32A, CD1A, HVEM, CD32B, PD-L1, BDCA-2, XCR-1, and CCR2 can also be conjugated to the compound(s) inducing protein-protein interaction.

Antibodies of a conjugate described herein can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD 19, CD22, CD28, CD79, CD90, CD 152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD 11c, CD18, CD29, CD41, CD49a, CD49b, CD49c, CD49d, CD49e, CD49f, CD 103, and CD 104. Non-limiting examples of suitable lectins are C-type, S-type, and I-type lectin.

In some aspects, the antibodies that are useful for the present disclosure include, but are not limited to, 3F8, 8H9, abagovomab, abciximab (REOPRO®), abituzumab, abrezekimab, abrilumab, actoxumab, adalimumab (HUMIRA®), adecatumumab, aducanumab, afasevikumab, afelimomab, afutuzumab, alacizumab, ALD518, alemtuzumab (CAMPATH®), alirocumab (PRALUENT®), altumomab, amatuximab, anatumomab, andecaliximab, anetumab, anifrolumab, anrukinzumab, apolizumab, aprutumab, arcitumomab (CEA-SCAN®), ascrinvacumab, aselizumab, atidortoxumab, atlizumab (tocilizumab, ACTEMRA®, ROACTEMRA®), atezolizumab (TECENTRIQ®), atinumab, atorolimumab, avelumab (Bavencio), azintuxizumab, belantamab, bapineuzumab, basiliximab (SIMULECT®), bavituximab, BCD-100, bectumomab (LYMPHOSCAN®), begelomab, belantamab, belimumab (BENLYSTA®), bemarituzumab, benralizumab (FASENRA®), bermekimab, bersanlimab, bertilimumab, besilesomab (SCINITIMUN®), bevacizumab (AVASTIN®), bezlotoxumab (ZINPLAVA®), biciromab (FIBRISCINT®), bimagrumab, bimekizumab, birtamimab, bivatuzumab, bleselumab, blinatumomab, blontuvetmab, blosozumab, bococizumab, brazikumab, brentuximab, briakinumab, brodalumab (SILIQ™), brolucizumab (BEOVU® brontictuzumab, burosumab (CRYSVITA®), cabiralizumab, caplacizumab (CABLIVI®), camidanlumab, camrelizumab, canakinumab (ILARIS®), cantuzumab, capromab, carlumab, carotuximab, catumaxomab (REMOVAB®), cBR96, CC49, cedelizumab, cemiplimab (LIBTAYO®), cergutuzumab, certrelimab, certolizumab, cetuximab (ERBITUX®), cibisatamab, cirmtuzumab, citatuzumab, cixutumumab, clazakizumab, clenoliximab, clivatuzumab, codrituzumab, cofetuzumab, coltuximab, conatumumab, concizumab, cosfroviximab, CR6261, crenezumab, crizanlizumab (ADAKVEO®), crotedumab, cusatuzumab, dacetuzumab, daclizumab (ZINBRYTA®), dalotuzumab, dapirolizumab, daratumumab (DARZALEX®), dectrekumab, demcizumab, denintuzumab, denosumab (PROLIA®), depatuxizumab, derlotuximab, detumomab, dezamizumab, dinutuximab (UNITUXIN®), diridavumab, domagrozumab, dostarlimab, dorlimomab, dorlixizumab, drozitumab, DS-8201, duligotuzumab, dupilumab (DUPIXENT®), durvalumab (IMFINZI®), dusigitumab, ecromeximab, eculizumab (SOLIRIS®), edobacomab, edrecolomab (PANOREX®), efalizumab (RAPTIVA®), efungumab (MYCOGRAB®), eldelumab, elezanumab, elgemtumab, elotuzumab (EMPLICITI®), elsilimomab, emactuzumab emapalumab (GAMIFANT®), emibetuzumab, emicizumab (HEMLIBRA®), enapotamab, enavatuzumab, enfortumab (PADCEV®), enlimomab, enoblituzumab, enokizumab, enoticumab, ensituximab, epitumomab, eptinezumab (VYEPTI®epratuzumab, erenumab (AIMOVIG®), erlizumab, ertumaxomab (REXOMUN®), etaracizumab (ABEGRIN®), etigilimab, etrolizumab, evinacumab, evolocumab (REPATHA®), exbivirumab, fanolesomab (NEUTROSPEC®), faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab (HUZAF®), foralumab, foravirumab, fremanezumab (AJOVY®), fresolimumab, frovocimab, frunevetmab, fulranumab, futuximab, galcanezumab (EMGALITY®), galiximab, gancotamab, ganitumab, gantenerumab, gavilimomab, gedivumab, gemtuzumab, gevokizumab, gilvetmab, gimsilumab, girentuximab, glembatumumab, golimumab (SIMPONI®), gomiliximab, guselkumab (TREMFYA®), huMy9-6, ianalumab, ibalizumab (TROGARZO®), IBI308, ibritumomab, icrucumab, idarucizumab (PRAXBIND®), ifabotuzumab, igovomab (INDIMACIS-125), iladatuzumab, IMAB362, imalumab, imaprelimab, imeiromab (MYOSCINT®), imgatuzumab, inclacumab, indatuximab, indusatumab, inebilizumab, infliximab (REMICADE®), intetumumab, inolimomab, inotuzumab, iomab-B, ipilimumab, iratumumab, isatuximab (SARCLISA®), iscalimab, istiratumab, itolizumab, ixekizumab (TALTZ®), keliximab, labetuzumab (CEA-CIDE™), lacnotuzumab, ladiratuzumab, lampalizumab, lanadelumab (TAKHZYRO®), landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, lilotomab, lintuzumab, lirilumab, lodelcizumab, lokivetmab, loncastuximab, lorvotuzumab, losatuxizumab, lucatumumab, lulizumab, lumiliximab, lumretuzumab, lupartumab, lutikizumab, mapatumumab, margetuximab, marstacimab, maslimomab, matuzumab, mavrilimumab, mepolizumab (NUCALA®), metelimumab, milatuzumab, minretumomab, mirikizumab, mirvetuximab, mitumomab, modotuximab, molalizumab, mogamulizumab (POTELIGEO®), morolimumab, mosunetuzumab, motavizumab (NUMAX®), moxetumomab (LUMOXITI®), muromonab-CD3 (ORTHOCLONE OKT3®), nacolomab, namilumab, naptumomab, naratuximab, narnatumab, natalizumab (TYSABRI®), navicixizumab, navivumab, naxitamab, nebacumab, necitumumab (PORTRAZZA®), nemolizumab, NEOD001, nerelimomab, nesvacumab, netakimab, nimotuzumab (THERACIM®), nirsevimab, nivolumab, nofetumomab, obiltoxaximab (ANTHIM®), obinutuzumab, ocaratuzumab, ocrelizumab (OCREVUS®), odulimomab, ofatumumab (ARZERRA®), olaratumab (LARTRUVO®), oleclumab, olendalizumab, olokizumab, omalizumab (XOLAIR®), omburtamab, OMS721, onartuzumab, ontecizumab, ontuxizumab, onvatilimab, opicinumab, oportuzumab, oregovomab (OVAREX), orticumab, otelixizumab, otilimab, otlertuzumab, oxelumab, ozanezumab, ozogamicin, ozoralizumab, pagibaximab, palivizumab (SYNAGIS®), pamrevlumab, panitumumab (VECTIBIX®), pankomab, panobacumab, parsatuzumab, pascolizumab, pasotuxizumab, pateclizumab, patritumab, PDR001, pembrolizumab, pemtumomab (THERAGYN®), perakizumab, pertuzumab (OMNITARG®), pexelizumab, pidilizumab, pinatuzumab, pintumomab, placulumab, polatuzumab (Polivy), prezalumab, plozalizumab, pogalizumab, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab (LUCENTIS®), ravagalimab, ravulizumab (ULTOMIRIS®), raxibacumab, refanezumab, regavirumab, REGN-EB3, renatlimab, remtolumab, reslizumab (CINQAIR®), rilotumumab, rinucumab, risankizumab (SKYRIZI®), rituximab (RITUXAN®), rivabazumab, rmab, robatumumab, roledumab, romilkimab, romosozumab (EVENITY®), rontalizumab, rosmantuzumab, rovalpituzumab, rovelizumab (LEUKARREST®), rozanolixizumab, ruplizumab (ANTOVA), SA237, sacituzumab, samalizumab, samrotamab, sarilumab (KEVZARA®), satralizumab, satumomab pendetide, secukinumab (COSENTYX®), selicrelumab, seribantumab, setoxaximab, setrusumab, sevirumab, SGN-CD19A, SHP647, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, STI-6129, sulesomab (LEUKOSCAN®), suptavumab, sutimlimab, suvizumab, suvratoxumab, tabalumab, tacatuzumab (AFP-CIDE®), tadocizumab, talacotuzumab, talizumab, tamtuvetmab, tanezumab, taplitumomab paptox, tarextumab, tavolimab, tefibazumab (AUREXIS®), telimomab, telisotuzumab, tesidolumab, tetraxetan, tetulomab, tenatumomab, teneliximab, teprotumumab (TEPEZZA®), teplizumab, tezepelumab, TGN1412, tibulizumab, ticilimumab (TREMELIMUMAB®), tigatuzumab, timigutuzumab, timolumab, tiragolumab, tiragotumab, tislelizumab, tisotumab, tiuxetan, tildrakizumab (ILUMYA®), TNX-650, tocilizumab (atlizumab, ACTEMRA®), tomuzotuximab, toralizumab, tosatoxumab, tositumomab (BEXXAR®), tovetumab, tralokinumab, trastuzumab (HERCEPTIN®), TRBS07, tregalizumab, tremelimumab, trevogrumab, tucotuzumab, tuvirumab, urtoxazumab, ustekinumab (STELERA®), ublituximab, ulocuplumab, urelumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab (NUVION®), vobarilizumab, volociximab (HUMASPECT®), vonlerolizumab, vopratelimab, vorsetuzumab, votumumab, vunakizumab, xentuzumab, XMAB-5574, zalutumumab (HuMEX-EGFr), zanolimumab (HuMAX-CD4), zatuximab, zenocutuzumab, ziralimumab, zolbetuximab or zolimomab. In some aspects, the antibodies that are useful for the present disclosure include, but are not limited to J591 and belantamab.

In some aspects, a binding moiety is an antibody or antigen binding portion thereof that comprises the 6 CDRs of an antibody in Table A (i.e., the 3 CDRs of the variable heavy chain or heavy chain and the 3 CDRs of the variable light chain or light chain of the same antibody).

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, a binding moiety is an antibody or antigen binding portion thereof that comprises the 6 Kabat-defined CDRs of an antibody in Table A (i.e., the 3 Kabat-defined CDRs of the variable heavy chain or heavy chain and the 3 Kabat-defined CDRs of the variable light chain or light chain of the same antibody).

The CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, a binding moiety is an antibody or antigen binding portion thereof that comprises the 6 Chothia-defined CDRs of an antibody in Table A (i.e., the 3 Chothia-defined CDRs of the variable heavy chain or heavy chain and the 3 Chothia-defined CDRs of the variable light chain or light chain of the same antibody). In some aspects, a binding moiety is an antibody or antigen binding portion thereof that comprises one or more CDRs, in which the Chothia and Kabat CDRs have the same amino acid sequence. In some aspects, a binding moiety is an antibody or antigen binding portion thereof that comprises comprises a combinations of Kabat CDRs and Chothia CDRs of an antibody in Table A.

In some aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, a binding moiety is an antibody or antigen binding portion thereof that comprises the 6 IMGT-defined CDRs of an antibody in Table A (i.e., the 3 IMGT-defined CDRs of the variable heavy chain or heavy chain and the 3 IMGT-defined CDRs of the variable light chain or light chain of the same antibody), for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, a binding moiety is an antibody or antigen binding portion thereof that comprises the 6 MacCallum-defined CDRs of an antibody in Table A (i.e., the 3 MacCallum-defined CDRs of the variable heavy chain or heavy chain and the 3 MacCallum-defined CDRs of the variable light chain or light chain of the same antibody), for example as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, a binding moiety is an antibody or antigen binding portion thereof that comprises the 6 AbM-defined CDRs of an antibody in Table A (i.e., the 3 AbM-defined CDRs of the variable heavy chain or heavy chain and the 3 AbM-defined CDRs of the variable light chain or light chain of the same antibody) as determined by the AbM numbering scheme.

In some aspects, a binding moiety is an antibody or antigen binding portion thereof that binds to CD33. In some aspects, an antibody or antigen binding portion thereof binds to CD33 and comprises the 6 CDRs of an anti-CD33 antibody disclosed in U.S. Pat. No. 10,711,062, which is herein incorporated by reference in its entirety. In some aspects, an antibody or antigen binding portion thereof binds to CD33 and comprises the VH and VL of an anti-CD33 antibody disclosed in U.S. Pat. No. 10,711,062. In some aspects, an antibody or antigen binding portion thereof binds to CD33 and comprises the 6 CDRs of an anti-CD33 antibody disclosed in U.S. Patent Application Publication No. 2021/0047404, which is herein incorporated by reference in its entirety. In some aspects, an antibody or antigen binding portion thereof binds to CD33 and comprises the VH and VL of an anti-CD33 antibody disclosed in U.S. Patent Application Publication No. 2021/0047404. In some aspects, an antibody or antigen binding portion thereof binds to CD33 and comprises the 6 CDRs of an anti-CD33 antibody disclosed in U.S. Patent Application Publication No. 2020/0297764, which is herein incorporated by reference in its entirety. In some aspects, an antibody or antigen binding portion thereof binds to CD33 and comprises the VH and VL of an anti-CD33 antibody disclosed in U.S. Patent Application Publication No. 2020/0297764. In some aspects, an antibody or antigen binding portion thereof binds to CD33 and comprises the 6 CDRs of an anti-CD33 antibody provided in Table A (e.g., CD33-A, CD33-B, or CD33-C). In some aspects, an antibody or antigen binding portion thereof binds to CD33 and comprises the 6 CDRs of anti-CD33 antibody CD33-D provided in Table A. In some aspects, an antibody or antigen binding portion thereof binds to CD33 and comprises the 6 CDRs of anti-CD33 antibody CD33 huMy9-6 provided in Table A. In some aspects, an antibody or antigen binding portion thereof binds to CD33 and comprises a VH comprising the amino acid sequence of SEQ ID NO:1 and a VL comprising the amino acid sequence of SEQ ID NO:2. In some aspects, an antibody or antigen binding portion thereof binds to CD33 and comprises a VH comprising the amino acid sequence of SEQ ID NO:3 and a VL comprising the amino acid sequence of SEQ ID NO:4. In some aspects, an antibody or antigen binding portion thereof binds to CD33 and comprises a VH comprising the amino acid sequence of SEQ ID NO:5 and a VL comprising the amino acid sequence of SEQ ID NO:6. In some aspects, an antibody or antigen binding portion thereof binds to CD33 and comprises a VH comprising the amino acid sequence of SEQ ID NO:27 and a VL comprising the amino acid sequence of SEQ ID NO:28. In some aspects, an antibody or antigen binding portion thereof binds to CD33 and comprises a VH comprising the amino acid sequence of SEQ ID NO:22 and a VL comprising the amino acid sequence of SEQ ID NO:23. In some aspects, an antibody or antigen binding portion thereof binds to CD33 and comprises a heavy chain comprising the amino acid sequence of SEQ ID NO:24 and a light chain comprising the amino acid sequence of SEQ ID NO:25.

In some aspects, a binding moiety is an antibody or antigen binding portion thereof that binds to PSMA. In some aspects, an antibody or antigen binding portion thereof binds to PSMA and comprises the 6 CDRs of an anti-PSMA antibody disclosed in U.S. Patent Application Publication No. 2019/0022205, which is herein incorporated by reference in its entirety. In some aspects, an antibody or antigen binding portion thereof binds to PSMA and comprises the VH and VL of an anti-PSMA antibody disclosed in U.S. Patent Application Publication No. 2019/0022205. In some aspects, an antibody or antigen binding portion thereof binds to PSMA and comprises the 6 CDRs of an anti-PSMA antibody disclosed in U.S. Pat. No. 10,100,126, which is herein incorporated by reference in its entirety. In some aspects, an antibody or antigen binding portion thereof binds to PSMA and comprises the VH and VL of an anti-PSMA antibody disclosed in U.S. Pat. No. 10,100,126. In some aspects, an antibody or antigen binding portion thereof binds to PSMA and comprises the 6 CDRs of an anti-PSMA antibody disclosed in U.S. Pat. No. 8,470,330, which is herein incorporated by reference in its entirety. In some aspects, an antibody or antigen binding portion thereof binds to PSMA and comprises the VH and VL of an anti-PSMA antibody disclosed in U.S. Pat. No. 8,470,330. In some aspects, an antibody or antigen binding portion thereof binds to PSMA and comprises the 6 CDRs of an anti-PSMA antibody provided in Table A (i.e., PSMA-A, PSMA-B, or PSMA-C). In some aspects, an antibody or antigen binding portion thereof binds to PSMA and comprises the 6 CDRs of anti-PSMA antibody PSMA-D provided in Table A. In some aspects, an antibody or antigen binding portion thereof binds to PSMA and comprises a VH comprising the amino acid sequence of SEQ ID NO:7 and a VL comprising the amino acid sequence of SEQ ID NO:8. In some aspects, an antibody or antigen binding portion thereof binds to PSMA and comprises a VH comprising the amino acid sequence of SEQ ID NO:9 and a VL comprising the amino acid sequence of SEQ ID NO:10. In some aspects, an antibody or antigen binding portion thereof binds to PSMA and comprises a VH comprising the amino acid sequence of SEQ ID NO:11 and a VL comprising the amino acid sequence of SEQ ID NO:12. In some aspects, an antibody or antigen binding portion thereof binds to PSMA and comprises a VH comprising the amino acid sequence of SEQ ID NO:29 and a VL comprising the amino acid sequence of SEQ ID NO:30.

In some aspects, a binding moiety is an antibody or antigen binding portion thereof that binds to HER2. In some aspects, an antibody or antigen binding portion thereof binds to HER2 and comprises the 6 CDRs of an anti-HER2 antibody disclosed in U.S. Pat. No. 7,862,817, which is herein incorporated by reference in its entirety. In some aspects, an antibody or antigen binding portion thereof binds to HER2 and comprises the VH and VL of an anti-HER2 antibody disclosed in U.S. Pat. No. 7,862,817. In some aspects, an antibody or antigen binding portion thereof binds to HER2 and comprises the 6 CDRs of an anti-HER2 antibody disclosed in U.S. Pat. No. 7,850,966, which is herein incorporated by reference in its entirety. In some aspects, an antibody or antigen binding portion thereof binds to HER2 and comprises the VH and VL of an anti-HER2 antibody disclosed in U.S. Pat. No. 7,850,966. In some aspects, an antibody or antigen binding portion thereof binds to HER2 and comprises the 6 CDRs of an anti-HER2 antibody disclosed in PCT International Publication No. WO2016/201051, which is herein incorporated by reference in its entirety. In some aspects, an antibody or antigen binding portion thereof binds to HER2 and comprises the VH and VL of an anti-HER2 antibody disclosed in PCT International Publication No. WO2016/201051. In some aspects, an antibody or antigen binding portion thereof binds to HER2 and comprises the 6 CDRs of an anti-HER2 antibody provided in Table A (i.e., HER2-A, HER2-B, or HER2-C). In some aspects, an antibody or antigen binding portion thereof binds to HER2 and comprises a VH comprising the amino acid sequence of SEQ ID NO:13 and a VL comprising the amino acid sequence of SEQ ID NO:14. In some aspects, an antibody or antigen binding portion thereof binds to HER2 and comprises a VH comprising the amino acid sequence of SEQ ID NO:15 and a VL comprising the amino acid sequence of SEQ ID NO:16. In some aspects, an antibody or antigen binding portion thereof binds to HER2 and comprises a VH comprising the amino acid sequence of SEQ ID NO:17 and a VL comprising the amino acid sequence of SEQ ID NO:18.

In some aspects, a binding moiety is an antibody or antigen binding portion thereof that binds to CD20. In some aspects, an antibody or antigen binding portion thereof binds to CD20 and comprises the 6 CDRs of anti-CD20 antibody CD20-A provided in Table A. In some aspects, an antibody or antigen binding portion thereof binds to CD20 and comprises a VH comprising the amino acid sequence of SEQ ID NO:31 and a VL comprising the amino acid sequence of SEQ TD NO:32.

In some aspects, a binding moiety is an antibody or antigen binding portion thereof that binds to CD79b. In some aspects, an antibody or antigen binding portion thereof binds to CD79b and comprises the 6 CDRs of anti-CD79b antibody CD79b-A provided in Table A. In some aspects, an antibody or antigen binding portion thereof binds to CD79b and comprises a VH comprising the amino acid sequence of SEQ TD NO:33 and a VL comprising the amino acid sequence of SEQ ID NO:34.

In some aspects, a binding moiety is an antibody or antigen binding portion thereof that binds to BCMA. In some aspects, an antibody or antigen binding portion thereof binds to BCMA and comprises the 6 CDRs of anti-BCMA antibody BCMA-A provided in Table A. In some aspects, an antibody or antigen binding portion thereof binds to BCMA and comprises the amino acid sequences of SEQ TD NO:35 and SEQ ID NO:36.

TABLE A
Exemplary Antibody or Antigen Binding Portion Thereof Sequences
(CDR sequences shown in bold and underlined)

| | |
|---|---|
| CD33- | QVQLVQSGAEVKKPGASVKISCKASGYTFTDYNLHWVRQAPGQGL |
| A VH | EWIGFIYPSNRITGYAQKFQGRATLTVDNSTSTAYMELSSLRSEDTA |
| | VYYCARSDVDYFDYWGQGTLLTVSS (SEQ ID NO: 1) |
| CD33- | DIVLTQSPDSLAVSLGERATINCRASQSVSTSTYSYMHWYQQKPGQ |
| A VL | PPKLLIKYASNLESGVPDRFSGSGSGTDFTLTISSLQAEDVAVYYCQH |
| | SWEIPLTFGQGTKLEIK (SEQ ID NO: 2) |
| CD33- | QVQLVQSGAEVKKPGASVKVSCKASGYTFTNYDINWVRQAPGQGL |
| B VH | EWIGWIYPGDGSTKYNEKFKAKATLTADTSTSTAYMELRSLRSDDT |
| | AVYYCASGYEDAMDYWGQGTTVTVSS (SEQ ID NO: 3) |
| CD33- | DIQMTQSPSSLSASVGDRVTINCKASQDINSYLSWFQQKPGKAPKTL |
| B VL | IYRANRLVDGVPSRFSGSGSGQDYTLTISSLQPEDFATYYCLQYDEF |
| | PLTFGGGTKVEIK (SEQ ID NO: 4) |
| CD33- | EVQLQQSGPEVVKPGASVKISCKASGYTFTDYNMHWVKQAHGQSL |
| C VH | EWIGYIYPYNGGTGYNQKFKSKATLTVDNSASTAYMEVRSLTSEDT |
| | AVYYCARGRPAMDYWGQGTLVTVSS (SEQ ID NO: 5) |
| CD33- | EIVLTQSPATLSVSLGQRATISCRASESVDNYGISFMNWFQQKPGQP |
| C VL | PKLLIYAASNQGSGVPARFSGSGSGTDFTLTIHPMEEDDTAMYFCQQ |
| | SKEVPWTFGGGTKLEIK (SEQ ID NO: 6) |
| CD33- | EVQLVQSGAEVKKPGSSVKVSCKASGYTITDSNIHWVRQAPGQSLE |
| D VH | WIGYIYPYNGGTDYNQKFKNRATLTVDNPTNTAYMELSSLRSEDT |
| | AFYYCVNGNPWLAYWGQGTLVTVSS (SEQ ID NO: 27) |
| CD33- | DIQLTQSPSTLSASVGDRVTITCRASESLDNYGIRFLTWFQQKPGKA |
| D VL | PKLLMYAASNQGSGVPSRFSGSGSGTEFTLTISSLQPDDFATYYCQQ |
| | TKEVPWSFGQGTKVEVK (SEQ ID NO: 28) |
| CD33 | QVQLQQPGAEVVKPGASVKMSCKASGYTFTSYYIHWIKQTPGQGLE |
| huMy9- | WVGVIYPGNDDISYNQKFQGKATLTADKSSTTAYMQLSSLTSEDSA |
| 6 VH | VYYCAREVRLRYFDVWGQGTTVTVSS (SEQ ID NO: 22) |
| CD33 | EIVLTQSPGSLAVSPGERVTMSCKSSQSVFFSSSQKNYLAWYQQIPG |
| huMy9- | QSPRLLIYWASTRESGVPDRFTGSGSGTDFTLTISSVQPEDLAIYYCH |
| 6 VL | QYLSSRTFGQGTKLEIK (SEQ ID NO: 23) |
| CD33 | QVQLQQPGAEVVKPGASVKMSCKASGYTFTSYYIHWIKQTPGQGLE |
| huMy9- | WVGVIYPGNDDISYNQKFQGKATLTADKSSTTAYMQLSSLTSEDSA |
| 6- | VYYCAREVRLRYFDVWGQGTTVTVSSASTKGPSVFPLAPCSRSTSES |
| IgG4- | TAALGCLVKDYFPEPVTVSWNSGALTSGVHTFPAVLQSSGLYSLSSV |
| S228P | VTVPSSSLGTKTYTCNVDHKPSNTKVDKRVESKYGPPCPPCPAPEFL |
| heavy | GGPSVFLFPPKPKDTLMISRTPEVTCVVVDVSQEDPEVQFNWYVDGV |
| chain | EVHNAKTKPREEQFNSTYRVVSVLTVLHQDWLNGKEYKCKVSNKG |
| | LPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGN |
| | VFSCSVMHEALHNHYTQKSLSLSLGK (SEQ ID NO: 24) |
| CD33 | EIVLTQSPGSLAVSPGERVTMSCKSSQSVFFSSSQKNYLAWYQQIPG |
| huMy9- | QSPRLLIYWASTRESGVPDRFTGSGSGTDFTLTISSVQPEDLAIYYCH |
| 6- | QYLSSRTFGQGTKLEIKRTVAAPSVFIFPPSDEQLKSGTASVVCLLNN |
| IgG4- | FYPREAKVQWKVDNALQSGNSQESVTEQDSKDSTYSLSSTLTLSKA |
| S228P | DYEKHKVYACEVTHQGLSSPVTKSFNRGEC (SEQ ID NO: 25) |
| light | |
| chain | |
| PSMA- | QVQLVESGGGLVKPGGSLRLSCAASGFTFSDFYMYWIRQAPGKGLE |
| A VH | WVATISDGGGYTSYPDSVKGRFTISRDNAKNSLYLQMNSLRAEDTA |
| | VYYCARGLWLRDALDYWGQGTTVTVSS (SEQ ID NO: 7) |
| PSMA- | EIVLTQSPATLSLSPGERATLSCSASSSISSNYLHWYQQKPGQAPRLLI |
| A VL | YRTSNLASGIPARFSGSGSGTDYTLTISRLEPEDFAVYYCQQGSYIPF |
| | TFGQGTKLEIK (SEQ ID NO: 8) |
| PSMA- | QVQLVQSGGGLVQPGGSLRLSCAASGFTFSSYWMSWVRQAPGKGL |
| B VH | EWVANIKQDGSEKYYVDSVKGRFTISRDNAKNSLYLQMNSLRAED |
| | TAVYYCARVWDYYYDSSGDAFDIWGQGTMVTVSS (SEQ ID NO: 9) |
| PSMA- | VIWMTQSPSSVSASVGDRVTITCRASQGISSWLAWYQQKPGKAPKL |
| B VL | LIYAASNLQSGVPSRFSGSGSGTDFTLTISSLQPEDFATYYCQQANSF |
| | PLTFGGGTKVDIK (SEQ ID NO: 10) |
| PSMA- | QVQLVESGGGVVQPGRSLRLSCAASGFAFSRYGMHWVRQAPGKGL |
| C VH | EWVAVIWYDGSNKYYADSVKGRFTISRDNSKNTQYLQMNSLRAED |
| | TAVYYCARGGDFLYYYYYGMDVWGQGTTVTVSS (SEQ ID NO: 11) |
| PSMA- | DIQMTQSPSSLSASVGDRVTITCRASQGISNYLAWYQQKTGKVPKFL |
| C VL | IYEASTLQSGVPSRFSGGGSGTDFTLTISSLQPEDVATYYCQNYNSAP |
| | FTFGPGTKVDIK (SEQ ID NO: 12) |
| PSMA- | EVQLQQSGPELVKPGTSVRISCKTSGYTFTEYTIHWVKQSHGKSLE |
| D VH | WIGNINPNNGGTTYNQKFEDKATLTVDKSSSTAYMELRSLTSEDSA |
| | VYYCAAGWNFDYWGQGTTLTVSS (SEQ ID NO: 29) |
| PSMA- | DIVMTQSHKEMSTSVGDRVSIICKASQDVGTAVDWYQQKPGQSPKL |
| D VL | LIYWASTRHTGVPDRFTGSGSGTDFTLAITNVQSEDLADYFCQQYN |
| | SYPLTFGAGTKLEIK (SEQ ID NO: 30) |
| HER2- | EVQLVESGGGLVQPGGSLRLSCAASGFTFTDYTMDWVRQAPGKGL |
| A VH | EWVADVNPNSGGSIYNQRFKGRFTLSVDRSKNTLYLQMNSLRAED |
| | TAVYYCARNLGPSFYFDYWGQGTLVTVSS (SEQ ID NO: 13) |
| HER2- | DIQMTQSPSSLSASVGDRVTITCKASQDVSIGVAWYQQKPGKAPKL |
| A VL | LIYSASYRYTGVPSRFSGSGSGTDFTLTISSLQPEDFATYYCQQYYIY |
| | PYTFGQGTKVEIK (SEQ ID NO: 14) |
| HER2- | EVQLVESGGGLVQPGGSLRLSCAASGFNIKDTYIHWVRQAPGKGLE |
| B VH | WVARIYPTNGYTRYADSVKGRFTISADTSKNTAYLQMNSLRAEDT |
| | AVYYCSRWGGDGFYAMDYWGQGTLVTVSS (SEQ ID NO: 15) |
| HER2- | DIQMTQSPSSLSASVGDRVTITCRASQDVNTAVAWYQQKPGKAPKL |
| B VL | LIYSASFLYSGVPSRFSGSRSGTDFTLTISSLQPEDFATYYCQQHYTT |
| | PPTFGQGTKVEIK (SEQ ID NO: 16) |
| HER2- | QVQLQQSGPELVKPGASLKLSCTASGFNIKDTYIHWVKQRPEQGLE |
| C VH | WIGRIYPTNGYTRYDPKFQDKATITADTSSNTAYLQVSRLTSEDTA |
| | VYYCSRWGGDGFYAMDYWGQGASVTVSS (SEQ ID NO: 17) |
| HER2- | DIVMTQSHKFMSTSVGDRVSITCKASQDVNTAVAWYQQKPGHSPK |
| C VL | LLIYSASFRYTGVPDRFTGSRSGTDFTFTISSVQAEDLAVYYCQQHY |
| | TTPPTFGGGTKVEIK (SEQ ID NO: 18) |
| CD20- | QVQLQQPGAELVKPGASVKMSCKASGYTFTSYNMHWVKQTPGRG |
| A VH | LEWIGAIYPGNGDTSYNQKFKGKATLTADKSSSTAYMQLSSLTSED |
| | SAVYYCARSTYYGGDWYFNVWGAGTTVTVSA (SEQ ID NO: 31) |
| CD20- | QIVLSQSPAILSASPGEKVTMTCRASSSVSYIHWFQQKPGSSPKPWIY |
| A VL | ATSNLASGVPVRFSGSGSGTSYSLTISRVEAEDAATYYCQQWTSNPP |
| | TFGGGTKLEIK (SEQ ID NO: 32) |
| CD79b- | EVQLVESGGGLVQPGGSLRLSCAASGYTFSSYWIEWVRQAPGKGLE |
| A VH | WIGEILPGGGDTNYNEIFKGRATFSADTSKNTAYLQMNSLRAEDTA |
| | VYYCTRRVPIRLDYWGQGTLVTVSS (SEQ ID NO: 33) |
| CD79b- | DIQLTQSPSSLSASVGDRVTITCKASQSVDYEGDSFLNWYQQKPGK |
| B VH | APKLLIYAASNLESGVPSRFSGSGSGTDFTLTISSLQPEDFATYYCQQ |
| | SNEDPLTFGQGTKVEIK (SEQ ID NO: 34) |
| BCMA- | QVQLVQSGAEVKKPGSSVKVSCKASGGTFSNYWMHWVRQAPGQG |
| A | LEWMGATYRGHSDTYYNQKFKGRVTITADKSTSTAYMELSSLRSED |
| heavy | TAVYYCARGAIYDGYDVLDNWGQGTLVTVSSASTKGPSVFPLAPSS |
| chain | KSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKE |
| | YKCKVSNKALPAPIEKTISKAKGQPREPQVYTLPPSRDELTKNQVSLT |
| | CLVKGFYPSDIAVEWESNGQPENNYKTIPPVLDSDGSFFLYSKLTVD |
| | KSRWQQGNVFSCSVMHEALHNHYTQKSLSLSPGK (SEQ ID NO: 35) |
| BCMA-A | DIQMTQSPSSLSASVGDRVTITCSASQDISNYLNWYQQKPGKAPKLLI |
| light | YYTSNLHSGVPSRFSGSGSGTDFTLTISSLQPEDFATYYCQQYRKLPW |
| chain | TFGQGTKLEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC (SEQ ID NO: 36) |

In some aspects, an antibody or antigen binding portion thereof comprises a constant region. A linker can be attached to an amino acid in the constant region. In some aspects, an antibody or antigen binding portion thereof comprises a CH1 domain. A linker can be attached to an amino acid in a CH1 domain. In some aspects, an antibody or antigen binding portion thereof comprises a CH2 domain. A linker can be attached to an amino acid in a CH2 domain. In some aspects, an antibody or antigen binding portion thereof comprises a CH3 domain. A linker can be attached to an amino acid in a CH3 domain. In some aspects, an antibody or antigen binding portion thereof comprises a CL domain. A linker can be attached to an amino acid in a CL domain.

In some aspects, a constant region, a CH1 domain, a CH2 domain, a CH3 domain, or a CL domain is an engineered constant region, CH1 domain, CH2 domain, CH3 domain or a CL domain.

In some aspects, an antibody or antigen binding portion thereof comprises a heavy chain constant region, e.g., a human heavy chain constant region. A linker can be attached to an amino acid in a heavy chain constant region, e.g., a human heavy chain constant region. In some aspects, an antibody or antigen binding portion thereof comprises an IgG heavy chain constant region, e.g., a human IgG heavy chain constant region. A linker can be attached to an amino acid in an IgG heavy chain constant region, e.g., a human IgG heavy chain constant region. In some aspects, an antibody or antigen binding portion thereof comprises an IgG1 heavy chain constant region, e.g., a human IgG1 heavy chain constant region. A linker can be attached to an amino acid in an IgG1 heavy chain constant region, e.g., a human IgG1 heavy chain constant region. In some aspects, an antibody or antigen binding portion thereof comprises an IgG4 heavy chain constant region. A linker can be attached to an amino acid in an IgG4 heavy chain constant region, e.g., a human IgG4 heavy chain constant region.

In some aspects, an antibody or antigen binding portion thereof comprises a light chain constant region, e.g., a human light chain constant region. A linker can be attached to an amino acid in a light chain constant region, e.g., a human light chain constant region. In some aspects, an antibody or antigen binding portion thereof comprises a kappa light chain constant region, e.g., a human kappa light chain constant region. A linker can be attached to an amino acid in a kappa light chain constant region, e.g., a human kappa light chain constant region. In some aspects, an antibody or antigen binding portion thereof comprises a gamma light chain constant region, e.g., a human gamma light chain constant region. A linker can be attached to an amino acid in a gamma light chain constant region, e.g., a human gamma light chain constant region.

In some aspects, an antibody or antigen binding portion thereof comprises an engineered cysteine at heavy chain position S239 according to EU numbering. A linker can be attached to S239C. In some aspects, an antibody or antigen binding portion thereof comprises an engineered cysteine at heavy chain position K334 according to EU numbering. A linker can be attached to K334C.

Accordingly, an antibody or antigen binding portion thereof can comprise a heavy chain constant region of SEQ ID NO:19, SEQ ID NO:20, or SEQ ID NO:21.

IgG1 Heavy Chain Constant Region
(SEQ ID NO: 19)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG
IgG1 Heavy Chain Constant Region S239C
(SEQ ID NO: 20)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPCVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG
IgG1 Heavy Chain Constant Region K334C
(SEQ ID NO: 21)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIECTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

An antibody or antigen binding portion thereof can comprise a heavy chain constant region of SEQ ID NO:26.

IgG4 Heavy Chain Constant Region S228P
(SEQ ID NO: 26)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

An antibody or antigen binding portion thereof can comprise a heavy chain constant region of SEQ ID NO:37.

IgG1 N297A Constant regions (CH1-Hinge-CH2-CH3)
(SEQ ID NO: 37)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

In some aspects, a linker can be attached to heavy chain Q295 of an antibody or antigen binding portion thereof according to EU numbering.

An antibody “which binds” a molecular target or an antigen of interest is one capable of binding that antigen with sufficient affinity such that the antibody is useful in targeting a cell expressing the antigen.

In the present disclosure, group “Bm” can be conjugated to more than one compound that induces protein-protein interaction. In some aspects, “Bm” can be conjugated to from 1 to 10 compounds. In some aspects, “Bm” can be conjugated to from 1 to 9 compounds. In some aspects, “Bm” can be conjugated to from 1 to 8 compounds. In some aspects, “Bm” can be conjugated to 1, 2, 3, 4, 5, 6, 7, 8, 9, or 10 compounds. In some aspects, “Bm” can be conjugated to 7 or 8 compounds. In some aspects, “Bm” is conjugated to 5 compounds. In some aspects, “Bm” is conjugated to 6 compounds s. In some aspects, “Bm” is conjugated to 7 compounds. In some aspects, “Bm” is conjugated to 8 compounds. In some aspects, “Bm” is conjugated to 9 compounds.

### V. Compositions and Methods of Using

The conjugates and/or compounds described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

Pharmaceutical compositions comprising the conjugates described herein may also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream, into muscle, or directly into an organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle injectors, needle-free injectors, and infusion techniques.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

Compositions for parenteral administration can be formulated to be immediate and/or modified release. Modified release formulations include delayed-, sustained-, pulsed-, controlled-, targeted, and programmed release. Thus, the compositions can be formulated as a solid, semi-solid, or thixotropic liquid for administration as an implanted depot providing modified release of the active agent.

The parenteral formulations can be admixed with other suitable pharmaceutically acceptable excipients used in parenteral dosage forms such as, but not limited to, preservatives.

In another aspect, the pharmaceutical compositions can be formulated as suitable oral dosage forms such as tablets, capsules, powders, pellets, suspensions, solutions, emulsions, and the like. Other suitable carriers can be present such as disintegrants, diluents, chelating agents, binders, glidants, lubricants, fillers, bulking agents, anti-adherants, and the like.

Oral dosage formulations may also contain other suitable pharmaceutical excipients such as sweeteners, vehicle/wetting agents, coloring agents, flavoring agents, preservatives, viscosity enhancing/thickening agents, and the like.

The conjugates described herein can be used to treat various cancers. Certain conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament.

The conjugates of the present disclosure can be used as medicaments such as an agents for the prophylaxis or treatment of diseases, for example, cancers e.g., colorectal cancers (e.g., colorectal cancer, rectal cancer, anus cancer, familial colorectal cancer, hereditary nonpolyposis colorectal cancer, gastrointestinal stromal tumor), lung cancers (e.g., non-small-cell lung cancer, small-cell lung cancer, malignant mesothelioma), mesothelioma, pancreatic cancers (e.g., pancreatic ductal carcinoma, pancreatic endocrine tumor), pharynx cancer, larynx cancer, esophageal cancer, stomach/gastric cancers (e.g., papillary adenocarcinoma, mucinous adenocarcinoma, adenosquamous carcinoma), duodenal cancer, small intestinal cancer, breast cancers (e.g., invasive ductal carcinoma, non-invasive ductal carcinoma, inflammatory breast cancer), ovarian cancers (e.g., ovarian epithelial cancer, extragonadal germ cell tumor, ovarian germ cell tumor, ovarian low-malignant potential tumor), testis tumor, prostate cancers (e.g., hormone-dependent prostate cancer, non-hormone dependent prostate cancer, castration-resistant prostate cancer), liver cancers (e.g., hepatocellular cancer, primary liver cancer, extrahepatic bile duct cancer), thyroid cancers (e.g., medullary thyroid carcinoma), renal cancers (e.g., renal cell cancers (e.g., clear cell renal cell cancer), transitional cell cancer of renal pelvis and ureter), uterine cancers (e.g., cervical cancer, uterine body cancer, uterus sarcoma), gestational choriocarcinoma, brain tumors (e.g., medulloblastoma, glioma, pineal astrocytic tumors, pilocytic astrocytoma, diffuse astrocytoma, anaplastic astrocytoma, pituitary adenoma), retinoblastoma, skin cancers (e.g., basalioma, malignant melanoma), sarcomas (e.g., rhabdomyosarcoma, leiomyosarcoma, soft tissue sarcoma, spindle cell sarcoma), malignant bone tumor, bladder cancer, hematological/blood cancers (e.g., multiple myeloma, leukemias (e.g., acute myelogenous leukemia), malignant lymphoma, Hodgkin's disease, chronic myeloproliferative disease), cancer of unknown primary; a cancer growth inhibitor; a cancer metastasis inhibitor; an apoptosis promoter; an agent for the treatment of precancerous lesions (e.g., myelodysplastic syndromes); and the like.

In certain aspects, conjugates of the present disclosure can be used as a medicament for breast cancer, gastric cancer, ovarian cancer, uterine cancer, lung cancer, pancreatic cancer, liver cancer, lymphoma, or hematological cancers. In certain aspects, conjugates of the present disclosure can be used as a medicament for prostate cancer, breast cancer, gastric cancer, non-small cell lung cancer, bile duct cancer, colon cancer, ovarian cancer, or neuregulin-1 (NRG1)-positive cancer. In certain aspects, conjugates of the present disclosure can be used as a medicament for a non-Hodkin lymphoma (NHL), e.g., a B-cell non-Hodgkin lymphoma or a diffuse large B-cell lymphoma (DLBCL).

Furthermore, conjugates of the present disclosure or can be used concurrently with a non-drug therapy. To be precise, the conjugates can be combined with a non-drug therapy such as (1) surgery, (2) hypertensive chemotherapy using angiotensin II etc., (3) gene therapy, (4) thermotherapy, (5) cryotherapy, (6) laser cauterization and (7) radiotherapy.

For example, by using a conjugate of the present disclosure before or after the above-mentioned surgery and the like, effects such as prevention of emergence of resistance, prolongation of Disease-Free Survival, suppression of cancer metastasis or recurrence, prolongation of life and the like may be afforded.

In addition, it is possible to combine a treatment with conjugates of the present disclosure with a supportive therapy: (i) administration of antibiotic (e.g., β-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a pharmaceutical agent for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a pharmaceutical agent for suppressing multiple drug resistance of cancer and the like.

In some aspects, conjugate of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the conjugates of the disclosure in combination with one or more additional therapeutic agents. In some aspects, the conjugates of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Additional example of immune checkpoint inhibitors include T-cell immunoglobulin and ITIM domain (TIGIT) antagonists, V-domain Ig suppressor of T-cell activation (VISTA) antagonists, B and T cell lymphocyte attenuator (BTLA) antagonists, and lymphocyte activation gene-3 (LAG-3) antagonists. A comprehensive and non-limiting list of combination treatment is disclosed in detail in the Combination Treatments section of this application.

In some aspects, the conjugate of the disclosure is administered to the subject prior to or after the administration of the additional therapeutic agent. In other aspects, the conjugate of the disclosure is administered to the subject concurrently with the additional therapeutic agent. In certain aspects, the conjugate of the disclosure and the additional therapeutic agent can be administered concurrently as a single composition in a pharmaceutically acceptable carrier. In other aspects, the conjugate of the disclosure and the additional therapeutic agent are administered concurrently as separate compositions.

In some aspects, a subject that can be treated with the conjugate of the present disclosure is a nonhuman animal such as a rat or a mouse. In some aspects, the subject that can be treated is a human.

### VI. Methods of Preparing Compound and Conjugates

The present disclosure provides a method of preparing the conjugates, the method comprising reacting a binding moiety with a compound of formula (XXII):

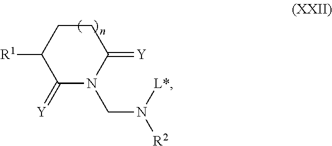

or a pharmaceutically acceptable salt thereof, wherein:
n is 0 or 1;
R¹ is a compound that induces a protein-protein interaction;
R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;

each Y is independently S or O; and

L* is a cleavable linker precursor that conjugates to the binding moiety.

The present disclosure also provides a method of a preparing a compound of formula (XXXII):

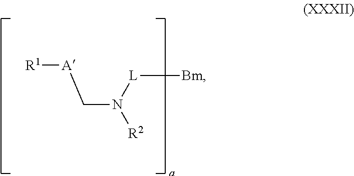

or a pharmaceutically acceptable salt thereof, wherein:
a is from 1 to 10;
A′ is

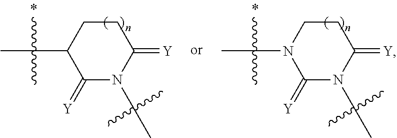

wherein
   n is 0 or 1;
   each Y is independently S or O;
   [symbol] indicates the point of attachment to R¹; and
   [symbol] indicates the point of attachment to the methylene group;
R¹, together with A′, is a compound that induces a protein-protein interaction;
R² is selected from hydrogen, a group that provides stability to R¹-A′, a group that provides solubility to R¹-A, and a group that provides stability and solubility to R¹-A′;
L is a cleavable linker; and
Bm is a binding moiety that is capable of specifically binding to a protein; the method comprising:
reacting a compound of (XXXI)

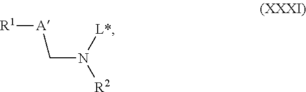

or a pharmaceutically acceptable salt thereof, wherein:
A′, R¹, and R² are as defined above and
L* is a cleavable linker precursor;
   with a binding moiety that is capable of specifically binding to a protein.

As described herein, the linker precursor contains a heterobifunctional group that connects to the binding moiety.

In some aspects, the linker precursor is cleavable by a protease. In some aspects, the linker precursor is selected from the group consisting of

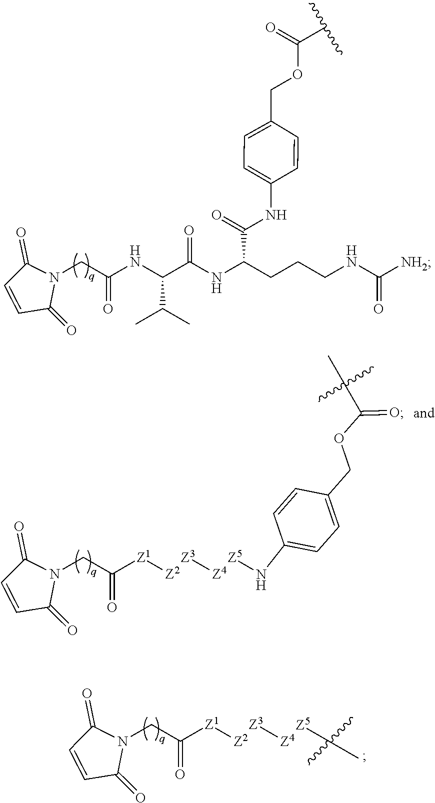

wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, and Z⁴ are amino acid residues; and
[symbol] is the point of attachment to the parent molecular moiety.
Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, and Z⁴, and Z⁵ are amino acid residues.

In some aspects, Z¹ is absent or glycine; Z² is absent or selected from the group consisting of L-glutamine, D-glutamine, L-glutamic acid, D-glutamic acid, L-aspartic acid, D-aspartic acid, L-alanine, D-alanine, and glycine; Z³ is selected from the group consisting of L-valine, D-valine, L-alanine, D-alanine, L-phenylalanine, D-phenylalanine, and glycine; Z⁴ is selected from the group consisting of L-citrulline, D-citrulline, L-asparagine, D-asparagine, L-lysine, D-lysine, L-phenylalamine, D-phenylalanine, and glycine; and Z⁵ is absent or glycine.

In some aspects, L* is

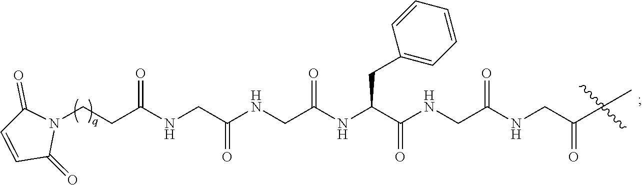

wherein [symbol] is the point of attachment to the parent molecular moiety.

In some aspects, q is 4.

In some aspects, L* is a bioreducible linker precursor. In some aspects, the bioreducible linker precursor is selected from the group consisting of

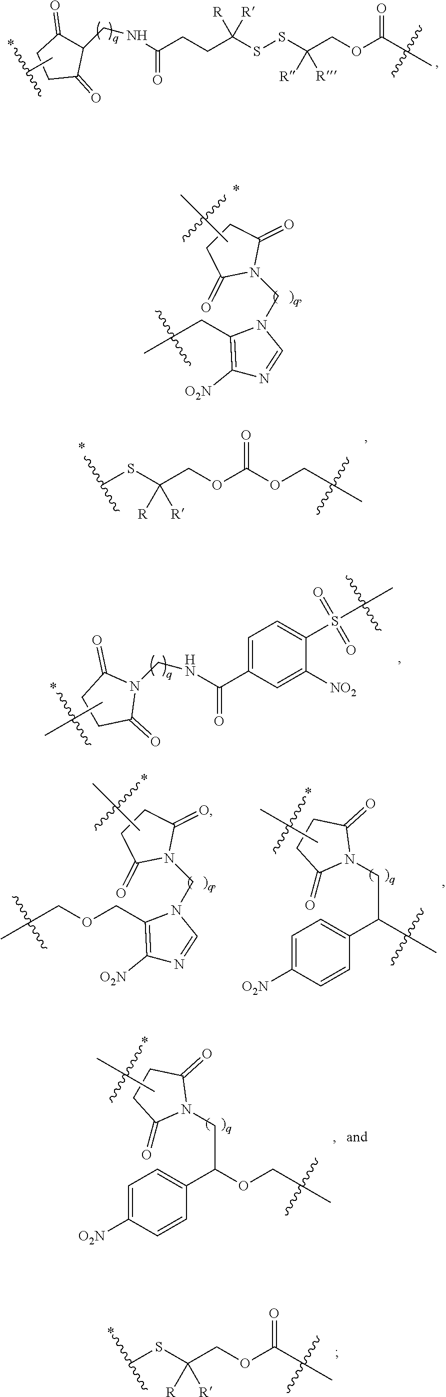

wherein:
q is from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; and
[symbol] is the point of attachment to the parent molecular moiety.

In some aspects, L* is a bioreducible linker which is

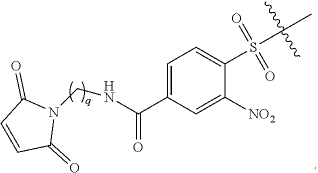

In certain aspects, L* is a click-to-release linker precursor. In some aspects, L* is

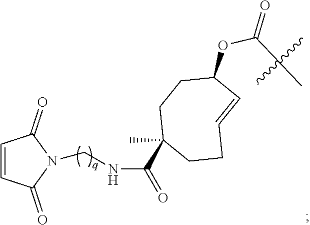

wherein:
   q is from 2 to 10; and
   [symbol] is the point of attachment to the parent molecular moiety.

In certain aspects, L* is a beta-glucuronidase cleavable linker precursor. In some aspects, L* is

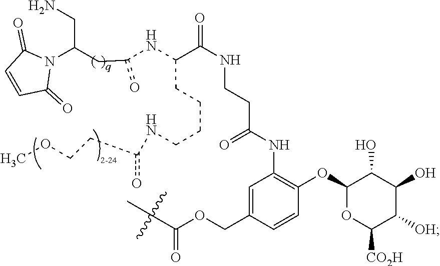

wherein:
   q is from 2 to 10;
   - - - is absent or a bond; and
   [symbol] is the point of attachment to the parent molecular moiety.

In certain aspects, R² is a group that provides stability to the conjugate. In some aspects, R² is selected from C₂-C₆alkenyl, C₁-C₆alkyl; C₂-C₆alkynyl, benzyl, C₃-C₆cycloalkyl, and C₃-C₆cycloalkyl(C₁-C₃alkyl). In some aspects, R² is C₁-C₆alkyl. In some aspects, R² is methyl.

In certain aspects, R² is a group that provides solubility to the conjugate. In some aspects, R² is selected from:

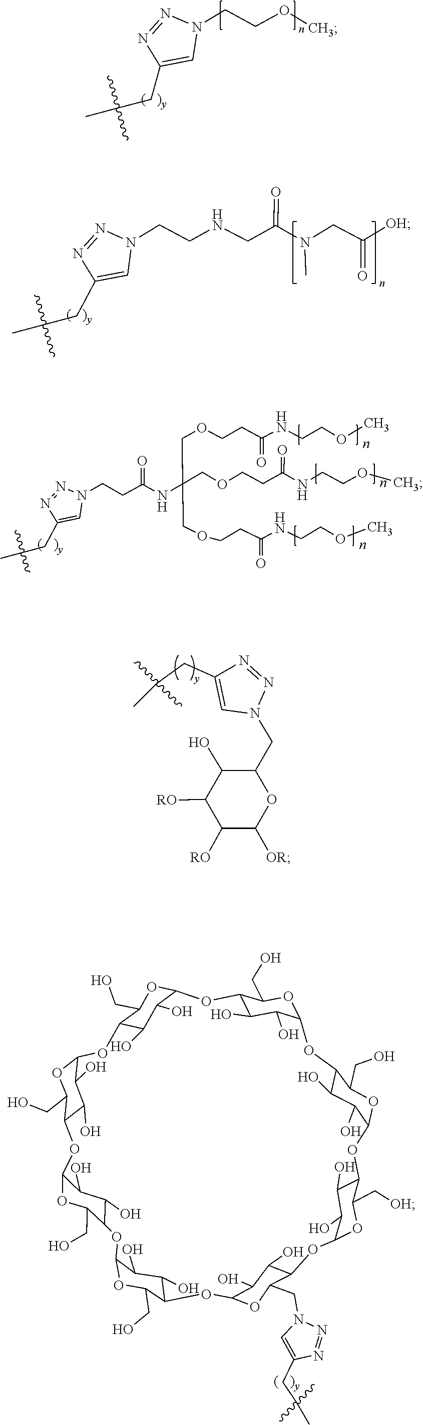

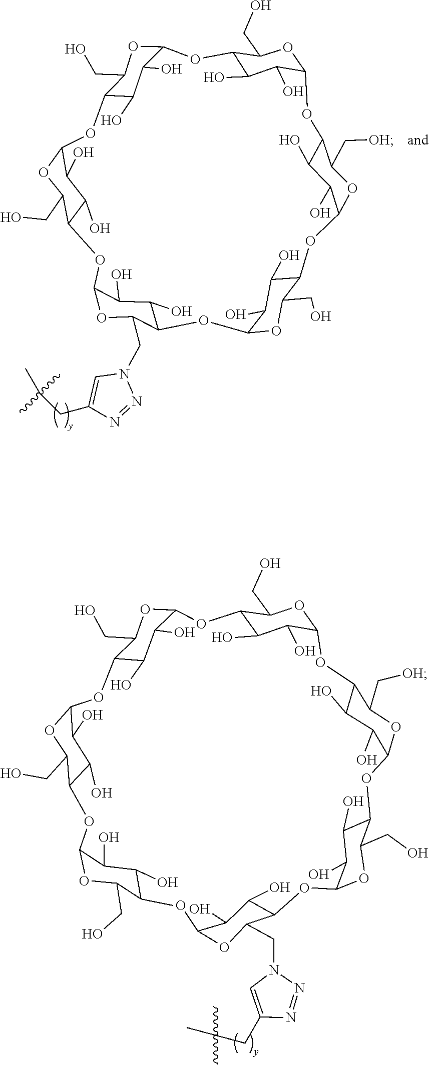

wherein:
each n is independently 1, 2, 3, 4, or 5;
each y is independently 1 or 2; and
each R is independently hydrogen, C₆H₁₁O₅, C₁₂H₂₁O₁₀, C₁₈H₃₁O₁₅, or C₂₄H₄₁O₂₀. In some aspects. R¹ is a compound of formula (XXX):

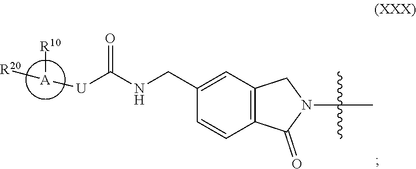

wherein:
[symbol] denotes the point of attachment to the parent molecular moiety;
A is phenyl or a C₄-C₁₀cycloalkyl ring;
R¹⁰ is independently selected from hydrogen and halo;
U is selected from NH and CF₂; and
R²⁰ is selected from —C(O)R³, —N(R⁴)₂, —(CH₂)ₙOH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
R³ is hydrogen or C₁-C₆alkyl;
each R⁴ is independently hydrogen or C₁-C₆alkyl;
Q′ is O, S, or NR⁴;
n is 1-6; and
m is 2-5.
In some aspects,
A is phenyl;
U is NH;
R¹⁰ is halo; and
R²⁰ is methyl.

In some aspects, A is phenyl;
U is NH;
R¹⁰ is halo; and
R²⁰ is —(CH₂)₂O(CH₂)₂NHCH₃.

In some aspects, the compound of formula (XXX) is a compound selected from the group consisting of:

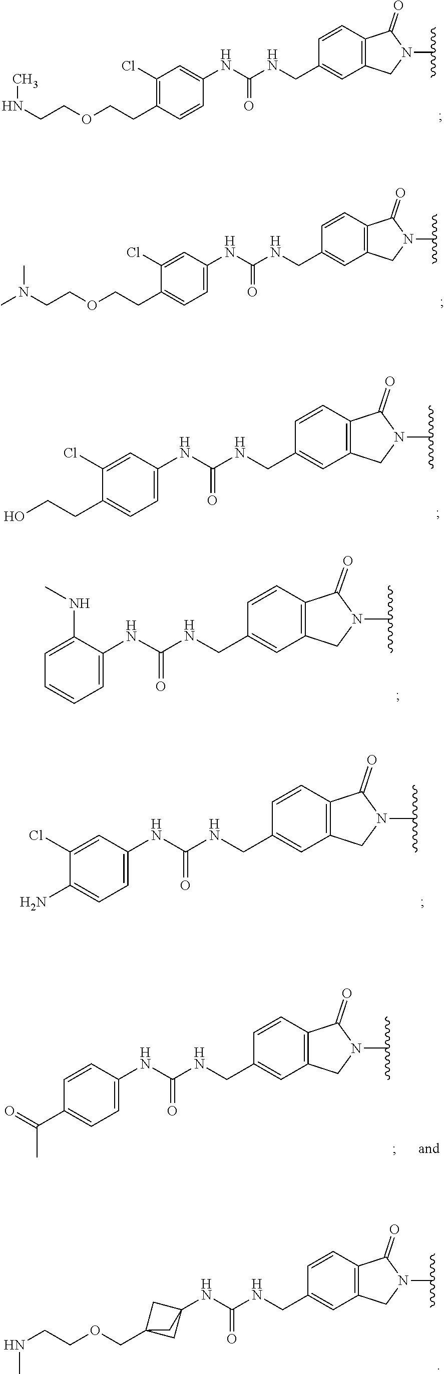

In some aspects, R¹-A′ is a proteolysis targeting chimera (PROTAC). In certain aspects, R¹ has the formula:

POI-L¹⁰⁰-CBN;

wherein:
POI is a compound that binds to a protein of interest;
L¹⁰⁰ is a PROTAC linker; and
CBN is a cereblon binding moiety.

In some aspects, the protein of interest is a nuclear hormone receptor, a translation termination factor, a transcription factor, a cyclin-dependent kinase, a tyrosine kinase, a serine/threonine kinase, or an E3 ligase. In some aspects, the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

In certain aspects, L¹⁰⁰ comprises one or more functional groups selected from glycol, alkyl, alkynyl, triazolyl, piperazinyl, piperidinyl, and combinations thereof.

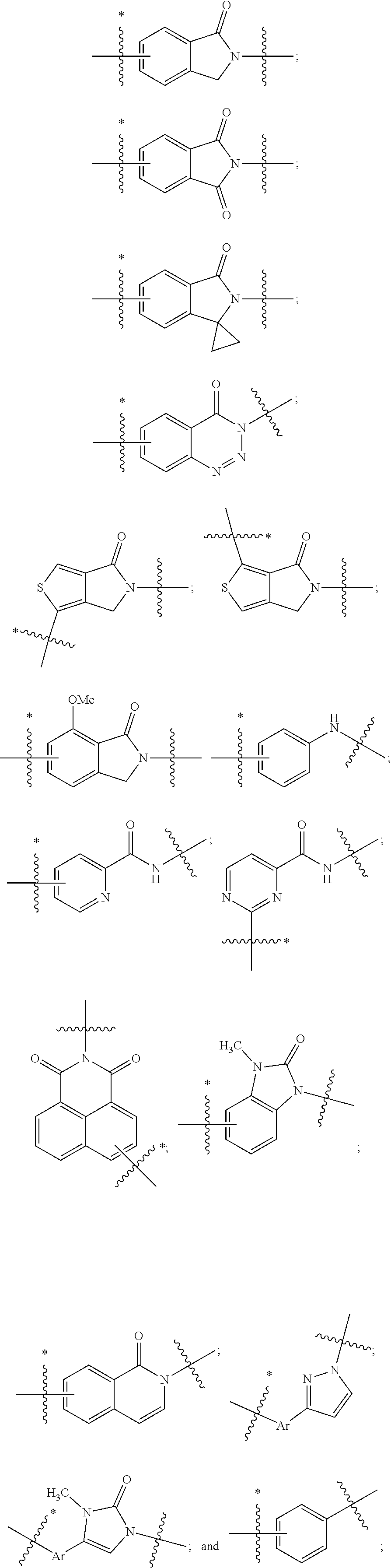

In some aspects, CBN is selected from
wherein
[symbol] indicates the point of attachment to A′; and
[symbol] indicates the point of attachment to L¹⁰⁰.

In certain embodiments, R¹ is selected from

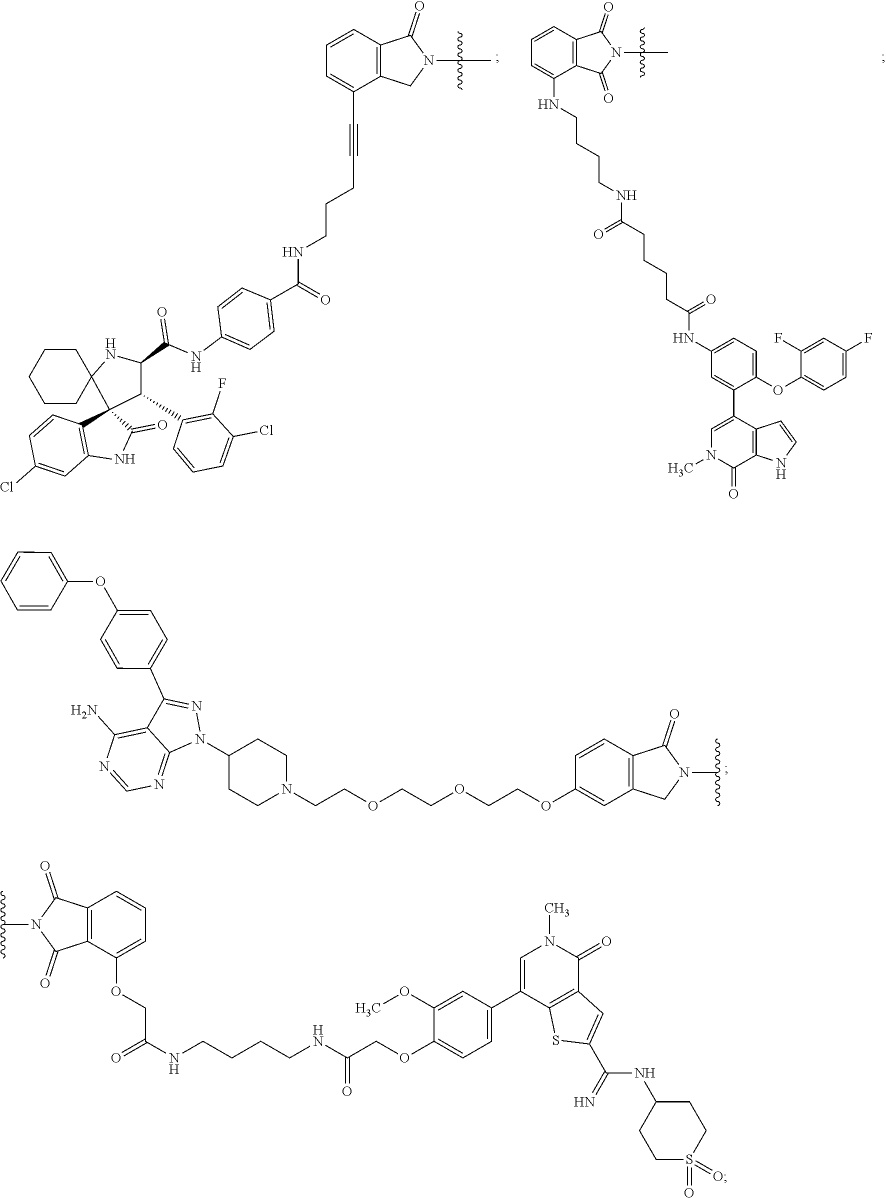

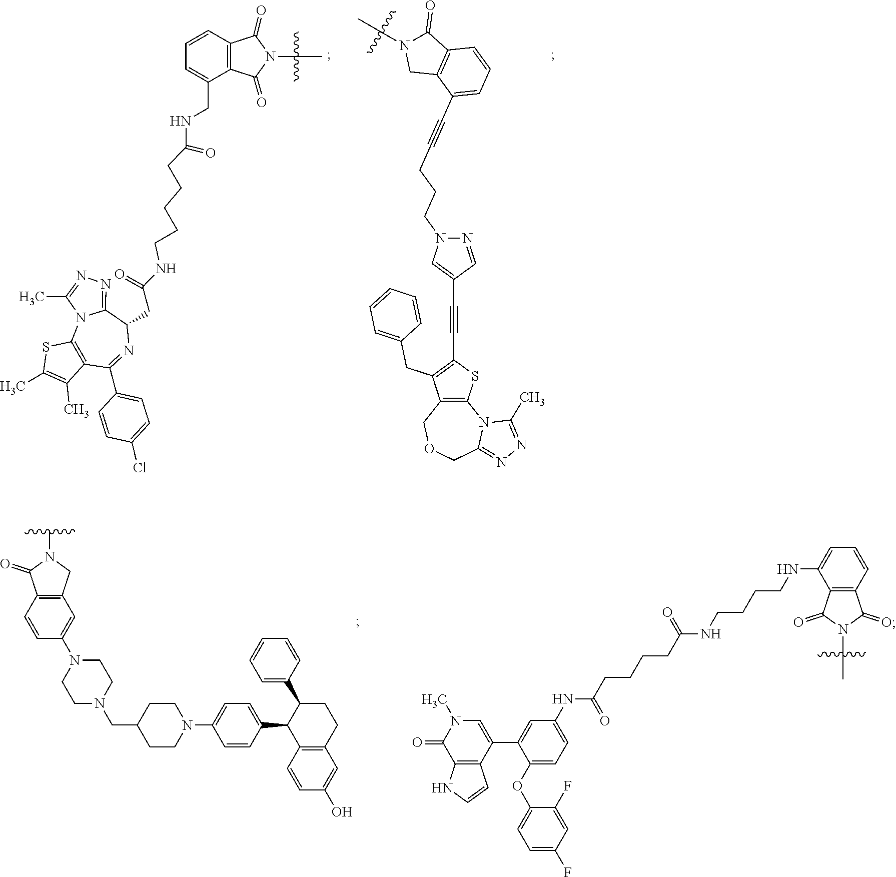

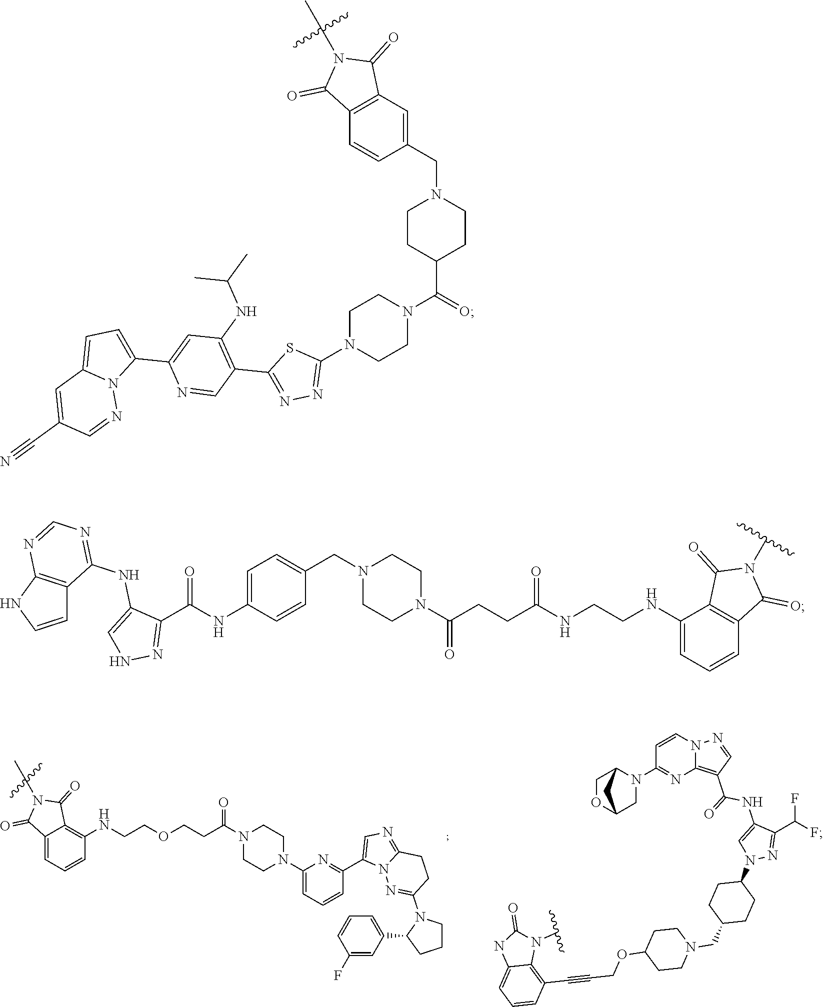

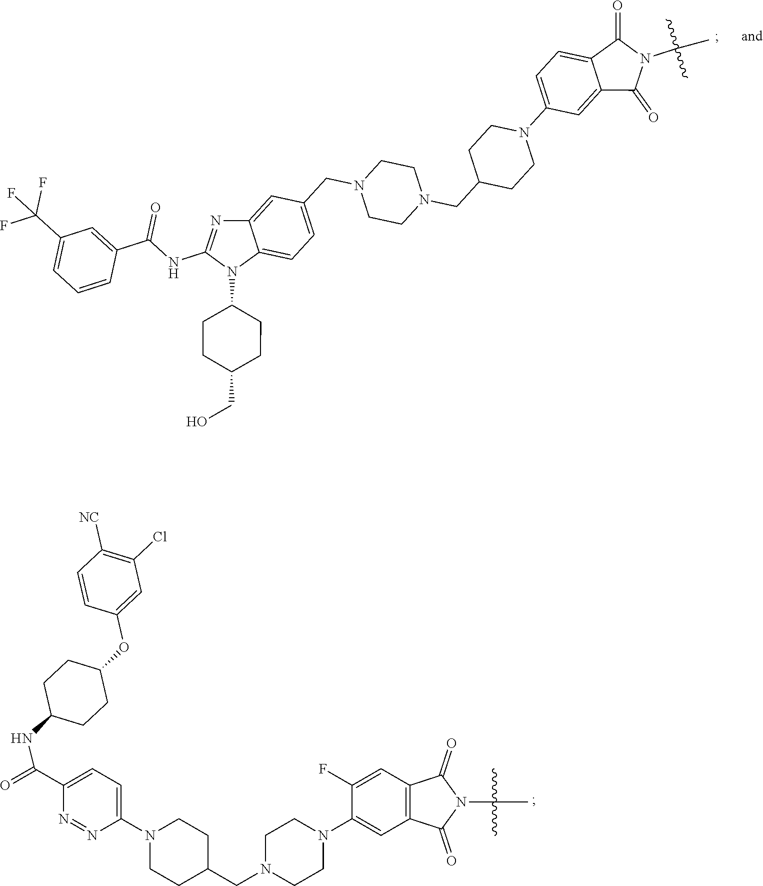

wherein [symbol] denotes the point of attachment to A′.

In some aspects, the binding moiety is pre-treated before it is reacted with the compound of formula (XXII) or (XXXI). In certain aspects, the compound of formula (XXII) or (XXXI) is reacted with a binding moiety, which comprises an antibody or an antigen binding portion thereof. In aspects where the binding moiety is an antibody, the antibody can be pretreated to reduce interchain disulfides prior to reaction with the compound of formula (XXII) or (XXXI).

General methods for conjugating the compounds of Formula (XXII) or Formula (XXXI) to a cysteine in the Bm through a maleimide component of the linker is shown in Scheme I-I. R¹, R², and Y are defined herein and L** is a portion of a linker as defined herein.

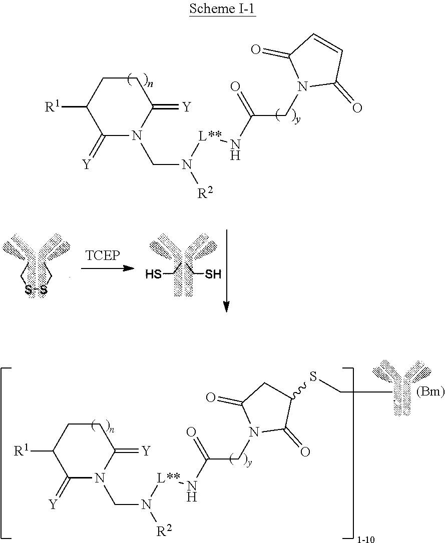

General methods for conjugating the compounds of Formula (XXII) or Formula (XXXI) to a lysine in the Bm through an N-hydroxysuccinimide component of the linker is shown in Scheme I-2. R¹, R^{Z}, and Y are defined herein and L** is a portion of a linker as defined herein.

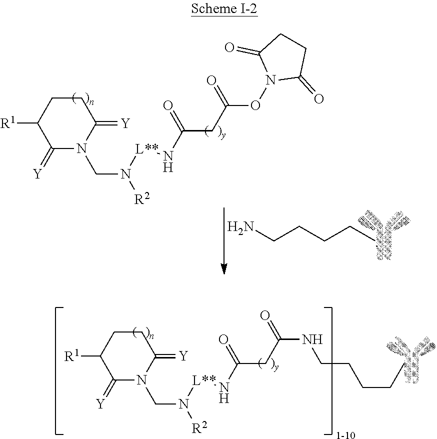

General methods for preparing the compounds of Formula (XX) and Formula (XXX) and activation within the cell to release the compound that induces a protein-protein interaction are shown in Scheme I-3. R¹, R², and Y are defined herein and L** is a portion of a linker as defined herein.

In step 1, the heterobifunctional group within the linker is activated. In step 2, the protected linker is attached to the nitrogen of ring A. Step 3 shows deprotection of the amine and step 4 shows attachment of the Bm group and step 5 shows conjugation of Bm to the PPI-linker moiety (which is shown in Schemes I-1 and I-2). Step 6 depicts the activation of the conjugate in the cancer cell through a retro-Mannich reaction which releases the active compound that induces a protein-protein interaction.

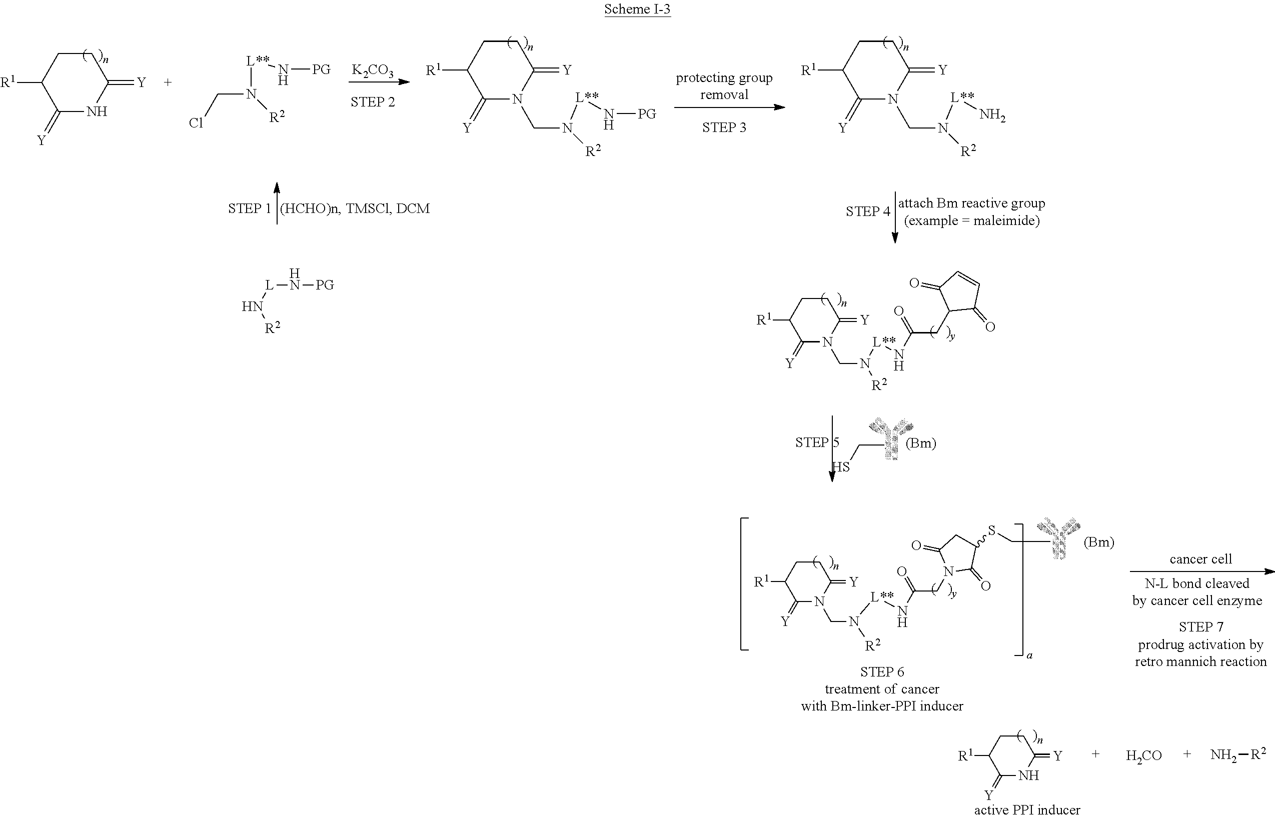

### EXAMPLES

### General Synthetic Methods and Intermediates

The compounds of the present disclosure can be prepared by one of ordinary skill in the art in light of the present disclosure and knowledge in the art, and/or by reference to the schemes shown below and the synthetic examples. Some reagents and intermediates are known in the art. Other reagents and intermediates can be made by methods known in the art using readily available materials. Exemplary synthetic routes are set forth in Schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

Abbreviations used in the schemes generally follow conventions used in the art. Chemical abbreviations used in the specification and examples are defined as follows:

“Et₃N” and “TEA” for trimethylamine; “DMF” for N,N-dimethylformamide; “r.t.” or “rt” or “RT” for room temperature or retention time (context will dictate); “h” for hours; “min” for minutes; “CDI” for 1,1′-carbonyldiimidazole; “DMAP” for N,N-dimethylaminopyridine; “TBAI” for tetrabutyl ammonium bromide; “HATU” for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; “DIEA” and “iPrNEt₂” for diisopropylethylamine; “ACN” for acetonitrile; “DCM” for dichlormethane; “MeOH” for methanol; “Me” for methyl; “PE” for petroleum ether; “TFA” for trifluoroacetic acid; “BOC” or “floe” “DMSO” for dimethylsulfoxide; “Cbz” for carbobenzyloxy; “EtOH” for ethanol; “HOBt” or “HOBT” for 1-hydroxybenzotriazole hydrate; “NBS” for N-bromosuccinimide; “TMS” for trimethylsilyl; and “THF” for tetrahydrofuran;

### Example 1: Preparation of Compounds Compound

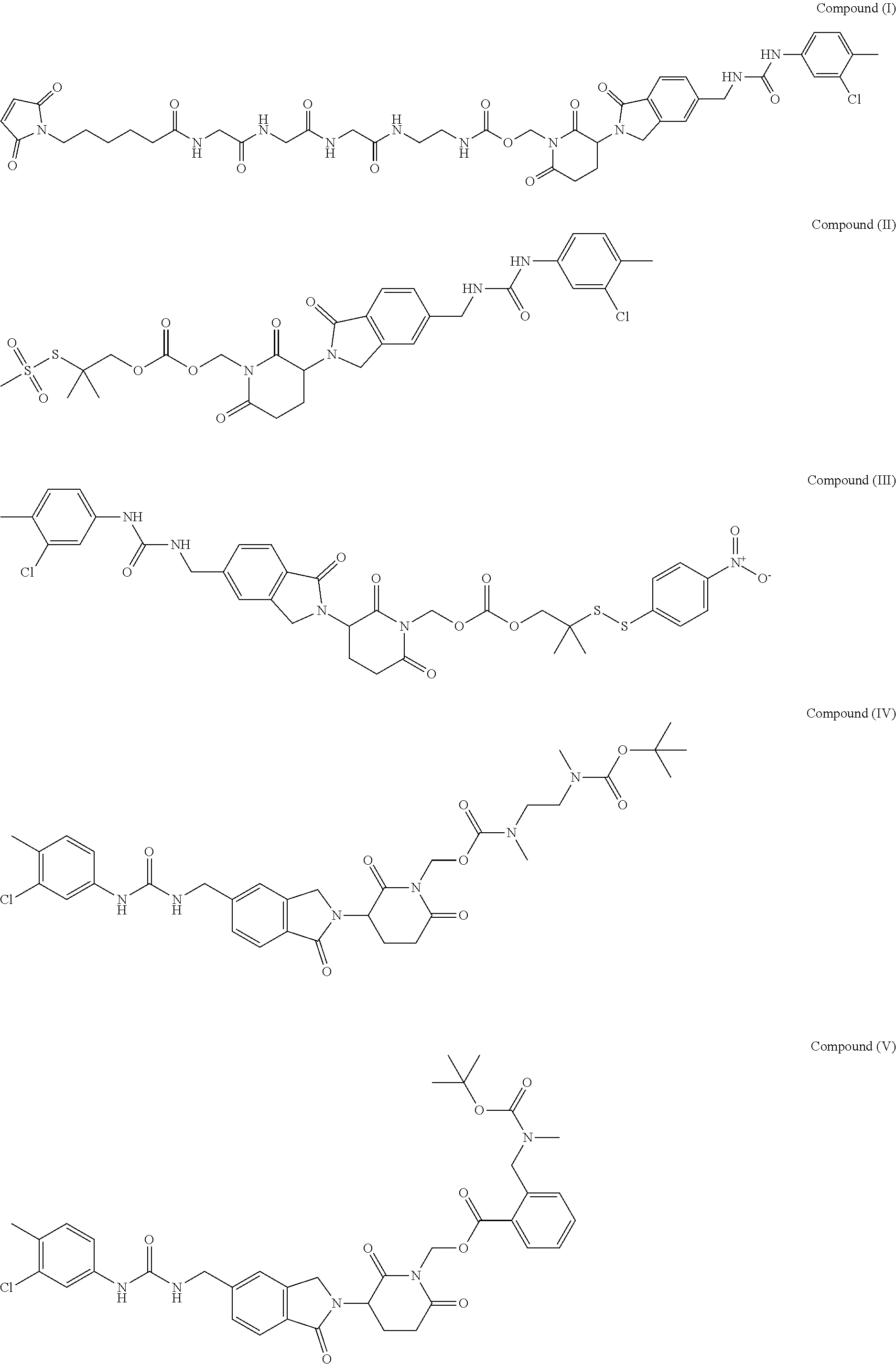

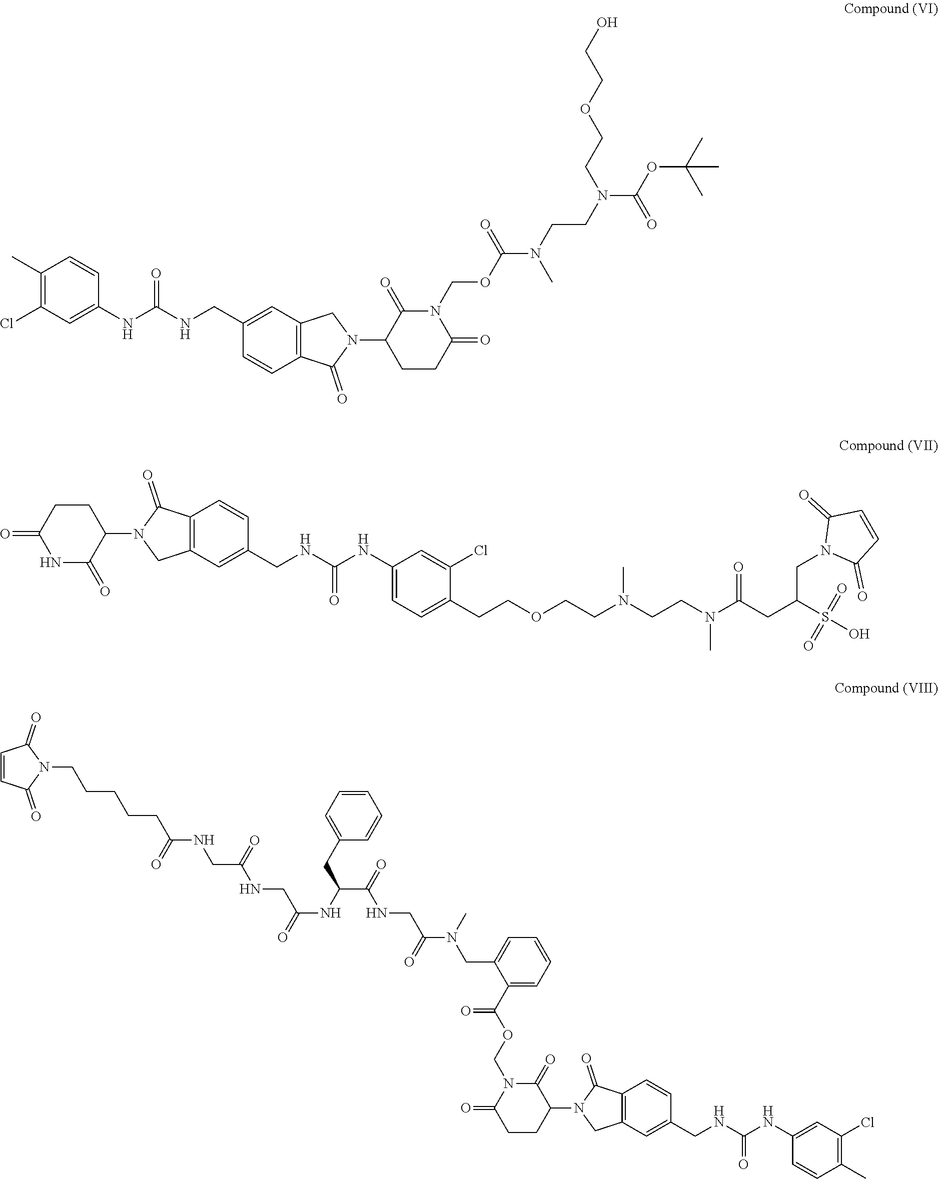 [SMILES: Cc1ccc(NC(=O)NCc2ccc3c(c2)CN(C2CCC(=O)N(COC(=O)N(C)CCN(CCOCCO)C(=O)OC(C)(C)C)C2=O)C3=O)cc1Cl ; CN(CCOCCc1ccc(NC(=O)NCc2ccc3c(c2)CN(C2CCC(=O)NC2=O)C3=O)cc1Cl)CCN(C)C(=O)CC(CN1C(=O)C=CC1=O)S(=O)(=O)O ; Cc1ccc(NC(=O)NCc2ccc3c(c2)CN(C2CCC(=O)N(COC(=O)c4ccccc4CN(C)C(=O)CNC(=O)[C@H](Cc4ccccc4)NC(=O)CNC(=O)CNC(=O)CCCCCN4C(=O)C=CC4=O)C2=O)C3=O)cc1Cl]

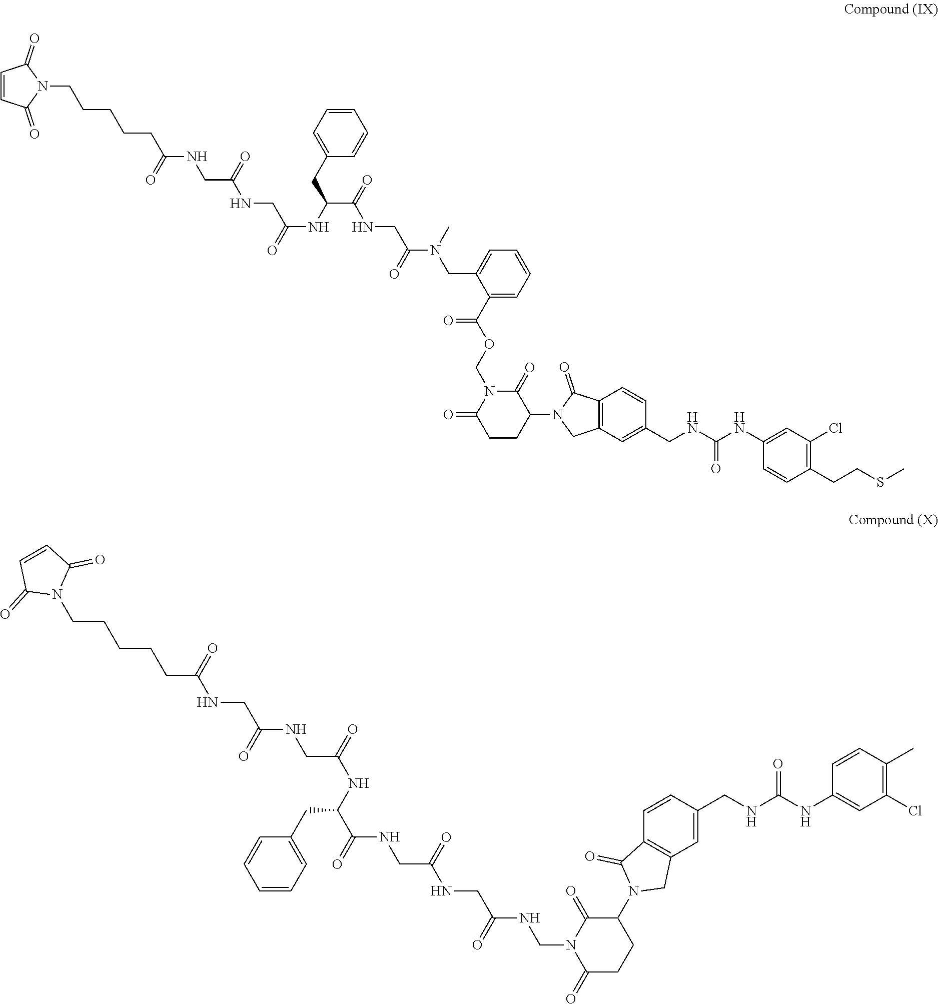 [SMILES: CSCCc1ccc(NC(=O)NCc2ccc3c(c2)CN(C2CCC(=O)N(COC(=O)c4ccccc4CN(C)C(=O)CNC(=O)[C@H](Cc4ccccc4)NC(=O)CNC(=O)CNC(=O)CCCCCN4C(=O)C=CC4=O)C2=O)C3=O)cc1Cl ; Cc1ccc(NC(=O)NCc2ccc3c(c2)CN(C2CCC(=O)N(CNC(=O)CNC(=O)CNC(=O)[C@H](Cc4ccccc4)NC(=O)CNC(=O)CNC(=O)CCCCCN4C(=O)C=CC4=O)C2=O)C3=O)cc1Cl]

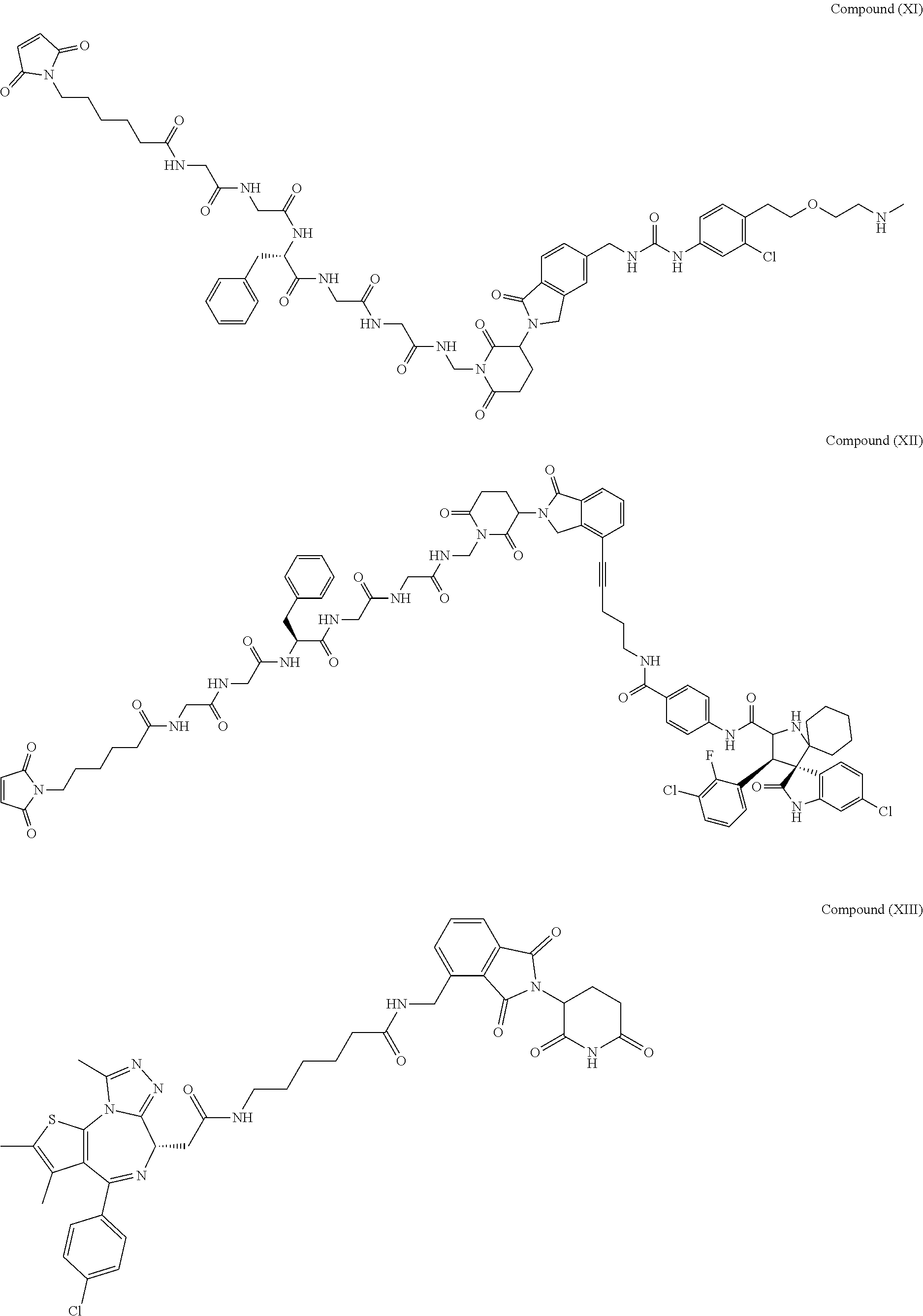

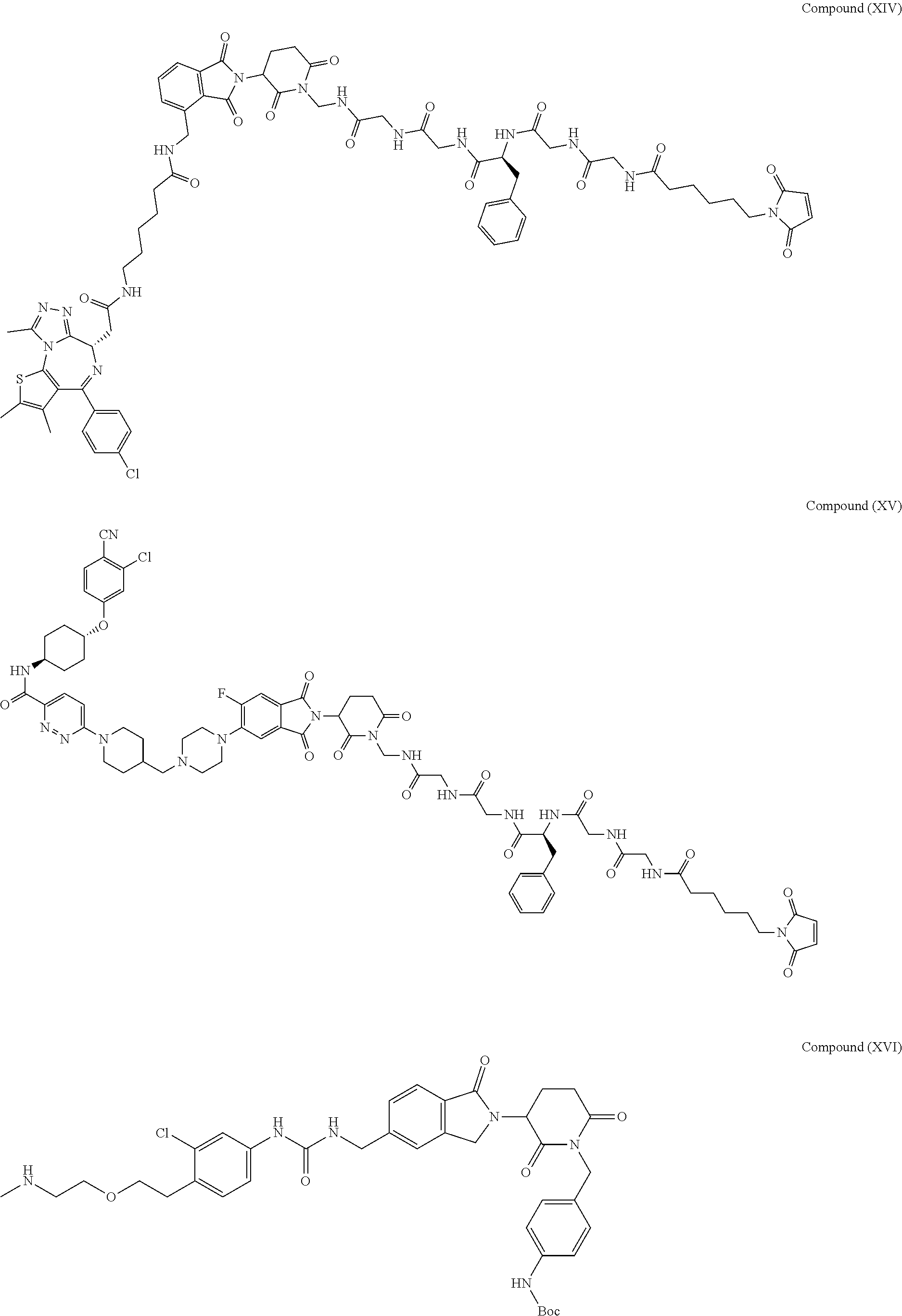 [SMILES: Cc1sc2c(c1C)C(c1ccc(Cl)cc1)=N[C@@H](CC(=O)NCCCCCC(=O)NCc1cccc3c1C(=O)N(C1CCC(=O)N(CNC(=O)CNC(=O)CNC(=O)[C@H](Cc4ccccc4)NC(=O)CNC(=O)CNC(=O)CCCCCN4C(=O)C=CC4=O)C1=O)C3=O)c1nnc(C)n1-2 ; N#Cc1ccc(O[C@H]2CC[C@H](NC(=O)c3ccc(N4CCC(CN5CCN(c6cc7c(cc6F)C(=O)N(C6CCC(=O)N(CNC(=O)CNC(=O)CNC(=O)[C@H](Cc8ccccc8)NC(=O)CNC(=O)CNC(=O)CCCCCN8C(=O)C=CC8=O)C6=O)C7=O)CC5)CC4)nn3)CC2)cc1Cl ; CNCCOCCc1ccc(NC(=O)NCc2ccc3c(c2)CN(C2CCC(=O)N(Cc4ccc(NC(=O)OC(C)(C)C)cc4)C2=O)C3=O)cc1Cl]

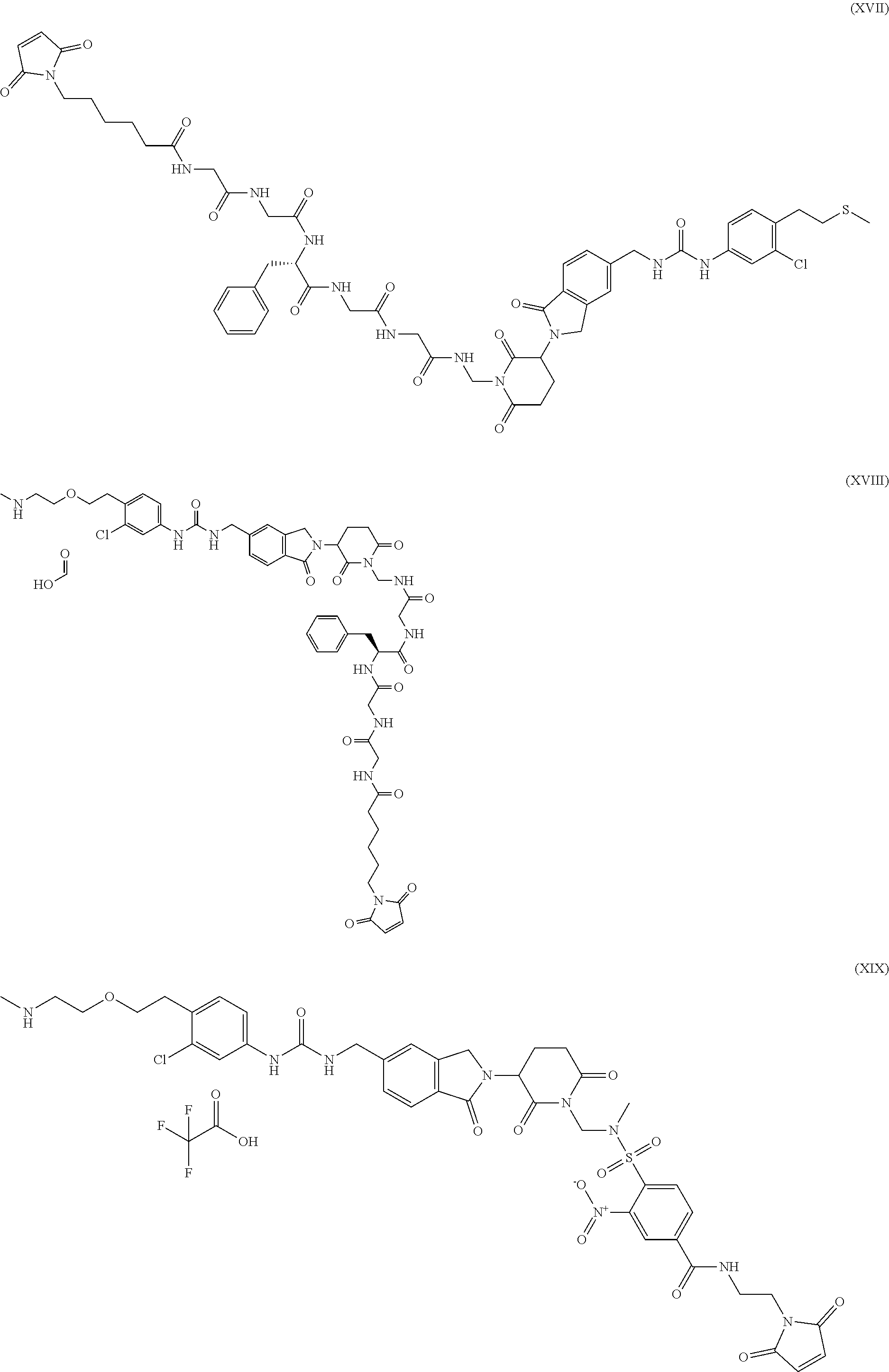

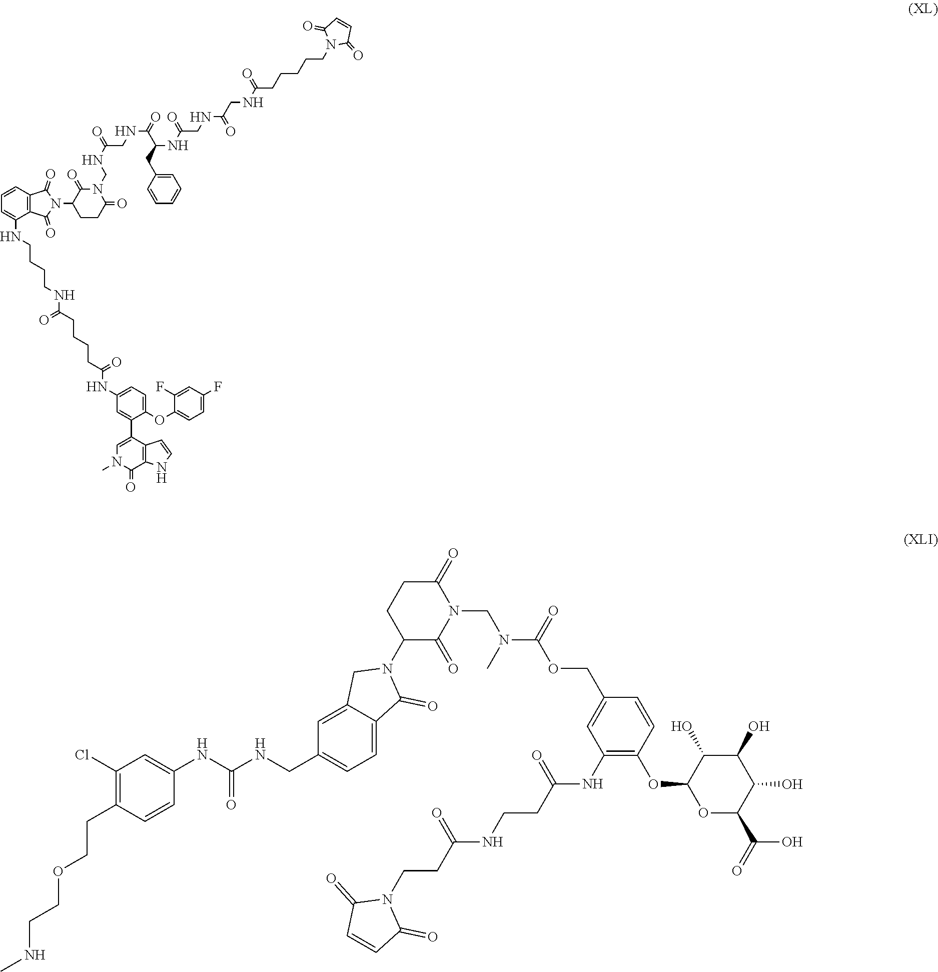 [SMILES: Cn1cc(-c2cc(NC(=O)CCCCC(=O)NCCCCNc3cccc4c3C(=O)N(C3CCC(=O)N(CNC(=O)CNC(=O)[C@H](Cc5ccccc5)NC(=O)CNC(=O)CNC(=O)CCCCCN5C(=O)C=CC5=O)C3=O)C4=O)ccc2Oc2ccc(F)cc2F)c2cc[nH]c2c1=O ; CNCCOCCc1ccc(NC(=O)NCc2ccc3c(c2)CN(C2CCC(=O)N(CN(C)C(=O)OCc4ccc(O[C@@H]5O[C@H](C(=O)O)[C@@H](O)[C@H](O)[C@H]5O)c(NC(=O)CCNC(=O)CCN5C(=O)C=CC5=O)c4)C2=O)C3=O)cc1Cl]

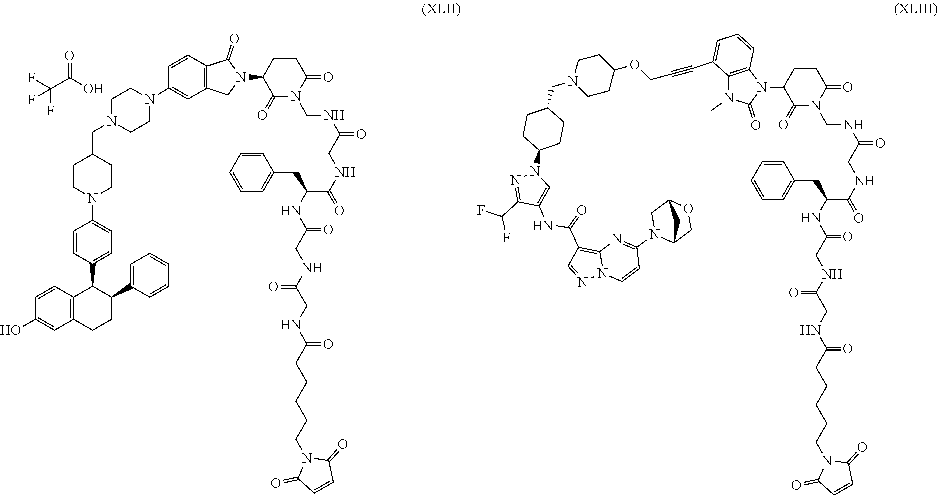

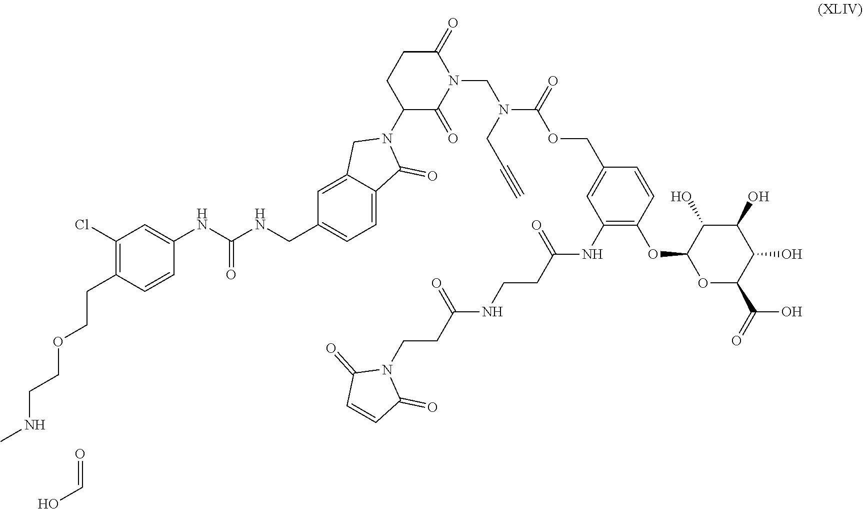 [SMILES: C#CCN(CN1C(=O)CCC(N2Cc3cc(CNC(=O)Nc4ccc(CCOCCNC)c(Cl)c4)ccc3C2=O)C1=O)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1 ; O=CO]

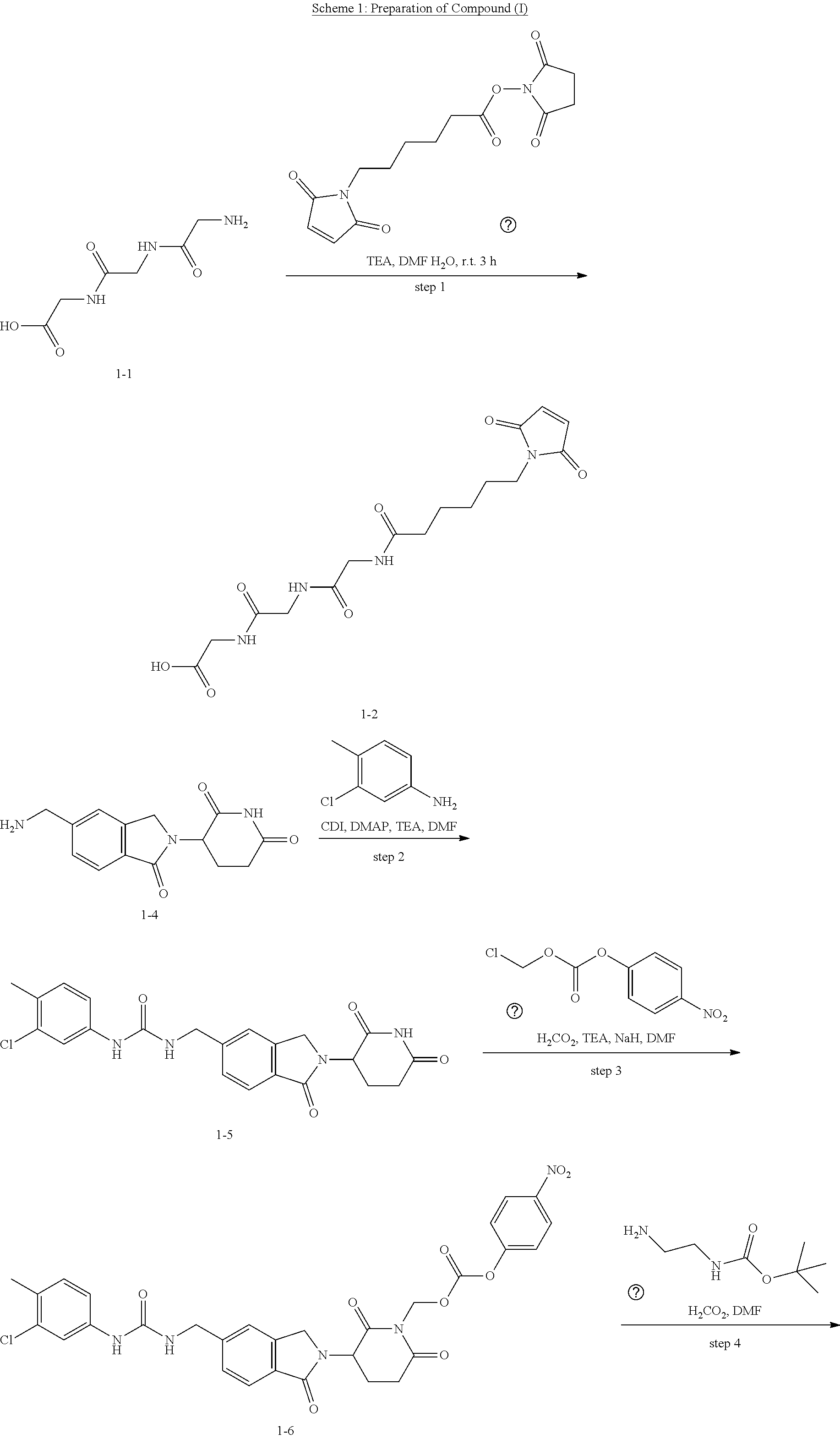

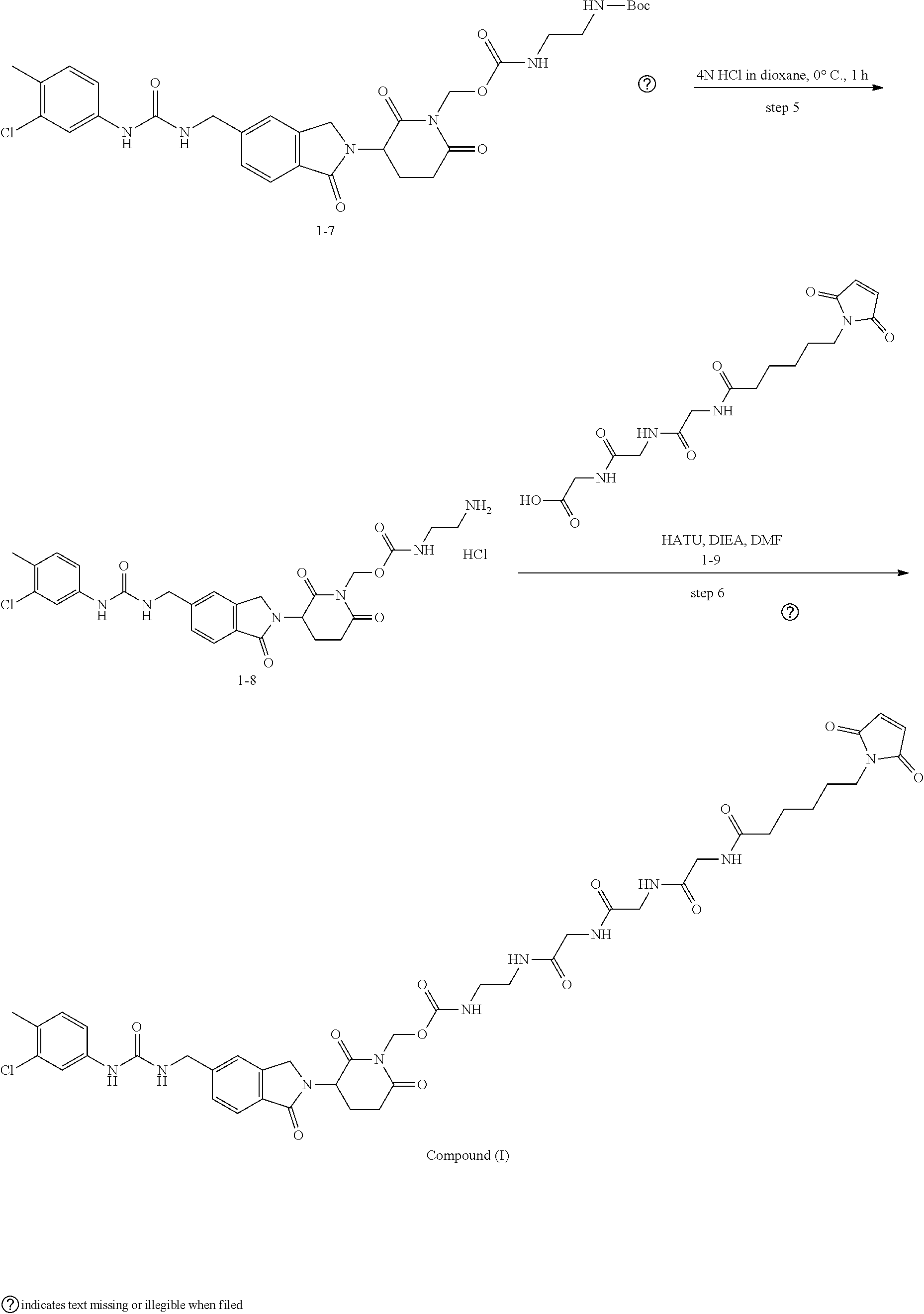 [SMILES: Cc1ccc(NC(=O)NCc2ccc3c(c2)CN(C2CCC(=O)N(COC(=O)NCCNC(=O)OC(C)(C)C)C2=O)C3=O)cc1Cl ; Cc1ccc(NC(=O)NCc2ccc3c(c2)CN(C2CCC(=O)N(COC(=O)NCCN)C2=O)C3=O)cc1Cl ; Cl ; O=C(O)CNC(=O)CNC(=O)CNC(=O)CCCCCN1C(=O)C=CC1=O ; Cc1ccc(NC(=O)NCc2ccc3c(c2)CN(C2CCC(=O)N(COC(=O)NCCNC(=O)CNC(=O)CNC(=O)CNC(=O)CCCCCN4C(=O)C=CC4=O)C2=O)C3=O)cc1Cl]

### Step 1. Synthesis of Compound 1-3

To a stirred mixture of Gly-Gly-Gly (Compound 1-1, 5.00 g, 25.1 mmol, 1.00 equiv) in H₂O (25 mL) was added TEA (7.6 g, 75.1 mmol, 2.99 equiv) dropwise at 0° C. To the above mixture was added 2,5-dioxopyrrolidin-1-yl 6-(2,5-dioxopyrrol-1-yl)hexanoate (Compound 1-2, 9.29 g, 30.1 mmol, 1.20 equiv) in DMF (25 mL) dropwise at 0° C. The resulting mixture was stirred for additional 3 h at room temperature. LCMS indicated the reaction was completed. The mixture was acidified to pH 4 with HCl (2N, aq.). The residue was purified by reverse flash chromatography with the following conditions: column, C18 silica gel (330 g, 20-40 um); mobile phase, water (containing 0.05% TFA), ACN (0% to 20% gradient in 30 min); detector, UV 220 nm. This provided (2-[2-[6-(2,5-dioxopyrrol-1-yl)hexanamido]acetamido]-acetamido)acetic acid (Compound 1-3, 1 g, 8%) as a white solid. LCMS (ES, m/s): 383 [M+H]⁺

### Step 1A: Synthesis of Compound 1-4

### Step a: 1:3-(5-bromo-1-oxoisoindolin-2-yl)piperidine-2,6-dione

To a stirred mixture of methyl 4-bromo-2-(bromomethyl)benzoate (60.0 g, 194.8 mmol, 1.00 equiv) and 3-aminopiperidine-2,6-dione hydrochloride (38.48 g, 233.8 mmol, 1.20 equiv) in DMF (120 ml) was added TEA (67.70 mL, 669.0 mmol, 2.50 equiv) dropwise at 25° C. under nitrogen atmosphere. The mixture was stirred at 25° C. for 16 h. This was follow by addition of H₂O (120 mL), AcOH (46 mL) and Et₂O (120 mL) in sequence at 25° C. The mixture was stirred at 25° C. for 2 h. LCMS indicated the reaction was completed. The precipitated solids were collected by filtration and washed with Et₂O (60 mL). This resulted in 3-(5-bromo-1-oxo-3H-isoindol-2-yl)piperidine-2,6-dione (40.0 g, 63%) as a white solid. LCMS (ESI, ms): 323,325 (M+H)⁺. ¹H NMR (300 MHz, DMSO-d₆) δ 11.00 (s, 1H), 7.90 (d, J=1.5 Hz, 1H), 7.74-7.66 (m, 2H), 5.15-5.10 (m, 1H), 4.51-4.32 (m, 2H), 2.93-2.85 (m, 1H), 2.74-2.56 (m, 1H), 2.43-2.32 (m, 1H), 2.06-1.99 (m, 1H).

### Step b: 2-(2,6-dioxopiperidin-3-yl)-1-oxoisoindoline-5-carbonitrile

To a stirred solution of 3-(5-bromo-1-oxo-3H-isoindol-2-yl)piperidine-2,6-dione (2.00 g, 6.19 mmol, 1.00 equiv) in DMF (40.00 mL) were added Zn(CN)₂ (872 mg, 7.42 mmol, 1.20 equiv) and Pd(PPh₃)₄ (715 mg, 0.62 mmol, 0.10 equiv) in portions at room temperature under nitrogen atmosphere. The resulting mixture was stirred for overnight at 80° C. under nitrogen atmosphere. LCMS indicated the reaction was completed. The mixture was allowed to cool down to room temperature. The mixture was added into water (120.00 mL) and were stirred for 30 min. The precipitated solids were collected by filtration and washed with water (3×20 mL) and EtOAc (3×20 mL). The resulting solid was dried under sunlight lamp. This resulted in 2-(2,6-dioxopiperidin-3-yl)-1-oxo-3H-isoindole-5-carbonitrile (1.2 g, 72%) as a white solid. LCMS (ESI, ms): 270 (M+H)⁺; ¹H NMR (400 MHz, DMSO-d₆) δ 11.03 (s, 1H), 8.17 (d, J=1.6 Hz, 1H), 8.00-7.91 (m, 2H), 5.18-5.13 (m, 1H), 4.57-4.40 (m, 2H), 2.92-2.88 (m, 1H), 2.74-2.56 (m, 1H), 2.48-2.37 (m, 1H), 2.07-1.99 (m, 1H).

### Step c: 3-(5-(aminomethyl)-1-oxoisoindolin-2-yl)piperidine-2,6-dione hydrochloride

To a slurry of 2-(2,6-dioxopiperidin-3-yl)₁-oxo-3H-isoindole-5-carbonitrile (8.00 g, 29.7 mmol, 1.00 equiv) in MeOH (67.00 mL) were added HCl (12M, 9.60 mL) and PtO₂ (3.30 g, 14.5 mmol, 0.49 equiv) at 25° C. The mixture was stirred at room temperature for 16 h under hydrogen atmosphere using a hydrogen balloon. LCMS indicated the reaction was completed. The reaction was filtered and washed with MeOH (2×30 mL). The filtrate was evaporated to dryness under reduced pressure. The resulting solid was washed with DCM:MeOH (3:1) (3×30 mL). This resulted in 3[5-(aminomethyl)-1-oxo-3H-isoindol-2-yl]piperidine-2,6-dione hydrochloride (5.08 g, 57%) as a white solid. LCMS (ESI, ms): 274 (M+H−HCl)⁺; ¹H NMR (400 MHz, CD₃OD) δ 7.88 (d, J=8.0 Hz, 1H), 7.68 (s, 1H), 7.62 (d, J=8.0 Hz, 1H), 5.19-5.15 (m, 1H), 4.55-4.53 (m, 2H), 4.26 (s, 2H), 2.95-2.88 (m, 1H), 2.81-2.80 (m, 1H), 2.55-2.45 (m, 1H), 2.22-2.16 (m, 1H).

### Step 2. Synthesis of Compound 1-5

To a stirred mixture of 3-[5-(aminomethyl)-1-oxo-3H-isoindol-2-yl]piperidine-2,6-dione (Compound 1-4, 1.00 g, 3.66 mmol, 1.00 equiv) in DMF (10.00 mL) were added CDI (0.59 g, 3.66 mmol, 1.00 equiv) and TEA (0.37 g, 3.66 mmol, 1.00 equiv) in portions at 0° C. under nitrogen atmosphere. The resulting mixture was stirred for 2 h at 0° C. under nitrogen atmosphere. To the above mixture was added DMAP (1.34 g, 10.98 mmol, 3.00 equiv) and 3-chloro-p-toluidine (0.52 g, 3.66 mmol, 1.00 equiv) in portions at room temperature. The resulting mixture was stirred for overnight at 60° C. under nitrogen atmosphere. LCMS indicated the reaction was completed. The reaction was quenched with water/ice at room temperature. The precipitated solids were collected by filtration and washed with DCM and water. This provided 1-(3-chloro-4-methylphenyl)-3-[[2-(2,6-dioxopiperidin-3-yl)-1-oxo-3H-isoindol-5-yl]methyl]urea (Compound 1-5, 1.0 g, 51%) as a light brown solid. LCMS (ES, m/s): 441,443 [M+1]⁺.

### Step 3. Synthesis of Compound 1-6

To a stirred mixture of 1-(3-chloro-4-methylphenyl)-3-[[2-(2,6-dioxopiperidin-3-yl)-1-oxo-3H-isoindol-5-yl]methyl]urea (Compound 1-5, 500.00 mg, 1.13 mmol, 1.00 equiv) in DMF (5.00 mL) was added K₂CO₃ (500.0 mg, 3.62 mmol, 3.19 equiv) in portions at 0° C. under nitrogen atmosphere. The resulting mixture was stirred for 30 min at 0° C. under nitrogen atmosphere. To the above mixture was added TBAI (100.0 mg, 0.27 mmol, 0.24 equiv), NaI (200.0 mg, 1.34 mmol, 1.18 equiv) and chloromethyl 4-nitrophenyl carbonate (801.0 mg, 3.46 mmol, 3.05 equiv) in portions at 0° C. The resulting mixture was stirred for additional 1 h at 0° C. in dark. LCMS indicated the reaction was completed. The reaction mixture was used to next step without any treatment. LCMS (ES, m/s): 636,638 [M+H]⁺

### Step 4. Synthesis of Compound 1-7

To a stirred mixture of [3-[5-([[(3-chloro-4-methylphenyl)carbamoyl]amino]methyl)-1-oxo-3H-isoindol-2-yl]-2,6-dioxopiperidin-1-yl]methyl 4-nitrophenyl carbonate (Compound 1-6, 500 mg, 0.78 mmol, 1.00 equiv) and K₂CO₃ (500 mg, 3.62 mmol, 4.60 equiv) in DMF (5.00 mL) were added tert-butyl N-(2-aminoethyl)carbamate (400 mg, 2.50 mmol, 3.18 equiv) in portions at 0° C. under nitrogen atmosphere. The resulting mixture was stirred for 1 h at room temperature. LCMS indicated the reaction was completed. The reaction was quenched with water. The resulting mixture was extracted with diethyl ether (3×20 mL). The combined organic layers were washed with brine (20 mL), dried over anhydrous Na₂SO₄. After filtration, the filtrate was concentrated under reduced pressure. The residue was purified by Prep-TLC (EA) to afford [3-[5-([[(3-chloro-4-methylphenyl)carbamoyl]amino]methyl)-1-oxo-3H-isoindol-2-yl]-2,6-dioxopiperidin-1-yl]methyl N-[2-[(tert-butoxycarbonyl)amino]ethyl]-carbamate (Compound 1-7, 270 mg, 44%) as a white solid. LCMS (ES, m/s): 657,659 [M+H]⁺.

### Step 5. Synthesis of Compound 1-8

A mixture of [3-[5-([[(3-chloro-4-methylphenyl)carbamoyl]amino]methyl)-1-oxo-3H-isoindol-2-yl]-2,6-dioxopiperidin-1-yl]methyl N-[2-[(tert-butoxycarbonyl)amino]ethyl]-carbamate (Compound 1-7, 100 mg, 0.13 mmol, 1.00 equiv) in HCl (gas) in 1,4-dioxane (4 M) (1.5 mL) was stirred for for 30 min at 0° C. LCMS indicated the reaction was completed. The resulting mixture was concentrated under reduced pressure. This resulted in [3-[5-([[(3-chloro-4-methylphenyl)carbamoyl]amino]methyl)-1-oxo-3H-isoindol-2-yl]-2,6-dioxopiperidin-1-yl]methyl N-(2-aminoethyl)carbamate hydrochloride (Compound 1-8, 100 mg, 61%) as a white solid. LCMS (ES, m/s): 557,559 [M+H]⁺.

### Step 6. Synthesis of Compound (I)

To a stirred mixture of (2-[2-[6-(2,5-dioxopyrrol-1-yl)hexanamido]acetamido]-acetamido)acetic acid (Compound 1-3, 64 mg, 0.17 mmol, 1.10 equiv) in DMF (3.5 mL) was added HATU (69 mg, 0.18 mmol, 1.20 equiv) in portions at 0° C. The resulting mixture was stirred for min at 25° C. To the above mixture was added [3-[5-([[(3-chloro-4-methylphenyl)carbamoyl]amino]methyl)-1-oxo-3H-isoindol-2-yl]-2,6-dioxopiperidin-1-yl]methyl N-(2-aminoethyl)carbamate hydrochloride (Compound 1-8, 100 mg, 0.15 mmol, 1.00 equiv) and DIEA (49 mg, 0.37 mmol, 2.50 equiv) at 0° C. The resulting mixture was stirred for additional 16 h at 25° C. LCMS indicated the reaction was completed. The crude product was purified by Prep-HPLC with the following conditions: Column, XSelect CSH Prep C18 OBD Column, 19×250 mm, 5 um; mobile phase, water (containing 0.05% TFA) and ACN (24% to 43% in 7 min); Detector, UV 254 nm. The collected fraction was lyophilized to afford [3-[5-([[(3-chloro-4-methylphenyl)carbamoyl]amino]methyl)-1-oxo-3H-isoindol-2-yl]-2,6-dioxopiperidin-1-yl]methyl N-[2-[2-(2-[2-[6-(2,5-dioxopyrrol-1-yl)hexanamido]-acetamido]acetamido)acetamido]ethyl]carbamate (Compound (I), 18.2 mg, 12%). LCMS (ES, m/z): 921,923 [M+H]⁺. ¹H-NMR (CD₃OD, 400 MHz) δ (ppm): 7.76 (d, J=7.6 Hz, 1H), 7.56-7.48 (m, 3H), 7.13-7.12 (m, 2H), 6.76 (s, 2H), 5.75-5.71 (m, 2H), 5.25-5.20 (m, 1H), 4.51-4.46 (m, 4H), 3.85-3.80 (m, 6H), 3.46-3.42 (m, 2H), 3.28-3.26 (m, 2H), 3.23-3.21 (m, 2H), 3.08-2.86 (m, 2H), 2.66-2.39 (m, 1H), 2.27-2.21 (m, 6H), 1.61-1.51 (m, 4H), 1.28-1.24 (m, 2H).

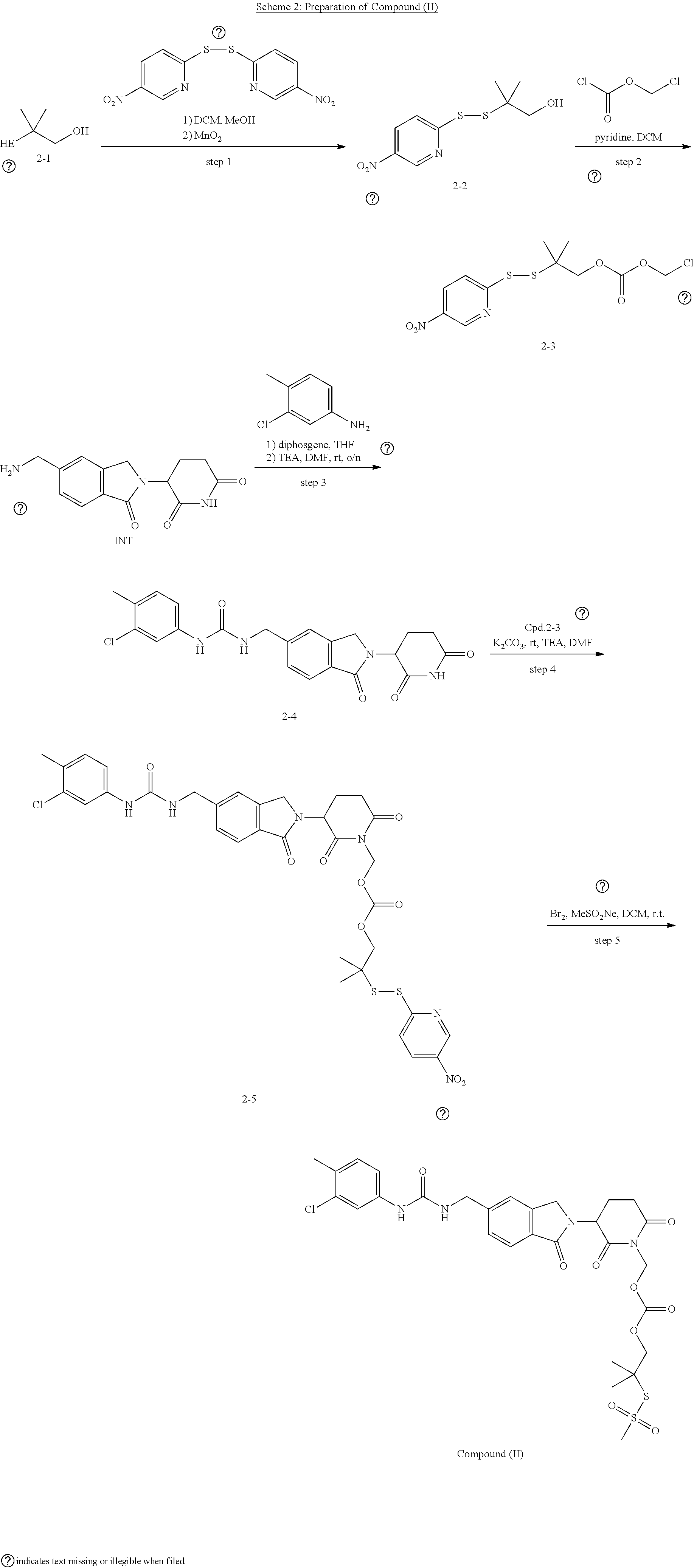

### Step 1. Synthesis of Compound 2-2

To a stirred mixture of 2-methyl-2-sulfanylpropan-1-ol (Compound 2-1, 1.00 g, 9.41 mmol, 1.00 equiv) in DCM (3.00 mL) and MeOH (3.00 mL) was added 5-nitro-2-[(5-nitropyridin-2-yl)disulfanyl]pyridine (1.46 g, 4.70 mmol, 0.50 equiv) at room temperature. The resulting mixture was stirred for overnight at room temperature. To the above mixture was added MnO2 (1.50 g, 17.25 mmol, 1.83 equiv) in portions over at room temperature. The resulting mixture was stirred for additional 2 h at room temperature. LCMS indicated the reaction was completed. The resulting mixture was filtered, the filter cake was washed with DCM (20 mL). The filtrate was concentrated under reduced pressure. The residue was purified by silica gel column chromatography, eluted with PE/EtOAc (8:1) to afford 2-methyl-2-[(5-nitropyridin-2-yl)disulfanyl]propan-1-ol (Compound 2-2, 1.4 g, 57%) as an orange solid. LCMS (ESI, ns): 261 [M+H]+

### Step 2. Synthesis of Compound 2-3

To a stirred mixture of 2-methyl-2-[(5-nitropyridin-2-yl)disulfanyl]propan-1-ol (Compound 2-2, 1.20 g, 4.61 mmol, 1.00 equiv) and pyridine (0.90 g, 11.38 mmol, 2.47 equiv) in DCM (20.00 mL) were added chloromethyl chloroformate (0.60 g 4.65 mmol. 1.01 equiv) in DCM (2.00 mL) dropwise at 0° C. The resulting mixture was stirred for overnight at room temperature. 30% desired product was detected by LCMS. The reaction was quenched with water/ice. The resulting mixture was extracted with DCM (3×20 mL). The combined organic layers were washed with brine (20 mL), dried over anhydrous Na2SO4. After filtration, the filtrate was concentrated under reduced pressure. The residue was purified by silica gel column chromatography, eluted with PE/EtOAc (10:1) to afford chloromethyl 2-methyl-2-[(5-nitropyridin-2-yl)disulfanyl]propyl carbonate (Compound 2-3, 330 mg, 20%) as a light yellow oil. LCMS (ESI, ms): 353,355 [M+H]+

### Step 3. Synthesis of Compound. 2-4

To a stirred mixture of 3-chloro-p-toluidine (102 mg, 0.72 mmol, 0.99 equiv) in THE (10.00 mL) was added diphosgene (145.00 mg, 0.73 mmol, 1.00 equiv) dropwise at 0° C. under nitrogen atmosphere. The resulting mixture was stirred for 1 h at room temperature under nitrogen atmosphere. The resulting mixture was concentrated under reduced pressure. To a stirred mixture of 3-[5-(aminomethyl)-1-oxo-3H-isoindol-2-yl]piperidine-2,6-dione (INT, prepared according to the procedure described for Compound 1-4, 200 mg, 0.73 mmol, 1.00 equiv) and TEA (61.00 mg, 0.60 mmol, 0.82 equiv) in DMF (10.00 mL) were added the above mixture in DMF (15.00 mL) dropwise at 0° C. under nitrogen atmosphere. The resulting mixture was stirred for 3 h at room temperature under nitrogen atmosphere. LCMS indicated the reaction was completed. The residue was purified by reverse flash chromatography with the following conditions: column, C18 silica gel; mobile phase, ACN in water (0.1% FA), 0% to 80% gradient in 40 min; detector, UV 254 nm. This resulted in 1-(3-chloro-4-methylphenyl)-3-[[2-(2,6-dioxopiperidin-3-yl)-1-oxo-3H-isoindol-5-yl]methyl]urea (8Compound 2-45 mg, 26.34%) as a white solid. The product was purified by Prep-HPLC with the following condition: Column: XBridge Shield RP18 OBD Column, 19×250 mm, 10 um; Mobile Phase A: Water (0.05% TFA), Mobile Phase B: ACN; Flow rate: 25 mL/min; Gradient: 25 B to 38 B in 18 min; 220 nm; RT 1:14.25; The collected fraction was lyophilized to afford 1-(3-chloro-4-methylphenyl)-3-[[2-(2,6-dioxopiperidin-3-yl)-1-oxo-3H-isoindol-5-yl]methyl]urea (Compound 2-4, 21.4 mg, 7%) as a white solid. LCMS (ESI, ms): 441,443 [M+H]⁺

### Step 4. Synthesis of Compound 2-5

To a stirred mixture of 1-(3-chloro-4-methylphenyl)-3-[[2-(2,6-dioxopiperidin-3-yl)-1-oxo-3H-isoindol-5-yl]methyl]urea (Compound 2-5, 300.00 mg, 0.68 mmol, 1.00 equiv) and K₂CO₃ (180 mg, 1.30 mmol, 1.91 equiv) in DMF (0.50 mL), was added chloromethyl 2-methyl-2-[(5-nitropyridin-2-yl)disulfanyl]propyl carbonate (600 mg, 1.70 mmol, 2.50 equiv), TBAI (119.00 mg, 0.46 mmol, 0.67 equiv) at room temperature under nitrogen atmosphere. The resulting mixture was stirred for overnight at room temperature. LCMS indicated the reaction was completed. The residue was purified by reverse flash chromatography with the following conditions: column, C18 silica gel; mobile phase, ACN in water (0.1% FA), 00% to 85% gradient in 40 min; detector, UV 254 nm. This resulted in [3-[5-([[(3-chloro-4-methylphenyl)carbamoyl]amino]methyl)-1-oxo-3H-isoindol-2-yl]-2,6-dioxopiperidin-1-yl]methyl 2-methyl-2-[(5-nitropyridin-2-yl)disulfanyl]propyl carbonate (85 mg, 14.85%) as a brown solid. The crude product was further purified by the following condition: Column: XBridge Prep OBD C18 Column, 19×250 mm, 5 um; Mobile Phase A: Water (0.05% TFA), Mobile Phase B: ACN; Flow rate: 25 mL/min; Gradient: 53 B to 68 B in 10 min; 220 nm; RT1:9.80; The collected fraction was lyophilized to afford [3-[5-([[(3-chloro-4-methylphenyl)carbamoyl]amino]methyl)-1-oxo-3H-isoindol-2-yl]-2,6-dioxopiperidin-1-yl]methyl 2-methyl-2-[(5-nitropyridin-2-yl)disulfanyl]propyl carbonate (Compound 2-5, 8.5 mg) as white solid. LCMS (ESI): 757,757 [M+H]⁺; ¹H NMR (300 MHz, DMSO-d₆) 9.23 (d, J=2.4 Hz, 1H), 8.75 (s, 1H), 8.58 (d, J=2.7 Hz, 1H), 8.05 (d, J=8.7 Hz, 1H), 7.72-7.6 (m, 2H), 7.53 (s, 1H), 7.46 (d, J=6.3 Hz, 1H), 7.25-7.11 (m, 2H), 6.82-6.78 (m, 1H), 5.68-5.63 (m, 2H), 5.35-5.28 (m, 1H), 4.52-4.30 (m, 4H), 4.08 (s, 2H), 3.12-3.06 (m, 1H), 2.87-2.73 (m, 1H), 2.49-2.44 (m, 1H), 2.28 (s, 3H), 2.10-2.00 (m, 1H), 1.33 (s, 6H).

### Step 5. Synthesis of Compound (II)

To a stirred mixture of [3-[5-([[(3-chloro-4-methylphenyl)carbamoyl]amino]methyl)-1-oxo-3H-isoindol-2-yl]-2,6-dioxopiperidin-1-yl]methyl 2-methyl-2-[(5-nitropyridin-2-yl)disulfanyl]propyl carbonate (Compound 2-5, 70.00 mg, 0.092 mmol, 1.00 equiv) and sodium methyl sulfinate (28.00 mg, 0.27 mmol, 2.97 equiv) in DCM (14.00 mL) was added Br₂ (14 mg, 0.087 mmol, 0.95 equiv) dropwise at 0° C. The resulting mixture was stirred for 3 h at room temperature. To the above stirred mixture was added Sodium methyl sulfinate (28.00 mg, 0.27 mmol, 2.97 equiv) and Br₂ (14 mg, 0.087 mmol, 0.95 equiv) at room temperature. The resulting mixture was stirred for overnight at room temperature. LCMS indicated the reaction was completed. The resulting mixture was filtered, the filter cake was washed with DCM (20 mL). The filtrate was concentrated under reduced pressure. The residue was purified by reverse flash chromatography with the following conditions: column, C18 silica gel; mobile phase, ACN in water, 5% to 85% gradient in 40 min; detector, UV 254 nm. The crude product was purified by following condition: Column: YMC-Actus Triart C18, 30×250.5 um; Mobile Phase A: Water (0.05% TFA), Mobile Phase B: ACN; Flow rate: 25 mL/min; Gradient: 55 B to 75 B in 7 min; 220 nm; RT1:6.35. The collected fraction was lyophilized to afford 1-(3-chloro-4-methylphenyl)-3-[(2-[1-[([[2-(methanesulfonylsulfanyl)-2-methylpropoxy]carbonyl]oxy)-methyl]-2,6-dioxopiperidin-3-yl]-1-oxo-3H-isoindol-5-yl)methyl]urea (Compound (II), 3.3 mg, 5.05%) as white solid. LCMS (ESI): 681,683 [M+H]^{+ 1}H NMR (300 MHz, DMSO-d₆) 8.75 (s, 1H), 7.73-7.66 (m, 2H), 7.53 (s, 1H), 7.47 (d, J=7.8 Hz, 1H), 7.20-7.12 (m, 2H), 6.82-6.79 (m, 1H), 5.75-5.66 (m, 2H), 5.33-5.27 (m, 1H), 4.51-4.29 (m, 6H), 3.35 (s, 3H), 3.11-3.06 (m, 1H), 2.87-2.73 (m, 1H), 2.49-2.44 (m, 1H), 2.28 (s, 3H), 2.10-2.00 (m, 1H), 1.48 (s, 6H)

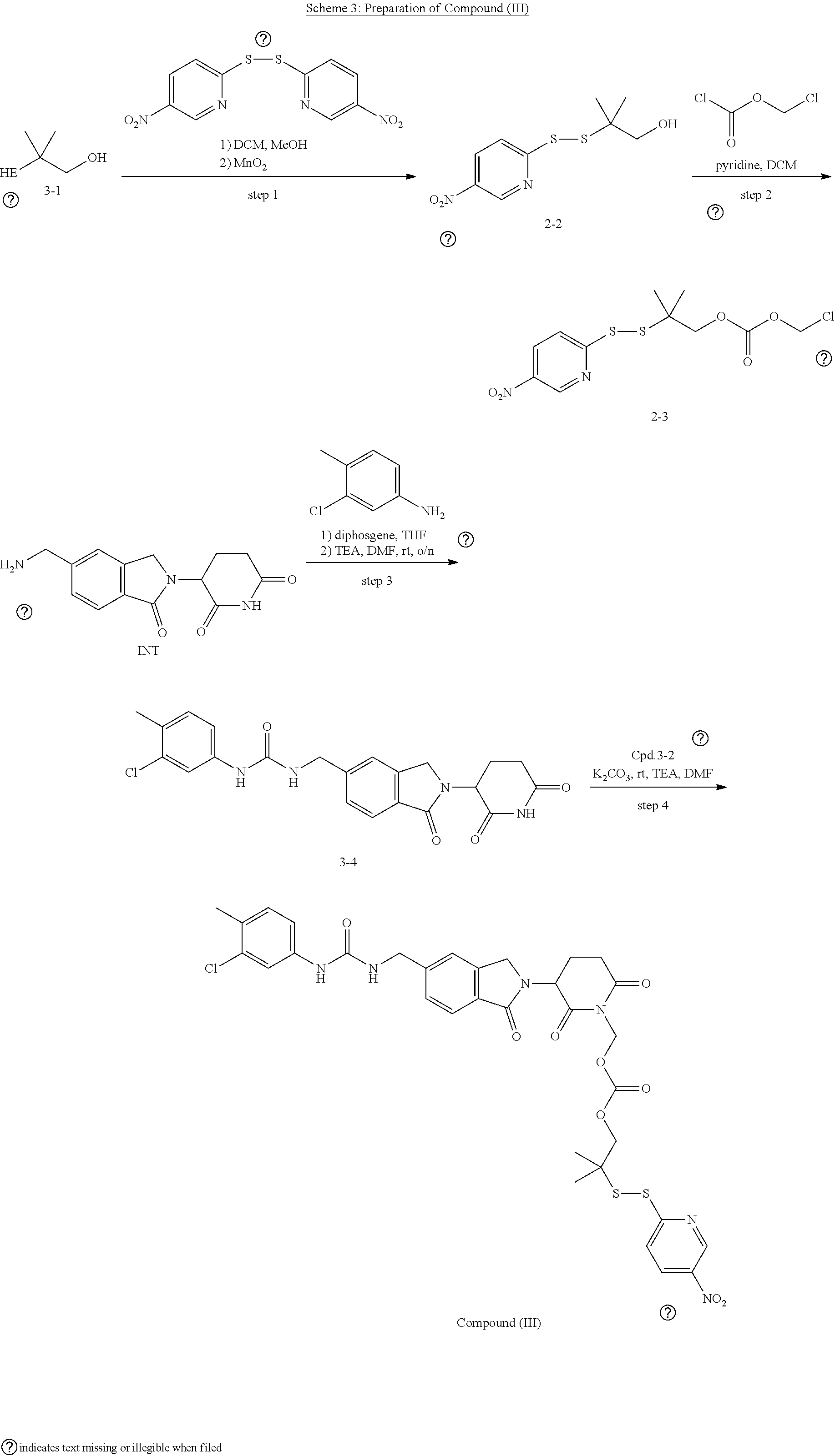

### Step 1. Synthesis of Compound 3-2

To a stirred mixture of 2-methyl-2-sulfanylpropan-1-ol (1.00 g, 9.41 mmol, 1.00 equiv) in DCM (3.00 mL) and MeOH (3.00 mL) was added 5-nitro-2-[(5-nitropyridin-2-yl)disulfanyl]pyridine (1.46 g, 4.70 mmol, 0.50 equiv) at room temperature. The resulting mixture was stirred for overnight at room temperature. To the above mixture was added MnO2 (1.50 g, 17.25 mmol, 1.83 equiv) in portions over at room temperature. The resulting mixture was stirred for additional 2 h at room temperature. LCMS indicated the reaction was completed. The resulting mixture was filtered, the filter cake was washed with DCM (20 mL). The filtrate was concentrated under reduced pressure. The residue was purified by silica gel column chromatography, eluted with PE/EtOAc (8:1) to afford 2-methyl-2-[(5-nitropyridin-2-yl)disulfanyl]propan-1-ol (Compound 3-2, 1.4 g, 57%) as an orange solid. LCMS (ESI, ms): 261 [M+H].

### Step 2. Synthesis of Compound 3-3

To a stirred mixture of 2-methyl-2-[(5-nitropyridin-2-yl)disulfanyl]propan-1-ol (Compound 3-2, 1.20 g, 4.61 mmol, 1.00 equiv) and pyridine (0.90 g, 11.38 mmol, 2.47 equiv) in DCM (20.00 mL) were added chloromethyl chloroformate (0.60 g, 4.65 mmol, 1.01 equiv) in DCM (2.00 mL) dropwise at 0° C. The resulting mixture was stirred overnight at room temperature. LCMS detected 30% desired product. The reaction was quenched with water/ice. The resulting mixture was extracted with DCM (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (10:1) to afford chloromethyl 2-methyl-2-[(5-nitropyridin-2-yl)disulfanyl]propyl carbonate (Compound 3-3, 330 mg, 20%) as alight yellow oil. LCMS (ESI, ms): 353,355 [M+H]⁺.

### Step 3. Synthesis of Compound 3-4

To a stirred mixture of 3-chloro-p-toluidine (102 mg, 0.72 mmol, 0.99 equiv) in THE (10.00 mL) was added diphosgene (145.00 mg, 0.73 mmol, 1.00 equiv) dropwise at 0° C. under nitrogen atmosphere. The resulting mixture was stirred for 1 h at room temperature under nitrogen atmosphere. The resulting mixture was concentrated under reduced pressure. To a stirred mixture of 3-[5-(aminomethyl)-1-oxo-3H-isoindol-2-yl]piperidine-2,6-dione (INT, prepared according to the procedure described for Compound 1-4, 200 mg, 0.73 mmol, 1.00 equiv) and TEA (61.00 mg, 0.60 mmol, 0.82 equiv) in DMF (10.00 mL) were added the above mixture in DMF (15.00 mL) dropwise at 0 degrees C. under nitrogen atmosphere. The resulting mixture was stirred for 3 h at room temperature under nitrogen atmosphere. LCMS indicated the reaction was completed. The residue was purified by reverse flash chromatography with the following conditions: column, C18 silica gel; mobile phase, ACN in water (0.1% FA), 0% to 80% gradient in 40 min; detector, UV 254 nm. This resulted in 1-(3-chloro-4-methylphenyl)-3-[[2-(2,6-dioxopiperidin-3-yl)-1-oxo-3H-isoindol-5-yl]methyl]urea (Compound 3-4, 85 mg, 26.34%) as a white solid. The product was purified by Prep-HPLC with the following condition: Column: XBridge Shield RP18 OBD Column, 19×250 mm, 10 um; Mobile Phase A: Water (0.05% TFA), Mobile Phase B: ACN; Flow rate: 25 mL/min; Gradient: 25 B to 38 B in 18 min; 220 nm; RT 1: 14.25; The collected fraction was lyophilized to afford 1-(3-chloro-4-methylphenyl)-3-[[2-(2,6-dioxopiperidin-3-yl)-1-oxo-3H-isoindol-5-yl]methyl]urea (Compound 3-4, 21.4 mg, 7%) as a white solid. LCMS (ESI, ms): 441,443 [M+H]⁺

### Step 4. Synthesis of Compound (III)

To a stirred mixture of 1-(3-chloro-4-methylphenyl)-3-[[2-(2,6-dioxopiperidin-3-yl)-1-oxo-3H-isoindol-5-yl]methyl]urea (Compound 3-4, 300.00 mg, 0.68 mmol, 1.00 equiv) and K₂CO₃ (180 mg, 1.30 mmol, 1.91 equiv) in DMF (0.50 mL), was added chloromethyl 2-methyl-2-[(5-nitropyridin-2-yl)disulfanyl]propyl carbonate (Compound 3-3, 600 mg, 1.70 mmol, 2.50 equiv), TBAI (119.00 mg, 0.46 mmol, 0.67 equiv) at room temperature under nitrogen atmosphere. The resulting mixture was stirred for overnight at room temperature. LCMS indicated the reaction was completed. The residue was purified by reverse flash chromatography with the following conditions: column, C18 silica gel; mobile phase, ACN in water (0.1% FA), 0% to 85% gradient in 40 min; detector, UV 254 nm. This resulted in [3-[5-([[(3-chloro-4-methylphenyl)carbamoyl]amino]methyl)-1-oxo-3H-isoindol-2-yl]-2,6-dioxopiperidin-1-yl]methyl 2-methyl-2-[(5-nitropyridin-2-yl)disulfanyl]propyl carbonate (Compound (III), 85 mg, 14.85%) as a brown solid. The crude product was further purified by the following condition: Column: XBridge Prep OBD C18 Column, 19×250 mm, 5 um; Mobile Phase A: Water (0.05% TFA), Mobile Phase B: ACN; Flow rate: 25 mL/min; Gradient: 53 B to 68 B in 10 min; 220 nm; RT1:9.80; The collected fraction was lyophilized to afford [3-[5-([[(3-chloro-4-methylphenyl)carbamoyl]amino]methyl)-1-oxo-3H-isoindol-2-yl]-2,6-dioxopiperidin-1-yl]methyl 2-methyl-2-[(5-nitropyridin-2-yl)disulfanyl]propyl carbonate (8.5 mg) as white solid. LCMS (ESI): 757,757 [M+H]⁺; ¹H NMR (300 MHz, DMSO-d₆) 9.23 (d, J=2.4 Hz, 1H), 8.75 (s, 1H), 8.58 (d, J=2.7 Hz, 1H), 8.05 (d, J=8.7 Hz, 1H), 7.72-7.6 (m, 2H), 7.53 (s, 1H), 7.46 (d, J=6.3 Hz, 1H), 7.25-7.11 (m, 2H), 6.82-6.78 (m, 1H), 5.68-5.63 (m, 2H), 5.35-5.28 (m, 1H), 4.52-4.30 (m, 4H), 4.08 (s, 2H), 3.12-3.06 (m, 1H), 2.87-2.73 (m, 1H), 2.49-2.44 (m, 1H), 2.28 (s, 3H), 2.10-2.00 (m, 1H), 1.33 (s, 6H).

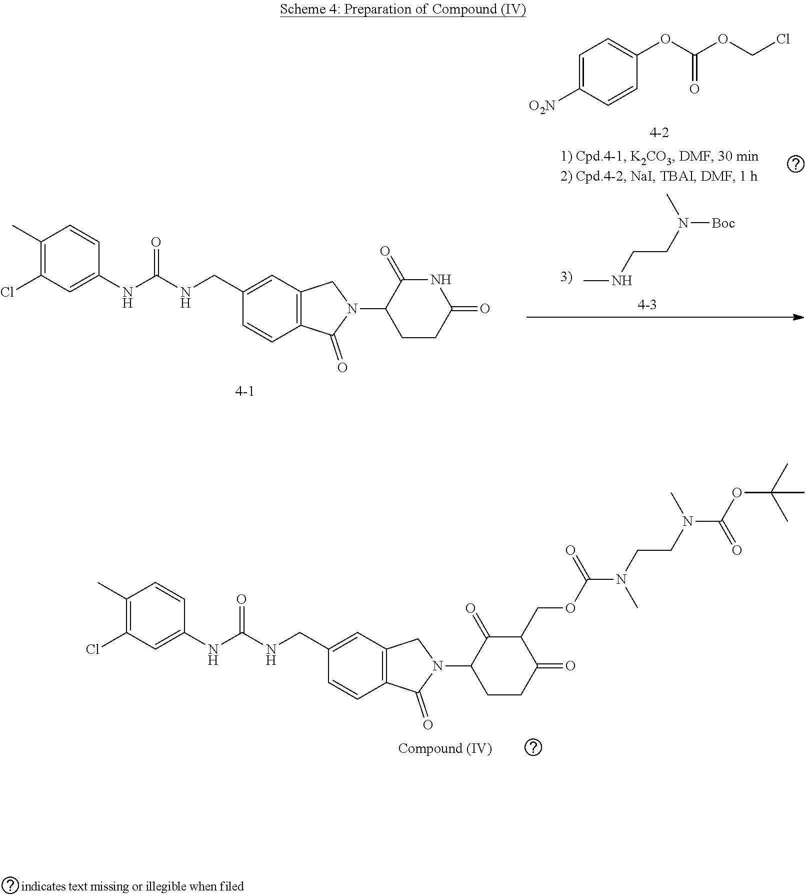 [SMILES: O=C(OCCl)Oc1ccc([N+](=O)[O-])cc1 ; Cc1ccc(NC(=O)NCc2ccc3c(c2)CN(C2CCC(=O)NC2=O)C3=O)cc1Cl ; CNCCN(C)C(=O)OC(C)(C)C ; Cc1ccc(NC(=O)NCc2ccc3c(c2)CN(C2CCC(=O)C(COC(=O)N(C)CCN(C)C(=O)OC(C)(C)C)C2=O)C3=O)cc1Cl]

### Step 1. Synthesis of Compound (IV)

To a stirred mixture of 1-(3-chloro-4-methylphenyl)-3-[[2-(2,6-dioxopiperidin-3-yl)-1-oxo-3H-isoindol-5-yl]methyl]urea (Compound 4-1, prepared according to the procedure described for Compound 1-5, 200.00 mg, 0.45 mmol, 1.00 equiv) in DMF (2.00 mL) was added K₂CO₃ (200.00 mg, 1.44 mmol, 3.19 equiv) in portions at 0° C. in dark. The resulting mixture was stirred for 1 h at 0° C. To the above mixture was added NaI (80 mg, 0.53 mmol, 1.18 equiv), TBAI (40 mg, 0.10 mmol, 0.24 equiv) and chloromethyl 4-nitrophenyl carbonate (Compound 4-2, 320 mg, 1.38 mmol, 3.05 equiv) at 0° C. in darkness. The resulting mixture was stirred for additional 1 h at 0° C. To the above mixture was added tert-butyl N-methyl-N-[2-(methylamino)ethyl]carbamate (Compound 4-3, 180 mg, 0.95 mmol, 2.11 equiv) in DMF (0.10 mL) dropwise at 0 degrees C. in dark. The resulting mixture was stirred for additional 3 h at 0° C. in darkness. 24% of desired product could be detected by LCMS. The reaction mixture was purified by the following condition: Column: Kinetex EVO C18 Column, 30×150, Sum; Mobile Phase A: Water (0.05% TFA), Mobile Phase B: ACN; Flow rate: 25 mL/min; Gradient: 20 B to 45 B in 14 min, 210 nm; RT1:12.93 min. The collected fraction was lyophilized to afford [3-[5-([[(3-chloro-4-methylphenyl)carbamoyl]-amino]methyl)-1-oxo-3H-isoindol-2-yl]-2,6-dioxopiperidin-1-yl]methyl N-[2-[(tert-butoxycarbonyl)(methyl)amino]ethyl]-N-methylcarbamate (Compound (IV), 23.9 mg, 7%) as a white solid. LCMS: (ms, ESI): 707, 709 [M+Na]⁺, 585, 587 [M+H−100]^{+ 1}HNMR: (400 MHz, DMSO-d₆): 8.81 (s, 1H), 7.71-7.66 (m, 2H), 7.51 (s, 1H), 7.45 (d, J=8.0 Hz, 1H), 7.18-7.11 (m, 2H), 6.86 (t, J=6.0 Hz, 1H), 5.61-5.56 (m, 2H), 5.27-5.24 (m, 1H), 4.49-4.26 (m, 4H), 3.29 (s, 3H), 3.21 (s, 1H), 3.08-3.03 (m, 1H), 2.83-2.66 (m, 7H), 2.42-2.38 (m, 1H), 2.22 (s, 3H), 2.07-2.05 (m, 1H), 1.35 (s, 9H).

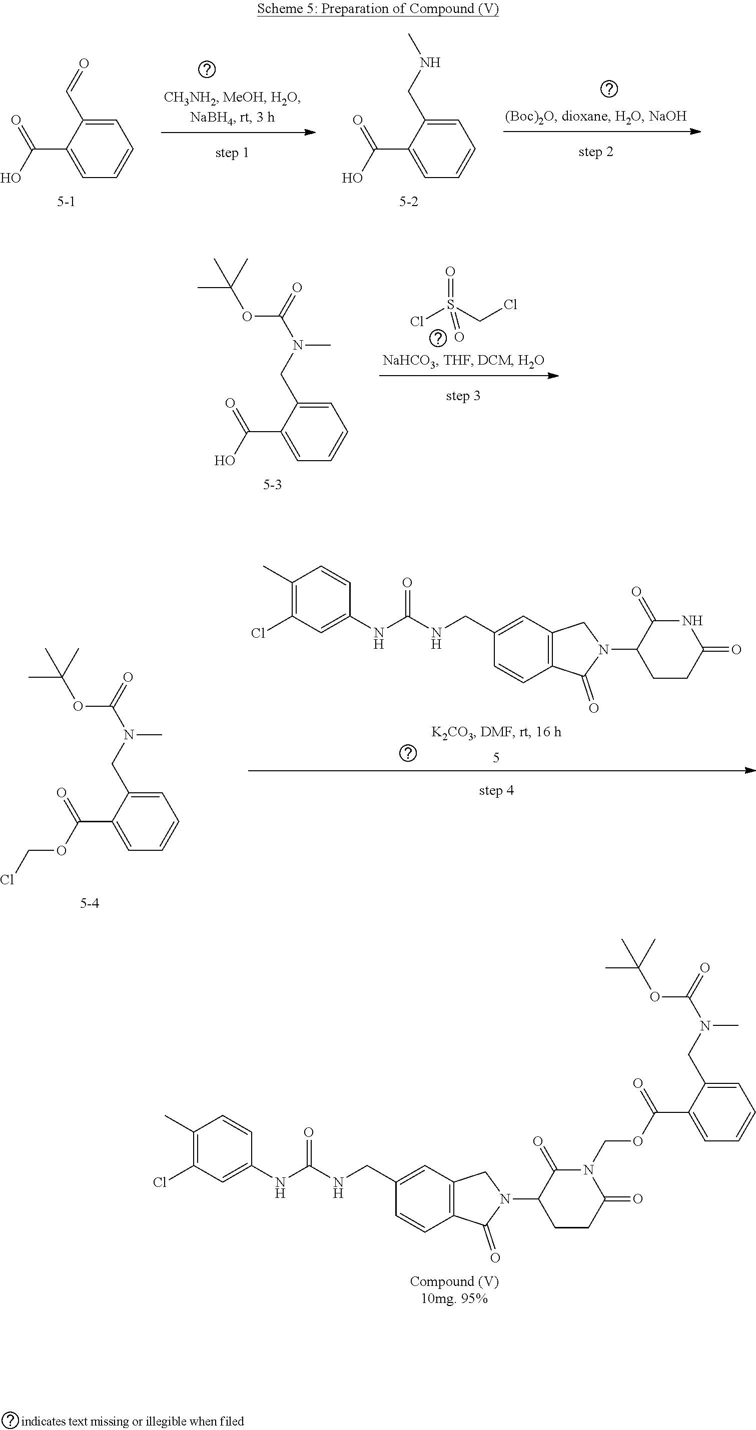

### Step 1. Synthesis of Compound 5-2

To a stirred mixture of 2-carboxybenzaldehyde (Compound 5-1, 2.00 g, 12.65 mmol, 1.00 equiv) in MeOH (27.00 mL) was added CH₃NH₂·HCl (0.79 g, 25.30 mmol, 2.00 equiv) in H₂O (4.00 mL) at 0° C. The resulting mixture was stirred for 1 h at 25° C. To the above mixture was added NaBH₄ (0.24 g, 6.33 mmol, 0.50 equiv) at 25° C. The resulting mixture was stirred for additional 0.5 h at 25° C. LCMS indicated the reaction was completed. The reaction was quenched by the addition of acetone (20 mL) at room temperature. The resulting mixture was concentrated under reduced pressure. The residue was purified by trituration with acetone (30 mL). This resulted in 2-[(methylamino)methyl]benzoic acid (Compound 5-2, 2 g, 86%) as a white solid. LCMS (ES, m/z). 166 [M+H]⁺

### Step 2. Synthesis of Compound 5-3

To a stirred mixture of 2-[(methylamino)methyl]benzoic acid (Compound 5-2, 1.00 g, 5.44 mmol, 1.00 equiv), NaOH in H₂O (1 M) (20.00 mL) in dioxane (27.00 mL) was added (BOC)₂₀ (2.38 g, 10.88 mmol, 2.00 equiv) at 0° C. The resulting mixture was stirred for 2 h at 25° C. LCMS indicated the reaction was completed. The resulting mixture was concentrated under reduced pressure. The residue was acidified to pH 3 with HCl (1N, aq.). The resulting mixture was extracted with EtOAc (3×30 mL). The combined organic layers were washed with brine (30 mL), dried over anhydrous Na₂SO₄. After filtration, the filtrate was concentrated under reduced pressure. The crude product was used to next step without further purification. LCMS (ES, m/z): 266 [M+H]⁺

### Step 3. Synthesis of Compound 5-4

To a stirred mixture of 2-([[(tert-butoxy)carbonyl](methyl)amino]methyl)benzoic acid (Compound 5-3, 500 mg, 1.70 mol, 1 equiv) in DCM (6 mL) and H₂O (7.5 mL) was added NaHCO₃ (570 mg, 6.78 mmol, 4 equiv) and tetrabutylammonium hydrogen sulfate (57 mg, 0.17 mmol, 0.10 equiv) at 0° C. The resulting mixture was stirred for 10 min at 0° C. To the above mixture was added chloromethanesulfonyl chloride (303 mg, 2.04 mol, 1.20 equiv) at 0° C. The resulting mixture was stirred for additional 3 h at 25° C. LCMS indicated the reaction was completed. The reaction was quenched by the addition of water (20 mL) at room temperature. The resulting mixture was extracted with CH₂Cl₂ (3×30 mL). The combined organic layers were washed with brine (21 mL), dried over anhydrous Na₂SO₄. After filtration, the filtrate was concentrated under reduced pressure. This resulted in chloromethyl 2-([[(tert-butoxy)carbonyl](methyl)amino]methyl)benzoate (Compound 5-4, 250 mg, 42%) as a yellow oil. LCMS (ES, m/z): 314,316 [M+H]⁺, 214, 216 [M+H−100]⁺

### Step 4. Synthesis of Compound (V)

To a stirred mixture of 1-(3-chloro-4-methylphenyl)-3-[[2-(2,6-dioxopiperidin-3-yl)-1-oxo-2,3-dihydro-1H-isoindol-5-yl]methyl]urea (Compound 5, prepared according to the procedure described for Compound 1-5, 100 mg, 0.20 mmol, 1.00 equiv) and K₂CO₃ (84 mg, 0.61 mmol, 3.00 equiv) in DMF (2.00 mL) was added chloromethyl 2-([[(tert-butoxy)carbonyl](methyl)amino]methyl)benzoate (Compound 5-4, 128 mg, 0.40 mmol, 2 equiv) at 0° C. The resulting mixture was stirred for 16 h at 25° C. LCMS indicated the reaction was completed. The reaction was quenched by the addition of water (10 mL) at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EtOAc (1:1). The crude product was purified by Prep-HPLC with the following conditions Column, XSelect CSH Fluoro Phenyl, 30 mm×150 mm, 5 um; mobile phase, water (0.1% FA) and ACN (45% to 58% in 10 min); Detector, UV 254 nm. The collected fraction was lyophilized to afford [3-[5-([[(3-chloro-4-methylphenyl)carbamoyl]amino]methyl)-1-oxo-2,3-dihydro-1H-isoindol-2-yl]-2,6-dioxopiperidin-1-yl]methyl 2-([[(tert-butoxy)carbonyl](methyl)amino]methyl)benzoate (Compound (V), 9.4 mg, 6%) as a white solid. LCMS (ES, m/z): 716, 718 [M−H]^{- 1}H-NMR (DMSO-d₆, 400 MHz) δ (ppm): 8.83 (br s, 1H), 7.83-7.80 (m, 1H), 7.71-7.61 (m, 3H), 7.51-7.38 (m, 3H), 7.19-7.11 (m, 3H), 6.90 (br s, 1H), 5.93-5.82 (m, 2H), 5.35-5.30 (m, 1H), 4.67 (d, J=6.8 Hz, 2H), 4.46-4.29 (m, 4H), 3.20-3.05 (m, 1H), 2.96-2.86 (m, 4H), 2.44-2.43 (m, 1H), 2.22 (s, 3H), 2.08-2.06 (m, 1H), 1.43-1.26 (m, 9H).

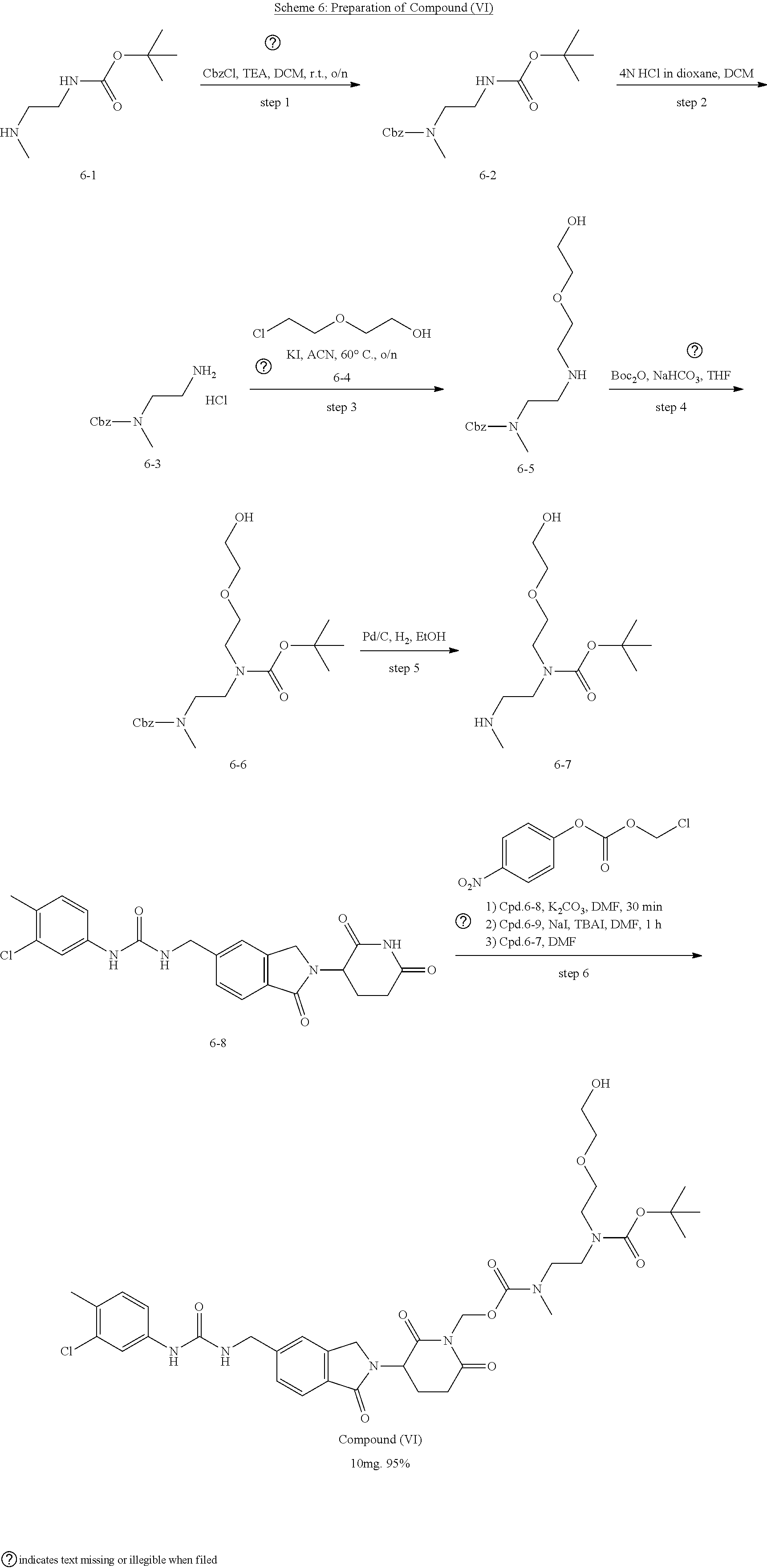

### Step 1. Synthesis of Compound 6-2

To a stirred mixture of tert-butyl N-[2-(methylamino)ethyl]carbamate (Compound 6-1, 2.00 g, 11.48 mmol, 1.00 equiv) and TEA (1.40 g, 13.83 mmol, 1.21 equiv) in DCM (20.00 mL) was added CbzCl (2.05 g, 12.01 mmol, 1.05 equiv) in DCM (5 mL) dropwise at 0° C. The resulting mixture was stirred for 1 h at room temperature. LCMS showed the reaction was completed. The reaction was quenched by the addition of Water. The resulting mixture was extracted with CH₂Cl₂ (3×20 mL). The combined organic layers were washed with brine (20 mL), dried over anhydrous Na₂SO₄. After filtration, the filtrate was concentrated under reduced pressure. This resulted in tert-butyl N-(2-[[(benzyloxy)carbonyl](methyl)amino]ethyl)carbamate (Compound 6-2, 3.5 g, 98%) as a light yellow oil. LCMS (ms, ESI): 309 [M+H]⁺, 331 [M+Na]⁺

### Step 2. Synthesis of Compound 6-3

To a stirred solution of tert-butyl N-(2-[[(benzyloxy)carbonyl]-(methyl)amino]ethyl)carbamate (compound 6-2, 1.50 g) in DCM (20.00 mL) was added HCl (4N) in 1,4-dioxane (20.00 mL) dropwise at 0° C. under nitrogen atmosphere. The resulting mixture was stirred for 1 h at room temperature. LCMS showed the reaction was completed. The resulting mixture was concentrated under reduced pressure to afford benzyl N-(2-aminoethyl)-N-methylcarbamate hydrochloride (Compound 6-3, 1.4 g, crude) as a white solid. LCMS (ESI, ms): 209 [M+H]⁺

### Step 3. Synthesis of Compound 6-5

To a stirred solution of benzyl N-(2-aminoethyl)-N-methylcarbamate hydrochloride (Compound 6-3, 1.20 g, 4.90 mmol, 1.00 equiv) and K₂CO₃ (2.03 g, 14.71 mmol, 3.00 equiv) in ACN (150 mL) was added KI (0.41 g, 2.45 mmol, 0.50 equiv) and ethanol, 2-(2-chloroethoxy)-(0.73 g, 5.88 mmol, 1.20 equiv) at room temperature under nitrogen atmosphere. The resulting mixture was stirred for overnight at 60 degrees C. under nitrogen atmosphere. LCMS indicated the reaction was completed. The resulting mixture was filtered, the filter cake was washed with MeCN (3×100 mL). The filtrate was concentrated under reduced pressure. The resulting mixture was used in the next step directly without further purification. LCMS (ESI, ms): 297 [M+H]⁺

### Step 4. Synthesis of Compound 6-6

To a stirred solution of benzyl N-(2-[[2-(2-hydroxyethoxy)ethyl]amino]ethyl)-N-methylcarbamate (Compound 6-5, 1.40 g, 4.72 mmol, 1.00 equiv) and NaHCO₃ (396 mg, 4.72 mmol, 1.00 equiv) in THE (14.00 mL) and H₂O (14.00 mL) was added Boc₂O (1.03 g, 4.72 mmol, 1.00 equiv) at 0° C. under nitrogen atmosphere. The resulting mixture was stirred for overnight at room temperature under nitrogen atmosphere. LCMS indicated complete reaction The resulting mixture was extracted with EtOAc (3×20 mL). The combined organic layers were washed with brine (3×20 mL), dried over anhydrous Na₂SO₄. After filtration, the filtrate was concentrated under reduced pressure to afford benzyl N-[2-[(tert-butoxycarbonyl)[2-(2-hydroxyethoxy)ethyl]amino]ethyl]-N-methylcarbamate (Compound 6-6, 1.4 g, 74%) as a white solid. LCMS (ESI, ms): 397 [M+H]^{+ 1}H NMR (300 MHz, Chloroform-d) δ 7.42-7.29 (m, 5H), 5.13 (s, 2H), 3.81-3.31 (m, 12H), 2.97 (t, J=2.7 Hz, 3H), 1.46 (s, 9H).

### Step 5. Synthesis of Compound 6-7

To a stirred solution of benzyl N-[2-[(tert-butoxycarbonyl)[2-(2-hydroxyethoxy)ethyl]amino]ethyl]-N-methylcarbamate (Compound 6-6, 1.30 g, 3.28 mmol, 1.00 equiv) in EtOH (65.00 mL) was added Pd/C (26 mg, 10%) at room temperature. The resulting mixture was stirred for overnight at room temperature under H₂ atmosphere. LCMS indicated complete reaction. The resulting mixture was filtered, the filter cake was washed with EtOH (3×10 mL). The filtrate was concentrated under reduced pressure to afford tert-butyl N-[2-(2-hydroxyethoxy)ethyl]-N-[2-(methylamino)ethyl]carbamate (800 mg, 93%) as an off-white solid. ¹H NMR (300 MHz, Chloroform-d) δ 3.70-3.64 (m, 2H), 3.59 (d, J=5.7 Hz, 2H), 3.55-3.50 (m, 2H), 3.39 (s, 4H), 3.11 (s, 1H), 2.77 (t, J=6.3 Hz, 2H), 2.41 (s, 3H), 1.44 (s, 9H).

### Step 6. Synthesis of Compound (VI)

To a stirred solution of 1-(3-chloro-4-methylphenyl)-3-[[2-(2,6-dioxopiperidin-3-yl)-1-oxo-3H-isoindol-5-yl]methyl]urea (Compound 6-7, 200 mg, 0.45 mmol, 1.00 equiv) in DMF (2.00 mL) was added K₂CO₃ (188 mg, 1.36 mmol, 3.00 equiv) at room temperature under nitrogen atmosphere. The resulting mixture was stirred for 30 min at room temperature under nitrogen atmosphere. To the above mixture was added chloromethyl 4-nitrophenyl carbonate (105 mg, 0.45 mmol, 1.00 equiv), NaI (34 mg, 0.22 mmol, 0.50 equiv) and TBAI (167 mg, 0.45 mmol, 1.00 equiv) at room temperature. The resulting mixture was stirred for additional 1 h at room temperature. Then tert-butyl N-[2-(2-hydroxyethoxy)ethyl]-N-[2-(methylamino)ethyl]carbamate (238 mg, 0.90 mmol, 2.00 equiv) was added, The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 80% gradient in 40 min; detector, UV 254 nm. The collected fraction was lyophilized. This resulted in [3-[5-([[(3-chloro-4-methylphenyl)carbamoyl]amino]methyl)-1-oxo-3H-isoindol-2-yl]-2,6-dioxopiperidin-1-yl]methyl N-[2-[(tert-butoxycarbonyl)[2-(2-hydroxyethoxy)ethyl]amino]ethyl]-N-methylcarbamate (Compound (VI), 50 mg, 14.52%) as a white solid. The crude product (50 mg) was purified by Prep-HPLC with the following conditions: Column, XSelect CSH Fluoro Phenyl, 30 mm×150 mm, 5 um; mobile phase, Water (0.05% FA) and ACN (43% PhaseB up to 63% in 7 min); Detector, UV 254 nm. The collected fraction was lyophilized to afford [3-[5-([[(3-chloro-4-methylphenyl)carbamoyl]amino]methyl)-1-oxo-3H-isoindol-2-yl]-2,6-dioxopiperidin-1-yl]methyl N-[2-[(tert-butoxycarbonyl)[2-(2-hydroxyethoxy)-ethyl]amino]ethyl]-N-methylcarbamate (13.3 mg, 3.86%) as a white solid. LCMS (ESI, ms): 759,761 [M+H]⁺, 559, 561 [M+H−100]^{+ 1}H NMR (400 MHz, DMSO-d₆) δ 8.75 (s, 1H), 7.75-7.60 (m, 2H), 7.54-7.41 (m, 2H), 7.23-7.10 (m, 2H), 6.80 (t, J=6.0 Hz, 1H), 5.64-5.47 (m, 2H), 5.26-5.22 (m, 2H), 4.56 (br s, 1H), 4.50-4.28 (m, 4H), 3.46 (d, J=5.2 Hz, 4H), 3.42-3.41 (m, 2H), 3.29-3.28 (m, 2H), 3.28-3.17 (m, 4H), 3.07-3.05 (m, 1H), 2.87-2.76 (m, 4H), 2.49-2.47 (m, 1H), 2.23 (s, 3H), 2.07 (s, 1H), 1.36 (s, 9H).

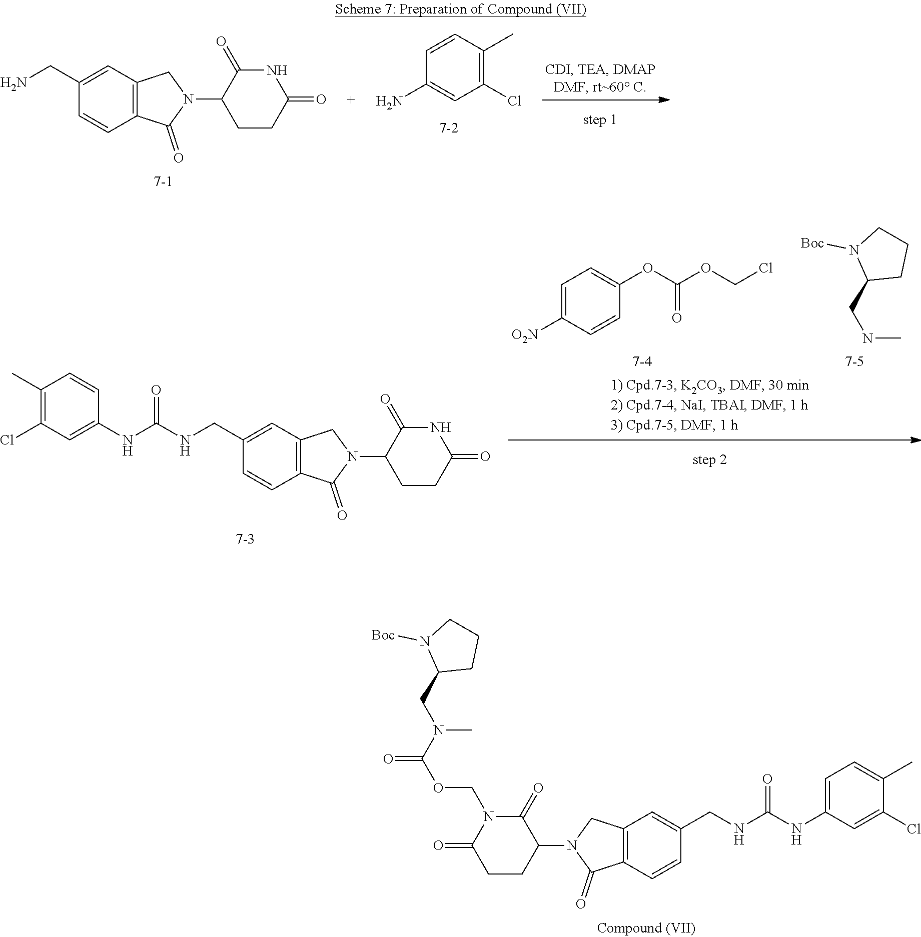

### Step 1 Synthesis of Compound 7-3

To a stirred solution of 3-[5-(aminomethyl)-1-oxo-3H-isoindol-2-yl]piperidine-2,6-dione (Compound 7-1, prepared according to the procedure described for Compound 1-4, 1.00 g, 3.66 mmol, 1.00 equiv) and TEA (0.37 g, 3.66 mmol, 1.00 equiv) in DMF (10 mL) was added CDI (0.59 g, 3.66 mmol, 1.00 equiv) at 0° C. under nitrogen atmosphere. The resulting mixture was stirred for 2 h at room temperature under nitrogen atmosphere. To the above mixture was added DMAP (1.34 g, 10.98 mmol, 3.00 equiv) and 3-chloro-p-toluidine (0.52 g, 3.66 mmol, 1.00 equiv) at room temperature. The resulting mixture was stirred for additional overnight at 60 degrees C. LCMS indicated the reaction was completed. The mixture was allowed to cool down to room temperature. The reaction mixture was poured into ice/water, Then the resulting mixture was filtered, the filter cake was washed with MeCN (3×50 mL). The filtered cake was dried under infrared light. This resulted in 1-(3-chloro-4-methylphenyl)-3-[[2-(2,6-dioxopiperidin-3-yl)-1-oxo-3H-isoindol-5-yl]methyl]urea (Compound 7-3, 1.1 g, 68%) as a white solid. LCMS (ESI, ms): 441,443 [M+H]⁺.

### Step 2. Synthesis of Compound (VII)

To a stirred solution of 1-(3-chloro-4-methylphenyl)-3-[[2-(2,6-dioxopiperidin-3-yl)-1-oxo-3H-isoindol-5-yl]methyl]urea (Compound 7-3, 200.00 mg, 0.45 mmol, 1.00 equiv) in DMF (2.00 mL) was added K₂CO₃ (188 mg, 1.36 mmol, 3.00 equiv) at room temperature under nitrogen atmosphere. The resulting mixture was stirred for 30 min at room temperature under nitrogen atmosphere. To the above mixture was added chloromethyl 4-nitrophenyl carbonate (Compound 7-4, 315 mg, 1.36 mmol, 3.00 equiv), TBAI (84 mg, 0.23 mmol, 0.50 equiv) and NaI (68 mg, 0.45 mmol, 1.00 equiv) at room temperature. The resulting mixture was stirred for additional 1 h at room temperature. Then tert-butyl (2S)-2-[(methylamino)methyl]pyrrolidine-1-carboxylate (Compound 7-5, 194 mg, 0.91 mmol, 2.00 equiv) was added. The final reaction mixture was stirred for 1 h at room temperature. LCMS indicated the reaction was completed. The reaction mixture was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford tert-butyl (2S)-2-([[([3-[5-([[(3-chloro-4-methylphenyl)carbamoyl]amino]methyl)-1-oxo-3H-isoindol-2-yl]-2,6-dioxopiperidin-1-yl]methoxy)carbonyl](methyl)amino]methyl)pyrrolidine-1-carboxylate (Compound (VII), 5 mg, 2%) as a white solid. LCMS (ESI, ms): 711, 713 [M+H]⁺, 611, 613 [M+H−100]⁺. ¹H NMR (400 MHz, DMSO-d₆) δ 8.75 (s, 1H), 7.71-7.60 (m, 2H), 7.52-7.46 (m, 2H), 7.19-7.13 (m, 2H), 6.80 (s, 1H), 5.61-5.47 (m, 2H), 5.32-5.18 (m, 1H), 4.60 (s, 1H), 4.52-4.26 (m, 4H), 3.46-3.40 (m, 4H), 3.39 (s, 1H), 3.30-3.22 (m, 4H), 3.14-3.08 (m, 1H), 2.91-2.78 (m, 4H), 2.33 (s, 1H), 2.22 (s, 3H), 2.07 (s, 1H), 1.36 (s, 9H).

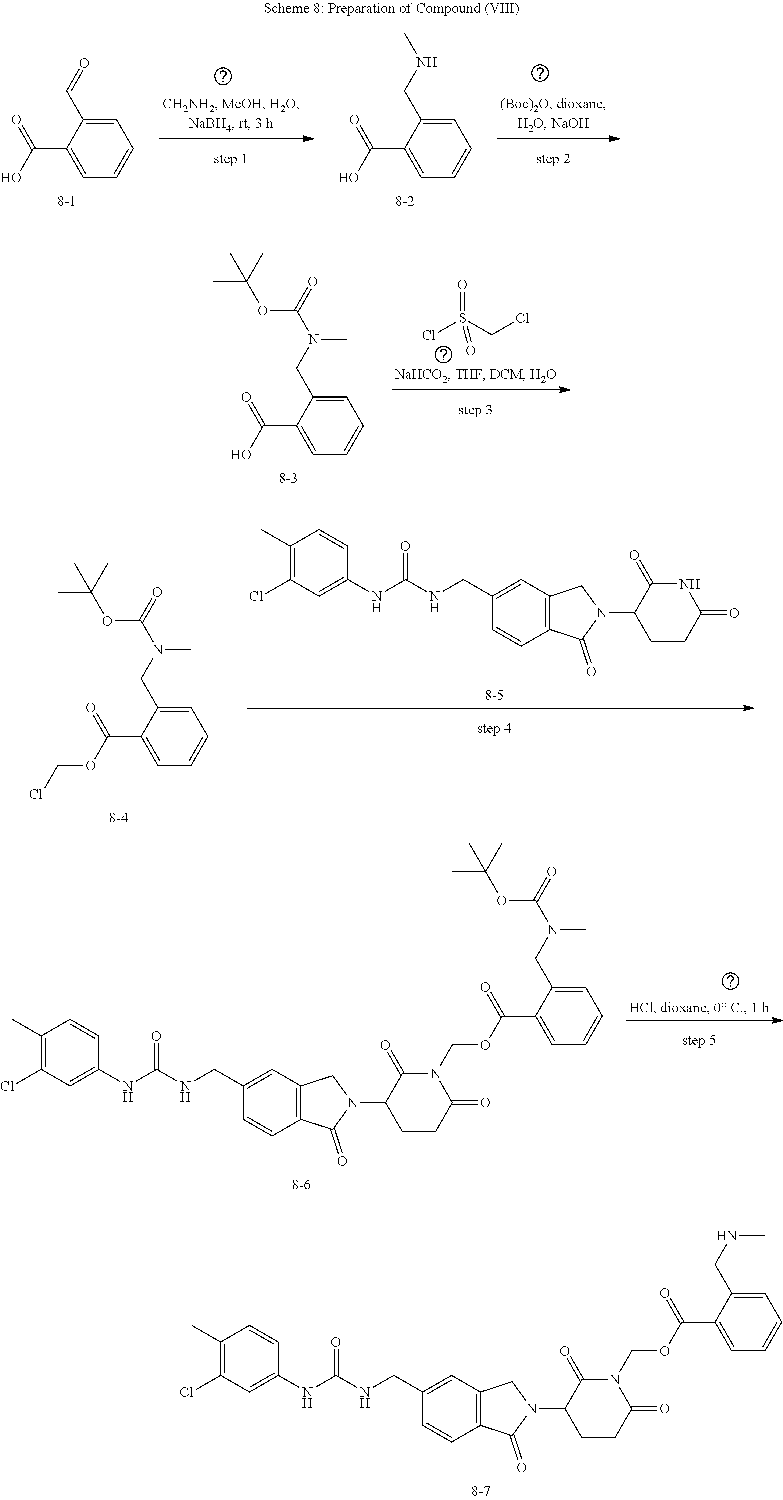

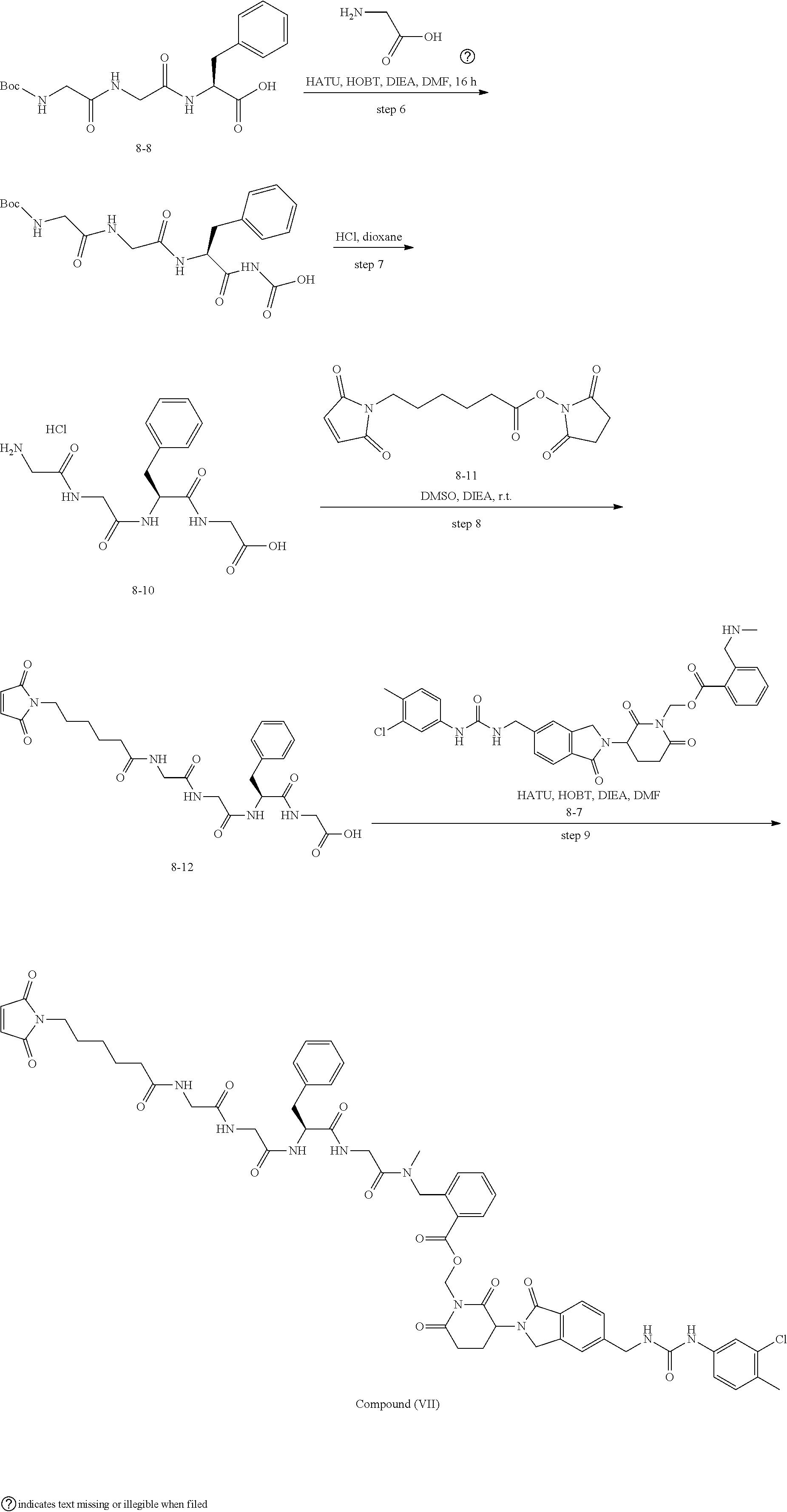

### Step 1. Synthesis of Compound 8-2

To a stirred mixture of 2-carboxybenzaldehyde (Compound 8-1, 10 g, 63.27 mmol, 1.00 equiv) in McOH (100 mL) was added CH₃NH₂ (65.0 mL, 129.72 mmol, 2N in THF, 2.05 equiv) in H₂O (20 mL) at 0° C. The resulting mixture was stirred for 1 h at 25° C. To the above mixture was added NaBH₄ (1.20 g, 31.72 mmol, 0.50 equiv) at 25° C. The resulting mixture was stirred for additional 2 h at 25 degrees C. LCMS indicated the reaction was completed. The reaction was quenched by the addition of Acetone (100 mL) at room temperature. The resulting mixture was concentrated under reduced pressure. The residue was purified by trituration with acetone (100 mL). This resulted in 2-[(methylamino)methyl]benzoic acid (Compound 8-2, 10.4 g, 84%) as an off-white solid. LCMS (ES, m/z): 166 [M+H]⁺.

### Step 2. Synthesis of Compound 8-3

To a stirred mixture of 2-[(methylamino)methyl]benzoic acid (Compound 8-2, 9 g, 54.48 mmol, 1.00 equiv) in dioxane (90 mL) was added NaOH in H₂O (1 M) (90 mL) and (Boc)₂₀ (24 g, 108.96 mmol, 2.00 equiv) at 0° C. The resulting mixture was stirred for 4 h at 25° C. LCMS indicated the reaction was completed. The residue was acidified to pH 3 with HCl (aq.). The resulting mixture was extracted with CH₂Cl₂ (3×300 mL). The combined organic layers were washed with brine (30 mL), dried over anhydrous Na₂SO₄. After filtration, the filtrate was concentrated under reduced pressure to afford 2-[[(tert-butoxycarbonyl)(methyl)amino]methyl]benzoic acid (Compound 8-3, 12 g, 81%) as a yellow oil. LCMS (ES, m/z): 266 [M+H]⁺, 166 [M+H−100]⁺.

### Step 3. Synthesis of Compound 8-4

To a stirred mixture of 2-([[(tert-butoxy)carbonyl](methyl)amino]methyl)benzoic acid (Compound 8-3, 8 g, 27.14 mmol, 1.00 equiv) in DCM (80 mL) and H₂O (80 mL) was added NaHCO₃ (9 g, 108.55 mmol, 4.00 equiv), Tetrabutylammonium hydrogen sulfate (0.92 g, 2.71 mmol, 0.10 equiv) and chloromethanesulfonyl chloride (4.9 g, 32.82 mmol, 1.2 equiv) at 0 degrees C. The resulting mixture was stirred for additional 5 h at 25 degrees C. LCMS indicated the reaction was completed. The reaction was quenched by the addition of water (20 mL) at room temperature. The resulting mixture was extracted with CH₂Cl₂ (3×100 mL). The residue was purified by silica gel column chromatography, eluted with PE/EtOAc (3:1) to afford chloromethyl 2-([[(tert-butoxy)carbonyl](methyl)amino]methyl)benzoate (Compound 8-4, 6.6 g, 65%) as a yellow oil. LCMS (ES, m/z): 314 [M+H]⁺, 214 [M+H−100]⁺. ¹H NMR (300 MHz, CDCl₃): 8.06 (t, J=3 Hz, 1H), 7.62-7.57 (m, 1H), 7.39-7.30 (m, 2H), 5.95 (s, 2H), 4.87 (s, 2H), 2.92 (d, J=3 Hz, 3H), 1.61-1.25 (m, 9H).

### Step 4. Synthesis of Compound 8-6

To a stirred mixture of 1-(3-chloro-4-methylphenyl)-3-[[2-(2,6-dioxopiperidin-3-yl)-1-oxo-2,3-dihydro-1H-isoindol-5-yl]methyl]urea (Compound 8-5, prepared according to the procedure described for Compound 1-5, 500 mg, 1.13 mmol, 1.00 equiv) and K₂CO₃ (470 mg, 3.40 mmol, 3.00 equiv) in DMF (10 mL) was added chloromethyl 2-([[(tert-butoxy)carbonyl](methyl)amino]methyl)benzoate (Compound 8-4, 712 mg, 2.29 mmol, 2.00 equiv) at room temperature. The resulting mixture was stirred for 16 h at room temperature. Desired product could be detected by LCMS. The residue was purified by reverse flash chromatography with the following conditions: column, C18 silica gel; mobile phase TFA, ACN in water, 10% to 80% gradient in 40 min; detector, UV 254 nm. The collected fraction was concentrated to afford [3-[5-([[(3-chloro-4-methylphenyl)carbamoyl]amino]methyl)-1-oxo-2,3-dihydro-1H-isoindol-2-yl]-2,6-dioxopiperidin-1-yl]methyl 2-([[(tert-butoxy)carbonyl](methyl)amino]-methyl)benzoate (Compound 8-6, 200 mg, 24%) as a semi-solid. LCMS (ES, m/z): 618,620 [M+H−100]⁺, 718, 720 [M+H]f.

### Step 5. Synthesis of Compound 8-7

To a stirred mixture of [3-[5-([[(3-chloro-4-methylphenyl)carbamoyl]amino]methyl)-1-oxo-3H-isoindol-2-yl]-2,6-dioxopiperidin-1-yl]methyl 2-[[(tert-butoxycarbonyl)(methyl)-amino]methyl]benzoate (Compound 8-6, 200 mg, 0.28 mmol, 1.00 equiv) in HCl (gas) in 1,4-dioxane (4 mL) in portions at room temperature under nitrogen atmosphere. The resulting mixture was stirred for 1 h at room temperature under nitrogen atmosphere. LCMS indicated the reaction was completed. The resulting mixture was concentrated under vacuum. The residue was purified by reverse flash chromatography with the following conditions: column, C18 silica gel; mobile phase TFA, ACN in water, 10% to 50% gradient in 30 min; detector, UV 254 nm. The mixture was lyophilized to afford [3-[5-([[(3-chloro-4-methylphenyl)carbamoyl]amino]methyl)-1-oxo-3H-isoindol-2-yl]-2,6-dioxopiperidin-1-yl]methyl 2-[(methylamino)methyl]benzoate (Compound 8-7, 80 mg, 41%) as a white solid. LCMS (ES, m/z): 618,620 [M+H]^{m}, 640, 642 [M+Na]f.

### Step 6. Synthesis of Compound 8-9

To a stirred mixture of (2S)-2-(2-[2-[(tert-butoxycarbonyl)amino]acetamido]-acetamido)-3-phenylpropanoic acid (Compound 8-8, 1.50 g, 3.95 mmol, 1.00 equiv) in DMF (15 mL) was added HATU (2.25 g, 5.92 mmol, 1.50 equiv), HOBT (0.53 g, 3.92 mmol, 0.99 equiv), glycine (0.36 g, 4.79 mmol, 1.21 equiv) and DIEA (1.53 g, 11.84 mmol, 2.99 equiv) at 0° C. The resulting mixture was stirred for overnight at 25° C. LCMS detected 12% desired product. The reaction mixture was purified by reverse flash chromatography with the following conditions: column, C18 silica gel; mobile phase FA (0.1%), ACN in water, 10% to 50% gradient in 40 min; detector, UV 254 nm. The resulting mixture was concentrated under vacuum to afford [(2S)-2-(2-[2-[(tert-butoxycarbonyl)amino]acetamido]acetamido)-3-phenylpropanamido]acetic acid (Compound 8-9, 300 mg, 15%) as a white solid. LCMS (ES, m/z): 437 [M+H]⁺, 337 [M+H−100]⁺.

### Step 7. Synthesis of Compound 8-10

To a stirred mixture of [(2S)-2-(2-[2-[(tert-butoxycarbonyl)amino]-acetamido]acetamido)-3-phenylpropanamido]acetic acid (Compound 8-9, 290 mg, 0.66 mmol, 1.00 equiv) in HCl (gas) in 1,4-dioxane (6.0 mL) at 0° C. The resulting mixture was stirred for additional 3 h at 25 degrees C. LCMS indicated the reaction was completed. The resulting mixture was concentrated under vacuum to afford [(2S)-2-[2-(2-aminoacetamido)acetamido]-3-phenylpropanamido]acetic acid hydrochloride (Compound 8-10, 330 mg, 93%) as a white solid. The crude product was used to next step without any purification. LCMS (ES, m/z): 337 [M+H]⁺.

### Step 8. Synthesis of Compound 8-12

To a stirred mixture of [(2S)-2-[2-(2-aminoacetamido)acetamido]-3-phenylpropanamido]acetic acid hydrochloride (Compound 8-10, 320 mg, 0.86 mmol, 1.00 equiv) in DMSO (6 mL) was added 2,5-dioxopyrrolidin-1-yl 6-(2,5-dioxopyrrol-1-yl)hexanoate (Compound 8-11, 318 mg, 1.03 mmol, 1.20 equiv) and DIEA (333 mg, 2.58 mmol, 3.0 equiv) in portions at room temperature under nitrogen atmosphere. The resulting mixture was stirred for additional 3 h at room temperature. LCMS indicated the reaction was completed. The residue was purified by reverse flash chromatography with the following conditions: column, C18 silica gel; mobile phase TFA (0.5%), ACN in water, 10% to 50% gradient in 30 min; detector, UV 254 nm. The collected fraction was lyophilized to afford [(2S)-2-(2-[2-[6-(2,5-dioxopyrrol-1-yl)hexanamido]acetamido]acetamido)-3-phenylpropanamido]acetic acid (Compound 8-12, 330 mg, 68%) as a white solid. LCMS (ES, m/z): 530 [M+H]⁺, 552 [M+Na]⁺. ¹H-NMR (300 MHz, DMSO-d₆) δ: 8.36-8.30 (m, 1H), 8.11-8.06 (m, 2H), 8.06-7.97 (m, 1H), 7.26-7.15 (m, 5H), 6.99 (s, 2H), 4.57-4.49 (m, 1H), 3.79-3.59 (m, 6H), 3.37 (t, J=6 Hz, 2H), 3.04 (t, J=9 Hz, 1H), 2.82-2.77 (m, 1H), 2.11 (t, J=9 Hz, 2H), 1.52-1.44 (m, 4H), 1.24-1.16 (m, 2H).

### Step 9. Synthesis of Compound (VIII)

To a stirred mixture of [(2S)-2-(2-[2-[6-(2,5-dioxopyrrol-1-yl)hexanamido]-acetamido]acetamido)-3-phenylpropanamido]acetic acid (Compound 8-7, 60 mg, 0.11 mmol, 1.00 equiv) and HATU (65 mg, 0.17 mmol, 1.50 equiv) in DMF (2 mL) were added HOBT (15 mg, 0.11 mmol, 1.0 equiv), [3-[5-([[(3-chloro-4-methylphenyl)carbamoyl]amino]methyl)-1-oxo-3H-isoindol-2-yl]-2,6-dioxopiperidin-1-yl]methyl 2-[(methylamino)methyl]benzoate (70 mg, 0.11 mmol, 1.00 equiv) and DIEA (44 mg, 0.34 mmol, 3.0 equiv) in portions at room temperature under nitrogen atmosphere. The resulting mixture was stirred for 2 h at room temperature under nitrogen atmosphere. LCMS indicated the reaction was completed. The crude product was purified by Prep-HPLC with the following conditions (Column: YMC-Actus Triart C18, 30 mm×150 mm, Sum; Mobile Phase A: Water (0.05% TFA), Mobile Phase B: ACN; Flow rate: 60 mL/min). The collected fraction was lyophilized to afford [3-[5-([[(3-chloro-4-methylphenyl)carbamoyl]amino]methyl)-1-oxo-3H-isoindol-2-yl]-2,6-dioxopiperidin-1-yl]methyl 2-([2-[(2S)-2-(2-[2-[6-(2,5-dioxopyrrol-1-yl)hexanamido]acetamido]acetamido)-3-phenylpropanamido]-N-methylacetamido]methyl)benzoate (Compound (VIII), 40 mg, 30%) as a white solid. LCMS (ES, m/z): 566 [M/2+1]⁺, 1129, 1131 [M+1]⁺, 1151, 1153 [M+Na]⁺. ¹H-NMR (300 MHz, DMSO-d₆) δ: 8.77 (s, 1H), 8.28-7.80 (m, 5H), 7.73-7.40 (m, 6H), 7.30-7.10 (m, 8H), 6.99 (s, 2H), 6.85-6.80 (m, 1H), 5.95-5.84 (m, 2H), 5.36-5.30 (m, 1H), 4.88-4.82 (m, 2H), 4.62-4.25 (m, 5H), 4.12 (d, J=3 Hz, 1H), 3.89 (d, J=3 Hz, 1H), 3.70-3.65 (m, 5H), 3.36 (t, J=6 Hz, 2H), 3.20-2.70 (m, 7H), 2.23 (s, 3H), 2.10 (t, J=9 Hz, 3H), 1.50-1.44 (m, 4H), 1.28-1.10 (m, 2H).

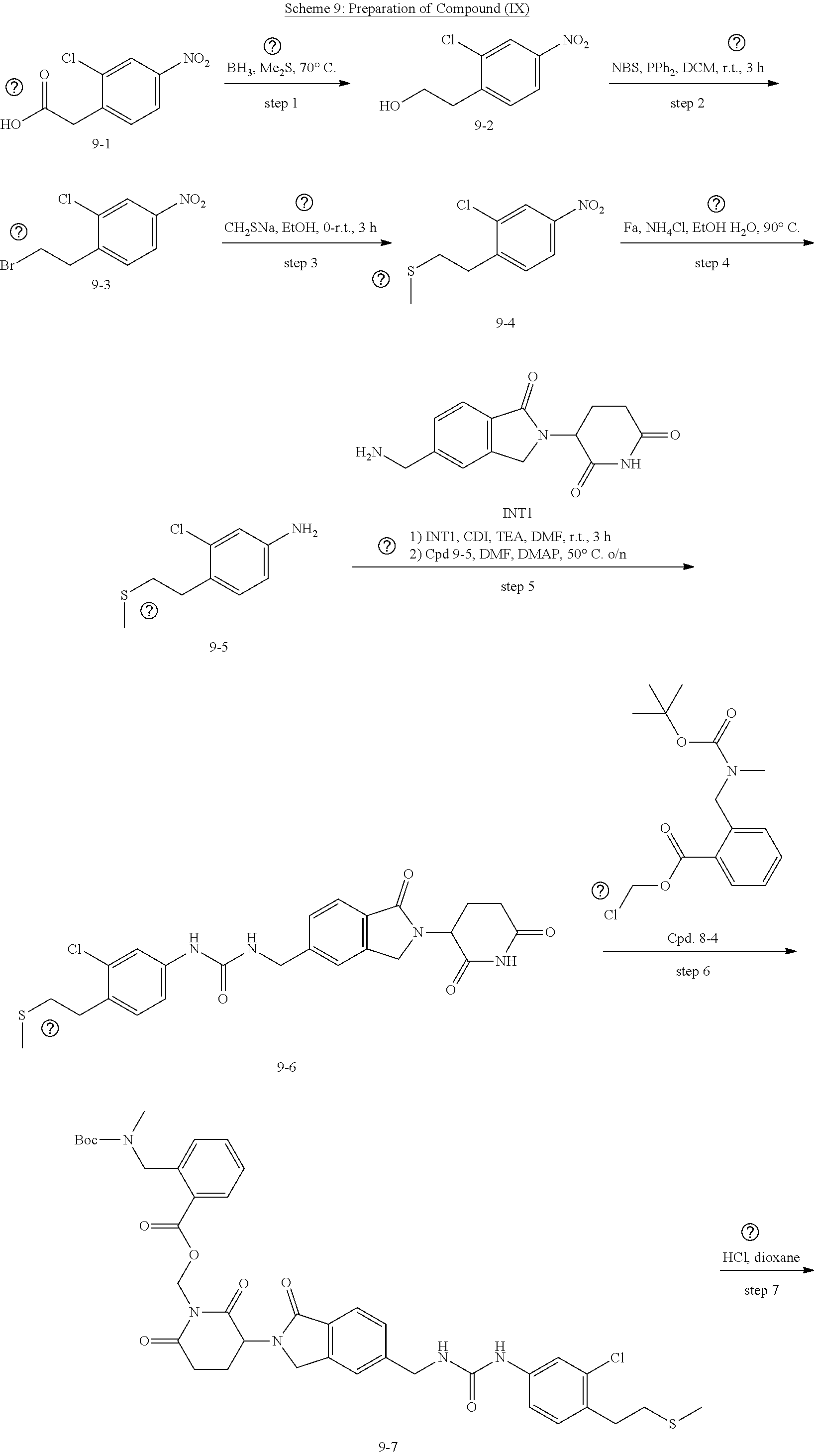

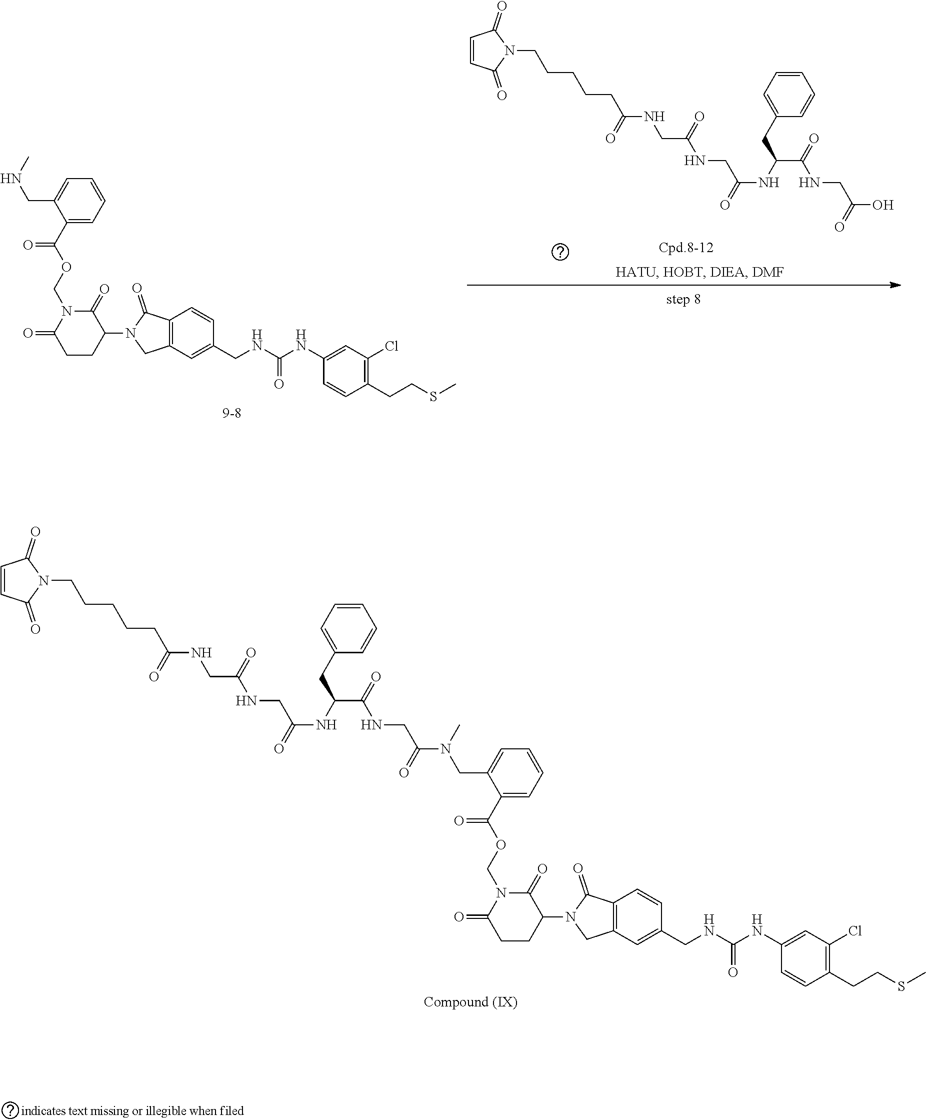 [SMILES: CNCc1ccccc1C(=O)OCN1C(=O)CCC(N2Cc3cc(CNC(=O)Nc4ccc(CCSC)c(Cl)c4)ccc3C2=O)C1=O ; O=C(O)CNC(=O)[C@H](Cc1ccccc1)NC(=O)CNC(=O)CNC(=O)CCCCCN1C(=O)C=CC1=O ; CSCCc1ccc(NC(=O)NCc2ccc3c(c2)CN(C2CCC(=O)N(COC(=O)c4ccccc4CN(C)C(=O)CNC(=O)[C@H](Cc4ccccc4)NC(=O)CNC(=O)CNC(=O)CCCCCN4C(=O)C=CC4=O)C2=O)C3=O)cc1Cl]

### Step 1. Synthesis of Compound 9-2

To a stirred solution of (2-chloro-4-nitrophenyl)acetic acid (Compound 9-1, 10 g, 46.38 mmol, 1.00 equiv) in THF (100 mL) were added BH₃-Me₂S (8.8 g, 115.97 mmol, 2.50 equiv) in portions at room temperature under nitrogen atmosphere. The resulting mixture was stirred for 2 h at 70° C. under nitrogen atmosphere. TLC indicated the reaction was completed. The reaction mixture was cooled down to room temperature and concentrated to dryness. The residue was purified by silica gel column chromatography, eluted with PE/EtOAc (2.1) to afford 2-(2-chloro-4-nitrophenyl)ethanol (Compound 9-2, 6.4 g, 68%) as a red oil. ¹H NMR (300 MHz, CDCl₃) δ 8.22 (s, 1H), 8.07-8.03 (m, 1H), 7.51 (d, J=3 Hz, 1H), 3.92 (t, J=6 Hz, 2H), 3.09 (t, J=6 Hz, 2H).

### Step 2. Synthesis of Compound 9-3

To a stirred solution of 2-(2-chloro-4-nitrophenyl)ethanol (Compound 9-2, 6.4 g, 31.74 mmol, 1.00 equiv) in DCM (120 mL) were added NBS (8.48 g, 47.64 mmol, 1.50 equiv) and PPh₃ (12.50 g, 47.62 mmol, 1.50 equiv) in portions at room temperature. The resulting mixture was stirred for overnight at room temperature. TLC traces indicated the reaction was completed. The reaction was concentrated to dryness under vaccum. The residue was purified by silica gel column chromatography, eluted with PE/EtOAc (4:1) to afford 1-(2-bromoethyl)-2-chloro-4-nitrobenzene (7.0 g, 66%) as a red oil. ¹H NMR (Compound 9-3, 300 MHz, CDCl₃) δ 8.28 (d, J=2.4 Hz, 1H), 8.13 (d, J=9.0 Hz, 1H), 7.51 (d, J=3 Hz, 1H), 3.66 (t, J=6.0 Hz, 2H), 3.42 (t, J=6.0 Hz, 2H).

### Step 3. Synthesis of Compound 9-4

To a stirred mixture of 1-(2-bromoethyl)-2-chloro-4-nitrobenzene (Compound 9-3, 6 g, 22.68 mmol, 1.00 equiv) in EtOH (60 mL) was added sodium methanethiolate (1.92 g, 27.45 mmol, 1.21 equiv) in portions at 0° C. under nitrogen atmosphere. The resulting mixture was stirred for 3 h at room temperature under nitrogen atmosphere. No desired product could be detected by LCMS, but TLC (PE:EA=10:1) showed a new point. The resulting mixture was concentrated under vacuum. The residue was purified by silica gel column chromatography, eluted with PE/EtOAc (9:1) to afford 2-chloro-1-[2-(methylsulfanyl)ethyl]-4-nitrobenzene (Compound 9-4, 1.7 g, 32%) as a yellow solid. ¹H NMR (300 MHz, CDCl₃): 8.28 (t, J=3 Hz, 1H), 8.10 (d, J=3 Hz, 1H), 7.45 (d, J=9 Hz, 1H), 3.14 (t, J=6 Hz, 2H), 2.80 (t, J=3 Hz, 2H), 2.18 (s, 3H).

### Step 4. Synthesis of Compound 9-5

To a stirred mixture of 2-chloro-1-[2-(methylsulfanyl)ethyl]-4-nitrobenzene (Compound 9-4, 2 g, 8.63 mmol, 1.00 equiv) and Fe (1.45 g, 25.96 mmol, 3.01 equiv) in EtOH (60 mL) was added NH₄Cl (4.6 g, 86.32 mmol, 10.00 equiv) in H₂O (20 mL). The resulting mixture was stirred for 3 h at 90° C. LCMS indicated the reaction was completed. The mixture was allowed to cool down to room temperature. The resulting mixture was filtered, the filter cake was washed with CH₂Cl₂. The resulting mixture was concentrated under vacuum. The residue was purified by reverse flash chromatography with the following conditions: column, C18 silica gel; mobile phase TFA (0.05%), ACN in water, 10% to 50% gradient in 40 min; detector, UV 254 nm. The collected fraction was concentrated to afford 3-chloro-4-[2-(methylsulfanyl)ethyl]aniline (Compound 9-5, 2.0 g, 69%) as a yellow oil. LCMS(ESI, ms): 202,204 [M+H]⁺, 243, 245 [M+H+ACN]⁺.

### Step 5. Synthesis of Compound 9-6

To a stirred mixture of 3-[5-(aminomethyl)-1-oxo-3H-isoindol-2-yl]piperidine-2,6-dione (INT1, prepared according to the procedure described for Compound 1-4, 1.4 g, 4.96 mmol, 1.00 equiv) in DMF (25 mL) were added CDI (0.80 g, 4.96 mmol, 1.00 equiv) and TEA (0.50 g, 4.94 mmol, 1.00 equiv) in portions at 0° C. under nitrogen atmosphere. The resulting mixture was stirred for 3 h at room temperature under nitrogen atmosphere. To the above mixture was added 3-chloro-4-[2-(methylsulfanyl)ethyl]aniline (compound 9-5, 1 g, 4.96 mmol, 1.00 equiv) and DMAP (1.82 g, 14.90 mmol, 3.00 equiv) in portions at room temperature. The resulting mixture was stirred for overnight at 60° C. under nitrogen atmosphere. 58% desired product could be detected by LCMS. The reaction mixture was purified by reverse flash chromatography with the following conditions: column, C18 silica gel; mobile phase TFA (0.05%), ACN in water, 10% to 70% gradient in 40 min; detector, UV 254 nm. The collected fraction was concentrated to afford 1-[3-chloro-4-[2-(methylsulfanyl)ethyl]phenyl]-3-[[2-(2,6-dioxopiperidin-3-yl)-1-oxo-3H-isoindol-5-yl]methyl]urea (Compound 9-6, 700 mg, 27%) as a solid. LCMS (ES, m/z): 501,503 [M+H]⁺. ¹H-NMR (300 MHz, DMSO-d₆) δ 10.98 (s, 1H), 8.79 (s, 1H), 7.71-7.67 (m, 2H), 7.52-7.25 (m, 2H), 7.25-7.15 (m, 2H), 6.82 (t, J=6 Hz, 1H), 5.14-5.08 (m, 1H), 4.49-4.29 (m, 4H), 2.98-2.84 (m, 3H), 2.84-2.63 (m, 3H), 2.42-2.34 (m, 1H), 2.09 (s, 3H), 2.03-1.90 (m, 1H).

### Step 6. Synthesis of Compound 9-7

To a stirred mixture of 1-[3-chloro-4-[2-(methylsulfanyl)ethyl]phenyl]-3-[[2-(2,6-dioxopiperidin-3-yl)-1-oxo-3H-isoindol-5-yl]methyl]urea (Compound 9-6, 700 mg, 1.40 mmol, 1.00 equiv) and K₂CO₃ (579 mg, 4.19 mmol, 3.00 equiv) in DMF (10 mL) were added chloromethyl 2-[[(tert-butoxycarbonyl)(methyl)amino]methyl]benzoate (Compound 8-4, 526 mg, 1.68 mmol, 1.20 equiv) in portions at room temperature under nitrogen atmosphere. The resulting mixture was stirred for 2d at room temperature under nitrogen atmosphere. LCMS indicated the reaction was completed. The residue was purified by reverse flash chromatography with the following conditions: column, C18 silica gel; mobile phase TFA (0.05%), ACN in water, 30% to 80% gradient in 40 min; detector, UV 254 nm. The collected fraction was lyophilized to afford [3-(5-[[([3-chloro-4-[2-(methylsulfanyl)ethyl]phenyl]carbamoyl)amino]methyl]-1-oxo-3H-isoindol-2-yl)-2,6-dioxopiperidin-1-yl]methyl 2-[[(tert-butoxycarbonyl)(methyl)amino]methyl]benzoate (Compound 9-7, 260 mg, 21%) as a white solid. LCMS (ES, m/z): 778,780 [M+H]⁺, 678, 780 [M+H−100]⁺.

### Step 7. Synthesis of Compound 9-8

To a stirred mixture of [3-(5-[[([3-chloro-4-[2-(methylsulfanyl)ethyl]phenyl]carbamoyl)amino]methyl]-1-oxo-3H-isoindol-2-yl)-2,6-dioxopiperidin-1-yl]methyl 2-[[(tert-butoxycarbonyl)(methyl)amino]methyl]benzoate (Compound 9-7, 250 mg, 0.32 mmol, 1.00 equiv) in HCl (gas) in 1,4-dioxane (5 mL) in portions at room temperature under nitrogen atmosphere. The resulting mixture was stirred for 1 h at room temperature under nitrogen atmosphere. LCMS indicated the reaction was completed. The resulting mixture was concentrated under vacuum. The crude product was purified by reverse flash chromatography with the following conditions: column, C18 silica gel; mobile phase TFA (0.05%), ACN in water, 10% to 50% gradient in 40 min; detector, UV 254 nm. The mixture was lyophilized to afford [3-(5-[[([3-chloro-4-[2-(methylsulfanyl)ethyl]phenyl]carbamoyl)amino]methyl]-1-oxo-3H-isoindol-2-yl)-2,6-dioxopiperidin-1-yl]methyl 2-[(methylamino)methyl]benzoate (Compound 9-8, 132 mg, 56%) as a yellow solid. LCMS (ES, m/z): 678,680 [M+H]⁺, 700, 702 [M+Na]⁺.

### Step 8. Synthesis of Compound (IX)

To a stirred mixture of [(2S)-2-(2-[2-[6-(2,5-dioxopyrrol-1-yl)hexanamido]acetamido]acetamido)-3-phenylpropanamido]acetic acid (Compound 8-12, 100 mg, 0.19 mmol, 1.00 equiv) and HATU (108 mg, 0.28 mmol, 1.5 equiv) in DMF (2.00 mL) were added HOBT (26 mg, 0.19 mmol, 1.0 equiv), [3-(5-[[([3-chloro-4-[2-(methylsulfanyl)ethyl]phenyl]carbamoyl)amino]methyl]-1-oxo-3H-isoindol-2-yl)-2,6-dioxopiperidin-1-yl]methyl 2-[(methylamino)methyl]benzoate (Compound 9-8, 115 mg, 0.17 mmol, 0.90 equiv) and DIEA (73 mg, 0.57 mmol, 3.0 equiv) in portions at room temperature under nitrogen atmosphere. The resulting mixture was stirred for 2 h at room temperature under nitrogen atmosphere. LCMS indicated the reaction was completed. The crude product was purified by Prep-HPLC with the following conditions (Column: XSelect CSH Prep C18 OBD Column, 19×250 mm, 5 um; Mobile Phase A: water (0.05% TFA), Mobile Phase B: ACN; Flow rate: 25 mL/min). The collected fraction was lyophilized to afford [3-(5-[[([3-chloro-4-[2-(methylsulfanyl)ethyl]phenyl]carbamoyl)amino]methyl]-1-oxo-3H-isoindol-2-yl)-2,6-dioxopiperidin-1-yl]methyl 2-([2-[(2S)-2-(2-[2-[6-(2,5-dioxopyrrol-1-yl)hexanamido]acetamido]acetamido)-3-phenylpropanamido]-N-methylacetamido]methyl)benzoate (Compound (IX), 52.1 mg, 23%) as a white solid. LCMS (ES, m/z): 596 [M/2+1]⁺, 1189, 1191 [M+1]⁺. ¹H-NMR (300 MHz, DMSO-d₆) δ 8.82 (s, 1H), 8.12-7.83 (m, 5H), 7.73-7.38 (m, 6H), 7.26-7.15 (m, 8H), 6.99 (s, 2H), 6.83 (t, J=6 Hz, 1H), 5.98-5.84 (m, 2H), 5.32 (t, J=6 Hz, 1H), 4.884.81 (m, 2H), 4.65-4.30 (m, 5H), 4.12 (d, J=3 Hz, 1H), 3.94-3.85 (m, 4H), 3.72-3.59 (m, 3H), 3.36 (t, J=6 Hz, 2H), 3.20-2.95 (m, 4H), 2.89-2.74 (m, 4H), 2.62-2.50 (m, 2H), 2.12-2.05 (m, 6H), 1.58-1.40 (m, 4H), 1.28-1.10 (m, 2H).

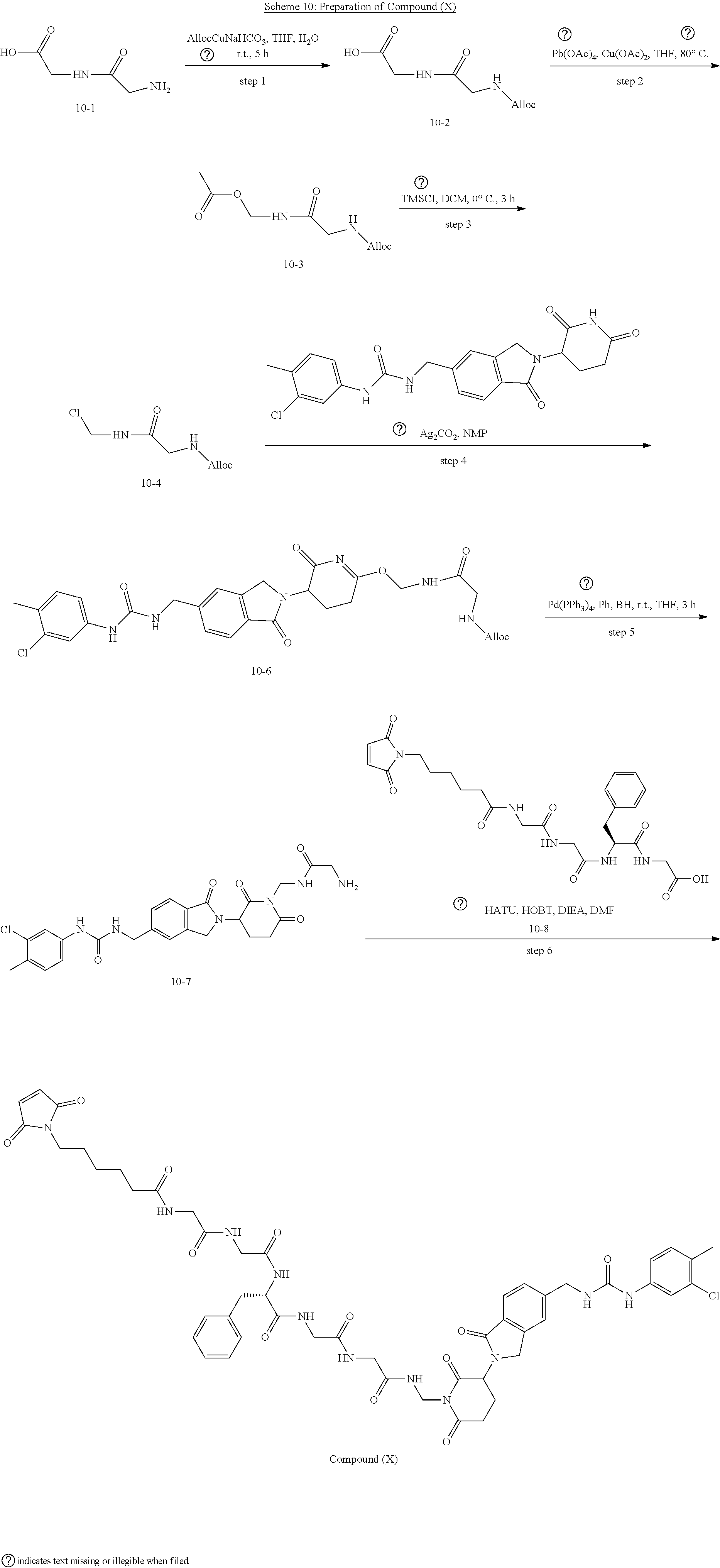

### Step 1. Synthesis of Compound 10-2

To a stirred mixture of Gly-Gly (10 g, 75.69 mmol, 1.00 equiv) and NaHCO₃ (12.72 g, 151.3 mmol, 2 equiv) in H₂O (70 mL) was added chloro(prop-2-en-1-yloxy)methanone (10.95 g, 90.82 mmol, 1.2 equiv) in THE (35 mL) dropwise at 0° C. The resulting mixture was stirred for 5 h at 25° C. LCMS indicated the reaction was completed. The resulting mixture was concentrated under reduced pressure. The mixture was acidified to pH 5 with HCl (aq. 1 N). The precipitated solids were collected by filtration and washed with HCl (aq. 1 N) (2×5 mL). This resulted in (2-{[(prop-2-en-1-yloxy)carbonyl]amino}acetamido)acetic acid (Compound 10-2, 9 g, 55%) as a white solid. LCMS (ES, m/z): 217 [M+H]⁺

### Step 2. Synthesis of Compound 10-3

A mixture of (2-{[(prop-2-en-1-yloxy)carbonyl]amino}acetamido)acetic acid (Compound 10-2, 9 g, 41.62 mmol, 1.00 equiv) and Cu(OAc)₂ (0.76 g, 4.16 mmol, 0.1 equiv) in THE (220 mL) was stirred for 1 h at 60° C. under nitrogen atmosphere. The mixture was allowed to cool down to room temperature. To the above mixture was added Pb(OAc)₄ (22.15 g, 49.9 mmol, 1.2 equiv) at room temperature. The resulting mixture was stirred for additional 1 h at 25° C. LCMS indicated the reaction was completed. The resulting mixture was filtered, the filter cake was washed with MeOH (3×50 mL). The filtrate was concentrated under reduced pressure. The residue was purified by silica gel column chromatography, eluted with PE/EA (1:10) to afford (2-{[(prop-2-en-1-yloxy)carbonyl]amino}acetamido)methyl acetate (Compound 10-3, 5 g, 52%) as a white solid. LCMS (ES, m/z): 231 [M+H]⁺

### Step 3. Synthesis of Compound 10-4

To a stirred mixture of (2-{[(prop-2-en-1-yloxy)carbonyl]amino}acetamido)methyl acetate (Compound 10-3, 1 g, 4.34 mmol, 1.00 equiv) in DCM (40 mL) was added TMSCl (1.89 g, 17.37 mmol, 4 equiv) dropwise at 0° C. The resulting mixture was stirred for 1 h at 0° C. LCMS (quenched with MeOH for LCMS) indicated the reaction was completed. The resulting mixture was concentrated under reduced pressure. The crude product was used in the next step directly without further purification. This resulted in prop-2-en-1-yl N-[(chloromethylcarbamoyl)methyl]carbamate (Compound 10-3, 1 g, 77%) as a yellow solid. LCMS (ES, m/z): 203 [M+H]⁺ (quenched with MeOH)

### Step 4. Synthesis of Compound 10-6

A mixture of 1-(3-chloro-4-methylphenyl)-3-{[2-(2,6-dioxopiperidin-3-yl)-1-oxo-3H-isoindol-5-yl]methyl}urea (Compound 10-5, prepared according to the procedure described for Compound 1-5, 750 mg, 1.70 mmol, 1.00 equiv) and Ag₂CO₃ (938 mg, 3.40 mmol, 2 equiv) in NMP (15.00 mL) was stirred for 1 h at 50° C. The mixture was allowed to cool down to room temperature. To the above mixture was added prop-2-en-1-yl N-[(chloromethylcarbamoyl)methyl]carbamate (Compound 10-4, 703 mg, 3.40 mmol, 2.00 equiv) at room temperature. The resulting mixture was stirred for additional 36 h at 60° C. LCMS indicated the reaction was completed. The residue was purified by YMC-Actus Triart C18 ExRS, 30×150 mm; Mobile Phase A: water (0.05% TFA), Mobile Phase B: ACN; Flow rate: 60 mL/min; Gradient: 27% B to 53% B in 10 min, 53% B; Wave Length: 254 nm; RT1 (min): 9.22 min. This resulted in prop-2-en-1-yl N-{[({3-[5-({[(3-chloro-4-methylphenyl)carbamoyl]amino}methyl)-1-oxo-3H-isoindol-2-yl]-2,6-dioxopiperidin-1-yl}methyl)carbamoyl]methyl}carbamate (Compound 10-6, 100 mg, 8%) as a yellow solid. LCMS (ES, m/z): 611,613 [M+H]⁺

### Step 5. Synthesis of Compound 10-7

To a stirred mixture of prop-2-en-1-yl N-{[({3-[5-({[(3-chloro-4-methylphenyl)carbamoyl]amino}methyl)-1-oxo-3H-isoindol-2-yl]-2,6-dioxopiperidin-1-yl}methyl)carbamoyl]methyl}carbamate (Compound 10-6, 100 mg, 0.17 mmol, 1.00 equiv) and Pd(PPh₃)₄ (19 mg, 0.017 mmol, 0.10 equiv) in THE (1.50 mL) was added phenylsilane (36 mg, 0.34 mmol, 2.00 equiv) dropwise at 25° C. under nitrogen atmosphere. The resulting mixture was stirred for 2 h at 25° C. under nitrogen atmosphere. LCMS indicated the reaction was completed. The reaction mixture was purified by reverse flash chromatography with the following conditions: column, C18 silica gel; mobile phase, water (0.05% TFA), ACN (5% to 50% gradient in 30 min); detector, UV 254 nm. This resulted in 2-amino-N-({3-[5-({[(3-chloro-4-methylphenyl)carbamoyl]amino}methyl)-1-oxo-3H-isoindol-2-yl]-2,6-dioxopiperidin-1-yl}methyl)acetamide (Compound 10-7, 70 mg, 79%) as a yellow solid. LCMS (ES, m/z): 527,529 [M+H]⁺

### Step 6. Synthesis of Compound (X)

To a stirred mixture of [(2S)-2-(2-{2-[6-(2,5-dioxopyrrol-1-yl)hexanamido]acetamido}acetamido)-3-phenylpropanamido]acetic acid (Compound 10-8, prepared according to the procedure described for Compound 8-12, 65 mg, 0.12 mmol, 1 equiv) and HATU (56 mg, 0.14 mmol, 1.2 equiv), HOBT (20 mg, 0.14 mmol, 1.2 equiv) in DMF (650 uL) was added 2-amino-N-({3-[5-({[(3-chloro-4-methylphenyl)carbamoyl]amino}methyl)-1-oxo-3H-isoindol-2-yl]-2,6-dioxopiperidin-1-yl}methyl)acetamide (Compound 10-7, 65 mg, 0.12 mmol, 1.00 equiv) and DIEA (47 mg, 0.36 mmol, 3 equiv) at 0° C. The resulting mixture was stirred for 16 h at 25° C. LCMS indicated the reaction was completed. The reaction mixture was purified by reverse flash chromatography with the following conditions: column, C18 silica gel; mobile phase, water (containing 0.5% TFA), ACN 10% to 50% gradient in 30 min; detector, UV 254 nm. The crude product was re-purified by Prep-HPLC with the following conditions Column: Kinetex EVO prep C18, 30×150, 5 um; Mobile Phase A: water (0.05% TFA), Mobile Phase B: ACN; Flow rate: 60 mL/min; Gradient: 25% B to 35% B in 17 min, 35% B; Wave Length: 254 nm; RT1 (min): 16. The collected fraction was lyophilized to afford N-{[({[(1S)-1-{[({[({3-[5-({[(3-chloro-4-methylphenyl)carbamoyl]amino}methyl)-1-oxo-3H-isoindol-2-yl]-2,6-dioxopiperidin-1-yl}methyl)carbamoyl]methyl}carbamoyl)methyl]carbamoyl}-2-phenylethyl]carbamoyl}methyl)carbamoyl]methyl}-6-(2,5-dioxopyrrol-1-yl)hexanamide (Compound (X), 11.4 mg, 8.84%) as a white solid. LCMS (ES, m/z): 1038, 1040 [M+H]⁺. ¹H-NMR (DMSO, 400 MHz) δ (ppm): 8.75 (s, 1H), 8.31-8.29 (m, 1H), 8.19-8.16 (m, 1H), 8.12-8.05 (m, 2H), 7.99-7.94 (m, 2H), 7.71-7.66 (m, 2H), 7.52 (s, 1H), 7.44 (d, J=8.0 Hz, 1H), 7.24-7.08 (m, 7H), 6.99-6.86 (m, 2H), 6.82-6.79 (m, 1H), 5.20-5.13 (m, 2H), 4.99-4.95 (m, 1H), 4.49-4.40 (m, 4H), 4.31-4.27 (m, 1H), 3.76-3.56 (m, 8H), 3.37-3.30 (m, 2H), 3.07-2.97 (m, 2H), 2.79-2.67 (m, 2H), 2.40-2.30 (m, 1H), 2.22 (s, 3H), 2.11-2.02 (m, 3H), 1.49-1.42 (m, 4H), 1.23-1.14 (m, 2H).

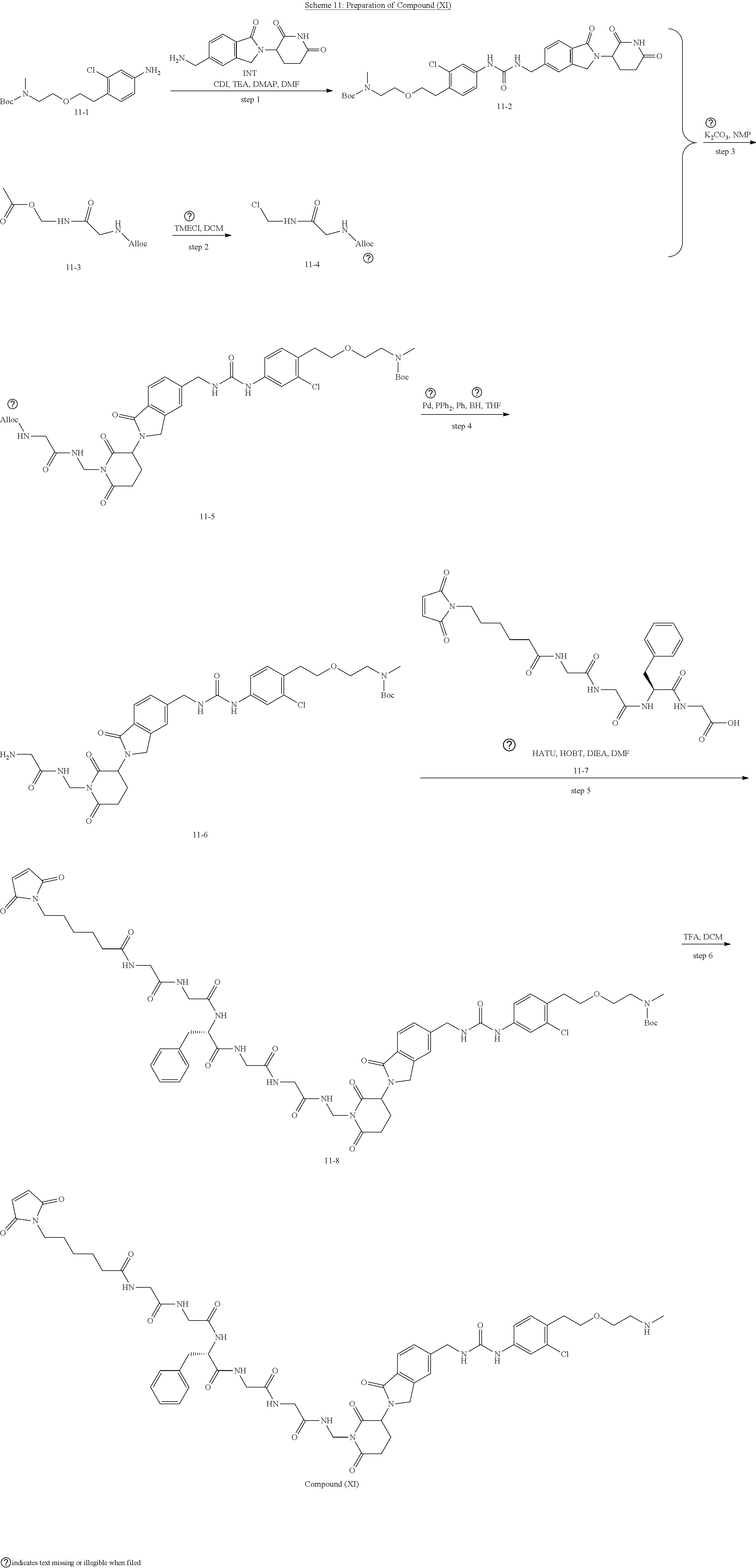

### Step 1. Synthesis of Compound 11-2

To a stirred mixture of 3-[5-(aminomethyl)-1-oxo-3H-isoindol-2-yl]piperidine-2,6-dione (INT, prepared according to the procedure described for Compound 1-4, 1.99 g, 7.29 mmol, 1.2 equiv) in DMF (20 mL) was added CDI (0.99 g, 6.08 mmol, 1 equiv), TEA (0.62 g, 6.08 mmol, 1 equiv) at 0° C. under nitrogen atmosphere. The resulting mixture was stirred for 1 h at 0° C. under nitrogen atmosphere. To the above mixture was added tert-butyl N-{2-[2-(4-amino-2-chlorophenyl)ethoxy]ethyl}-N-methylcarbamate (Compound 11-1, 2 g, 6.08 mmol, 1.00 equiv) at 0° C. The resulting mixture was stirred for additional 16 h at 60° C. LCMS indicated 60% of desired product. The mixture was allowed to cool down to room temperature. The reaction was quenched by the addition of water (20 mL) at room temperature. The resulting mixture was extracted with EtOAc (3×30 mL). The combined organic layers were washed with brine (30 mL), dried over anhydrous Na₂SO₄. After filtration, the filtrate was concentrated under reduced pressure. The residue was purified by reverse flash chromatography with the following conditions: column, C18 silica gel; mobile phase, water (0.05% TFA), ACN (5% to 50% gradient in 30 min); detector, UV 254 nm. This resulted in tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 11-2, 2 g, 52%) as a yellow solid. LCMS (ES, m/z): 628,630 [M+H]⁺

### Step 2. Synthesis of Compound 11-4

To a stirred mixture of (2-{[(prop-2-en-1-yloxy)carbonyl]amino}acetamido)methyl acetate (Compound 11-3, 1 g, 4.34 mmol, 1.00 equiv) in DCM (40 mL) was added TMSCl (1.89 g, 17.37 mmol, 4 equiv) dropwise at 0° C. The resulting mixture was stirred for 2 h at 0° C. LCMS indicated the reaction was completed. The resulting mixture was concentrated under reduced pressure. This resulted in prop-2-en-1-yl N-[(chloromethylcarbamoyl)methyl]carbamate (Compound 11-4, 0.8 g, 89%) as a white solid. LCMS (ES, m/z): 203 [M+H]⁺ (quenched with MeOH for LCMS)

### Step 3. Synthesis of Compound 11-5

To a stirred mixture of tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 11-2, 1 g, 1.59 mmol, 1.00 equiv) and K₂CO₃ (0.44 g, 3.18 mmol, 2 equiv) in NMP (16 mL) was added prop-2-en-1-yl N-[(chloromethylcarbamoyl)methyl]carbamate (Compound 11-4, 0.82 g, 3.98 mmol, 2.5 equiv) in portions at 25° C. The resulting mixture was stirred for 16 h at 60° C. LCMS indicated the reaction was completed. The mixture was allowed to cool down to room temperature. The reaction was quenched by the addition of water (20 mL) at room temperature. The resulting mixture was extracted with EtOAc (3×30 mL). The combined organic layers were washed with brine (30 mL), dried over anhydrous Na₂SO₄. After filtration, the filtrate was concentrated under reduced pressure. The residue was purified by reverse flash chromatography with the following conditions: column, C18 silica gel; mobile phase, water (0.05% TFA), ACN (5% to 50% gradient in 30 min; detector, UV 254 nm. This resulted in tert-butyl N-(2-{2-[2-chloro-4-({[(2-{2,6-dioxo-1-[(2-{[(prop-2-en-1-yloxy)carbonyl]amino}acetamido)methyl]piperidin-3-yl}-1-oxo-3H-isoindol-5-yl)methyl]carbamoyl}amino)phenyl]ethoxy}ethyl)-N-methylcarbamate Compound 11-5, (0.5 g, 39%) as a yellow solid. LCMS (ES, m/z): 798,800 [M+H]⁺

### Step 4. Synthesis of Compound 11-6

To a stirred mixture of tert-butyl N-(2-{2-[2-chloro-4-({[(2-{2,6-dioxo-1-[(2-{[(prop-2-en-1-yloxy)carbonyl]amino}acetamido)methyl]piperidin-3-yl}-1-oxo-3H-isoindol-5-yl)methyl]carbamoyl}amino)phenyl]ethoxy}ethyl)-N-methylcarbamate (Compound 11-5, 500 mg, 0.62 mmol, 1.00 equiv) in THE (6 mL) was added Pd(PPh₃)₄ (72 mg, 0.063 mmol, 0.1 equiv), phenylsilane (136 mg, 1.25 mmol, 2 equiv) at 0° C. under nitrogen atmosphere. The resulting mixture was stirred for 3 h at 25° C. under nitrogen atmosphere. LCMS indicated the reaction was completed. The mixture was allowed to cool down to room temperature. The reaction was quenched by the addition of water (20 mL) at room temperature. The resulting mixture was extracted with EtOAc (3×30 mL). The combined organic layers were washed with brine (30 mL), dried over anhydrous Na₂SO₄. After filtration, the filtrate was concentrated under reduced pressure. The residue was purified by reverse flash chromatography with the following conditions: column, C18 silica gel; mobile phase, water (0.05% TFA), ACN (5% to 50% gradient in 30 min); detector, UV 254 nm. This resulted in tert-butyl N-(2-{2-[4-({[(2-{1-[(2-aminoacetamido)methyl]-2,6-dioxopiperidin-3-yl}-1-oxo-3H-isoindol-5-yl)methyl]carbamoyl}amino)-2-chlorophenyl]ethoxy}ethyl)-N-methylcarbamate (Compound 11-6, 400 mg, 89%) as a yellow solid. LCMS (ES, m/z): 714,716 [M+H]+

### Step 5. Synthesis of Compound 11-8

To a stirred mixture of [(2S)-2-(2-{2-[6-(2,5-dioxopyrrol-1-yl)hexanamido]acetamido}acetamido)-3-phenylpropanamido]acetic acid (Compound 11-7, prepared according to the procedure described for Compound 8-12, 163 mg, 0.30 mmol, 1.1 equiv), HATU (159 mg, 0.42 mmol, 1.5 equiv) and HOBT (57 mg, 0.42 mmol, 1.5 equiv) in DMF (3 mL) was added tert-butyl N-(2-{2-[4-({[(2-{1-[(2-aminoacetamido)methyl]-2,6-dioxopiperidin-3-yl}-1-oxo-3H-isoindol-5-yl)methyl]carbamoyl}amino)-2-chlorophenyl]ethoxy}ethyl)-N-methylcarbamate (Compound 11-6, 200 mg, 0.28 mmol, 1.00 equiv), DIEA (108 mg, 0.84 mmol, 3 equiv) at 0° C. The resulting mixture was stirred for 5 h at 25° C. LCMS indicated the reaction was completed. The reaction was quenched by the addition of water (10 mL). The precipitated solids were collected by filtration and washed with water (11 mL). This resulted in tert-butyl N-(2-{2-[2-chloro-4-({[(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-1-oxo-3H-isoindol-5-yl)methyl]carbamoyl}amino)phenyl]ethoxy}ethyl)-N-methylcarbamate (Compound 11-8, 100 mg, 29%) as a yellow solid. LCMS (ES, m/z): 1225,1227 [M+H]⁺

### Step 6. Synthesis of Compound (XI)

To a stirred mixture of tert-butyl N-(2-{2-[2-chloro-4-({[(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-1-oxo-3H-isoindol-5-yl)methyl]carbamoyl}amino)phenyl]ethoxy}ethyl)-N-methylcarbamate (Compound 11-8, 100 mg, 0.08 mmol, 1.00 equiv) in DCM (0.8 mL) was added TFA (0.2 mL) at 0° C. The resulting mixture was stirred for 4 h at 25° C. LCMS indicated the reaction was completed. The resulting mixture was concentrated under reduced pressure. The crude product was purified by Prep-HPLC with the following conditions Column: XBridge Shield RP18 OBD Column, 30×150 mm, 5 m; Mobile Phase A: water (0.05% TFA), Mobile Phase B: ACN; Flow rate: 60 mL/min; Gradient: 20% B to 40% B in 7 min, 40% B; Wave Length: 254 nm; RT1 (min): 5.7 min. The collected fraction was lyophilized to afford N-{[({[(1S)-1-{[({[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)carbamoyl]methyl}carbamoyl)methyl]carbamoyl}-2-phenylethyl]carbamoyl}methyl)carbamoyl]methyl}-6-(2,5-dioxopyrrol-1-yl)hexanamide; trifluoroacetic acid (Compound (XI), 30.5 mg, 29%) as a white solid. LCMS (ES, m/z): 1125, 1127 [M+H]⁺. ¹H-NMR (DMSO, 400 MHz) δ (ppm): 8.88 (s, 1H), 8.39 (br s, 2H), 8.33-8.29 (m, 1H), 8.21-8.17 (m, 1H), 8.13-8.06 (m, 2H), 8.01-7.94 (m, 2H), 7.71 (d, J=7.6 Hz, 1H), 7.72 (d, J=2.0 Hz, 1H), 7.51 (s, 1H), 7.44 (d, J=8.0 Hz, 1H), 7.26-7.14 (m, 7H), 6.99 (s, 2H), 6.93-6.90 (m, 1H), 5.26-5.08 (m, 2H), 5.02-4.88 (m, 1H), 4.58-4.39 (m, 4H), 4.35-4.18 (m, 1H), 3.76-3.56 (m, 12H), 3.37-3.34 (m 2H), 3.10-3.00 (m 4H), 2.90-2.87 (m 2H), 2.81-2.75 (m, 2H), 2.57-2.54 (m, 3H), 2.42-2.32 (m 1H), 2.11-2.08 (m 2H), 2.06-1.99 (m 1H), 1.49-1.43 (m 4H), 1.19-1.16 (m 2H).

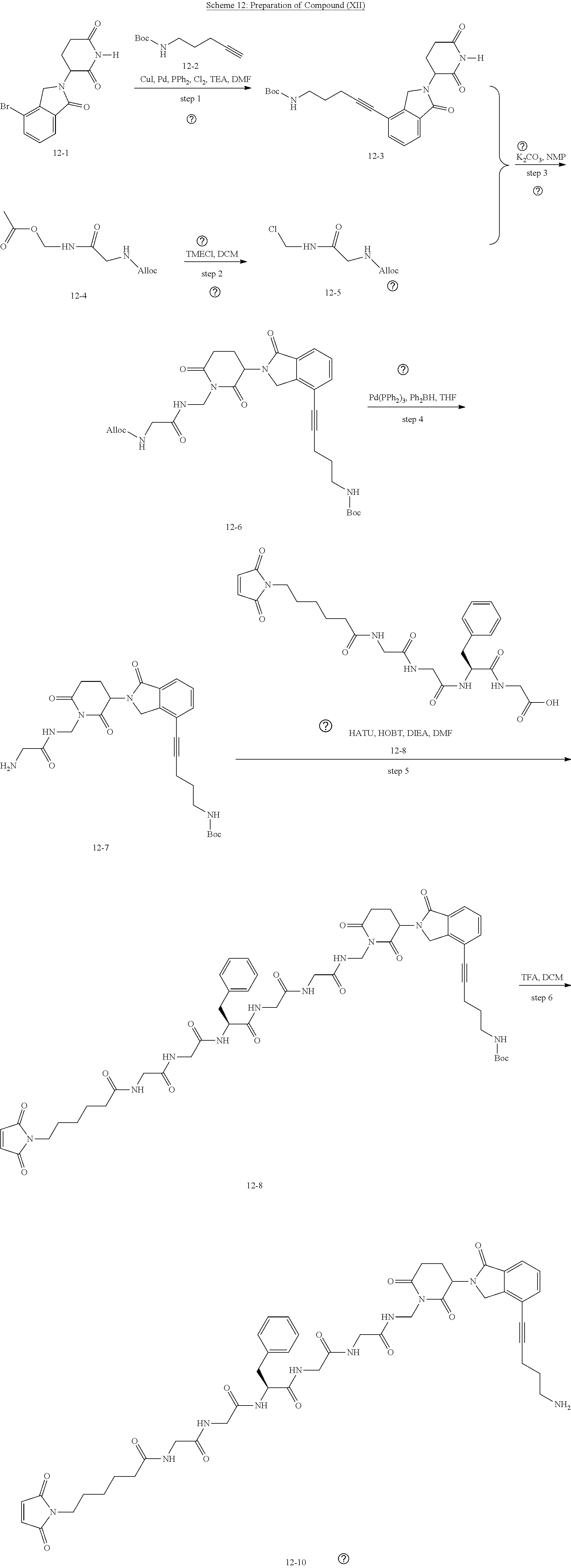

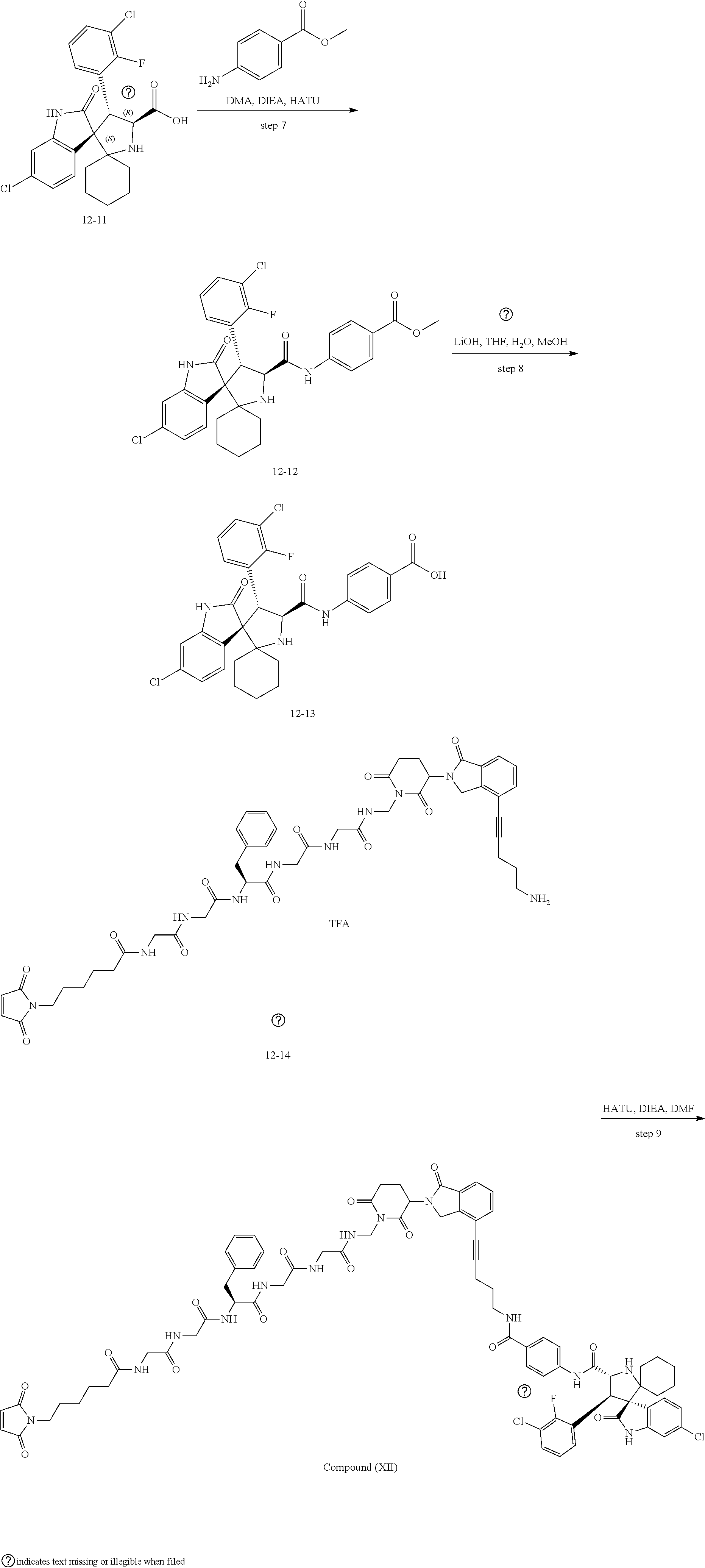

### Step 1. Synthesis of Compound 12-3

To a stirred mixture of 3-(4-bromo-1-oxo-3H-isoindol-2-yl)piperidine-2,6-dione (Compound 12-1, 3 g, 9.28 mmol, 1.00 equiv) and tert-butyl N-(pent-4-yn-1-yl)carbamate (Compound 12-2, 3.06 g, 16.71 mmol, 1.8 equiv) in DMF (31 mL) and TEA (31 mL) was added CuI (0.35 g, 1.85 mmol, 0.2 equiv) and Pd(PPh₃)₂Cl₂ (0.65 g, 0.92 mmol, 0.1 equiv) at room temperature under nitrogen atmosphere. The resulting mixture was stirred for 16 h at 80° C. under nitrogen atmosphere. LCMS indicated the reaction was completed. The mixture was allowed to cool down to room temperature. The reaction was quenched by the addition of water (100 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-{5-[2-(2,6-dioxopiperidin-3-yl)-1-oxo-3H-isoindol-4-yl]pent-4-yn-1-yl}carbamate (Compound 12-32 g, 50%) as a brown solid. LCMS (ES, m/z): 426 [M+H]⁺

### Step 2. Synthesis of Compound 12-5

To a stirred mixture of (2-{[(prop-2-en-1-yloxy)carbonyl]amino}acetamido)methyl acetate (Compound 12-4, prepared according to the procedure described for Compound 10-3, 0.9 g, 3.90 mmol, 1.00 equiv) in DCM (38 mL) was added TMSCl (1.9 mL, 14.86 mmol, 3.80 equiv) dropwise at 0° C. The resulting mixture was stirred for 2 h at 0° C. LCMS indicated the reaction was completed. The resulting mixture was concentrated under reduced pressure. The crude product was used in the next step directly without further purification. This resulted in prop-2-en-1-yl N-[(chloromethylcarbamoyl)methyl]carbamate (Compound 12-5, 0.8 g, 99%) as a white solid. LCMS (ES, m/z): 203 [M+H]⁺ (derivated with methanol)

### Step 3. Synthesis of Compound 12-6

To a stirred mixture of tert-butyl N-{5-[2-(2,6-dioxopiperidin-3-yl)-1-oxo-3H-isoindol-4-yl]pent-4-yn-1-yl}carbamate (Compound 12-3, 1 g, 2.35 mmol, 1.00 equiv) and prop-2-en-1-yl N-[(chloromethylcarbamoyl)methyl]carbamate (Compound 12-5, 0.73 g, 3.52 mmol, 1.5 equiv) in NMP (24 mL) was added K₂CO₃ (0.65 g, 4.70 mmol, 2 equiv) at room temperature. The resulting mixture was stirred for 16 h at 60° C. LCMS indicated the reaction was completed. The reaction was quenched by the addition of water (30 mL) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by reverse flash chromatography with the following conditions: column, C18 silica gel; mobile phase, water (0.05% TFA), ACN (5% to 50% gradient in 30 min; detector, UV 254 nm. This resulted in tert-butyl N-[5-(2-{2,6-dioxo-1-[(2-{[(prop-2-en-1-yloxy)carbonyl]amino}acetamido)methyl]piperidin-3-yl}-1-oxo-3H-isoindol-4-yl)pent-4-yn-1-yl]carbamate (Compound 12-6, 1.2 g, 85%) as a yellow oil. LCMS (ES, m/z): 596 [M+H]⁺

### Step 4. Synthesis of Compound 12-7

To a stirred mixture of tert-butyl N-[5-(2-{2,6-dioxo-1-[(2-{[(prop-2-en-1-yloxy)carbonyl]amino}acetamido)methyl]piperidin-3-yl}-1-oxo-3H-isoindol-4-yl)pent-4-yn-1-yl]carbamate (Compound 12-6, 1.4 g, 2.35 mmol, 1.00 equiv) and Pd(PPh₃)₄ (0.27 g, 0.23 mmol, 0.1 equiv) in THE (30 mL) was added phenylsilane (0.51 g, 4.70 mmol, 2 equiv) under nitrogen atmosphere. The resulting mixture was stirred for 3 h at 25° C. under nitrogen atmosphere. LCMS indicated the reaction was completed. The reaction was quenched by the addition of water (1 mL) at room temperature. The resulting mixture was concentrated under reduced pressure. The residue was purified by reverse flash chromatography with the following conditions: column, C18 silica gel; mobile phase, water (0.05% TFA), ACN 5% to 50% gradient in 30 min; detector, UV 254 nm. This resulted in tert-butyl N-[5-(2-{1-[(2-aminoacetamido)methyl]-2,6-dioxopiperidin-3-yl}-1-oxo-3H-isoindol-4-yl)pent-4-yn-1-yl]carbamate; trifluoroacetic acid (Compound 12-7, 500 mg, 34%) as a yellow solid. LCMS (ES, m/z): 512 [M+H]⁺

### Step 5. Synthesis of Compound 12-9

To a stirred mixture of [(2S)-2-(2-{2-[6-(2,5-dioxopyrrol-1-yl)hexanamido]acetamido}acetamido)-3-phenylpropanamido]acetic acid (Compound 12-8, prepared according to the procedure described for Compound 8-12, 279.33 mg, 0.52 mmol, 1.1 equiv) in DMF (6.00 mL) was added HATU (218.80 mg, 0.57 mmol, 1.2 equiv) and HOBT (77.76 mg, 0.57 mmol, 1.2 equiv) at 0° C. The resulting mixture was stirred for 30 min at 25° C. To the above mixture was added tert-butyl N-[5-(2-{1-[(2-aminoacetamido)methyl]-2,6-dioxopiperidin-3-yl}-1-oxo-3H-isoindol-4-yl)pent-4-yn-1-yl]carbamate; trifluoroacetic acid (Compound 12-7, 300 mg, 0.48 mmol, 1.00 equiv) and DIEA (185.93 mg, 1.44 mmol, 3 equiv) at 0° C. The resulting mixture was stirred for additional 16 h at 25° C. LCMS indicated the reaction was completed. The reaction was quenched by the addition of water (0.5 mL) at room temperature. The residue was purified by reverse flash chromatography with the following conditions: column, C18 silica gel; mobile phase, water (0.05% TFA), ACN (5% to 50% gradient in 30 min); detector, UV 254 nm. This resulted in tert-butyl N-[5-(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-1-oxo-3H-isoindol-4-yl)pent-4-yn-1-yl]carbamate (Compound 12-9, 300 mg, 61%) as a yellow solid. LCMS (ES, m/z): 1023 [M+H]⁺

### Step 6. Synthesis of Compound 12-10

To a stirred mixture of tert-butyl N-[5-(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-1-oxo-3H-isoindol-4-yl)pent-4-yn-1-yl]carbamate (Compound 12-9, 300 mg, 0.29 mmol, 1.00 equiv) in DCM (3.00 mL) was added TFA (0.75 mL) dropwise at 0° C. The resulting mixture was stirred for 3 h at 25° C. LCMS indicated the reaction was completed. The resulting mixture was concentrated under reduced pressure. This resulted in N-{[({[(1S)-1-{[({[({3-[4-(5-aminopent-1-yn-1-yl)-1-oxo-3H-isoindol-2-yl]-2,6-dioxopiperidin-1-yl}methyl)carbamoyl]methyl}carbamoyl)methyl]carbamoyl}-2-phenylethyl]carbamoyl}methyl)carbamoyl]methyl}-6-(2,5-dioxopyrrol-1-yl)hexanamide; trifluoroacetic acid (Compound 12-10, 300 mg, 98%) as a yellow solid. LCMS (ES, m/z): 923 [M+H]⁺

### Step 7. Synthesis of Compound 12-12

To a stirred mixture of (3'S,4′R,5'S)-6″-chloro-4′-(3-chloro-2-fluorophenyl)-2″-oxo-1″H-dispiro[cyclohexane-1,2′-pyrrolidine-3′,3″-indole]-5′-carboxylic acid (Compound 12-11, prepared as described in J. Med. Chem. 2014, 57, 10486-10498300 mg, 0.64 mmol, 1.00 equiv) and methyl 4-aminobenzoate (117 mg, 0.77 mmol, 1.2 equiv) in DMA (8 mL) was added DIEA (100 mg, 0.77 mmol, 1.2 equiv) at 0° C. The resulting mixture was stirred for 30 min at 0° C. To the above mixture was added HATU (295 mg, 0.77 mmol, 1.2 equiv) at 0° C. The resulting mixture was stirred for additional 16 h at 25° C. LCMS indicated the reaction was completed. The reaction was quenched by the addition of water (0.5 mL) at room temperature. The residue was purified by reverse flash chromatography with the following conditions: column, C18 silica gel; mobile phase, water (0.05% TFA), ACN (5% to 50% gradient in 30 min; detector, UV 254 nm. This resulted in methyl 4-[(3'S,4′R,5'S)-6″-chloro-4′-(3-chloro-2-fluorophenyl)-2″-oxo-1″H-dispiro[cyclohexane-1,2′-pyrrolidine-3′,3″-indol]-5′-ylamido]benzoate (Compound 12-12, 200 mg, 51%) as a yellow solid. LCMS (ES, m/z): 596,598 [M+H]⁺

### Step 8. Synthesis of Compound 12-13

To a stirred mixture of methyl 4-[(3'S,4′R,5'S)-6″-chloro-4′-(3-chloro-2-fluorophenyl)-2″-oxo-1″H-dispiro[cyclohexane-1,2′-pyrrolidine-3′,3″-indol]-5′-ylamido]benzoate (Compound 12-12, 190 mg, 0.31 mmol, 1.00 equiv) in THE (2 mL) was added NaOH (12 mg, 0.31 mmol, 1 equiv) and LiOH (15 mg, 0.63 mmol, 2 equiv) in H₂O (2 mL) dropwise at 0° C. The resulting mixture was stirred for 4 h at 25° C. LCMS indicated the reaction was completed. The resulting mixture was concentrated under reduced pressure. The mixture was acidified to pH 6 with HCl (aq.). The residue was purified by reverse flash chromatography with the following conditions: column, C18 silica gel; mobile phase, water (0.05% TFA), ACN 5% to 50% gradient in 30 min; detector, UV 254 nm. This resulted in 4-[(3'S,4′R,5'S)-6″-chloro-4′-(3-chloro-2-fluorophenyl)-2″-oxo-1″H-dispiro[cyclohexane-1,2′-pyrrolidine-3′,3″-indol]-5′-ylamido]benzoic acid (Compound 12-13, 50 mg, 26%) as a yellow solid. LCMS (ES, m/z): 582,584 [M+H]⁺

### Step 9. Synthesis of Compound (XII)

To a stirred mixture of 4-[(3'S,4′R,5'S)-6″-chloro-4′-(3-chloro-2-fluorophenyl)-2″-oxo-1″H-dispiro[cyclohexane-1,2′-pyrrolidine-3′,3″-indol]-5′-ylamido]benzoic acid (Compound 12-13, 50 mg, 0.086 mmol, 1.00 equiv) in DMF (0.9 mL) was added HATU (36 mg, 0.095 mmol, 1.1 equiv) in portions at 0° C. The resulting mixture was stirred for 10 min at 0° C. To the above mixture was added N-{[({[(1S)-1-{[({[({3-[4-(5-aminopent-1-yn-1-yl)-1-oxo-3H-isoindol-2-yl]-2,6-dioxopiperidin-1-yl}methyl)carbamoyl]methyl}carbamoyl)methyl]carbamoyl}-2-phenylethyl]carbamoyl}methyl)carbamoyl]methyl}-6-(2,5-dioxopyrrol-1-yl)hexanamide; trifluoroacetic acid (Compound 12-10, 98 mg, 0.095 mmol, 1.10 equiv) and DIEA (33 mg, 0.25 mmol, 3 equiv) at 0° C. The resulting mixture was stirred for additional 16 h at 25° C. LCMS indicated the reaction was completed. The reaction was quenched by the addition of water (0.1 mL) at room temperature. The crude product was purified by Prep-HPLC with the following conditions Column: XBridge Shield RP18 OBD Column, 30×150 mm, 5 m; Mobile Phase A: water (0.1% FA), Mobile Phase B: ACN; Flow rate: 60 mL/min; Gradient: 37% B to 53% B in 10 min, 53% B to 53% B in 11 min; Wave Length: 254 nm; RT1 (min): 10.52. The collected fraction was lyophilized to afford (3′R,4'S,5′R)-6″-chloro-4′-(3-chloro-2-fluorophenyl)-N-(4-{[5-(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-1-oxo-3H-isoindol-4-yl)pent-4-yn-1-yl]carbamoyl}phenyl)-2″-oxo-1″H-dispiro[cyclohexane-1,2′-pyrrolidine-3′,3″-indole]-5′-carboxamide (Compound (XII), 24.1 mg, 18%) as a white solid. LCMS (ES, m/z): 1486, 1488 [M+H]^{+ 1}H-NMR (DMSO, 400 MHz) δ (ppm): 10.59 (s, 1H), 10.23 (s, 1H), 8.47-8.44 (m, 1H), 8.36-8.26 (m, 1H), 8.25-7.90 (m, 5H), 7.80 (d, J=8.8 Hz, 2H), 7.76-7.58 (m, 5H), 7.53-7.45 (m, 2H), 7.37-7.34 (m, 1H), 7.26-7.13 (m, 6H), 7.05-6.99 (m, 3H), 6.68 (d, J=2.0 Hz, 1H), 5.32-5.10 (m, 2H), 5.02-4.90 (m, 1H), 4.77-4.68 (m, 2H), 4.50-4.46 (m, 2H), 4.36-4.32 (m, 1H), 3.76-3.60 (m, 9H), 3.42-3.36 (m, 4H), 3.06-3.02 (m, 2H), 2.82-2.70 (m, 2H), 2.55-2.54 (m, 2H), 2.46-2.43 (m, 1H), 2.11-2.04 (m, 4H), 1.84-1.80 (m, 3H), 1.68-1.52 (m, 4H), 1.49-1.36 (m, 6H), 1.19-1.15 (m, 2H), 1.05-0.96 (m, 1H), 0.92-0.82 (m, 1H).

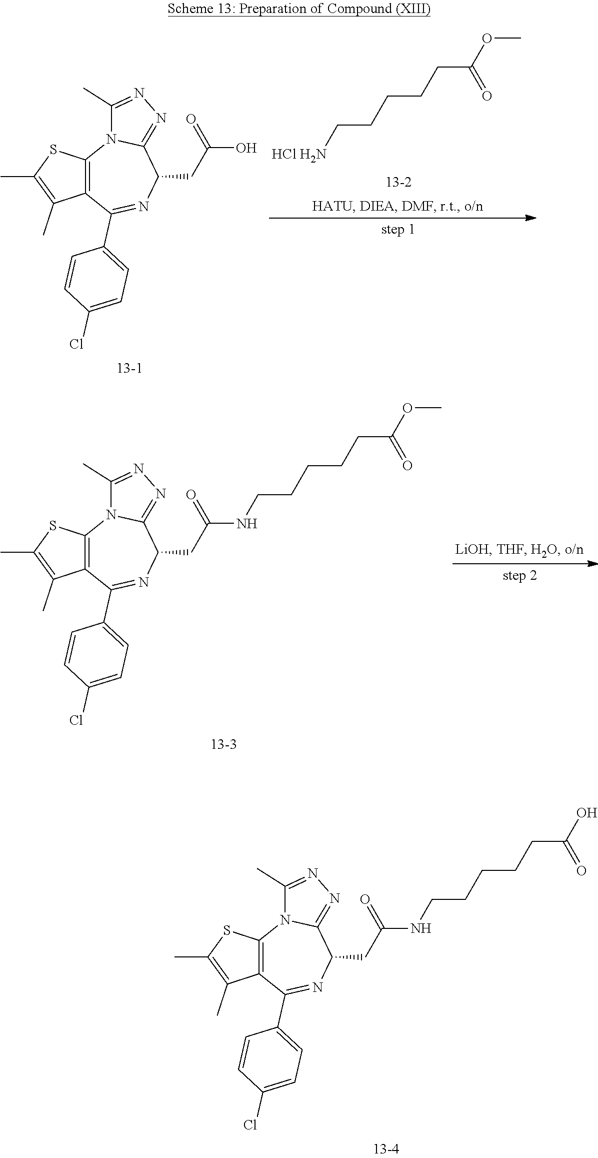

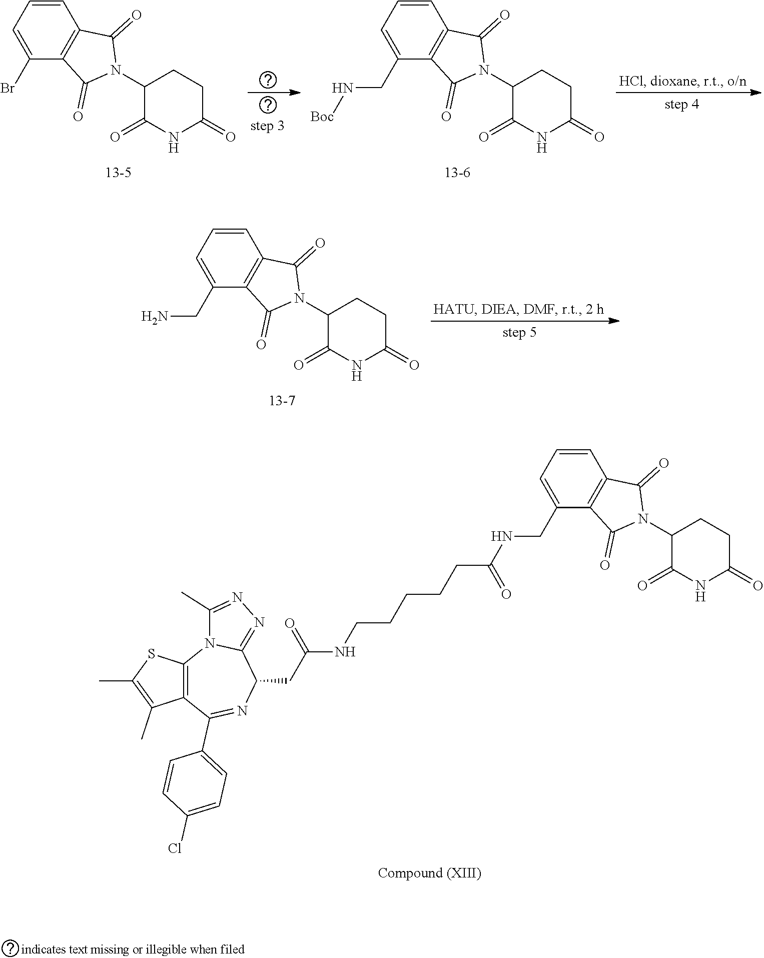 [SMILES: O=C1CCC(N2C(=O)c3cccc(Br)c3C2=O)C(=O)N1 ; CC(C)(C)OC(=O)NCc1cccc2c1C(=O)N(C1CCC(=O)NC1=O)C2=O ; NCc1cccc2c1C(=O)N(C1CCC(=O)NC1=O)C2=O ; Cc1sc2c(c1C)C(c1ccc(Cl)cc1)=N[C@@H](CC(=O)NCCCCCC(=O)NCc1cccc3c1C(=O)N(C1CCC(=O)NC1=O)C3=O)c1nnc(C)n1-2]

### Step 1. Synthesis of Compound 13-3

A solution of [(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetic acid (Compound 13-1, 1.00 g, 2.50 mmol, 1.00 equiv) and HATU (1.90 g, 4.99 mmol, 2.00 equiv) in DMF (10 mL) was stirred at room temperature for 0.5 h. Then methyl 6-aminohexanoate hydrochloride (Compound 13-2, 0.54 g, 2.994 mmol, 1.2 equiv) and DIEA (1.93 g, 14.97 mmol, 6.00 equiv) were added. The resulting mixture was stirred at room temperature for 2 hours. LCMS indicated the reaction was completed. The resulting mixture was diluted with water (100 mL), extracted with EA (50 mL×3), the combined organic layer was washed with water (50 mL), brine (50 mL), dried over anhydrous sodium sulfate and concentrated to dryness under vacuum. The residue was purified by silica gel column chromatography (DCM:MeOH=13:1) to give methyl 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanoate (Compound 13-3, 1.22 g, 91%) as a brown solid. LCMS (ES, m/z): 528,530 [M+H]⁺

### Step 2. Synthesis of Compound 13-4

To a solution of methyl 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanoate (Compound 13-3, 1.20 g, 2.27 mmol, 1.00 equiv) in THE (10 mL) and H₂O (5 mL) was added LiOH·H₂O (65 mg, 2.73 mmol, 1.20 equiv) at room temperature. The resulting mixture was stirred at room temperature for 2 hours. LCMS indicated the reaction was completed. The solvent was removed under vacuum. The residue was dissolved with EA (100 ml) and water (300 mL). The organic layer was separated out. The water phase was extracted with EA (100 mL×3). The combined organic layer was dried over anhydrous sodium sulfate and concentrated to dryness under vacuum to give 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanoic acid (Compound 13-4, 1 g, 84%) as a yellow solid. LCMS (ES, m/z): 514,516 [M+H]⁺

### Step 3. Synthesis of Compound 13-6

To a solution of IR[DF(CF₃)PPY]₂(DTBPY)PF₆ (166 mg, 0.15 mmol, 0.10 equiv), Nickel(2+), tetraaqua[4,4′-bis(1,1-dimethylethyl)-2,2′-bipyridine-κN1,κN1′]-chloride (69 mg, 0.15 mmol, 0.10 equiv), 4-bromo-2-(2,6-dioxopiperidin-3-yl)isoindole-1,3-dione (Compound 13-5, 500 mg, 1.48 mmol, 1.00 equiv) and [(tert-butoxycarbonyl)amino]acetic acid (390 mg, 2.22 mmol, 1.50 equiv) in DMSO (25.00 mL) was added 2-tert-Butyl-1,1,3,3-tetramethylguanidine (381 mg, 2.22 mmol, 1.50 equiv) at room temperature. The reaction vial was sealed and the reaction mixture sparged with nitrogen gas for 5 min. The stirring reaction mixture was then irradiated with blue LEDs (365 nm) for 24 hours. LCMS indicated the reaction was completed. The reaction was run twice in parallel. The reaction was diluted with water (250 mL), extracted with EA (75 mL×3), the combined organic layer was washed with water (75 mL), brine (75 mL), dried over anhydrous sodium sulfate and concentrated to dryness under vacuum. The residue was purified by reverse flash chromatography with the following conditions: column, C18 silica gel; mobile phase, ACN in water (0.1% FA), 0% to 60% gradient in 30 min; detector, UV 254 nm. The collected fraction was concentrated to give tert-butyl N-{[2-(2,6-dioxopiperidin-3-yl)-1,3-dioxoisoindol-4-yl]methyl}carbamate (Compound 13-6, 300 mg, 22%) as a yellow solid. LCMS (ES, m/z): 388 [M+H]⁺, 288 [M+H−Boc]⁺, 329 [M+H−Boc+ACN]⁺

### Step 4. Synthesis of Compound 13-7

A solution of tert-butyl N-{[2-(2,6-dioxopiperidin-3-yl)-1,3-dioxoisoindol-4-yl]methyl}carbamate (Compound 13-6, 300 mg, 0.77 mmol, 1.00 equiv) in HCl(gas) in 1,4-dioxane (15 mL) was stirred at room temperature overnight. LCMS indicated the reaction was completed. The reaction was concentrated to dryness under vacuum to give 4-(aminomethyl)-2-(2,6-dioxopiperidin-3-yl)isoindole-1,3-dione (Compound 13-7, 380 mg, crude) as a yellow solid. The crude product was used to next step without further purification. LCMS (ES, m/z): 288 [M+H]⁺

### Step 5. Synthesis of Compound (XIII)

To a solution of 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanoic acid (Compound 13-4, 390 mg, 0.76 mmol, 1.00 equiv) in DMF (5 mL) was added 4-(aminomethyl)-2-(2,6-dioxopiperidin-3-yl)isoindole-1,3-dione (Compound 13-7, 376 mg, 0.76 mmol, 1.00 equiv, 58%), HATU (577 mg, 1.52 mmol, 2.00 equiv), DIEA (294 mg, 2.28 mmol, 3.00 equiv) at room temperature in air. The resulting mixture was stirred at room temperature for 2 hours. LCMS indicated the reaction was completed. The resulting mixture was diluted with water (50 mL), extracted with EA (50 mL×3), the combined organic layer was washed with brine (50 mL), water (50 mL), dried over anhydrous sodium sulfate and concentrated to dryness under vacuum. The residue was purified by Prep-TLC (DCM:MeOH=10:1) to give 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}-N-{[2-(2,6-dioxopiperidin-3-yl)-1,3-dioxoisoindol-4-yl]methyl}hexanamide (Compound (XIII), 330 mg, 52%) as a yellow solid. MS: (ES, m/s): 783,785 [M+H]^{+ 1}H NMR (400 MHz, DMSO-d₆) δ11.14 (s, 1H), 8.45 (t, J=5.6 Hz, 1H), 8.18 (t, J=4.4 Hz, 1H), 7.85-7.77 (m, 2H), 7.67 (d, J=7.6 Hz, 1H), 7.50-7.40 (dd, J=8.8 Hz, 20.8 Hz, 4H), 5.22-5.16 (m, 1H), 4.71-4.72 (m, 2H), 4.53-4.49 (m, 1H), 3.30-3.06 (m, 4H), 2.95-2.87 (m, 1H), 2.67-2.51 (m, 5H), 2.41 (s, 1H), 2.20 (t, J=7.2 Hz, 2H), 2.08-2.04 (m, 2H), 1.62 (s, 1H), 1.53-1.59 (m, 2H), 1.48-1.42 (m, 2H), 1.35-1.29 (m, 2H).

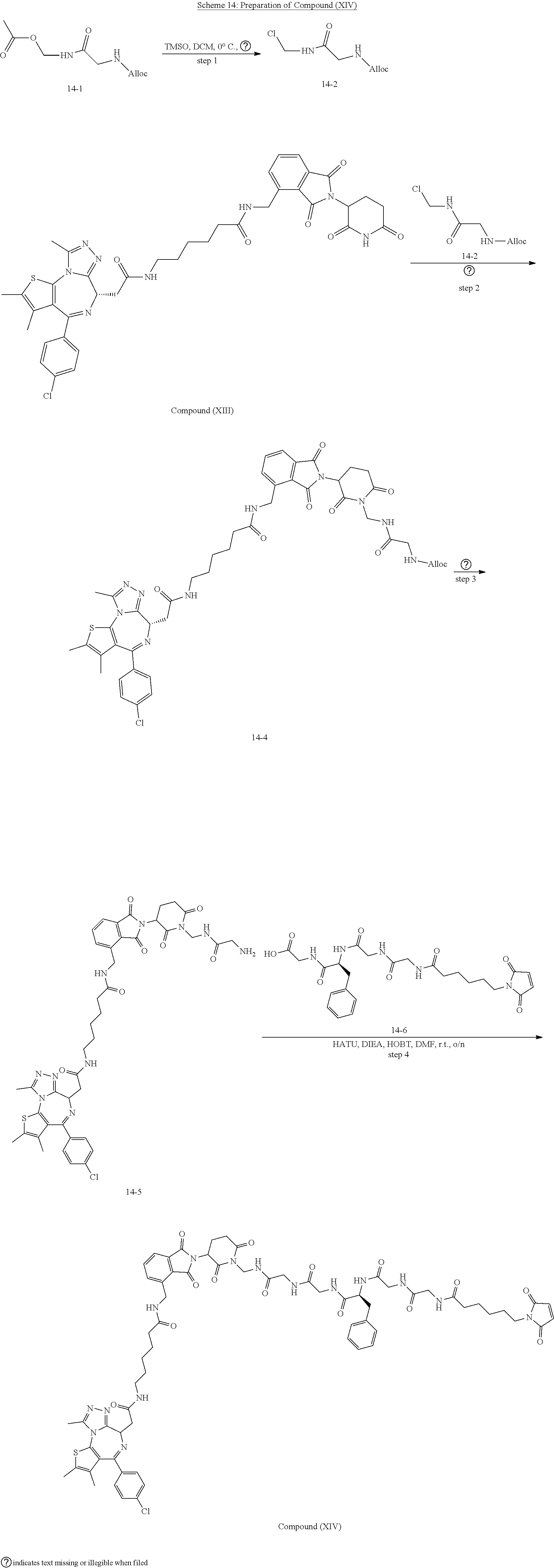

### Step 1. Synthesis of Compound 14-2

To a solution of (2-{[(prop-2-en-1-yloxy)carbonyl]amino}acetamido)-methyl acetate (Compound 14-1, prepared according to the procedure described for Compound 10-3, 100 mg. 0.44 mmol. 1.00 equiv) in DCM (10 mL) was added TMSCl (70 mg. 0.66 mmol. 1.50 equiv) at room temperature. The reaction was stirred at room temperature for 0.5 h. LCMS indicated the reaction was completed. The reaction was concentrated to dryness under vacuum and the residue was used directly to the next step. LCMS (ES, m/z): 203 [M+H]⁺ (derivated with methanol)

### Step 2. Synthesis of Compound 14-4

To a solution of 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}-N-{[2-(2,6-dioxopiperidin-3-yl)-1,3-dioxoisoindol-4-yl]methyl}hexanamide (Compound (XIII), 100 mg, 0.12 mmol, 1.00 equiv) and prop-2-en-1-yl N-[(chloromethylcarbamoyl)methyl]carbamate (Compound 14-3, 106 mg, 0.52 mmol, 4.00 equiv) in DMF (5 mL) was added K₂CO₃ (70 mg, 0.52 mmol, 4.00 equiv) at room temperature. The resulting mixture was stirred at room temperature for 48 hours. LCMS indicated the reaction was completed. The reaction mixture was purified by reverse flash chromatography with the following conditions: column, C18 silica gel; mobile phase, ACN in water (0.1% FA), 0% to 60% gradient in 30 min; detector, UV 254 nm&220 nm. The collected fraction was concentrated to give prop-2-en-1-yl N-({[(3-{4-[(6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanamido)methyl]-1,3-dioxoisoindol-2-yl}-2,6-dioxopiperidin-1-yl)methyl]carbamoyl}methyl)carbamate (Compound 14-4, 110 mg, 83%) as a yellow solid. LCMS (ES, m/z): 953,955 [M+H]⁺

### Step 3. Synthesis of Compound 14-5

To a solution of prop-2-en-1-yl N-({[(3-{4-[(6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanamido)methyl]-1,3-dioxoisoindol-2-yl}-2,6-dioxopiperidin-1-yl)methyl]carbamoyl}methyl)carbamate (Compound 14-4, 100 mg, 0.11 mmol, 1.00 equiv) in THE (5 mL) were added phenylsilane (23 mg, 0.21 mmol, 2.00 equiv) and Pd(PPh₃)₄ (12 mg, 0.01 mmol, 0.10 equiv) under N₂. The resulting mixture was stirred at room temperature for 3 hours. LCMS indicated the reaction was completed. The resulting mixture was purified by reverse flash chromatography with the following conditions: column, C18 silica gel; mobile phase, ACN in water (0.1% FA), 0% to 60% gradient in 30 min; detector, UV 254 nm & 220 nm to give (Compound 14-5, 55 mg, 57%) as a pale yellow solid. LCMS (ES, m/z): 869,871 [M+H]⁺

### Step 4. Synthesis of Compound (XIV)

To a solution of N-[(2-{1-[(2-aminoacetamido)methyl]-2,6-dioxopiperidin-3-yl}-1,3-dioxoisoindol-4-yl)methyl]-6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}hexanamide (Compound 14-5, 50 mg, 0.06 mmol, 1.00 equiv), [(2S)-2-(2-{2-[6-(2,5-dioxopyrrol-1-yl)hexanamido]acetamido}acetamido)-3-phenylpropanamido]acetic acid (Compound 14-4, prepared according to the procedure described for Compound 8-12, 34 mg, 0.06 mmol, 1.10 equiv) in DMF (3 mL) were added HOBT (16 mg, 0.12 mmol, 2.00 equiv), HATU (44 mg, 0.12 mmol, 2.00 equiv) and DIEA (67 mg, 0.52 mmol, 9.00 equiv) at room temperature. The resulting mixture was stirred at room temperature overnight. The resulting mixture was purified by Column: XSelect CSH Prep C18 OBD Column, 19*150 mm, 5 m; Mobile Phase A: Water (0.05% FA), Mobile Phase B: ACN; Flow rate: 25 mL/min; Gradient: 29% B to 59% B in 7 min, 59% B; Wave Length: 254 nm; RT1 (min): 6.8 to give 6-{2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-yl]acetamido}-N-[(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-1,3-dioxoisoindol-4-yl)methyl]hexanamide (Compound (XIV), 14.4 mg, 17%) as a light yellow solid. LCMS (ES, m/z): 1380,1382 [M+H]¹H NMR (400 MHz, DMSO-d₆) δ8.47-8.44 (m, 1H), 8.29-8.21 (m, 2H), 8.19-8.15 (m, 1H), 8.13-8.04 (m, 2H), 8.00-7.92 (m, 2H), 7.89-7.72 (m, 2H), 7.68-7.66 (m, 1H), 7.50-7.41 (m, 4H), 7.35-7.23 (m, 4H), 7.20-7.11 (m, 1H), 6.99 (s, 2H), 5.25-5.12 (m, 2H), 5.08-5.00 (m, 1H), 4.72 (d, J=6 Hz, 2H), 4.53-4.50 (m, 2H), 3.76-3.69 (m, 5H), 3.68-3.65 (m, 4H), 3.36 (t, J=7.2 Hz, 2H), 3.24-3.20 (m, 2H), 3.10-3.03 (m, 4H), 2.82-2.78 (m, 2H), 2.60 (s, 3H), 2.41 (s, 3H), 2.23-2.19 (m, 2H), 2.19-2.01 (m, 3H), 1.62 (s, 3H), 1.59-1.55 (m, 2H), 1.50-1.42 (m, 6H), 1.33-1.31 (m, 2H), 1.20-1.16 (m, 2H).

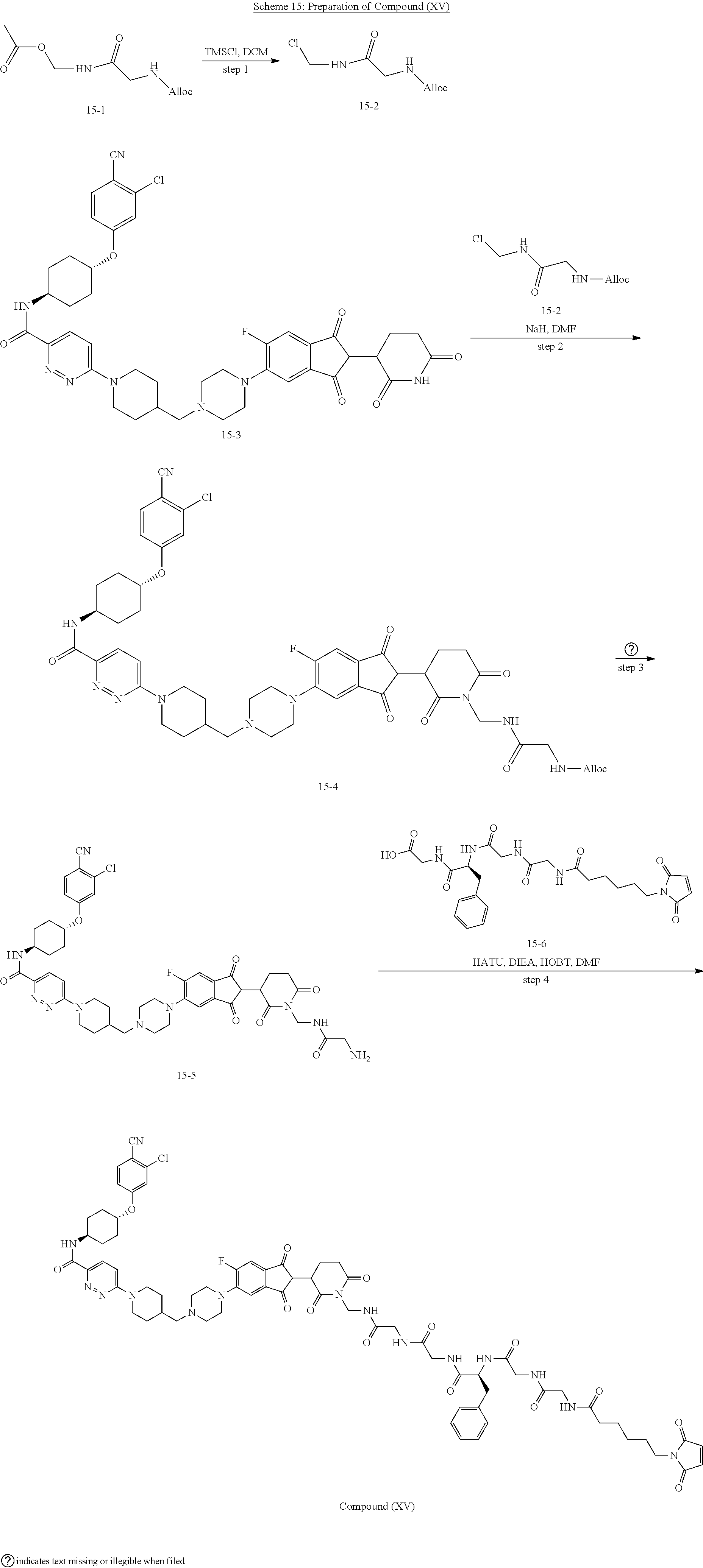

### Step 1. Synthesis of Compound 15-2

To a stirred mixture of (2-{[(prop-2-en-1-yloxy) carbonyl]amino}acetamido)methyl acetate (Compound 15-1, prepared according to the procedure described for Compound 10-3, 736 mg 3.19 mmol, 1 equiv) in DCM (30 mL) was added TMSCl (1389 mg, 12.78 mmol, 4.00 equiv) dropwise at 0° C. The resulting mixture was stirred for 2 h at 0° C. LCMS indicated the reaction was completed. The resulting mixture was concentrated under reduced pressure. This resulted in prop-2-en-1-yl N-[(chloromethylcarbamoyl)methyl]carbamate (Compound 15-2, 736 mg, 89%) as a white solid. LCMS (ES, m/z): 203 [M+H]⁺ (derivated with MeOH).

### Step 2. Synthesis of Compound 15-4

To a stirred mixture of 6-[4-({4-[2-(2,6-dioxopiperidin-3-yl)-6-fluoro-1,3-dioxoisoindol-5-yl]piperazin-1-yl}methyl)piperidin-1-yl]-N-[(1r,4r)-4-(3-chloro-4-cyanophenoxy)cyclohexyl]pyridazine-3-carboxamide (Compound 15-3, 500 mg, 0.61 mmol, 1 equiv) in DMF (8 mL) was added NaH (37 mg, 0.92 mmol, 1.50 equiv, 60%) in portions at 0° C. under nitrogen atmosphere. The resulting mixture was stirred for 30 min at 0° C. under nitrogen atmosphere. To the above mixture was added prop-2-en-1-yl N-[(chloromethylcarbamoyl)methyl]carbamate (Compound 15-2, 254 mg, 1.23 mmol, 2 equiv) at 0° C. The resulting mixture was stirred for additional 16 h at 25° C. LCMS indicated the reaction was completed. The reaction was quenched by the addition of water (1 mL) at room temperature. The residue was purified by reverse flash chromatography with the following conditions: column, C18 silica gel; mobile phase, water (0.05% TFA), ACN 5% to 50% gradient in 30 min; detector, UV 254 nm. This resulted in prop-2-en-1-yl N-{[({3-[5-fluoro-1,3-dioxo-6-(4-{[1-(6-{[(1r,4r)-4-(3-chloro-4-cyanophenoxy)cyclohexyl]carbamoyl}pyridazin-3-yl)piperidin-4-yl]methyl}piperazin-1-yl)isoindol-2-yl]-2,6-dioxopiperidin-1-yl}methyl)carbamoyl]methyl}carbamate (Compound 15-4, 130 mg, 21%) as a yellow solid. LCMS (ES, m/z): 982,984 [M+H]⁺

### Step 3. Synthesis of Compound 15-5

To a stirred mixture of prop-2-en-1-yl N-{[({3-[5-fluoro-1,3-dioxo-6-(4-{[1-(6-{[(1r,4r)-4-(3-chloro-4-cyanophenoxy)cyclohexyl]carbamoyl}pyridazin-3-yl)piperidin-4-yl]methyl}piperazin-1-yl)isoindol-2-yl]-2,6-dioxopiperidin-1-yl}methyl)carbamoyl]methyl}carbamate (Compound 15-4, 120 mg, 0.12 mmol, 1 equiv) and Pd(PPh₃)₄ (14 mg, 0.012 mmol, 0.1 equiv) in THF (1.2 mL) was added phenylsilane (26 mg, 0.24 mmol, 2 equiv) dropwise at room temperature under nitrogen atmosphere. The resulting mixture was stirred for 3 h at 25° C. under nitrogen atmosphere. LCMS indicated the reaction was completed. The resulting mixture was concentrated under reduced pressure. The residue was purified by reverse flash chromatography with the following conditions: column, C18 silica gel; mobile phase, water (0.05% TFA), ACN 5% to 50% gradient in 30 min; detector, UV 254 nm. This resulted in 6-(4-{[4-(2-{1-[(2-aminoacetamido)methyl]-2,6-dioxopiperidin-3-yl}-6-fluoro-1,3-dioxoisoindol-5-yl)piperazin-1-yl]methyl}piperidin-1-yl)-N-[(1r,4r)-4-(3-chloro-4-cyanophenoxy)cyclohexyl]pyridazine-3-carboxamide (Compound 15-5, 40 mg, 36%) as a yellow solid. LCMS (ES, m/z): 899,901 [M+H]⁺

### Step 4. Synthesis of Compound (XV)

To a stirred mixture of [(2S)-2-(2-{2-[6-(2,5-dioxopyrrol-1-yl)hexanamido]acetamido}acetamido)-3-phenylpropanamido]acetic acid (Compound 15-6, prepared according to the procedure described for Compound 8-12, 21 mg, 0.040 mmol, 1 equiv) and HATU (18 mg, 0.048 mmol, 1.2 equiv), HOBT (7 mg, 0.048 mmol, 1.2 equiv) in DMF (0.4 mL) was added 6-(4-{[4-(2-{1-[(2-aminoacetamido)methyl]-2,6-dioxopiperidin-3-yl}-6-fluoro-1,3-dioxoisoindol-5-yl)piperazin-1-yl]methyl}piperidin-1-yl)-N-[(1r,4r)-4-(3-chloro-4-cyanophenoxy)cyclohexyl]pyridazine-3-carboxamide (Compound 15-5, 40 mg, 0.040 mmol, 1 equiv) and DIEA (16 mg, 0.12 mmol, 3 equiv) at 0° C. under nitrogen atmosphere. The resulting mixture was stirred for 16 h at 25° C. under nitrogen atmosphere. LCMS indicated the reaction was completed. The crude product was purified by Prep-HPLC with the following conditions Column: XBridge Shield RP18 OBD Column, 30×150 mm, 5 m; Mobile Phase A: water (0.1% FA), Mobile Phase B: ACN; Flow rate: 60 mL/min; Gradient: 23% B to 43% B in 10 min, 43% B; Wave Length: 254 nm; RT1 (min): 8.92; The collected fraction was lyophilized to afford 6-(4-{[4-(2-{1-[(2-{2-[(2S)-2-(2-{2-[6-(2,5-dioxopyrrol-1-yl)hexanamido]acetamido}acetamido)-3-phenylpropanamido]acetamido}acetamido)methyl]-2,6-dioxopiperidin-3-yl}-6-fluoro-1,3-dioxoisoindol-5-yl)piperazin-1-yl]methyl}piperidin-1-yl)-N-[(1r,4r)-4-(3-chloro-4-cyanophenoxy)cyclohexyl]pyridazine-3-carboxamide (Compound (XV), 7.2 mg, 10%) as a yellow solid. LCMS (ES, m/z): 1409, 1411 [M+H]⁺. ¹H-NMR (DMSO-d₆, 400 MHz) δ (ppm): 8.59 (d, J=8.0 Hz, 1H), 8.28-8.24 (m, 2H), 8.13-8.06 (m, 2H), 7.99-7.92 (m, 2H), 7.86-7.73 (m, 3H), 7.39-7.31 (m, 3H), 7.24-7.21 (m, 4H), 7.17-7.12 (m, 2H), 6.99 (s, 2H), 5.17-5.14 (m, 3H), 4.53-4.47 (m, 4H), 3.74-3.50 (m, 11H), 3.37-3.59 (m, 2H), 3.30-3.22 (m, 3H), 3.07-2.76 (m, 7H), 2.62-2.52 (m, 3H), 2.32-2.00 (m, 7H), 1.91-1.83 (m, 5H), 1.65-1.62 (m, 2H), 1.52-1.43 (m, 6H), 1.20-1.14 (m, 4H).

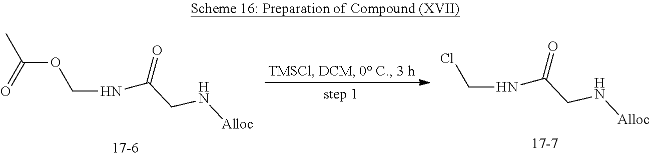

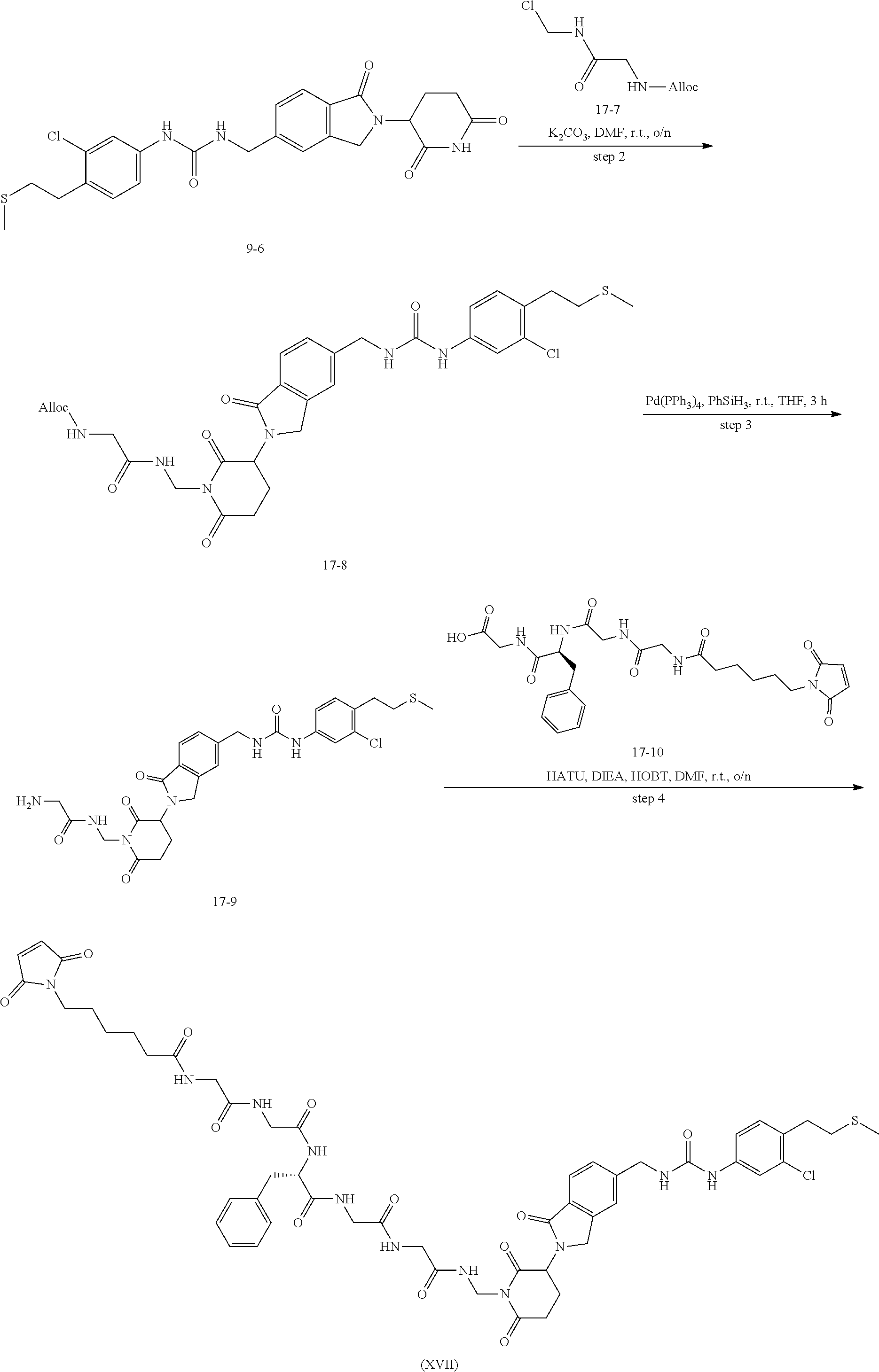

### Step 1. Synthesis of Compound 17-7

To a solution of (2-{[(prop-2-en-1-yloxy)carbonyl]amino}acetamido)methyl acetate (Compound 17-6, 100 mg, 0.43 mmol, 1.00 equiv) in DCM (5 mL) was added TMSCl (71 mg, 0.65 mmol, 1.50 equiv) at room temperature. The reaction was stirred at room temperature for 0.5 h. LCMS indicated the reaction was completed. The reaction was concentrated to dryness under vacuum and the residue was used directly to the next step. LCMS (ES, m/z): 203 [M+H]⁺ (derivated with methanol)

### Step 2. Synthesis of Compound 17-8

A solution of 1-{3-chloro-4-[2-(methylsulfanyl)ethyl]phenyl}-3-{[2-(2,6-dioxopiperidin-3-yl)-1-oxo-3H-isoindol-5-yl]methyl}urea (Compound 9-6, 50 mg, 0.100 mmol, 1.00 equiv), prop-2-en-1-yl N-[(chloromethylcarbamoyl)methyl]carbamate (Compound 17-7, 82 mg, 0.40 mmol, 4.00 equiv) and K₂CO₃ (41 mg, 0.30 mmol, 3.00 equiv) in NMP (2500 uL) was stirred at 50° C. for 24 hours. Then prop-2-en-1-yl N-[(chloromethylcarbamoyl)methyl]carbamate (82 mg, 0.40 mmol, 4.00 equiv) and K₂CO₃ (14 mg, 0.10 mmol, 1.00 equiv) in NMP (0.5 mL) was added. The resulting mixture was stirred at 50° C. for 24 hours. LCMS indicated the reaction was completed. The reaction was run by eight times in parallel. The resulting mixture was purified by Prep-HPLC with the following condition: Column: Sunfire Prep C18 OBD Column, 19*250 mm, 10 m; Mobile Phase A: Water (0.1% FA), Mobile Phase B: ACN; Flow rate: 25 mL/min; Gradient: 41% B to 57% B in 10 min, 57% B; Wave Length: 254 nm; RT1 (min) to give prop-2-en-1-yl N-[({[3-(5-{[({3-chloro-4-[2-(methylsulfanyl)ethyl]phenyl}carbamoyl)amino]methyl}-1-oxo-3H-isoindol-2-yl)-2,6-dioxopiperidin-1-yl]methyl}carbamoyl)methyl]carbamate (Compound 17-8, 64 mg, 11%) of the product as a yellow solid. LCMS (ES, m/z): 671 [M+H]⁺

### Step 3. Synthesis of Compound 17-9

To a solution of prop-2-en-1-yl N-[({[3-(5-{[({3-chloro-4-[2-(methylsulfanyl)ethyl]phenyl}carbamoyl)amino]methyl}-1-oxo-3H-isoindol-2-yl)-2,6-dioxopiperidin-1-yl]methyl}carbamoyl)methyl]carbamate (Compound 17-8, 64 mg, 0.10 mmol, 1.00 equiv) in THF (5 mL) and phenylsilane (21 mg, 0.19 mmol, 2.00 equiv) was added Pd(PPh₃)₄ (11 mg, 0.01 mmol, 0.10 equiv) under N₂. The resulting mixture was stirred at room temperature for 3 hours. LCMS indicated the reaction was completed. After filtration, the reaction mixture was purified by reverse flash chromatography with the following conditions: column, C18 silica gel; mobile phase, ACN in water (0.1% FA), 0% to 60% gradient in 30 min; detector, UV 254 nm. The collected fraction was lyophilized to give 2-amino-N-{[3-(5-{[({3-chloro-4-[2-(methylsulfanyl)ethyl]phenyl}carbamoyl)amino]methyl}-1-oxo-3H-isoindol-2-yl)-2,6-dioxopiperidin-1-yl]methyl}acetamide (Compound 17-9, 32 mg, 51%) as an off-white solid. LCMS (ES, m/z): 587 [M+H]⁺

### Step 4. Synthesis of Compound XVII

A solution of [(2S)-2-(2-{2-[6-(2,5-dioxopyrrol-1-yl)hexanamido]acetamido}acetamido)-3-phenylpropanamido]acetic acid (Compound 17-9, 32 mg, 0.06 mmol, 1.20 equiv), HOBT (7 mg, 0.05 mmol, 1.00 equiv) and HATU (19 mg, 0.05 mmol, 1.00 equiv) in DMF (3 mL) was stirred at room temperature for 1 h. Then 2-amino-N-{[3-(5-{[({3-chloro-4-[2-(methylsulfanyl)ethyl]phenyl}carbamoyl)amino]methyl}-1-oxo-3H-isoindol-2-yl)-2,6-dioxopiperidin-1-yl]methyl}acetamide (Compound 17-10, prepared according to the procedure described for Compound 8-12, 30 mg, 0.05 mmol, 1.00 equiv) and DIEA (26 mg, 0.20 mmol, 4.00 equiv) was added at room temperature. The resulting mixture was stirred at room temperature for overnight. LCMS indicated the reaction was completed. The reaction was purified by reverse flash chromatography with the following conditions: Column: Kinetex EVO C18, 21.2*250 mm, 5 m; Mobile Phase A: Water (0.1% FA), Mobile Phase B: ACN; Flow rate: 25 mL/min; Gradient: 34% B to 64% B in 7 min, 64% B; Wave Length: 254 nm; RT1 (min): 6. The collected fraction was lyophilized to give N-{[({[(1S)-1-[({[({[3-(5-{[({3-chloro-4-[2-(methylsulfanyl)ethyl]phenyl}carbamoyl)amino]methyl}-1-oxo-3H-isoindol-2-yl)-2,6-dioxopiperidin-1-yl]methyl}carbamoyl)methyl]carbamoyl}methyl)carbamoyl]-2-phenylethyl]carbamoyl}methyl)carbamoyl]methyl}-6-(2,5-dioxopyrrol-1-yl)hexanamide (Compound (XVII), 6.5 mg, 10.57%) as a white solid. LCMS (ES, m/z): 1098 [M+H]^{+ 1}H NMR (400 MHz, DMSO-d₆) δ 8.80 (s, 1H), 8.35-8.28 (m, 1H), 8.19-8.17 (m, 1H), 8.12-8.10 (m, 1H), 8.07-8.06 (m, 1H), 8.02-7.98 (m, 1H), 7.97-7.92 (m, 1H), 7.73-7.67 (m, 2H), 7.53 (s, 1H), 7.47-7.45 (m, 1H), 7.24-7.22 (m, 5H), 7.20-7.15 (m, 2H), 7.00 (s, 2H), 6.84-6.80 (m, 1H), 5.22-4.96 (m, 3H), 4.55-4.50 (m, 1H), 4.44-4.41 (m, 2H), 4.34-4.28 (m, 1H), 3.73-3.66 (m, 7H), 3.37-3.36 (m, 4H), 3.10-2.95 (m, 2H), 2.90-2.70 (m, 4H), 2.69-2.62 (m, 3H), 2.15-2.00 (m, 5H), 2.09-2.00 (m, 1H), 1.50-1.45 (m, 4H), 1.21-1.19 (m, 2H).

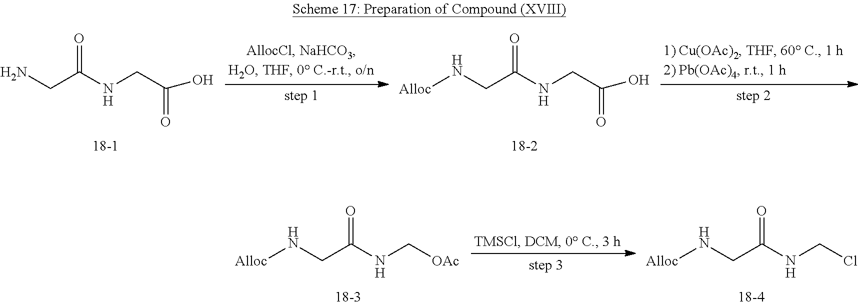

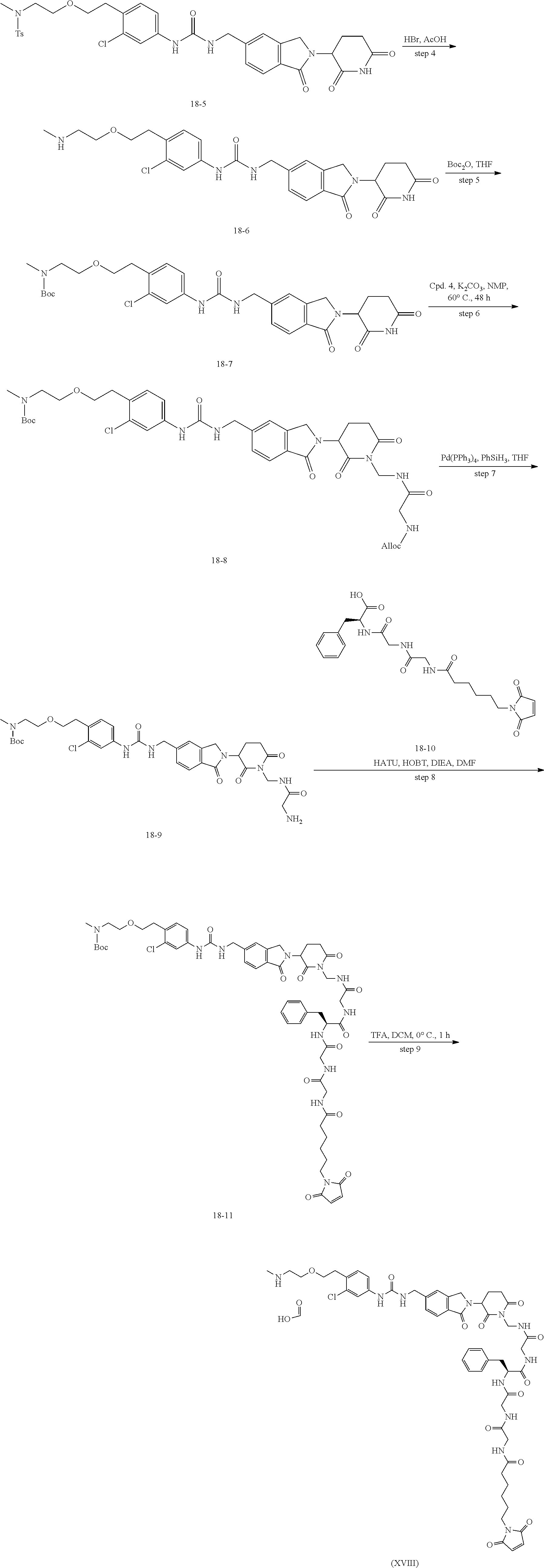

### Step 1. Synthesis of Compound 18-2

To a stirred mixture of Gly-Gly (10 g, 75.68 mmol, 1.00 equiv) and NaHCO₃ (12.72 g, 151.37 mmol, 2 equiv) in H₂O (70 mL) was added chloro(prop-2-en-1-yloxy)methanone (10.95 g, 90.82 mmol, 1.2 equiv) in THE (35 mL) dropwise at 0 degrees C. The resulting mixture was stirred for 5 h at 25 degrees C. LCMS indicated the reaction was completed. The resulting mixture was concentrated under reduced pressure. The mixture was acidified to pH 5 with HCl (aq. 1 N). The precipitated solids were collected by filtration and washed with HCl (aq. 1 N) (2×50 mL). This resulted in (2-{[(prop-2-en-1-yloxy)carbonyl]amino}acetamido)acetic acid (Compound 18-2, 9 g, 55%) as a white solid. LCMS (ES, m/z): 217 [M+H]⁺.

### Step 2. Synthesis of Compound 18-3

A mixture of (2-{[(prop-2-en-1-yloxy)carbonyl]amino}acetamido)acetic acid (Compound 18-2, 9 g, 41.62 mmol, 1.00 equiv) and Cu(OAc)₂ (0.76 g, 4.16 mmol, 0.1 equiv) in THE (220 mL) was stirred for 1 h at 60 degrees C. under nitrogen atmosphere. The mixture was allowed to cool down to room temperature. To the above mixture was added Pb(OAc)₄ (22.15 g, 49.95 mmol, 1.2 equiv) at room temperature. The resulting mixture was stirred for additional 1 h at 25 degrees C. LCMS indicated the reaction was completed. The resulting mixture was filtered, the filter cake was washed with MeOH (3×50 mL). The filtrate was concentrated under reduced pressure. The residue was purified by silica gel column chromatography, eluted with PE/EA (1:10) to afford (2-{[(prop-2-en-1-yloxy)carbonyl]amino}acetamido)methyl acetate (Compound 18-3, 5 g, 52%) as a white solid. LCMS: (ES, m/z): 253 [M+Na]⁺.

### Step 3. Synthesis of Compound 18-4

To a stirred solution of (2-{[(prop-2-en-1-yloxy)carbonyl]amino}acetamido)methyl acetate (Compound 18-3, 100 mg, 0.43 mmol, 1 equiv) in DCM (1 mL) was added TMSCl (188 mg, 1.73 mmol, 4 equiv) dropwise at 0° C. under nitrogen atmosphere. The resulting mixture was stirred for 2 h at 0° C. under nitrogen atmosphere. The reaction mixture was derivative with methanol for LCMS test. The resulting mixture was concentrated under vacuum. to afford crude product prop-2-en-1-yl N-[(chloromethylcarbamoyl)methyl]carbamate (Compound 18-4, 80 mg, 89%) as a white solid. LCMS (ESI, m/z): 203 [M+H]⁺ (derivative with methanol) Synthesis of Compound 18-5

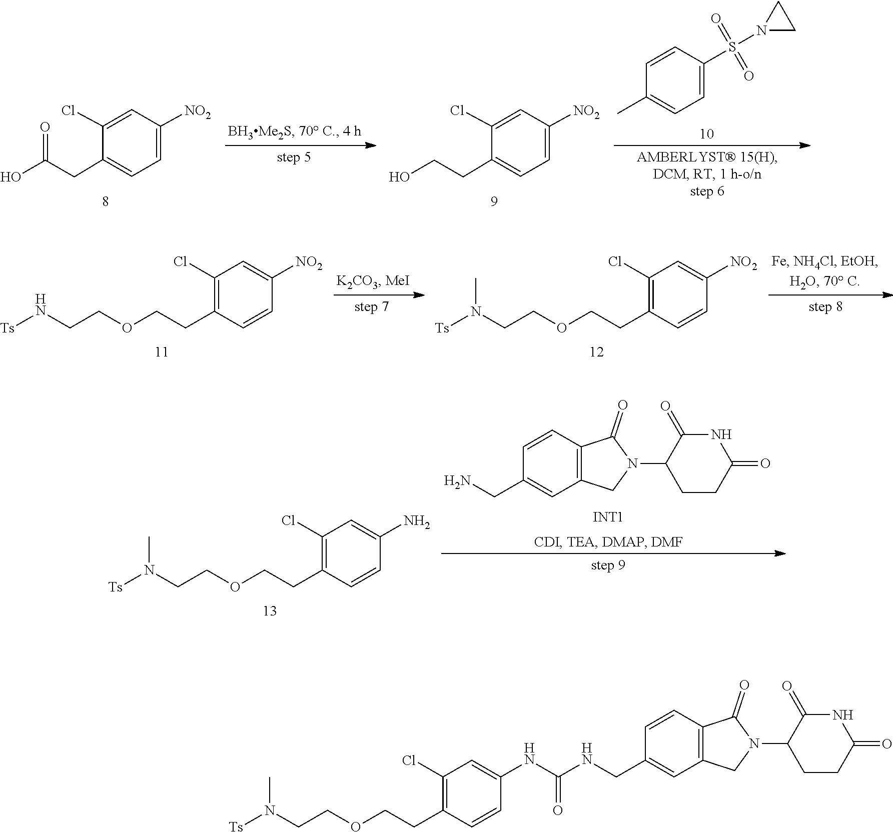

### Step 5

To a stirred solution of (2-chloro-4-nitrophenyl)acetic acid (40.7 g, 188.78 mmol, 1.00 equiv) in THE (610 mL) was added BH₃-Me₂S (47 mL, ION, 470 mmol, 2.5 equiv) dropwise at room temperature. The resulting mixture was stirred for 3 h at 70 degrees C. under nitrogen atmosphere. LCMS indicated the reaction was completed. The reaction mixture was allowed to room temperature and concentrated under reduced pressure. The residue was purified by silica gel column chromatography, eluted with PE/EA (1:1) to afford 2-(2-chloro-4-nitrophenyl)ethanol (35 g, 91%) as a yellow solid. ¹H NMR (300 MHz, DMSO-d₆) δ 8.25 (s, 1H), 8.16-8.12 (m, 1H), 7.68-7.65 (m, 1H), 4.87-4.84 (m, 1H), 3.71-3.65 (m, 2H), 2.9-2.95 (m, 2H).

### Step 6

To a stirred mixture of 2-(2-chloro-4-nitrophenyl)ethanol (6.0 g, 29.76 mmol, 1 equiv) and 1-(4-methylbenzenesulfonyl)aziridine (5.87 g, 29.75 mmol, 1.0 equiv) in DCM (60 mL) at room temperature under nitrogen atmosphere. The resulting mixture was stirred for 10 min at 0° C. To the above mixture was added AMBERLYST 15(H) (12.0 g) in portions at 0° C. The resulting mixture was stirred for additional 2 h at room temperature. LCMS indicated the reaction was 45% product. To the above mixture was added 1-(4-methylbenzenesulfonyl)aziridine (8.81 g, 44.64 mmol, 1.5 equiv) in portions at 0° C. The resulting mixture was stirred for additional 10 min at ° C. To the above mixture was added AMBERLYST 15(H) (12.0 g) in portions at 0° C. The resulting mixture was stirred for additional 2 h at room temperature. LCMS indicated the reaction was completed. The solids were filtered by filtration and washed with DCM (3×50 mL). The organic was concentrated to dryness under vacuum. The residue was purified by silica gel column chromatography, eluted with PE/EA (1:1) to afford N-{2-[2-(2-chloro-4-nitrophenyl)ethoxy]ethyl}-4-methylbenzenesulfonamide (6.8 g, 57%) as an off-white oil. LCMS: (ES·m/z): 399,401 [M+1]⁺. ¹HNMR (300 MHz, DMSO-d₆) δ 8.25 (s, 1H), 8.14-8.10 (m, 1H), 7.68-7.59 (m, 4H), 7.39-7.36 (m, 2H), 3.61-3.57 (m, 2H), 3.41-3.33 (m, 2H), 3.00-2.97 (m, 2H), 2.89-2.85 (m, 2H), 2.37 (s, 3H).

### Step 7

To a stirred mixture of N-{2-[2-(2-chloro-4-nitrophenyl)ethoxy]ethyl}-4-methylbenzenesulfonamide (6.8 g, 17.04 mmol, 1.00 equiv) in DMF (68 mL) was added K₂CO₃ (4.71 g, 34.08 mmol, 2.00 equiv) in portions at room temperature. The resulting mixture was stirred for 10 min at room temperature. To the above mixture was added MeI (3.7 g, 26.06 mmol, 1.53 equiv) dropwise at room temperature. The resulting mixture was stirred for overnight at room temperature. LCMS indicated the reaction was completed. The resulting mixture was diluted with water (132 mL) and extracted with EtOAc (3×150 mL). The combined organic layers were washed with water and brine (150 mL), dried over anhydrous Na₂SO₄. After filtration, the filtrate was concentrated under reduced pressure. The residue was purified by silica gel column chromatography, eluted with PE/EA (1:1) to afford N-{2-[2-(2-chloro-4-nitrophenyl)ethoxy]ethyl}-N,4-dimethylbenzenesulfonamide (7.3 g, crude) as a yellow oil. LCMS: (ES·m/z): 413,415 [M+1]⁺.

### Step 8

To a stirred mixture of N-{2-[2-(2-chloro-4-nitrophenyl)ethoxy]ethyl}-N,4-dimethylbenzenesulfonamide (7.3 g, crude) in EtOH (133 mL) was added Fe (5.55 g, 99.30 mmol) in portions and NH₄Cl (3.19 g, 59.58 mmol) in H₂O (27 mL) at room temperature. The resulting mixture was stirred for 4 h at 80° C. The mixture was allowed to cool down to room temperature. LCMS indicated the reaction was completed. The resulting mixture was filtered, the filter cake was washed with EtOH (3×20 mL). The filtrate was concentrated under reduced pressure. The residue was purified by silica gel column chromatography, eluted with PE/EA (1:1) to afford N-{2-[2-(4-amino-2-chlorophenyl)ethoxy]ethyl}-N,4-dimethylbenzenesulfonamide (4.4 g, 57% for two steps) as a yellow solid. LCMS: (ES·m/z): 382,384 [M+1]⁺.

### Step 9

To a stirred mixture of 3-[5-(aminomethyl)-1-oxo-3H-isoindol-2-yl]piperidine-2,6-dione (3.14 g, 11.49 mmol, 1.1 equiv) in DMF (48 mL) was added CDI (1.86 g, 11.49 mmol, 1.1 equiv) in portions and TEA (1.06 g, 10.47 mmol, 1.00 equiv) dropwise at room temperature. The resulting mixture was stirred for 2 h at room temperature. LCMS indicated the reaction was completely converted to intermediate (MS=368). To the above mixture was added N-{2-[2-(4-amino-2-chlorophenyl)ethoxy]ethyl}-N,4-dimethylbenzenesulfonamide (4.0 g, 10.44 mmol, 1 equiv) and DMAP (3.83 g, 31.34 mmol, 3 equiv) in portions over 10 min at room temperature. The resulting mixture was stirred for additional overnight at 60° C. LCMS indicated the reaction was completed. The reaction mixture was allowed to room temperature. The reaction mixture was purified by reverse flash chromatography with the following conditions: C18 column; mobile phase, ACN in water (0.10% FA), 10% to 60% gradient in 45 min; detector, UV 254/220 nm. The collected fraction was concentrated under vacuum. This resulted in 1-(3-chloro-4-{2-[2-(N-methyl4-methylbenzenesulfonamido)ethoxy]ethyl}phenyl)-3-{[2-(2,6-dioxopiperidin-3-yl)-1-oxo-3H-isoindol-5-yl]methyl}urea (Compound 18-5, 3.2 g, 45%) as a yellow solid. LCMS: (ES, m/z): 682,684 [M+1]⁺.

### Step 4. Synthesis of Compound 18-6

The mixture of 1-(3-chloro-4-{2-[2-(N-methyl4-methylbenzenesulfonamido)ethoxy]ethyl}phenyl)-3-{[2-(2,6-dioxopiperidin-3-yl)-1-oxo-3H-isoindol-5-yl]methyl}urea (Compound 18-5, 4.3 g, 6.30 mmol, 1 equiv) in HBr (30% in AcOH, 86 mL) was stirred for overnight at room temperature. LCMS indicated the reaction was completed. The resulting mixture was concentrated under vacuum. The mixture was basified to pH 7 with saturated NaHCO₃(aq.). The mixture was extracted with DCM (3*50 mL). The combined organic layer was concentrated to dryness under vacuum. The residue was purified by reverse flash chromatography with the following conditions: C18 column; mobile phase, ACN in water (0.1% FA), 10% to 50% gradient in 40 min; detector, UV 254 nm. The collected fraction was concentrated under reduced pressure. This resulted in 1-(3-chloro-4-{2-[2-(methylamino)ethoxy]ethyl}phenyl)-3-{[2-(2,6-dioxopiperidin-3-yl)-1-oxo-3H-isoindol-5-yl]methyl}urea (Compound 18-6, 2.5 g, 75%) as a yellow solid. LCMS: (ES·m/z): 528 [M+1]⁺.

### Step 5. Synthesis of Compound 18-7

To a stirred mixture of 1-(3-chloro-4-{2-[2-(methylamino)ethoxy]ethyl}phenyl)-3-{[2-(2,6-dioxopiperidin-3-yl)-1-oxo-3H-isoindol-5-yl]methyl}urea (Compound 18-6, 700 mg, 1.32 mmol, 1 equiv) in THE (7 mL) was added sat. NaHCO₃ dropwise to pH=8˜9 at 0° C. under nitrogen atmosphere. To the above mixture was added Boc₂O (450 mg, 2.06 mmol, 1.56 equiv) in portions at 0° C. The resulting mixture was stirred for additional 2 h at room temperature. LCMS indicated the reaction was completed. The mixture was extracted with DCM (3*50 mL). The combined organic layer was concentrated to dryness under vacuum. The residue was purified by silica gel column chromatography, eluted with CH₂Cl₂/MeOH (10:1) to afford tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 18-6, 402 mg, 48%) as a white solid. LCMS: (ES·m/z): 528 [M+1-100]⁺, 572 [M+1-56]⁺.

### Step 6. Synthesis of Compound 18-8

To a stirred solution of tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 18-7, 500 mg, 0.79 mmol, 1 equiv) and K₂CO₃ (220.0 mg, 1.59 mmol, 2 equiv) in NMP (5 mL) was stirred for 30 min at room temperature under nitrogen atmosphere. To the above mixture was added prop-2-en-1-yl N-[(chloromethylcarbamoyl)methyl]carbamate (328 mg, 1.59 mmol, 2 equiv) dropwise at 0° C. The resulting mixture was stirred for additional overnight at room temperature. LCMS indicated the reaction was completed. The reaction mixture was purified by reverse flash chromatography with the following conditions: column, C18 silica gel; mobile phase, ACN in water (0.05% TFA), 5% to 80% gradient in 40 min; detector, UV 254 nm. This resulted in crude product (300 mg) as a white solid. The crude product (300 mg) was purified by Prep-HPLC with the following conditions (Column: XBridge Shield RP18 OBD Column, 30*150 mm, 5 m; Mobile Phase A: Water (0.1% FA), Mobile Phase B: ACN; Flow rate: 60 mL/min; Gradient: 30% B to 53% B in 10 min, 53% B; WaveLength: 254 nm; RT1 (min): 9.18; The collected fraction was lyophilized to afford tert-butyl N-(2-{2-[2-chloro-4-({[(2-{2,6-dioxo-1-[(2-{[(prop-2-en-1-yloxy)carbonyl]amino}acetamido)methyl]piperidin-3-yl}-1-oxo-3H-isoindol-5-yl)methyl]carbamoyl}amino)phenyl]ethoxy}ethyl)-N-methylcarbamate (Compound 18-8, 140 mg, 20%) as a white solid. LCMS (ESI, ms):798 [M+H]⁺.

### Step 7. Synthesis of Compound 18-9

To a stirred mixture of tert-butyl N-(2-{2-[2-chloro-4-({[(2-{2,6-dioxo-1-[(2-{[(prop-2-en-1-yloxy)carbonyl]amino}acetamido)methyl]piperidin-3-yl}-1-oxo-3H-isoindol-5-yl)methyl]carbamoyl}amino)phenyl]ethoxy}ethyl)-N-methylcarbamate (Compound 18-8, 500 mg, 0.62 mmol, 1.00 equiv) in THE (6 mL) was added Pd(PPh₃)₄ (72 mg, 0.06 mmol, 0.1 equiv), phenylsilane (135 mg, 1.25 mmol, 2 equiv) at 0 degrees C. under nitrogen atmosphere. The resulting mixture was stirred for 3 h at 25 degrees C. under nitrogen atmosphere. LCMS indicated the reaction was completed. The reaction mixture was purified by reverse flash chromatography with the following conditions: column, C18 silica gel; mobile phase, water (0.05% TFA), ACN (5% to 50% gradient in 30 min); detector, UV 254 nm. This resulted in tert-butyl N-(2-{2-[4-({[(2-{1-[(2-aminoacetamido)methyl]-2,6-dioxopiperidin-3-yl}-1-oxo-3H-isoindol-5-yl)methyl]carbamoyl}amino)-2-chlorophenyl]ethoxy}ethyl)-N-methylcarbamate (Compound 18-7, 400 mg, 89%) as a yellow solid. LCMS (ES, m/z): 714 [M+H]⁺.

### Synthesis of Compound 18-10

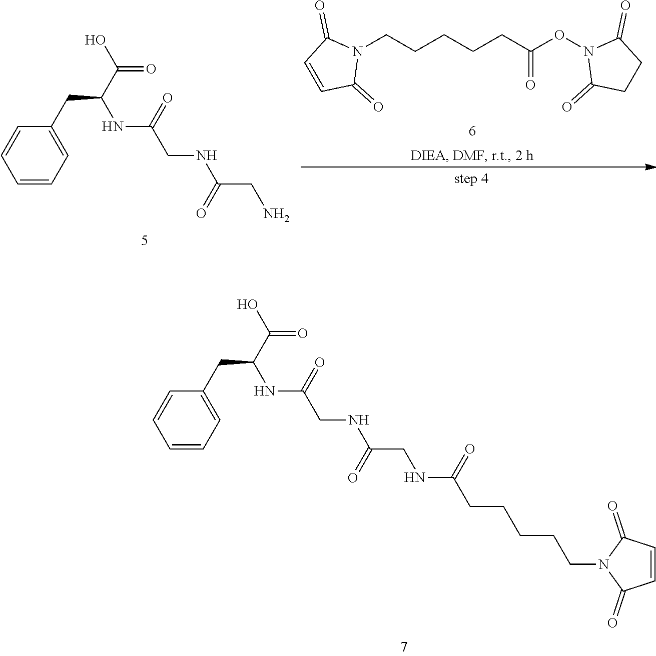 [SMILES: NCC(=O)NCC(=O)N[C@@H](Cc1ccccc1)C(=O)O ; O=C(CCCCCN1C(=O)C=CC1=O)ON1C(=O)CCC1=O ; O=C(CCCCCN1C(=O)C=CC1=O)NCC(=O)NCC(=O)N[C@@H](Cc1ccccc1)C(=O)O]

### Step 4

To a stirred mixture of (2S)-2-[2-(2-aminoacetamido)acetamido]-3-phenylpropanoic acid (1.00 g, 3.58 mmol, 1.00 equiv) and DIEA (0.66 g, 5.10 mmol, 1.50 equiv) in DMF (18 mL) was added 2,5-dioxopyrrolidin-1-yl 6-(2,5-dioxopyrrol-1-yl)hexanoate (1.10 g, 3.57 mmol, 1.11 equiv) at 0° C. The resulting mixture was stirred for 2 h at 25° C. LCMS indicated the reaction was completed. The reaction mixture was purified by reverse flash chromatography with the following conditions: column, C18 silica gel (330 g, 20-40 um); mobile phase, water (containing 0.1% FA), ACN (5% to 50% gradient in 30 min); detector, UV 254 nm. This resulted in (2S)-2-(2-[2-[6-(2,5-dioxopyrrol-1-yl)hexanamido]acetamido]acetamido)-3-phenylpropanoic acid (Compound 18-10, 500 mg, 29%) as a white solid. LCMS (ES, m/z): 473 [M+H]⁺

### Step 8. Synthesis of Compound 18-11

To a stirred solution of (2S)-2-(2-{2-[6-(2,5-dioxopyrrol-1-yl)hexanamido]acetamido}acetamido)-3-phenylpropanoic acid (Compound 18-10, 158 mg, 0.33 mmol, 1.20 equiv) and HATU (127 mg, 0.33 mmol, 1.2 equiv) in DMF (4 mL) was added HOBT (37 mg, 0.28 mmol, 1 equiv) at 0° C. under nitrogen atmosphere. The resulting mixture was stirred for 30 min at 0° C. under nitrogen atmosphere. To the above mixture was added tert-butyl N-(2-{2-[4-({[(2-{1-[(2-aminoacetamido)methyl]-2,6-dioxopiperidin-3-yl}-1-oxo-3H-isoindol-5-yl)methyl]carbamoyl}amino)-2-chlorophenyl]ethoxy}ethyl)-N-methylcarbamate (Compound 18-9, 200 mg, 0.28 mmol, 1 equiv) and DIEA (72 mg, 0.56 mmol, 2 equiv) at 0° C. The resulting mixture was stirred for additional 2 h at ° C. LCMS indicated the reaction was completed. The reaction mixture was purified by reverse flash chromatography with the following conditions: column, C18 silica gel; mobile phase, ACN in water, 5% to 80% gradient in 40 min; detector, UV 254 nm. The collected fraction was lyophilized to afford tert-butyl N-{2-[2-(2-chloro-4-{[({2-[1-({2-[(2S)-2-(2-{2-[6-(2,5-dioxopyrrol-1-yl)hexanamido]acetamido}acetamido)-3-phenylpropanamido]acetamido}methyl)-2,6-dioxopiperidin-3-yl]-1-oxo-3H-isoindol-5-yl}methyl)carbamoyl]amino}phenyl)ethoxy]ethyl}-N-methylcarbamate (Compound 18-8, 175 mg, 53%) as a yellow solid. LCMS (ESI, ms):1168 [M+H]⁺.

### Step 9. Synthesis of Compound (XVIII)

To a stirred solution of tert-butyl N-{2-[2-(2-chloro-4-{[({2-[1-({2-[(2S)-2-(2-{2-[6-(2,5-dioxopyrrol-1-yl)hexanamido]acetamido}acetamido)-3-phenylpropanamido]acetamido}methyl)-2,6-dioxopiperidin-3-yl]-1-oxo-3H-isoindol-5-yl}methyl)carbamoyl]amino}phenyl)ethoxy]ethyl}-N-methylcarbamate (Compound 18-11, 70 mg, 0.06 mmol, 1 equiv) in DCM (700 uL) was added TFA (140 uL) dropwise at 0° C. under nitrogen atmosphere. The resulting mixture was stirred for 1 h at 0° C. under nitrogen atmosphere. LCMS indicated complete reaction. The resulting mixture was concentrated under reduced pressure. The crude product (50 mg) was purified by Prep-HPLC with the following conditions (Column: XSelect CSH Prep C18 OBD Column, 19*250 mm, 5 m; Mobile Phase A: Water (0.1% FA), Mobile Phase B: ACN; Flow rate: 25 mL/min; Gradient: 19% B to 49% B in 7 min, 49% B; Wave Length: 254 nm; RT1 (min): 5; The collected fraction was lyophilized to afford N-{[({[(1S)-1-({[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)carbamoyl]methyl}carbamoyl)-2-phenylethyl]carbamoyl}methyl)carbamoyl]methyl}-6-(2,5-dioxopyrrol-1-yl)hexanamide (Compound (XVIII), 11.5 mg, 17%) as a white solid. LCMS (ESI, ms):1068 [M+H−FA]^{+ 1}H NMR (300 MHz, DMSO-d₆) δ 8.90 (s, 1H), 8.40 (br s, 1H), 8.22-8.18 (m, 1H), 8.14-8.06 (m, 3H), 7.99-7.95 (m, 1H), 7-72-7.66 (m, 2H), 7.49-7.42 (m, 2H), 7.27-7.16 (m, 7H), 6-99 (s, 2H), 6.91 (t, J=5.7 Hz, 1H), 5.21-5.14 (m, 2H), 4.98-4.94 (m, 1H), 4.48-4.39 (m, 5H), 3.77-3.53 (m, 10H), 3.09-3.01 (m, 5H), 2.89 (t, J=7.2 Hz, 2H), 2.78-2.73 (m, 2H), 2.55-2.50 (m, 3H), 2.37-2.26 (m, 2H), 2.07-2.03 (m, 3H), 1.50-1.43 (m, 4H), 1.20-1.15 (m, 2H).

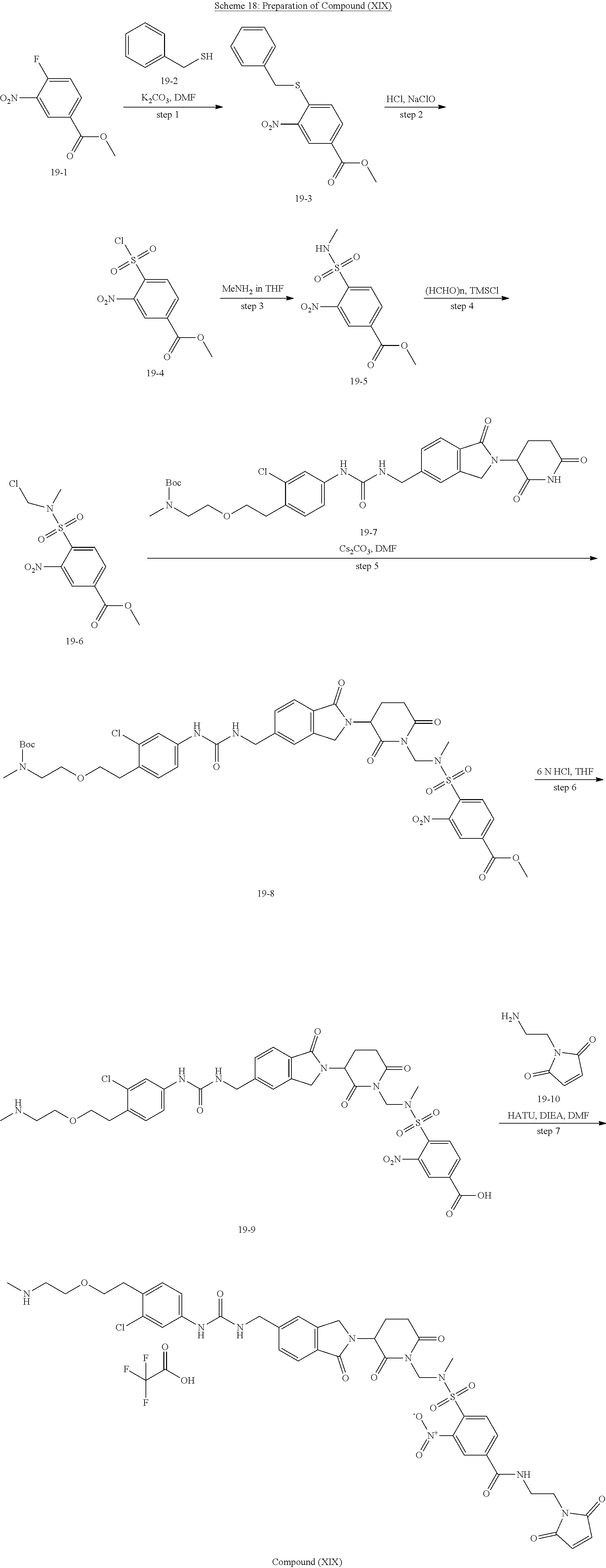

### Step 1. Synthesis of Compound 19-3

To a stirred mixture of methyl 4-fluoro-3-nitrobenzoate (10 g, 50.21 mmol, 1 equiv) and K₂CO₃ (13.88 g, 100.43 mmol, 2 equiv) in DMF (160 mL) was added benzyl mercaptan (12.47 g, 100.43 mmol, 2 equiv) dropwise at 0° C. The resulting mixture was stirred for 3 h at 25° C. TLC indicated the reaction was completed. The reaction was quenched by the addition of water (450 mL) at 0° C. The precipitated solids were collected by filtration and washed with water (3×150 mL). The solid was purified by trituration with PE (300 mL). This resulted in methyl 4-(benzylsulfanyl)-3-nitrobenzoate (Compound 19-3, 10 g, 65%) as a yellow solid. LCMS (ES, m/z): 304 [M+H]⁺

### Step 2. Synthesis of Compound 19-4

To a stirred mixture of [4-(benzylsulfanyl)-3-nitrophenyl]methanol (Compound 19-3, 10 g, 36.32 mmol, 1 equiv) in DCM (200 mL) was added HCl (200 mL, 4N) and NaClO (100 mL, 30%) dropwise at 0° C. The resulting mixture was stirred for 1 h at 0° C. LCMS indicated the reaction was completed. The resulting mixture was extracted with CH₂Cl₂ (3×20 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (1:4) to afford methyl 4-(chlorosulfonyl)-3-nitrobenzoate (Compound 19-4, 8 g, 78%) as a yellow solid. LCMS (ES, m/z): 278 [M−H]⁻

### Step 3. Synthesis of Compound 19-5

To a stirred mixture of methyl 4-(chlorosulfonyl)-3-nitrobenzoate (Compound 19-4, 5 g, 17.87 mmol, 1 equiv) in THE (60 mL) was added CH₃NH₂ (60 mL, 1N in THF) dropwise at 0° C. The resulting mixture was stirred for 3 h at 25° C. LCMS indicated the reaction was completed. The resulting mixture was concentrated under reduced pressure. The residue was purified by silica gel column chromatography, eluted with PE/EA (1:1) to afford methyl 4-(methylsulfamoyl)-3-nitrobenzoate (Compound 19-5, 2.5 g, 50%) as a yellow solid. LCMS (ES, m/z): 273 [M−H]⁻

### Step 4. Synthesis of Compound 19-6

To a stirred mixture of methyl 4-(methylsulfamoyl)-3-nitrobenzoate (Compound 19-5, 600 mg, 2.18 mmol, 1 equiv) in DCM (15 mL) was added TMSCl (475 mg, 4.37 mmol, 2 equiv) dropwise at 0° C. The resulting mixture was stirred for 3 h at 25° C. LCMS indicated the reaction was completed. The resulting mixture was concentrated under reduced pressure. This resulted in methyl 4-[chloromethyl(methyl)sulfamoyl]-3-nitrobenzoate (Compound 19-6, 600 mg, 84%) as a white solid. The crude product was used to next step without further purification. LCMS (ES, m/z): 341 [M+Na]⁺, (MeOH derivative).

### Step 5. Synthesis of Compound 19-8

To a stirred mixture of tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 19-7, prepared as described for Compound 11-2, 583 mg, 0.92 mmol, 1 equiv) and Cs₂CO₃ (302 mg, 0.92 mmol, 1 equiv) in DMF (15 mL) was added methyl 4-[chloromethyl(methyl)sulfamoyl]-3-nitrobenzoate (600 mg, 1.85 mmol, 2 equiv) at 0° C. The resulting mixture was stirred for 0.5 h at 0° C. LCMS indicated the reaction was completed. The reaction mixture was purified by reverse flash chromatography with the following conditions: column, C18 silica gel; mobile phase, water (0.05% TFA), ACN 5% to 100% gradient in 30 min; detector, UV 254 nm. This resulted in methyl 4-{[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)sulfamoyl}-3-nitrobenzoate (Compound 19-8, 250 mg, 26%) as a white solid. LCMS (ES, m/z): 814 [M+H−Boc]⁺

### Step 6. Synthesis of Compound 19-9

To a stirred mixture of methyl 4-{[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)sulfamoyl}-3-nitrobenzoate (Compound 19-8, 200 mg, 0.21 mmol, 1 equiv) in THE (0.8 mL) was added HCl (6 N, 0.8 mL) at room temperature. The resulting mixture was stirred for 16 h at room temperature. LCMS indicated the reaction was completed. The mixture was purified by reverse flash chromatography with the following conditions: column, C18 silica gel; mobile phase, water (0.05% TFA), ACN 5% to 50% gradient in 30 min; detector, UV 254 nm. This resulted in 4-[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)(methyl)sulfamoyl]-3-nitrobenzoic acid (Compound 19-9, 60 mg, 30%) as a white solid. LCMS (ES, m/z): 800 [M+H]⁺

### Step 7. Synthesis of Compound (XIX)

To a stirred mixture of 4-[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)(methyl)sulfamoyl]-3-nitrobenzoic acid (55 mg, 0.069 mmol, 1 equiv), 1-(2-aminoethyl)pyrrole-2,5-dione (10 mg, 0.076 mmol, 1.1 equiv) and DIEA (26 mg, 0.20 mmol, 3 equiv) in DMF (0.5 mL) was added HATU (31 mg, 0.083 mmol, 1.2 equiv) at 0° C. The resulting mixture was stirred for 4 h at 25° C. LCMS indicated the reaction was completed. The residue was purified by reverse flash chromatography with the following conditions: column, C18 silica gel; mobile phase, water (0.05% TFA), ACN 5% to 50% gradient in 30 min; detector, UV 254 nm. The crude product was purified by Prep-HPLC with the following conditions Column: Xselect CSH F-Phenyl OBD column, 19×250 mm, 5 m; Mobile Phase A: Water (0.05% TFA), Mobile Phase B: ACN; Flow rate: 25 mL/min; Gradient: 21% B to 41% B in 8 min, 41% B; Wave Length: 254 nm; RT1 (min): 7.27; The collected fraction was lyophilized to afford 4-[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)(methyl)sulfamoyl]-N-[2-(2,5-dioxopyrrol-1-yl)ethyl]-3-nitrobenzamide; trifluoroacetic acid (Compound (XIX), 4.3 mg, 5%) as a white solid. LCMS (ES, m/z): 922 [M+H]⁺. ¹H-NMR (CD₃OD, 400 MHz) (ppm): 8.27-7.88 (m, 3H), 7.82-7.46 (m, 4H), 7.23 (d, J=1.2 Hz, 2H), 6.79 (s, 2H), 5.47-5.25 (m, 2H), 5.15-5.10 (m, 1H), 4.63-4.28 (m, 4H), 3.80-3.70 (m, 6H), 3.65-3.54 (m, 2H), 3.25-3.15 (m, 2H), 3.09 (d, J=2.4 Hz, 3H), 3.04-2.96 (m, 2H), 2.96-2.86 (m, 2H), 2.71 (s, 3H), 2.48-2.26 (m, 1H), 2.24-2.03 (m, 1H).

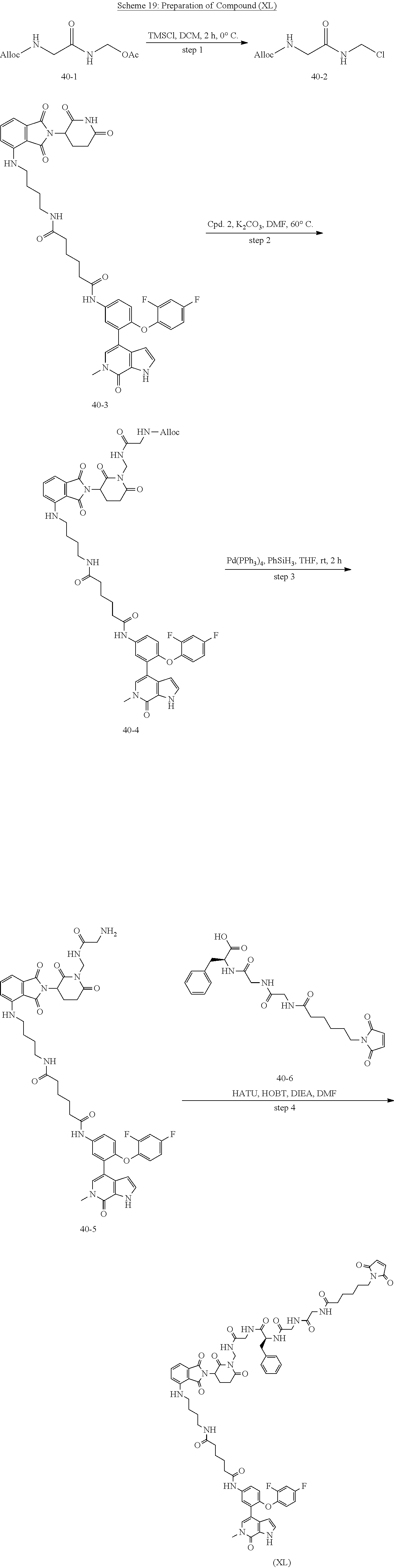

### Step 1. Synthesis of Compound 40-2

To a stirred solution of (2-{[(prop-2-en-1-yloxy)carbonyl]amino}acetamido)methyl acetate (Compound 40-1, 400 mg, 1.73 mmol, 1 equiv) in DCM (8 mL) were added TMSCl (755 mg, 6.94 mmol, 4 equiv) in portions at 0° C. under nitrogen atmosphere. The resulting mixture was stirred for 2 h at room temperature under nitrogen atmosphere. LCMS indicated the reaction was completed. The resulting mixture was concentrated under reduced pressure. The crude product mixture was used in the next step directly without further purification. LCMS (ES, m/z): 203 [M+H]⁺ (derivated with methanol).

### Step 2. Synthesis of Compound 40-4

To a stirred mixture of N′-[4-(2,4-difluorophenoxy)-3-{6-methyl-7-oxo-1H-pyrrolo[2,3-c]pyridin-4-yl}phenyl]-N-(4-{[2-(2,6-dioxopiperidin-3-yl)-1,3-dioxoisoindol-4-yl]amino}butyl)hexanediamide (Compound 40-3, prepared according to the procedure described in https://doi.org/10.1016/j.bioorg.2021.105238, 300 mg, 0.36 mmol, 1 equiv) and K₂CO₃ (151 mg, 1.09 mmol, 3 equiv) in DMF (7 mL) were added prop-2-en-1-yl N-[(methoxymethylcarbamoyl)methyl]carbamate (147 mg, 0.73 mmol, 2 equiv) dropwise at 0° C. under nitrogen atmosphere. The resulting mixture was stirred for 2 h at 60° C. under nitrogen atmosphere. LCMS indicated the reaction was completed. The reaction mixture was purified by reverse flash chromatography with the following conditions: column, C18 silica gel; mobile phase, ACN in Water (0.05% TFA), 10% to 50% gradient in 30 min; detector, UV 254 nm. This resulted in prop-2-en-1-yl N-[({[3-(4-{[4-(5-{[4-(2,4-difluorophenoxy)-3-{6-methyl-7-oxo-1H-pyrrolo[2,3-c]pyridin-4-yl}phenyl]carbamoyl}pentanamido)butyl]amino}-1,3-dioxoisoindol-2-yl)-2,6-dioxopiperidin-1-yl]methyl}carbamoyl)methyl]carbamate (Compound 40-4, 200 mg, 56%) as a yellow solid. LCMS (ES, m/z): 992 [M+H]⁺

### Step 3. Synthesis of Compound 40-5

To a stirred mixture of prop-2-en-1-yl N-[({[3-(4-{[4-(5-{[4-(2,4-difluorophenoxy)-3-{6-methyl-7-oxo-1H-pyrrolo[2,3-c]pyridin-4-yl}phenyl]carbamoyl}pentanamido)butyl]amino}-1,3-dioxoisoindol-2-yl)-2,6-dioxopiperidin-1-yl]methyl}carbamoyl)methyl]carbamate (Compound 40-4, 100 mg, 0.10 mmol, 1 equiv) and Pd(PPh₃)₄ (11 mg, 0.01 mmol, 0.1 equiv) in THE (1 mL) was added phenylsilane (21 mg, 0.20 mmol, 2 equiv) in portions at room temperature under nitrogen atmosphere. The resulting mixture was stirred for 2 h at room temperature under nitrogen atmosphere. LCMS indicated the reaction was completed. The resulting mixture was concentrated under reduced pressure. The residue was purified by reverse flash chromatography with the following conditions: column, C18 silica gel; mobile phase, ACN in Water (0.05% TFA), 10% to 50% gradient in 30 min; detector, UV 254 nm. This resulted in N-{4-[(2-{1-[(2-aminoacetamido)methyl]-2,6-dioxopiperidin-3-yl}-1,3-dioxoisoindol-4-yl)amino]butyl}-N′-[4-(2,4-difluorophenoxy)-3-{6-methyl-7-oxo-1H-pyrrolo[2,3-c]pyridin-4-yl}phenyl]hexanediamide (80 mg, 87%) as a yellow solid. LCMS (ES, m/z): 930 [M+Na]⁺

### Step 4. Synthesis of Compound (XL)

To a stirred solution of (2S)-2-(2-{2-[6-(2,5-dioxopyrrol-1-yl)hexanamido]acetamido}acetamido)-3-phenylpropanoic acid (Compound 40-6, prepared as described in Compound 18-10, 18 mg, 0.040 mmol, 1.2 equiv) in DMF (0.4 mL) was added HATU (15 mg, 0.04 mmol, 1.2 equiv) dropwise at 0° C. under nitrogen atmosphere. To the above mixture was added N-{4-[(2-{1-[(2-aminoacetamido)methyl]-2,6-dioxopiperidin-3-yl}-1,3-dioxoisoindol-4-yl)amino]butyl}-N′-[4-(2,4-difluorophenoxy)-3-{6-methyl-7-oxo-1H-pyrrolo[2,3-c]pyridin-4-yl}phenyl]hexanediamide (Compound 40-5, 30 mg, 0.033 mmol, 1 equiv) and DIEA (13 mg, 0.10 mmol, 3 equiv) dropwise at room temperature. The resulting mixture was stirred for additional 2 h at room temperature. LCMS indicated the reaction was completed. The residue was purified by reverse flash chromatography with the following conditions: column, C18 silica gel; mobile phase, ACN in Water (0.05% TFA), 10% to 50% gradient in 30 min; detector, UV 254 nm. This resulted in N′-[4-(2,4-difluorophenoxy)-3-{6-methyl-7-oxo-1H-pyrrolo[2,3-c]pyridin-4-yl}phenyl]-N-[4-({2-[1-({2-[(2S)-2-(2-{2-[6-(2,5-dioxopyrrol-1-yl)hexanamido]acetamido}acetamido)-3-phenylpropanamido]acetamido}methyl)-2,6-dioxopiperidin-3-yl]-1,3-dioxoisoindol-4-yl}amino)butyl]hexanediamide; trifluoroacetic acid (3.5 mg, 7%) as a yellow solid. LCMS (ES, m/z): 682 [M/2]⁺, 1362 [M+H]⁺; ¹H NMR (400 MHz, DMSO-d₆) δ 12.04 (s, 1H), 9.98 (s, 1H), 8.24-8.11 (m, 2H), 8.09-7.99 (m, 2H), 7.97-7.83 (m, 1H), 7.83-7.79 (m, 2H), 7.58-7.52 (m, 2H), 7.35-7.30 (m, 1H), 7.29-7.28 (m, 2H), 7.25-7.19 (m, 4H), 7.18-7.18 (m, 1H), 7.17-7.15 (m, 1H), 7.10-7.03 (m, 4H), 6.99-6.90 (m, 1H), 6.55 (s, 1H), 6.26 (s, 1H), 5.13-5.03 (m, 3H), 4.49-4.45 (m, 1H), 3.76-3.65 (m, 5H), 3.29-3.20 (m, 5H), 3.07-2.97 (m, 5H), 2.80-2.74 (m, 2H), 2.33-2.27 (m, 3H), 2.12-2.06 (m, 6H), 1.53-1.44 (m, 12H), 1.23-1.16 (m, 3H)

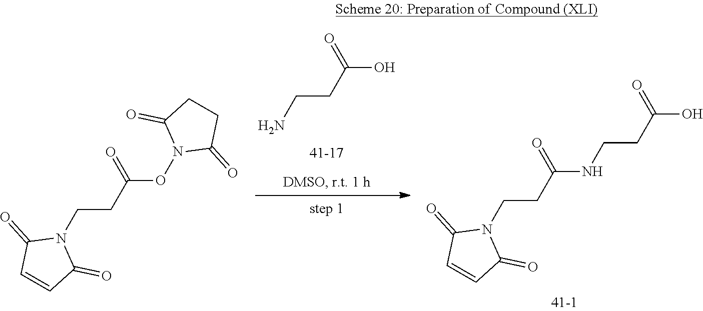 [SMILES: O=C(CCN1C(=O)C=CC1=O)ON1C(=O)CCC1=O ; NCCC(=O)O ; O=C(O)CCNC(=O)CCN1C(=O)C=CC1=O]

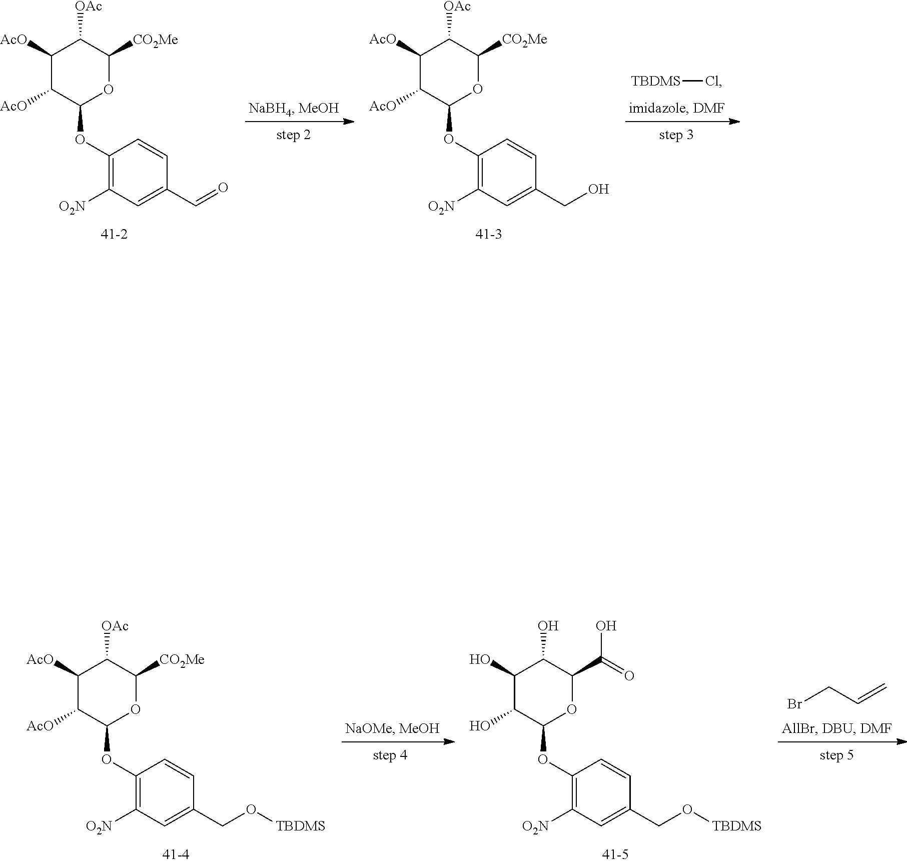

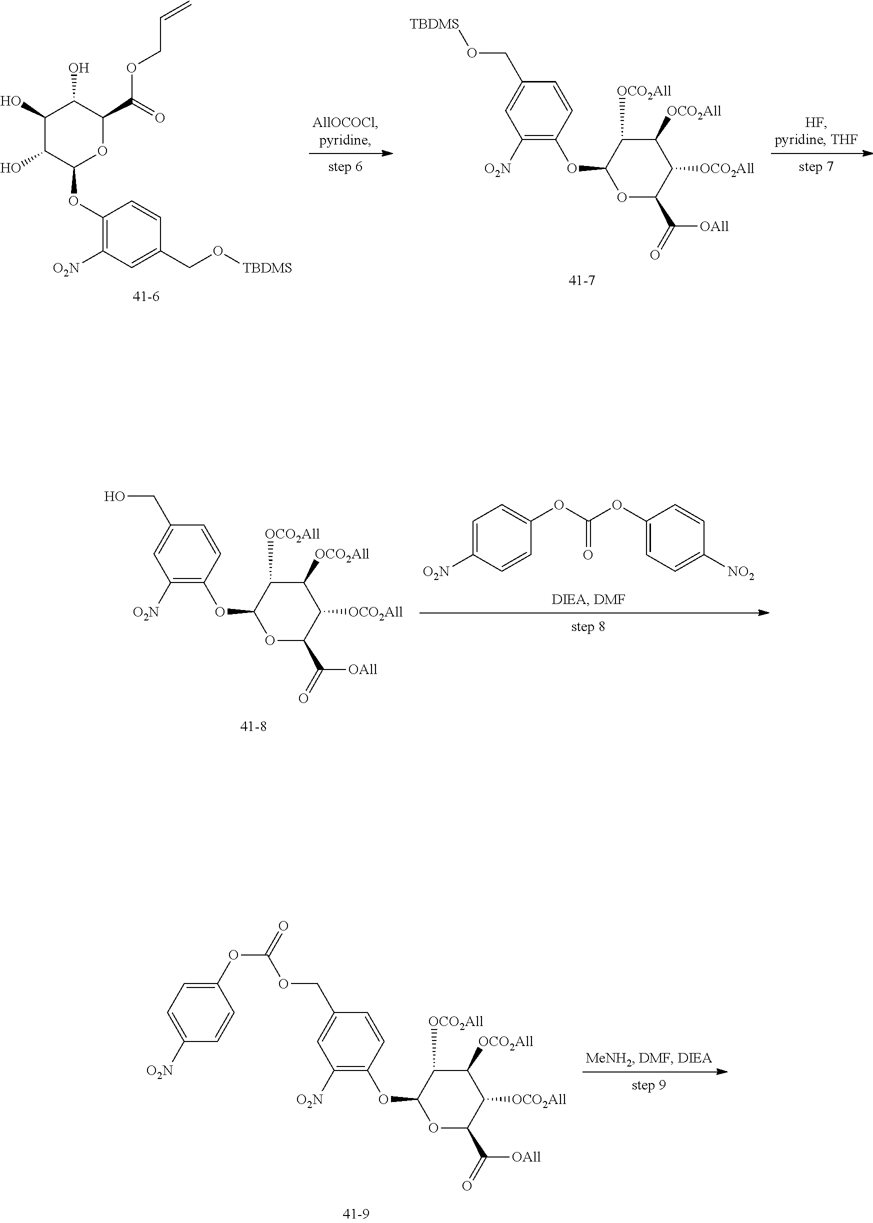

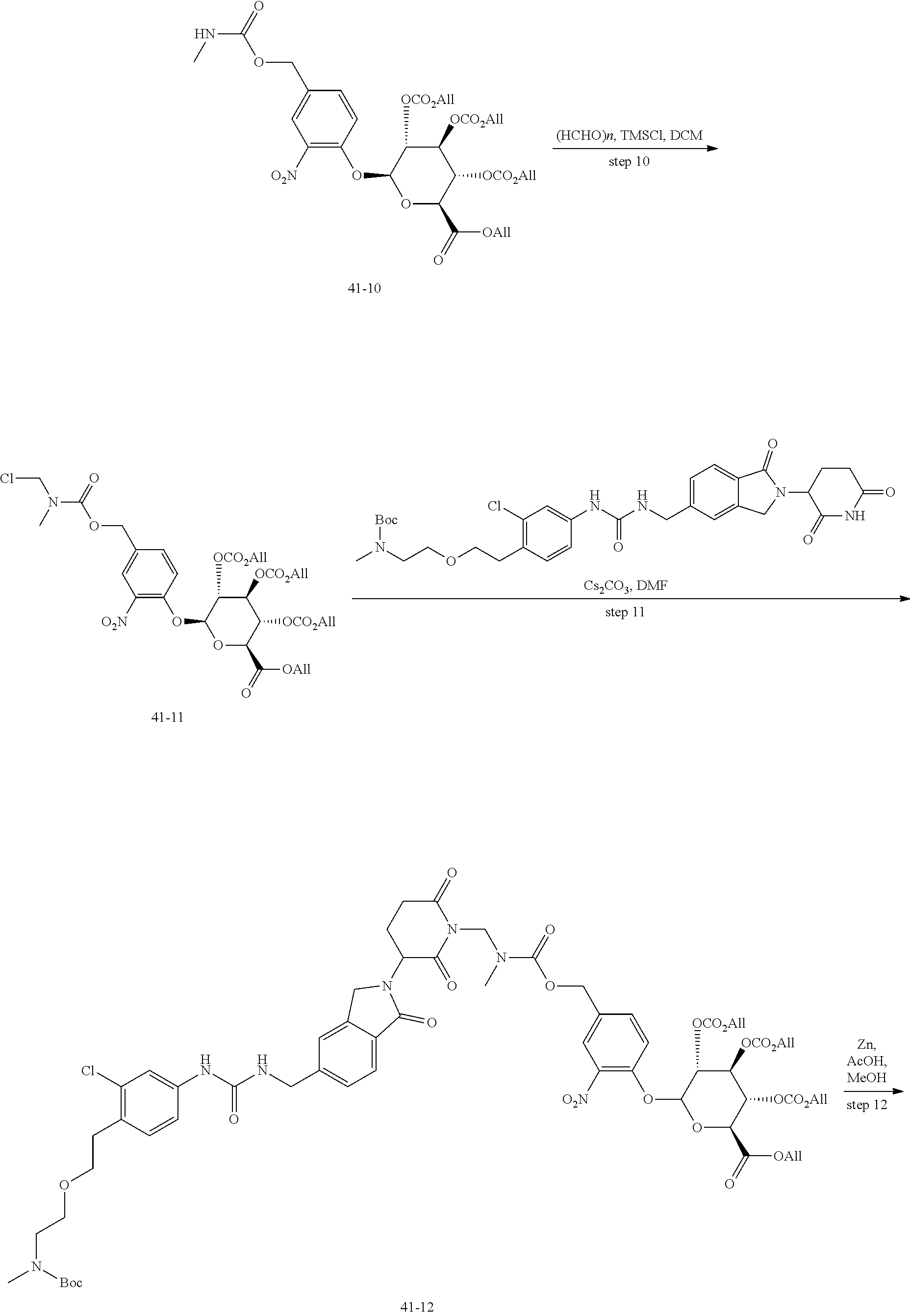

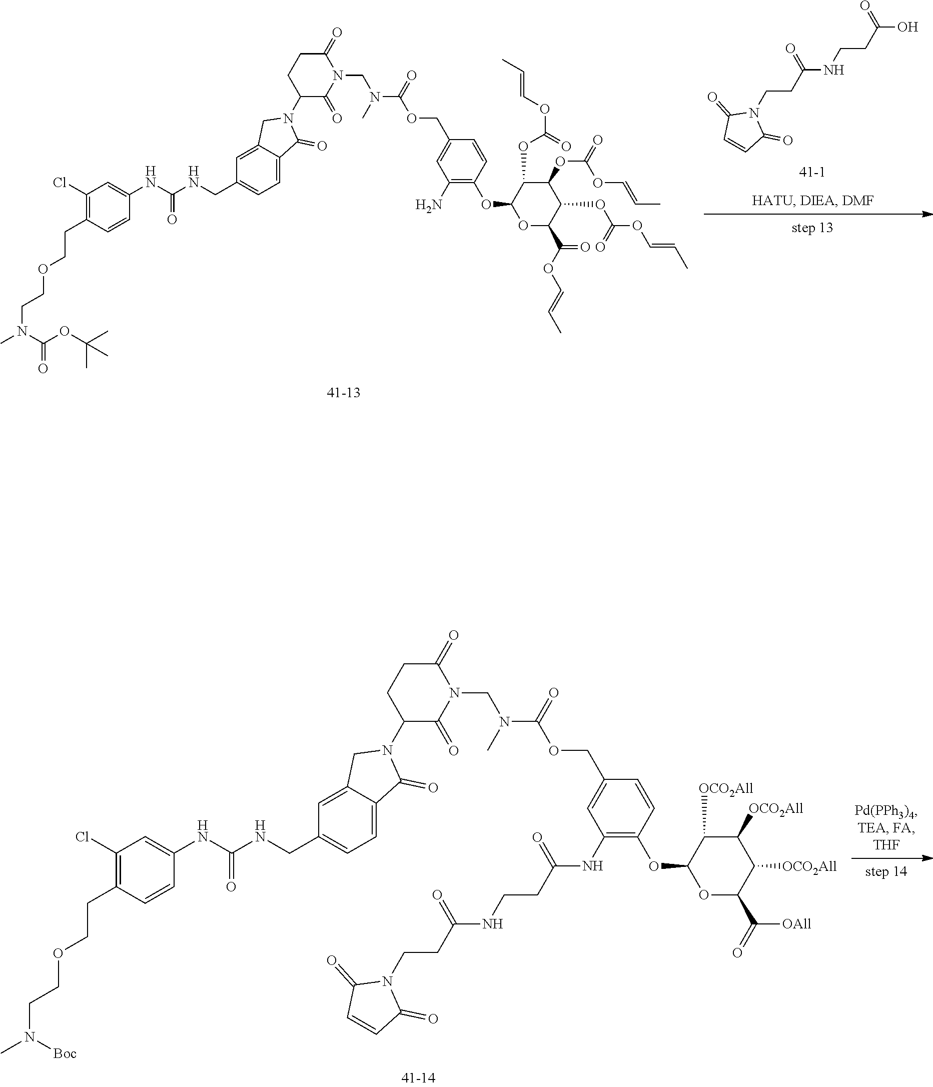

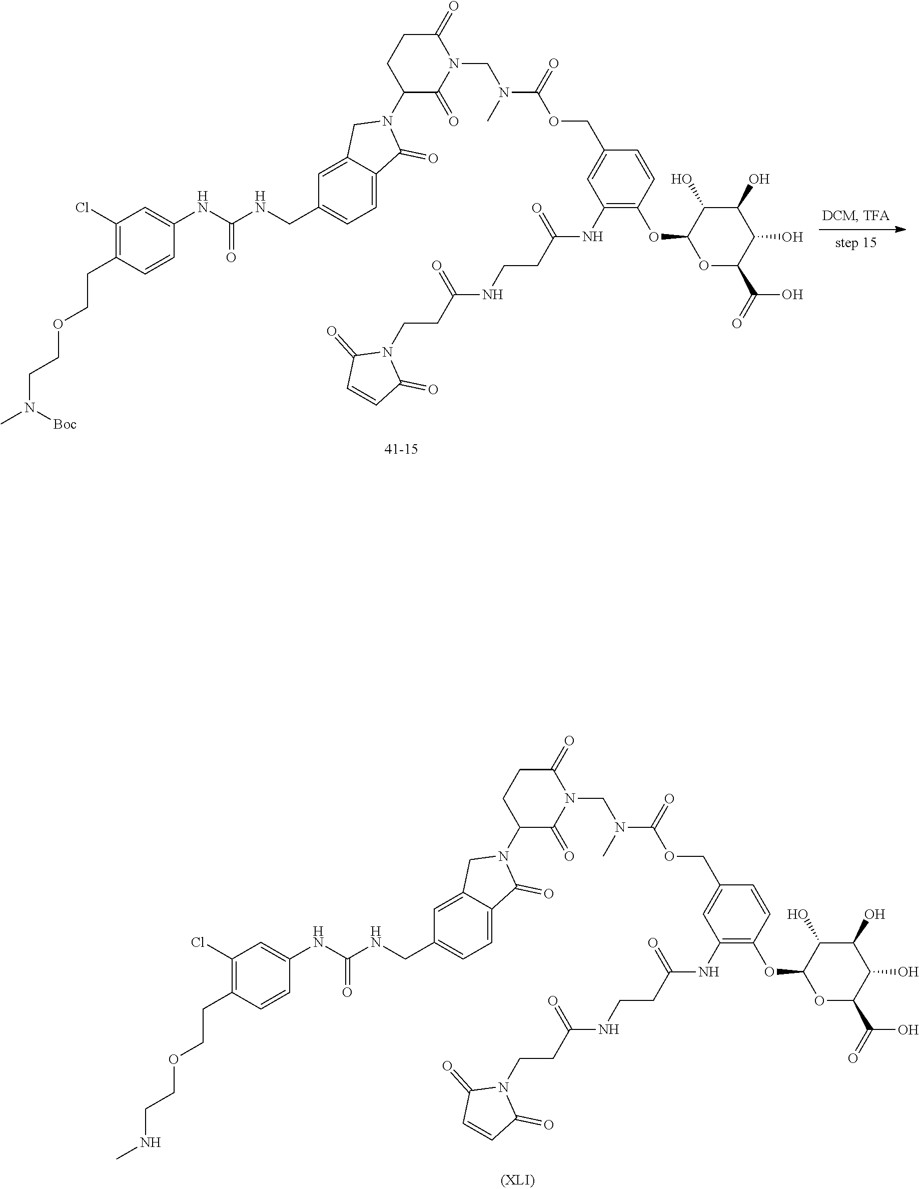 [SMILES: CN(CN1C(=O)CCC(N2Cc3cc(CNC(=O)Nc4ccc(CCOCCN(C)C(=O)OC(C)(C)C)c(Cl)c4)ccc3C2=O)C1=O)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1 ; CNCCOCCc1ccc(NC(=O)NCc2ccc3c(c2)CN(C2CCC(=O)N(CN(C)C(=O)OCc4ccc(O[C@@H]5O[C@H](C(=O)O)[C@@H](O)[C@H](O)[C@H]5O)c(NC(=O)CCNC(=O)CCN5C(=O)C=CC5=O)c4)C2=O)C3=O)cc1Cl]

### Step 1. Synthesis of Compound 41-1

To a stirred solution of 2,5-dioxopyrrolidin-1-yl 3-(2,5-dioxopyrrol-1-yl) propanoate (5.0 g, 18.78 mmol, 1.0 equiv) in DMSO (50 mL) were added β-alanine (Compound 41-17, 2.01 g, 22.53 mmol, 1.2 equiv) in portions at room temperature under nitrogen atmosphere. The resulting mixture was stirred for 6 h at room temperature under nitrogen atmosphere. LCMS indicated the reaction was completed. The reaction mixture was purified by reverse flash chromatography with the following conditions: column, C18 silica gel; mobile phase, ACN in water (0.1% FA), 0% to 10% gradient in 30 min; detector, UV 220 nm. The collected fraction was lyophilized to afford 3-[3-(2,5-dioxopyrrol-1-yl) propanamido]propanoic acid (Compound 41-1, 3.0 g, 61%) as a white solid. LCMS: (ES, m/s): 241 [M+H]⁺, 263 [M+Na]⁺.

### Synthesis of Compound 41-2

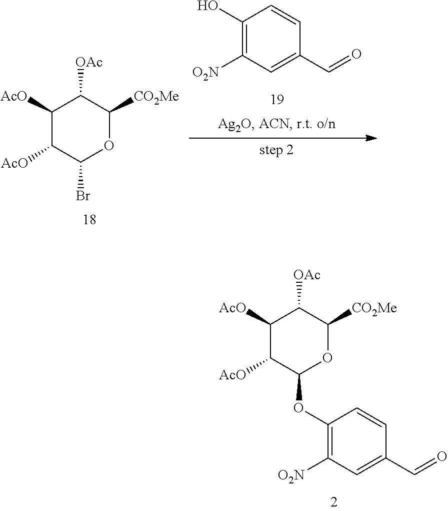 [SMILES: COC(=O)[C@H]1O[C@H](Br)[C@H](OC(C)=O)[C@@H](OC(C)=O)[C@@H]1OC(C)=O ; O=Cc1ccc(O)c([N+](=O)[O-])c1 ; COC(=O)[C@H]1O[C@@H](Oc2ccc(C=O)cc2[N+](=O)[O-])[C@H](OC(C)=O)[C@@H](OC(C)=O)[C@@H]1OC(C)=O]

To a stirred solution of 4-formyl-2-nitrophenol (4.21 g, 25.19 mmol, 1.00 equiv) and Ag2O (7.00 g, 30.20 mmol, 1.20 equiv) in ACN (100 mL, 190.24 mmol, 75.00 equiv) were added methyl (2S,3S,4S,5R,6R)-3,4,5-tris(acetyloxy)-6-bromooxane-2-carboxylate (10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 ml×3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 41-2, 10.5 g, 86%) as a white solid. H-NMR analysis indicated it was the desired product. LCMS (ES, m/z): 484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H),

### Step 2. Synthesis of Compound 41-3

To a stirred solution of methyl (2S,3 S,4S,5R,6S)-3,4,5-tris(acetyloxy)-6-(4-formyl-2-nitrophenoxy)oxane-2-carboxylate (Compound 41-2, 54.6 g, 112.95 mmol, 1.00 equiv) in MeOH (800 mL) were added NaBH₄ (3.4 g, 90.36 mmol, 0.80 equiv) in portions at room temperature under N₂ atmosphere. The resulting mixture was stirred for 2 h at room temperature under N₂ atmosphere. LCMS indicated the reaction was completed. The reaction was quenched with HCl (0.02 mol/L) at room temperature. The resulting mixture was extracted with CH₂Cl₂ (3×300 mL). The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate (Compound 41-3, 44 g, 64%) as a green solid. LCMS (ES, m/z): 486 [M+H]⁺, 508 [M+Na]⁺.

### Step 3. Synthesis of Compound 41-4

To a stirred solution of methyl (2S,3S,4S,5R,6S)-3,4,5-tris(acetyloxy)-6-[4-(hydroxymethyl)-2-nitrophenoxy]oxane-2-carboxylate (Compound 41-3, 28 g, 57.68 mmol, 1.00 equiv) in DMF (300 mL) were added imidazole (5.89 g, 86.52 mmol, 1.50 equiv) and TBDMS-C₁ (13.04 g, 86.52 mmol, 1.50 equiv) in portions at room temperature under N₂ atmosphere. The resulting mixture was stirred for 4 h at room temperature under N₂ atmosphere. LCMS indicated the reaction was completed. The reaction was quenched with water at room temperature. The resulting mixture was extracted with CH₂Cl₂ (3×300 mL). The combined organic was concentrated under vacuum. The residue was purified by silica gel column chromatography, eluted with PE/EA (1:1) to afford methyl (2S,3S,4S,5R,6S)-3,4,5-tris(acetyloxy)-6-(4-{[(tert-butyldimethylsilyl)oxy]methyl}-2-nitrophenoxy)oxane-2-carboxylate (30 g, 80%) as a white solid. LCMS (ES, m/z): 600 [M+H]⁺, 622 [M+Na]⁺; ¹H-NMR (300 MHz, DMSO-d₆): 7.80 (d, J=9 Hz, 1H), 7.65-7.61 (m, 1H), 7.42 (d, J=9 Hz, 1H), 5.72 (d, J=9 Hz, 1H), 5.47 (t, J=9 Hz, 1H), 5.15-5.05 (m, 2H), 4.74 (d, J=4.5 Hz, 3H), 3.65 (s, 3H), 2.02 (t, J=9 Hz, 9H), 0.91 (t, J=9 Hz, 9H), 0.09 (s, 6H).

### Step 4. Synthesis of Compound 41-5

To a stirred solution of methyl (2S,3S,4S,5R,6S)-3,4,5-tris(acetyloxy)-6-(4-{[(tert-butyldimethylsilyl)oxy]methyl}-2-nitrophenoxy)oxane-2-carboxylate (Compound 41-4, 30 g, 50.02 mmol, 1.00 equiv) in MeOH (600 mL) were added NaOMe (16.19 g, 299.68 mmol, 6.0 equiv) in portions at room temperature under N₂ atmosphere. The resulting mixture was stirred f or overnight at room temperature under N₂ atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 10% gradient in 30 min; detector, UV 220 nm. The collected fraction was concentrated under vacuum to afford (2S,3S,4S,5R,6S)-6-(4-{[(tert-butyldimethylsilyl)oxy]methyl}-2-nitrophenoxy)-3,4,5-trihydroxyoxane-2-carboxylic acid (Compound 41-5, 28 g, 92%) as a yellow solid. LCMS (ES, m/z): 477 [M+H₂O]⁺, 482 [M+Na]⁺.

### Step 5. Synthesis of Compound 41-6

To a stirred solution of (2S,3S,4S,5R,6S)-6-(4-{[(tert-butyldimethylsilyl)oxy]methyl}-2-nitrophenoxy)-3,4,5-trihydroxyoxane-2-carboxylic acid (Compound 41-5, 28 g, 46.30 mmol, 1.00 equiv, 76%) in DMF (300 mL) were added DBU (14.10 g, 92.61 mmol, 2.00 equiv) and allyl bromide (16.8 g, 138.92 mmol, 3.00 equiv) in portions at room temperature under N₂ atmosphere. The resulting mixture was stirred for overnight at 40° C. under N₂ atmosphere. Desired product could be detected by LCMS. The resulting mixture was concentrated under vacuum. The reaction was quenched with Water at room temperature. The resulting mixture was extracted with CH₂Cl₂ (3×200 ml). The combined organic layers were concentrated under reduced pressure. The residue was purified by silica gel column chromatography, eluted with CH₂Cl₂/MeOH (9:1) to afford prop-2-en-1-yl (2S,3S,4S,5R,6S)-6-(4-{[(tert-butyldimethylsilyl)oxy]methyl}-2-nitrophenoxy)-3,4,5-trihydroxyoxane-2-carboxylate (19 g, 41%) as a light red oil. LCMS (ES, m/z): 517 [M+H₂O]⁺, 522 [M+Na]⁺.

### Step 6. Synthesis of Compound 41-7

To a stirred solution of prop-2-en-1-yl (2S,3S,4S,5R,6S)-6-(4-{[(tert-butyldimethylsilyl)oxy]methyl}-2-nitrophenoxy)-3,4,5-trihydroxyoxane-2-carboxylate (Compound 41-6, 19 g, 38.03 mmol, 1.00 equiv) in pyridine (300 mL) were added allyl chlorocarbonate (137.47 g, 1140.55 mmol, 29.99 equiv) in portions at room temperature under nitrogen atmosphere. The resulting mixture was stirred for 4 h at room temperature under nitrogen atmosphere. ˜60% desired product could be detected by LCMS. The reaction was quenched with Water at room temperature. The resulting mixture was extracted with CH₂Cl₂ (3×100 mL). The combined organic layers were washed with was concentrated under reduced pressure. The residue was purified by silica gel column chromatography, eluted with PE/EA (3:1) to afford prop-2-en-1-yl (2S,3S,4S,5R,6S)-6-(4-{[(tert-butyldimethylsilyl)oxy]methyl}-2-nitrophenoxy)-3,4,5-tris({[(prop-2-en-1-yloxy)carbonyl]oxy})oxane-2-carboxylate (Compound 41-7, 13.9 g, 43%) as a light red oil. LCMS: (ES, m/s): 769 [M+H₂O]⁺, 774 [M+Na]⁺.

### Step 7. Synthesis of Compound 41-8

To a stirred solution of prop-2-en-1-yl (2S,3S,4S,5R,6S)-6-(4-{[(tert-butyldimethylsilyl)oxy]methyl}-2-nitrophenoxy)-3,4,5-tris({[(prop-2-en-1-yloxy)carbonyl]oxy})oxane-2-carboxylate (Compound 41-7, 13.9 g, 18.48 mmol, 1.00 equiv) in THE (280 mL) were added HF-Pyridine (65 mL, 65%) in portions at room temperature under nitrogen atmosphere. The resulting mixture was stirred for 2 h at room temperature under nitrogen atmosphere. LCMS indicated the reaction was completed. The reaction was quenched with Water at room temperature. The resulting mixture was extracted with CH₂Cl₂ (3×500 mL). The mixture was concentrated under reduced pressure. The residue was purified by silica gel column chromatography, eluted with PE/EA (2:3) to afford prop-2-en-1-yl (2S,3S,4S,5R,6S)-6-[4-(hydroxymethyl)-2-nitrophenoxy]-3,4,5-tris({[(prop-2-en-1-yloxy)carbonyl]oxy})oxane-2-carboxylate (Compound 41-8, 10.5 g, 80%) as a light yellow oil. LCMS: (ES, m/s): 655 [M+H₂O]⁺, 660 [M+Na]⁺.

### Step 8. Synthesis of Compound 41-9

To a stirred solution of prop-2-en-1-yl (2S,3S,4S,5R,6S)-6-[4-(hydroxymethyl)-2-nitrophenoxy]-3,4,5-tris({[(prop-2-en-1-yloxy)carbonyl]oxy})oxane-2-carboxylate (10.5 g, 16.46 mmol, 1.00 equiv) in DMF (100 mL) were added bis(4-nitrophenyl) carbonate (7.52 g, 24.71 mmol, 1.50 equiv) and DIEA (6.39 g, 49.44 mmol, 3.00 equiv) in portions at room temperature under nitrogen atmosphere. The resulting mixture was stirred for 6 h at room temperature under nitrogen atmosphere. LCMS indicated the reaction was completed. The reaction was quenched with Water at room temperature. The resulting mixture was extracted with CH₂Cl₂ (3×500 mL). The combined organic layers were concentrated under reduced pressure. The residue was purified by silica gel column chromatography, eluted with PE/EA (3:2) to afford prop-2-en-1-yl (2S,3S,4S,5R,6S)-6-(2-nitro-4-{[(4-nitrophenoxycarbonyl)oxy]methyl}phenoxy)-3,4,5-tris({[(prop-2-en-1-yloxy)carbonyl]oxy})oxane-2-carboxylate (10.6 g, 74%) as a light yellow semi-solid. LCMS: (ES, m/s): 820 [M+H₂O]⁺, 825 [M+Na]⁺.

### Step 9. Synthesis of Compound 41-10

To a stirred solution of prop-2-en-1-yl (2S,3S,4S,5R,6S)-6-(2-nitro-4-{[(4-nitrophenoxycarbonyl)oxy]methyl}phenoxy)-3,4,5-tris({[(prop-2-en-1-yloxy)carbonyl]oxy})oxane-2-carboxylate (Compound 41-9, 1 g, 1.24 mmol, 1.0 equiv) in DMF (1.0 mL) were added DIEA (0.48 g, 3.73 mmol, 3.0 equiv) and Methylamine hydrochloride (0.12 g, 1.86 mmol, 1.5 equiv) in portions at 0° C. under nitrogen atmosphere. The resulting mixture was stirred for 30 min at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 70% gradient in 30 min; detector, UV 220 nm. The resulting mixture was extracted with CH₂Cl₂ (3×200 mL). The combined organic layers was concentrated under reduced pressure to afford prop-2-en-1-yl (2S,3S,4S,5R,6S)-6-(4-{[(methylcarbamoyl)oxy]methyl}-2-nitrophenoxy)-3,4,5-tris({[(prop-2-en-1-yloxy)carbonyl]oxy})oxane-2-carboxylate (Compound 41-10, 900 mg, 95%) as a semi-solid. LCMS: (ES, m/s): 712 [M+H₂O]⁺, 717 [M+Na]⁺.

### Step 10. Synthesis of Compound 41-11

To a stirred solution of prop-2-en-1-yl (2S,3S,4S,5R,6S)-6-(4-{[(methylcarbamoyl)oxy]methyl}-2-nitrophenoxy)-3,4,5-tris({[(prop-2-en-1-yloxy)carbonyl]oxy})oxane-2-carboxylate (Compound 41-10, 500 mg, 0.72 mmol, 1.0 equiv) in DCM (10 mL) were added Paraformaldehyde (129 mg, 1.44 mmol, 2.00 equiv) and TMSCl (234 mg, 2.16 mmol, 3.00 equiv) in portions at room temperature under nitrogen atmosphere. The resulting mixture was stirred for 1 h at room temperature under nitrogen atmosphere. LCMS indicated the reaction was completed (derivative with MeOH). The resulting mixture was concentrated under vacuum to afford prop-2-en-1-yl (2S,3S,4S,5R,6S)-6-[4-({[chloromethyl(methyl)carbamoyl]oxy}methyl)-2-nitrophenoxy]-3,4,5-tris({[(prop-2-en-1-yloxy)carbonyl]oxy})oxane-2-carboxylate (Compound 41-11, 500 mg, 93%) as a crude product. The crude product was used in the next step directly without further purification. LCMS: (ES, m/s): 756 [M+H₂O]⁺, 761 [M+Na]⁺ (derivative with MeOH)

### Step 11. Synthesis of Compound 41-12

To a stirred solution of tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 41-11, 338 mg, 0.53 mmol, 1.0 equiv) and Cs₂CO₃ (175 mg, 0.53 mmol, 1.0 equiv) in DMF (6.0 mL) at 0° C. under nitrogen atmosphere. To the above mixture was added prop-2-en-1-yl (2S,3S,4S,5R,6S)-6-[4-({[chloromethyl(methyl)carbamoyl]oxy}methyl)-2-nitrophenoxy]-3,4,5-tris({[(prop-2-en-1-yloxy)carbonyl]oxy})oxane-2-carboxylate (400 mg, 0.53 mmol, 1.0 equiv) in portions over 30 min at 0° C. The resulting mixture was stirred for 30 min at 0° C. under nitrogen atmosphere. LCMS indicated the reaction was completed. The reaction mixture was purified by reverse flash chromatography with the following conditions: column, C18 silica gel; mobile phase, ACN in water (0.1% FA), 10% to 100% gradient in 30 min; detector, UV 254 nm. The resulting mixture was concentrated under vacuum to afford prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)carbamoyl}oxy)methyl]-2-nitrophenoxy}-3,4,5-tris({[(prop-2-en-1-yloxy)carbonyl]oxy})oxane-2-carboxylate (Compound 41-12, 310 mg, 38%) as a solid. LCMS: (ES, m/s): 1334 [M+H]⁺, 1234 [M+H−100]⁺.

### Step 12. Synthesis of Compound 41-13

To a stirred solution of prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)carbamoyl}oxy)methyl]-2-nitrophenoxy}-3,4,5-tris({[(prop-2-en-1-yloxy)carbonyl]oxy})oxane-2-carboxylate (Compound 41-12, 430 mg, 0.32 mmol, 1.0 equiv) in methanol (8.0 mL) were added AcOH (8.0 mL) and Zn (210 mg, 3.22 mmol, 10.00 equiv) in portions at room temperature under nitrogen atmosphere. The resulting mixture was stirred for overnight at room temperature under nitrogen atmosphere. LCMS indicated the reaction was completed. The resulting mixture was concentrated under vacuum. The residue was purified by reverse flash chromatography with the following conditions: column, C18 silica gel; mobile phase ACN in water (0.05% TFA), 10% to 80% gradient in 30 min; detector, UV 254 nm. The collected fraction was lyophilized to afford prop-2-en-1-yl (2S,3 S,4S,5R,6S)-6-{2-amino-4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)carbamoyl}oxy)methyl]phenoxy}-3,4,5-tris({[(prop-2-en-1-yloxy)carbonyl]oxy})oxane-2-carboxylate (Compound 41-13, 360 mg, 77%) as a green solid. LCMS: (ES, m/s): 1304 [M+H]⁺, 653 [M/2+H]⁺, 1326 [M+Na]⁺.

### Step 13. Synthesis of Compound 41-14

To a stirred solution of 3-[3-(2,5-dioxopyrrol-1-yl)propanamido]propanoic acid (Compound 41-13, 110 mg, 0.46 mmol, 1.5 equiv) and HATU (175 mg, 0.46 mmol, 1.5 equiv) in DMF (6.0 mL) were added prop-2-en-1-yl (2S,3S,4S,5R,6S)-6-(2-amino-4-{[({[3-(5-{[({3-chloro-4-[2-(2-{methyl[(prop-2-en-1-yloxy)carbonyl]amino}ethoxy)ethyl]phenyl}carbamoyl)amino]methyl}-1-oxo-3H-isoindol-2-yl)-2,6-dioxopiperidin-1-yl]methyl}(methyl)carbamoyl)oxy]methyl}phenoxy)-3,4,5-tris({[(prop-2-en-1-yloxy)carbonyl]oxy})oxane-2-carboxylate (750 mg, 0.58 mmol, 1.0 equiv) and DIEA (118 mg, 0.92 mmol, 3.0 equiv) in portions at room temperature under nitrogen atmosphere. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase ACN in water (0.05% TFA), 10% to 70% gradient in 30 min; detector, UV 254 nm and 220 nm. The resulting mixture was lyophilized to afford prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)carbamoyl}oxy)methyl]-2-{3-[3-(2,5-dioxopyrrol-1-yl)propanamido]propanamido}phenoxy}-3,4,5-tris({[(prop-2-en-1-yloxy)carbonyl]oxy})oxane-2-carboxylate (Compound 41-14, 300 mg, 57%) as a white solid. LCMS: (ES, m/s): 1527 [M+H]⁺, 1427 [M+H−100]⁺, 1549 [M+Na]⁺.

### Step 14. Synthesis of Compound 41-15

To a stirred solution of prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)carbamoyl}oxy)methyl]-2-{3-[3-(2,5-dioxopyrrol-1-yl)propanamido]propanamido}phenoxy}-3,4,5-tris({[(prop-2-en-1-yloxy)carbonyl]oxy})oxane-2-carboxylate (Compound 41-14, 200 mg, 0.13 mmol, 1.0 equiv) in THE (20 mL) was added TEA (53 mg, 0.52 mmol, 4.0 equiv), formic acid (18 mg, 0.39 mmol, 3.00 equiv), Pd(PPh₃)₄ (60 mg, 0.05 mmol, 0.4 equiv) in portions at room temperature under nitrogen atmosphere. The resulting mixture was stirred for 4 h at room temperature under nitrogen atmosphere. LCMS indicated the reaction was completed. The resulting mixture was concentrated under vacuum. The crude product was used in the next step directly without further purification. LCMS: (ES, m/s): 1134 [M+H−100]⁺, 1234 [M+H]⁺, 1256 [M+Na]⁺.

### Step 15. Synthesis of Compound (XLI)

To a stirred solution of (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](methyl)carbamoyl}oxy)methyl]-2-{3-[3-(2,5-dioxopyrrol-1-yl)propanamido]propanamido}phenoxy}-3,4,5-trihydroxyoxane-2-carboxylic acid (200 mg, 0.16 mmol, 1.0 equiv) in DCM (5.0 mL) was added TFA (1.0 mL) dropwise at 0° C. under nitrogen atmosphere. The resulting mixture was stirred for 2 h at room temperature under nitrogen atmosphere. LCMS indicated the reaction was completed. The reaction mixture was concentrated to dryness under vacuum. The residue was purified by reverse flash chromatography with the following conditions: column, C18 silica gel; mobile phase, ACN in water (0.1% FA), 0% to 50% gradient in 30 min; detector, UV 254 nm. The collected fraction was lyophilized to dryness to give the crude product. The crude product was re-purified by Prep-HPLC with the following conditions Column: XBridge Prep OBD C18 Column, 19*250 mm, 5 m; Mobile Phase A: Water (0.1% FA), Mobile Phase B: ACN; Flow rate: 25 mL/min; Gradient: 24% B to 44% B in 7 min, 44% B; Wave Length: 254 nm; RT1 (min): 4.63; The collected fraction was lyophilized to afford (2S,3S,4S,5R,6S)-6-[4-({[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)(methyl)carbamoyl]oxy}methyl)-2-{3-[3-(2,5-dioxopyrrol-1-yl)propanamido]propanamido}phenoxy]-3,4,5-trihydroxyoxane-2-carboxylic acid (Compound (LXI), 7.3 mg, 3%) as a white solid. LCMS: (ES, m/s): 1134 [M+H]⁺, 568 [M/2+H]⁺; ¹H NMR (300 MHz, DMSO-d₆) δ 10.7-10.5 (m, 1H), 9.17 (s, 1H), 8.90-8.30 (m, 1H), 8.15-8.10 (m, 2H), 7.73-7.68 (m, 2H), 7.65-7.40 (m, 2H), 7.35-7.00 (m, 4H), 6.97 (s, 2H), 5.78 (br s, 1H), 5.40-4.75 (m, 6H), 4.70-4.00 (m, 5H), 3.61-3.48 (m, 8H), 3.25-3.20 (m, 3H), 3.06-3.00 (m, 3H), 2.97-2.70 (m, 7H), 2.58 (s, 3H), 2.40-2.20 (m, 3H), 2.08-1.88 (m, 1H).

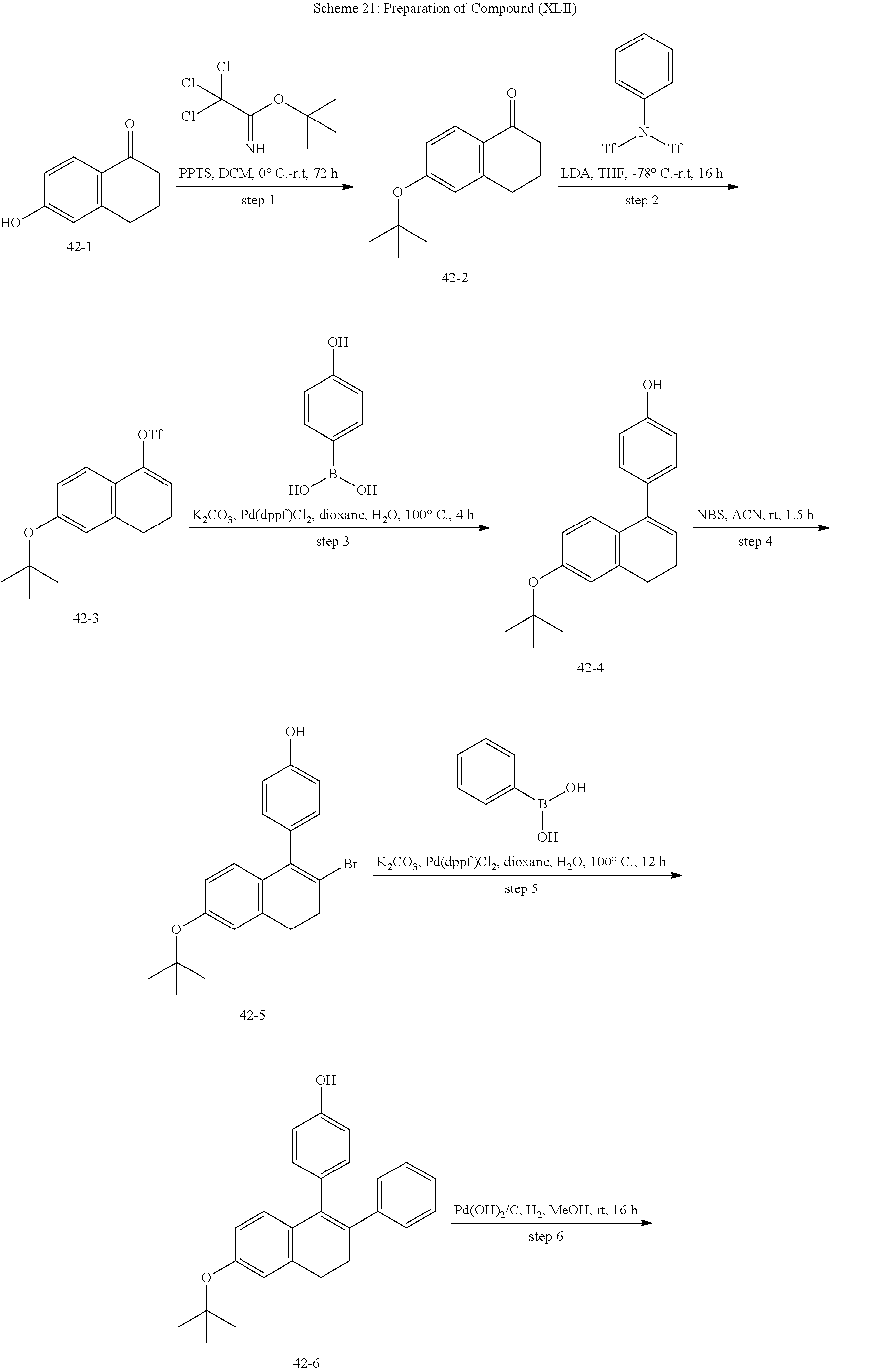

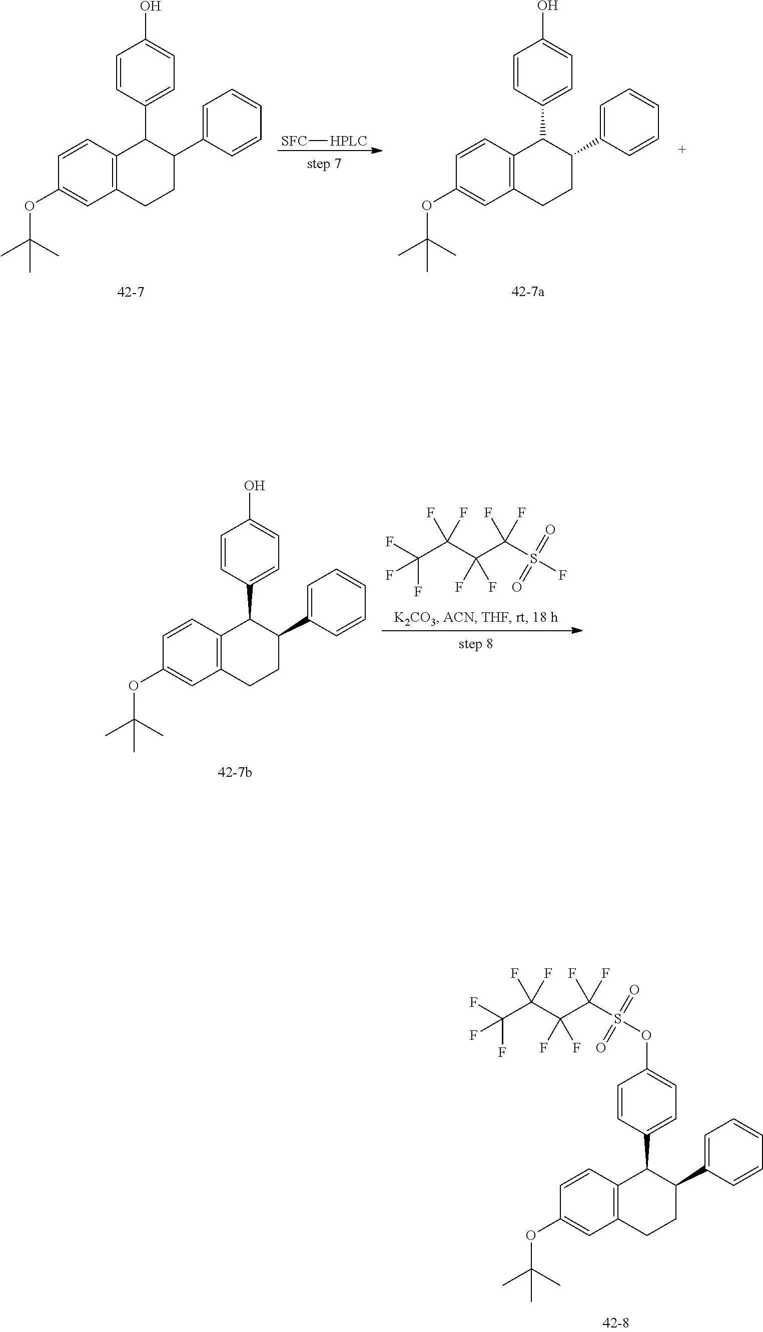

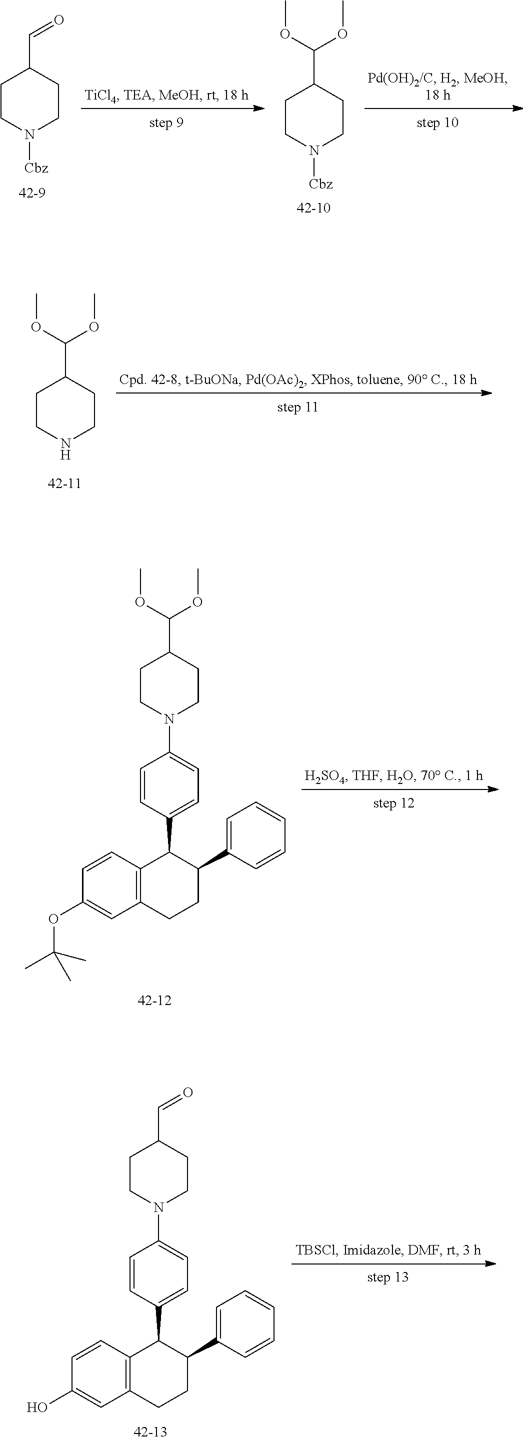 [SMILES: O=CC1CCN(C(=O)OCc2ccccc2)CC1 ; COC(OC)C1CCN(C(=O)OCc2ccccc2)CC1 ; COC(OC)C1CCNCC1 ; COC(OC)C1CCN(c2ccc([C@@H]3c4ccc(OC(C)(C)C)cc4CC[C@@H]3c3ccccc3)cc2)CC1 ; O=CC1CCN(c2ccc([C@@H]3c4ccc(O)cc4CC[C@@H]3c3ccccc3)cc2)CC1]

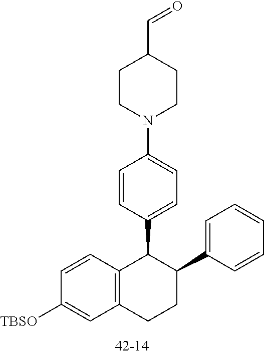 [SMILES: CC(C)(C)[Si](C)(C)Oc1ccc2c(c1)CC[C@H](c1ccccc1)[C@@H]2c1ccc(N2CCC(C=O)CC2)cc1]

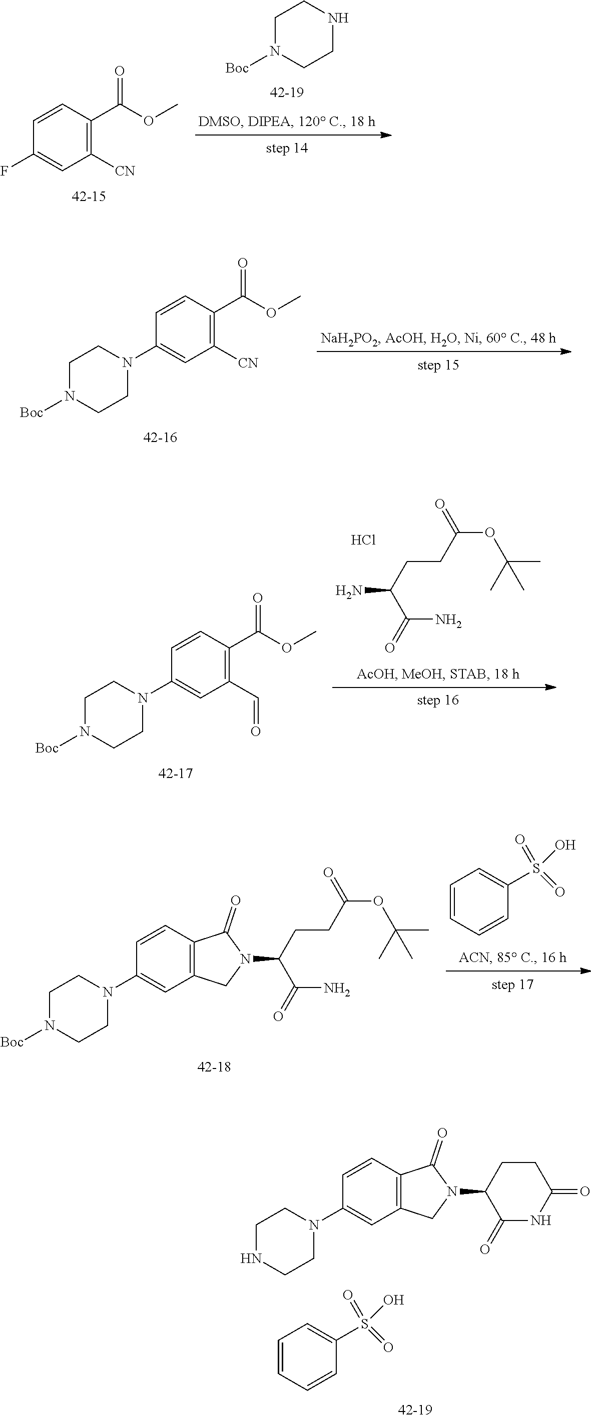

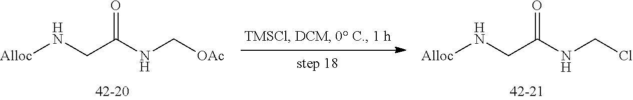

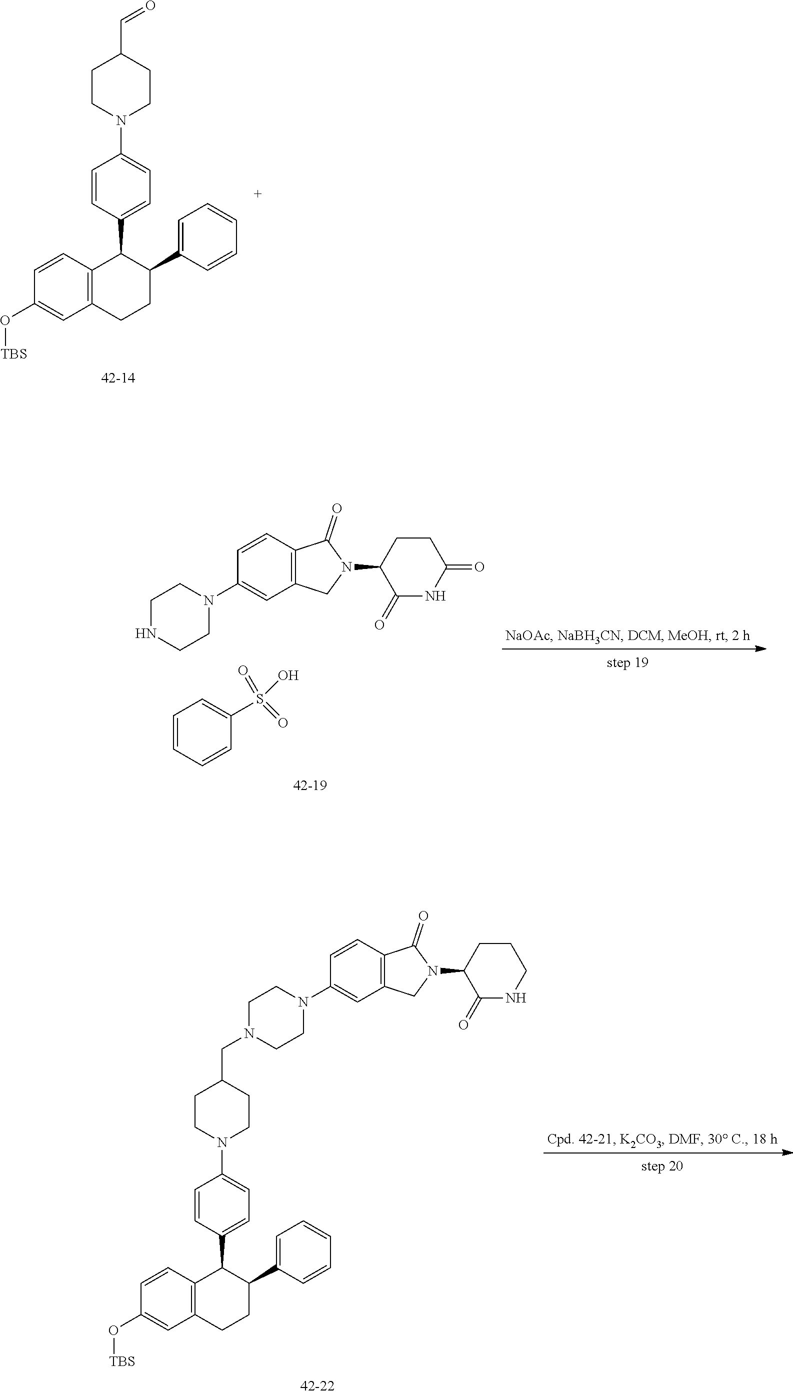

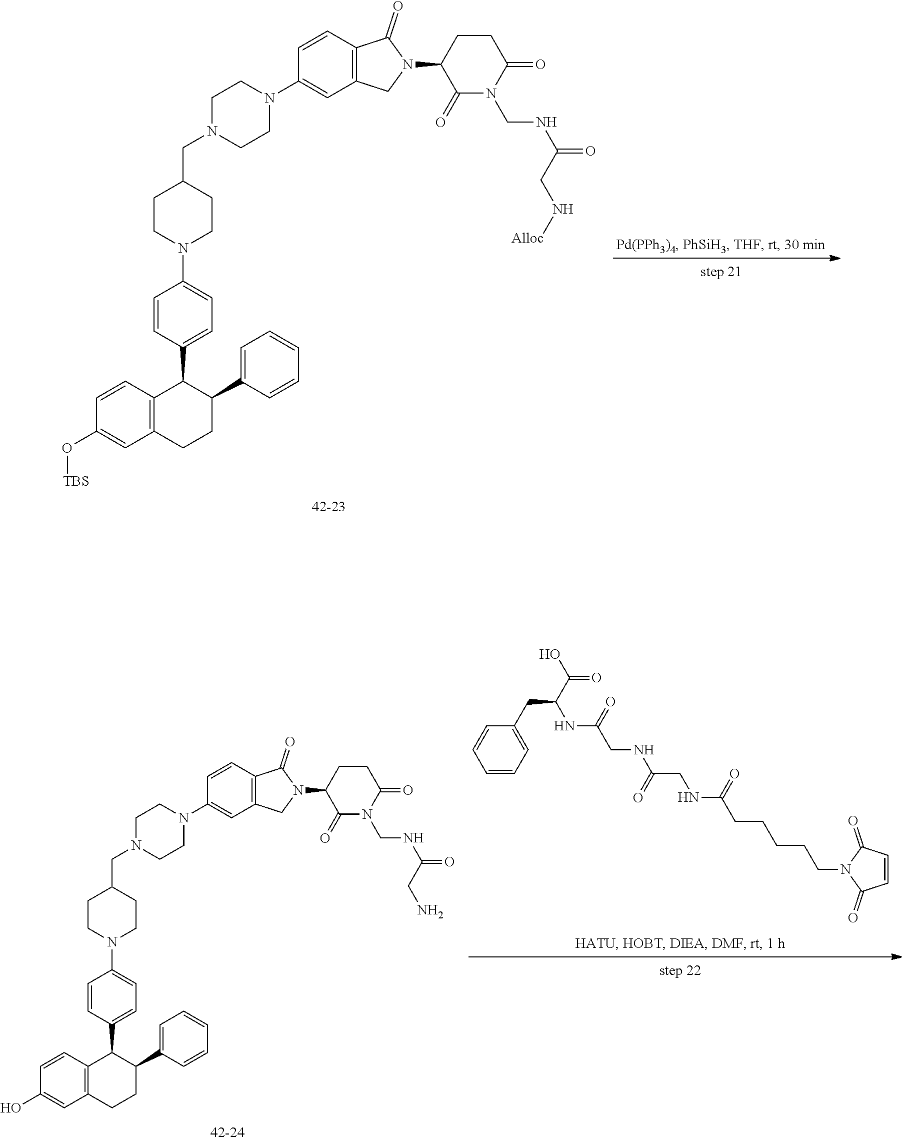

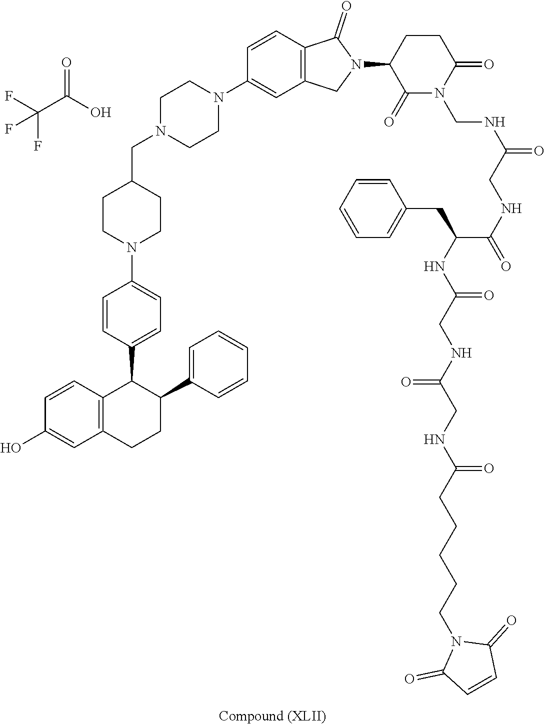 [SMILES: O=C(O)C(F)(F)F ; O=C(CCCCCN1C(=O)C=CC1=O)NCC(=O)NCC(=O)N[C@@H](Cc1ccccc1)C(=O)NCC(=O)NCN1C(=O)CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C1=O]

### Step 1. Synthesis of Compound 42-2

To a stirred mixture of 6-hydroxy-3,4-dihydro-2H-naphthalen-1-one (Compound 42-1, 30 g, 184.97 mmol, 1 equiv) and tert-butyl-2,2,2-trichloroethanimidoate (40.42 g, 184.97 mmol, 1 equiv) in DCM (1 L) was added PPTS (4.65 g, 18.50 mmol, 0.1 equiv) in portions at 0° C. The resulting mixture was stirred for 72 h at 25° C. LCMS indicated the reaction was completed. The resulting mixture was concentrated under reduced pressure and residue was purified by silica gel column chromatography, eluted with PE/EA (1:1) to afford 6-(tert-butoxy)-3,4-dihydro-2H-naphthalen-1-one (Compound 42-2, 9 g, 22%) as a yellow oil. LCMS (ES, m/z): 219 [M+H]⁺.

### Step 2. Synthesis of Compound 42-3

To a stirred mixture of 6-(tert-butoxy)-3,4-dihydro-2H-naphthalen-1-one (Compound 42-2, 10 g, 45.81 mmol, 1 equiv) in THE (150 mL) was added LDA (9 mL, 1.5 equiv, 68.71 mmol, 2.0 M in THF) dropwise at −78° C. under nitrogen atmosphere. The resulting mixture was stirred for 1 h at −78° C. under nitrogen atmosphere. To the above mixture was added 1,1,1-trifluoro-N-phenyl-N-trifluoromethanesulfonylmethanesulfonamide (19.6 g, 54.87 mmol, 1.20 equiv) at −78° C. The resulting mixture was stirred for additional 16 h at 25° C. LCMS indicated the reaction was completed. The resulting mixture was quenched by the addition of sat. NH₄Cl (aq.) at room temperature and extracted with EtOAc (3×200 mL). The combined organic layers were washed with brine (200 mL), dried over anhydrous Na₂SO₄, filtered and concentrated under reduced pressure. The residue was purified by silica gel column chromatography, eluted with PE/EA (5:1) to afford 6-(tert-butoxy)-3,4-dihydronaphthalen-1-yl trifluoromethanesulfonate (Compound 42-3, 9.0 g, 56%) as a yellow oil. LCMS: (ES. m/z): 351 [M+H]⁺; ¹H-NMR (300 MHz, CDCl₃): 7.45 (d, J=8.4 Hz, 1H), 6.91-6.86 (m, 1H), 6.71-6.66 (m, 1H), 5.94 (t, J=4.8 Hz, 1H), 2.86-2.81 (m, 2H), 2.54-2.48 (m, 2H), 1.38 (s, 9H).

### Step 3. Synthesis of Compound 42-4

A mixture of 6-(tert-butoxy)-3,4-dihydronaphthalen-1-yl trifluoromethanesulfonate (Compound 42-3, 16 g, 45.67 mmol, 1 equiv), 4-hydroxyphenylboronic acid (7.56 g, 54.80 mmol, 1.2 equiv), K₂CO₃ (12.62 g, 91.34 mmol, 2 equiv) and Pd(dppf)Cl₂ (3.34 g, 4.57 mmol, 0.1 equiv) in dioxane (250 mL) and H₂O (50 mL) was stirred for 4 h at 100° C. under nitrogen atmosphere. LCMS indicated the reaction was completed. The mixture was allowed to cool down to room temperature. The resulting mixture was quenched with water (200 mL) and extracted with EtOAc (3×200 mL). The combined organic layers were washed with brine (200 mL), dried over anhydrous Na₂SO₄, filtered and concentrated under reduced pressure. The residue was purified by silica gel column chromatography, eluted with PE/EA (3:1) to afford 4-[6-(tert-butoxy)-3,4-dihydronaphthalen-1-yl]phenol (Compound 42-4, 11 g, 82%) as a yellow solid. LCMS (ES, m/z): 295 [M+H]⁺; ¹H-NMR (400 MHz, CDCl₃): 7.27-7.16 (m, 2H), 7.00-6.92 (m, 1H), 6.91-6.83 (m, 3H), 6.76-6.74 (m, 1H), 5.97 (t, J=4.8 Hz, 1H), 2.82-2.80 (m, 2H), 2.39-2.35 (m, 2H), 1.40 (s, 9H).

### Step 4. Synthesis of Compound 42-5

To a stirred mixture of 4-[6-(tert-butoxy)-3,4-dihydronaphthalen-1-yl]phenol (Compound 42-4, 11 g, 37.36 mmol, 1 equiv) in ACN (250 mL) was added NBS (5.99 g, 33.63 mmol, 0.9 equiv) at room temperature. The reaction was stirred for 1.5 h at 25° C. LCMS indicated the reaction was completed. The resulting mixture was concentrated under reduced pressure. The and residue was purified by silica gel column chromatography, eluted with PE/EA (3:1) to afford 4-[2-bromo-6-(tert-butoxy)-3,4-dihydronaphthalen-1-yl]phenol (Compound 42-5, 9 g, 64%) as a yellow oil. LCMS (ES, m/z): 373 [M+H]⁺; ¹H-NMR (400 MHz, CDCl₃): 7.21-7.00 (m, 2H), 6.98-6.87 (m, 2H), 6.85-6.74 (m, 1H), 6.68-6.64 (m, 1H), 6.59 (d, J=8.4 Hz, 1H), 3.08-2.82 (m, 4H), 1.37 (s, 9H).

### Step 5. Preparation of Compound 42-6

A mixture of 4-[2-bromo-6-(tert-butoxy)-3,4-dihydronaphthalen-1-yl]phenol (Compound 42-5, 9 g, 24.11 mmol, 1 equiv), phenyl boronic acid (3.09 g, 25.32 mmol, 1.05 equiv), K₂CO₃ (6.66 g, 48.22 mmol, 2 equiv) and Pd(dppf)Cl₂ (1.76 g, 2.41 mmol, 0.1 equiv) in dioxane (100 mL) and H₂O (20 mL) was stirred for 12 h at 100° C. under nitrogen atmosphere. LCMS indicated the reaction was completed. The mixture was allowed to cool down to room temperature and extracted with EtOAc (3×100 mL). The combined organic layers were washed with brine (60 mL), dried over anhydrous Na₂SO₄, filtered and concentrated under reduced pressure. The residue was purified by silica gel column chromatography, eluted with PE/EA (3:1) to afford 4-[6-(tert-butoxy)-2-phenyl-3,4-dihydronaphthalen-1-yl]phenol (Compound 42-6, 6 g, 67%) as a yellow oil. LCMS (ES, m/z): 371 [M+H]⁺; ¹H-NMR (400 MHz, CDCl₃): 7.24-7.15 (m, 2H), 7.10-7.01 (m, 3H), 6.98-6.92 (m, 2H), 6.87-6.85 (m, 1H), 6.76-6.68 (m, 4H), 2.95-2.91 (m, 2H), 2.83-2.80 (m, 2H), 1.39 (s, 9H).

### Step 6. Preparation of Compound 42-7

To a solution of 4-[6-(tert-butoxy)-2-phenyl-3,4-dihydronaphthalen-1-yl]phenol (Compound 42-6, 6 g, 16.20 mmol, 1 equiv) in MeOH (200 mL) was added Pd(OH)₂/C (20%, 1.8 g) under nitrogen atmosphere in a 500 mL round-bottom flask. The mixture was hydrogenated at room temperature for 16 h under hydrogen atmosphere using a hydrogen balloon. LCMS indicated the reaction was completed. The mixture was filtered through a diatomaceous earth (Celite™) pad and concentrated under reduced pressure. The residue was purified by silica gel column chromatography, eluted with PE/EA (1:1) to afford 4-[6-(tert-butoxy)-2-phenyl-1,2,3,4-tetrahydronaphthalen-1-yl]phenol (Compound 42-7, 4 g, 66%) as a yellow solid. LCMS (ES, m/z): 371 [M−H]⁻.

### Step 7. Preparation of Compounds 42-7a and 42-7b

4-[6-(tert-Butoxy)-2-phenyl-1,2,3,4-tetrahydronaphthalen-1-yl]phenol (Compound 42-7, 3.5 g) was separated by SFC-HPLC with the following condition: Column: CHIRALPAK IG, 3*25 cm, 5 μm; Mobile Phase A: CO₂, Mobile Phase B: MEOH (0.1% 2M NH₃-MEOH); Flow rate: 70 mL/min; Gradient: isocratic 35% B; Column Temperature (° C.): 35; Back Pressure (bar): 100; Wave Length: 220 nm; RT1 (min): 3.08; RT2 (min): 3.94; Sample Solvent: MeOH:DCM=1:2; Injection Volume: 1.8 mL; The second eluting isomer (RT2=3.94 min) was concentrated to dryness to afford 4-[(1R,2S)-6-(tert-butoxy)-2-phenyl-1,2,3,4-tetrahydronaphthalen-1-yl]phenol (Compound 42-7b, 1.5 g). LCMS (ES, m/z): 371 [M−H]; ¹H-NMR (300 MHz, CDCl₃): 7.15-7.10 (m, 3H), 6.89-6.67 (m, 5H), 6.43-6.30 (m, 2H), 6.28-6.15 (m, 2H), 4.24-4.20 (m, 1H), 3.36-3.34 (m, 1H), 3.05-3.01 (m, 2H), 2.28-2.05 (m, 1H), 1.93-1.73 (m, 1H), 1.37 (s, 9H).

### Step 8. Preparation of Compound 42-8

The mixture of 4-[(1R,2S)-6-(tert-butoxy)-2-phenyl-1,2,3,4-tetrahydronaphthalen-1-yl]phenol (Compound 42-7, 1.8 g, 4.83 mmol, 1 equiv) and perfluorobutanesulfonyl fluoride (1.46 g, 4.83 mmol, 1.00 equiv) in ACN (10 mL) and THF (10 mL) was stirred for overnight at room temperature under nitrogen atmosphere. LCMS indicated the reaction was completed. The resulting mixture was concentrated under vacuum. The residue was purified by silica gel column chromatography, eluted with PE/EA (10:1) to afford 4-[(1R,2S)-6-(tert-butoxy)-2-phenyl-1,2,3,4-tetrahydronaphthalen-1-yl]phenyl 1,1,2,2,3,3,4,4,4-nonafluorobutane-1-sulfonate (Compound 42-8, 2.1 g, 59%) as a colorless oil. LCMS: (ES, m/z): 655 [M+H]⁺; ¹H-NMR (400 MHz, CDCl₃): 7.18-7.10 (m, 3H), 6.97-6.87 (m, 3H), 6.85-6.74 (m, 4H), 6.54-6.37 (m, 2H), 4.35-4.31 (m, 1H), 3.49-3.45 (m, 1H), 3.19-2.98 (m, 2H), 2.21-2.09 (m, 1H), 1.96-1.84 (m, 1H), 1.40 (s, 9H).

### Step 9. Preparation of Compound 42-10

To a stirred solution of benzyl 4-formylpiperidine-1-carboxylate (Compound 42-9, 10 g, 40.43 mmol, 1.0 equiv) in MeOH (20 mL) was added TiCl₄ (0.38 g, 2.02 mmol, 0.05 equiv) in DCM=2.2 mL in portions at room temperature under nitrogen atmosphere. To the above mixture was added TEA (0.41 g, 4.04 mmol, 0.1 equiv) in portions over 30 min at room temperature. The resulting mixture was stirred for additional overnight at room temperature. LCMS indicated the reaction was completed. The resulting mixture was concentrated under vacuum. The residue was purified by silica gel column chromatography, eluted with PE/EA (1:1) to afford benzyl 4-(dimethoxymethyl)piperidine-1-carboxylate (Compound 42-10, 11 g, 83%) as an oil. LCMS: (ES, m/z): 294 [M+H]⁺; ¹H-NMR (300 MHz, CDCl₃): 7.47-7.26 (m, 5H), 5.14 (s, 2H), 1.20-4.16 (m, 2H), 4.04 (d, J=6.6 Hz, 1H), 3.37 (s, 6H), 2.756-2.73 (m, 2H), 1.85-1.66 (m, 3H), 1.41-1.04 (m, 2H).

### Step 10. Preparation of Compound 42-11

To a stirred solution of benzyl 4-(dimethoxymethyl)piperidine-1-carboxylate (Compound 42-10, 10 g, 34.08 mmol, 1.0 equiv) in MeOH (100 mL) was added Pd(OH)₂/C (0.96 g, 20%) in portions at room temperature under hydrogen atmosphere. The resulting mixture was stirred for overnight at room temperature under hydrogen atmosphere. LCMS indicated the reaction was completed. The resulting mixture was filtered, the filter cake was washed with MeOH (2×5 mL). The filtrate was concentrated under reduced pressure to afford 4-(dimethoxymethyl)piperidine (5.6 g, 100%) as a colorless oil. The crude product was used in the next step directly without further purification. LCMS: (ES, m/z): 160 [M+H]⁺; ¹H-NMR (300 MHz, CDCl₃): 4.13-3.88 (m, 3H), 3.35 (s, 6H), 3.19-3.16 (m, 2H), 2.63-2.60 (m, 2H), 1.76-1.73 (m, 3H), 1.47-1.12 (m, 2H).

### Step 11. Preparation of Compound 42-12

To a stirred mixture of 4-[(1R,2S)-6-(tert-butoxy)-2-phenyl-1,2,3,4-tetrahydronaphthalen-1-yl]phenyl 1,1,2,2,3,3,4,4,4-nonafluorobutane-1-sulfonate (Compound 42-8, 500 mg, 0.76 mmol, 1 equiv) and 4-(dimethoxymethyl)piperidine (Compound 42-11, 175 mg, 1.10 mmol, 1.44 equiv) in toluene (5 mL) were added Xphos (76 mg, 0.16 mmol, 0.21 equiv) and Pd(OAc)₂ (26 mg, 0.11 mmol, 0.15 equiv) in portions at room temperature under nitrogen atmosphere. The resulting mixture was stirred for overnight at 90° C. under nitrogen atmosphere. 30% desired product was detected by LCMS. The mixture was allowed to cool down to room temperature. The resulting mixture was concentrated under reduced pressure. The residue was purified by silica gel column chromatography, eluted with PE/EA (8:1) to afford 1-{4-[(1R,2S)-6-(tert-butoxy)-2-phenyl-1,2,3,4-tetrahydronaphthalen-1-yl]phenyl}-4-(dimethoxymethyl)piperidine (Compound 42-12, 300 mg, 76%) as a yellow solid. LCMS: (ES, m/z): 514 [M+H]⁺

### Step 12. Preparation of Compound 42-13

A mixture of 1-{4-[(1R,2S)-6-(tert-butoxy)-2-phenyl-1,2,3,4-tetrahydronaphthalen-1-yl]phenyl}-4-(dimethoxymethyl)piperidine (Compound 42-12, 1 g, 1.94 mmol, 1 equiv) in H₂SO₄ (20 mL) and THE (20 mL) was stirred for 1 h at 70° C. under nitrogen atmosphere. LCMS indicated the reaction was completed. The mixture was basified to pH 8 with saturated NaHCO₃(aq.). The resulting mixture was extracted with EtOAc (3×40 mL). The combined organic layers were washed with brine (40 mL), dried over anhydrous Na₂SO₄, filtered and concentrated under reduced pressure. This resulted in 1-{4-[(1R,2S)-6-hydroxy-2-phenyl-1,2,3,4-tetrahydronaphthalen-1-yl]phenyl}piperidine-4-carbaldehyde (Compound 42-13, 650 mg, 81%) as a white solid. LCMS (ES, m/z): 412 [M+H]⁺.

### Step 13. Preparation of Compound 42-14

To a stirred solution of 1-{4-[(1R,2S)-6-hydroxy-2-phenyl-1,2,3,4-tetrahydronaphthalen-1-yl]phenyl}piperidine-4-carbaldehyde (Compound 42-13, 350 mg, 0.85 mmol, 1 equiv) in DMF (8 mL) was added TBSCl (153 mg, 1.02 mmol, 1.2 equiv) and Imidazole (174 mg, 2.55 mmol, 3 equiv) dropwise at room temperature under nitrogen atmosphere. The resulting mixture was stirred for 3 h at room temperature under nitrogen atmosphere. LCMS indicated the reaction was completed. The reaction was quenched with water at room temperature and extracted with EtOAc (3×15 mL). The combined organic layers were washed with brine (3×10 mL), dried over anhydrous Na₂SO₄, filtered and concentrated under reduced pressure. This resulted in 1-{4-[(1R,2S)-6-[(tert-butyldimethylsilyl)oxy]-2-phenyl-1,2,3,4-tetrahydronaphthalen-1-yl]phenyl}piperidine-4-carbaldehyde (380 mg, 84%) as a white solid. LCMS (ES, m/z): 526 [M+H]⁺.

### Step 14. Preparation of Compound 42-16

To a stirred mixture of methyl 2-cyano-4-fluorobenzoate (Compound 42-15, 10 g, 55.82 mmol, 1 equiv) and tert-butyl piperazine-1-carboxylate (Compound 42-19, 12.5 g, 67.11 mmol, 1.20 equiv) in DMSO (100 mL) was added DIEA (28.5 g, 220.51 mmol, 3.95 equiv) dropwise at room temperature. The resulting mixture was stirred for overnight at 120° C. under nitrogen atmosphere. LCMS indicated the reaction was completed. The mixture was allowed to cool down to room temperature, diluted with water (500 mL) and extracted with EtOAc (3×500 mL). The combined organic layers were washed with brine (500 mL), dried over anhydrous Na₂SO₄, filtered and concentrated under reduced pressure. The residue was purified by silica gel column chromatography, eluted with PE/EA (1:1) to afford tert-butyl 4-[3-cyano-4-(methoxycarbonyl)phenyl]piperazine-1-carboxylate (Compound 42-16, 12 g, 59%) as a yellow solid. LCMS: (ES, m/z): 346 [M+H]⁺; ¹HNMR (400 MHz, DMSO-d₆) δ 7.91 (d, J=8.8 Hz, 1H), 7.43 (d, J=2.8 Hz, 1H), 7.22 (dd, J=8.8, 2.8 Hz, 1H), 3.83 (s, 3H), 3.59-3.38 (m, 8H), 1.42 (s, 9H).

### Step 15. Preparation of Compound 42-17

To a stirred mixture of tert-butyl 4-[3-cyano-4-(methoxycarbonyl)phenyl]piperazine-1-carboxylate (Compound 42-16, 10 g, 28.95 mmol, 1 equiv) and AcOH (10 mL, 174.51 mmol, 6.03 equiv) in H₂O (10 mL) was added sodium hypophosphite (25.50 g, 289.84 mmol, 10.01 equiv) and Raney-Ni (7.49 g, 87.42 mmol, 3.02 equiv) in portions at room temperature under nitrogen atmosphere. The resulting mixture was stirred for 48 h at 60° C. under nitrogen atmosphere. LCMS indicated ˜50% of product and ˜30% starting material remained. The mixture was allowed to cool down to room temperature and concentrated under vacuum. The reaction was quenched with water/ice at room temperature and extracted with EtOAc (3×100 mL). The combined organic layers were washed with brine (100 mL), dried over anhydrous Na₂SO₄, filtered and concentrated under reduced pressure. The residue was purified by silica gel column chromatography, eluted with PE/EA (1:1) to afford tert-butyl 4-[3-formyl-4-(methoxycarbonyl)phenyl]piperazine-1-carboxylate (Compound 42-17, 4.8 g, 43%) as a yellow solid. LCMS: (ES, m/z): 349 [M+H]⁺.

### Step 16. Preparation of Compound 42-18

To a stirred mixture of tert-butyl 4-[3-formyl-4-(methoxycarbonyl)phenyl]piperazine-1-carboxylate (Compound 42-17, 6.0 g, 17.22 mmol, 1 equiv) and tert-butyl (4S)-4-amino-4-carbamoylbutanoate hydrochloride (4.93 g, 20.67 mmol, 1.20 equiv) in MeOH (170 mL) were added AcOH (1.50 g, 24.97 mmol, 1.45 equiv) and STAB (14.42 g, 68.03 mmol, 3.95 equiv) at room temperature under nitrogen atmosphere. The resulting mixture was stirred for overnight at room temperature under nitrogen atmosphere. LCMS indicated the reaction was completed. The mixture was neutralized to pH 7 with saturated NaHCO₃(aq.) and extracted with EtOAc (3×50 mL). The combined organic layers were washed with brine (60 mL), dried over anhydrous Na₂SO₄, filtered and concentrated under reduced pressure. The residue was purified by silica gel column chromatography, eluted with PE/EA (1:1) to afford tert-butyl 4-{2-[(1S)-4-(tert-butoxy)-1-carbamoyl-4-oxobutyl]-1-oxo-3H-isoindol-5-yl}piperazine-1-carboxylate (Compound 42-18, 1.5 g, 17%) as a yellow solid. LCMS (ES, m/z): 503 [M+H]⁺.

### Step 17. Preparation of Compound 42-19

A mixture of tert-butyl 4-{2-[(1S)-4-(tert-butoxy)-1-carbamoyl-4-oxobutyl]-1-oxo-3H-isoindol-5-yl}piperazine-1-carboxylate (Compound 42-18, 1.5 g, 2.98 mmol, 1 equiv) and benzenesulfonic acid (0.94 g, 5.97 mmol, 2 equiv) in ACN (20 mL) was stirred for 16 h at 85° C. LCMS indicated the reaction was completed. The mixture was allowed to cool down to room temperature and concentrated under reduced pressure. The residue was purified by trituration with EtOAc (10 mL). This resulted in (3S)-3-[1-oxo-5-(piperazin-1-yl)-3H-isoindol-2-yl]piperidine-2,6-dione; benzenesulfonic acid (1.3 g, 89%) as a yellow solid. LCMS (ES, m/z): 329 [M+H]⁺.

### Step 18. Preparation of Compound 42-21

To a stirred solution of (2-{[(prop-2-en-1-yloxy)carbonyl]amino}acetamido)methyl acetate (Compound 42-20, 500 mg, 2.17 mmol, 1 equiv) in DCM was added TMSCl (944 mg, 8.69 mmol, 4 equiv) dropwise at 0° C. under nitrogen atmosphere. The resulting mixture was stirred for 1 h at 0° C. under nitrogen atmosphere. LCMS indicated the reaction was completed. The resulting mixture was concentrated under reduced pressure. The crude product mixture was used in the next step directly without further purification.

### Step 19. Preparation of Compound 42-22

To a stirred solution of (3S)-3-[1-oxo-5-(piperazin-1-yl)-3H-isoindol-2-yl]piperidine-2,6-dione; benzenesulfonic acid (Compound 42-19, 422 mg, 0.87 mmol, 1.2 equiv) in DCM (7 mL) and MeOH (7 mL) was added NaOAc (119 mg, 1.44 mmol, 2 equiv) dropwise at room temperature under nitrogen atmosphere. The resulting mixture was stirred for 0.5 h at room temperature under nitrogen atmosphere. To the above mixture was added 1-{4-[(1R,2S)-6-[(tert-butyldimethylsilyl)oxy]-2-phenyl-1,2,3,4-tetrahydronaphthalen-1-yl]phenyl}piperidine-4-carbaldehyde (Compound 42-14, 380 mg, 0.72 mmol, 1 equiv) and NaBH₃CN (136 mg, 2.17 mmol, 3 equiv) dropwise at room temperature and resulting mixture was stirred for additional 2 h at room temperature. LCMS indicated the reaction was completed. The resulting mixture was concentrated under reduced pressure. The residue was purified by silica gel column chromatography, eluted with CH₂Cl₂/MeOH (10:1) to afford (3S)-3-(5-{4-[(1-{4-[(1R,2S)-6-[(tert-butyldimethylsilyl)oxy]-2-phenyl-1,2,3,4-tetrahydronaphthalen-1-yl]phenyl}piperidin-4-yl)methyl]piperazin-1-yl}-1-oxo-3H-isoindol-2-yl)piperidine-2,6-dione (Compound 42-22, 255 mg, 42%) as a white solid. LCMS (ES, m/z): 824 [M+H]⁺.

### Step 20. Preparation of Compound 42-23

To a stirred mixture of (3S)-3-(5-{4-[(1-{4-[(1R,2S)-6-[(tert-butyldimethylsilyl)oxy]-2-phenyl-1,2,3,4-tetrahydronaphthalen-1-yl]phenyl}piperidin-4-yl)methyl]piperazin-1-yl}-1-oxo-3H-isoindol-2-yl)piperidine-2,6-dione (Compound 42-22, 250 mg, 0.29 mmol, 1 equiv) in DMF (5 mL) was added K₂CO₃ (82 mg, 0.59 mmol, 2 equiv) dropwise at room temperature under nitrogen atmosphere. The resulting mixture was stirred for 30 min at room temperature under nitrogen atmosphere. To the above mixture was added prop-2-en-1-yl N-[(chloromethylcarbamoyl)methyl]carbamate (Compound 42-21, 123 mg, 0.59 mmol, 2 equiv) dropwise at room temperature and resulting mixture was stirred for additional overnight at 30° C. LCMS indicated the reaction was completed. The mixture was concentrated to dryness under vacuum. The residue was purified by reverse flash chromatography with the following conditions: column, C18 silica gel; mobile phase, ACN in water (0.05% TFA), 10% to 60% gradient in 30 min; detector, UV 254 nm. This resulted in prop-2-en-1-yl N-[({[(3S)-3-(5-{4-[(1-{4-[(1R,2S)-6-[(tert-butyldimethylsilyl)oxy]-2-phenyl-1,2,3,4-tetrahydronaphthalen-1-yl]phenyl}piperidin-4-yl)methyl]piperazin-1-yl}-1-oxo-3H-isoindol-2-yl)-2,6-dioxopiperidin-1-yl]methyl}carbamoyl)methyl]carbamate (60 mg, 20%) as a white solid. LCMS (ES, m/z): 1008 [M+H]⁺.

### Step 21. Preparation of Compound 42-24

To a stirred solution of prop-2-en-1-yl N-[({[(3S)-3-(5-{4-[(1-{4-[(1R,2S)-6-[(tert-butyldimethylsilyl)oxy]-2-phenyl-1,2,3,4-tetrahydronaphthalen-1-yl]phenyl}piperidin-4-yl)methyl]piperazin-1-yl}-1-oxo-3H-isoindol-2-yl)-2,6-dioxopiperidin-1-yl]methyl}carbamoyl)methyl]carbamate (Compound 42-23, 50 mg, 0.050 mmol, 1 equiv) and Pd(PPh₃)₄ (10 mg, 0.009 mmol, 0.17 equiv) in THE (50 mL) was added phenylsilane (10 mg, 0.092 mmol, 1.86 equiv) in portions at room temperature under nitrogen atmosphere. The resulting mixture was stirred for 30 min at room temperature under nitrogen atmosphere. Desired product could be detected by LCMS. The residue was purified by reverse flash chromatography with the following conditions: column, C18 silica gel; mobile phase, MeCN in Water (0.1% TFA), 5% to 50% gradient in 30 min; detector, UV 254 nm. This resulted in 2-amino-N-{[(3S)-3-(5-{4-[(1-{4-[(1R,2S)-6-[(tert-butyldimethylsilyl)oxy]-2-phenyl-1,2,3,4-tetrahydronaphthalen-1-yl]phenyl}piperidin-4-yl)methyl]piperazin-1-yl}-1-oxo-3H-isoindol-2-yl)-2,6-dioxopiperidin-1-yl]methyl}acetamide (15 mg, 32.73%) as a white solid, LCMS (ES, m/z): 924 [M+H]⁺ and 2-amino-N-{[(3S)-3-(5-{4-[(1-{4-[(1R,2S)-6-hydroxy-2-phenyl-1,2,3,4-tetrahydronaphthalen-1-yl]phenyl}piperidin-4-yl)methyl]piperazin-1-yl}-1-oxo-3H-isoindol-2-yl)-2,6-dioxopiperidin-1-yl]methyl}acetamide (Compound 42-24, 20 mg, 49%) as a white solid. LCMS (ES, m/z): 810 [M+H]⁺.

### Step 22. Preparation of Compound (XLII)

To a stirred mixture of (2S)-2-(2-{2-[6-(2,5-dioxopyrrol-1-yl)hexanamido]acetamido}acetamido)-3-phenylpropanoic acid (Compound 40-6, 10 mg, 0.021 mmol, 1.01 equiv) and HATU (10 mg, 0.026 mmol, 1.25 equiv) in DMF (2 mL) were added HOBT (3 mg, 0.022 mmol, 1.06 equiv), 2-amino-N-{[(3S)-3-(5-{4-[(1-{4-[(1R,2S)-6-hydroxy-2-phenyl-1,2,3,4-tetrahydronaphthalen-1-yl]phenyl}piperidin-4-yl)methyl]piperazin-1-yl}-1-oxo-3H-isoindol-2-yl)-2,6-dioxopiperidin-1-yl]methyl}acetamide (Compound 42-24, 17 mg, 0.021 mmol, 1.0 equiv) and DIEA (10 mg, 0.077 mmol, 3.69 equiv) at room temperature under nitrogen atmosphere. The resulting mixture was stirred for 1 h at room temperature under nitrogen atmosphere. LCMS indicated the reaction was completed. The reaction mixture was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 25% B to 40% B in 10 min, 40% B; Wave Length: 254 nm; RT1 (min): 9.38; The collected fraction was lyophilized to afford 6-(2,5-dioxopyrrol-1-yl)-N-{[({[(1S)-1-{[({[(3 S)-3-(5-{4-[(1-{4-[(1R,2S)-6-hydroxy-2-phenyl-1,2,3,4-tetrahydronaphthalen-1-yl]phenyl}piperidin-4-yl)methyl]piperazin-1-yl}-1-oxo-3H-isoindol-2-yl)-2,6-dioxopiperidin-1-yl]methyl}carbamoyl)methyl]carbamoyl}-2-phenylethyl]carbamoyl}methyl)carbamoyl]methyl}hexanamide; trifluoroacetic acid (Compound (XLII), 8.5 mg, 29%) as a white solid. LCMS (ES, m/z): 1265 [M+H−TFA]⁺, 633 [M/2+H-TFA]⁺; ¹H-NMR (300 MHz, DMSO-d₆): 9.37 (br s, 1H), 9.14 (br s, 1H), 8.23-8.08 (m, 5H), 7.60-7.58 (m, 1H), 7.25-7.23 (m, 4), 7.17-7.14 (m, 5H), 7.10 (s, 1H), 6.99-6.97 (m, 3H), 6.85-6.83 (m, 2H), 6.65-6.61 (m, 3H), 6.45-6.42 (m, 1H), 6.25-6.23 (m, 1H), 5.15-4.90 (m, 3H), 4.50-4.41 (m, 1H), 4.47-4.44 (m, 1H), 4.28-4.15 (m, 2H), 4.05-3.95 (m, 2H), 3.80-3.65 (m, 5H), 3.60-3.56 (m, 3H), 3.38-3.31 (m, 4H), 3.25-2.90 (m, 10H), 2.85-2.75 (m, 2H), 2.70-2.61 (m, 2H), 2.35-2.31 (m, 1H), 2.15-2.08 (m, 3H), 2.05-1.90 (m, 2H), 1.85-1.70 (m, 3H), 1.47-1.45 (m, 4H), 1.35-1.12 (m, 5H).

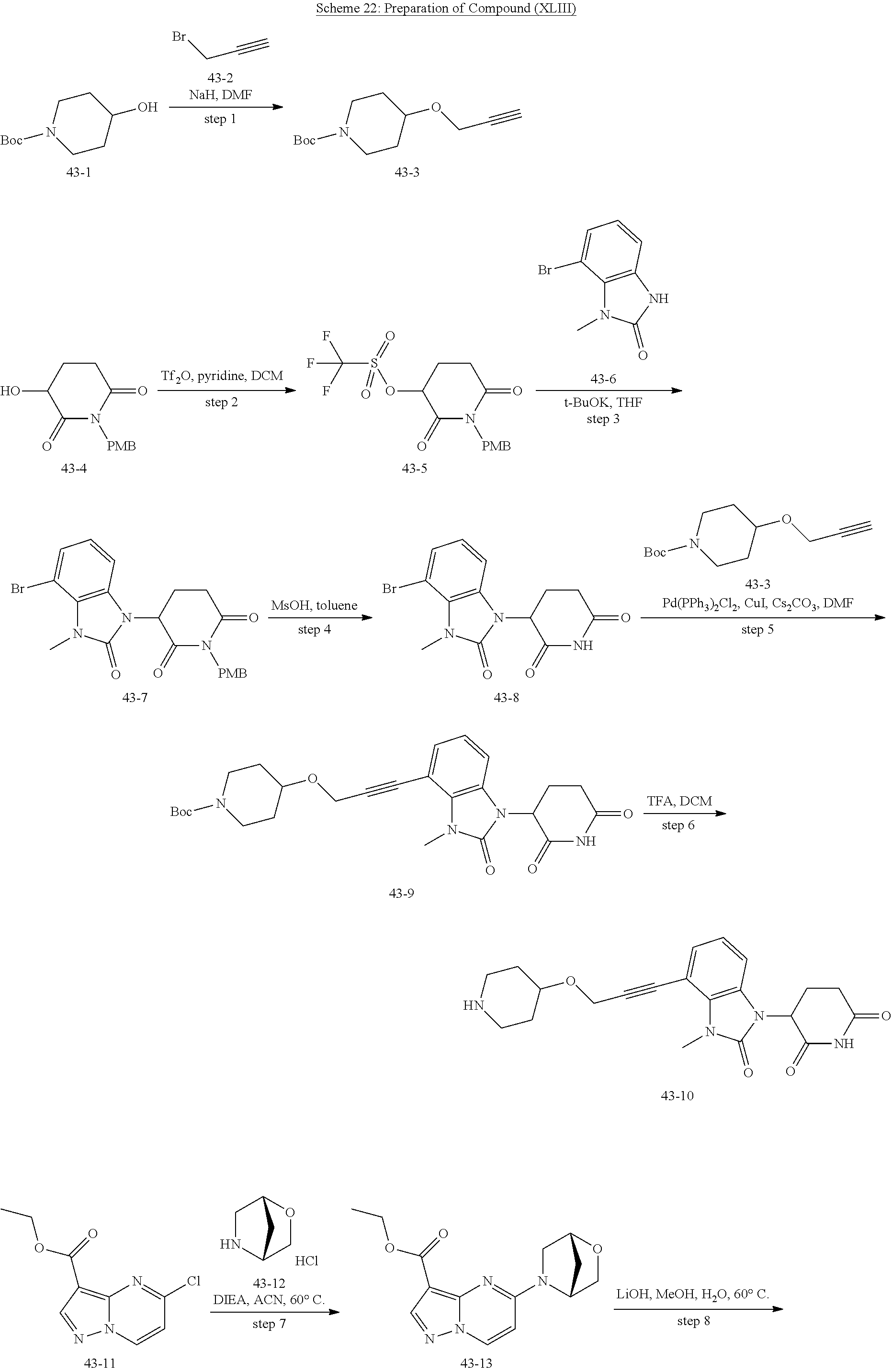

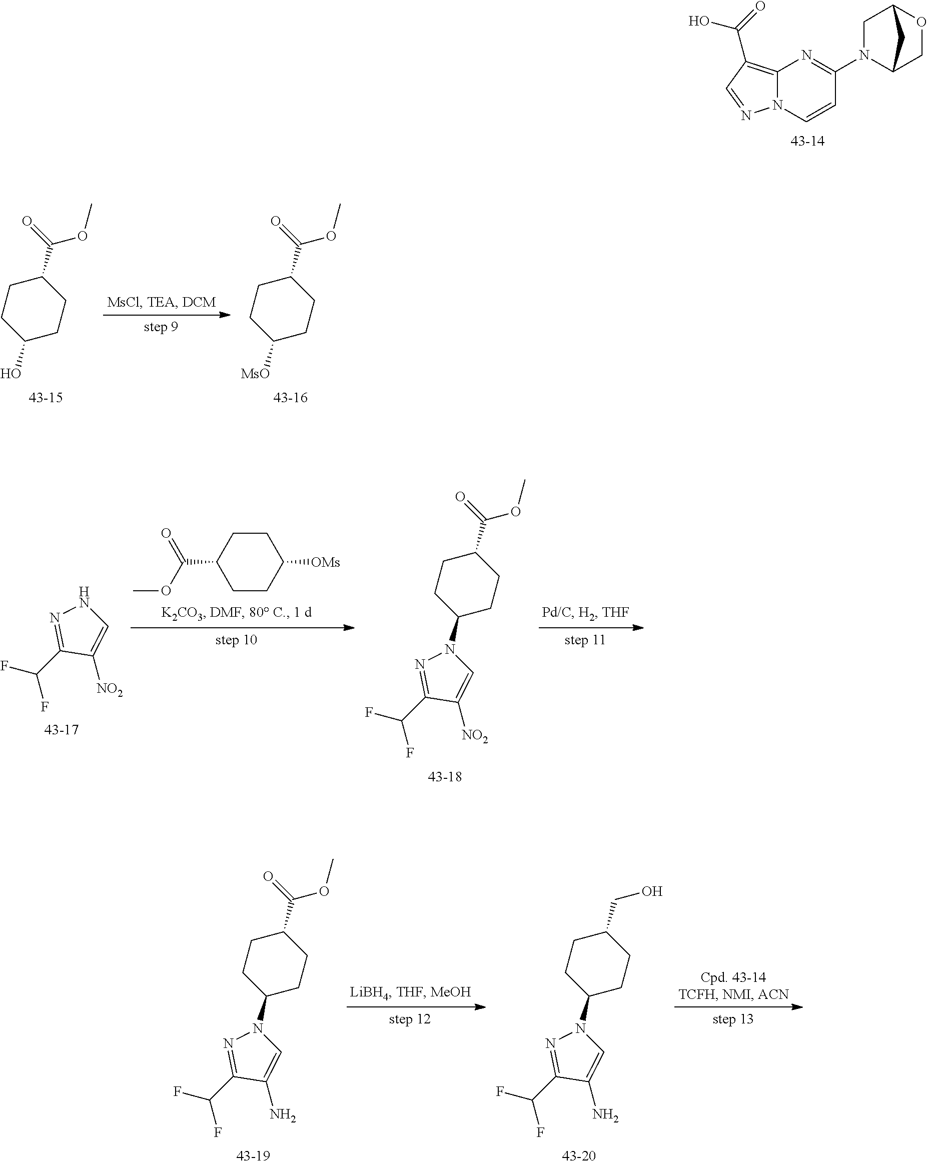

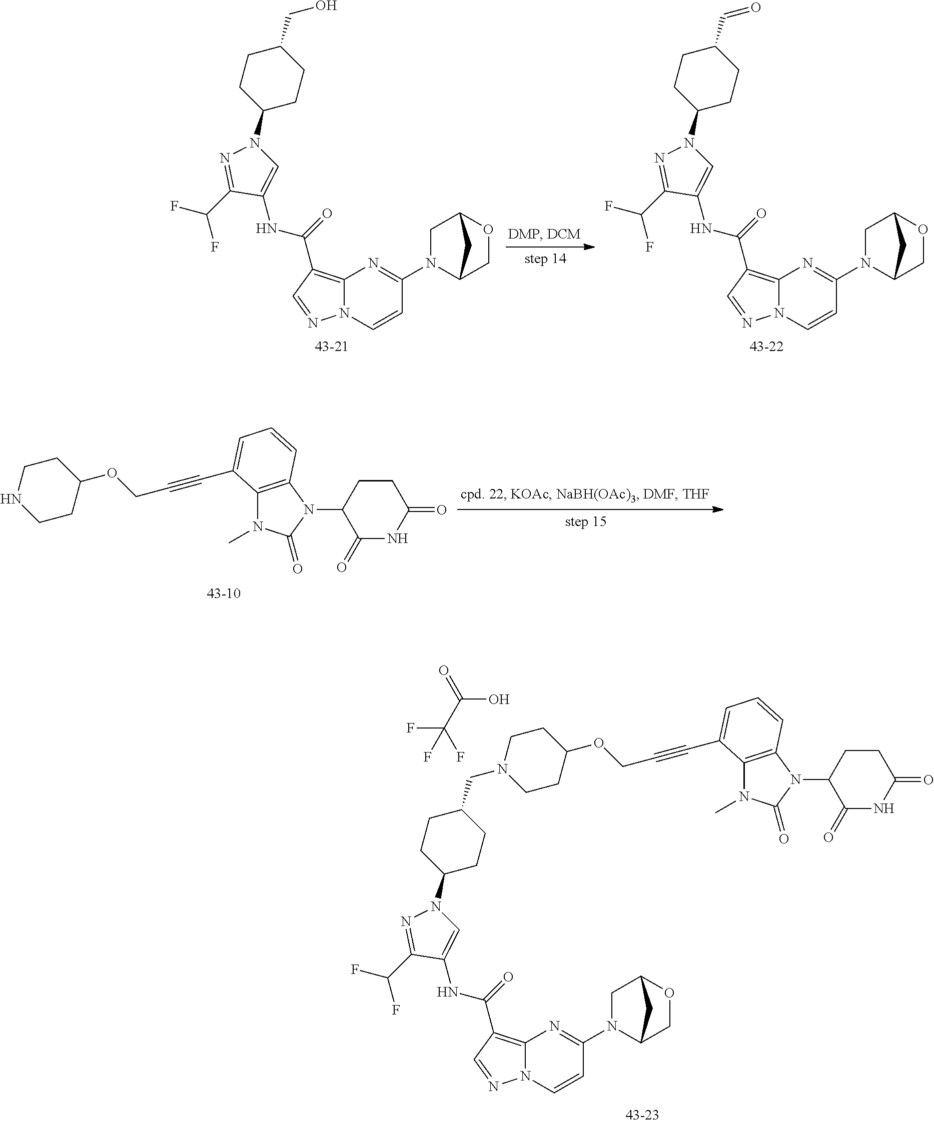

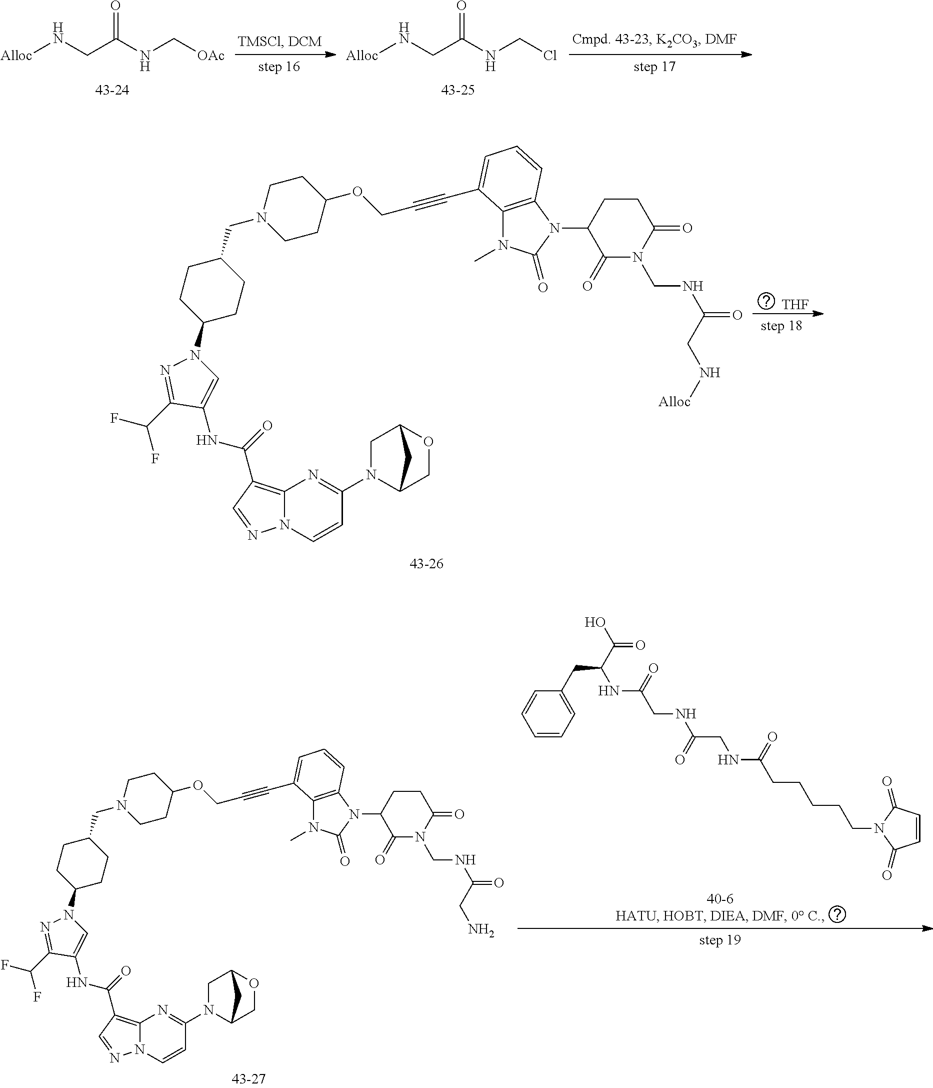

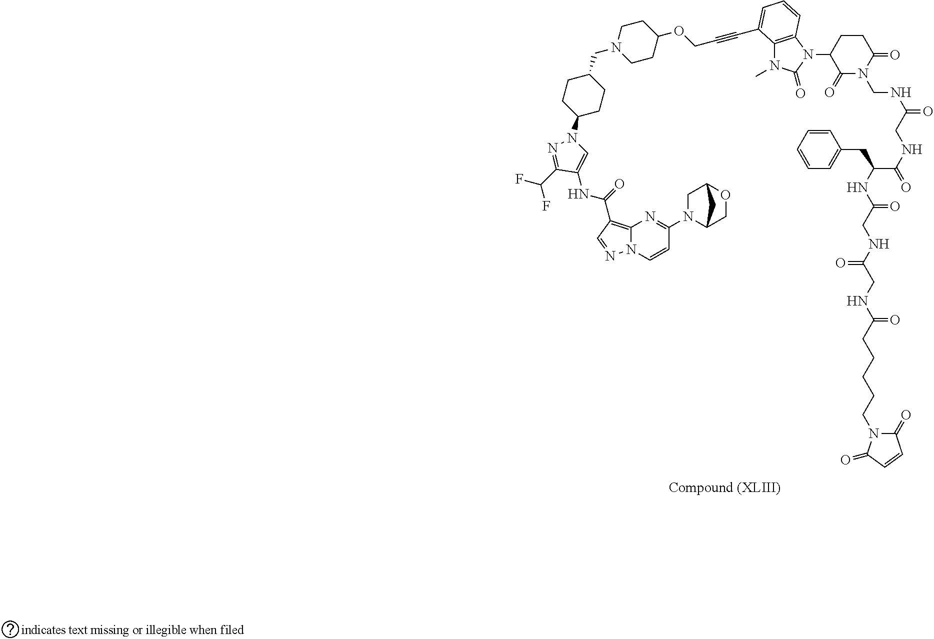 [SMILES: Cn1c(=O)n(C2CCC(=O)N(CNC(=O)CNC(=O)[C@H](Cc3ccccc3)NC(=O)CNC(=O)CNC(=O)CCCCCN3C(=O)C=CC3=O)C2=O)c2cccc(C#CCOC3CCN(C[C@H]4CC[C@H](n5cc(NC(=O)c6cnn7ccc(N8C[C@H]9C[C@@H]8CO9)nc67)c(C(F)F)n5)CC4)CC3)c21]

### Step 1. Synthesis of Compound 43-3

A mixture of tert-butyl 4-hydroxypiperidine-1-carboxylate (Compound 43-1, 1.00 g, 4.96 mmol, 1 equiv) in DMF (5 mL) was treated with NaH (0.24 g, 5.96 mmol, 1.2 equiv, 60%) in portions at 0° C. The resulting mixture was stirred at 0° C. for 30 min under nitrogen atmosphere. Then propargyl bromide (Compound 43-2, 0.59 g, 4.96 mmol, 1 equiv) was added into the above mixture dropwise. The resulting mixture was stirred for another 2 h. TLC indicated the reaction was completed. The reaction was quenched by the addition of water (10 mL) at 0° C. The resulting mixture was extracted with EA (3×10 mL). The combined organic layers were washed with brine (15 ml), dried over anhydrous sodium sulfate. After filtration, the filtrate was concentrated under reduced pressure. The residue was purified by silica gel column chromatography, eluted with PE/EA (3:1) to afford tert-butyl 4-(prop-2-yn-1-yloxy)piperidine-1-carboxylate (Compound 43-3, 350 mg, 29%) as a yellow oil. LCMS (ESI, m/z): 240 [M+H]⁺.

### Step 2. Preparation of Compound 43-5

To a stirred mixture of 3-hydroxy-1-[(4-methoxyphenyl)methyl]piperidine-2,6-dione (Compound 43-4, 6.00 g, 24.07 mmol, 1 equiv) and pyridine (3.81 g, 48.14 mmol, 2 equiv) in DCM (240 mL) was added (trifluoromethane)sulfonyl trifluoromethanesulfonate (10.18 g, 36.10 mmol, 1.5 equiv) dropwise at 0° C. under nitrogen atmosphere. The resulting mixture was stirred at 0° C. for 1.5 h. LCMS indicated the reaction was completed. The solvent was removed under reduced pressure. The residue was purified by silica gel column chromatography, eluted with PE/EA (3:1) to afford 1-[(4-methoxyphenyl)methyl]-2,6-dioxopiperidin-3-yl trifluoromethanesulfonate (Compound 43-5, 7.2 g, 78%) as a white solid. LCMS (ESI, m/z): 404 [M+Na]⁺.

### Step 3. Preparation of Compound 43-7

A mixture of 7-bromo-1-methyl-3H-1,3-benzodiazol-2-one (Compound 43-6, 2.98 g, 13.11 mmol, 1 equiv) and potassium tert-butoxide (1.77 g, 15.73 mmol, 1.2 equiv) in THE (170 mL) was stirred for 30 min at 0° C. under nitrogen atmosphere. 1-[(4-methoxyphenyl)methyl]-2,6-dioxopiperidin-3-yl trifluoromethanesulfonate (Compound 43-5, 5.00 g, 13.11 mmol, 1 equiv) in THE (30 mL) was added into the above mixture dropwise at 0° C. The resulting mixture was stirred for 30 min at 25° C. LCMS indicated the reaction was completed. The reaction was quenched by addition of ammonium chloride solution (100 mL). The resulting mixture was extracted with EA (3×200 mL). The combined organic layers were dried over anhydrous sodium sulfate. After filtration, the filtrate was concentrated under reduced pressure. The residue was purified by reverse phase flash with the following conditions: column, C18 silica gel, 120 g, 20-35 um; mobile phase, water with 0.5% NH₄HCO₃ and ACN (0% to 100% gradient in 50 min); detector, UV 254 nm. The eluent was concentrated to afford 3-(4-bromo-3-methyl-2-oxo-1,3-benzodiazol-1-yl)-1-[(4-methoxyphenyl)methyl]piperidine-2,6-dione (Compound 43-7, 1.1 g, 18%) as a white solid. LCMS (ESI, m/z): 458,460 [M+H]⁺.

### Step 4. Preparation of Compound 43-8

A mixture of 3-(4-bromo-3-methyl-2-oxo-1,3-benzodiazol-1-yl)-1-[(4-methoxyphenyl)methyl]piperidine-2,6-dione (Compound 43-7, 4.00 g, 8.72 mmol, 1 equiv) and CH₃SO₃H (11.33 mL, 174.56 mmol, 20 equiv) in toluene (30 mL) was stirred for 2 h at 120° C. LCMS indicated the reaction was completed. The reaction mixture was allowed to cool down to room temperature. The solvent was removed under reduced pressure. The residue was purified by reverse flash chromatography with the following conditions: column, C18 silica gel, 80 g, 20-35 um; mobile phase, water with 0.1% NH₄HCO₃ and ACN (5% to 95% gradient in 50 min); detector, UV 254 nm. The eluent was concentrated to give 3-(4-bromo-3-methyl-2-oxo-1,3-benzodiazol-1-yl)piperidine-2,6-dione (Compound 43-8, 1.5 g, 50%) as a white solid. LCMS (ESI, m/z): 338,340 [M+H]⁺.

### Step 5. Preparation of Compound 43-9

A mixture of 3-(4-bromo-3-methyl-2-oxo-1,3-benzodiazol-1-yl)piperidine-2,6-dione (Compound 43-8, 1.5 g, 4.43 mmol, 1 equiv), tert-butyl 4-(prop-2-yn-1-yloxy)piperidine-1-carboxylate (Compound 43-3, 1.59 g, 6.65 mmol, 1.5 equiv), dichloropalladium; bis(triphenylphosphane) (0.62 g, 0.88 mmol, 0.2 equiv), CuI (0.17 g, 0.88 mmol, 0.2 equiv) and cesium carbonate (5.78 g, 17.74 mmol, 4 equiv) in DMF (35 mL) was stirred for 2 h at 80° C. under nitrogen atmosphere. LCMS indicated the reaction was completed. The reaction mixture was allowed to cool down to room temperature. The reaction was quenched with water (50 mL). The resulting mixture was extracted with EA (3×50 mL). The combined organic layers were washed with brine (50 mL), dried over anhydrous sodium sulfate. After filtration, the filtrate was concentrated under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The eluent was concentrated to afford tert-butyl 4-({3-[1-(2,6-dioxopiperidin-3-yl)-3-methyl-2-oxo-1,3-benzodiazol-4-yl]prop-2-yn-1-yl}oxy)piperidine-1-carboxylate (Compound 43-9, 700 mg, 31%) as a white solid. LCMS (ESI, m/z): 497 [M+H]⁺.

### Step 6. Preparation of Compound 43-10

To a stirred mixture of tert-butyl 4-({3-[1-(2,6-dioxopiperidin-3-yl)-3-methyl-2-oxo-1,3-benzodiazol-4-yl]prop-2-yn-1-yl}oxy)piperidine-1-carboxylate (Compound 43-9, 700 mg, 1.41 mmol, 1 equiv) in DCM (20 mL) was added TFA (4 mL). The resulting mixture was stirred for 1 h at 25° C. LCMS indicated the reaction was completed. The solvent was removed under reduced pressure to afford crude 3-{3-methyl-2-oxo-4-[3-(piperidin-4-yloxy)prop-1-yn-1-yl]-1,3-benzodiazol-1-yl}piperidine-2,6-dione (Compound 43-10, 800 mg, crude) as a yellow oil. LCMS (ESI, m/z): 397 [M+H]⁺.

### Step 7. Preparation of Compound 43-13

To a stirred mixture of ethyl 5-chloropyrazolo[1,5-a]pyrimidine-3-carboxylate (Compound 43-11, 10.00 g, 44.32 mmol, 1 equiv) and (1R,4R)-2-oxa-5-azabicyclo[2.2.1]heptane (6.59 g, 66.48 mmol, 1.5 equiv) in ACN (200.00 mL) was added DIEA (22.91 g, 177.28 mmol, 4 equiv) at 0° C. under air atmosphere. The resulting mixture was stirred for 1 h at 60° C. under air atmosphere. LCMS indicated the reaction was completed. The reaction mixture was allowed to cool down to room temperature. The resulting mixture was diluted with water (400 mL). The resulting mixture was extracted with EtOAc (3×400 mL). The combined organic layers were washed with brine (3×100 mL), dried over anhydrous Na₂SO₄. After filtration, the filtrate was concentrated under reduced pressure. This resulted in ethyl 5-[(1R,4R)-2-oxa-5-azabicyclo[2.2.1]heptan-5-yl]pyrazolo[1,5-a]pyrimidine-3-carboxylate (Compound 43-13, 10 g, 78%) as a yellow solid. LCMS: (ES·m/z): 289 [M+H]⁺.

### Step 8. Preparation of Compound 43-14

To a stirred solution of ethyl 5-[(1R,4R)-2-oxa-5-azabicyclo[2.2.1]heptan-5-yl]pyrazolo[1,5-a]pyrimidine-3-carboxylate (Compound 43-13, 10 g, 34.68 mmol, 1 equiv) in MeOH (100 mL) was added H₂O (20 mL) and LiOH (4.15 g, 173.42 mmol, 5 equiv) in portions at 0° C. under air atmosphere. The resulting mixture was stirred for overnight at 60° C. under air atmosphere. LCMS indicated the reaction was completed. The reaction mixture was allowed to cool down to room temperature. The resulting mixture was concentrated under reduced pressure. The residue was purified by reverse flash chromatography with the following conditions: column, C18 silica gel; mobile phase, TFA in Water (0.05% TFA), 0% to 50% gradient in 30 min; detector, UV 254 nm. This resulted in 5-[(1R,4R)-2-oxa-5-azabicyclo[2.2.1]heptan-5-yl]pyrazolo[1,5-a]pyrimidine-3-carboxylic acid (Compound 43-14, 3.7 g, 40%) as a yellow solid. LCMS: (ES·m/z): 261 [M+1]⁺.

### Step 9. Preparation of Compound 43-16

To a stirred mixture of methyl (1s,4s)-4-hydroxycyclohexane-1-carboxylate (Compound 43-15, 6.00 g, 37.92 mmol, 1 equiv) and triethylamine (1.15 g, 113.78 mmol, 3 equiv) in DCM (150 mL) was added methanesulfonyl chloride (5.21 g, 45.51 mmol, 1.2 equiv) dropwise at 0° C. under nitrogen atmosphere. The resulting mixture was stirred for 2 h at 0° C. TLC indicated the reaction was completed. The reaction was quenched by addition of waster (50 mL). The resulting mixture was extracted with DCM (3×150 mL). The combined organic layers were dried over anhydrous sodium sulfate. After filtration, the filtrate was concentrated under reduced pressure to afford methyl (1s,4s)-4-(methanesulfonyloxy)cyclohexane-1-carboxylate (Compound 43-16, 6.1 g, 68%) as a yellow oil. GCMS: (ES·m/z): 236[M]⁺.

### Step 10. Preparation of Compound 43-18

A mixture of 3-(difluoromethyl)-4-nitro-1H-pyrazole (Compound 43-17, 1.70 g, 10.42 mmol, 1 equiv), methyl (1s,4s)-4-(methanesulfonyloxy)cyclohexane-1-carboxylate (2.46 g, 10.42 mmol, 1 equiv) and K₂CO₃ (2.88 g, 20.84 mmol, 2 equiv) in DMF (25 mL) was stirred for 24 h at 80° C. LCMS indicated about 50% product produced. The solvent was removed under reduced pressure. The residue was quenched with water (25 mL). The resulting mixture was extracted with EA (3×30 mL). The combined organic layers were washed with brine (30 mL), dried over anhydrous sodium sulfate. After filtration, the filtrate was concentrated under reduced pressure. The residue was purified by silica gel column chromatography, eluted with PE/EA (3:1) to afford methyl (1r,4r)-4-[3-(difluoromethyl)-4-nitropyrazol-1-yl]cyclohexane-1-carboxylate (Compound 43-18, 1.1 g, 34%) as a yellow solid. LCMS (ESI, m/z): 304 [M+H]⁺.

### Step 11. Preparation of Compound 43-19

To a stirred mixture of methyl (1r,4r)-4-[3-(difluoromethyl)-4-nitropyrazol-1-yl]cyclohexane-1-carboxylate (Compound 43-18, 900 mg, 2.96 mmol, 1 equiv) in THE (30 mL) was added Pd/C (240 mg, 10%). The resulting mixture was stirred under hydrogen atmosphere for 4 h at 25° C. LCMS indicated the reaction was completed. The solid was filtered off. The filtrate was concentrated under reduced pressure to afford crude methyl (1r,4r)-4-[4-amino-3-(difluoromethyl)pyrazol-1-yl]cyclohexane-1-carboxylate (Compound 43-19, 890 mg, crude) as a white solid. LCMS (ESI, m/z): 274 [M+H]⁺.

### Step 12. Preparation of Compound 43-20

To a stirred mixture of methyl (1r,4r)-4-[4-amino-3-(difluoromethyl)pyrazol-1-yl]cyclohexane-1-carboxylate (Compound 43-19, 850 mg, 3.11 mmol, 1 equiv) in MeOH (3 mL) and THE (18 mL) was added LiBH₄ (3.10 mL, 6.22 mmol, 2 equiv) at 0° C. The resulting mixture was stirred for 2 h at 60° C. LCMS indicated the reaction was completed. The reaction was quenched by addition of water (10 mL) at 25° C. The resulting mixture was extracted with EA (3×20 mL). The combined organic layers were dried over anhydrous sodium sulfate. The residue was purified by silica gel column chromatography, eluted with DCM/MeOH (96:4) to afford [(1r,4r)-4-[4-amino-3-(difluoromethyl)pyrazol-1-yl]cyclohexyl]methanol (Compound 43-20, 550 mg, 72%) as a white solid. LCMS (ESI, m/z): 246 [M+H]⁺.

### Step 13. Preparation of Compound 43-21

A mixture of 5-[(1R,4R)-2-oxa-5-azabicyclo[2.2.1]heptan-5-yl]pyrazolo[1,5-a]pyrimidine-3-carboxylic acid (Compound 43-14, 230 mg, 0.89 mmol, 1 equiv), [chloro(dimethylamino)methylidene]dimethylazanium; hexafluoro-λ⁵-phosphanuide (300 mg, 1.07 mmol, 1.2 equiv) and 1-methyl-1H-imidazole (260 mg, 3.13 mmol, 3.5 equiv) in ACN (15 mL) was stirred for 30 min at 25° C. Then [(1r,4r)-4-[4-amino-3-(difluoromethyl)pyrazol-1-yl]cyclohexyl]methanol (Compound 43-20, 220 mg, 0.89 mmol, 1 equiv) was added into the above mixture. The resulting mixture was stirred for 2 h. LCMS indicated the reaction was completed. The solvent was removed under reduce pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The eluent was concentrated under reduced pressure to afford N-[3-(difluoromethyl)-1-[(1r,4r)-4-(hydroxymethyl)cyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-21, 200 mg, 45.74%) as a yellow solid. LCMS (ESI, m/z): 488 [M+H]⁺.

### Step 14. Preparation of Compound 43-22

To a stirred mixture of N-[3-(difluoromethyl)-1-[(1r,4r)-4-(hydroxymethyl)cyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-21, 1.00 g, 2.05 mmol, 1 equiv) in DCM (20 mL) was added 1,1-bis(acetyloxy)-3-oxo-3H-1λ⁵,2-benziodaoxol-1-yl acetate (960 mg, 2.25 mmol, 1.1 equiv). The resulting mixture was stirred for 1.5 h at 25° C. LCMS indicated the reaction was completed. The reaction was quenched with saturated sodium bicarbonate solution (15 mL). The resulting mixture was extracted with DCM (3×15 mL). The combined organic layers were dried over anhydrous sodium sulfate. After filtration, the filtrate was concentrated under reduced pressure. The residue was purified by silica gel column chromatography, eluted with DCM/MeOH (96:4) to afford N-[3-(difluoromethyl)-1-[(1r,4r)-4-formylcyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-22, 600 mg, 60%) as a yellow solid. LCMS (ESI, m/z): 486 [M+H]⁺.

### Step 15. Preparation of Compound 43-23

A mixture of N-[3-(difluoromethyl)-1-[(1r,4r)-4-formylcyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-22, 600 mg, 1.23 mmol, 1 equiv), 3-{3-methyl-2-oxo-4-[3-(piperidin-4-yloxy)prop-1-yn-1-yl]-1,3-benzodiazol-1-yl}piperidine-2,6-dione (Compound 43-10, 490 mg, 1.23 mmol, 1 equiv) and AcOK (240 mg, 2.47 mmol, 2 equiv) in DMF (3 mL) and THE (15 mL) was stirred for 30 min at 25° C. Then STAB (520 mg, 2.47 mmol, 2 equiv) was added into the above mixture. The resulting mixture was stirred for 1 h. LCMS indicated the reaction was completed. The solvent was removed under reduced pressure. The residue was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The eluent was concentrated to afford N-[3-(difluoromethyl)-1-[(1r,4r)-4-{[4-({3-[1-(2,6-dioxopiperidin-3-yl)-3-methyl-2-oxo-1,3-benzodiazol-4-yl]prop-2-yn-1-yl}oxy)piperidin-1-yl]methyl}cyclohexyl]pyrazol-4-yl]-5-[(1R,4R)-2-oxa-5-azabicyclo[2.2.1]heptan-5-yl]pyrazolo[1,5-a]pyrimidine-3-carboxamide (600 mg, 56%) as a yellow solid. LCMS (ES, m/z): 866 [M+H−TFA]⁺.

### Step 16. Preparation of Compound 43-25

A mixture of (2-{[(prop-2-en-1-yloxy)carbonyl]amino}acetamido)methyl acetate (Compound 43-24, 300 mg, 1.30 mmol, 1 equiv) and chlorotrimethylsilane (565 mg, 5.21 mmol, 4 equiv) in DCM (5 mL) was stirred for 1 h at 0° C. under nitrogen atmosphere. Analysis sample was quenched with MeOH and mass signal showed 203. LCMS indicated the reaction was completed. The solvent was removed under reduced pressure to afford crude prop-2-en-1-yl N-[(chloromethylcarbamoyl)methyl]carbamate (Compound 43-25, 350 mg, crude) as a white solid. The crude product was used to next step without any purification.

### Step 17. Preparation of Compound 43-26

A mixture of N-[3-(difluoromethyl)-1-[(1r,4r)-4-{[4-({3-[1-(2,6-dioxopiperidin-3-yl)-3-methyl-2-oxo-1,3-benzodiazol-4-yl]prop-2-yn-1-yl}oxy)piperidin-1-yl]methyl}cyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-23, 500 mg, 0.57 mmol, 1 equiv) and potassium carbonate (160 mg, 1.15 mmol, 2 equiv) in DMF (7.5 mL) was stirred for 20 min at 0° C. under nitrogen atmosphere. Then prop-2-en-1-yl N-[(chloromethylcarbamoyl)methyl]carbamate (Compound 43-25, 240 mg, 1.15 mmol, 2 equiv) was added into the above mixture. The resulting mixture was stirred for 1.5 h at 25° C. LCMS indicated the reaction was completed. The reaction system was purified by reverse flash chromatography with the following conditions: column, C18 silica gel, 80 g, 20-35 um; mobile phase, water with 0.1% TFA and ACN (0% to 100% gradient in 50 min); detector, UV 254 nm. The eluent was concentrated under reduced pressure to afford prop-2-en-1-yl N-[({[3-(3-methyl-2-oxo-4-{3-[(1-{[(1r,4r)-4-[3-(difluoromethyl)-4-{5-[(1R,4R)-2-oxa-5-azabicyclo[2.2.1]heptan-5-yl]pyrazolo[1,5-a]pyrimidine-3-amido}pyrazol-1-yl]cyclohexyl]methyl}piperidin-4-yl)oxy]prop-1-yn-1-yl}-1,3-benzodiazol-1-yl)-2,6-dioxopiperidin-1-yl]methyl}carbamoyl)methyl]carbamate (Compound 43-26, 400 mg, 66%) as a yellow solid. LCMS (ESI, m/z): 1036 [M+H]⁺.

### Step 18. Preparation of Compound 43-27

A mixture of prop-2-en-1-yl N-[({[3-(3-methyl-2-oxo-4-{3-[(1-{[(1r,4r)-4-[3-(difluoromethyl)-4-{5-[(1R,4R)-2-oxa-5-azabicyclo[2.2.1]heptan-5-yl]pyrazolo[1,5-a]pyrimidine-3-amido}pyrazol-1-yl]cyclohexyl]methyl}piperidin-4-yl)oxy]prop-1-yn-1-yl}-1,3-benzodiazol-1-yl)-2,6-dioxopiperidin-1-yl]methyl}carbamoyl)methyl]carbamate (Compound 43-26, 150 mg, 0.14 mmol, 1 equiv), Pd(PPh₃)₄ (17 mg, 0.014 mmol, 0.1 equiv) and phenylsilane (32 mg, 0.29 mmol, 2 equiv) in THE (7.5 mL) was stirred for 1 h at 0° C. under nitrogen atmosphere. LCMS indicated the reaction was completed. The solvent was removed under reduced pressure. The residue was purified by reverse flash chromatography with the following conditions: column, C18 silica gel, 80 g, 20-35 um; mobile phase, water with 0.1% FA and ACN (0% to 100% gradient in 50 min); detector, UV 254 nm. The eluent was concentrated to afford N-[3-(difluoromethyl)-1-[(1r,4r)-4-[(4-{[3-(1-{1-[(2-aminoacetamido)methyl]-2,6-dioxopiperidin-3-yl}-3-methyl-2-oxo-1,3-benzodiazol-4-yl)prop-2-yn-1-yl]oxy}piperidin-1-yl)methyl]cyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-27, 105 mg, 76%) as a white solid. LCMS (ESI, m/z): 952 [M+H]⁺.

### Step 19. Preparation of Compound (XLIII)

A mixture of (2S)-2-(2-{2-[6-(2,5-dioxopyrrol-1-yl)hexanamido]acetamido}acetamido)-3-phenylpropanoic acid (Compound 40-6, 47 mg, 0.10 mmol, 1 equiv), HATU (57 mg, 0.15 mmol, 1.5 equiv) and HOBT (14 mg, 0.10 mmol, 1 equiv) in DMF (3 mL) was stirred for 5 min at 0° C. Then N-[3-(difluoromethyl)-1-[(1r,4r)-4-[(4-{[3-(1-{1-[(2-aminoacetamido)methyl]-2,6-dioxopiperidin-3-yl}-3-methyl-2-oxo-1,3-benzodiazol-4-yl)prop-2-yn-1-yl]oxy}piperidin-1-yl)methyl]cyclohexyl]pyrazol-4-yl]-5-[(1R,4R)-2-oxa-5-azabicyclo[2.2.1]heptan-5-yl]pyrazolo[1,5-a]pyrimidine-3-carboxamide (Compound 43-27, 95 mg, 0.10 mmol, 1 equiv) was added into the above mixture followed by DIEA (26 mg, 0.20 mmol, 2 equiv). The resulting mixture was stirred for 1 h at 0° C. LCMS indicated the reaction was completed. The reaction system was purified by prep-HPLC with the following condition: Column: XSelect CSH Prep C18 OBD Column, 19*150 mm, 5 m; Mobile Phase A: Water (0.05% TFA), Mobile Phase B: ACN; Flow rate: 25 mL/min; Gradient: 24% B to 44% B in 7 min, 44% B; Wave Length: 254 nm; RT1 (min): 6.23. The collected fraction was lyophilized to afford N-[3-(difluoromethyl)-1-[(1r,4r)-4-({4-[(3-{1-[1-({2-[(2S)-2-(2-{2-[6-(2,5-dioxopyrrol-1-yl)hexanamido]acetamido}acetamido)-3-phenylpropanamido]acetamido}methyl)-2,6-dioxopiperidin-3-yl]-3-methyl-2-oxo-1,3-benzodiazol-4-yl}prop-2-yn-1-yl)oxy]piperidin-1-yl}methyl)cyclohexyl]pyrazol-4-yl]-5-[(1R,4R)-2-oxa-5-azabicyclo[2.2.1]heptan-5-yl]pyrazolo[1,5-a]pyrimidine-3-carboxamide (25.4 mg, 18%) as a white solid. LCMS (ESI, m/z): 1406 [M+H]⁺. ¹H NMR (400 MHz, DMSO-d₆) δ 9.52 (d, J=6.4 Hz, 1H), 9.00-8.80 (m, 2H), 8.41 (d, J=4.0 Hz, 1H), 8.30-8.15 (m, 3H), 8.15-7.95 (m, 3H), 7.27-7.11 (m, 9H), 7.06-6.99 (m, 2H) 6.68 (dd, J=164, 7.8 Hz, 1H), 5.50 (dd, J=13.2, 5.2 Hz, 1H), 5.31-5.09 (m, 3H), 4.78 (d, J=22.0 Hz, 1H), 4.55-4.48 (m, 3H), 4.30-4.23 (m, 1H), 3.86-3.74 (m, 5H), 3.73-3.52 (m, 10H), 3.48-3.27 (m, 4H), 3.04-2.95 (m, 6H), 2.89-2.60 (m, 3H), 2.27-2.03 (m, 8H), 2.03-1.87 (m, 5H), 1.85-1.75 (m, 2H), 1.51-1.40 (m, 4H), 1.25-1.10 (m, 4H).

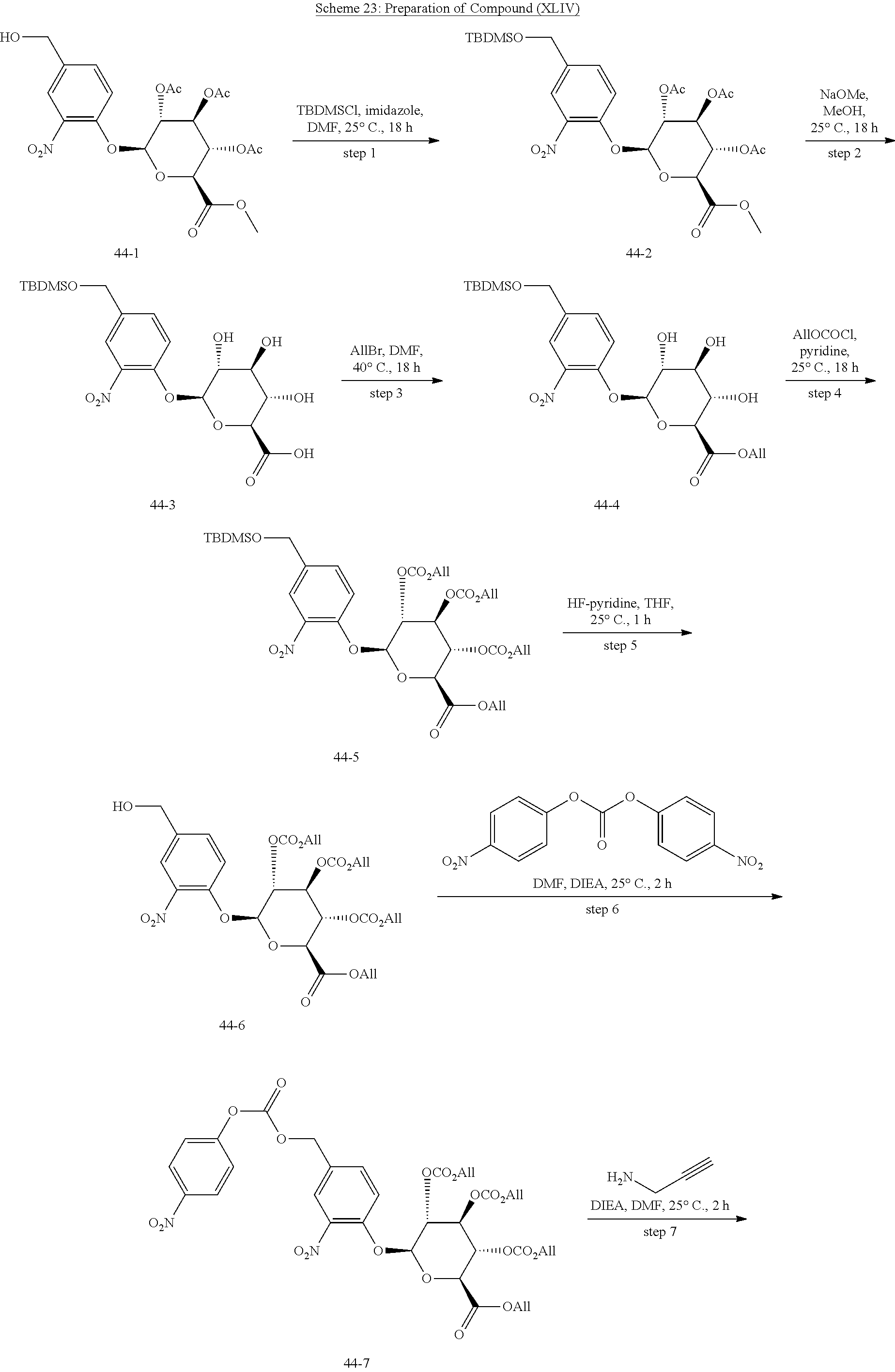

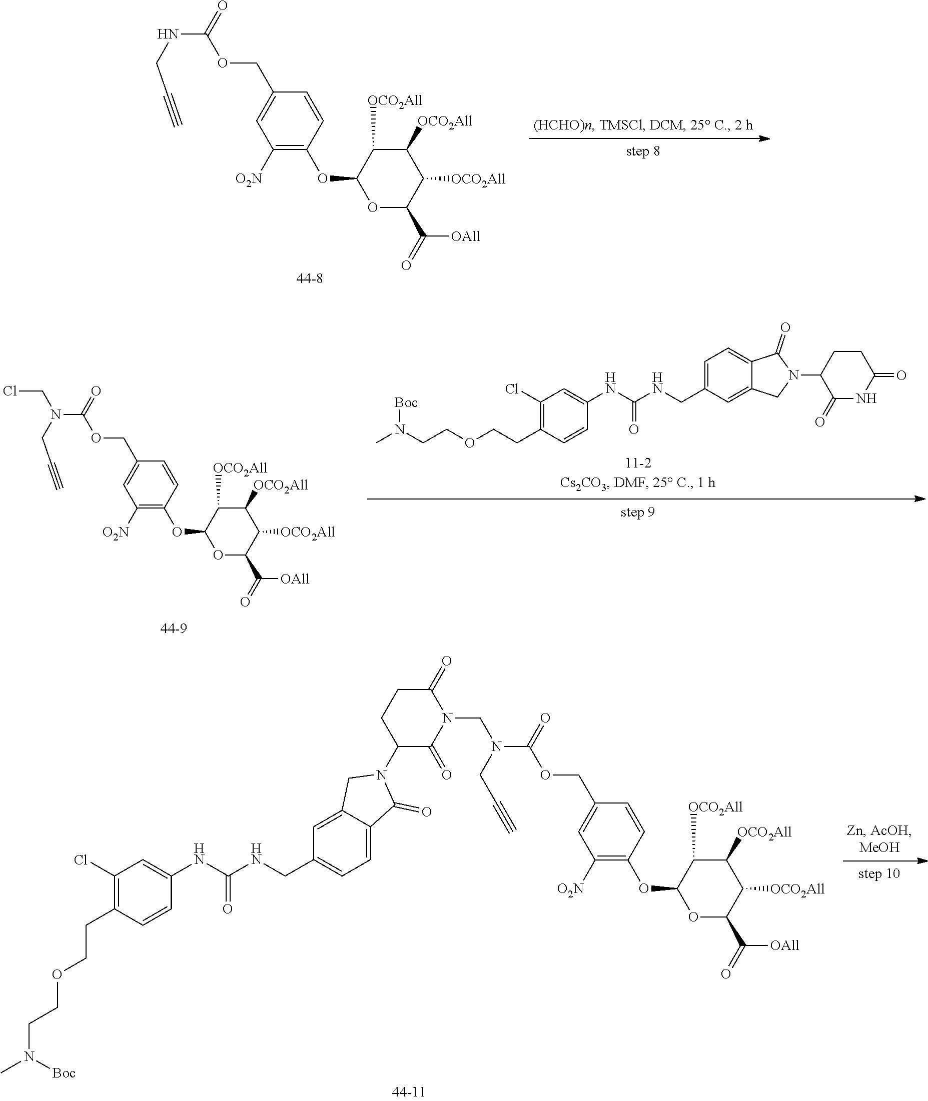

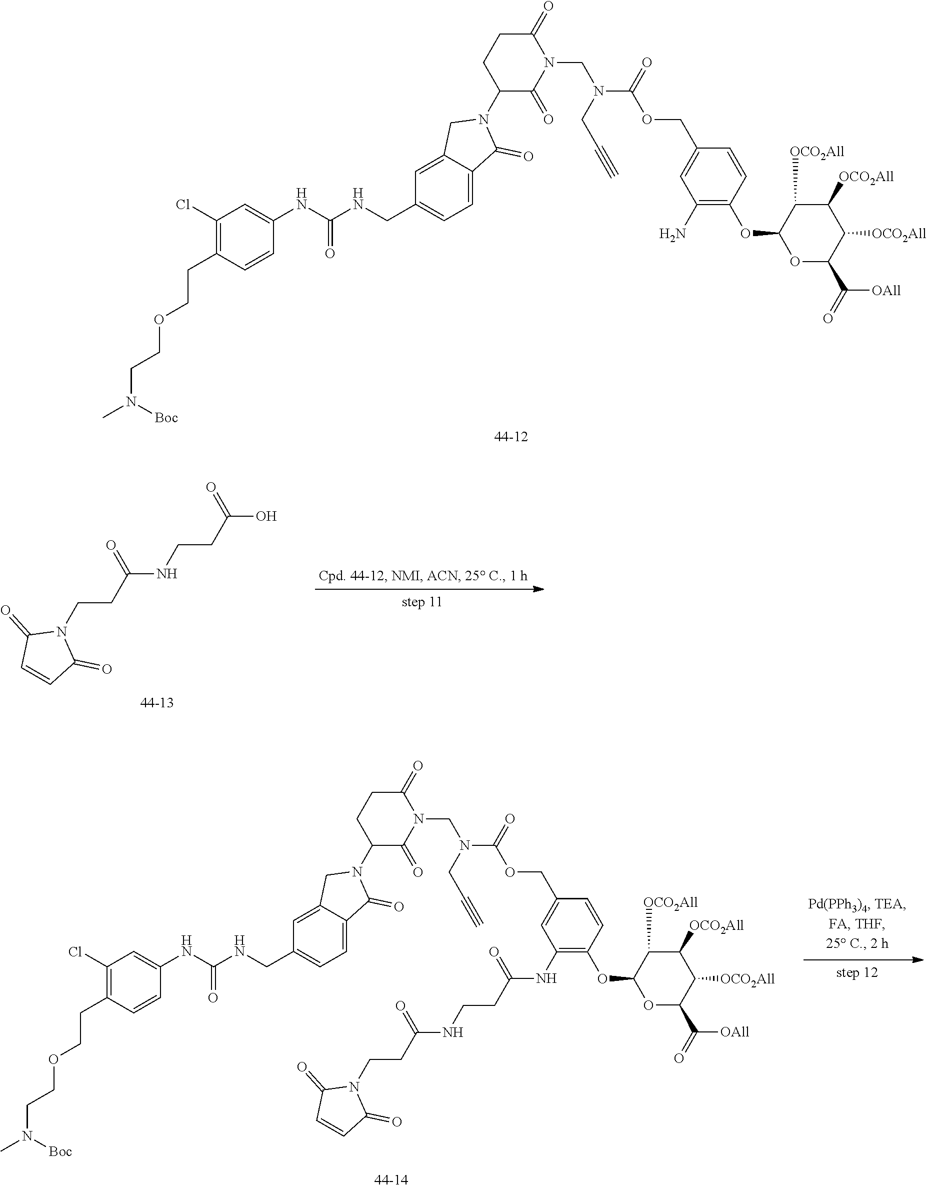

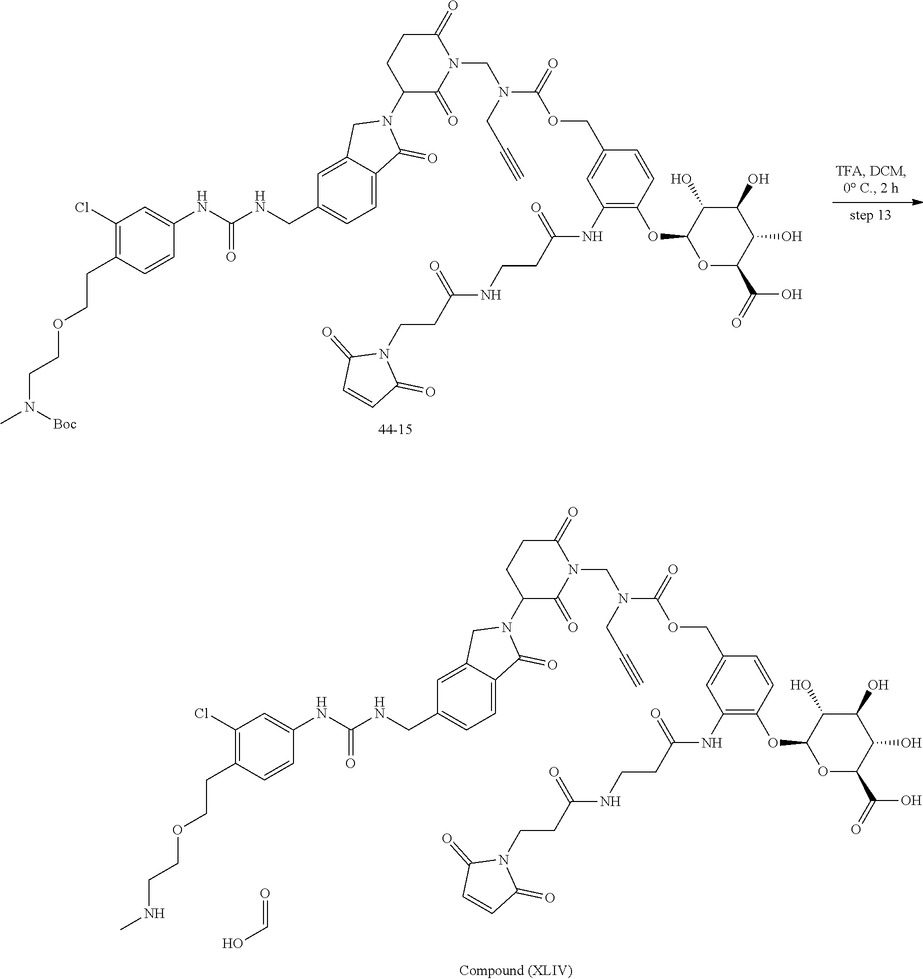 [SMILES: C#CCN(CN1C(=O)CCC(N2Cc3cc(CNC(=O)Nc4ccc(CCOCCN(C)C(=O)OC(C)(C)C)c(Cl)c4)ccc3C2=O)C1=O)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1 ; C#CCN(CN1C(=O)CCC(N2Cc3cc(CNC(=O)Nc4ccc(CCOCCNC)c(Cl)c4)ccc3C2=O)C1=O)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1 ; O=CO]

### Step 1. Preparation of Compound 44-2

To a stirred solution of methyl (2S,3S,4S,5R,6S)-3,4,5-tris(acetyloxy)-6-[4-(hydroxymethyl)-2-nitrophenoxy]oxane-2-carboxylate (Compound 44-1, 17 g, 35.02 mmol, 1 equiv) and imidazole (3.58 g, 52.53 mmol, 1.5 equiv) in N,N-dimethylformamide (170 mL) was added tert-butyldimethylsilyl chloride (7.92 g, 52.53 mmol, 1.5 equiv) dropwise at 0° C. The resulting mixture was stirred for overnight at room temperature under nitrogen atmosphere. LCMS indicated the reaction was completed. The resulting mixture was diluted with water (600 mL). The precipitated solids were collected by filtration and washed with Et₂O (3×50 mL). This resulted in methyl (2S,3S,4S,5R,6S)-3,4,5-tris(acetyloxy)-6-(4-{[(tert-butyldimethylsilyl)oxy]methyl}-2-nitrophenoxy)oxane-2-carboxylate (Compound 44-2, 16.1 g, 76%) as an off-white solid. LCMS (ESI, m/z): 617 [M+H+NH₃]⁺.

### Step 2. Preparation of Compound 44-3

To a stirred solution of methyl (2S,3S,4S,5R,6S)-3,4,5-tris(acetyloxy)-6-(4-{[(tert-butyldimethylsilyl)oxy]methyl}-2-nitrophenoxy)oxane-2-carboxylate (Compound 44-2, 16 g, 26.68 mmol, 1 equiv) in methanol (320 mL) was added sodium methoxide (8.65 g, 160.09 mmol, 6.00 equiv) at room temperature. The resulting mixture was stirred for overnight at room temperature under nitrogen atmosphere. LCMS indicated the reaction was completed. The resulting mixture was concentrated under vacuum. This resulted in (2S,3S,4S,5R,6S)-6-(4-{[(tert-butyldimethylsilyl)oxy]methyl}-2-nitrophenoxy)-3,4,5-trihydroxyoxane-2-carboxylic acid (Compound 44-3, 10 g, 81%) as an off-white solid. LCMS (ESI, m/z): 477 [M+H+NH₃]⁺; ¹H NMR (400 MHz, DMSO-d₆) δ 8.50 (s, 1H), 7.76 (d, J=2.0 Hz, 1H), 7.57-7.49 (m, 1H), 7.41 (d, J=8.8 Hz, 1H), 5.05 (d, J=7.2 Hz, 1H), 4.72 (s, 2H), 3.49 (d, J=10.2 Hz, 1H), 3.30-3.08 (m, 6H), 0.91 (s, 9H), 0.09 (s, 6H).

### Step 3. Preparation of Compound 44-4

To a stirred solution of (2S,3S,4S,5R,6S)-6-(4-{[(tert-butyldimethylsilyl)oxy]methyl}-2-nitrophenoxy)-3,4,5-trihydroxyoxane-2-carboxylic acid (Compound 44-3, 10 g, 21.76 mmol, 1.00 equiv) in N,N-dimethylformamide (100 mL) was added allyl bromide (6.58 g, 54.40 mmol, 2.50 equiv) in portions at room temperature under N₂ atmosphere. The resulting mixture was stirred for overnight at 40° C. LCMS indicated the reaction was completed. The mixture was allowed to cool down to room temperature. The resulting mixture was diluted with water (500 mL). The resulting mixture was extracted with ethyl acetate (3×200 mL). The combined organic layers were washed with brine (5×200 mL), dried over anhydrous sodium sulfate, filtered and concentrated under reduced pressure. The residue was purified by silica gel column chromatography, eluted with petroleum ether/ethyl acetate (12:1) to afford prop-2-en-1-yl (2S,3S,4S,5R,6S)-6-(4-{[(tert-butyldimethylsilyl)oxy]methyl}-2-nitrophenoxy)-3,4,5-trihydroxyoxane-2-carboxylate (Compound 44-4, 10 g, 91%) as a white solid. LCMS (ES, m/z): 517 [M+H+NH₃]⁺, 522 [M+Na]⁺

### Step 4. Preparation of Compound 44-5

To a stirred solution of prop-2-en-1-yl (2S,3S,4S,5R,6S)-6-(4-{[(tert-butyldimethylsilyl)oxy]methyl}-2-nitrophenoxy)-3,4,5-trihydroxyoxane-2-carboxylate (Compound 44-4, 10 g, 20.01 mmol, 1 equiv) in pyridine (200 mL) was added prop-2-en-1-yl carbonochloridate (72.38 g, 600.48 mmol, 30 equiv) dropwise at 0° C. The resulting mixture was stirred for overnight at room temperature under nitrogen atmosphere. LCMS indicated the reaction was completed. The resulting mixture was diluted with water (500 mL). The resulting mixture was extracted with ethyl acetate (3×200 mL). The combined organic layers were washed with brine (5×200 mL), dried over anhydrous sodium sulfate, filtered, and concentrated under reduced pressure. The residue was purified by silica gel column chromatography, eluted with petroleum ether/ethyl acetate (3:1) to afford prop-2-en-1-yl (2S,3S,4S,5R,6S)-6-(4-{[(tert-butyldimethylsilyl)oxy]methyl}-2-nitrophenoxy)-3,4,5-tris({[prop-2-en-1-yloxy]carbonyl}oxy)oxane-2-carboxylate (Compound 44-5, 11.7 g, 83%) as yellow oil. LCMS (ESI, m/z): 769 [M+H+NH₃]⁺.

### Step 5. Preparation of Compound 44-6

To a stirred solution of prop-2-en-1-yl (2S,3S,4S,5R,6S)-6-(4-{[(tert-butyldimethylsilyl)oxy]methyl}-2-nitrophenoxy)-3,4,5-tris({[prop-2-en-1-yloxy]carbonyl}oxy)oxane-2-carboxylate (Compound 44-5, 11.6 g, 15.42 mmol, 1 equiv) in tetrahydrofuran (240 mL) was added hydrogen fluoride pyridine (60 mL) dropwise at 0° C. The resulting mixture was stirred for 1 h at room temperature under nitrogen atmosphere. LCMS indicated the reaction was completed. The resulting mixture was concentrated under vacuum. The residue was purified by silica gel column chromatography, eluted with petroleum ether/ethyl acetate (1:1) to afford prop-2-en-1-yl (2S,3S,4S,5R,6S)-6-[4-(hydroxymethyl)-2-nitrophenoxy]-3,4,5-tris({[(prop-2-en-1-yloxy]carbonyl}oxy)oxane-2-carboxylate (Compound 44-6, 8.9 g, 90%) as a yellow oil. LCMS (ESI, m/z): 655 [M+H+NH₃]⁺.

### Step 6. Preparation of Compound 44-7

To a stirred solution of prop-2-en-1-yl (2S,3S,4S,5R,6S)-6-[4-(hydroxymethyl)-2-nitrophenoxy]-3,4,5-tris({[prop-2-en-1-yloxy]carbonyl}oxy)oxane-2-carboxylate (Compound 44-6, 8.8 g, 13.80 mmol, 1 equiv) and bis(4-nitrophenyl) carbonate (4.62 g, 15.18 mmol, 1.1 equiv) in N,N-dimethylformamide (190 mL) was added N,N-diisopropylethylamine (3.57 g, 27.60 mmol, 2 equiv) dropwise at room temperature The resulting mixture was stirred for 2 h at room temperature under nitrogen atmosphere. LCMS indicated the reaction was completed. The resulting mixture was diluted with water (600 mL). The resulting mixture was extracted with ethyl acetate (3×200 mL). The combined organic layers were washed with brine (3×200 mL), dried over anhydrous sodium sulfate, filtered and concentrated under reduced pressure. The residue was purified by silica gel column chromatography, eluted with petroleum ether/ethyl acetate (3:1) to afford prop-2-en-1-yl (2S,3S,4S,5R,6S)-6-(2-nitro-4-{[(4-nitrophenoxycarbonyl)oxy]methyl}phenoxy)-3,4,5-tris({[prop-2-en-1-yloxy]carbonyl}oxy)oxane-2-carboxylate (Compound 44-7, 8 g, 72%) as a yellow oil. LCMS (ESI, m/z): 802 [M+H]⁺.

### Step 7. Preparation of Compound 44-8

To a stirred solution of prop-2-en-1-yl (2S,3S,4S,5R,6S)-6-(2-nitro-4-{[(4-nitrophenoxycarbonyl)oxy]methyl}phenoxy)-3,4,5-tris({[prop-2-en-1-yloxy]carbonyl}oxy)oxane-2-carboxylate (Compound 44-6, 3.5 g, 4.36 mmol, 1 equiv) in N,N-dimethylformamide (35 mL) was added 2-propynylamine (0.48 g, 8.72 mmol, 2 equiv) at 0° C. The resulting mixture was stirred for 2 h at room temperature under nitrogen atmosphere. LCMS indicated the reaction was completed. The mixture was purified by reversed-phase flash chromatography with the following conditions: column, C18 silica gel; mobile phase, acetonitrile in water (0.05% TFA), 5% to 80% gradient in 30 min; detector, UV 254 nm. The collected fraction was lyophilized to afford prop-2-en-1-yl (2S,3S,4S,5R,6S)-6-[2-nitro-4-({[(prop-2-yn-1-yl)carbamoyl]oxy}methyl)phenoxy]-3,4,5-tris({[prop-2-en-1-yloxy]carbonyl}oxy)oxane-2-carboxylate (Compound 44-8, 1.6 g, 51%) as a yellow oil. LCMS (ESI, m/z): 736 [M+H+NH₃]⁺

### Step 8. Preparation of Compound 44-9

To a stirred solution of prop-2-en-1-yl (2S,3S,4S,5R,6S)-6-[2-nitro-4-({[(prop-2-yn-1-yl)carbamoyl]oxy}methyl)phenoxy]-3,4,5-tris({[prop-2-en-1-yloxy]carbonyl}oxy)oxane-2-carboxylate (Compound 44-8, 1.4 g, 1.94 mmol, 1 equiv) and paraformaldehyde (0.12 g, 3.89 mmol, 2 equiv) in dichloromethane (140 mL) was added trimethylsilyl chloride (0.42 g, 3.89 mmol, 2 equiv) dropwise at 0° C. The resulting mixture was stirred for 2 h at room temperature under nitrogen atmosphere. The reaction mixture was derived with methanol for LCMS test. The resulting mixture was concentrated under reduced pressure to afford prop-2-en-1-yl (2S,3S,4S,5R,6S)-6-[4-({[chloromethyl(prop-2-yn-1-yl)carbamoyl]oxy}methyl)-2-nitrophenoxy]-3,4,5-tris({[prop-2-en-1-yloxy]carbonyl}oxy)oxane-2-carboxylate (Compound 44-9). The crude product was used in the next step directly without further purification. LCMS (ESI, m/z): 763 [M+H]⁺ (derivative with MeOH).

### Step 9. Preparation of Compound 44-11

To a stirred solution of prop-2-en-1-yl (2S,3S,4S,5R,6S)-6-[4-({[chloromethyl(prop-2-yn-1-yl)carbamoyl]oxy}methyl)-2-nitrophenoxy]-3,4,5-tris({[prop-2-en-1-yloxy]carbonyl}oxy)oxane-2-carboxylate (Compound 44-9, 1.3 g, 1.69 mmol, 1 equiv) and tert-butyl N-[2-(2-{2-chloro-4-[({[2-(2,6-dioxopiperidin-3-yl)-1-oxo-3H-isoindol-5-yl]methyl}carbamoyl)amino]phenyl}ethoxy)ethyl]-N-methylcarbamate (Compound 11-2, 1.06 g, 1.69 mmol, 1 equiv) in N,N-dimethylformamide (13 mL) was added cesium carbonate (0.55 g, 1.69 mmol, 1 equiv) at room temperature. The resulting mixture was stirred for 1 h at room temperature under nitrogen atmosphere. LCMS indicated the reaction was completed. The mixture was purified by reversed-phase flash chromatography with the following conditions: column, C18 silica gel; mobile phase, acetonitrile in water (0.05% TFA), 5% to 80% gradient in 30 min; detector, UV 254 nm. The collected fraction was lyophilized to afford prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]-2-nitrophenoxy}-3,4,5-tris({[prop-2-en-1-yloxy]carbonyl}oxy)oxane-2-carboxylate (Compound 44-11, 360 mg, 15%) as a white solid. LCMS (ESI, m/z): 1358 [M+H]⁺

### Step 10. Preparation of Compound 44-12

To a stirred solution of prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]-2-nitrophenoxy}-3,4,5-tris({[prop-2-en-1-yloxy]carbonyl}oxy)oxane-2-carboxylate (Compound 44-11, 350 mg, 0.25 mmol, 1 equiv) in MeOH (3 mL) and acetic acid (3 mL) was added Zn (125 mg, 1.91 mmol, 10 equiv) at room temperature. The resulting mixture was stirred for overnight at room temperature. LCMS indicated the reaction was completed. The resulting mixture was filtered, the filter cake was washed with methanol (3×5 mL). The filtrate was concentrated under reduced pressure. The residue was purified by reversed-phase flash chromatography with the following conditions: column, C18 silica gel; mobile phase, acetonitrile in water (0.05% TFA), 5% to 80% gradient in 30 min; detector, UV 254 nm. The collected fraction was lyophilized to afford prop-2-en-1-yl (2S,3 S,4S,5R,6S)-6-{2-amino-4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]phenoxy}-3,4,5-tris({[prop-2-en-1-yloxy]carbonyl}oxy)oxane-2-carboxylate (Compound 44-12, 110 mg, 32.14%) as a white solid. LCMS (ESI, m/z): 1328 [M+H]⁺; ¹H NMR (400 MHz, DMSO-d₆) δ 8.81 (s, 1H), 7.77-7.66 (m, 2H), 7.53 (s, 1H), 7.45 (d, J=8.0 Hz, 1H), 7.19 (t, J=7.2 Hz, 2H), 6.98 (d, J=8.0 Hz, 1H), 6.84 (s, 2H), 6.70 (s, 1H), 6.57 (s, 1H), 6.26 (s, 1H), 6.09 (s, 1H), 5.90-5.88 (m, 4H), 5.47-5.17 (m, 12H), 4.93 (s, 3H), 4.81 (s, 2H), 4.79-4.55 (m, 9H), 4.42 (d, J=5.6 Hz, 3H), 3.99 (s, 2H), 3.55-3.51 (m, 5H), 3.31-3.14 (m, 4H), 2.85-2.75 (m, 6H), 1.38 (s, 9H).

### Step 11. Preparation of 44-14

To a stirred solution of Compound 44-12 (110 mg, 0.08 mmol, 1 equiv) and 3-[3-(2,5-dioxopyrrol-1-yl)propanamido]propanoic acid (Compound 44-13, 40 mg, 0.16 mmol, 2 equiv) in acetonitrile (2 mL) was added 1-methylimidazole (27 mg, 0.33 mmol, 4 equiv) at room temperature. The resulting mixture was stirred for 1 h at room temperature. LCMS indicated the reaction was completed. The resulting mixture was concentrated under vacuum. The residue was purified by reversed-phase flash chromatography with the following conditions: column, C18 silica gel; mobile phase, MeCN in Water (0.1% FA), 5% to 80% gradient in 30 min; detector, UV 254 nm. The collected fraction was lyophilized to afford prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]-2-{3-[3-(2,5-dioxopyrrol-1-yl)propanamido]propanamido}phenoxy}-3,4,5-tris({[prop-2-en-1-yloxy]carbonyl}oxy)oxane-2-carboxylate (Compound 44-14, 70 mg, 54%) as a white solid. LCMS (ESI, m/z): 1550 [M+H]⁺

### Step 12. Synthesis of Compound (44-15)

To a stirred solution of prop-2-en-1-yl (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]-2-{3-[3-(2,5-dioxopyrrol-1-yl)propanamido]propanamido}phenoxy}-3,4,5-tris({[prop-2-en-1-yloxy]carbonyl}oxy)oxane-2-carboxylate (Compound 44-14, 50 mg, 0.03 mmol, 1 equiv) and triethylamine (13 mg, 0.12 mmol, 4 equiv) in tetrahydrofuran (5 mL) was added formic acid (5 mg, 0.09 mmol, 3 equiv) and tetrakis(triphenylphosphine)palladium (15 mg, 0.012 mmol, 0.4 equiv) at room temperature under nitrogen atmosphere. The resulting mixture was stirred for 2 h at room temperature under nitrogen atmosphere. LCMS indicated the reaction was completed. The resulting mixture was concentrated under vacuum. The residue was purified by reversed-phase flash chromatography with the following conditions: column, C18 silica gel; mobile phase, MeCN in Water (0.05% TFA), 5% to 80% gradient in 30 min; detector, UV 254 nm. The collected fraction was lyophilized to afford (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]-2-{3-[3-(2,5-dioxopyrrol-1-yl)propanamido]propanamido}phenoxy}-3,4,5-trihydroxyoxane-2-carboxylic acid (Compound 44-15, 10 mg, 24%) as a white solid. LCMS (ESI, m/z): 1258 [M+H]⁺

### Step 13. Preparation of Compound (XLIV)

To a stirred solution of (2S,3S,4S,5R,6S)-6-{4-[({[(3-{5-[({[4-(2-{2-[(tert-butoxycarbonyl)(methyl)amino]ethoxy}ethyl)-3-chlorophenyl]carbamoyl}amino)methyl]-1-oxo-3H-isoindol-2-yl}-2,6-dioxopiperidin-1-yl)methyl](prop-2-yn-1-yl)carbamoyl}oxy)methyl]-2-{3-[3-(2,5-dioxopyrrol-1-yl)propanamido]propanamido}phenoxy}-3,4,5-trihydroxyoxane-2-carboxylic acid (Compound 44-15, 10 mg, 0.008 mmol, 1 equiv) in dichloromethane (1 mL) was added trifluoroacetic acid (0.2 mL) dropwise at 0° C. The resulting mixture was stirred for 2 h at 0° C. under nitrogen atmosphere. LCMS indicated the reaction was completed. The resulting mixture was concentrated under vacuum. The crude product (10 mg) was purified by Prep-HPLC with the following conditions (Column: XSelect CSH Prep C18 OBD Column, 19*150 mm, 5 m; Mobile Phase A: Water (0.1% TFA), Mobile Phase B: ACN; Flow rate: 25 mL/min; Gradient: 18% B to 38% B in 10 min, 38% B; Wave Length: 254 nm; RT1 (min): 7.75; Injection Volume: 0.7 mL; The collected fraction was lyophilized to afford (2S,3S,4S,5R,6S)-6-[4-({[({3-[5-({[(3-chloro-4-{2-[2-(methylamino)ethoxy]ethyl}phenyl)carbamoyl]amino}methyl)-1-oxo-3H-isoindol-2-yl]-2,6-dioxopiperidin-1-yl}methyl)(prop-2-yn-1-yl)carbamoyl]oxy}methyl)-2-{3-[3-(2,5-dioxopyrrol-1-yl)propanamido]propanamido}phenoxy]-3,4,5-trihydroxyoxane-2-carboxylic acid (Compound (XLIV), 1.2 mg, 13%) as a white solid. LCMS (ESI, m/z): 1158 [M+H−FA]⁺; ¹H NMR (400 MHz, CD₃OD) δ 8.20 (s, 1H), 7.78 (d, J=4.0 Hz, 1H), 7.57 (s, 2H), 7.51 (d, J=8.0 Hz, 1H), 7.23 (s, 2H), 7.22-7.13 (m, 2H), 6.72 (s, 2H), 5.58-5.35 (m, 2H), 5.26-5.20 (m, 1H), 5.12-5.11 (m, 2H), 4.54 (s, 2H), 4.48-4.22 (m, 2H), 4.17 (br s, 2H), 3.95-3.88 (m, 1H), 3.76-3.70 (m, 6H), 3.68-3.59 (m, 3H), 3.55-3.42 (m, 3H), 3.21-3.19 (m, 2H), 3.02-2.85 (m, 4H), 2.75-2.70 (m, 4H), 2.60 (s, 2H), 2.46-2.25 (m, 3H), 2.18-2.05 (m, 1H).

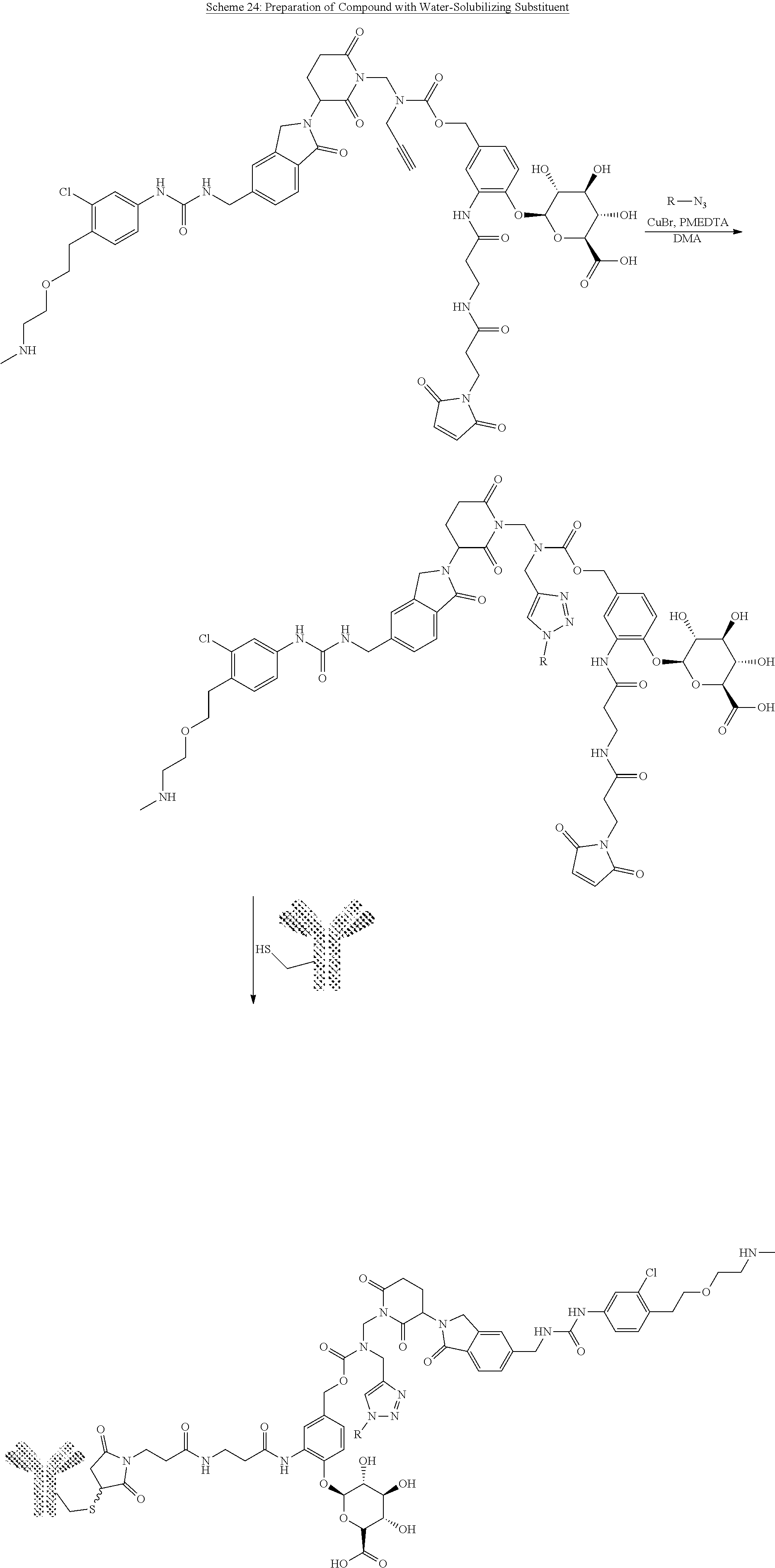

To a dry vial containing 28.75 μL degassed, anhydrous DMA are added as 20 mM stock solutions in dry degassed DMA 25 μL Compound (XLIV) (0.5 μmol, final concentration 5 mM), 5 μL CuBr (0.1 μmol), 10 μL N,N′,N″,N″-pentamethyldiethylenetriamine (0.2 μmol), and 31.25 μL (0.625 μmol) R—N₃, where R is any highly water soluble group (to aid in the conjugation of hydrophobic degraders). The reaction is stirred at ambient temperature under nitrogen and monitored by LC-MS. When Compound (XLIV) is consumed, the reaction is judged to be complete, and the crude product is used without purification for conjugation according to the procedures above.

### Example 2: General Procedure for Preparation of Antibody Conjugates

The antibody in 50 mM EPPS, 5 mM EDTA pH 7.0 buffer was treated with 12 molar equivalents of TCEP and incubated at 37° C. for 2 h to fully reduce the interchain disulfides. The reduced antibody was purified into 50 mM EPPS, 5 mM EDTA pH 7.0 by gel filtration using a Zeba 40K column. 12 molar equivalents of linker-payload was added as a stock solution in DMA such that the final concentration of DMA was 10% v/v and the resulting reaction mixture was incubated at ambient temperature for 2 h. The resulting conjugate was purified into 20 mM succinate, 8% sucrose, 0.01% Tween-20 pH 5.5 formulation buffer by gel filtration using a Zeba 40K column followed by dialysis against the formulation buffer using a Slide-a-Lyzer 10 K cassette. The purified ADC was found to have an average of 8.1 drugs/Ab by LC-MS; consist of 98.8% monomer by SEC; and contain <1.2% unconjugated linker-payload by reversed-phase HPLC. Similar procedures using other antibodies and linker-payloads give the corresponding fully-loaded ADCs.

### Example 2-1: Preparation of CD33-D Antibody—Compound (XL) Conjugate

CD33-D antibody, 8 mg/mL in 50 mM EPPS, 5 mM EDTA pH 7.0, was treated with 12 eq. TCEP and incubated at 37° C. to reduce the interchain disulfides. The reduced antibody was purified into 50 mM EPPS, 5 mM EDTA pH 7.0 by desalting using Zeba 40K desalting columns. Conjugation was effected by diluting the antibody and adding 12 eq. Compound (XL) as a stock solution in DMA such that the final reaction mixture consisted of 2 mg/mL reduced antibody+12 eq. Compound (XL) in 50 mM EPPS, 5 mM EDTA pH 7.0+20% DMA. The reaction was incubated at ambient temperature. The conjugate was purified into 20 mM succinate, 8% sucrose, 0.01% Tween-20 pH 5.5 formulation buffer using Zeba 40K desalting columns. At this point the conjugate was found to have 6.0 Compound (XL)/antibody by LC-MS.

To increase the drug loading, the pH of conjugate solution was adjusted by adding 0.1 volumes of 1M Tris pH 7.5 and an additional 5 eq. of Compound (XL) was added as a stock solution in DMA such that the final DMA concentration was 10%. After 1 h, the conjugate was purified into 20 mM succinate, 8% sucrose, 0.01% Tween-20 pH 5.5 formulation buffer by desalting using Zebra 40K desalting columns.

To remove residual free drug, 250 mg of activated charcoal was washed three times with 1 mL water, then suspended into 1 mL of the formulation buffer. 0.1 volumes of this charcoal slurry was added to the conjugate and mixed end over end for 1 h at ambient temperature. The activated charcoal was removed by filtration through a 0.22 um filter.

The conjugate was found to have 8.0 Compound (XL)/antibody by LC-MS; 87% monomer by SEC; and <1.7% free drug by mixed-mode HPLC using a HISEP column.

### Example 2-2: Preparation of CD33-D Antibody—Compound (XV) Conjugate

CD33-D antibody, 5 mg/mL in 50 mM EPPS, 5 mM EDTA was treated with 12 eq. TCEP and incubated at 37° C. for 2 h to reduce the interchain disulfides. Reduced antibody was purified into 50 mM EPPS, 5 mM EDTA pH 7.0 using Zeba 40K desalting columns.

Conjugation was affected by diluting the antibody and adding Compound (XIV) as a stock solution in DMA such that the final reaction mixture consisted of 4.0 mg/mL reduced antibody+12 eq. Compound (XIV) in 50 mM EPPS, 5 mM EDTA pH 7.0+10% DMA. The reaction was incubated for 1 h at ambient temperature. The conjugate was purified into 20 mM succinate, 8% sucrose, 0.01% Tween-20 pH 5.5 using Zebra 40K desalting columns.

Conjugate was found to have 7.9 Compound (XIV)/antibody by LC-MS; 98.9% monomer by SEC; and <0.6% free drug by mixed-mode HPLC using a HISEP column.

### Example 2-3: Preparation of Belantamab-Compound (XV) Conjugate

Belantamab, 11 mg/mL in 20 mM histidine, 250 mM sucrose, pH 6.5 was treated with 15 eq. TCEP and incubated at 37° C. for 1 h to reduce the interchain disulfides. Reduced antibody was purified into 50 mM EPPS, 5 mM EDTA pH 7.0 using Zebra 40K desalting columns.

Conjugation was affected by diluting the antibody and adding Compound (XIV) as a stock solution in DMA such that the final reaction mixture consisted of 3.0 mg/mL reduced antibody+12 eq. Compound (XIV) in 50 mM EPPS, 5 mM EDTA pH 7.0+10% DMA. The reaction was incubated for 1 h at ambient temperature, then quenched with 0.01 volumes of 100 mM N-acetylcysteine.

The conjugate was purified by two rounds of gel filtration using NAP desalting columns. In the first round, the column was equilibrated and eluted with 20 mM succinate, 8% sucrose, 0.01% Tween-20 pH 5.5+10% DMA. In the second round, the column was equilibrated and eluted with 20 mM succinate, 8% sucrose, 0.01% Tween-20 pH 5.5. Finally, the conjugate was concentrated using a 50K MWCO Amicon centrifugal concentrator.

Conjugate was found to have 8 Compound (XIV)/antibody by LC-MS; 98.4% monomer by SEC; and 2.5% free drug by mixed-mode HPLC using a HISEP column.

### Example 2-4: Preparation of HER2-B Antibody-Compound (V) Conjugate

HER2-B antibody, 4.4 mg/mL in 50 mM EPPS, 5 mM EDTA was treated with 15 eq. TCEP and incubated at 37° C. for 2 h to reduce the interchain disulfides. Reduced antibody was purified into 50 mM EPPS, 5 mM EDTA pH 7.0 using Zeba 40K desalting columns.

Conjugation was effected by diluting the antibody and adding Compound (XIV) as a stock solution in DMA such that the final reaction mixture consisted of 4.0 mg/mL reduced antibody+12 eq. Compound (XIV) in 50 mM EPPS, 5 mM EDTA pH 7.0+10% DMA cosolvent. The reaction was incubated for 1 h at ambient temperature. Then purified into 20 mM succinate, 8% sucrose, 0.01% Tween-20 pH 5.5 using Zebra 40K desalting columns.

Conjugate was found to have 7.9 Compound (XIV)/antibody by LC-MS; 98.8% monomer by SEC; and <0.6% free drug by mixed-mode HPLC using a HISEP column.

### Example 2-4: Preparation of HER2-A Antibody-Compound (XLII) Conjugate

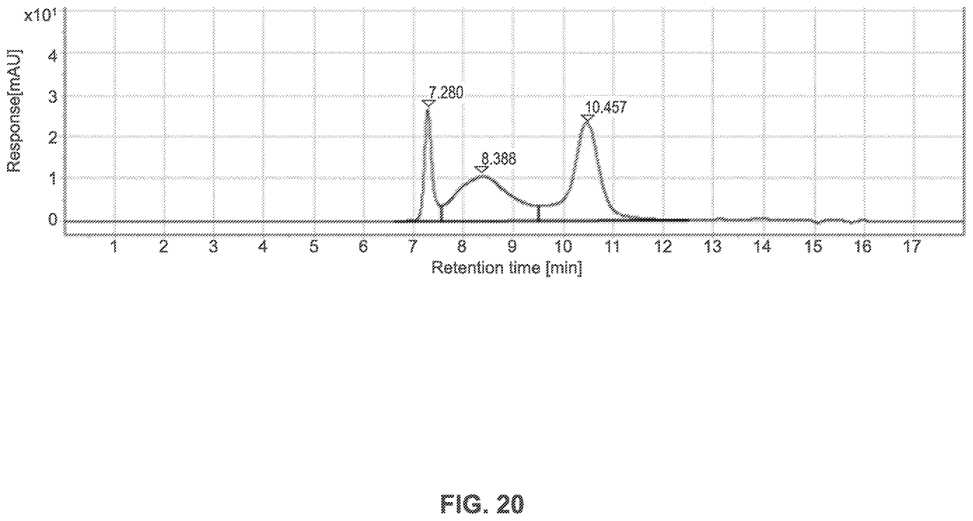

HER2-A antibody, 8 mg/mL in 50 mM EPPS, 5 mM EDTA pH 7.0, was treated with 2.25 eq. TCEP and incubated at 37° C. for 2 h to partially reduce the interchain disulfides. After cooling to ambient temperature, conjugation was effected by diluting the reduced antibody and adding Compound (XLII) as a stock solution in DMA such that the final reaction mixture consisted of 2.0 mg/mL reduced antibody+7 eq. Compound (XLII) in 50 mM EPPS, 5 mM EDTA pH 7.0+20% DMA cosolvent. The reaction was incubated for 1 h at ambient temperature. The conjugate was purified into 20 mM succinate, 8% sucrose, 0.01% Tween-20 pH 5.5 formulation buffer using Zeba 40K columns, followed by dialysis against the formulation buffer using 10K MWCO Slide-a-Lyzer cassettes. Conjugate was found to have 3.3 Compound (XLII)/Ab by LC-MS, and 48.6% monomer by SEC (see FIG. 20). Conjugation to native cysteines was found to cause significant aggregation of the antibody.

### Example 2-5: Preparation of HER2-A Antibody—Compound (XLII) Conjugate

HER2-A antibody was buffer exchanged into PBS pH 7.4 using Zeba 40K desalting columns. Antibody at 6.5 mg/mL was fully reduced by treating with 12 eq. of TCEP and incubating at 37° C. for 2 h. After cooling to ambient temperature, the reduced antibody was purified into 50 mM HEPES, 1 mM EDTA pH 7.5 using Zebra 40K desalting columns. Reduced antibody, 5 mg/mL, was treated with 20 eq. dehydroascorbic acid (added as a 50 mM stock solution in DMSO) and incubated at ambient temperature for 2 hours to re-oxidize the interchain disulfides. The pH of the reduced/re-oxidized antibody solution was adjusted to 7 with 0.015 volumes of 1M acetate pH 5.0 and conjugation was effected by adding 4 eq. Compound (XLII) as a stock solution in DMA such that the final reaction mixture consisted of 2 mg/mL Ab+4 eq. Compound (XLII) in 50 mM HEPES, 1 mM EDTA pH 7+20% DMA. The reaction was incubated overnight at ambient temperature.

Conjugate was purified into 20 mM succinate, 8% sucrose, 0.01% Tween-20 pH 5.5 using a NAP5 desalting column, diluted to 4 mL, and concentrated to ˜0.25 mL using a 50K MWCO Amicon centrifugal concentrator.

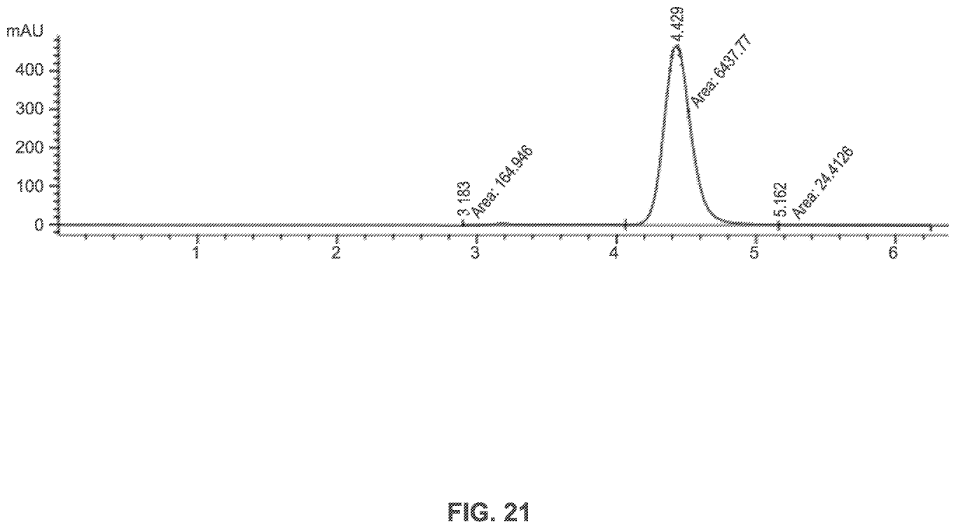

Conjugate was found to have 2.0 SMol00408/antibody by reducing RPLC-MS; 97.1% monomer by SEC (see FIG. 21); and no detectable free drug by mixed-mode HPLC using a HISEP column. Pertuzumab antibody engineered with a cysteine mutant in each heavy chain domain showed significantly improved % monomer when conjugated to mal-GGFG-ARV-471 as compared to the WT Pertuzumab conjugated stocastically through interchain cysteines. Antibody conjugates with a high monomeric state tend to have longer circulation half-life and less premature clearance than ones with high aggregation, leading to a larger preclinical therapeutic index.

### Example 2-6: Preparation of CD79b-A Antibody—Compound (XLIII) Conjugate

CD79b-A antibody, 5 mg/mL in 50 mM MES, 5 mM EDTA pH 6.0, was treated with 3.25 eq. TCEP and incubated at 37° C. for 2 h to partially reduced the interchain disulfides. After cooling to ambient temperature, conjugation was effected by diluting the reduced antibody and adding Compound (XLIII) as a stock solution in DMA such that the final reaction mixture consisted of 2.0 mg/mL reduced antibody+7 eq. Compound (XLIII) in 50 mM MES, 5 mM EDTA pH 6.0+15% DMA. The conjugate was purified by two rounds of gel filtration using NAP desalting columns. In the first round, the column was equilibrated and eluted with 20 mM succinate, 8% sucrose, 0.01% Tween-20 pH 5.5+10% DMA. In the second round, the column was equilibrated and eluted with 20 mM succinate, 8% sucrose, 0.01% Tween-20 pH 5.5. Finally, the conjugate was concentrated using a 50K MWCO Amicon centrifugal concentrator. Conjugate was found to have 4.6 Compound (XLIII)/Ab by LC-MS; 98.4% monomer; and no detectable free drug by mixed-mode HPLC using a HISEP column.

### Example 3: General Stability Studies

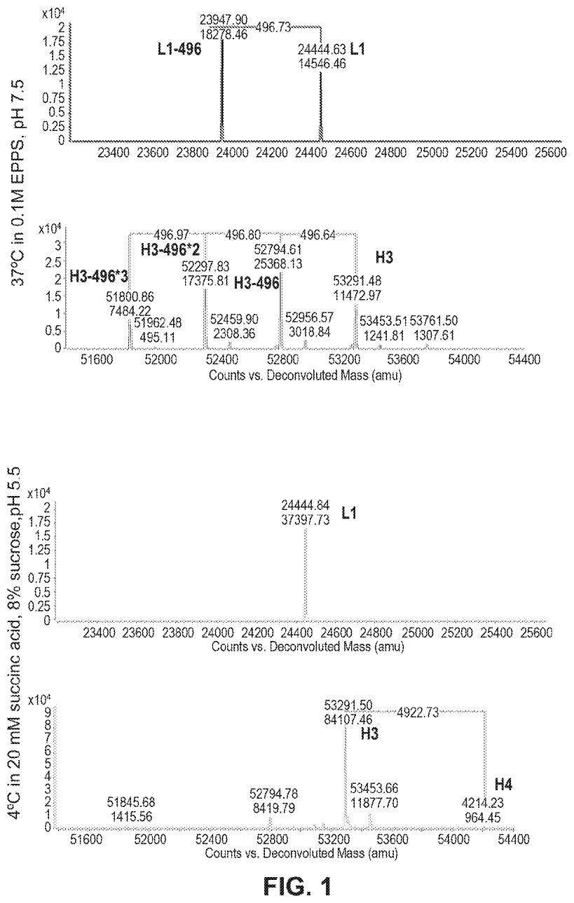

Pertuzumab-Compound (I) conjugate (DAR=8, prepared from Compound (I) according to the general procedure described above) was incubated for 24 h at 37° C. at pH7.5. As shown in the top frame of FIG. 1, LCMS analysis of the resulting product showed a significant increase in satellite peaks at −496 amu, which is indicative of carbamate cleavage and demonstrates that the compound containing this linker is not generally stable under physiological conditions. Conversely, and as shown in the bottom panel of the figure, when pertuzumab-Compound (I) conjugate was stored at 4° C. overnight at pH 5.5 (control conditions), none of the −496 amu peak was observed.

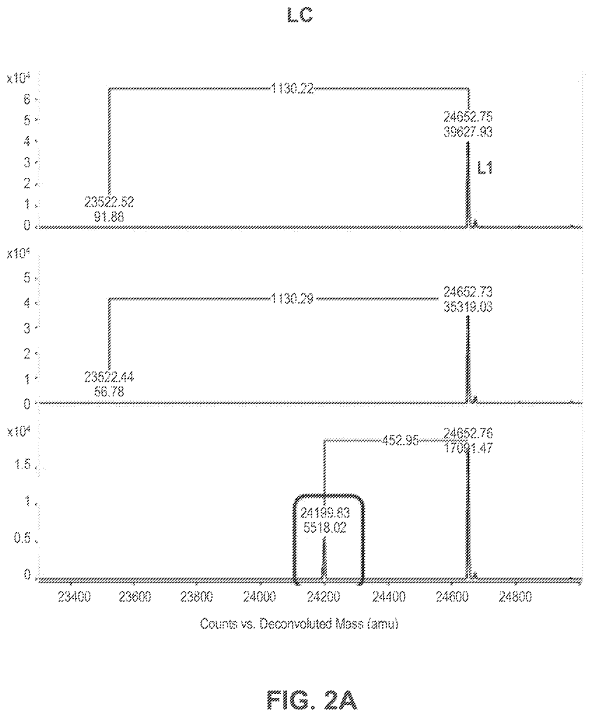

Similarly, as shown in FIGS. 2A and 2B, Pertuzumab-Compound (VIII) conjugate (DAR=8, prepared from Compound (VIII) according to the general procedure described above and reduced to light chain and heavy chain subunits) showed an increase in LCMS satellite peaks at −452 amu after 24 h at 37° C. at pH7.5, indicating ester hydrolysis occurred. The top row of each frame shows the spectra of conjugate kept at pH 5.5 and stored at 4° C., the middle row shows the spectra of conjugate at pH5.5 and incubated at 37° C. for 24 hours, and the bottom row shows the spectra of conjugate incubated at 37° C. for 24 hours at pH7.5. The presence of the −452 amu peak in the bottom row is indicative of ester hydrolysis and provides evidence that this conjugate containing the ester linker is not generally stable under physiological conditions.

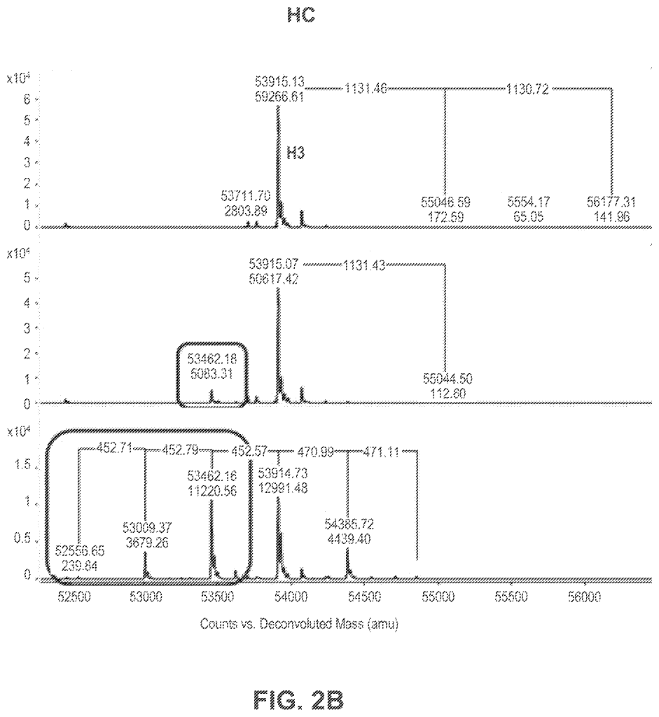

Conversely, the Pertuzumab-Compound (X) conjugate remained stable after incubation under the same conditions. As shown in FIG. 3, conjugates reduced to light chain and heavy chain subunits and incubated at 37° C. for 24 hours at pH7.5. No evidence of linker cleavage was seen by LC-MS, showing the conjugate is stable under physiological conditions.

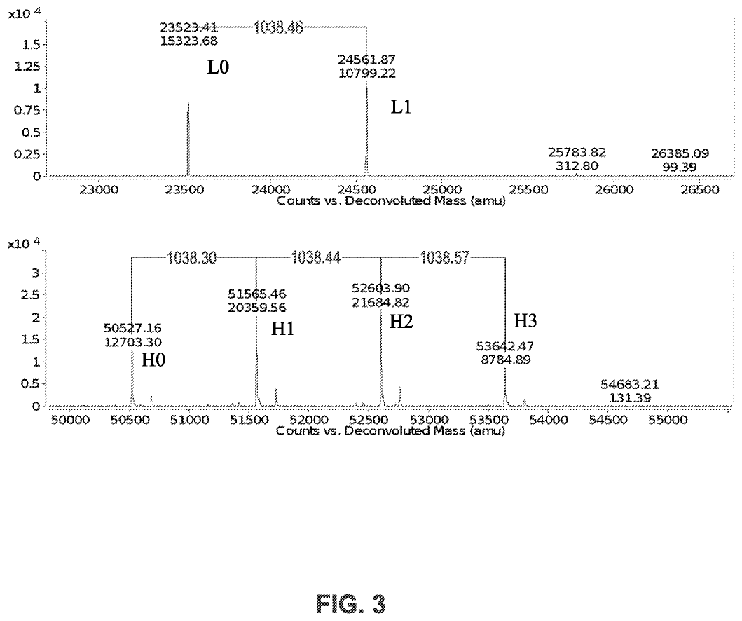

Similarly, the Pertuzumab-Compound (XI) conjugate remained stable after incubation under the same conditions. As shown in FIG. 4, only the expected species (light chain, light chain+Compound (XI); heavy chain, heavy chain+Compound (XI), heavy chain+2 Compound (XI), and heavy chain+3 Compound (XI) were observed. No peaks with masses consistent with light chain+linker fragment or heavy chain+linker fragmen, which would be indicative of linker cleavage, were observed.

### Example 4: Enzymatic Cleavage of Traceless Linker

### Papain Digestion 1

Pertuzumab-Compound (XI) conjugate was digested by papain according to the ratio of enzyme to conjugate as 1:16 at room temperature for 3 hours. The released payload was extracted with ice-cold acetonitrile, dried down and reconstituted in 95:5:0.1 H₂O: Acetonitrile: Formic acid and subsequently submitted for LC-MS analysis.

### LC-MS Analysis:

Waters SQD2 mass spectrometer equipped with Waters ACQUITY UPLC was utilized with the following settings: positive detection mode, full survey scan and SIR scan (selected ion monitoring scan targeting to m/z of 528.3 for molecular ion of neoDegrader P1). The UPLC gradient is as follows with the flowrate of 0.8 mL/min. The eluant from UPLC was splitted into UV and mass spectrometer respectively. The injection volume is 10 uL each. Mobile phase A is HPLC grade water with 0.1% trifluoracetic acid and mobile phase B is acetonitrile with 0.1% trifluoracetic acid.

| Time (min) | 0.0 | 0.5 | 2.2 | 2.6 | 2.61 | 3.0 |
|---|---|---|---|---|---|---|
| % A | 95.0 | 95.0 | 5.0 | 5 | 95.0 | 95.0 |
| % B | 5.0 | 5.0 | 95.0 | 95 | 5.0 | 5.0 |

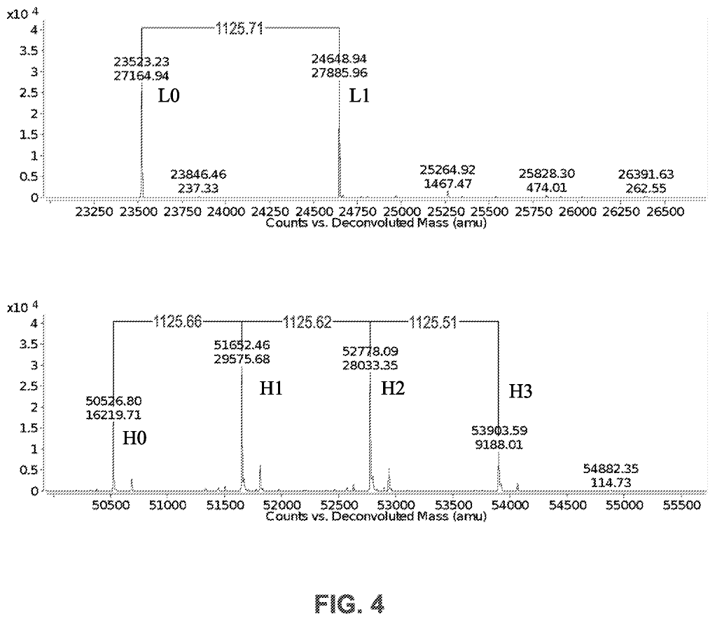

FIG. 5 shows that digestion with papain provides a peak consistent with the formation of neoDegrader P1, the structure of which is shown below. Chromatograms are also shown for control sample of Pertuzumab-Compound (XI) with no papain treatment and neoDegrader P1 standard (214 nm trace). In addition, FIG. 5 shows MS spectra for neoDegrader P1 standard and neoDegrader P1 in the papain treated conjugate sample.

Scheme 6 shows the proposed mechanism for formation of neoDegrader.

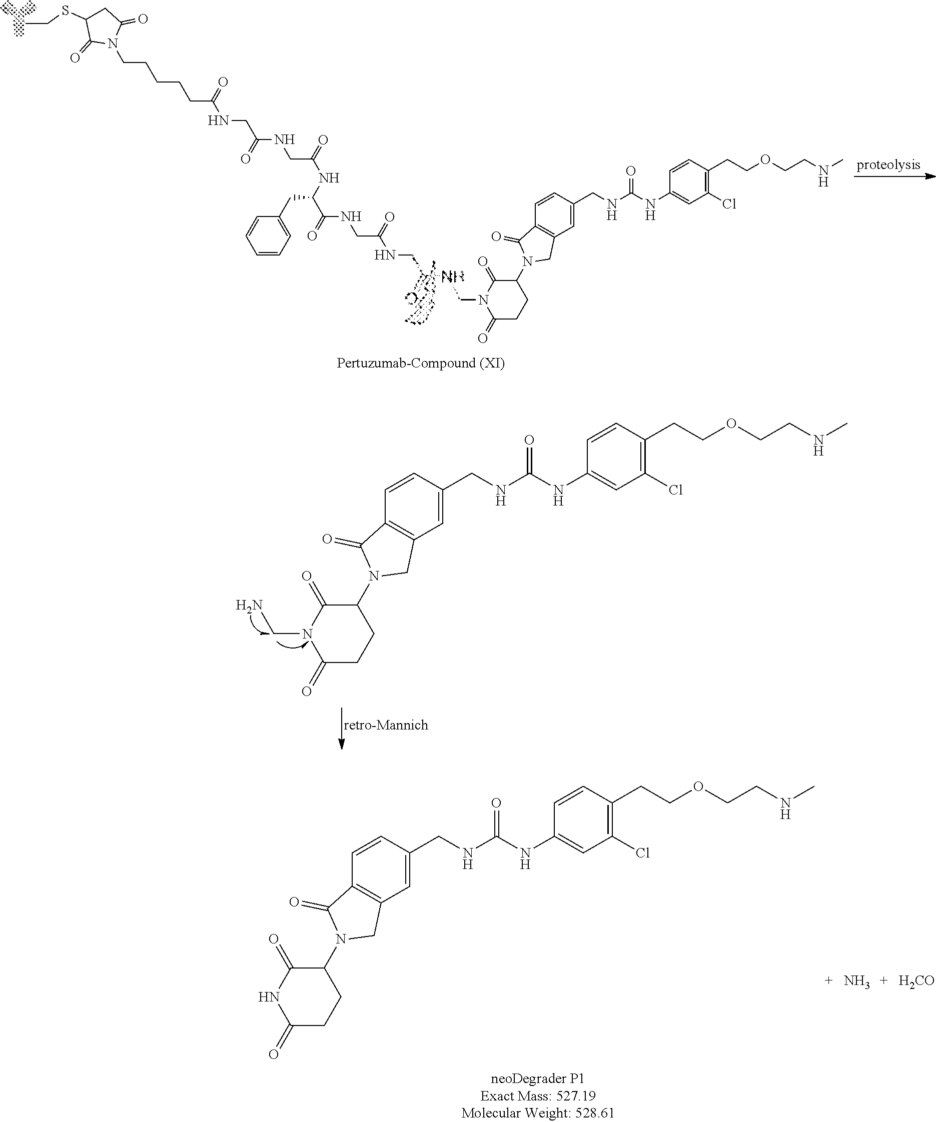

As shown in the above Examples, only the claimed traceless linker provided sufficient stability and the desired neoDegrader (Compound 18-6) udder cleavage conditions.

### Papain Digestion 2

10 μM of either Compound (XL), Compound (XI), Compound (XIV), Compound (XV), Compound (XII), or Compound (XVII) (PBS pH 7.4, 5% DMA co-solvent) was incubated with papain (2 molar equivalents) at room temperature for 2 hours with gentle shaking. The digests were extracted with 5 volumes of ice-cold acetonitrile and dried down, followed by reconstitution in 95:5:0.1 water:acetonitrile:formic acid. The samples were submitted for LC-MS analysis (Thermo Exploris 240) and using the appropriate reference standards, the released payload and/or incompletely cleaved linker byproducts were identified and quantified. Results are shown in Table 1 below.

TABLE 1
In Vitro Payload Release

| | | | % Payload | |
|---|---|---|---|---|
| Compound | Linker | Payload Released | Released | By-Products |
| XL | GGFG | Compound 40-3 | 90.2% | — |
| XVIII | GGFG | Compound 18-6 | 97.8% | — |
| XIV | GGFGG | Compound XIII | 11.6% | GGFG*Gª |
| | | | | 40.5%, |
| | | | | uncleaved 47.9% |
| XV | GGFGG | Compound 15-3 | 5.8% | GGFG*Gª |
| | | | | 78.3%, |
| | | | | uncleaved 15.8% |
| XII | GGFGG | Compound XII-1b | 9.4% | GGFG*Gª |
| | | | | 57.0%, |
| | | | | uncleaved 33.6% |
| XVII | GGFGG | Compound 9-6 | 10.7% | GGFG*Gª |
| | | | | 72.5%, |
| | | | | uncleaved 16.8% |

^{a}Byproduct is payload-CH₂NHC(O)CH₂NH₂, formed by cleavage between two glycines of linker.
^{b}Compound XII-1 structure:

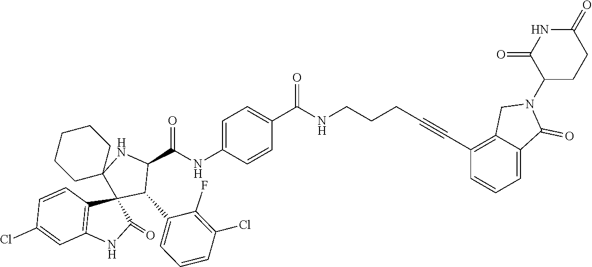 [SMILES: O=C1CCC(N2Cc3c(C#CCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1]

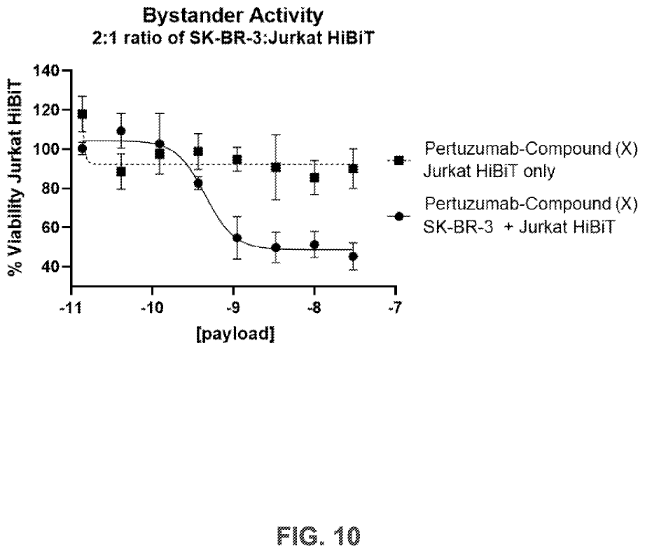

As shown in Table 1, treatment of Compounds (XL) and (XVIII) with papain released the desired payloads in excellent yield. Compounds (XIV), (XV), (XII), and (XVII), which all contain the GGFGG linker, provided lower yields of the desired payload. Without being bound by a particular theory, it is believed that papain is ineffective at efficiently cleaving this linker. This theory is supported by the results shown in FIG. 11, as the pertuzumab conjugate of Compound (XVIII), which contains a GGFG linker, and the pertuzumab conjugate of Compound (XI), which contains a GGFGG linker, both showed excellent activity against BT-474 cancer cells, indicating effective in vivo cleavage of both linkers under physiological conditions.

### β-Glucuronidase Digestion

10 μM Compound (XLI) was incubated with β-glucuronidase (Sigma, Type IX-A; 1 mg/mL solution in PBS, 2 U per ng) and the reaction mixture was incubated at 37° C. for 3 hours with gentle shaking. The digests were extracted with 5 volumes of ice-cold acetonitrile and dried down, followed by reconstitution in 95:5:0.1 water:acetonitrile:formic acid. The samples were submitted for LC-MS analysis (Thermo Exploris 240) and using the appropriate reference standards, the released payload and/or incompletely cleaved linker byproducts were identified and quantified. Results are shown in Table 2.

TABLE 2
In Vitro Payload Release

| | | Payload | % Payload | By- |
|---|---|---|---|---|
| Compound | Linker | Released | Released | Products |
| XLI | β-glucuronide | Compound 18-6 | 90.9% | — |

As shown in Table 2, treating Compound (XLI) with β-glucuronidase released the desired payload in excellent yield.

### Cysteine Digestion

1 mM Compound (XIX) was incubated with 10 molar equivalents of L-cysteine (PBS pH 7.4+20% DMA) at 37° C. for 24 hours with gentle shaking. The digests were extracted with 5× ice-cold acetonitrile and dried down, followed by reconstitution in 95:5:0.1 water:acetonitrile:formic acid. The samples were submitted for LC-MS analysis (Waters, SQD2) and using the appropriate reference standards, the released payload and/or incompletely cleaved linker byproducts were identified and quantified. Results are shown in Table 3.

TABLE 3
In vitro Payload Release

| | | Payload | % Payload | |
|---|---|---|---|---|
| Compound | Linker | Released | Released | By-Products |
| XIX | Nitrobenzenesulfonamide | Compound | sole product | — |
| | | 18-6 | identified | |

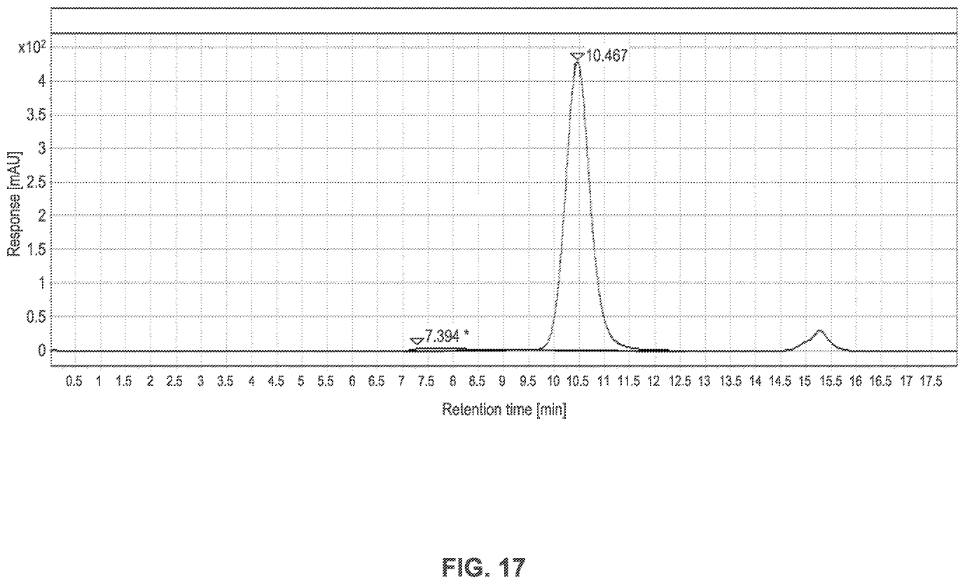

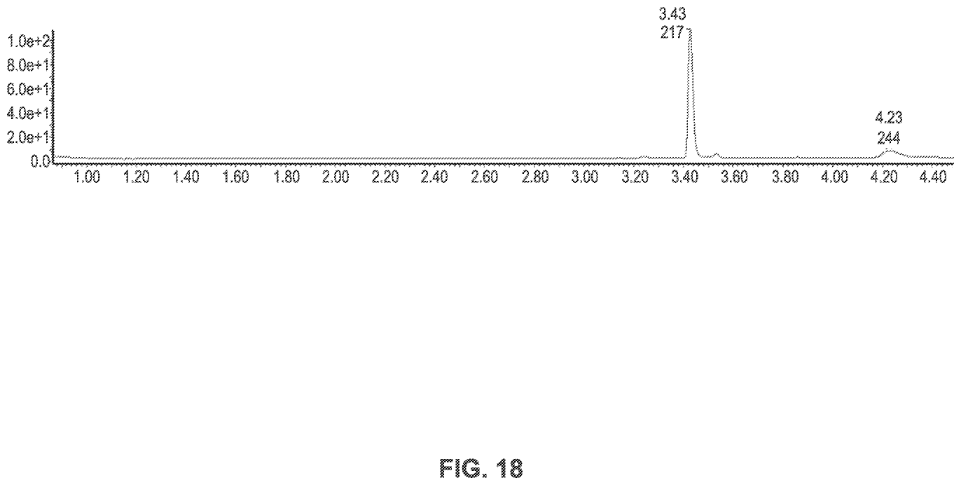

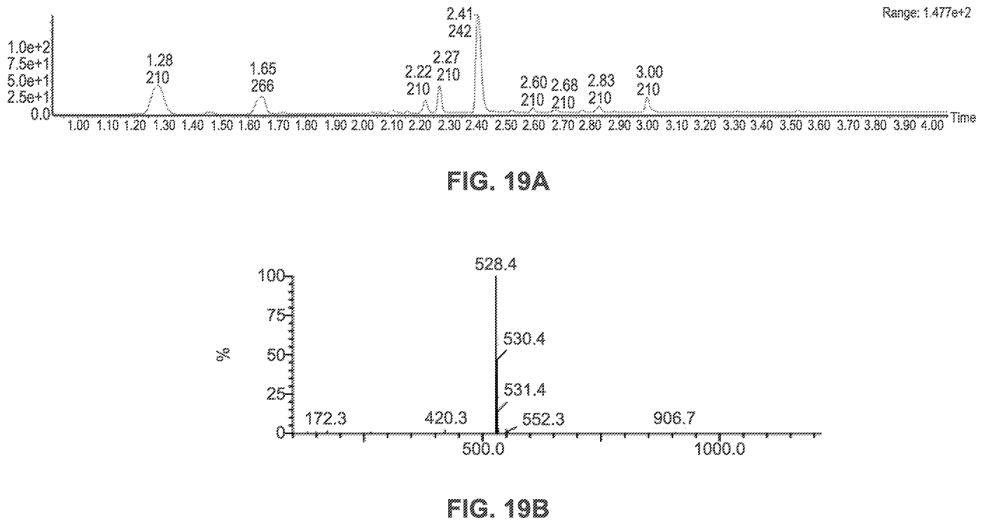

As shown in Table 3, treatment of Compound (XIX) with cysteine released the desired payload in excellent yield. Spectroscopic evidence of the release of Compound 18-6 is shown in FIGS. 18, 19A and 19B. FIG. 18 depicts the HPLC chromatogram of parent Compound (XIX) with retention time of 3.4 minutes. FIG. 19A depicts the HPLC chromatogram of the reaction mixture when Compound (XIX) is treated with cysteine. Compound (XIX) was completely consumed, and the sole identified product had a retention time of 2.41 minutes. FIG. 19B depicts the mass spectrum of the peak at retention time 2.4 minutes, m/z of 528.4, which corresponds to Compound 18-6.

### Example 5A: General Procedure for In Vitro Antiproliferation Assay

The ability of the claimed conjugates to inhibit cell growth was measured using in vitro anti-proliferation assay. Target cells were plated at 4,000-5,000 cells per well in 100 μL of complete cell growth medium (RPMI 1640, 10% fetal bovine serum and 1% Penicillin-streptomycin). Conjugates were diluted in complete cell growth medium using 3-fold serial dilutions and 100 μL was added per well. The final concentration typically ranged from 1×10⁻⁹ M to 1.52×10⁻¹³ M or 1×10⁻⁶ M to 1.53×10⁻¹¹ M. Cells were incubated at 37° C. in a humidified 5% CO₂ incubator for 5 days. Viability of remaining cells was determined by colorimetric WST-8 assay (Dojindo Molecular Technologies, Inc., Rockville, MD, US). WST-8 was added to 10% of the final volume and plates were incubated at 37° C. in a humidified 5% CO₂ incubator for 2-4 hours. Plates were analyzed by measuring the absorbance at 450 nm (A450) in a multi-well plate reader. Background A450 absorbance of wells with media and WST-8 only was subtracted from all values. The percent viability was calculated by dividing each treated sample value by the average value of wells with untreated cells. The percent viability value was plotted against the test sample concentration in a semi-log plot for each treatment. IC50 values were calculated automatically.

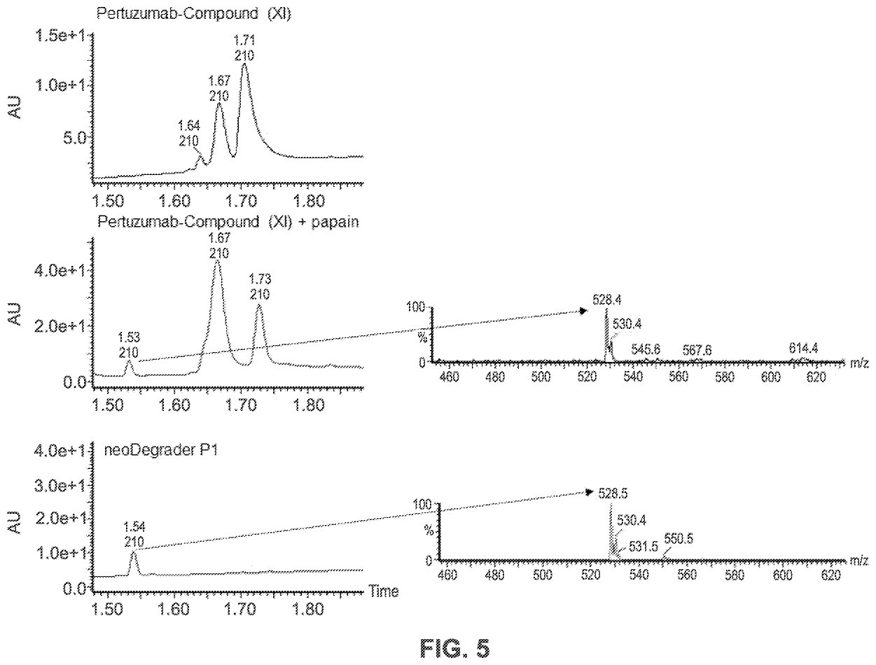

The anti-proliferative activity of rituximab and pertuzumab conjugates of Compound (X) against the BT-474 breast cancer cell line is shown in FIG. 6. As shown in the figure, non cell-binding control conjugate Rituximab-Compound (X) was found to be significantly less active against BT-474 cells.

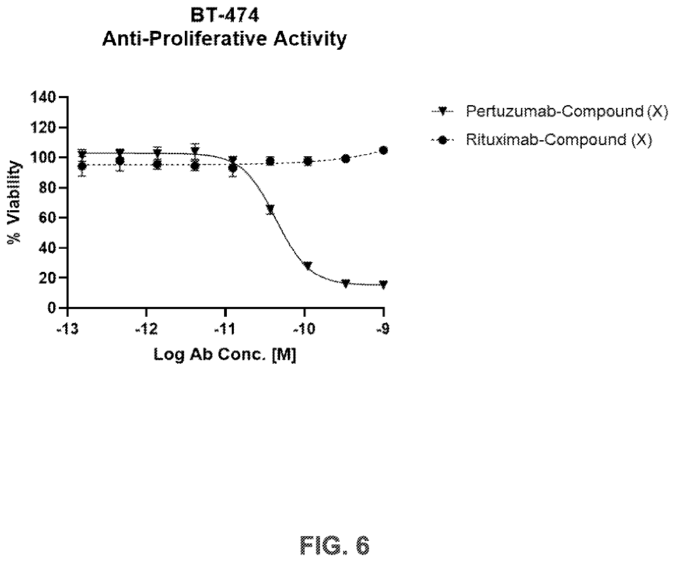

The anti-proliferative activity of rituximab and pertuzumab conjugates of Compound (X) against the NCI-N₈₇ gastric cancer cell line is shown in FIG. 7. As shown in the figure non cell-binding control conjugate Rituximab-Compound (X) was found to be significantly less active against NCI-N₈₇ cells.

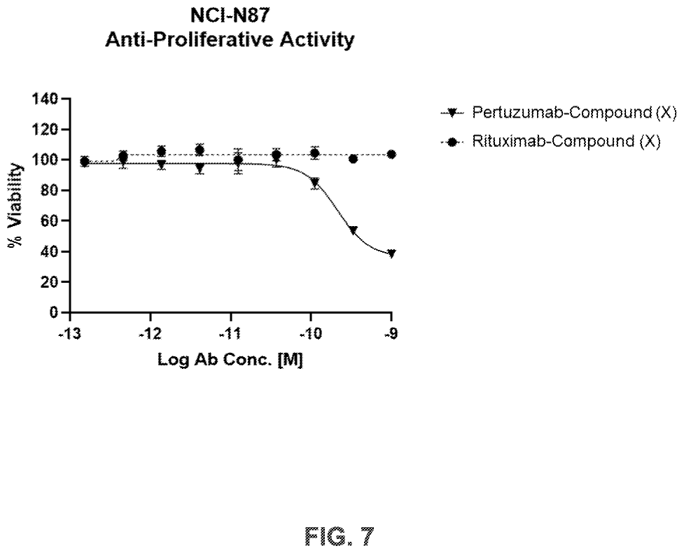

The anti-proliferative activity of rituximab and pertuzumab conjugates of Compound (XI) against the BT-474 breast cancer cell line is shown in FIG. 8. As shown in the figure, non cell-binding control conjugate Rituximab-Compound (X) was found to be significantly less active against BT-474 cells.

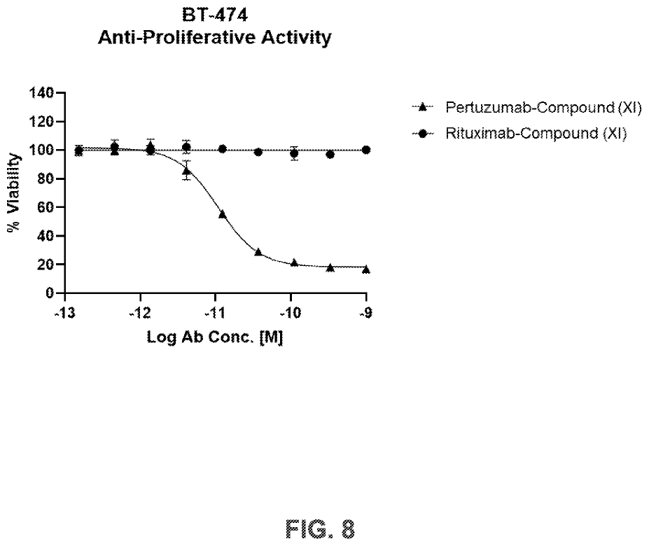

The anti-proliferative activity of rituximab and pertuzumab conjugates of Compound (XI) against the NCI-N₈₇ gastric cancer cell line is shown in FIG. 9. As shown in the figure non cell-binding control conjugate Rituximab-Compound (X) was found to be significantly less active against NCI-N₈₇ cells.

The anti-proliferative activity of pertuzumab conjugates of Compound (XVIII) and Compound (XI) against the BT-474 breast cancer cell line is shown in FIG. 11. As shown in the figure non cell-binding control conjugate Rituximab-Compound (XVIII) was found to be significantly less active against BT-474 cells.

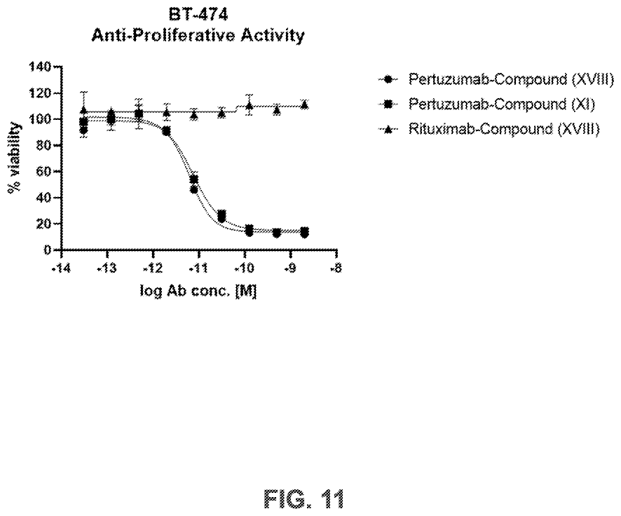

The anti-proliferative activity of the pertuzumab conjugates of Compound (XLI), Compound (XIX), and Compounds (Ie) and (Ii) as described in WO2021/198965 against the BT-474 breast cancer cell line is shown in FIG. 12. As shown in the Figure, and the corresponding table below, degraders linked to pertuzumab through the glutarimide ring have similar activity against BT-474 cells as degraders linked through the substitution on the terminal phenyl ring. In contrast, the non cell-binding control conjugates or rituximab were found to be significantly less active against BT-474 cells. This demonstrates that including a spacer capable of undergoing the retro-Mannich reaction described herein is a suitable general solution to linking and releasing a gluturamide or dihydrouracil containing degrader to an antibody or other cell binding agent. By using this technology, no additional chemical handle needs to be introduced to effect antibody based delivery to cancer cells.

TABLE 4
IC50 Values of Conjugates against BT-474 Cell Line

| Compound/Conjugate | IC50 |
|---|---|
| Pertuzumab-Compound (XLI) | 2.094E−12 |
| Pertuzumab-Compound (Ie) | 1.723E−12 |
| Pertuzumab-Compound (XIX) | 2.051E−11 |
| Pertuzumab-Compound (Ii) | 4.259E−12 |
| Pertuzumab-Compound (Ia) | 4.194E−12 |
| Rituximab-Compound (XLI) | >2E−09 |
| Rituximab-Compound (XIX) | >2E−09 |

### Example 5B: Procedure for In Vitro MV-4-11 Antiproliferation Assay

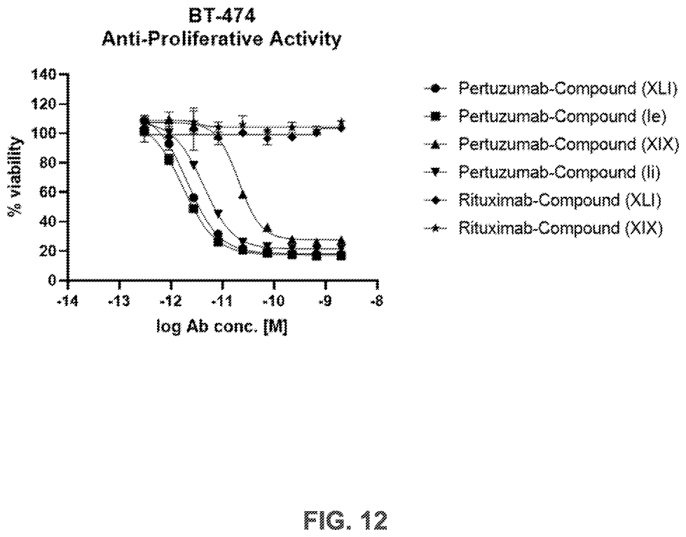

The ability of the claimed conjugates to inhibit cell growth of MV-4-11 cells was measured using an in vitro anti-proliferation assay. MV-4-11 cells were exposed to varying concentrations of conjugates or antibodies for 4 days with/without 1 μM of the blocking antibody Gemtuzumab or Trastuzumab. Viability of the cells was determined by alamarBlue. The antiproliferative activity of the gemtuzumab conjugate of Compound (XL) and the unconjuaged BRD4 degrader small molecule Compound 40-3 are showin in FIG. 13. As shown in the Figure, the gemtuzumab conjugate of Compound (XL) was highly active alone, but ˜1000 less active when CD33 surface antigens were presaturated with gemtuzumab. Preblocking with trastuzumab (antiHER2 antibody, no HER2 expression in MV-4-11) had no effect on conjugate activity. BRD4 degrader small molecule Compound 40-3 was also highly active. The antibodies alone were not active up to 1 mM.

### Example 5C: Procedure for In Vitro BRD4 Protein Degradation Assay

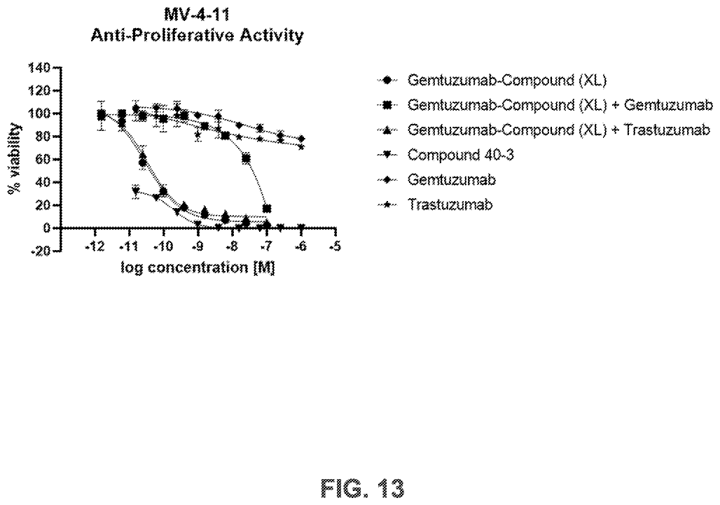

The ability of the claimed conjugates to degrade BRD4 was measured using an in vitro degradation assay. MV4-11 cells were incubated with test article overnight. Protein was loaded to 4-12% NuPAGE Bis-Tris gel. Western blot with rabbit anti-BRD4 Ab (Cell Signaling #13440) was performed at 1:1,000 dilution and re-blotting with β-actin HRP (Cell Signaling #5125) was performed at 1:1,000. The degradation activity of the gemtuzumab conjugate Compound (XL) and the unconjuaged BRD4 degrader small molecule Compound 40-3 are shown in FIG. 14. Compound 40-3 caused a >90% reduction in BRD4 protein band intensity at 10 nM. the gemtuzumab conjugate of Compound (XL) caused a >70% reduction in BRD4 protein band intensity at 10 nM, and BRD4 depletion was dependent on conjugate concentration.

### Example 5: In Vitro Assay to Determine Binding of Conjugate to Representative Cancer Cell Lines

Using the procedure described in Example 10, representative IC50 values were calculated showing the binding of conjugates and unconjugated antibodies to representative cancer cell lines. Results are shown in Table 5.

TABLE 5
IC50 Values of Conjugates against BT-474 Cell Line

| Conjugate | Antigen | Cell Line | EC50 Parent Ab | EC50 Conjugate |
|---|---|---|---|---|
| CD33-D Antibody- | CD33 | MV4-11 | 2.4 × 10⁻¹⁰ M | 2.7 × 10⁻¹⁰ M |
| Compound (XL) | | | | |
| CD33-D Antibody- | CD33 | MV4-11 | 2.2 × 10⁻¹⁰ M | 2.2 × 10⁻¹⁰ M |
| Compound (XIV) | | | | |
| HER2-B Antibody- | Her2 | BT-474 | 2.4 × 10⁻⁹ M | 2.0 × 10⁻⁹ M |
| Compound (XIV) | | | | |
| (Trastuzumab- | | | | |
| Compound (XIV)) | | | | |
| PSMA-D Antibody- | PSMA | LNCap | 4.8 × 10⁻¹⁰ M | 2.4 × 10⁻¹⁰ M |
| Compound (XV) | | | | |
| (J591-S239C- | | | | |
| Compound (XV)) | | | | |
| HER2-A antibody- | Her2 | Lenti-Her2 | 1.7 × 10⁻⁹ M | 8.4 × 10⁻¹⁰ M |
| Compound (XLII) | | Transfected | | |
| (Pertuzumab-S239C- | | MCF-7 | | |
| Compound (XLII)) | | | | |
| CD79b-A antibody- | CD79b | Ramos | 7.4 × 10⁻¹⁰ M | 3.0 × 10⁻¹⁰ M |
| Compound (XLIII) | | | | |
| (Polatuzumab- | | | | |
| Compound (XLIII)) | | | | |

As shown in the table, conjugation to the linker-payload had no significant effect on the antibody's target cell binding affinity.

### Example 6: General Procedure for Bystander Cell Killing Assay

The ability of drug from conjugate released from target cells to inhibit non-targeting cells (Jurkat HiBiT) growth was measured using bystander activity assay. Target cells at 2,000 cells per well and Jurkat HiBiT at 1,000 cells per well were plated in 100 μL of complete cell growth medium (RPMI 1640, 10% fetal bovine serum and 1% Penicillin-streptomycin). Conjugates were diluted in complete cell growth medium using 3-fold serial dilutions and 100 μL was added per well. The final concentration ranged from 3×10-8 M to 1.37×10⁻¹¹ M. Cells were incubated at 37° C. in a humidified 5% CO₂ incubator for 5 days. Viability of remaining Jurkat HiBiT cells was determined by NanoGlo HiBiT lytic reagent (Promega N3030). NanoGlo HiBiT lytic reagent 80 μL was added to the plates and incubated for 20 minutes at RT. Plates were analyzed by measuring the luminescence in a multi-well plate reader. Background luminescence of wells with media and NanoGlo HiBiT lytic reagent only was subtracted from all values. The percent viability was calculated by dividing each treated sample value by the average value of wells with untreated cells. The percent viability value was plotted against the test sample concentration in a semi-log plot for each treatment. IC50 values were calculated automatically.

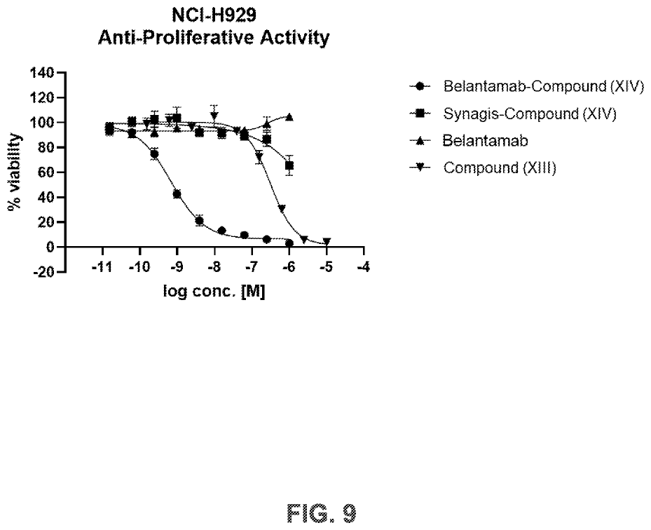

The anti-proliferative activity of pertuzumab conjugates of Compound (X) against the SK-BR-3 breast cancer cell line in the presence and absence of Jurkat HiBiT is shown in FIG. 10. As shown in the figure, the conjugate demonstrates bystander cell killing by reducing the viability of Jurkat HiBiT labeled cells (HER 2−) only in the presence of SK—BR3 (Her 2+) cells.

### Example 7: Determination of Activity of Conjugates Against In Vivo Breast Cancer Tumor Models

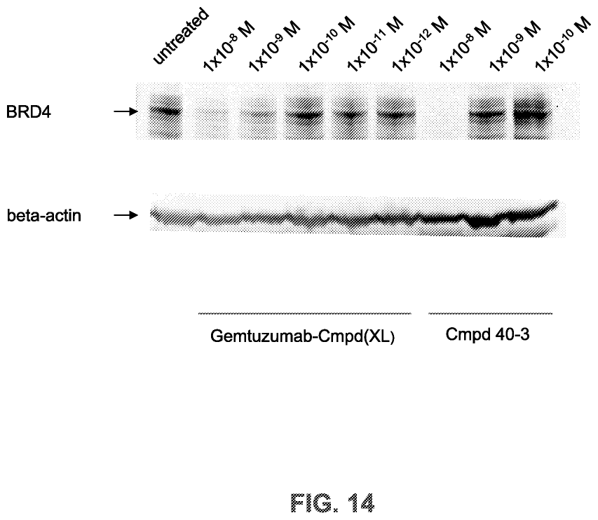

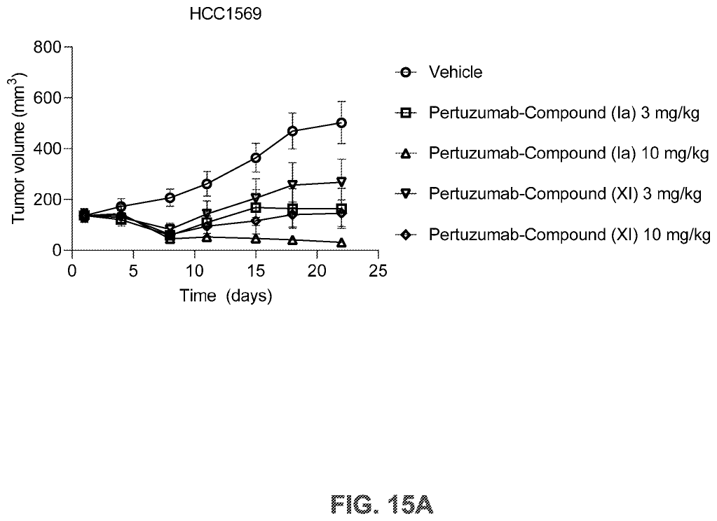

Five- to eight-week old female ICR SCID mice were sourced from Taconic. Mice were orthotopically implanted with 1e7 HCC1569 human breast cancer cells suspended in a mixture of Matrigel and culture media in the inguinal fat pad. Tumors were monitored for growth several times per week using calipers, and mice were randomly allocated into treatment groups to achieve approximately 100-150 mm3 starting tumor volumes. Following group allocation, mice were treated with a single 10 ml/kg lateral tail vein injection of either a Pertuzumab-Compound (XI) conjugate or a Pertuzumab-Compound (Ia) conjugate as described in WO2021/198965 at either 3 mg/kg or 10 mg/kg. After injection, mice were monitored daily and measured twice weekly for body weight and tumor volume. Tumor volume was determined using the standard calculation method of (a×b2)/2 where ‘b’ is the smallest diameter and ‘a’ is the largest diameter. Results are shown in FIGS. 14 and 15.

As shown in FIG. 15A, both types of conjugates either reduced tumor size or slowed tumor growth relative to vehicle, which shows that degraders linked to an antibody through the glutarimide ring and degraders linked through the substitution on the terminal phenyl ring are both effective tumor therapies.

As shown in FIG. 15B, treatment with either type of conjugate did not significantly alter group average body weight changes over time relative to the vehicle control group or when compared to initial body weights recorded prior to dosing. Treatment of mice with conjugate did not produce adverse responses and no humane intervention was required during the study. Therefore, treatment with both types of conjugates as described above is well tolerated.

### Example 7-1: Determination of Activity of Conjugates Against In Vivo Myeloid Leukemia Tumor Models

Subcutaneous tumor model—MV-4-11 human acute myeloid leukemia cells (1×10⁷ cells in 0.1 mL) were subcutaneously inoculated into the right flank of female Athymic Nude mice. Mice were treated with each test article when the tumor size reached 100-150 mm³. 10 mg/kg of CD33-D antibody-Compound (XL) conjugate (in 20 mM succinate, 8% sucrose, 0.01% Tween-20 pH 5.5) was delivered by intravenous administration (IV) once a week for 2 weeks. 0.4 mg/kg of Compound (XL) (in 5% NMP, 45% PEG300, 20 mM NaPi pH 6.5) and 10 mg/kg of ARV-825 (as described in Liu, et al. Chemistry & Biology 2015, volume 22, pages 755-763) in 1.5 mg/mL, 5% Kolliphor, HS15 WFI Buffer was treated by intraperitoneal administration (IP) once a day for 14 days. Tumor size and mouse body weight were measured twice per week. Subcutaneous tumor volume (mm³) was calculated with the following formula: (a×b²/2) where ‘b’ is the smallest diameter and ‘a’ is the largest diameter. The study endpoint is that individual mice were euthanized following respective tumor volume ≥1,000 mm³ or day 60, whichever came first.

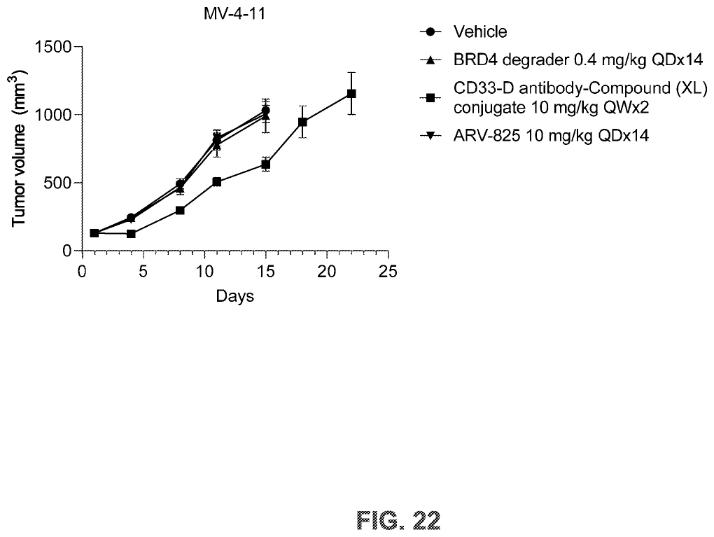

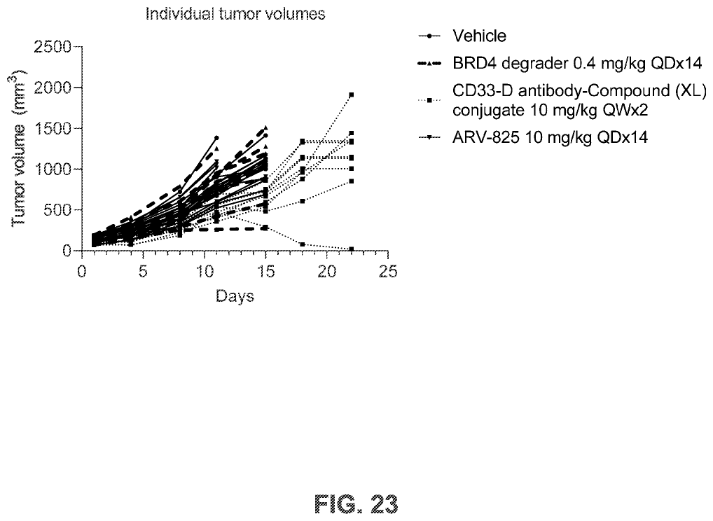

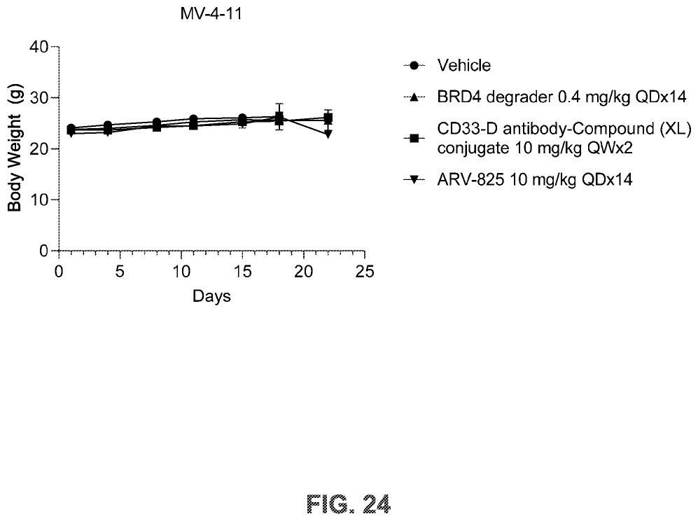

CD33-D antibody-Compound (XL) conjugate (made using a self-immolative spacer and process of the invention) dosed on day 1 and day 8 showed MV-4-11 tumor growth delay over the course of 22 days (FIG. 22). The corresponding BRD4 heterobifunctional degrader small molecule (Compound 15 from Xiamg, W. et al., Biorganic Chemistry 2021, volume 115) dosed daily (equivalent to conjugated payload dose) was inactive. ARV-825, a well profiled BRD4 PROTAC, was also inactive when administered according to its published dosing regimen (https://doi.org/10.3389/fonc.2020.574525; Blood (2016) 128 (22): 748.) FIG. 23 shows individual tumor volumes over time for each dose group and FIG. 24 shows average body weight for mice in each dose group over course of study.

### Example 8: J591 Endogenous Cysteine Conjugation

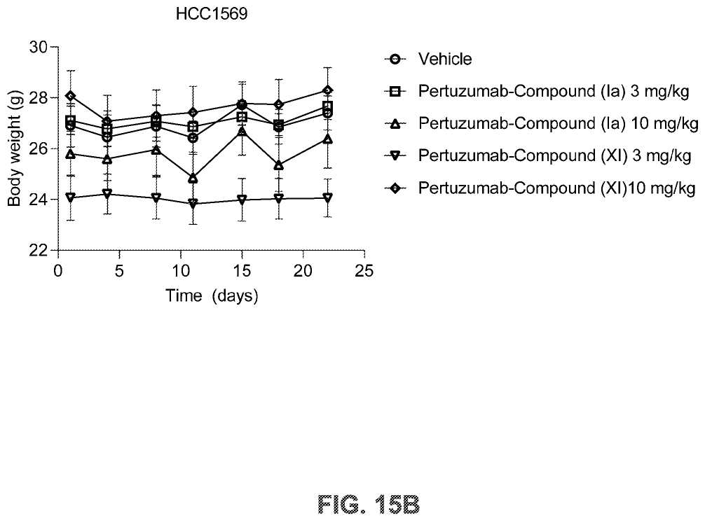

An anti-PSMA antibody with Cys mutation (6.1 mg/mL in 50 mM EPPS, 5 mM EDTA pH 7.0 buffer, was treated with 2.5 equivalents of TCEP and incubated at 37° C. for 2 hours to partially reduce the interchain disulfide bonds. After cooling to ambient temperature, 8 equivalents of Compound (XV) were added as a stock solution in DMA to the reduced antibody to give a final reaction mixture consisting of 5.5 mg/mL reduced antibody+8 equivalents Compound (XV) in 50 mM EPPS, 5 mM EDTA pH 7.0 with 10% (v/v) DMA. The reaction was incubated at ambient temperature for 2 hours. Conjugate was purified into 20 mM succinate, 8% sucrose, 0.01% Tween-20 pH 5.5 by gel filtration using Zeba 40K desalting columns. The conjugate was found to have an average of 4.0 drug/antibody by LC-MS and 25.8% monomer, as shown in FIG. 16.

### Example 9: J591 S239C Site-Specific Engineered Cysteine Conjugation

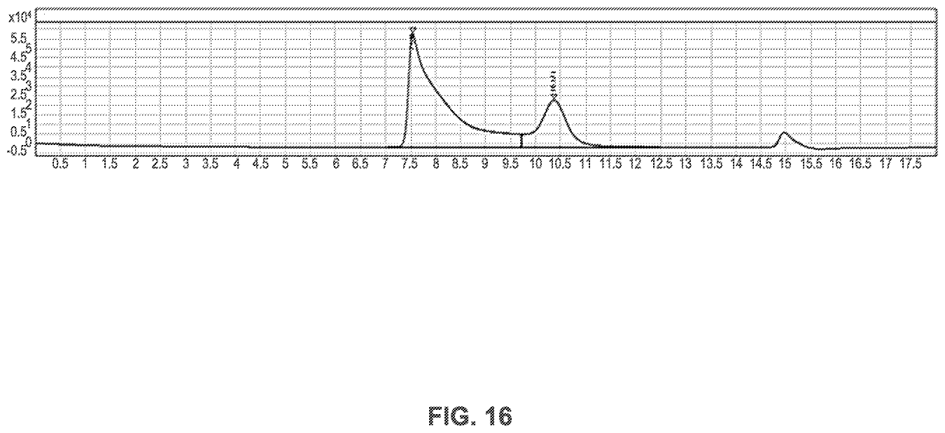

J591 antibody with a S239C mutation in the heavy chain in 20 mM histidine, 250 mM sucrose pH 6.5 was treated with 0.1 volumes of 1M Tris, 200 mM EDTA pH 8.0 to adjust the pH to ˜7.5. The antibody was reduced by treating a 9.4 mg/mL solution with 100 equivalents of DTT and incubating overnight at ambient temperature. The reduced antibody was purified into 50 mM Tris, 100 mM NaCl pH 8.0 by desalting using Zeba desalting columns. The interchain disulfide bonds were reformed by treating a 10 mg/mL solution of reduced antibody with 20 equivalents of dehydroascorbic acid and incubating at ambient temperature for 2 hours to give an antibody intermediate with unpaired cysteines in the heavy chain. This intermediate was purified by SEC using a HiLoad 16/200 Superdex 200 pg column, eluting with 20 mM succinate, 150 mM NaCl, pH 5.5. Conjugation was effected by adjusting the pH of the intermediate to ˜7 with 0.1 volumes of 1M Tris pH 7.5, adding propylene glycol, and adding 6 equivalents of Compound (XV) as a stock solution in DMA such that the final reaction mixture consisted of 2 mg/mL antibody+6 eq. Compound (XV) in 20 mM succinate, 150 mM NaCl adjusted to pH 7.0 with 50% (v/v) propylene glycol cosolvent. The reaction mixture was incubated for 2 hours at ambient temperature. Conjugate was purified into 20 mM succinate, 8% sucrose, 0.01% Tween-20 pH 5.5 by gel filtration using Zeba 40K desalting columns. The conjugate was found to have 1.85 drug/antibody, 96.7% monomer, and <2.5% unconjugated payload, as shown in FIG. 17.

### Example 10: Confirmation of Target Antigen Binding Retention in CD79b-A Antibody-Compound (XLIII) Conjugate

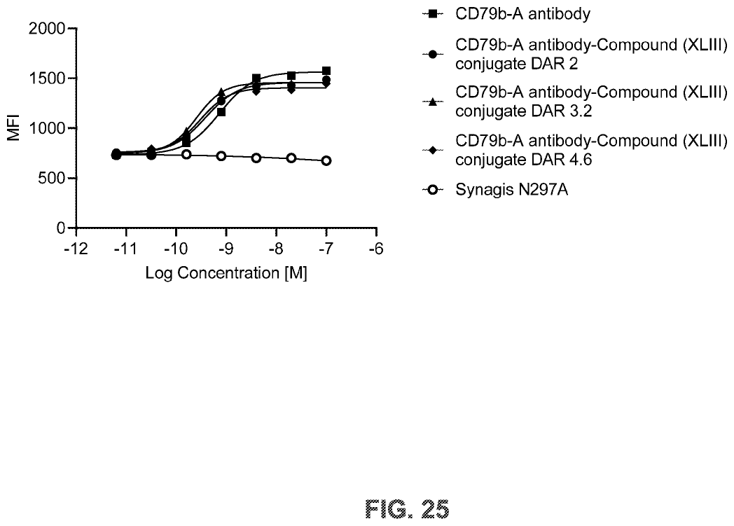

To ensure that the conjugation in the CD79b-A antibody-Compound (XLIII) conjugate did not affect the target antigen binding properties of the antibody portion, CD79b-A antibody-Compound (XLIII) conjugate was tested for its CD79b binding affinity in comparison with CD79b-A antibody (Polatuzumab). Ramos, a B-cell non-Hodgkin lymphoma cell line, was rinsed from its growth medium by centrifugation and resuspension in flow cytometry buffer (5% FBS in PBS). The cells were then seeded in a 96-well plate and incubated with CD79b-A antibody, three different DARs of CD79b-A antibody-Compound (XLIII) conjugate conjugate (2.0, 3.2, and 4.6), or a non-binding control antibody, Synagis N₂₉₇A for 1 hour at 4° C. The cells were washed by centrifugation followed by resuspension in flow cytometry buffer. After two washes, the cells were incubated in the anti-human Alexa Fluor™ 488-conjugated secondary antibody (Invitrogen, A11013) at a concentration of 2 μg/mL for 1 hour at 4° C. The cells were washed twice and analyzed by flow cytometry on the Attune™ NxT Flow Cytometer (ThermoFisher Scientific). As shown in FIG. 25, conjugation was shown to have a negligible effect on antigen binding affinity, as all three DARs of CD79b-A antibody-Compound (XLIII) conjugate conjugate bound to CD79b in a similar manner to CD79b-A antibody. The non-binding control, Synagis N₂₉₇A, showed relatively negligible signal intensity.

### Example 11: Confirmation of IRAK Degradation by CD79b-A Antibody-Compound (XLIII) Conjugate

To show the ability of CD79b-A antibody-Compound (XLIII) conjugate to degrade IRAK4, Ramos cells were seeded in a 6-well cell culture plate and treated with CD79b-A antibody-Compound (XLIII) conjugate at three different DARs (2.0, 3.2, and 4.6) at a concentration range of 0 to 50 nM in growth media (RPMI1640+10% heat-inactivated FBS). Its unconjugated payload, described in WO2021247897 A1, was included for comparison as well as the unconjugated antibody control, CD79b-A antibody. After a 48-hour incubation at 37° C., 5% CO₂, the cells were harvested and rinsed by centrifugation followed by resuspension in ice-cold PBS. After two washes, the cells were pelleted by centrifugation and lysed using RIPA containing protease and phosphatase inhibitors (ThermoFisher Scientific, 78440). The samples were sonicated for a more thorough lysis process. After incubation at 4° C. for 20 minutes, the samples were centrifuged for 20 minutes at 12,000 ref and the supernatant was collected. The protein content in each sample was determined using the Pierce BCA assay kit (ThermoFisher Scientific, 23227) as per the manufacturer's protocol. Bolt™ LDS sample buffer (ThermoFisher Scientific, B0007) and beta-mercaptoethanol (Bio-Rad Laboratories, 1610710) were added for a final v/v ratio of 25% and 2.5%, respectively. A total of 10 μg of protein were added in each well of a 10% polyacrylamide gel (ThermoFisher Scientific, NW00105BOX), and electrophoresis was done for 50 minutes at 150 V. The transfer process was done using the iBlot 2 Dry Blotting System (ThermoFisher Scientific) as per the manufacturer's protocol. After the transfer process, the PVDF membrane was blocked by incubation with 5% skim milk in TBST over 1 hour in RT. The primary antibody for IRAK4 (Abcam, ab119942) was used to stain the membrane for 1 hour in RT, at a dilution ratio of 1:1000 in blocking solution. The membrane was washed 3 times for 5 minutes each in TBST, then stained with an HRP-conjugated secondary antibody (Cell Signaling, 7076) for 1 hour in RT at a dilution of 1:5000. The membrane was imaged using ECL on the iBright™ FL1500 Imaging System (ThermoFisher Scientific). For the loading control, an identical procedure was used but the membrane was stained with an HRP-conjugated anti-3-actin antibody instead (Cell Signaling, 5125) at a dilution of 1:5000.

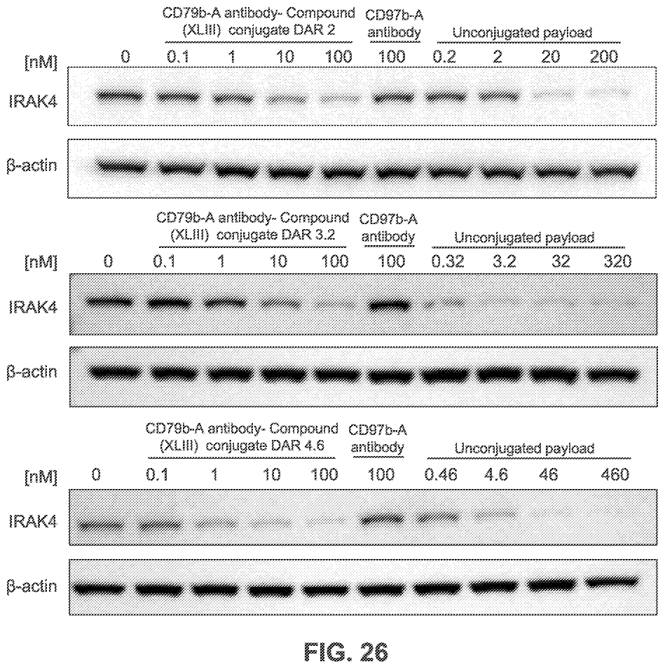

As shown in FIG. 26, cells treated with CD79b-A antibody-Compound (XLIII) conjugate showed dose-dependent degradation of IRAK4, with higher DARs showing more potent degradation. The unconjugated payload of CD79b-A antibody-Compound (XLIII) conjugate also showed dose-dependent degradation of IRAK4. The unconjugated antibody control, CD79b-A antibody, had no effect on IRAK4 levels.

### Example 12: Confirmation of Target Antigen Specific Binding of CD79b-A Antibody-Compound (XLIII) Conjugate

To confirm that the observed degradation of IRAK4 by CD79b-A Antibody-Compound (XLIII) conjugate was mediated by CD79b-binding, a blocking experiment was conducted wherein CD79b-A antibody-Compound (XLIII) conjugate was allowed to compete with CD79b-A antibody for CD79b-binding during the treatment period. The Ramos cell line was seeded in a 12-well cell culture plate and pre-incubated with CD79b-A antibody at 0.05 nM-5 μM for 15 minutes at RT. Without changing the media, CD79b-A antibody-Compound (XLIII) conjugate was added on top at a concentration of 10 nM. Untreated cells, cells treated with only CD79b-A antibody, and cells treated with only CD79b-A antibody-Compound (XLIII) conjugate at 10 nM were added as controls. The cells were incubated for 48 hours in 37° C., 5% CO₂. All cells were harvested at the end of treatment and the level of IRAK4 in each condition was determined using western blot (as described in the previous example) with β-actin as a loading control.

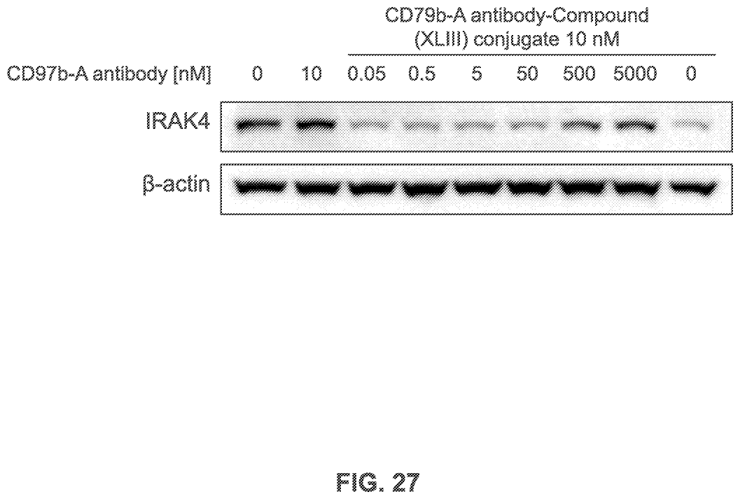

As shown in FIG. 27, antigen-specific binding of CD79b-A antibody-Compound (XLIII) conjugate was confirmed by an increase in the IRAK4 band intensity with increasing CD79b-A antibody concentration in the co-treated condition. At 5 μM of CD79b-A antibody, CD79b-A antibody-Compound (XLIII) conjugate had almost no effect on IRAK4, as evident by a band intensity similar to the untreated condition. The cells treated with 10 nM of CD79b-A antibody did not show IRAK4 degradation and the cells treated with only CD79b-A antibody-Compound (XLIII) conjugate showed the most potent degradation.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A conjugate of formula (XXXII): 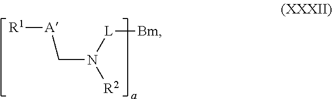
or a pharmaceutically acceptable salt thereof, wherein:
a is from 1 to 10;
A′ is 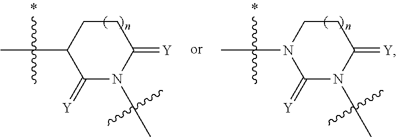
wherein
n is 0 or 1;
each Y is independently S or 0; [symbol]
indicates the point of attachment to R¹; and [symbol]
indicates the point of attachment to the methylene group;
R¹, together with A′, is a compound that induces a protein-protein interaction;
R² is selected from hydrogen, a group that provides stability to R¹-A′, a group that provides solubility to R¹-A′, and a group that provides stability and solubility to R¹-A′;
L is a cleavable linker; and
Bm is a binding moiety that is capable of specifically binding to a protein.

2. A conjugate of formula (XX): 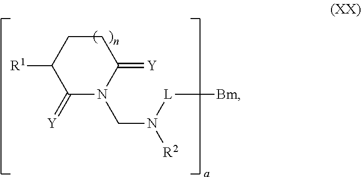
or a pharmaceutically acceptable salt thereof, wherein:
a is from 1 to 10;
n is 0 or 1;
R¹ is a compound that induces a protein-protein interaction;
R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;
each Y is independently S or 0;
L is a cleavable linker; and
Bm is a binding moiety that is capable of specifically binding to a protein.

3. The conjugate of claim 1 or 2, or a pharmaceutically acceptable salt thereof, wherein the binding moiety is an antibody, antibody fragment, or an antigen-binding fragment.

4. The conjugate of any one of claims 1 to 3, or a pharmaceutically acceptable salt thereof, wherein a is from 2 to 8.

5. The conjugate of any one of claims 1 to 4, or a pharmaceutically acceptable salt thereof, wherein the linker is cleavable by a protease.

6. The conjugate of claim 5, or a pharmaceutically acceptable salt thereof, wherein L is selected from the group consisting of 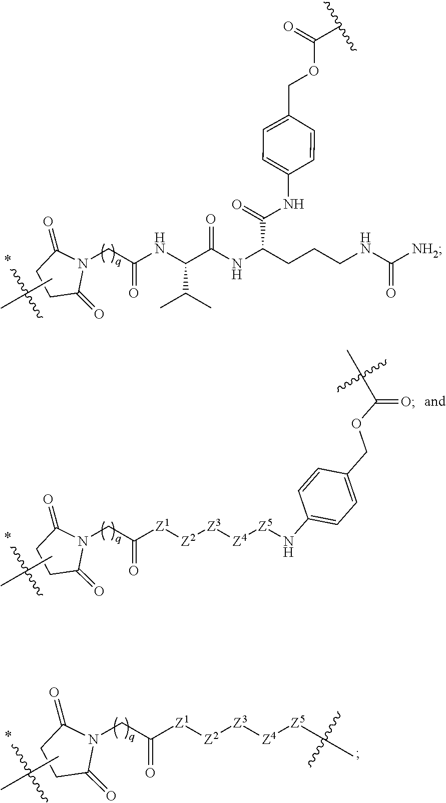
wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues; [symbol]
is the point of attachment to the parent molecular moiety; and [symbol]
is the point of attachment to the binding moiety.

7. The conjugate of claim 6, or a pharmaceutically acceptable salt thereof, wherein Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

8. The conjugate of claim 7, or a pharmaceutically acceptable salt thereof, wherein:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine;
Z⁴ is selected from the group consisting of L-citrulline, D-citrulline, L-asparagine, D-asparagine, L-lysine, D-lysine, L-phenylalamine, D-phenylalanine, and glycine; and
Z⁵ is absent or glycine.

9. The conjugate of any one of claims 1 to 8, or a pharmaceutically acceptable salt thereof, wherein L is

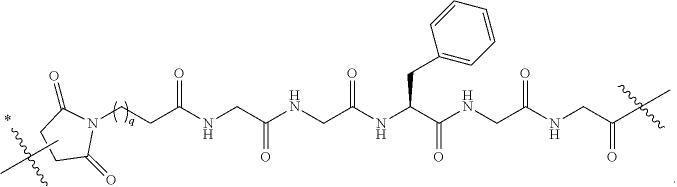

10. The conjugate of claim 9, or a pharmaceutically acceptable salt thereof, wherein q is 4.

11. The conjugate of any one of claims 1 to 4, or a pharmaceutically acceptable salt thereof, wherein L is a bioreducible linker.

12. The conjugate of claim 11, or a pharmaceutically acceptable salt thereof, wherein L is selected from the group consisting of 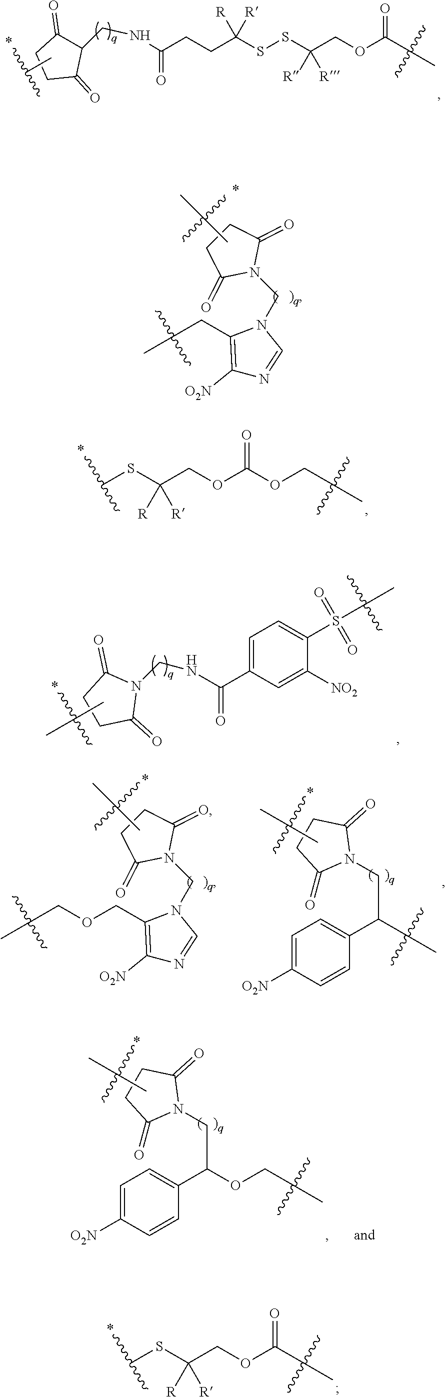
wherein:
q is from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; [symbol]
is the point of attachment to the parent molecular moiety; and [symbol]
is the point of attachment to the binding moiety.

13. The conjugate of claim 12, or a pharmaceutically acceptable salt thereof, wherein L is

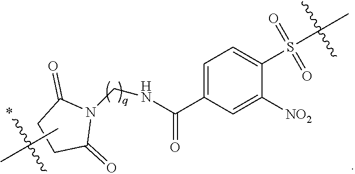

14. The conjugate of claim 13, or a pharmaceutically acceptable salt thereof, wherein q is 2.

15. The conjugate of any one of claims 1 to 4, or a pharmaceutically acceptable salt thereof, wherein L is a click-to-release linker.

16. The conjugate of claim 15, or a pharmaceutically acceptable salt thereof, wherein L is 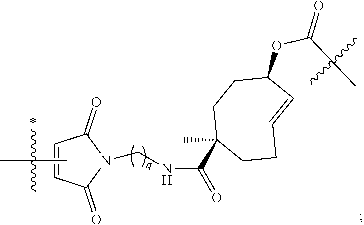
wherein:
q is from 2 to 10; [symbol]
is the point of attachment to the parent molecular moiety; and [symbol]
is the point of attachment to the binding moiety.

17. The conjugate of any one of claims 1 to 4, or a pharmaceutically acceptable salt thereof, wherein L is a beta-glucuronidase cleavable linker.

18. The conjugate of claim 15, or a pharmaceutically acceptable salt thereof, wherein L is 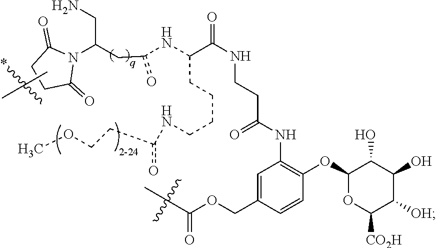
wherein:
q is from 2 to 10;
- - - is absent or a bond; [symbol]
is the point of attachment to the parent molecular moiety; and [symbol]
is the point of attachment to the binding moiety.

19. The conjugate of any one of claims 1 to 18, or a pharmaceutically acceptable salt thereof, wherein L is attached to a cysteine, lysine, tyrosine, or glutamine in the Bm.

20. The conjugate of claim 19, or a pharmaceutically acceptable salt thereof, wherein the cysteine or lysine is an engineered cysteine or lysine.

21. The conjugate of claim 19, or a pharmaceutically acceptable salt thereof, wherein the cysteine or lysine is endogenous to the Bm.

22. The conjugate of any one of claims 1 to 21, or a pharmaceutically acceptable salt thereof, wherein Bm is an antibody or antigen binding portion thereof.

23. The conjugate of claim 22, wherein L is attached to an engineered cysteine at heavy chain position S239 and/or K334 of the antibody or antigen binding portion thereof according to EU numbering.

24. The conjugate of claim 22, wherein L is attached to the glutamine at heavy chain position 295 of the antibody or antigen binding portion thereof according to EU numbering.

25. The conjugate of any one of claims 1 to 24, or a pharmaceutically acceptable salt thereof, wherein the protein that the Bm binds to is a surface antigen, optionally wherein binding of the Bm to the surface antigen results in internalization of the conjugate or pharmaceutically acceptable salt thereof into a cell.

26. The conjugate of claim 25, or a pharmaceutically acceptable salt thereof, wherein the surface antigen comprises 5T4, ACE, ADRB3, AKAP-4, ALK, AOC3, APP, Axin1, AXL, B7H3, B7-H4, BCL2, BCMA, bcr-ab1, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor, IL-4 receptor, IL-13Ra2, IL-1 1Ra, IL-1 receptor, IL-12 receptor, IL-23 receptor, IL-13 receptor, IL-22 receptor, IL-4 receptor, IL-5 receptor, IL-6 receptor, interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-1a, LAIR1, LAMP-1, LCK, Legumain, LewisY, LFA-I (CDI 1a), L-selectin (CD62L), LILRA2, LIV-1, LMP2, LRRC15, LY6E, LY6K, LY75, MAD-CT-1, MAD-CT-2, MAGE A1, MelanA/MARTl, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PD-1, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRP, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof, optionally wherein the Bm that binds to CD33 comprises the amino acid sequences of SEQ ID NOs:1 and 2, 3 and 4, 5 and 6, 22, and 23, 24 and 25, or 27 and 28, wherein the Bm that binds to PSMA comprises the amino acid sequences of SEQ ID NOs:7 and 8, 9 and 10, 11 and 12, or 29 and 30, wherein the Bm that binds to HER2 comprises the amino acid sequences of SEQ ID NOs:13 and 14, 15 and 16, or 17, and 18, wherein the Bm that binds to CD20 comprises the amino acid sequences of SEQ ID NOs:31 and 32, wherein the Bm that binds to CD79b comprises the amino acid sequences of SEQ ID NO:33 and 34, or wherein the Bm that binds to BCMA comprises the amino acid sequences of SEQ ID NOs:35 and 36.

27. The conjugate of claim 25, or a pharmaceutically acceptable salt thereof, wherein the surface antigen comprises HER2, CD20, CD38, CD33, BCMA, CD138, EGFR, FGFR4, GD2, PDGFR, or combinations thereof.

28. The conjugate of claim 22, or a pharmaceutically acceptable salt thereof, wherein the antibody is selected from the group consisting of rituximab, trastuzumab, gemtuzumab, pertuzumab, obinutuzumab, ofatumumab, daratumumab, STI-6129, lintuzumab, huMy9-6, belantamab, indatuximab, cetuximab, dinutuximab, anti-CD38 A2 antibody, huAT15/3 antibody, alemtuzumab, ibritumomab, tositumomab, bevacizumab, panitumumab, tremelimumab, ticilimumab, catumaxomab, oregovomab, veltuzumab, polatuzumab, J591, lorvotuzumab and sacituzumab.

29. The conjugate of claim 28, or a pharmaceutically acceptable salt thereof, wherein the antibody is rituximab, trastuzumab, pertuzumab, huMy9-6, lintuzumab, gemtuzumab, or CD33-D.

30. The conjugate of any one of claim 1 or 3 to 29, or a pharmaceutically acceptable salt thereof, wherein R² is a group that provides stability to the conjugate.

31. The conjugate of any one of claim 1 or 3 to 30, or a pharmaceutically acceptable salt thereof, wherein R² is selected from C₂-C₆alkenyl, C₁-C₆alkyl; C₂-C₆alkynyl, benzyl, C₃-C₆cycloalkyl, and C₃-C₆cycloalkyl(C₁-C₃alkyl).

32. The conjugate of any one of claim 1 or 3 to 31, or a pharmaceutically acceptable salt thereof, wherein R² is C₁-C₆alkyl.

33. The conjugate of any one of claim 1 or 3 to 32, or a pharmaceutically acceptable salt thereof, wherein R² is methyl.

34. The conjugate of any one of claim 1 or 3 to 29, or a pharmaceutically acceptable salt thereof, wherein R² is a group that provides solubility to the conjugate.

35. The conjugate of any one of claims 1, 3 to 29, or 34, or a pharmaceutically acceptable salt thereof, wherein R² is selected from: 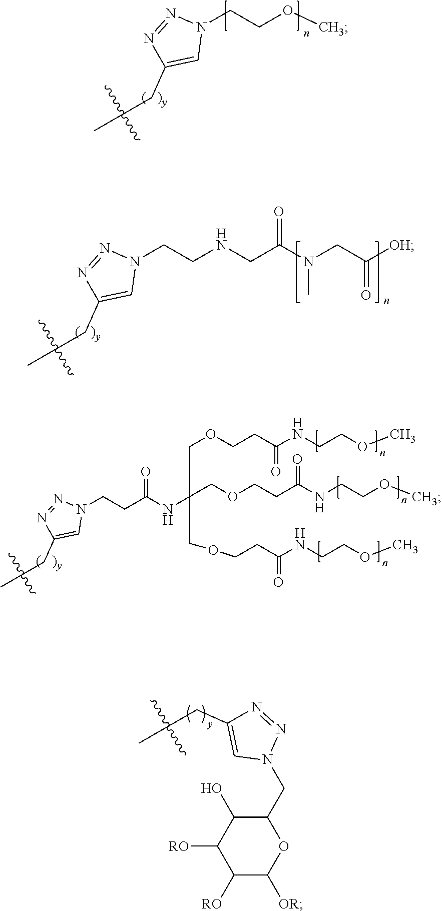 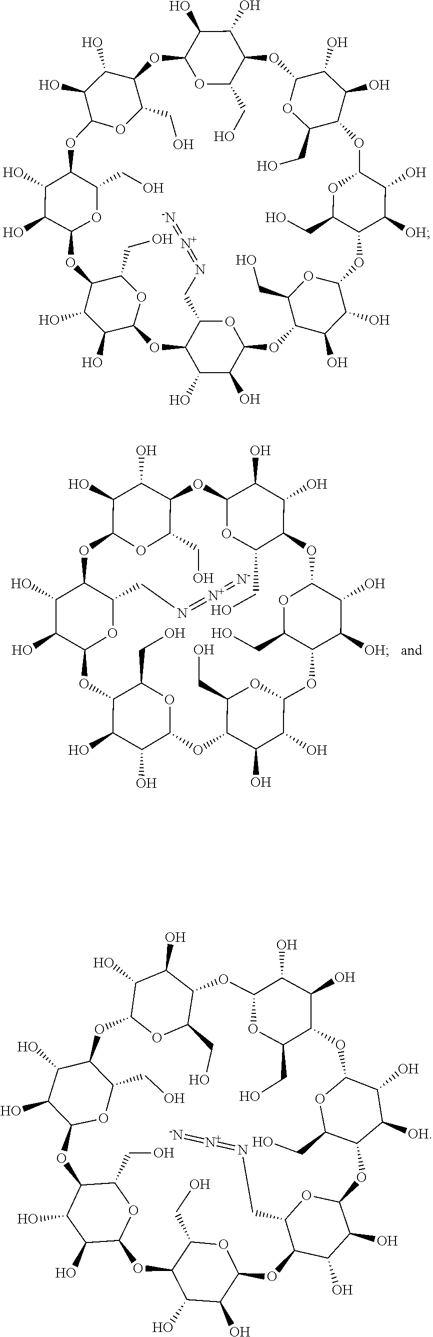
wherein:
each n is independently 1, 2, 3, 4, or 5;
each y is independently 1 or 2; and
each R is independently hydrogen, C₆H₁₁O₅, C₁₂H₂₁O₁₀, C₁₈H₃₁O₁₅, or C₂₄H₄₁O₂₀.

36. The conjugate of any one of claims 1 to 35, or a pharmaceutically acceptable salt thereof, wherein each Y is O.

37. The conjugate of any one of claim 1 or 3 to 36, or a pharmaceutically acceptable salt thereof, wherein R¹-A′ is a PPI modulator.

38. The conjugate of any one of claim 1 or 3 to 36, or a pharmaceutically acceptable salt thereof, wherein R¹-A′ is a targeted protein degrader.

39. The conjugate of any one of claim 1, or 3 to 38, or a pharmaceutically acceptable salt thereof, wherein R¹-A′ is a molecular glue.

40. The conjugate of any one of claim 1, or 3 to 39, or a pharmaceutically acceptable salt thereof, wherein R¹-A′ is a substituted isoindoline.

41. The conjugate of any one of claim 1, or 3 to 40, or a pharmaceutically acceptable salt thereof, wherein R¹-A′ is a 5′-substituted isoindoline.

42. The conjugate of any one of claims 1 to 41, or a pharmaceutically acceptable salt thereof, wherein R¹ is a compound of formula (XXX): 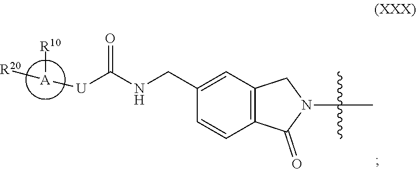
wherein: [symbol]
denotes the point of attachment to the parent molecular moiety;
A is phenyl or a C₄-C₁₀cycloalkyl ring;
R¹⁰ is independently selected from hydrogen and halo;
U is selected from NH and CF₂; and
R²⁰ is selected from —CH₃, —C(O)R³, —N(R⁴)₂, —(CH₂)ₙOH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
R³ is hydrogen or C₁-C₆alkyl;
each R⁴ is independently hydrogen or C₁-C₆alkyl;
Q′ is O, S, or NR⁴;
n is 1-6; and
m is 2-5.

43. The conjugate of claim 42, or a pharmaceutically acceptable salt thereof, wherein
A is phenyl;
U is NH;
R¹⁰ is halo; and
R²⁰ is methyl.

44. The conjugate of claim 42, or a pharmaceutically acceptable salt thereof, wherein
A is phenyl;
U is NH;
R¹⁰ is halo; and
R²⁰ is —(CH₂)₂O(CH₂)₂NHCH₃.

45. The conjugate of any one of claim 1, or 3 to 38, or a pharmaceutically acceptable salt thereof, wherein R¹-A′ is a proteolysis targeting chimera (PROTAC).

46. The conjugate of any one of claims 1 to 38 or 45, or a pharmaceutically acceptable salt thereof, wherein R¹ has the formula:
POI-L¹⁰⁰-CBN;
wherein:
POI is a compound that binds to a protein of interest;
L¹⁰⁰ is a PROTAC linker; and
CBN is a cereblon binding moiety.

47. The conjugate of claim 46, or a pharmaceutically acceptable salt thereof, wherein the protein of interest is a nuclear hormone receptor, a translation termination factor, a transcription factor, a cyclin-dependent kinase, a tyrosine kinase, a serine/threonine kinase, or an E3 ligase.

48. The conjugate of claim 46 or 47, or a pharmaceutically acceptable salt thereof, wherein the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

49. The conjugate of any one of claims 46 to 48, or a pharmaceutically acceptable salt thereof, wherein L¹⁰⁰ comprises one or more functional groups selected from glycol, alkyl, alkynyl, triazolyl, piperazinyl, piperidinyl, and combinations thereof.

50. The conjugate of any one of claims 46 to 49, or a pharmaceutically acceptable salt thereof, wherein CBN is selected from 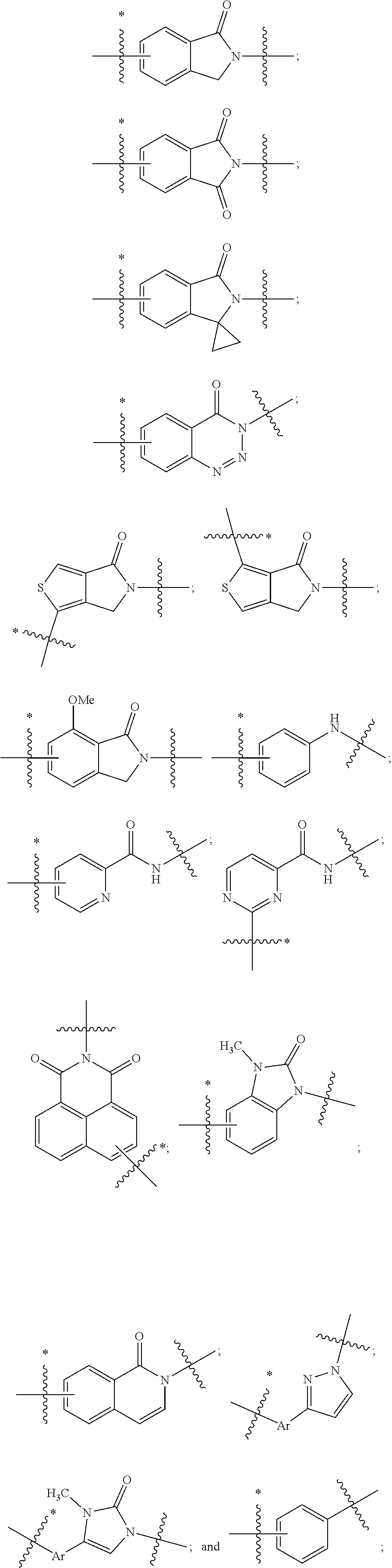
wherein [symbol]
indicates the point of attachment to A′; and [symbol]
indicates the point of attachment to L¹⁰⁰.

51. The conjugate of any one of claims 1 to 36, 38, or 45 to 50, or a pharmaceutically acceptable salt thereof, wherein R¹ is selected from 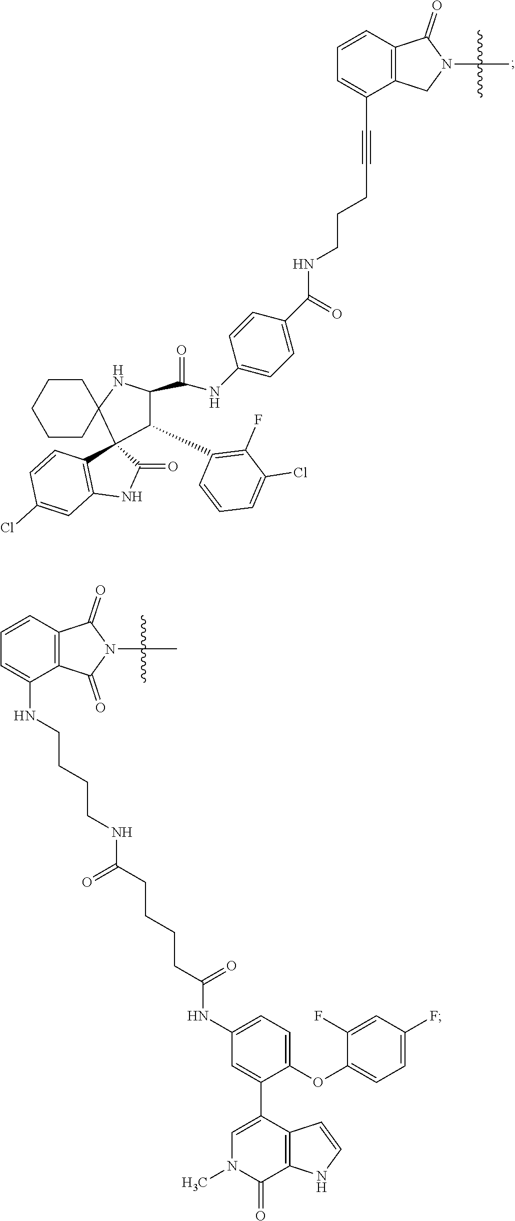 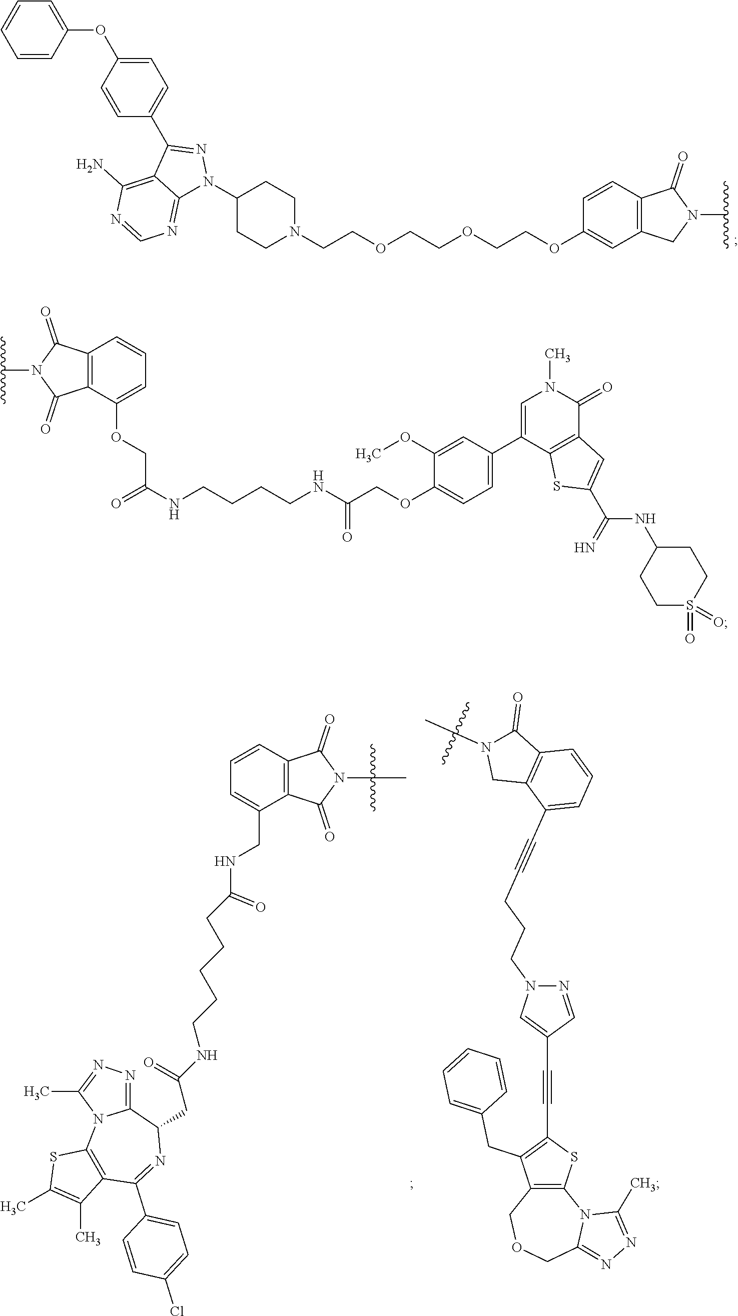 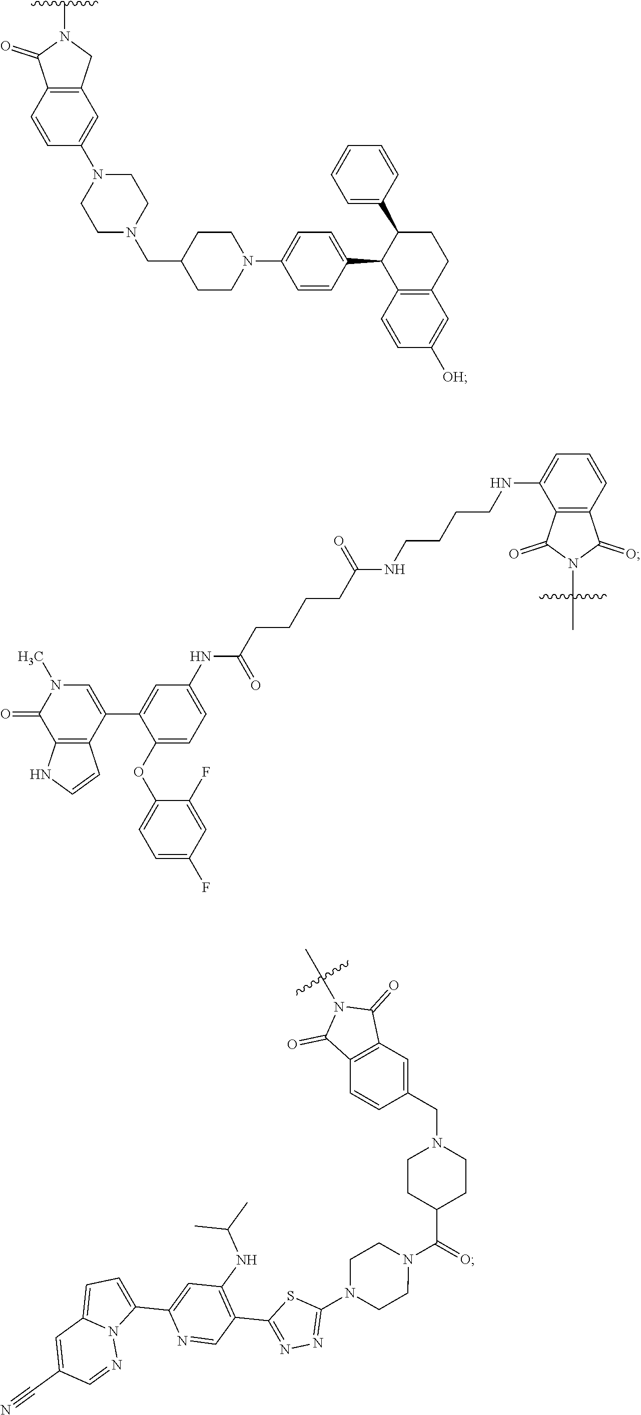 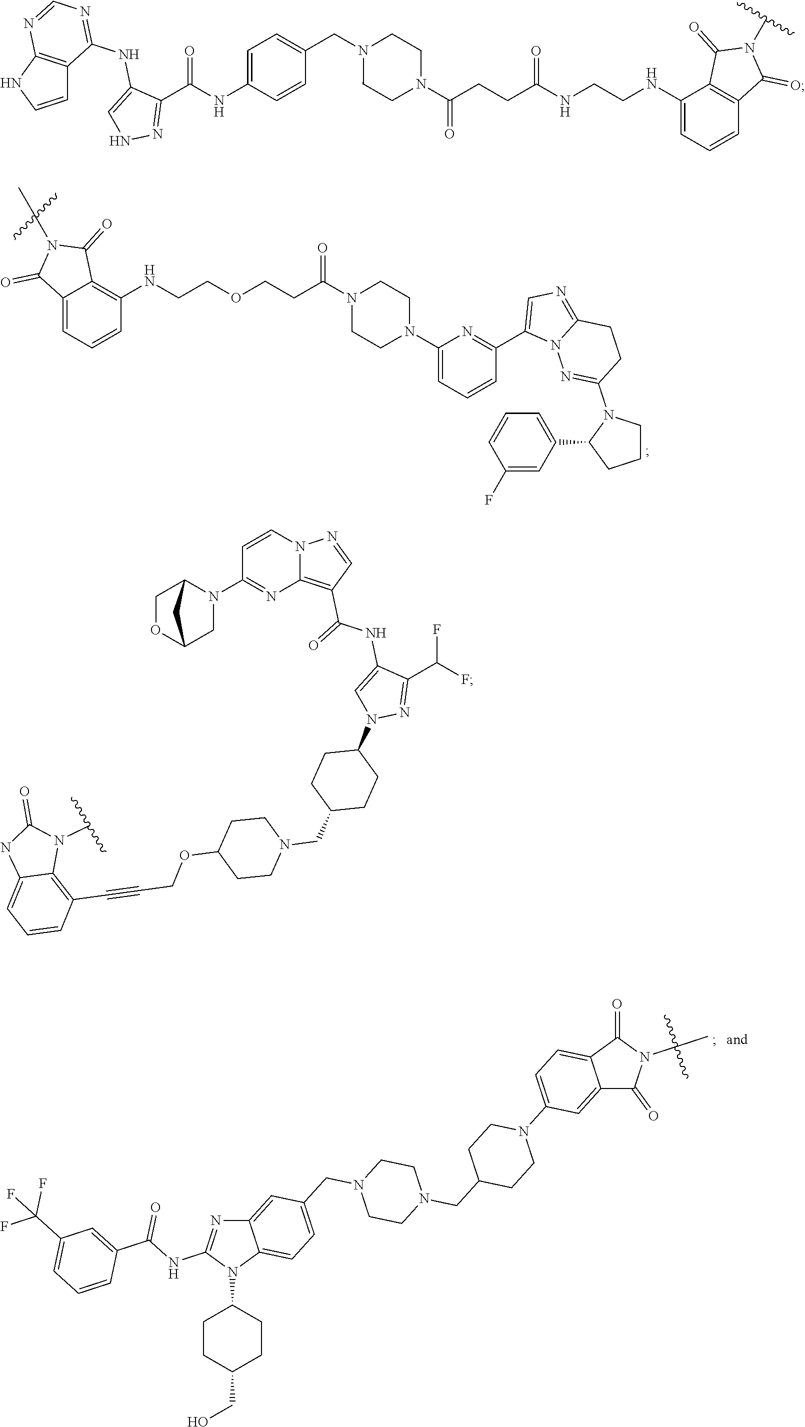 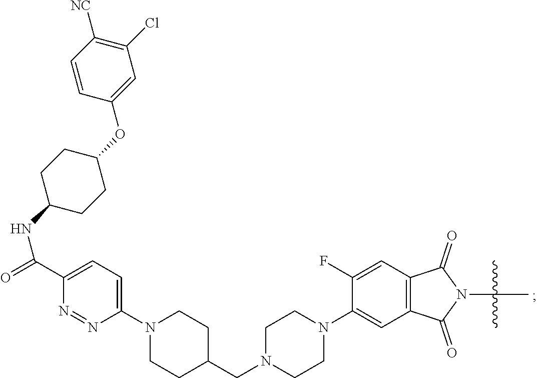
wherein [symbol] denotes the point of attachment to A′.

52. The conjugate of any one of claims 1 to 51, or a pharmaceutically acceptable salt thereof, wherein (a) the protein that the Bm binds to is CD33 and R¹ binds to mouse double minute 2 homolog (MDM2), (b) the protein that the Bm binds to is prostate specific membrane antigen (PSMA) and R¹ binds to androgen receptor (AR), (c) the protein that the Bm binds to is CD33 and R¹ binds to bromodomain-containing protein 4 (BRD4), (d) the protein that the Bm binds to is HER2 and R¹ binds to G1 to S Phase Transition 1 (GSPT1), (e) the protein that the Bm binds to is CD33 and R¹ binds to GSPT1, (f) the protein that the Bm binds to is CD79b and R¹ binds to IRAK4, (g) the protein that the Bm binds to is HER2 and R¹ binds to BRD4, (h) the protein that the Bm binds to is BCMA and R¹ binds to BRD4, or (i) the protein that the Bm binds to is HER2 and R¹ binds to ER.

53. A pharmaceutical composition comprising a conjugate of any one of claims 1 to 52, or a pharmaceutically acceptable salt thereof, and one or more pharmaceutically acceptable carriers.

54. A method of treating cancer in a subject in need thereof, the method comprising administering to the subject a pharmaceutically acceptable amount of a conjugate or composition of any of claims 1 to 53, or a pharmaceutically acceptable salt thereof.

55. The method of claim 54, wherein the cancer is breast cancer, gastric cancer, lymphoma, acute myeloid leukemia, multiple myeloma, head and neck cancer, squamous cell carcinoma, hepatocellular carcinoma, prostate cancer, non-small cell lung cancer, colon cancer, ovarian cancer, neuregulin-1 (NRG1)-positive cancer, lung cancer, or non-Hodgkin lymphoma.

56. The method of claim 54, wherein (a) the protein that the Bm binds to is CD33, R¹ binds to MDM2, and the cancer is acute myeloid leukemia, (b) the protein that the Bm binds to is prostate specific membrane antigen (PSMA), R¹ binds to androgen receptor (AR), and the cancer is prostate cancer, (c) the protein that the Bm binds to is CD33, R¹ binds to bromodomain-containing protein 4 (BRD4), and the cancer is acute myeloid leukemia, (d) the protein that the Bm binds to is HER2, R¹ binds to G1 to S Phase Transition 1 (GSPT1), and the cancer is breast cancer, gastric cancer, non-small cell lung cancer, bile duct cancer, colon cancer, ovarian cancer, or neuregulin-1 (NRG1)-positive cancer, (e) the protein that the Bm binds to is CD79b, R¹ binds to IRAK4, and the cancer is a non-Hodgkin lymphoma, (f) the protein that the Bm binds to is HER2, R¹ binds to BRD4, and and the cancer is breast cancer, gastric cancer, non-small cell lung cancer, bile duct cancer, colon cancer, ovarian cancer, or neuregulin-1 (NRG1)-positive cancer, or (g) the protein that the Bm binds to is BCMA, R¹ binds to BRD4, and and the cancer is multiple myeloma.

57. The method of claim any one of claims 54 to 56, further comprising administering to the subject a pharmaceutically acceptable amount of an additional agent prior to, after, or simultaneously with the conjugate or composition of any one of claims 1 to 53, or a pharmaceutically acceptable salt thereof.

58. The method of claim 57, wherein the additional agent is a cytotoxic agent or an immune response modifier.

59. The method of claim 58, wherein the immune response modifier is a checkpoint inhibitor.

60. The method of claim 59, wherein the checkpoint inhibitor comprises a PD-1 inhibitor, a PD-L1 inhibitor, a CTLA-4 inhibitor, a TIM3 inhibitor, and/or a LAG-3 inhibitor.

61. A compound of formula (XXII): 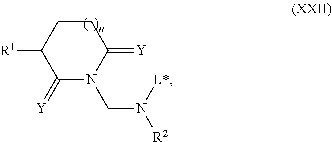
or a pharmaceutically acceptable salt thereof, wherein:
n is 0 or 1;
R¹ is a compound that induces a protein-protein interaction;
R² is selected from hydrogen, —(CH₂CH₂O)ᵥ—CH₃, C₂-C₆alkenyl, C₁-C₆alkyl; C₂-C₆alkynyl, benzyl, C₃-C₆cycloalkyl, and C₃-C₆cycloalkyl(C₁-C₃alkyl), wherein v is from 1 to 24;
each Y is independently S or O; and
L* is a cleavable linker precursor that conjugates to the binding moiety.

62. A compound of formula (XXXI): 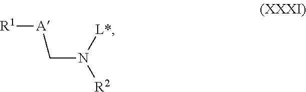
or a pharmaceutically acceptable salt thereof, wherein:
A′ is 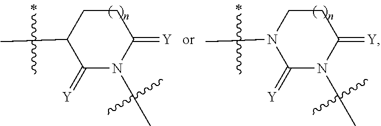
wherein
n is 0 or 1;
each Y is independently S or 0; [symbol]
indicates the point of attachment to R¹; and [symbol]
indicates the point of attachment to the methylene group;
R¹, together with A′, is a compound that induces a protein-protein interaction;
R² is selected from hydrogen, a group that provides stability to R¹-A′, a group that provides solubility to R¹-A′, and a group that provides stability and solubility to R¹-A′; and
L is a cleavable linker precursor that conjugates to the binding moiety.

63. The compound of claim 61 or 62, or a pharmaceutically acceptable salt thereof, wherein L* is a protease cleavable linker precursor.

64. The compound of any one of claims 61 to 63, or a pharmaceutically acceptable salt thereof, wherein L* is selected from the group consisting of 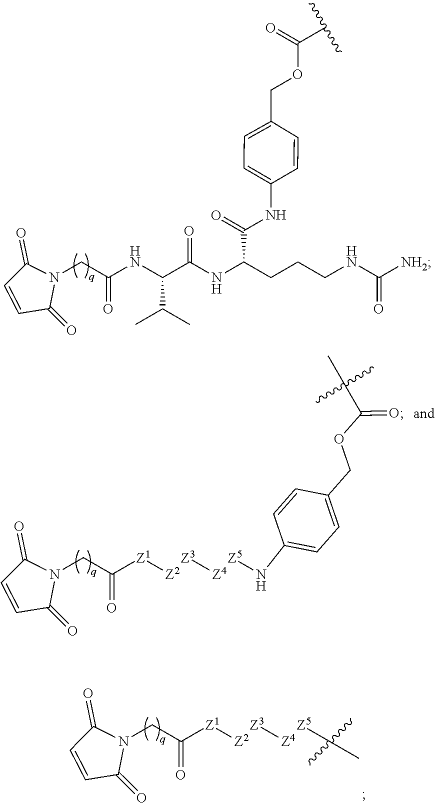
wherein:
q is from 2 to 10;
Z¹, Z², Z³, Z⁴, and Z⁵ are each independently absent or a naturally-occurring amino acid residue in the L- or D-configuration, provided that at least two of Z¹, Z², Z³, Z⁴, and Z⁵ are amino acid residues; and [symbol]
is the point of attachment to the parent molecular moiety.

65. The compound of claim 64, or a pharmaceutically acceptable salt thereof, wherein Z¹, Z², Z³, Z⁴, and Z⁵ are independently absent or selected from the group consisting of L-valine, D-valine, L-citrulline, D-citrulline, L-alanine, D-alanine, L-glutamine, D-glutamine, L-glutamic acid, D-glutamic acid, L-aspartic acid, D-aspartic acid, L-asparagine, D-asparagine, L-phenylalanine, D-phenylalanine, L-lysine, D-lysine, and glycine; provided that at least two of Z¹, Z², Z³, Z⁴, and Z⁵ are amino acid residues.

66. The compound of claim 65, or a pharmaceutically acceptable salt thereof, wherein:
Z¹ is absent or glycine;
Z² is absent or selected from the group consisting of L-glutamine, D-glutamine, L-glutamic acid, D-glutamic acid, L-aspartic acid, D-aspartic acid, L-alanine, D-alanine, and glycine;
Z³ is selected from the group consisting of L-valine, D-valine, L-alanine, D-alanine, L-phenylalanine, D-phenylalanine, and glycine;
Z⁴ is selected from the group consisting of L-citrulline, D-citrulline, L-asparagine, D-asparagine, L-lysine, D-lysine, L-phenylalamine, D-phenylalanine, and glycine; and
Z⁵ is absent or glycine.

67. The compound of any one of claims 61 to 66, or a pharmaceutically acceptable salt thereof, wherein L* is 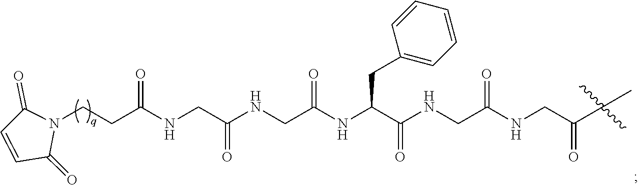
wherein [symbol] is the point of attachment to the parent molecular moiety.

68. The compound of claim 67, or a pharmaceutically acceptable salt thereof, wherein q is 4.

69. The compound of claim 61 or 62, or a pharmaceutically acceptable salt thereof, wherein L* is a bioreducible linker precursor.

70. The compound of any one of claims 61, 62, or 69, or a pharmaceutically acceptable salt thereof, wherein L* is selected from the group consisting of 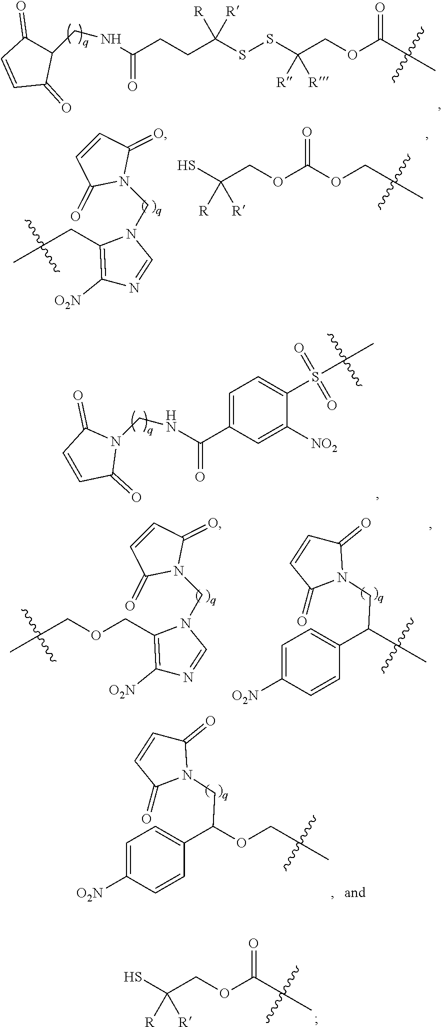
wherein:
q is from 2 to 10;
R, R′, R″, and R′″ are each independently selected from hydrogen, C₁-C₆alkoxyC₁-C₆alkyl, (C₁-C₆)₂NC₁-C₆alkyl, and C₁-C₆alkyl, or, two geminal R groups, together with the carbon atom to which they are attached, can form a cyclobutyl or cyclopropyl ring; [symbol]
is the point of attachment to the parent molecular moiety.

71. The compound of claim 70, or a pharmaceutically acceptable salt thereof, wherein L* is

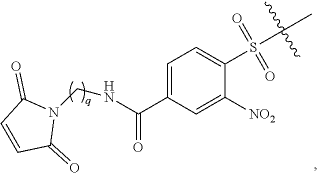

72. The compound of claim 71, or a pharmaceutically acceptable salt thereof, wherein q is 2.

73. The compound of claim 61 or 62, or a pharmaceutically acceptable salt thereof, wherein L* is a click-to-release linker precursor.

74. The compound of claim 73, or a pharmaceutically acceptable salt thereof, wherein L* is 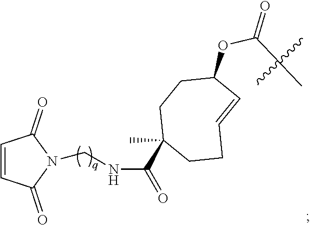
wherein:
q is from 2 to 10; and [symbol]
is the point of attachment to the parent molecular moiety.

75. The compound of claim 61 or 62, or a pharmaceutically acceptable salt thereof, wherein L* is a beta-glucuronidase cleavable linker precursor.

76. The compound of claim 75, or a pharmaceutically acceptable salt thereof, wherein L* is 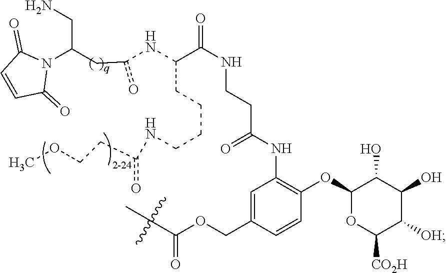
wherein:
q is from 2 to 10;
- - - is absent or a bond; and [symbol]
is the point of attachment to the parent molecular moiety.

77. The compound of any one of claims 62 to 76, or a pharmaceutically acceptable salt thereof, wherein R² is a group that provides stability to R¹-A′.

78. The compound of any one of claims 62 to 77, or a pharmaceutically acceptable salt thereof, wherein R² is selected from C₂-C₆alkenyl, C₁-C₆alkyl; C₂-C₆alkynyl, benzyl, C₃-C₆cycloalkyl, and C₃-C₆cycloalkyl(C₁-C₃alkyl),

79. The compound of any one of claims 62 to 78, or a pharmaceutically acceptable salt thereof, wherein R² is C₁-C₆alkyl.

80. The compound of any one of claims 62 to 79, or a pharmaceutically acceptable salt thereof, wherein R² is methyl.

81. The compound of any one of claims 62 to 76, or a pharmaceutically acceptable salt thereof, wherein R² is a group that provides solubility to R¹-A′.

82. The conjugate of any one of claims 62 to 76 or 81, or a pharmaceutically acceptable salt thereof, wherein R² is selected from: 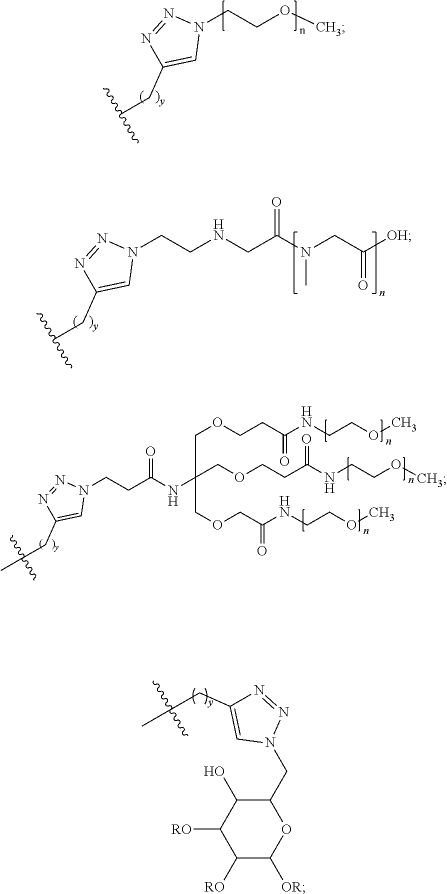 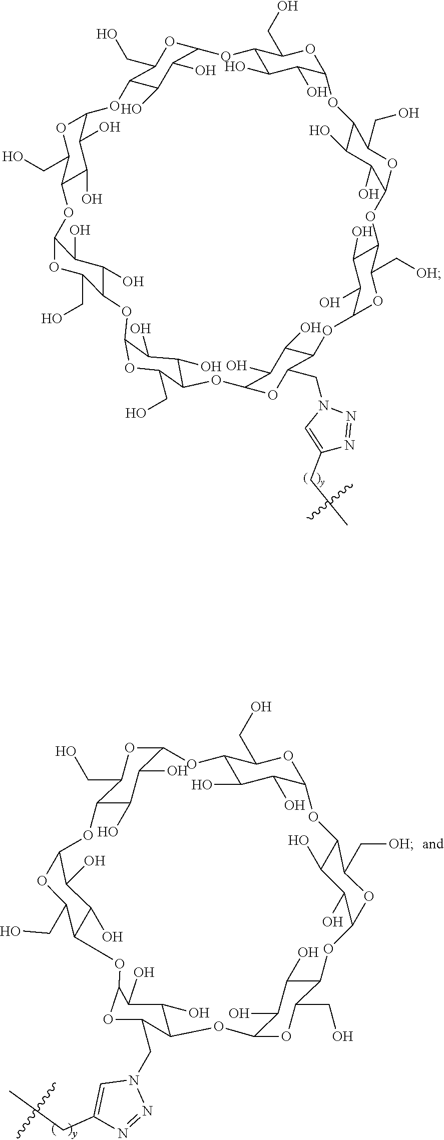 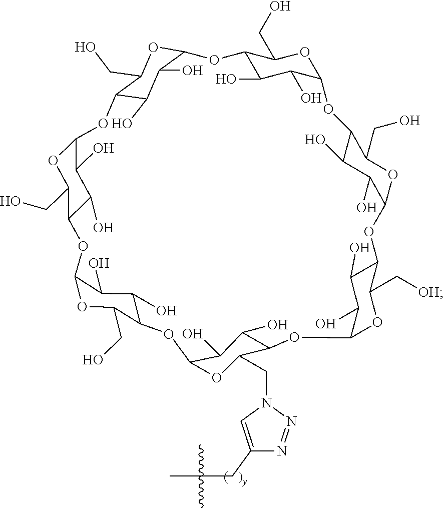
wherein:
each n is independently 1, 2, 3, 4, or 5;
each y is independently 1 or 2;
each R is independently hydrogen, C₆H₁₁O₅, C₁₂H₂₁O₁₀, C₁₈H₃₁O₁₅, or C₂₄H₄₁O₂₀.

83. The compound of any one of claims 61 to 82, or a pharmaceutically acceptable salt thereof, wherein each Y is O.

84. The compound of any one of claims 62 to 83, or a pharmaceutically acceptable salt thereof, wherein R¹-A′ is a PPI modulator.

85. The compound of any one of claims 62 to 83, or a pharmaceutically acceptable salt thereof, wherein R¹-A′ is a targeted protein degrader.

86. The compound of any one of claims 62 to 83, or a pharmaceutically acceptable salt thereof, wherein R¹-A′ is a molecular glue.

87. The compound of any one of claims 62 to 86, or a pharmaceutically acceptable salt thereof, wherein R¹-A′ is a substituted isoindoline.

88. The compound of any one of claims 62 to 87, or a pharmaceutically acceptable salt thereof, wherein R¹-A′ is a 5′-substituted isoindoline.

89. The compound of any one of claims 61 to 88, or a pharmaceutically acceptable salt thereof, wherein R¹ has the formula: 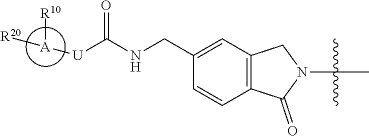
wherein: [symbol]
denotes the point of attachment to the parent molecular moiety;
A is phenyl or a C₄-C₁₀cycloalkyl ring;
R¹⁰ is independently selected from hydrogen and halo;
U is selected from NH and CF₂; and
R²⁰ is selected from —CH₃, —C(O)R³, —N(R⁴)₂, —(CH₂)ₙOH, —(CH₂)ₙN(R⁴)₂, —(CH₂)ₙQ′(CH₂)ₘOH, —(CH₂)ₙQ′(CH₂)ₘSH, and —(CH₂)ₙQ′(CH₂)ₘN(R⁴)₂; wherein
R³ is hydrogen or C₁-C₆alkyl;
each R⁴ is independently hydrogen or C₁-C₆alkyl;
Q′ is O, S, or NR⁴;
n is 1-6; and
m is 2-5.

90. The compound of claim 89, or a pharmaceutically acceptable salt thereof, wherein
A is phenyl;
U is NH;
R¹⁰ is halo; and
R²⁰ is methyl.

91. The compound of claim 89, or a pharmaceutically acceptable salt thereof, wherein
A is phenyl;
U is NH;
R¹⁰ is halo; and
R²⁰ is —(CH₂)₂O(CH₂)₂NHCH₃.

92. The compound of any one of claims 62 to 85, or a pharmaceutically acceptable salt thereof, wherein R¹-A′ is a proteolysis targeting chimera (PROTAC).

93. The compound of any one of claims 61 to 85 or 92, or a pharmaceutically acceptable salt thereof, wherein R¹ has the formula:
POI-L¹⁰⁰-CBN;
wherein:
POI is a compound that binds to a protein of interest;
L¹⁰⁰ is a PROTAC linker; and
CBN is a cereblon binding moiety.

94. The compound of claim 93, or a pharmaceutically acceptable salt thereof, wherein the protein of interest is a nuclear hormone receptor, a translation termination factor, a transcription factor, a cyclin-dependent kinase, a tyrosine kinase, a serine/threonine kinase, or an E3 ligase.

95. The compound of claim 93 or 94, or a pharmaceutically acceptable salt thereof, wherein the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

96. The conjugate of any one of claims 93 to 95, or a pharmaceutically acceptable salt thereof, wherein L¹⁰⁰ comprises one or more functional groups selected from glycol, alkyl, alkynyl, triazolyl, piperazinyl, piperidinyl, and combinations thereof.

97. The conjugate of any one of claims 93 to 96, or a pharmaceutically acceptable salt thereof, wherein CBN is selected from 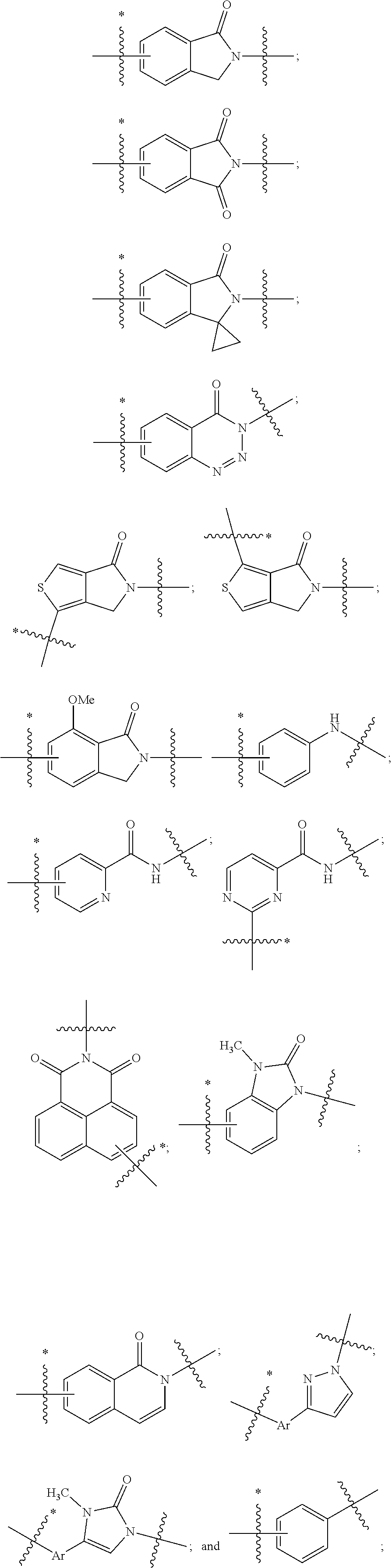
wherein [symbol]
indicates the point of attachment to A′; and [symbol]
indicates the point of attachment to L¹⁰⁰.

98. The compound of any one of claims 62 to 85 or 92 to 97, or a pharmaceutically acceptable salt thereof, wherein R¹ is selected from 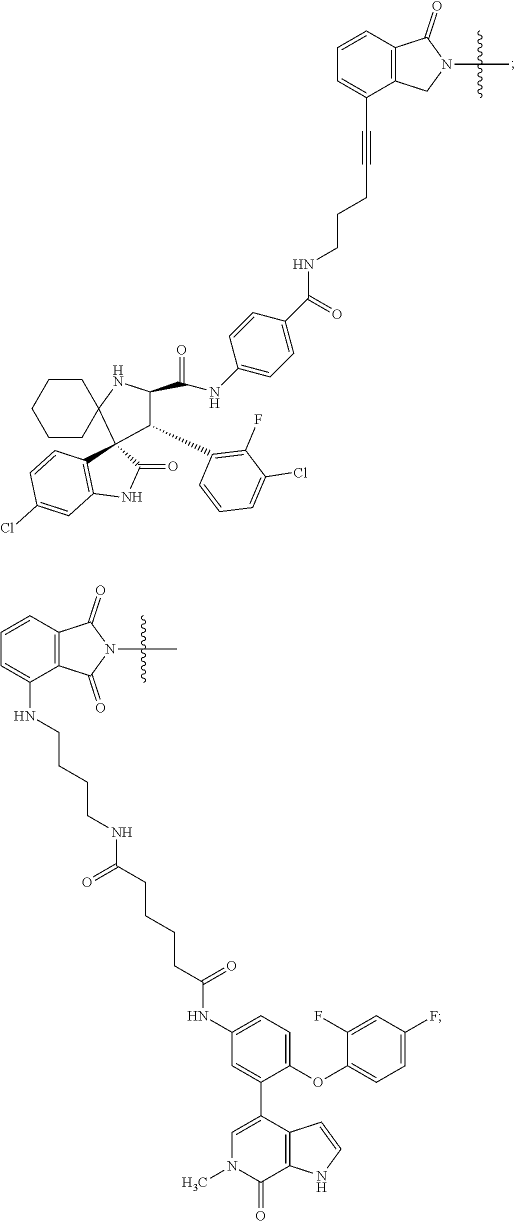 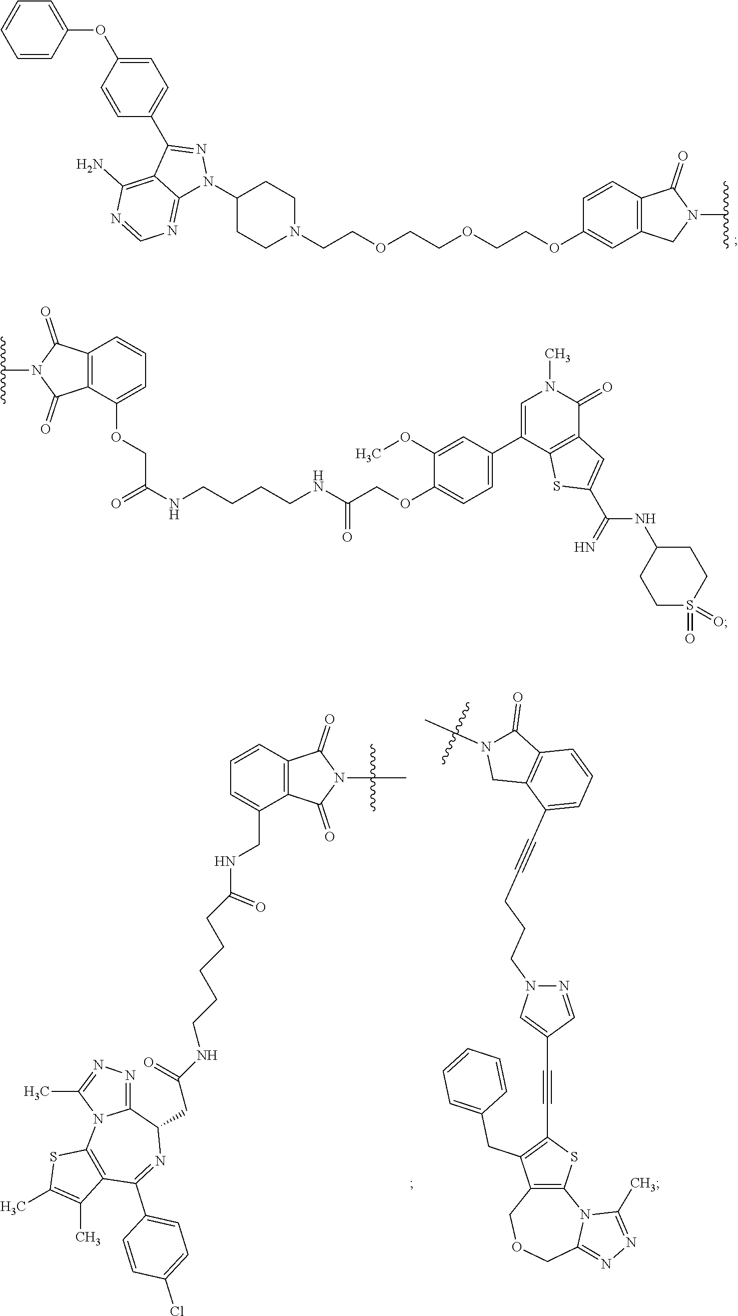 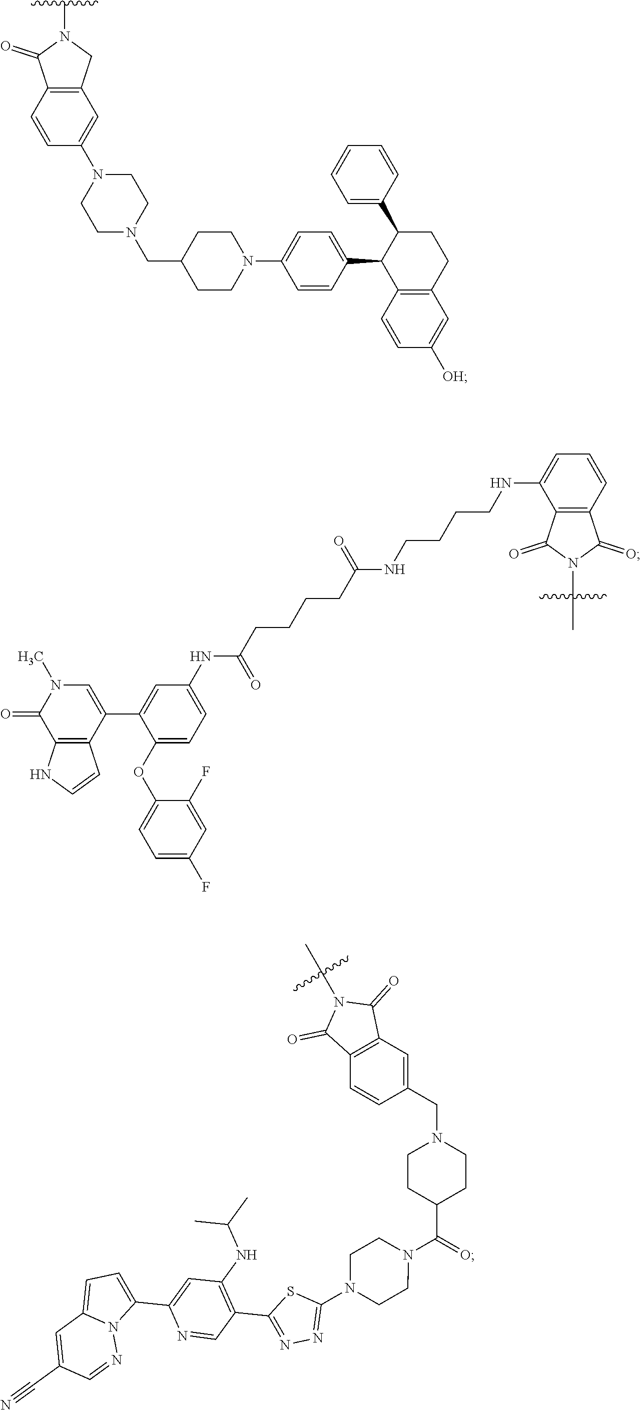 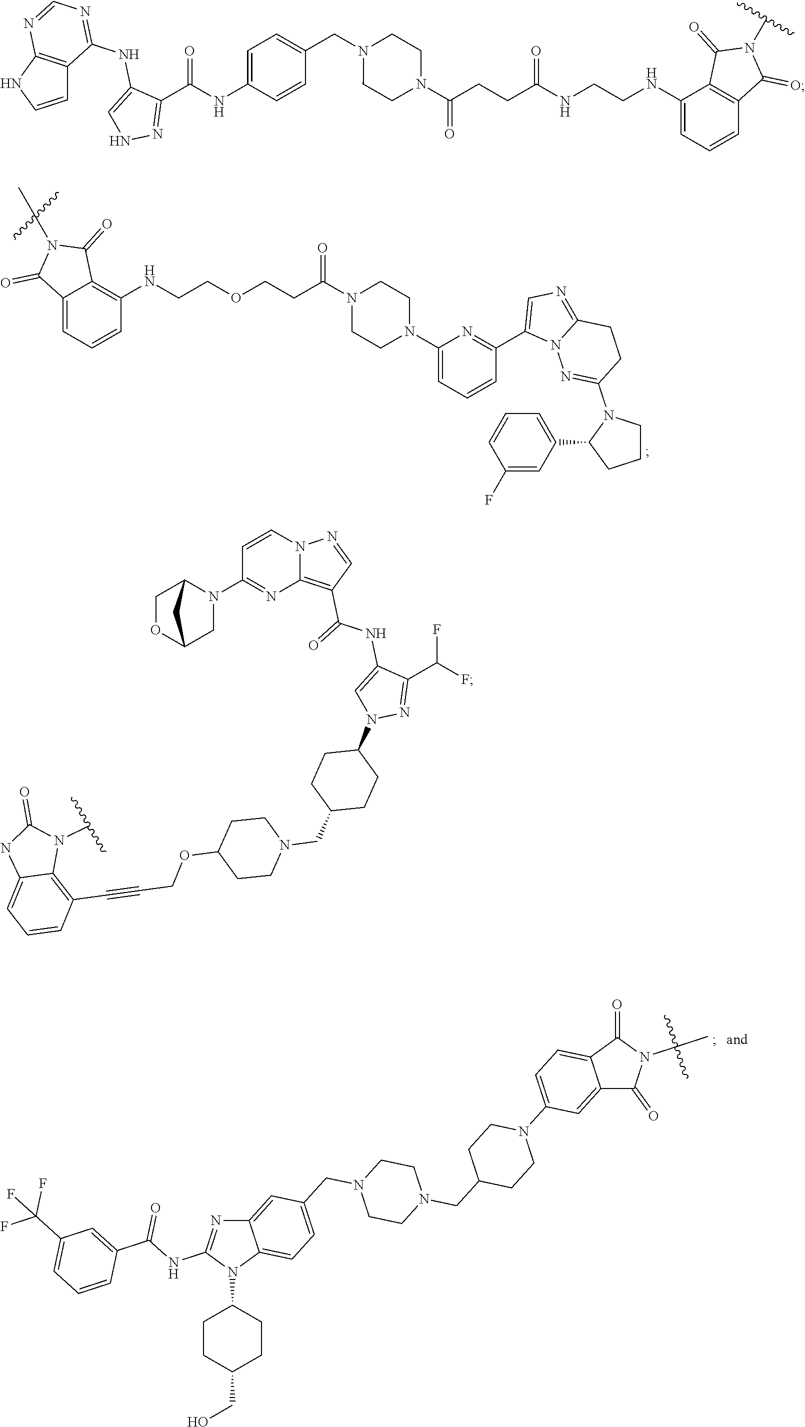 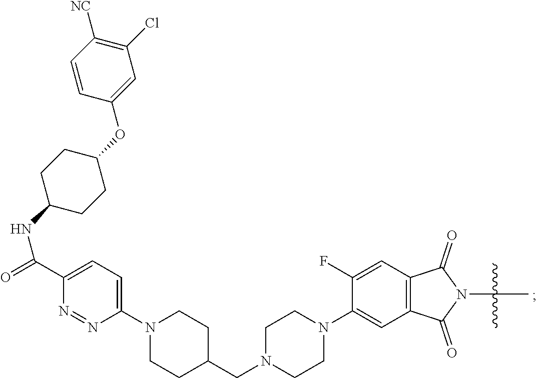
wherein [symbol] denotes the point of attachment to A′.

99. A method for preparing a conjugate of formula (XXXII): 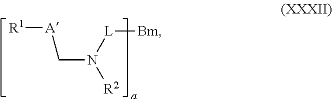
or a pharmaceutically acceptable salt thereof, wherein:
a is from 1 to 10;
A′ is 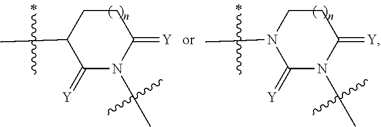
wherein
n is 0 or 1;
each Y is independently S or 0; [symbol]
indicates the point of attachment to R¹; and [symbol]
indicates the point of attachment to the methylene group;
R¹, together with A′, is a compound that induces a protein-protein interaction;
R² is selected from hydrogen, a group that provides stability to R¹-A′, a group that provides solubility to R¹-A′, and a group that provides stability and solubility to R¹-A′;
L is a cleavable linker; and
Bm is a binding moiety that is capable of specifically binding to a protein;
the method comprising:
reacting a compound of (XXXI) 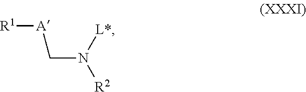
or a pharmaceutically acceptable salt thereof, wherein:
A′, R¹, and R² are as defined above and
L* is a cleavable linker precursor;
with a binding moiety that is capable of specifically binding to a protein.

100. The method of claim 99, further comprising attaching L* to a cysteine, lysine, tyrosine, or glutamine in the Bm.

101. The method of claim 100, wherein the cysteine or lysine is an engineered cysteine or lysine.

102. The method of claim 100, wherein the cysteine or lysine is endogenous to the Bm.

103. The method of claim 99, wherein the binding moiety is an antibody or an antigen binding portion thereof.

104. The method of claim 103, wherein L* is attached to an engineered cysteine at heavy chain position S239 and/or K334 of the antibody or antigen binding portion thereof according to EU numbering.

105. The method of claim 103, wherein L* is attached to the glutamine at heavy chain position 295 of the antibody or antigen binding portion thereof according to EU numbering.

106. The method of any one of claims 100 to 105, wherein the attaching is via site-specific conjugation.

107. The method of any one of claims 100 to 106, wherein (a) the protein that the Bm binds to is CD33 and R¹ binds to mouse double minute 2 homolog (MDM2), (b) the protein that the Bm binds to is prostate specific membrane antigen (PSMA) and R¹ binds to androgen receptor (AR), (c) the protein that the Bm binds to is CD33 and R¹ binds to bromodomain-containing protein 4 (BRD4), (d) the protein that the Bm binds to is HER2 and R¹ binds to G1 to S Phase Transition 1 (GSPT1), (e) the protein that the Bm binds to is CD33 and R¹ binds to GSPT1, (f) the protein that the Bm binds to is CD79b and R¹ binds to IRAK4, (g) the protein that the Bm binds to is HER2 and R¹ binds to BRD4, (h) the protein that the Bm binds to is BCMA and R¹ binds to BRD4, or (i) the protein that the Bm binds to is HER2 and R¹ binds to ER.

108. The method of any one of claims 99 to 107, wherein R¹-A′ is a targeted protein degrader.

109. The method of 108, wherein R¹ has the formula:
POI-L¹⁰⁰-CBN;
wherein:
POI is a compound that binds to a protein of interest;
L¹⁰⁰ is a PROTAC linker; and
CBN is a cereblon binding moiety.

110. The method of claim 109, wherein the protein of interest is a nuclear hormone receptor, a translation termination factor, a transcription factor, a cyclin-dependent kinase, a tyrosine kinase, a serine/threonine kinase, or an E3 ligase

111. The method of claim 109 or 110, wherein the protein of interest is selected from CD33, GSPT1, BRD4, AR, ER, IKZF1/3, CK1a, BCL-XL, IKZF2, IRAK4, BTK, STAT3, BTK and iMiD, BRD9, TRK, MDM2, CDK2/CDK9, CD97b, and EGFR.

112. The conjugate of any one of claims 109 to 111, or a pharmaceutically acceptable salt thereof, wherein L¹⁰⁰ comprises one or more functional groups selected from glycol, alkyl, alkynyl, triazolyl, piperazinyl, piperidinyl, and combinations thereof.

113. The method of any one of claims 109 to 112, or a pharmaceutically acceptable salt thereof, wherein CBN is selected from: 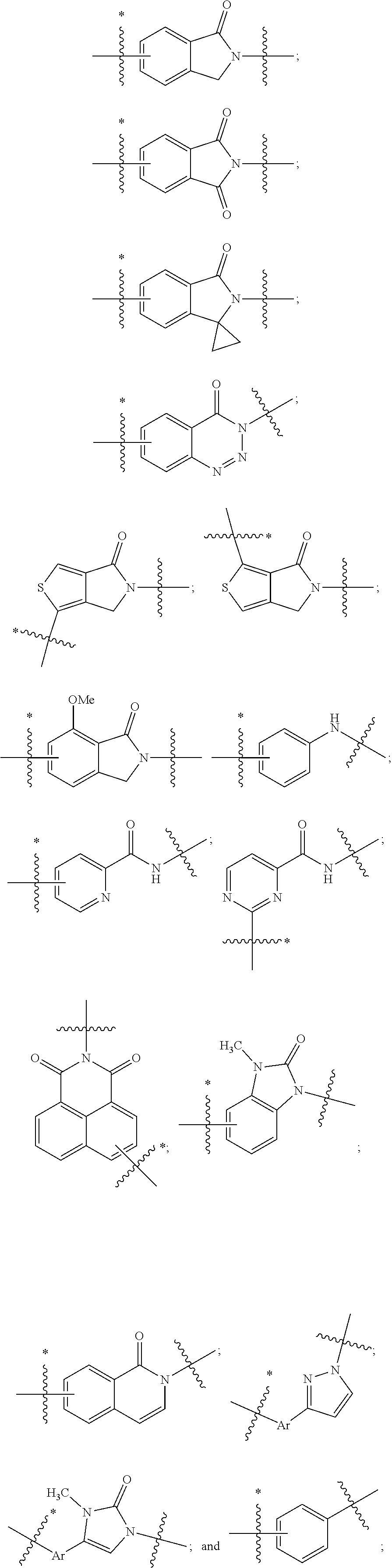
wherein [symbol]
indicates the point of attachment to A′; and [symbol]
indicates the point of attachment to L¹⁰⁰.

114. The method of any one of claims 109 to 113, wherein R¹ is selected from: 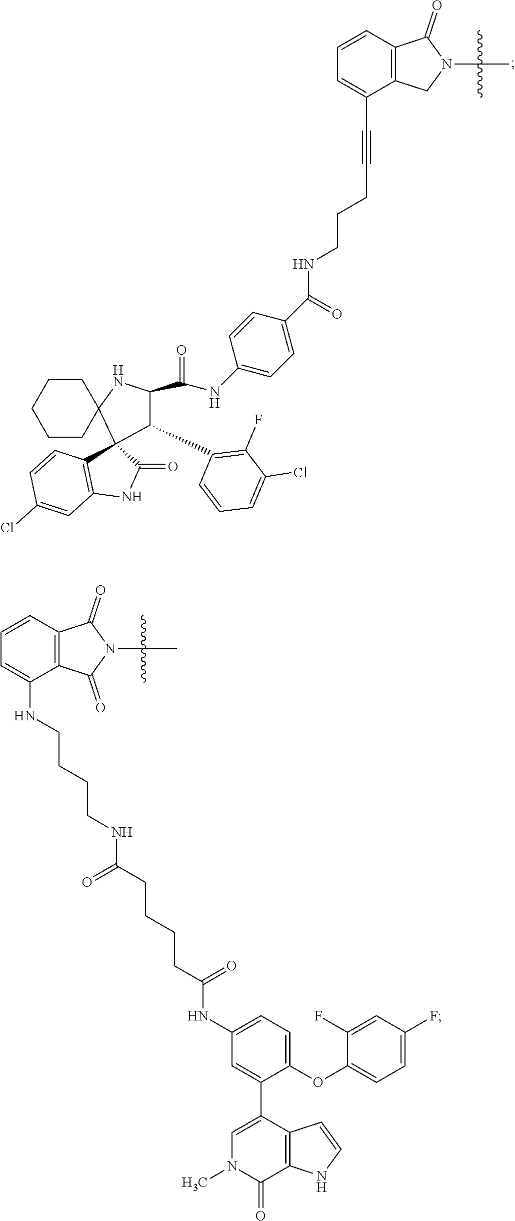 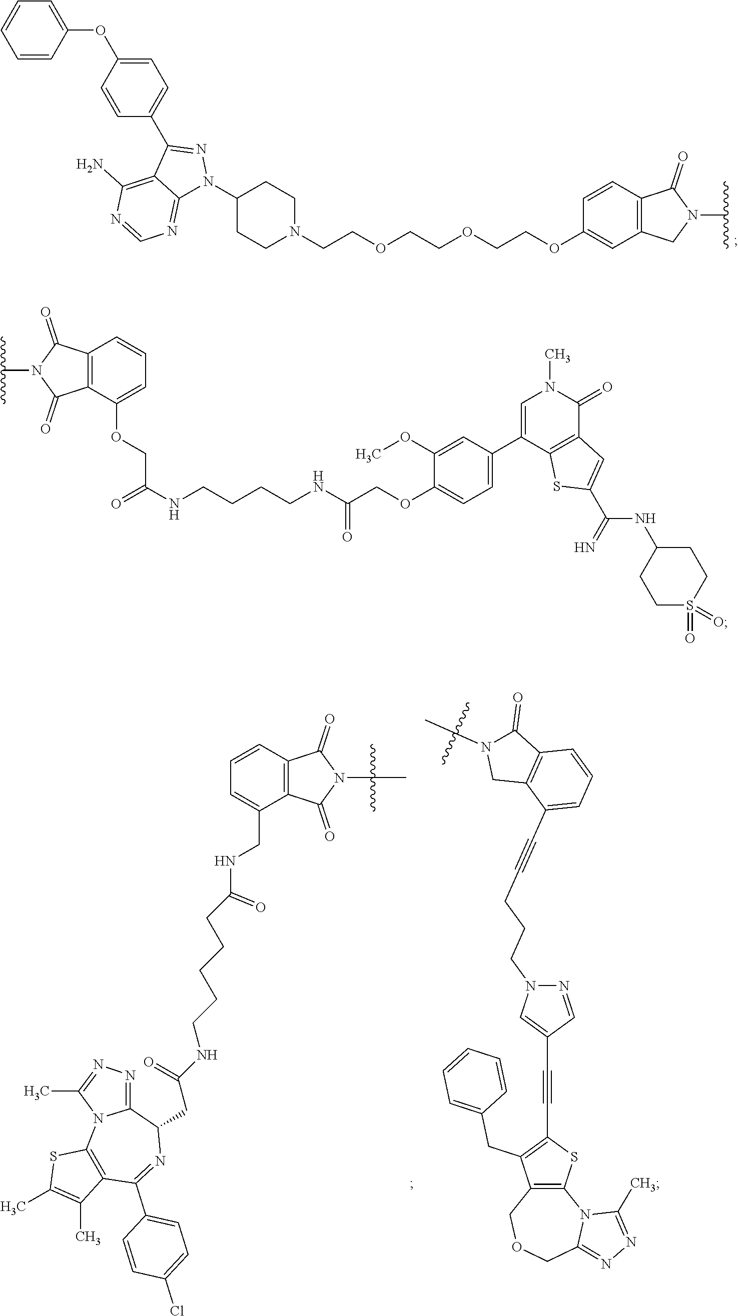 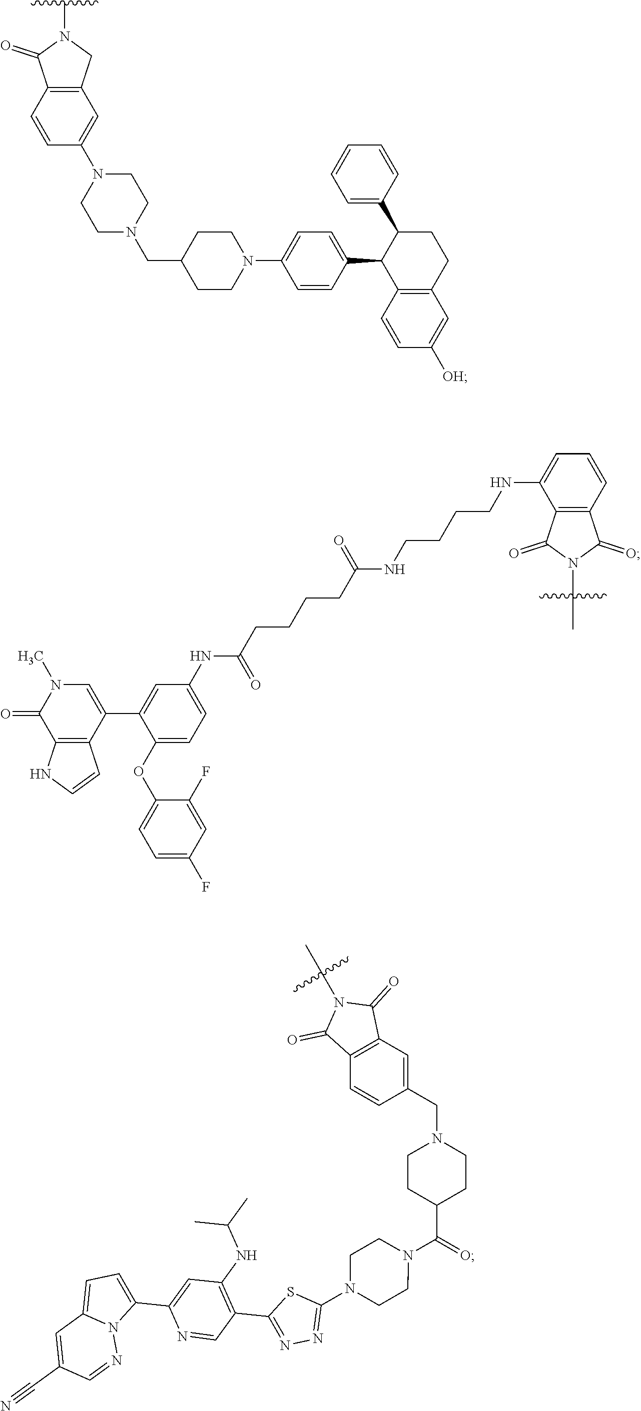 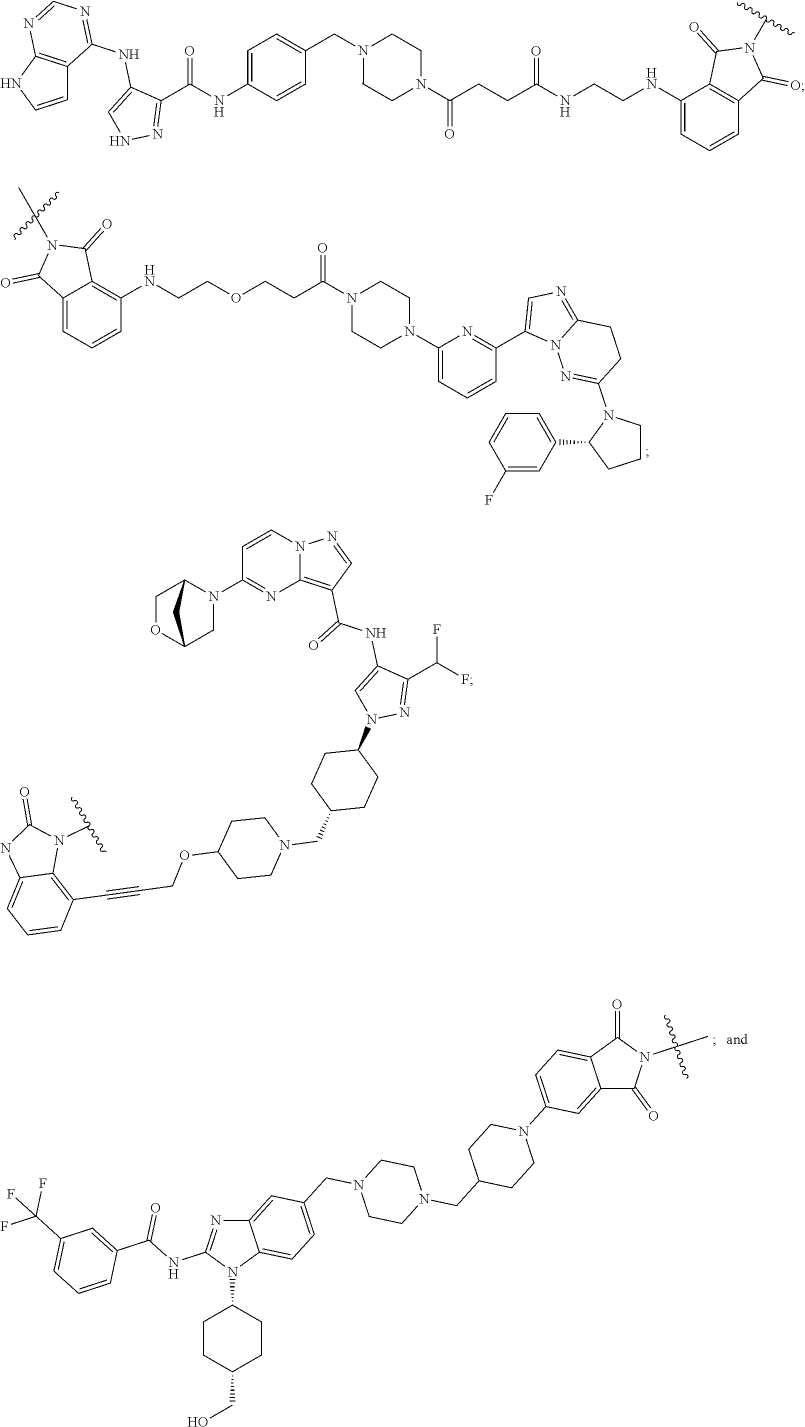 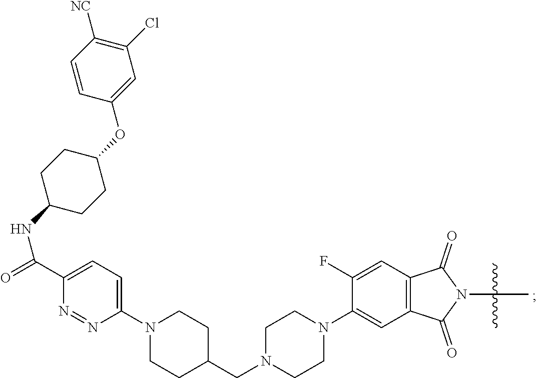
wherein [symbol] denotes the point of attachment to A′.

115. A conjugate made by the method of any one of claims 99 to 114.

116. A method of delivering a conjugate that induces a protein-protein interaction to a cell, the method comprising contacting the cell with a conjugate or composition of any one of claims 1 to 53 or 115, or a pharmaceutically acceptable salt thereof.

117. A method of delivering a conjugate of formula (XXXII): 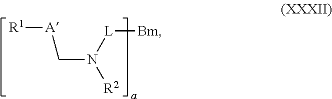
or a pharmaceutically acceptable salt thereof to a cell, wherein:
a is from 1 to 10;
A′ is 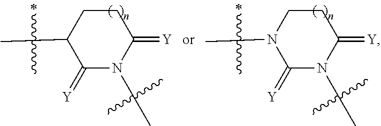
wherein
n is 0 or 1;
each Y is independently S or 0; [symbol]
indicates the point of attachment to R¹; and [symbol]
indicates the point of attachment to the methylene group;
R¹, together with A′, is a compound that induces a protein-protein interaction;
R² is selected from hydrogen, a group that provides stability to R¹-A′, a group that provides solubility to R¹-′A, and a group that provides stability and solubility to R¹-A′;
L is a cleavable linker; and
Bm is a binding moiety that is capable of specifically binding to a protein;
the method comprising contacting the cell with a conjugate of formula (XXXII), or a pharmaceutically acceptable salt thereof.
